SEQUENCE LISTING (394 sequences)

SEQ ID NO: 1 - protein, 422 aa, Homo sapiens
QRLPRMQEDSPLGGGSSGEDDPLGEEDLPSEEDSPREEDPPGEEDLPGEEDLPGEEDLPEVKPKSEEEGSLKLEDLPTVEAPGDPQEPQNNAHRDKEGDDQSHWRYGGDPPWPRVSPACAGRFQSPVDIRPQLAAFCPALRPLELLGFQLPPLPELRLRNNGHSVQLTLPPGLEMALGPGREYRALQLHLHWGAAGRPGSEHTVEGHRFPAEIHVVHLSTAFARVDEALGRPGGLAVLAAFLEEGPEENSAYEQLLSRLEEIAEEGSETQVPGLDISALLPSDFSRYFQYEGSLTTPPCAQGVIWTVFNQTVMLSAKQLHTLSDTLWGPGDSRLQLNFRATQPLNGRVIEASFPAGVDSSPRAAEPVQLNSCLAAGDILALVFGLLFAVTSVAFLVQMRRQHRRGTKGGVSYRPAEVAETGA

SEQ ID NO: 2 - protein, 459 aa, Homo sapiens
MAPLCPSPWLPLLIPAPAPGLTVQLLLSLLLLVPVHPQRLPRMQEDSPLGGGSSGEDDPLGEEDLPSEEDSPREEDPPGEEDLPGEEDLPGEEDLPEVKPKSEEEGSLKLEDLPTVEAPGDPQEPQNNAHRDKEGDDQSHWRYGGDPPWPRVSPACAGRFQSPVDIRPQLAAFCPALRPLELLGFQLPPLPELRLRNNGHSVQLTLPPGLEMALGPGREYRALQLHLHWGAAGRPGSEHTVEGHRFPAEIHVVHLSTAFARVDEALGRPGGLAVLAAFLEEGPEENSAYEQLLSRLEEIAEEGSETQVPGLDISALLPSDFSRYFQYEGSLTTPPCAQGVIWTVFNQTVMLSAKQLHTLSDTLWGPGDSRLQLNFRATQPLNGRVIEASFPAGVDSSPRAAEPVQLNSCLAAGDILALVFGLLFAVTSVAFLVQMRRQHRRGTKGGVSYRPAEVAETGA

SEQ ID NO: 3 - protein, 119 aa, synthetic construct
EVQLLESGGGLVQPGGSLKLSCAASGFTFSNYYMSWVRQAPGKGLEWVSAINSDGGITYYLDTVKGRFTISRDNSKNTLYLQMNSLRAEDTAVYYCARHRSGYFSMDYWGQGTTVTVSS

SEQ ID NO: 4 - protein, 119 aa, synthetic construct
LVQLVESGGGLVKPGGSLRLSCAASGFTFSNYYMSWIRQAPGKGLEWVSAINSDGGITYYLDTVKGRFTISRDNAKNSLYLQMNSLRAEDTAVYYCARHRSGYFSMDYWGQGTLVTVSS

SEQ ID NO: 5 - protein, 119 aa, synthetic construct
EVQLVESGGGLVQPGGSLRLSCAASGFTFSNYYMSWVRQAPGKGLEWVAAINSDGGITYYLDTVKGRFTISRDNAKNSLYLQMNSLRAEDTAVYYCARHRSGYFSMDYWGQGTLVTVSS

SEQ ID NO: 6 - protein, 119 aa, synthetic construct
EVQLLESGGGLVQPGGSLRLSCAASGFTFSNYYMSWVRQAPGKGLEWVSAINSDGGITYYLDTVKGRFTISRDDSKNTLYLQMSSLRAEDTAVYYCARHRSGYFSMDYWGQGTTVTVSS

SEQ ID NO: 7 - protein, 119 aa, synthetic construct
EVRLVESGGGLVKPGGSLRLSCAASGFTFSNYYMSWIRQAPEKRLELVSAINSDGGITYYLDTVKGRFTISRDNAKNSLYLQMNSLRAEDTALFYCARHRSGYFSMDYWGQGTSVTVSS

SEQ ID NO: 8 - protein, 119 aa, synthetic construct
EVRLVESGGGLVKPGGSLRLSCAASGFTFSNYYMSWIRQAPEKRLELVSAINSDGAITYYLDTVKGRFTISRDNAKNSLYLQMNSLRAEDTALFYCARHRSGYFSMDYWGQGTSVTVSS

SEQ ID NO: 9 - protein, 119 aa, synthetic construct
EVRLVESGGGLVKPGGSLRLSCAVSGFTFSNYYMSWIRQAPEKRLELVSAINSDGGITYYLDTVKGRFTISRDNAKNSLYLQMNSLRAEDTALFYCARHRSGYFSMDYWGQGTSVTVSS

SEQ ID NO: 10 - protein, 107 aa, synthetic construct
DIVMTQSPATLSLSPGDRATLSCKASQNVVSAVAWYQQKPGQAPRLLIYSASNRYTGIPARFSGSGSGTDFTLTISSLQSEDFAVYYCQQYSNYPWTFGGGTKVEIK

SEQ ID NO: 11 - protein, 107 aa, synthetic construct
EIVMTQSPATLSVSPGERATLSCKASQNVVSAVAWYQQKPGQAPRLLVYSASNRYTGIPDRFSGSGSGTEFTLTISSLQSEDFAVYYCQQYSNYPWTFGQGTKLEIK

SEQ ID NO: 12 - protein, 107 aa, synthetic construct
EIVMTQSPATLSVSPGERATLSCKASQNVVSAVAWYQQKPGQSPRLLIYSASNRYTGIPARFSGSGSGTEFTLTISSLQSEDFAAYYCQQYSNYPWTFGGGTKVEIK

SEQ ID NO: 13 - protein, 107 aa, synthetic construct
DIQMTQSPFSLSASVGDRVTITCKASQNVVSAVAWYQQKPGKAPKLLIYSASNRYTGVPSRFSGSGSGTDFTLTISSLQPEDFATYFCQQYSNYPWTFGGGTKLEIK

SEQ ID NO: 14 - protein, 107 aa, synthetic construct
AIQLTQSQRFLSASVGDRVTITCRASQNVVSALAWYQQKPGQSPKLLIYSASNRYTGVPSRFSGSGSGTDFTLTISSLQPEDFADFFCQQYSNYPWTFGGGTKLEIK

SEQ ID NO: 15 - protein, 107 aa, synthetic construct
EIVMTQSPATLSVSPGERATLSCKASQNVVSAVAWYQQKPGQSPRLLIYSASNRYTGIPARFSGSGSGTEFTLTISSLQSEDFAAYYCQQYRNYPWTFGGGTKVEIK

SEQ ID NO: 16 - protein, 107 aa, synthetic construct
EIVMTQSPATLSVSPGERATLSCKASQNVVSAVGWYQQKPGQSPRLLIYSASNRYTGIPARFSGSGSGTEFTLTISSLQSEDFAAYYCQQYSNYPWTFGGGTKVEIK

SEQ ID NO: 17 - protein, 107 aa, synthetic construct
EIVMTQSPATLSVSPGERVTLSCKASQNVVSAVGWYQKKPGQSPRLLIYSASNRYTGIPARFSGSGSGTEFTLTISSLQSEDFAAYYCQQYNNYPWTFGGGTKVEIK

SEQ ID NO: 18 - DNA, 357 bp, synthetic construct
  source 1..357: mol_type = other DNA
gaggtgcagctgctggaatctggaggaggccttgtgcaacctggaggcagcctgaagctgagctgtgctgcctctggcttcaccttcagcaactactacatgagctgggtgagacaagcccctggcaagggcctggagtgggtgtctgccatcaactctgatggaggcatcacctactacctggacacagtgaagggcagattcaccatcagcagagataatagcaaaaacaccctgtacctgcagatgaacagcctgagagctgaggacacagctgtgtactactgtgctagacacagatctggctacttcagcatggactactggggccaaggcaccacagtgactgtgagctct

SEQ ID NO: 19 - DNA, 357 bp, synthetic construct
  source 1..357: mol_type = other DNA
ctggtgcaactggtagaatctggaggaggccttgtgaaacctggaggcagcctgagactgagctgtgctgcctctggcttcaccttcagcaactactacatgagctggatcagacaagcccctggcaagggcctggagtgggtgtctgccatcaactctgatggaggcatcacctactacctggacacagtgaagggcagattcaccatcagcagagataatgccaaaaacagcctgtacctgcagatgaacagcctgagagctgaggacacagctgtgtactactgtgctagacacagatctggctacttcagcatggactactggggccaaggcaccctggtgactgtgagctct

SEQ ID NO: 20 - DNA, 357 bp, synthetic construct
  source 1..357: mol_type = other DNA
gaggtgcaactggtagaatctggaggaggccttgtgcagcctggaggcagcctgagactgagctgtgctgcctctggcttcaccttcagcaactactacatgagctgggtgagacaagcccctggcaagggcctggagtgggtggctgccatcaactctgatggaggcatcacctactacctggacacagtgaagggcagattcaccatcagcagagataatgccaaaaacagcctgtacctgcagatgaacagcctgagagctgaggacacagctgtgtactactgtgctagacacagatctggctacttcagcatggactactggggccaaggcaccctggtgactgtgagctct

SEQ ID NO: 21 - DNA, 357 bp, synthetic construct
  source 1..357: mol_type = other DNA
gaggtgcagctgctggaatctggaggaggccttgtgcaacctggaggcagcctgagactgagctgtgctgcctctggcttcaccttcagcaactactacatgagctgggtgagacaagcccctggcaagggcctggagtgggtgtctgccatcaactctgatggaggcatcacctactacctggacacagtgaagggcagattcaccatcagcagagatgacagcaaaaacaccctgtacctgcagatgagcagcctgagagctgaggacacagctgtgtactactgtgctagacacagatctggctacttcagcatggactactggggccaaggcaccacagtgactgtgagctct

SEQ ID NO: 22 - DNA, 357 bp, synthetic construct
  source 1..357: mol_type = other DNA
gaggtcagactggtagaatctggaggaggccttgtgaaacctggaggcagcctgagactgagctgtgctgcctctggcttcaccttcagcaactactacatgagctggatcagacaagcccctgagaagagactggagctggtgtctgccatcaactctgatggaggcatcacctactacctggacacagtgaagggcagattcaccatcagcagagataatgccaaaaacagcctgtacctgcagatgaacagcctgagagctgaggacacagccctgttctactgtgctagacacagatctggctacttcagcatggactactggggccaaggcacctctgtgactgtgagctct

SEQ ID NO: 23 - DNA, 357 bp, synthetic construct
  source 1..357: mol_type = other DNA
gaagtccgtctggtcgagagcggggggggcctggtgaagcctggcggcagcctgaggctgtcttgcgccgcttccggcttcaccttttctaactactatatgtcctggatcagacaggccccagagaagaggctggagctggtgtccgccatcaactctgacggcggcatcacatactatctggataccgtgaagggcagattcacaatctctcgcgataacgccaagaactccctgtacctccagatgaacagcctgcgggccgaggacaccgctctgttctattgcgctaggcaccggtccggctactttagcatggattattggggccagggcacctccgtcacagtgtccagc

SEQ ID NO: 24 - DNA, 321 bp, synthetic construct
  source 1..321: mol_type = other DNA
gacattgtgatgacacagtctcctgccaccctgagcctgagccctggagacagagctaccctgagctgcaaggcttctcagaatgtggtgtctgctgtggcctggtatcagcagaagcctggacaagcccctagactgctgatctactctgctagcaacagatacactggaatccctgctagattctctggctctggctctggcacagacttcaccctgaccatcagcagcctgcagtctgaggactttgctgtgtactactgtcagcagtacagcaactacccttggacctttggaggaggcaccaaggtggagatcaag

SEQ ID NO: 25 - DNA, 321 bp, synthetic construct
  source 1..321: mol_type = other DNA
gagattgtgatgacacagtctcctgccaccctgtctgtgagccctggagagagagctaccctgagctgcaaggcttctcagaatgtggtgtctgctgtggcctggtatcagcagaagcctggacaagcccctagactgctggtgtactctgctagcaacagatacactggaatccctgacagattctctggctctggctctggcacagagttcaccctgaccatcagcagcctgcagtctgaggactttgctgtgtactactgtcagcagtacagcaactacccttggacctttggacaaggcaccaagctggagatcaag

SEQ ID NO: 26 - DNA, 321 bp, synthetic construct
  source 1..321: mol_type = other DNA
gagattgtgatgacacagtctcctgccaccctgtctgtgagccctggagagagagctaccctgagctgcaaggcttctcagaatgtggtgtctgctgtggcctggtatcagcagaagcctggacagagccctagactgctgatctactctgctagcaacagatacactggaatccctgctagattctctggctctggctctggcacagagttcaccctgaccatcagcagcctgcagtctgaggactttgctgcctactactgtcagcagtacagcaactacccttggacctttggaggaggcaccaaggtggagatcaag

SEQ ID NO: 27 - DNA, 321 bp, synthetic construct
  source 1..321: mol_type = other DNA
gaaattgtgatgactcagagccccgcaactctgagcgtgtctcccggcgagagagccaccctgtcttgcaaggcttcccagaacgtggtgtctgccgtggcttggtatcagcagaagccaggccagtccccaaggctgctgatctactccgccagcaataggtataccggcatccctgctcggttctctggctccggcagcggcacagagtttaccctgaccatctcctccctccagagcgaggacttcgccgcttactattgccagcagtactctaactatccttggacctttggcggcggcacaaaggtggagatcaag

SEQ ID NO: 28 - DNA, 321 bp, synthetic construct
  source 1..321: mol_type = other DNA
gacattcagatgacacagtctcctttcagcctgtctgcctctgtgggagacagagtgaccatcacctgcaaggcttctcagaatgtggtgtctgctgtggcctggtatcagcagaagcctggaaaggcccctaagctgctgatctactctgctagcaacagatacactggagtgcctagcagattctctggctctggctctggcacagacttcaccctgaccatcagcagcctgcagcctgaggactttgccacctacttctgtcagcagtacagcaactacccttggacctttggaggaggcaccaagctggagatcaag

SEQ ID NO: 29 - DNA, 321 bp, synthetic construct
  source 1..321: mol_type = other DNA
gccattcagctgacacagtctcagagattcctgtctgcctctgtgggagacagagtgaccatcacctgcagagcttctcagaatgtggtgtctgctctggcctggtatcagcagaagcctggacagagccctaagctgctgatctactctgctagcaacagatacactggagtgcctagcagattctctggctctggctctggcacagacttcaccctgaccatcagcagcctgcagcctgaggactttgctgacttcttctgtcagcagtacagcaactacccttggacctttggaggaggcaccaagctggagatcaag

SEQ ID NO: 30 - protein, 219 aa, Homo sapiens
MCPARSLLLVATLVLLDHLSLARNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 31 - protein, 197 aa, Homo sapiens
RNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 32 - protein, 328 aa, Homo sapiens
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 33 - protein, 306 aa, Homo sapiens
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 34 - protein, 662 aa, Homo sapiens
MEPLVTWVVPLLFLFLLSRQGAACRTSECCFQDPPYPDADSGSASGPRDLRCYRISSDRYECSWQYEGPTAGVSHFLRCCLSSGRCCYFAAGSATRLQFSDQAGVSVLYTVTLWVESWARNQTEKSPEVTLQLYNSVKYEPPLGDIKVSKLAGQLRMEWETPDNQVGAEVQFRHRTPSSPWKLGDCGPQDDDTESCLCPLEMNVAQEFQLRRRQLGSQGSSWSKWSSPVCVPPENPPQPQVRFSVEQLGQDGRRRLTLKEQPTQLELPEGCQGLAPGTEVTYRLQLHMLSCPCKAKATRTLHLGKMPYLSGAAYNVAVISSNQFGPGLNQTWHIPADTHTEPVALNISVGTNGTTMYWPARAQSMTYCIEWQPVGQDGGLATCSLTAPQDPDPAGMATYSWSRESGAMGQEKCYYITIFASAHPEKLTLWSTVLSTYHFGGNASAAGTPHHVSVKNHSLDSVSVDWAPSLLSTCPGVLKEYVVRCRDEDSKQVSEHPVQPTETQVTLSGLRAGVAYTVQVRADTAWLRGVWSQPQRFSIEVQVSDWLIFFASLGSFLSILLVGVLGYLGLNRAARHLCPPLPTPCASSAIEFPGGKETWQWINPVDFQEEASLQEALVVEMSWDKGERTEPLEKTELPEGAPELALDTELSLEDGDRCKAKM

SEQ ID NO: 35 - protein, 639 aa, Homo sapiens
CRTSECCFQDPPYPDADSGSASGPRDLRCYRISSDRYECSWQYEGPTAGVSHFLRCCLSSGRCCYFAAGSATRLQFSDQAGVSVLYTVTLWVESWARNQTEKSPEVTLQLYNSVKYEPPLGDIKVSKLAGQLRMEWETPDNQVGAEVQFRHRTPSSPWKLGDCGPQDDDTESCLCPLEMNVAQEFQLRRRQLGSQGSSWSKWSSPVCVPPENPPQPQVRFSVEQLGQDGRRRLTLKEQPTQLELPEGCQGLAPGTEVTYRLQLHMLSCPCKAKATRTLHLGKMPYLSGAAYNVAVISSNQFGPGLNQTWHIPADTHTEPVALNISVGTNGTTMYWPARAQSMTYCIEWQPVGQDGGLATCSLTAPQDPDPAGMATYSWSRESGAMGQEKCYYITIFASAHPEKLTLWSTVLSTYHFGGNASAAGTPHHVSVKNHSLDSVSVDWAPSLLSTCPGVLKEYVVRCRDEDSKQVSEHPVQPTETQVTLSGLRAGVAYTVQVRADTAWLRGVWSQPQRFSIEVQVSDWLIFFASLGSFLSILLVGVLGYLGLNRAARHLCPPLPTPCASSAIEFPGGKETWQWINPVDFQEEASLQEALVVEMSWDKGERTEPLEKTELPEGAPELALDTELSLEDGDRCKAKM

SEQ ID NO: 36 - protein, 862 aa, Homo sapiens
MAHTFRGCSLAFMFIITWLLIKAKIDACKRGDVTVKPSHVILLGSTVNITCSLKPRQGCFHYSRRNKLILYKFDRRINFHHGHSLNSQVTGLPLGTTLFVCKLACINSDEIQICGAEIFVGVAPEQPQNLSCIQKGEQGTVACTWERGRDTHLYTEYTLQLSGPKNLTWQKQCKDIYCDYLDFGINLTPESPESNFTAKVTAVNSLGSSSSLPSTFTFLDIVRPLPPWDIRIKFQKASVSRCTLYWRDEGLVLLNRLRYRPSNSRLWNMVNVTKAKGRHDLLDLKPFTEYEFQISSKLHLYKGSWSDWSESLRAQTPEEEPTGMLDVWYMKRHIDYSRQQISLFWKNLSVSEARGKILHYQVTLQELTGGKAMTQNITGHTSWTTVIPRTGNWAVAVSAANSKGSSLPTRINIMNLCEAGLLAPRQVSANSEGMDNILVTWQPPRKDPSAVQEYVVEWRELHPGGDTQVPLNWLRSRPYNVSALISENIKSYICYEIRVYALSGDQGGCSSILGNSKHKAPLSGPHINAITEEKGSILISWNSIPVQEQMGCLLHYRIYWKERDSNSQPQLCEIPYRVSQNSHPINSLQPRVTYVLWMTALTAAGESSHGNEREFCLQGKANWMAFVAPSICIAIIMVGIFSTHYFQQKVFVLLAALRPQWCSREIPDPANSTCAKKYPIAEEKTQLPLDRLLIDWPTPEDPEPLVISEVLHQVTPVFRHPPCSNWPQREKGIQGHQASEKDMMHSASSPPPPRALQAESRQLVDLYKVLESRGSDPKPENPACPWTVLPAGDLPTHDGYLPSNIDDLPSHEAPLADSLEELEPQHISLSVFPSSSLHPLTFSCGDKLTLDQLKMRCDSLML

SEQ ID NO: 37 - protein, 839 aa, Homo sapiens
KIDACKRGDVTVKPSHVILLGSTVNITCSLKPRQGCFHYSRRNKLILYKFDRRINFHHGHSLNSQVTGLPLGTTLFVCKLACINSDEIQICGAEIFVGVAPEQPQNLSCIQKGEQGTVACTWERGRDTHLYTEYTLQLSGPKNLTWQKQCKDIYCDYLDFGINLTPESPESNFTAKVTAVNSLGSSSSLPSTFTFLDIVRPLPPWDIRIKFQKASVSRCTLYWRDEGLVLLNRLRYRPSNSRLWNMVNVTKAKGRHDLLDLKPFTEYEFQISSKLHLYKGSWSDWSESLRAQTPEEEPTGMLDVWYMKRHIDYSRQQISLFWKNLSVSEARGKILHYQVTLQELTGGKAMTQNITGHTSWTTVIPRTGNWAVAVSAANSKGSSLPTRINIMNLCEAGLLAPRQVSANSEGMDNILVTWQPPRKDPSAVQEYVVEWRELHPGGDTQVPLNWLRSRPYNVSALISENIKSYICYEIRVYALSGDQGGCSSILGNSKHKAPLSGPHINAITEEKGSILISWNSIPVQEQMGCLLHYRIYWKERDSNSQPQLCEIPYRVSQNSHPINSLQPRVTYVLWMTALTAAGESSHGNEREFCLQGKANWMAFVAPSICIAIIMVGIFSTHYFQQKVFVLLAALRPQWCSREIPDPANSTCAKKYPIAEEKTQLPLDRLLIDWPTPEDPEPLVISEVLHQVTPVFRHPPCSNWPQREKGIQGHQASEKDMMHSASSPPPPRALQAESRQLVDLYKVLESRGSDPKPENPACPWTVLPAGDLPTHDGYLPSNIDDLPSHEAPLADSLEELEPQHISLSVFPSSSLHPLTFSCGDKLTLDQLKMRCDSLML

SEQ ID NO: 38 - protein, 306 aa, synthetic construct
IWELKKDVYVVELDAYPDAPGEMVVLTCDTPEEDGITWTLDQSSEVLGSGKTLTIQVKEAGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLAYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 39 - protein, 306 aa, synthetic construct
IWELKKDVYVVELDAYPDAPGEMVVLTCDTPEEDGITWTLDQSSEVLGSGKTLTIQVKEAGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHALKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 40 - protein, 306 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHAKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHALAYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 41 - protein, 306 aa, synthetic construct
IWELKKDVYVVELDAYPDAPGEMVVLTCDTPEEDGITWTLDQSSEVLGSGKTLTIQVKEAGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 42 - protein, 306 aa, synthetic construct
IWELKKDVYVVELDAYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHALKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 43 - protein, 306 aa, synthetic construct
IWELKKDVYVVELDAYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLAYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 44 - protein, 306 aa, synthetic construct
IWELKKDVYVVELDAYPDAPGEMVVLTCDTPEEDGITWTLDQSSEVLGSGKTLTIQVKEFGDAGQYTCHKGGEVLSHSLLLLHAKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 45 - protein, 306 aa, synthetic construct
IWELKKDVYVVELDWYPDAPGEMVVLTCDTPEEDGITWTLDQSSEVLGSGKTLTIQVKEAGDAGQYTCHKGGEVLSHSLLLLHAKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 46 - protein, 306 aa, synthetic construct
IWELKKDVYVVELDWYPDAPGEMVVLTCDTPEEDGITWTLDQSSEVLGSGKTLTIQVKEAGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHALKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 47 - protein, 306 aa, synthetic construct
IWELKKDVYVVELDWYPDAPGEMVVLTCDTPEEDGITWTLDQSSEVLGSGKTLTIQVKEAGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLAYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 48 - protein, 306 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHALAYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 49 - protein, 306 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHAKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHALKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 50 - protein, 306 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHAKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLAYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 51 - protein, 305 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHIKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 52 - protein, 328 aa, synthetic construct
MCHQQLVISWFSLVFLASPLVAIWELKKDVYVVELDAYPDAPGEMVVLTCDTPEEDGITWTLDQSSEVLGSGKTLTIQVKEAGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLAYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 53 - protein, 328 aa, synthetic construct
MCHQQLVISWFSLVFLASPLVAIWELKKDVYVVELDAYPDAPGEMVVLTCDTPEEDGITWTLDQSSEVLGSGKTLTIQVKEAGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHALKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 54 - protein, 328 aa, synthetic construct
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHAKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHALAYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 55 - protein, 328 aa, synthetic construct
MCHQQLVISWFSLVFLASPLVAIWELKKDVYVVELDAYPDAPGEMVVLTCDTPEEDGITWTLDQSSEVLGSGKTLTIQVKEAGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 56 - protein, 328 aa, synthetic construct
MCHQQLVISWFSLVFLASPLVAIWELKKDVYVVELDAYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHALKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 57 - protein, 328 aa, synthetic construct
MCHQQLVISWFSLVFLASPLVAIWELKKDVYVVELDAYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLAYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 58 - protein, 328 aa, synthetic construct
MCHQQLVISWFSLVFLASPLVAIWELKKDVYVVELDAYPDAPGEMVVLTCDTPEEDGITWTLDQSSEVLGSGKTLTIQVKEFGDAGQYTCHKGGEVLSHSLLLLHAKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 59 - protein, 328 aa, synthetic construct
MCHQQLVISWFSLVFLASPLVAIWELKKDVYVVELDWYPDAPGEMVVLTCDTPEEDGITWTLDQSSEVLGSGKTLTIQVKEAGDAGQYTCHKGGEVLSHSLLLLHAKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 60 - protein, 328 aa, synthetic construct
MCHQQLVISWFSLVFLASPLVAIWELKKDVYVVELDWYPDAPGEMVVLTCDTPEEDGITWTLDQSSEVLGSGKTLTIQVKEAGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHALKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 61 - protein, 328 aa, synthetic construct
MCHQQLVISWFSLVFLASPLVAIWELKKDVYVVELDWYPDAPGEMVVLTCDTPEEDGITWTLDQSSEVLGSGKTLTIQVKEAGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLAYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 62 - protein, 328 aa, synthetic construct
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHALAYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 63 - protein, 328 aa, synthetic construct
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHAKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHALKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 64 - protein, 328 aa, synthetic construct
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHAKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLAYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 65 - protein, 327 aa, synthetic construct
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHIKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 66 - protein, 4 aa, synthetic construct
GGGS

SEQ ID NO: 67 - protein, 8 aa, synthetic construct
GGGSGGGS

SEQ ID NO: 68 - protein, 12 aa, synthetic construct
GGGSGGGSGGGS

SEQ ID NO: 69 - protein, 16 aa, synthetic construct
GGGSGGGSGGGSGGGS

SEQ ID NO: 70 - protein, 5 aa, synthetic construct
GGGGS

SEQ ID NO: 71 - protein, 10 aa, synthetic construct
GGGGSGGGGS

SEQ ID NO: 72 - protein, 15 aa, synthetic construct
GGGGSGGGGSGGGGS

SEQ ID NO: 73 - protein, 20 aa, synthetic construct
GGGGSGGGGSGGGGSGGGGS

SEQ ID NO: 74 - protein, 8 aa, synthetic construct
GGGGGGGS

SEQ ID NO: 75 - protein, 16 aa, synthetic construct
GGGGGGGSGGGGGGGS

SEQ ID NO: 76 - protein, 24 aa, synthetic construct
GGGGGGGSGGGGGGGSGGGGGGGS

SEQ ID NO: 77 - protein, 32 aa, synthetic construct
GGGGGGGSGGGGGGGSGGGGGGGSGGGGGGGS

SEQ ID NO: 78 - protein, 5 aa, synthetic construct
SGGGG

SEQ ID NO: 79 - protein, 10 aa, synthetic construct
SGGGGSGGGG

SEQ ID NO: 80 - protein, 15 aa, synthetic construct
SGGGGSGGGGSGGGG

SEQ ID NO: 81 - protein, 20 aa, synthetic construct
SGGGGSGGGGSGGGGSGGGG

SEQ ID NO: 82 - protein, 22 aa, Homo sapiens
MCHQQLVISWFSLVFLASPLVA

SEQ ID NO: 83 - protein, 20 aa, Homo sapiens
MYRMQLLSCIALSLALVTNS

SEQ ID NO: 84 - protein, 25 aa, Homo sapiens
MGVLLTQRTLLSLVLALLFPSMASM

SEQ ID NO: 85 - protein, 18 aa, Homo sapiens
MASLWLLSCFSLVGAAFG

SEQ ID NO: 86 - protein, 15 aa, Homo sapiens
MNLLLILTFVAAAVA

SEQ ID NO: 87 - protein, 24 aa, Homo sapiens
MALWMRLLPLLALLALWGPDPAAA

SEQ ID NO: 88 - protein, 12 aa, Homo sapiens
VQVQGKSKREKK

SEQ ID NO: 89 - skipped

SEQ ID NO: 90 - protein, 306 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGASAAEAADAVFTDKTSATVICRKNASISVRAQDRYYSSSWSEWASVPCS

SEQ ID NO: 91 - protein, 201 aa, synthetic construct
FLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 92 - protein, 185 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEEIES

SEQ ID NO: 93 - protein, 306 aa, synthetic construct
IWELKKDVYVVELDWYPDAPGEMVVLTCDTPEEDGITWTLDQSSEVLGSGKTLTIQVKKFGDAGQYTCHKGGEVLSHSLLLLHKKEDGIWSTDILKDQEEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKEDRVFTDKTSATVICRKNASISVRAQDRYYSSSWSEWASVPCS

SEQ ID NO: 94 - protein, 306 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEEIESIKSMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 95 - protein, 306 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEEIVSIKIMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 96 - protein, 306 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEEIQSIKGMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 97 - protein, 306 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHSITSANYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 98 - protein, 306 aa, synthetic construct
IWELKKDVYVVELDWYPDAPGEMVVLTCDTPEEDGITWTLDQSSEVLGSGKTLTIQVKAAGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 99 - protein, 306 aa, synthetic construct
IWELKKDVYVVELDWYPDAPGEMVVLTCDTPEEDGITWTLDQSSEVLGSGKTLTIQVKAAGDAGQYTCHKGGEVLSHSLLLLHAKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 100 - protein, 306 aa, synthetic construct
IWELKKDVYVVELDWYPDAPGEMVVLTCDTPEEDGITWTLDQSSEVLGSGKTLTIQVKAAGDAGQYTCHKGGEVLSHSLLLLHAKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHALKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 101 - protein, 306 aa, synthetic construct
IWELKKDVYVVELDWYAAAPGEMVVLTCDTPEEDGITWTLDQSSEVLGSGKTLTIQVKAAGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 102 - protein, 306 aa, synthetic construct
IWELKKDVYVVELDAYPDAPGEMVVLTCDTPEEDGITWTLDQSSEVLGSGKTLTIQVKEAGDAGQYTCHKGGEVLSHSLLLLHAKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLAYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 103 - protein, 306 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVAALAYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 104 - protein, 306 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLSIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 105 - protein, 306 aa, synthetic construct
IWELKKDVYVVELDAYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 106 - protein, 306 aa, synthetic construct
IWELKKDVYVVELDWYPDAPGEMVVLTCDTPEEDGITWTLDQSSEVLGSGKTLTIQVKEAGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 107 - protein, 306 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHAKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 108 - protein, 306 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHALKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 109 - protein, 306 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLAYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 110 - protein, 159 aa, synthetic construct
RNLPVATPDPGMFPCLHHSQNLLRAVSNMLQKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 111 - protein, 197 aa, synthetic construct
RNLPVATPDPGMFPCLHHSQNLLRAVSNMLQKARQTLKHYSCTSEEIDHEDITKDKTSTVEACLPLELTKNESCLNSRETSFITNGSCLASRKTSFMMALCLSSIYEDLKMYQVEFKTMNAKLLMDPHRQIFLDQNMLAVIDELMQALNFNSETVPQKSSLEEPDPYKTKIKLCILLHAFRIRAVTIDRVMSYLNAS

SEQ ID NO: 112 - protein, 197 aa, synthetic construct
RNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDAAKTKIKLCILLHAFRIRAVTIDRVMSYLNAS

SEQ ID NO: 113 - protein, 197 aa, synthetic construct
RNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDAYKTKIKLCILLHAFRIRAVTIDRVMSYLNAS

SEQ ID NO: 114 - protein, 197 aa, synthetic construct
RNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFAKTKIKLCILLHAFRIRAVTIDRVMSYLNAS

SEQ ID NO: 115 - protein, 518 aa, synthetic construct
RNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASGGGGSGGGGSGGGGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 116 - protein, 518 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 117 - protein, 518 aa, synthetic construct
RNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASGGGGSGGGGSGGGGSIWELKKDVYVVELDAYPDAPGEMVVLTCDTPEEDGITWTLDQSSEVLGSGKTLTIQVKEAGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLAYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 118 - protein, 518 aa, synthetic construct
IWELKKDVYVVELDAYPDAPGEMVVLTCDTPEEDGITWTLDQSSEVLGSGKTLTIQVKEAGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLAYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 119 - protein, 98 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKV

SEQ ID NO: 120 - protein, 12 aa, Homo sapiens
EPKSCDKTHTCP

SEQ ID NO: 121 - protein, 113 aa, Homo sapiens
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAK

SEQ ID NO: 122 - protein, 107 aa, Homo sapiens
GQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 123 - protein, 106 aa, Homo sapiens [sequence omitted]

SEQ ID NO: 124 - protein, 220 aa, Homo sapiens
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 125 - protein, 219 aa, Homo sapiens
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 126 - protein, 223 aa, Homo sapiens
TCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 127 - protein, 222 aa, Homo sapiens
TCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 128 - protein, 232 aa, Homo sapiens
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 129 - protein, 231 aa, Homo sapiens
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 130 - protein, 330 aa, Homo sapiens [sequence omitted]

SEQ ID NO: 131 - protein, 329 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 132 - protein, 98 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRV

SEQ ID NO: 133 - protein, 12 aa, Homo sapiens
ESKYGPPCPSCP

SEQ ID NO: 134 - protein, 110 aa, Homo sapiens
APEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAK

SEQ ID NO: 135 - protein, 107 aa, Homo sapiens
GQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 136 - protein, 106 aa, Homo sapiens
GQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 137 - protein, 217 aa, Homo sapiens
APEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 138 - protein, 216 aa, Homo sapiens
APEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 139 - protein, 223 aa, Homo sapiens
PCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 140 - protein, 222 aa, Homo sapiens
PCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 141 - protein, 229 aa, Homo sapiens
ESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 142 - protein, 228 aa, Homo sapiens
ESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 143 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 144 - protein, 326 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 145 - protein, 107 aa, synthetic construct [sequence omitted]

SEQ ID NO: 146 - protein, 106 aa, synthetic construct
GQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 147 - protein, 220 aa, synthetic construct
PCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 148 - protein, 219 aa, synthetic construct
PCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 149 - protein, 220 aa, synthetic construct
PCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 150 - protein, 219 aa, synthetic construct
PCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 151 - protein, 220 aa, synthetic construct
PCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 152 - protein, 219 aa, synthetic construct
PCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 153 - protein, 223 aa, synthetic construct
TCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 154 - protein, 222 aa, synthetic construct
TCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 155 - protein, 223 aa, synthetic construct
TCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 156 - protein, 222 aa, synthetic construct
TCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 157 - protein, 223 aa, synthetic construct
TCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 158 - protein, 222 aa, synthetic construct
TCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 159 - protein, 232 aa, synthetic construct
EPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 160 - protein, 231 aa, synthetic construct
EPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 161 - protein, 232 aa, synthetic construct; identical to SEQ ID NO: 159

SEQ ID NO: 162 - protein, 231 aa, synthetic construct; identical to SEQ ID NO: 160

SEQ ID NO: 163 - protein, 232 aa, synthetic construct; identical to SEQ ID NO: 159

SEQ ID NO: 164 - protein, 231 aa, synthetic construct; identical to SEQ ID NO: 160

SEQ ID NO: 165 - protein, 217 aa, synthetic construct
APEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 166 - protein, 216 aa, synthetic construct
APEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 167 - protein, 223 aa, synthetic construct
PCPSCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 168 - protein, 222 aa, synthetic construct
PCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 169 - protein, 229 aa, synthetic construct
ESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 170 - protein, 228 aa, synthetic construct
ESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 171 - protein, 230 aa, synthetic construct
AESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 172 - protein, 229 aa, synthetic construct
AESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 173 - protein, 220 aa, synthetic construct
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 174 - protein, 219 aa, synthetic construct
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 175 - protein, 223 aa, synthetic construct
TCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 176 - protein, 222 aa, synthetic construct
TCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 177 - protein, 232 aa, synthetic construct
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 178 - protein, 231 aa, synthetic construct
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 179 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 180 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 181 - protein, 220 aa, synthetic construct
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 182 - protein, 219 aa, synthetic construct
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 183 - protein, 223 aa, synthetic construct
TCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 184 - protein, 222 aa, synthetic construct
TCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 185 - protein, 232 aa, synthetic construct
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 186 - protein, 231 aa, synthetic construct
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 187 - protein, 330 aa, synthetic construct; identical to SEQ ID NO: 179

SEQ ID NO: 188 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 189 - protein, 220 aa, synthetic construct
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 190 - protein, 219 aa, synthetic construct
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 191 - protein, 223 aa, synthetic construct
TCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 192 - protein, 222 aa, synthetic construct
TCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 193 - protein, 232 aa, synthetic construct
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 194 - protein, 231 aa, synthetic construct
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 195 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 196 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 197 - protein, 220 aa, synthetic construct; identical to SEQ ID NO: 189

SEQ ID NO: 198 - protein, 219 aa, synthetic construct; identical to SEQ ID NO: 190

SEQ ID NO: 199 - protein, 223 aa, synthetic construct
TCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 200 - protein, 222 aa, synthetic construct
TCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 201 - protein, 232 aa, synthetic construct
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 202 - protein, 231 aa, synthetic construct
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 203 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 204 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 205 - protein, 220 aa, synthetic construct
PCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 206 - protein, 219 aa, synthetic construct
PCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 207 - protein, 223 aa, synthetic construct
TCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 208 - protein, 222 aa, synthetic construct
TCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 209 - protein, 232 aa, synthetic construct
EPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 210 - protein, 231 aa, synthetic construct
EPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 211 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 212 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 213 - protein, 220 aa, synthetic construct
PCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 214 - protein, 219 aa, synthetic construct
PCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 215 - protein, 223 aa, synthetic construct
TCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 216 - protein, 222 aa, synthetic construct
TCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 217 - protein, 232 aa, synthetic construct
EPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 218 - protein, 231 aa, synthetic construct
EPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 219 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 220 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 221 - protein, 220 aa, synthetic construct
PCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 222 - protein, 219 aa, synthetic construct
PCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 223 - protein, 223 aa, synthetic construct
TCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 224 - protein, 222 aa, synthetic construct
TCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 225 - protein, 232 aa, synthetic construct
EPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 226 - protein, 231 aa, synthetic construct
EPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 227 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 228 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 229 - protein, 220 aa, synthetic construct
PCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 230 - protein, 219 aa, synthetic construct
PCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 231 - protein, 223 aa, synthetic construct
TCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 232 - protein, 222 aa, synthetic construct
TCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 233 - protein, 232 aa, synthetic construct
EPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 234 - protein, 231 aa, synthetic construct
EPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 235 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 236 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 237 - protein, 5 aa, synthetic construct
NYYMS

SEQ ID NO: 238 - protein, 17 aa, synthetic construct
AINSDGGITYYLDTVKG

SEQ ID NO: 239 - protein, 10 aa, synthetic construct
HRSGYFSMDY

SEQ ID NO: 240 - protein, 11 aa, synthetic construct
KASQNVVSAVA

SEQ ID NO: 241 - protein, 7 aa, synthetic construct
SASNRYT

SEQ ID NO: 242 - protein, 9 aa, synthetic construct
QQYSNYPWT

SEQ ID NO: 243 - protein, 449 aa, synthetic construct
EVRLVESGGGLVKPGGSLRLSCAASGFTFSNYYMSWIRQAPEKRLELVSAINSDGGITYYLDTVKGRFTISRDNAKNSLYLQMNSLRAEDTALFYCARHRSGYFSMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 244 - protein, 448 aa, synthetic construct
EVRLVESGGGLVKPGGSLRLSCAASGFTFSNYYMSWIRQAPEKRLELVSAINSDGGITYYLDTVKGRFTISRDNAKNSLYLQMNSLRAEDTALFYCARHRSGYFSMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 245 - protein, 214 aa, synthetic construct
EIVMTQSPATLSVSPGERATLSCKASQNVVSAVAWYQQKPGQSPRLLIYSASNRYTGIPARFSGSGSGTEFTLTISSLQSEDFAAYYCQQYSNYPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 246 - protein, 119 aa, synthetic construct
DVKLVESGGGLVKLGGSLKLSCAASGFTFSNYYMSWVRQTPEKRLELVAAINSDGGITYYLDTVKGRFTISRDNAKNTLYLQMSSLKSEDTALFYCARHRSGYFSMDYWGQGTSVTVSS

SEQ ID NO: 247 - protein, 108 aa, synthetic construct
DIVMTQSQRFMSTTVGDRVSITCKASQNVVSAVAWYQQKPGQSPKLLIYSASNRYTGVPDRFTGSGSGTDFTLTISNMQSEDLADFFCQQYSNYPWTFGGGTKLEIKR

SEQ ID NO: 248 - protein, 449 aa, synthetic construct
DVKLVESGGGLVKLGGSLKLSCAASGFTFSNYYMSWVRQTPEKRLELVAAINSDGGITYYLDTVKGRFTISRDNAKNTLYLQMSSLKSEDTALFYCARHRSGYFSMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 249 - protein, 214 aa, synthetic construct
DIVMTQSQRFMSTTVGDRVSITCKASQNVVSAVAWYQQKPGQSPKLLIYSASNRYTGVPDRFTGSGSGTDFTLTISNMQSEDLADFFCQQYSNYPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 250 - protein, 5 aa, synthetic construct
SHGMA

SEQ ID NO: 251 - protein, 17 aa, synthetic construct
GISNTGRYTNYGSAVKG

SEQ ID NO: 252 - protein, 15 aa, synthetic construct
AAVNCVYGCPGSIDA

SEQ ID NO: 253 - protein, 8 aa, synthetic construct
SGSSGSYG

SEQ ID NO: 254 - protein, 7 aa, synthetic construct
YNDKRPS

SEQ ID NO: 255 - protein, 10 aa, synthetic construct
GSADRSGAGI

SEQ ID NO: 256 - protein, 124 aa, synthetic construct
AVTLDEPGGGLQTPGGTLSLVCKASGFDISSHGMAWVRQAPGKGLEYVAGISNTGRYTNYGSAVKGRATISRDNGQSTVRLQLNDLRAEDAGTYFCARAAVNCVYGCPGSIDAWGLGTEVIVSS

SEQ ID NO: 257 - protein, 103 aa, synthetic construct
ALTQPSSVSANLGETVEITCSGSSGSYGWYQQKSPGSAPVTVIYYNDKRPSDIPSRFSGSKSGSTGTLTITGVQAEDEAVYYCGSADRSGAGIFGAGTTLTVL

SEQ ID NO: 258 - protein, 10 aa, synthetic construct
GFTFTSCYIH

SEQ ID NO: 259 - protein, 18 aa, synthetic construct
WIYPGNGNTKYNEIFKGR

SEQ ID NO: 260 - protein, 10 aa, synthetic construct
GDTTANTMDY

SEQ ID NO: 261 - protein, 11 aa, synthetic construct
RASGNIHNYLA

SEQ ID NO: 262 - protein, 7 aa, synthetic construct
NTITLAD

SEQ ID NO: 263 - protein, 8 aa, synthetic construct
QHFWIPFT

SEQ ID NO: 264 - protein, 118 aa, synthetic construct
QVQLQQSGPELVKPGASVRISCKASGFTFTSCYIHWMKQRPGQGLEWIGWIYPGNGNTKYNEIFKGRATLTTDKSSSTAYMQLSSLTSEDSAVYFCARGDTTANTMDYWGQGTSVTVS

SEQ ID NO: 265 - protein, 106 aa, synthetic construct
DIQMTQSPASLSASVGETVTITCRASGNIHNYLAWYQQKQGKSPQLLVYNTITLADGVPSRFSGSGSGTQYSLKINSLQPEDFGSYYCQHFWIPFTFGAGTKLELK

SEQ ID NO: 266 - protein, 449 aa, synthetic construct
QVQLQQSGPELVKPGASVRISCKASGFTFTSCYIHWMKQRPGQGLEWIGWIYPGNGNTKYNEIFKGRATLTTDKSSSTAYMQLSSLTSEDSAVYFCARGDTTANTMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 267 - protein, 214 aa, synthetic construct
DIQMTQSPASLSASVGETVTITCRASGNIHNYLAWYQQKQGKSPQLLVYNTITLADGVPSRFSGSGSGTQYSLKINSLQPEDFGSYYCQHFWNIPFTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 268 - protein, 10 aa, synthetic construct
GFTFNTYAMY

SEQ ID NO: 269 - protein, 19 aa, synthetic construct
RIRSKSNNYAIYYADSVKD

SEQ ID NO: 270 - protein, 7 aa, synthetic construct
GWDWFAY

SEQ ID NO: 271 - protein, 16 aa, synthetic construct
RSSQSLVHSNGNTYLH

SEQ ID NO: 272 - protein, 7 aa, synthetic construct
KVSNRFS

SEQ ID NO: 273 - protein, 9 aa, synthetic construct
SQNTHVPPT

SEQ ID NO: 274 - protein, 118 aa, synthetic construct
EVQLVESGGRLVQPKGSLKLSCAASGFTFNTYAMYWIRQAPGKGLEWVARIRSKSNNYAIYYADSVKDRFTISRDDSQSMLYLQMNNLKTEDTAMYYCVRGWDWFAYWGQGTPVTVSA

SEQ ID NO: 275 - protein, 112 aa, synthetic construct
DVVMTQTPLSLPVSLGDQASISCRSSQSLVHSNGNTYLHWYLQKPGQSPKLLIYKVSNRFSGVPDRFSGSGSGTDFTLKISRVEAEDLGVYFCSQNTHVPPTFGGGTKLEIK

SEQ ID NO: 276 - protein, 447 aa, synthetic construct
EVQLVESGGRLVQPKGSLKLSCAASGFTFNTYAMYWIRQAPGKGLEWVARIRSKSNNYAIYYADSVKDRFTISRDDSQSMLYLQMNNLKTEDTAMYYCVRGWDWFAYWGQGTPVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 277 - protein, 219 aa, synthetic construct
DVVMTQTPLSLPVSLGDQASISCRSSQSLVHSNGNTYLHWYLQKPGQSPKLLIYKVSNRFSGVPDRFSGSGSGTDFTLKISRVEAEDLGVYFCSQNTHVPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 278 - protein, 10 aa, synthetic construct
GYTFTNYGMN

SEQ ID NO: 279 - protein, 17 aa, synthetic construct
WINTYTGEPTYADDFKG

SEQ ID NO: 280 - protein, 9 aa, synthetic construct
GGIATPTSY

SEQ ID NO: 281 - protein, 16 aa, synthetic construct
KSSQSLLDSDGKTYLN

SEQ ID NO: 282 - protein, 7 aa, synthetic construct
LVSKLDS

SEQ ID NO: 283 - protein, 8 aa, synthetic construct
CQGTHFPW

SEQ ID NO: 284 - protein, 118 aa, synthetic construct
QIQLVQSGPELKKPGETVKISCKASGYTFTNYGMNWVQQAPGKGLKWMGWINTYTGEPTYADDFKGRFAFSLETSASTAYLQINNLKNEDMATYFCARGGIATPTSYWGQGTTLTVSS

SEQ ID NO: 285 - protein, 112 aa, synthetic construct
DVVMTQTPLTLSVTIGQPASISCKSSQSLLDSDGKTYLNWLLQRPGQSPKRLIYLVSKLDSGVPDRFTGSGSGTDFTLKISRVEAEDLGVYYCCQGTHFPWTFGGGTKLEIK

SEQ ID NO: 286 - protein, 447 aa, synthetic construct
QIQLVQSGPELKKPGETVKISCKASGYTFTNYGMNWVQQAPGKGLKWMGWINTYTGEPTYADDFKGRFAFSLETSASTAYLQINNLKNEDMATYFCARGGIATPTSYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 287 - protein, 219 aa, synthetic construct
DVVMTQTPLTLSVTIGQPASISCKSSQSLLDSDGKTYLNWLLQRPGQSPKRLIYLVSKLDSGVPDRFTGSGSGTDFTLKISRVEAEDLGVYYCCQGTHFPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 288 - protein, 6 aa, synthetic construct
LKGKKG

SEQ ID NO: 289 - protein, 7 aa, synthetic construct
LKGKKGC

SEQ ID NO: 290 - protein, 7 aa, synthetic construct
LQVYYRM

SEQ ID NO: 291 - protein, 8 aa, synthetic construct
LQVYYRMC

SEQ ID NO: 292 - protein, 10 aa, synthetic construct
VEPKSCGGGS

SEQ ID NO: 293 - protein, 7 aa, synthetic construct
VQVHYRM

SEQ ID NO: 294 - protein, 8 aa, synthetic construct
YLYLRARV

SEQ ID NO: 295 - protein, 10 aa, synthetic construct
FNRGECGGGS

SEQ ID NO: 296 - protein, 6 aa, synthetic construct
LEGEEG

SEQ ID NO: 297 - protein, 7 aa, synthetic construct
LEGEEGC

SEQ ID NO: 298 - protein, 5 aa, synthetic construct
LGEEG

SEQ ID NO: 299 - protein, 6 aa, synthetic construct
LGEEGC

SEQ ID NO: 300 - protein, 5 aa, synthetic construct
LGKKG

SEQ ID NO: 301 - protein, 6 aa, synthetic construct
LGKKGC

SEQ ID NO: 302 - protein, 28 aa, synthetic construct
EVAALEKEVAALEKEVAALEKEVAALEK

SEQ ID NO: 303 - protein, 28 aa, synthetic construct
KVAALKEKVAALKEKVAALKEKVAALKE

SEQ ID NO: 304 - protein, 770 aa, synthetic construct
EVRLVESGGGLVKPGGSLRLSCAASGFTFSNYYMSWIRQAPEKRLELVSAINSDGGITYYLDTVKGRFTISRDNAKNSLYLQMNSLRAEDTALFYCARHRSGYFSMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 305 - protein, 661 aa, synthetic construct
EVRLVESGGGLVKPGGSLRLSCAASGFTFSNYYMSWIRQAPEKRLELVSAINSDGGITYYLDTVKGRFTISRDNAKNSLYLQMNSLRAEDTALFYCARHRSGYFSMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGKGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 306 - protein, 449 aa, synthetic construct
EVRLVESGGGLVKPGGSLRLSCAASGFTFSNYYMSWIRQAPEKRLELVSAINSDGGITYYLDTVKGRFTISRDNAKNSLYLQMNSLRAEDTALFYCARHRSGYFSMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 307 - protein, 623 aa, synthetic construct
EVRLVESGGGLVKPGGSLRLSCAASGFTFSNYYMSWIRQAPEKRLELVSAINSDGGITYYLDTVKGRFTISRDNAKNSLYLQMNSLRAEDTALFYCARHRSGYFSMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGKGGGGSGGGGSGGGGSRNLPVATPDPGMFPCLHHSQNLLRAVSNMLQKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 308 - protein, 661 aa, synthetic construct
EVRLVESGGGLVKPGGSLRLSCAASGFTFSNYYMSWIRQAPEKRLELVSAINSDGGITYYLDTVKGRFTISRDNAKNSLYLQMNSLRAEDTALFYCARHRSGYFSMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGKGGGGSGGGGSGGGGSRNLPVATPDPGMFPCLHHSQNLLRAVSNMLQKARQTLKHYSCTSEEIDHEDITKDKTSTVEACLPLELTKNESCLNSRETSFITNGSCLASRKTSFMMALCLSSIYEDLKMYQVEFKTMNAKLLMDPHRQIFLDQNMLAVIDELMQALNFNSETVPQKSSLEEPDPYKTKIKLCILLHAFRIRAVTIDRVMSYLNAS

SEQ ID NO: 309 - protein, 661 aa, synthetic construct
EVRLVESGGGLVKPGGSLRLSCAASGFTFSNYYMSWIRQAPEKRLELVSAINSDGGITYYLDTVKGRFTISRDNAKNSLYLQMNSLRAEDTALFYCARHRSGYFSMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGKGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDAAKTKIKLCILLHAFRIRAVTIDRVMSYLNAS

SEQ ID NO: 310 - protein, 661 aa, synthetic construct
EVRLVESGGGLVKPGGSLRLSCAASGFTFSNYYMSWIRQAPEKRLELVSAINSDGGITYYLDTVKGRFTISRDNAKNSLYLQMNSLRAEDTALFYCARHRSGYFSMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGKGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDAYKTKIKLCILLHAFRIRAVTIDRVMSYLNAS

SEQ ID NO: 311 - protein, 661 aa, synthetic construct
EVRLVESGGGLVKPGGSLRLSCAASGFTFSNYYMSWIRQAPEKRLELVSAINSDGGITYYLDTVKGRFTISRDNAKNSLYLQMNSLRAEDTALFYCARHRSGYFSMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGKGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFAKTKIKLCILLHAFRIRAVTIDRVMSYLNAS

SEQ ID NO: 312 - protein, 770 aa, synthetic construct
EVRLVESGGGLVKPGGSLRLSCAASGFTFSNYYMSWIRQAPEKRLELVSAINSDGGITYYLDTVKGRFTISRDNAKNSLYLQMNSLRAEDTALFYCARHRSGYFSMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGASAAEAADAVFTDKTSATVICRKNASISVRAQDRYYSSSWSEWASVPCS

SEQ ID NO: 313 - protein, 665 aa, synthetic construct
EVRLVESGGGLVKPGGSLRLSCAASGFTFSNYYMSWIRQAPEKRLELVSAINSDGGITYYLDTVKGRFTISRDNAKNSLYLQMNSLRAEDTALFYCARHRSGYFSMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 314 - protein, 649 aa, synthetic construct
EVRLVESGGGLVKPGGSLRLSCAASGFTFSNYYMSWIRQAPEKRLELVSAINSDGGITYYLDTVKGRFTISRDNAKNSLYLQMNSLRAEDTALFYCARHRSGYFSMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEEIES

SEQ ID NO: 315 - protein, 770 aa, synthetic construct
EVRLVESGGGLVKPGGSLRLSCAASGFTFSNYYMSWIRQAPEKRLELVSAINSDGGITYYLDTVKGRFTISRDNAKNSLYLQMNSLRAEDTALFYCARHRSGYFSMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSIWELKKDVYVVELDWYPDAPGEMVVLTCDTPEEDGITWTLDQSSEVLGSGKTLTIQVKKFGDAGQYTCHKGGEVLSHSLLLLHKKEDGIWSTDILKDQEEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKEDRVFTDKTSATVICRKNASISVRAQDRYYSSSWSEWASVPCS

SEQ ID NO: 316 - protein, 770 aa, synthetic construct
EVRLVESGGGLVKPGGSLRLSCAASGFTFSNYYMSWIRQAPEKRLELVSAINSDGGITYYLDTVKGRFTISRDNAKNSLYLQMNSLRAEDTALFYCARHRSGYFSMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEEIESIKSMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 317 - protein, 770 aa, synthetic construct
EVRLVESGGGLVKPGGSLRLSCAASGFTFSNYYMSWIRQAPEKRLELVSAINSDGGITYYLDTVKGRFTISRDNAKNSLYLQMNSLRAEDTALFYCARHRSGYFSMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEEIVSIKIMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 318 - protein, 770 aa, synthetic construct
EVRLVESGGGLVKPGGSLRLSCAASGFTFSNYYMSWIRQAPEKRLELVSAINSDGGITYYLDTVKGRFTISRDNAKNSLYLQMNSLRAEDTALFYCARHRSGYFSMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEEIQSIKGMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 319 - protein, 769 aa, synthetic construct
EVRLVESGGGLVKPGGSLRLSCAASGFTFSNYYMSWIRQAPEKRLELVSAINSDGGITYYLDTVKGRFTISRDNAKNSLYLQMNSLRAEDTALFYCARHRSGYFSMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHIKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 320 - protein, 770 aa, synthetic construct
EVRLVESGGGLVKPGGSLRLSCAASGFTFSNYYMSWIRQAPEKRLELVSAINSDGGITYYLDTVKGRFTISRDNAKNSLYLQMNSLRAEDTALFYCARHRSGYFSMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHSITSANYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 321 - protein, 770 aa, synthetic construct
EVRLVESGGGLVKPGGSLRLSCAASGFTFSNYYMSWIRQAPEKRLELVSAINSDGGITYYLDTVKGRFTISRDNAKNSLYLQMNSLRAEDTALFYCARHRSGYFSMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSIWELKKDVYVVELDWYPDAPGEMVVLTCDTPEEDGITWTLDQSSEVLGSGKTLTIQVKAAGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 322 - protein, 770 aa, synthetic construct
EVRLVESGGGLVKPGGSLRLSCAASGFTFSNYYMSWIRQAPEKRLELVSAINSDGGITYYLDTVKGRFTISRDNAKNSLYLQMNSLRAEDTALFYCARHRSGYFSMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSIWELKKDVYVVELDWYPDAPGEMVVLTCDTPEEDGITWTLDQSSEVLGSGKTLTIQVKAAGDAGQYTCHKGGEVLSHSLLLLHAKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 323 - protein, 770 aa, synthetic construct
EVRLVESGGGLVKPGGSLRLSCAASGFTFSNYYMSWIRQAPEKRLELVSAINSDGGITYYLDTVKGRFTISRDNAKNSLYLQMNSLRAEDTALFYCARHRSGYFSMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSIWELKKDVYVVELDWYPDAPGEMVVLTCDTPEEDGITWTLDQSSEVLGSGKTLTIQVKAAGDAGQYTCHKGGEVLSHSLLLLHAKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHALKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 324 - protein, 770 aa, synthetic construct
EVRLVESGGGLVKPGGSLRLSCAASGFTFSNYYMSWIRQAPEKRLELVSAINSDGGITYYLDTVKGRFTISRDNAKNSLYLQMNSLRAEDTALFYCARHRSGYFSMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSIWELKKDVYVVELDWYAAAPGEMVVLTCDTPEEDGITWTLDQSSEVLGSGKTLTIQVKAAGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 325 - protein, 770 aa, synthetic construct
EVRLVESGGGLVKPGGSLRLSCAASGFTFSNYYMSWIRQAPEKRLELVSAINSDGGITYYLDTVKGRFTISRDNAKNSLYLQMNSLRAEDTALFYCARHRSGYFSMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSIWELKKDVYVVELDAYPDAPGEMVVLTCDTPEEDGITWTLDQSSEVLGSGKTLTIQVKEAGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHALKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 326 - protein, 770 aa, synthetic construct
EVRLVESGGGLVKPGGSLRLSCAASGFTFSNYYMSWIRQAPEKRLELVSAINSDGGITYYLDTVKGRFTISRDNAKNSLYLQMNSLRAEDTALFYCARHRSGYFSMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSIWELKKDVYVVELDAYPDAPGEMVVLTCDTPEEDGITWTLDQSSEVLGSGKTLTIQVKEAGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLAYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 327 - protein, 770 aa, synthetic construct
EVRLVESGGGLVKPGGSLRLSCAASGFTFSNYYMSWIRQAPEKRLELVSAINSDGGITYYLDTVKGRFTISRDNAKNSLYLQMNSLRAEDTALFYCARHRSGYFSMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSIWELKKDVYVVELDWYPDAPGEMVVLTCDTPEEDGITWTLDQSSEVLGSGKTLTIQVKEFGDAGQYTCHKGGEVLSHSLLLLHAKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHALAYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 328 - protein, 770 aa, synthetic construct
EVRLVESGGGLVKPGGSLRLSCAASGFTFSNYYMSWIRQAPEKRLELVSAINSDGGITYYLDTVKGRFTISRDNAKNSLYLQMNSLRAEDTALFYCARHRSGYFSMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSIWELKKDVYVVELDAYPDAPGEMVVLTCDTPEEDGITWTLDQSSEVLGSGKTLTIQVKEAGDAGQYTCHKGGEVLSHSLLLLHAKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLAYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 329 - protein, 770 aa, synthetic construct
EVRLVESGGGLVKPGGSLRLSCAASGFTFSNYYMSWIRQAPEKRLELVSAINSDGGITYYLDTVKGRFTISRDNAKNSLYLQMNSLRAEDTALFYCARHRSGYFSMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVAALAYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 330 - protein, 770 aa, synthetic construct
EVRLVESGGGLVKPGGSLRLSCAASGFTFSNYYMSWIRQAPEKRLELVSAINSDGGITYYLDTVKGRFTISRDNAKNSLYLQMNSLRAEDTALFYCARHRSGYFSMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSIWELKKDVYVVELDAYPDAPGEMVVLTCDTPEEDGITWTLDQSSEVLGSGKTLTIQVKEAGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 331 - protein, 770 aa, synthetic construct
EVRLVESGGGLVKPGGSLRLSCAASGFTFSNYYMSWIRQAPEKRLELVSAINSDGGITYYLDTVKGRFTISRDNAKNSLYLQMNSLRAEDTALFYCARHRSGYFSMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSIWELKKDVYVVELDAYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHALKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 332 - protein, 770 aa, synthetic construct
EVRLVESGGGLVKPGGSLRLSCAASGFTFSNYYMSWIRQAPEKRLELVSAINSDGGITYYLDTVKGRFTISRDNAKNSLYLQMNSLRAEDTALFYCARHRSGYFSMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSIWELKKDVYVVELDAYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLAYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 333 - protein, 770 aa, synthetic construct
EVRLVESGGGLVKPGGSLRLSCAASGFTFSNYYMSWIRQAPEKRLELVSAINSDGGITYYLDTVKGRFTISRDNAKNSLYLQMNSLRAEDTALFYCARHRSGYFSMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSIWELKKDVYVVELDAYPDAPGEMVVLTCDTPEEDGITWTLDQSSEVLGSGKTLTIQVKEFGDAGQYTCHKGGEVLSHSLLLLHAKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 334 - protein, 770 aa, synthetic construct
EVRLVESGGGLVKPGGSLRLSCAASGFTFSNYYMSWIRQAPEKRLELVSAINSDGGITYYLDTVKGRFTISRDNAKNSLYLQMNSLRAEDTALFYCARHRSGYFSMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSIWELKKDVYVVELDWYPDAPGEMVVLTCDTPEEDGITWTLDQSSEVLGSGKTLTIQVKEAGDAGQYTCHKGGEVLSHSLLLLHAKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 335 - protein, 770 aa, synthetic construct
EVRLVESGGGLVKPGGSLRLSCAASGFTFSNYYMSWIRQAPEKRLELVSAINSDGGITYYLDTVKGRFTISRDNAKNSLYLQMNSLRAEDTALFYCARHRSGYFSMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSIWELKKDVYVVELDWYPDAPGEMVVLTCDTPEEDGITWTLDQSSEVLGSGKTLTIQVKEAGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHALKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 336 - protein, 770 aa, synthetic construct
EVRLVESGGGLVKPGGSLRLSCAASGFTFSNYYMSWIRQAPEKRLELVSAINSDGGITYYLDTVKGRFTISRDNAKNSLYLQMNSLRAEDTALFYCARHRSGYFSMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSIWELKKDVYVVELDWYPDAPGEMVVLTCDTPEEDGITWTLDQSSEVLGSGKTLTIQVKEAGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLAYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 337 - protein, 770 aa, synthetic construct
EVRLVESGGGLVKPGGSLRLSCAASGFTFSNYYMSWIRQAPEKRLELVSAINSDGGITYYLDTVKGRFTISRDNAKNSLYLQMNSLRAEDTALFYCARHRSGYFSMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHALAYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 338 - protein, 769 aa, synthetic construct
VRLVESGGGLVKPGGSLRLSCAASGFTFSNYYMSWIRQAPEKRLELVSAINSDGGITYYLDTVKGRFTISRDNAKNSLYLQMNSLRAEDTALFYCARHRSGYFSMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSIWELKKDVYVVELDWYPDAPGEMVVLTCDTPEEDGITWTLDQSSEVLGSGKTLTIQVKEFGDAGQYTCHKGGEVLSHSLLLLHAKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHALKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 339 - protein, 770 aa, synthetic construct
EVRLVESGGGLVKPGGSLRLSCAASGFTFSNYYMSWIRQAPEKRLELVSAINSDGGITYYLDTVKGRFTISRDNAKNSLYLQMNSLRAEDTALFYCARHRSGYFSMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSIWELKKDVYVVELDWYPDAPGEMVVLTCDTPEEDGITWTLDQSSEVLGSGKTLTIQVKEFGDAGQYTCHKGGEVLSHSLLLLHAKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLAYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 340 - protein, 770 aa, synthetic construct
EVRLVESGGGLVKPGGSLRLSCAASGFTFSNYYMSWIRQAPEKRLELVSAINSDGGITYYLDTVKGRFTISRDNAKNSLYLQMNSLRAEDTALFYCARHRSGYFSMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSIWELKKDVYVVELDAYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 341 - protein, 770 aa, synthetic construct
EVRLVESGGGLVKPGGSLRLSCAASGFTFSNYYMSWIRQAPEKRLELVSAINSDGGITYYLDTVKGRFTISRDNAKNSLYLQMNSLRAEDTALFYCARHRSGYFSMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSIWELKKDVYVVELDWYPDAPGEMVVLTCDTPEEDGITWTLDQSSEVLGSGKTLTIQVKEAGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 342 - protein, 770 aa, synthetic construct
EVRLVESGGGLVKPGGSLRLSCAASGFTFSNYYMSWIRQAPEKRLELVSAINSDGGITYYLDTVKGRFTISRDNAKNSLYLQMNSLRAEDTALFYCARHRSGYFSMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSIWELKKDVYVVELDWYPDAPGEMVVLTCDTPEEDGITWTLDQSSEVLGSGKTLTIQVKEFGDAGQYTCHKGGEVLSHSLLLLHAKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 343 - protein, 770 aa, synthetic construct
EVRLVESGGGLVKPGGSLRLSCAASGFTFSNYYMSWIRQAPEKRLELVSAINSDGGITYYLDTVKGRFTISRDNAKNSLYLQMNSLRAEDTALFYCARHRSGYFSMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHALKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 344 - protein, 770 aa, synthetic construct
EVRLVESGGGLVKPGGSLRLSCAASGFTFSNYYMSWIRQAPEKRLELVSAINSDGGITYYLDTVKGRFTISRDNAKNSLYLQMNSLRAEDTALFYCARHRSGYFSMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLAYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 345 - protein, 974 aa, synthetic construct
EVRLVESGGGLVKPGGSLRLSCAASGFTFSNYYMSWIRQAPEKRLELVSAINSDGGITYYLDTVKGRFTISRDNAKNSLYLQMNSLRAEDTALFYCARHRSGYFSMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASGGGGGGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 346 - protein, 936 aa, synthetic construct
EVRLVESGGGLVKPGGSLRLSCAASGFTFSNYYMSWIRQAPEKRLELVSAINSDGGITYYLDTVKGRFTISRDNAKNSLYLQMNSLRAEDTALFYCARHRSGYFSMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSRNLPVATPDPGMFPCLHHSQNLLRAVSNMLQKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASGGGGGGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 347 - protein, 974 aa, synthetic construct
EVRLVESGGGLVKPGGSLRLSCAASGFTFSNYYMSWIRQAPEKRLELVSAINSDGGITYYLDTVKGRFTISRDNAKNSLYLQMNSLRAEDTALFYCARHRSGYFSMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSRNLPVATPDPGMFPCLHHSQNLLRAVSNMLQKARQTLKHYSCTSEEIDHEDITKDKTSTVEACLPLELTKNESCLNSRETSFITNGSCLASRKTSFMMALCLSSIYEDLKMYQVEFKTMNAKLLMDPHRQIFLDQNMLAVIDELMQALNFNSETVPQKSSLEEPDPYKTKIKLCILLHAFRIRAVTIDRVMSYLNASGGGGGGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 348 - protein, 974 aa, synthetic construct
EVRLVESGGGLVKPGGSLRLSCAASGFTFSNYYMSWIRQAPEKRLELVSAINSDGGITYYLDTVKGRFTISRDNAKNSLYLQMNSLRAEDTALFYCARHRSGYFSMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDAAKTKIKLCILLHAFRIRAVTIDRVMSYLNASGGGGGGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 349 - protein, 974 aa, synthetic construct
EVRLVESGGGLVKPGGSLRLSCAASGFTFSNYYMSWIRQAPEKRLELVSAINSDGGITYYLDTVKGRFTISRDNAKNSLYLQMNSLRAEDTALFYCARHRSGYFSMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDAYKTKIKLCILLHAFRIRAVTIDRVMSYLNASGGGGGGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 350 - protein, 974 aa, synthetic construct
EVRLVESGGGLVKPGGSLRLSCAASGFTFSNYYMSWIRQAPEKRLELVSAINSDGGITYYLDTVKGRFTISRDNAKNSLYLQMNSLRAEDTALFYCARHRSGYFSMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFAKTKIKLCILLHAFRIRAVTIDRVMSYLNASGGGGGGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 351 - protein, 974 aa, synthetic construct
EVRLVESGGGLVKPGGSLRLSCAASGFTFSNYYMSWIRQAPEKRLELVSAINSDGGITYYLDTVKGRFTISRDNAKNSLYLQMNSLRAEDTALFYCARHRSGYFSMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASGGGGGGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGASAAEAADAVFTDKTSATVICRKNASISVRAQDRYYSSSWSEWASVPCS

SEQ ID NO: 352 - protein, 869 aa, synthetic construct
EVRLVESGGGLVKPGGSLRLSCAASGFTFSNYYMSWIRQAPEKRLELVSAINSDGGITYYLDTVKGRFTISRDNAKNSLYLQMNSLRAEDTALFYCARHRSGYFSMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASGGGGGGSFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 353 - protein, 853 aa, synthetic construct
EVRLVESGGGLVKPGGSLRLSCAASGFTFSNYYMSWIRQAPEKRLELVSAINSDGGITYYLDTVKGRFTISRDNAKNSLYLQMNSLRAEDTALFYCARHRSGYFSMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASGGGGGGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEEIES

SEQ ID NO: 354 - protein, 974 aa, synthetic construct
EVRLVESGGGLVKPGGSLRLSCAASGFTFSNYYMSWIRQAPEKRLELVSAINSDGGITYYLDTVKGRFTISRDNAKNSLYLQMNSLRAEDTALFYCARHRSGYFSMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASGGGGGGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLSIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 355 - protein, 974 aa, synthetic construct
EVRLVESGGGLVKPGGSLRLSCAASGFTFSNYYMSWIRQAPEKRLELVSAINSDGGITYYLDTVKGRFTISRDNAKNSLYLQMNSLRAEDTALFYCARHRSGYFSMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASGGGGGGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEEIESIKSMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 356 - protein, 974 aa, synthetic construct
EVRLVESGGGLVKPGGSLRLSCAASGFTFSNYYMSWIRQAPEKRLELVSAINSDGGITYYLDTVKGRFTISRDNAKNSLYLQMNSLRAEDTALFYCARHRSGYFSMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASGGGGGGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEEIVSIKIMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 357 - protein, 974 aa, synthetic construct
EVRLVESGGGLVKPGGSLRLSCAASGFTFSNYYMSWIRQAPEKRLELVSAINSDGGITYYLDTVKGRFTISRDNAKNSLYLQMNSLRAEDTALFYCARHRSGYFSMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASGGGGGGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEEIQSIKGMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 358 - protein, 973 aa, synthetic construct
EVRLVESGGGLVKPGGSLRLSCAASGFTFSNYYMSWIRQAPEKRLELVSAINSDGGITYYLDTVKGRFTISRDNAKNSLYLQMNSLRAEDTALFYCARHRSGYFSMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASGGGGGGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHIKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 359 - protein, 974 aa, synthetic construct
EVRLVESGGGLVKPGGSLRLSCAASGFTFSNYYMSWIRQAPEKRLELVSAINSDGGITYYLDTVKGRFTISRDNAKNSLYLQMNSLRAEDTALFYCARHRSGYFSMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASGGGGGGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHSITSANYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 360 - protein, 974 aa, synthetic construct
EVRLVESGGGLVKPGGSLRLSCAASGFTFSNYYMSWIRQAPEKRLELVSAINSDGGITYYLDTVKGRFTISRDNAKNSLYLQMNSLRAEDTALFYCARHRSGYFSMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASGGGGGGSIWELKKDVYVVELDWYPDAPGEMVVLTCDTPEEDGITWTLDQSSEVLGSGKTLTIQVKAAGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 361 - protein, 974 aa, synthetic construct
EVRLVESGGGLVKPGGSLRLSCAASGFTFSNYYMSWIRQAPEKRLELVSAINSDGGITYYLDTVKGRFTISRDNAKNSLYLQMNSLRAEDTALFYCARHRSGYFSMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASGGGGGGSIWELKKDVYVVELDWYPDAPGEMVVLTCDTPEEDGITWTLDQSSEVLGSGKTLTIQVKAAGDAGQYTCHKGGEVLSHSLLLLHAKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 362 - protein, 974 aa, synthetic construct
EVRLVESGGGLVKPGGSLRLSCAASGFTFSNYYMSWIRQAPEKRLELVSAINSDGGITYYLDTVKGRFTISRDNAKNSLYLQMNSLRAEDTALFYCARHRSGYFSMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASGGGGGGSIWELKKDVYVVELDWYPDAPGEMVVLTCDTPEEDGITWTLDQSSEVLGSGKTLTIQVKAAGDAGQYTCHKGGEVLSHSLLLLHAKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHALKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 363 - protein, 974 aa, synthetic construct
EVRLVESGGGLVKPGGSLRLSCAASGFTFSNYYMSWIRQAPEKRLELVSAINSDGGITYYLDTVKGRFTISRDNAKNSLYLQMNSLRAEDTALFYCARHRSGYFSMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASGGGGGGSIWELKKDVYVVELDWYAAAPGEMVVLTCDTPEEDGITWTLDQSSEVLGSGKTLTIQVKAAGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 364 - protein, 974 aa, synthetic construct
EVRLVESGGGLVKPGGSLRLSCAASGFTFSNYYMSWIRQAPEKRLELVSAINSDGGITYYLDTVKGRFTISRDNAKNSLYLQMNSLRAEDTALFYCARHRSGYFSMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASGGGGGGSIWELKKDVYVVELDAYPDAPGEMVVLTCDTPEEDGITWTLDQSSEVLGSGKTLTIQVKEAGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHALKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 365 - protein, 974 aa, synthetic construct
EVRLVESGGGLVKPGGSLRLSCAASGFTFSNYYMSWIRQAPEKRLELVSAINSDGGITYYLDTVKGRFTISRDNAKNSLYLQMNSLRAEDTALFYCARHRSGYFSMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASGGGGGGSIWELKKDVYVVELDAYPDAPGEMVVLTCDTPEEDGITWTLDQSSEVLGSGKTLTIQVKEAGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLAYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 366 - protein, 974 aa, synthetic construct
EVRLVESGGGLVKPGGSLRLSCAASGFTFSNYYMSWIRQAPEKRLELVSAINSDGGITYYLDTVKGRFTISRDNAKNSLYLQMNSLRAEDTALFYCARHRSGYFSMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASGGGGGGSIWELKKDVYVVELDWYPDAPGEMVVLTCDTPEEDGITWTLDQSSEVLGSGKTLTIQVKEFGDAGQYTCHKGGEVLSHSLLLLHAKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHALAYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 367 - protein, 974 aa, synthetic construct
EVRLVESGGGLVKPGGSLRLSCAASGFTFSNYYMSWIRQAPEKRLELVSAINSDGGITYYLDTVKGRFTISRDNAKNSLYLQMNSLRAEDTALFYCARHRSGYFSMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASGGGGGGSIWELKKDVYVVELDAYPDAPGEMVVLTCDTPEEDGITWTLDQSSEVLGSGKTLTIQVKEAGDAGQYTCHKGGEVLSHSLLLLHAKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLAYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 368 - protein, 974 aa, synthetic construct
EVRLVESGGGLVKPGGSLRLSCAASGFTFSNYYMSWIRQAPEKRLELVSAINSDGGITYYLDTVKGRFTISRDNAKNSLYLQMNSLRAEDTALFYCARHRSGYFSMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASGGGGGGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVAALAYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 369 - protein, 7 aa, synthetic construct
GGGGGGS

SEQ ID NO: 370 - protein, 789 aa, synthetic construct
MMSFVSLLLVGILFHATQAEVRLVESGGGLVKPGGSLRLSCAASGFTFSNYYMSWIRQAPEKRLELVSAINSDGGITYYLDTVKGRFTISRDNAKNSLYLQMNSLRAEDTALFYCARHRSGYFSMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSIWELKKDVYVVELDAYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 371 - protein, 680 aa, synthetic construct
MMSFVSLLLVGILFHATQAEVRLVESGGGLVKPGGSLRLSCAASGFTFSNYYMSWIRQAPEKRLELVSAINSDGGITYYLDTVKGRFTISRDNAKNSLYLQMNSLRAEDTALFYCARHRSGYFSMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGKGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 372 - protein, 233 aa, synthetic construct
MMSFVSLLLVGILFHATQAEIVMTQSPATLSVSPGERATLSCKASQNVVSAVAWYQQKPGQSPRLLIYSASNRYTGIPARFSGSGSGTEFTLTISSLQSEDFAAYYCQQYRNYPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 373 - protein, 789 aa, synthetic construct
MMSFVSLLLVGILFHATQAEVRLVESGGGLVKPGGSLRLSCAASGFTFSNYYMSWIRQAPEKRLELVSAINSDGGITYYLDTVKGRFTISRDNAKNSLYLQMNSLRAEDTALFYCARHRSGYFSMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSIWELKKDVYVVELDWYPDAPGEMVVLTCDTPEEDGITWTLDQSSEVLGSGKTLTIQVKEAGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 374 - protein, 789 aa, synthetic construct
MMSFVSLLLVGILFHATQAEVRLVESGGGLVKPGGSLRLSCAASGFTFSNYYMSWIRQAPEKRLELVSAINSDGGITYYLDTVKGRFTISRDNAKNSLYLQMNSLRAEDTALFYCARHRSGYFSMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHALKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 375 - protein, 789 aa, synthetic construct
MMSFVSLLLVGILFHATQAEVRLVESGGGLVKPGGSLRLSCAASGFTFSNYYMSWIRQAPEKRLELVSAINSDGGITYYLDTVKGRFTISRDNAKNSLYLQMNSLRAEDTALFYCARHRSGYFSMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLAYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 376 - protein, 789 aa, synthetic construct
MMSFVSLLLVGILFHATQAEVRLVESGGGLVKPGGSLRLSCAASGFTFSNYYMSWIRQAPEKRLELVSAINSDGGITYYLDTVKGRFTISRDNAKNSLYLQMNSLRAEDTALFYCARHRSGYFSMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSIWELKKDVYVVELDAYPDAPGEMVVLTCDTPEEDGITWTLDQSSEVLGSGKTLTIQVKEAGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 377 - protein, 789 aa, synthetic construct
MMSFVSLLLVGILFHATQAEVRLVESGGGLVKPGGSLRLSCAASGFTFSNYYMSWIRQAPEKRLELVSAINSDGGITYYLDTVKGRFTISRDNAKNSLYLQMNSLRAEDTALFYCARHRSGYFSMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSIWELKKDVYVVELDAYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHALKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 378 - protein, 789 aa, synthetic construct
MMSFVSLLLVGILFHATQAEVRLVESGGGLVKPGGSLRLSCAASGFTFSNYYMSWIRQAPEKRLELVSAINSDGGITYYLDTVKGRFTISRDNAKNSLYLQMNSLRAEDTALFYCARHRSGYFSMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSIWELKKDVYVVELDAYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLAYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 379 - protein, 789 aa, synthetic construct
MMSFVSLLLVGILFHATQAEVRLVESGGGLVKPGGSLRLSCAASGFTFSNYYMSWIRQAPEKRLELVSAINSDGGITYYLDTVKGRFTISRDNAKNSLYLQMNSLRAEDTALFYCARHRSGYFSMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSIWELKKDVYVVELDWYPDAPGEMVVLTCDTPEEDGITWTLDQSSEVLGSGKTLTIQVKEAGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHALKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 380 - protein, 789 aa, synthetic construct
MMSFVSLLLVGILFHATQAEVRLVESGGGLVKPGGSLRLSCAASGFTFSNYYMSWIRQAPEKRLELVSAINSDGGITYYLDTVKGRFTISRDNAKNSLYLQMNSLRAEDTALFYCARHRSGYFSMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSIWELKKDVYVVELDWYPDAPGEMVVLTCDTPEEDGITWTLDQSSEVLGSGKTLTIQVKEAGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLAYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 381 - protein, 789 aa, synthetic construct
MMSFVSLLLVGILFHATQAEVRLVESGGGLVKPGGSLRLSCAASGFTFSNYYMSWIRQAPEKRLELVSAINSDGGITYYLDTVKGRFTISRDNAKNSLYLQMNSLRAEDTALFYCARHRSGYFSMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHALAYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 382 - protein, 789 aa, synthetic construct
MMSFVSLLLVGILFHATQAEVRLVESGGGLVKPGGSLRLSCAASGFTFSNYYMSWIRQAPEKRLELVSAINSDGGITYYLDTVKGRFTISRDNAKNSLYLQMNSLRAEDTALFYCARHRSGYFSMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSIWELKKDVYVVELDAYPDAPGEMVVLTCDTPEEDGITWTLDQSSEVLGSGKTLTIQVKEAGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLAYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 383 - protein, 789 aa, synthetic construct
MMSFVSLLLVGILFHATQAEVRLVESGGGLVKPGGSLRLSCAASGFTFSNYYMSWIRQAPEKRLELVSAINSDGGITYYLDTVKGRFTISRDNAKNSLYLQMNSLRAEDTALFYCARHRSGYFSMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 384 - protein, 572 aa, synthetic construct
MMSFVSLLLVGILFHATQAEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 385 - protein, 463 aa, synthetic construct
MMSFVSLLLVGILFHATQAEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGKGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 386 - protein, 572 aa, synthetic construct
MMSFVSLLLVGILFHATQAEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSIWELKKDVYVVELDWYPDAPGEMVVLTCDTPEEDGITWTLDQSSEVLGSGKTLTIQVKKFGDAGQYTCHKGGEVLSHSLLLLHKKEDGIWSTDILKDQEEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKEDRVFTDKTSATVICRKNASISVRAQDRYYSSSWSEWASVPCS

SEQ ID NO: 387 - protein, 463 aa, synthetic construct; identical to SEQ ID NO: 385

SEQ ID NO: 388 - protein, 468 aa, synthetic construct
MMSFVSLLLVGILFHATQAEVRLVESGGGLVKPGGSLRLSCAASGFTFSNYYMSWIRQAPEKRLELVSAINSDGGITYYLDTVKGRFTISRDNAKNSLYLQMNSLRAEDTALFYCARHRSGYFSMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 389 - protein, 468 aa, synthetic construct
MMSFVSLLLVGILFHATQAEVRLVESGGGLVKPGGSLRLSCAASGFTFSNYYMSWIRQAPEKRLELVSAINSDGGITYYLDTVKGRFTISRDNAKNSLYLQMNSLRAEDTALFYCARHRSGYFSMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 390 - protein, 803 aa, synthetic construct
MMSFVSLLLVGILFHATQAQVQLQQSGPGPAKPSQTLSLTCAISGDSVSSDSAAWNWIRQSPSRGLEWLGRTYYRSTWYRDYAPSVKSRITINPDTSKNQFSLQLNSVTPEDTAVYYCARDKDSFESNGSLYTAKKMGFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 391 - protein, 694 aa, synthetic construct
MMSFVSLLLVGILFHATQAQVQLQQSGPGPAKPSQTLSLTCAISGDSVSSDSAAWNWIRQSPSRGLEWLGRTYYRSTWYRDYAPSVKSRITINPDTSKNQFSLQLNSVTPEDTAVYYCARDKDSFESNGSLYTAKKMGFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGKGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 392 - protein, 234 aa, synthetic construct
MMSFVSLLLVGILFHATQAETTLTQSPGTLSLSPGERATLSCRASQSVRSSYLAWYQQKPGQAPRLLIYGASSRATGIPERFSGSGSGTDFTLTISRLEPEDFAVYYCQQYGSSPITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 393 - protein, 803 aa, synthetic construct
MMSFVSLLLVGILFHATQAQVQLQQSGPGPAKPSQTLSLTCAISGDSVSSDSAAWNWIRQSPSRGLEWLGRTYYRSTWYRDYAPSVKSRITINPDTSKNQFSLQLNSVTPEDTAVYYCARDKDSFESNGSLYTAKKMGFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSIWELKKDVYVVELDAYPDAPGEMVVLTCDTPEEDGITWTLDQSSEVLGSGKTLTIQVKEAGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLAYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 394 - protein, 22 aa, Homo sapiens
MCPARSLLLVATLVLLDHLSLA

(19) United States
(12) Patent Application Publication
(10) Pub. No.: US 2024/0392031 A1
(43) Pub. Date: Nov. 28, 2024

(54) CAIX TARGETING IL-12 FUSION PROTEINS AND METHODS OF USE THEREOF

(71) Applicant: BICARA THERAPEUTICS INC., Boston, MA (US)

(72) Inventors: Jaya BHATNAGAR, Maharashtra (IN); Arvind Vittal GOSWAMI, Bangalore (IN); Harish Kumar TRIPURANA, Karnataka (IN); Pradip NAIR, Bangalore (IN); Ramakrishnan Melarkode SUBBARAMAN, Bangalore (IN); Shiv Ram KRISHN, Rajasthan (IN); Srinivas Reddy BOREDDY, Karnataka (IN); Reshmi NAIR, Hobli (IN); Avanish K. VARSHNEY, Westford, MA (US); Seng-Lai TAN, Dallas, TX (US)

(21) Appl. No.: 18/607,049

(22) Filed: Mar. 15, 2024

Related U.S. Application Data
(63) Continuation of application No. PCT/US22/43762, filed on Sep. 16, 2022.
(60) Provisional application No. 63/245,523, filed on Sep. 17, 2021.

(51) Int. Cl. C07K 16/30; A61P 35/00; C07K 14/54; G01N 33/68

(52) U.S. Cl. CPC C07K 16/30; A61P 35/00; C07K 14/5434; G01N 33/6893; C07K 2317/522; C07K 2317/524; C07K 2317/526; C07K 2317/56; C07K 2319/00; G01N 2333/988

(57) ABSTRACT

Provided herein are IL-12p40 and IL-12p35 polypeptides and compositions (e.g., pharmaceutical compositions) comprising the same; as well as methods of making the IL-12p40 and IL-12p35 polypeptides and compositions. Further provided herein are fusion proteins (e.g., antibody fusion proteins) that comprise an IL-12p40 polypeptide (e.g., an IL-12p40 polypeptide described herein) and/or IL-12p35 polypeptide (e.g., an IL-12p35 polypeptide described herein). The IL-12p40 polypeptides, IL-12p35 polypeptides, and fusion proteins provided herein are useful in pharmaceutical compositions and methods of treating diseases (e.g., cancer).

### CROSS REFERENCE TO RELATED APPLICATION

This application is a continuation of International Application No.; PCT/US2022/043762 filed Sep. 16, 2022, which claims priority to and the benefit of U.S. Provisional Application No. 63/245,523, filed Sep. 17, 2021, the entire contents of each of which are incorporated herein by reference.

### SEQUENCE LISTING

The instant application contains a Sequence Listing which has been submitted electronically in XML format and is hereby incorporated by reference in its entirety. Said XML copy, created on Jul. 8, 2024, is named 62804_31US01_SL.xml and is 469,158 bytes in size.

### 1. FIELD

This disclosure relates to IL-12p40 variants, IL-12p35 variants, IL-12 fusion proteins, and methods of use thereof. This disclosure further relates to multispecific IL-12 fusion proteins that target IL-12 to cells expressing a target protein on the cell surface (e.g., hCAIX), and methods of using the same.

### 2. BACKGROUND

Human IL-12 (hIL-12) is a pleotropic secreted cytokine composed of an a subunit, human IL-12p35 (hIL-12p35), and a β subunit, human IL-12p40 (hIL-12p40). The naturally occurring hIL-12p35 and hIL-12p40 subunits are linked through a disulfide bond to form the bioactive hIL12-p70 cytokine. hIL-12 is, inter alia, pro-inflammatory, and mediates its functions through binding to the hIL-12 receptor (hIL-12R). The high affinity hIL-12R is heterodimeric comprising a hIL-12Rβ1 subunit and a hIL-12Rβ2 subunit. The hIL-12R is expressed in a constitutive or inducible manner in a variety of immune cells, including natural killer (NK) cells, T-cells, and B-cells. Binding of hIL-12 to the hIL-12R expressed on e.g., activated T cells, NK cells, and dendritic cells, activates the TYK2, JAK2, and STAT signaling pathways. One of the principal roles of hIL-12 is the activation of T-cells and NK cells, leading to increased production of INF-γ, proliferation, and cytotoxic potential.

### 3. SUMMARY

Provided herein are, inter alia, IL-12p40 variant and IL-12p35 polypeptides and polynucleotides encoding the same; IL-12 fusion proteins and conjugates; methods of manufacturing; pharmaceutical compositions; and methods of use including e.g., methods of treating diseases (e.g., cancer).

In one aspect, provided herein are human interleukin 12 p40 (hIL-12p40) polypeptides comprising an amino acid sequence (a) at least 85%, 86%, 87%, 88%, 89%, 90%, 91%, 92%, 93%, 94%, 95%, 96%, 97%, 98%, or 99% identical to the amino acid sequence of SEQ ID NO: 33; and (b) comprising or consisting of an amino acid substitution at each of amino acid positions (i) W37, F82, and K219; (ii) W37, F82, and K217; (iii) K106, K217, and K219; (iv) W37 and F82; (v) W37 and K217; (vi) W37 and K219; (vii) W37 and K106; (viii) F82 and K106; (xiv) F82 and K217; (xv) F82 and K219; (xvi) K217 and K219; (xvii) K106 and K217; or (xviii) K106 and K219, amino acid numbering relative to the amino acid sequence of SEQ ID NO: 32.

In some embodiments, the hIL-12p40 polypeptide comprises or consists of each of the following amino acid substitutions (i) W37A, F82A, and K219A; (ii) W37A, F82A, and K217A; (iii) K106A, K217A, and K219A; (iv) W37A and F82A; (v) W37A and K217A; (vi) W37A and K219A; (vii) W37A and K106A; (viii) F82A and K106A; (xiv) F82A and K217A; (xv) F82A and K219A; (xvi) K217A and K219A; (xvii) K106A and K217A; or (xviii) K106A and K219A, amino acid numbering relative to the amino acid sequence of SEQ ID NO: 32.

In some embodiments, the hIL-12p40 polypeptide comprises or consists of an amino acid substitution at each of amino acid positions W37, F82, and K219, amino acid numbering relative to the amino acid sequence of SEQ ID NO: 32.

In some embodiments, the hIL-12p40 polypeptide comprises or consists of each of the following amino acid substitutions W37A, F82A, and K219A, amino acid numbering relative to the amino acid sequence of SEQ ID NO: 32.

In some embodiments, the amino acid sequence of the hIL-12p40 polypeptide comprises or consists of a set of amino acid substitutions set forth in the amino acid sequence of any one polypeptide set forth in Table 5 (amino acid substitutions relative to the amino acid sequence of SEQ ID NO: 33); and other than the set of amino acid substitutions, the amino acid sequence of the hIL-12p40 polypeptide is at least 85%, 86%, 87%, 88%, 89%, 90%, 91%, 92%, 93%, 94%, 95%, 96%, 97%, 98%, 99%, or 100% identical to the amino acid sequence of the polypeptide set forth in Table 5.

In some embodiments, the amino acid sequence of the hIL-12p40 polypeptide comprises or consists of a set of amino acid substitutions set forth in the amino acid sequence of any one SEQ ID NOS: 38-65 (amino acid substitutions relative to the amino acid sequence of SEQ ID NO: 33); and other than the set of amino acid substitutions, the amino acid sequence of the hIL-12p40 polypeptide is at least 85%, 86%, 87%, 88%, 89%, 90%, 91%, 92%, 93%, 94%, 95%, 96%, 97%, 98%, 99%, or 100% identical to the amino acid sequence of set forth in the any one of SEQ ID NOS: 38-65.

In some embodiments, the amino acid sequence of the hIL-12p40 polypeptide comprises or consists of a set of amino acid substitutions set forth in the amino acid sequence of any one SEQ ID NOS: 38-51 (amino acid substitutions relative to the amino acid sequence of SEQ ID NO: 33); and other than the set of amino acid substitutions, the amino acid sequence of the hIL-12p40 polypeptide is at least 85%, 86%, 87%, 88%, 89%, 90%, 91%, 92%, 93%, 94%, 95%, 96%, 97%, 98%, 99%, or 100% identical to the amino acid sequence of set forth in the any one of SEQ ID NOS: 38-51.

In some embodiments, the amino acid sequence of the hIL-12p40 polypeptide comprises or consists of a set of amino acid substitutions set forth in the amino acid sequence of any one SEQ ID NOS: 52-65 (amino acid substitutions relative to the amino acid sequence of SEQ ID NO: 33); and other than the set of amino acid substitutions, the amino acid sequence of the hIL-12p40 polypeptide is at least 85%, 86%, 87%, 88%, 89%, 90%, 91%, 92%, 93%, 94%, 95%, 96%, 97%, 98%, 99%, or 100% identical to the amino acid sequence of set forth in the any one of SEQ ID NOS: 52-65.

In some embodiments, the amino acid sequence of the hIL-12p40 polypeptide comprises or consists of a set of amino acid substitutions set forth in the amino acid sequence SEQ ID NO: 38 or 52 (amino acid substitutions relative to the amino acid sequence of SEQ ID NO: 33); and other than the set of amino acid substitutions, the amino acid sequence of the hIL-12p40 polypeptide is at least 85%, 86%, 87%, 88%, 89%, 90%, 91%, 92%, 93%, 94%, 95%, 96%, 97%, 98%, 99%, or 100% identical to the amino acid sequence of set forth in SEQ ID NOS: 38 or 52.

In some embodiments, the amino acid sequence of the hIL-12p40 polypeptide comprises or consists of a set of amino acid substitutions set forth in the amino acid sequence SEQ ID NO: 38 (amino acid substitutions relative to the amino acid sequence of SEQ ID NO: 33); and other than the set of amino acid substitutions, the amino acid sequence of the hIL-12p40 polypeptide is at least 85%, 86%, 87%, 88%, 89%, 90%, 91%, 92%, 93%, 94%, 95%, 96%, 97%, 98%, 99%, or 100% identical to the amino acid sequence of set forth in SEQ ID NOS: 38.

In some embodiments, the amino acid sequence of the hIL-12p40 polypeptide is 100% identical to the amino acid sequence of SEQ ID NO: 38.

In some embodiments, the hIL-12p40 polypeptide specifically binds the hIL-12 receptor (hIL-12R).

In some embodiments, when combined with a hIL-12p35 protein the hIL-12p40 protein mediates a lower increase in the level of phosphorylated STAT4 (pSTAT4) in cells expressing the hIL-12R on the surface relative to the increase in pSTAT4 mediated by a suitable control (e.g., a reference hIL-12p40 protein (e.g., SEQ ID NO: 33)).

In some embodiments, when combined with a hIL-12p35 protein the hIL-12p40 protein mediates from about a 0.5-1000 fold, 0.5-100 fold, 0.5-10 fold, 0.5-5 fold, 0.5-2 fold, 1-1000 fold, 1-100 fold, 1-10 fold, 1-5 fold, 1-2 fold, 10-1000 fold, or 100-1000 fold lower increase in the level of phosphorylated STAT4 (pSTAT4) in cells expressing the hIL-12R on the surface relative to the increase in pSTAT4 mediated by a suitable control (e.g., a reference hIL-12p40 protein (e.g., SEQ ID NO: 33)).

In some embodiments, when combined with a hIL-12p35 protein the hIL-12p40 protein mediates a lower increase the level of interferon gamma (IFN-γ) produced by expressing the hIL-12R on the surface relative to the increase in the level of IFN-γ produced in the presence of a suitable control (e.g., a reference hIL-12p40 protein (e.g., SEQ ID NO: 33)).

In some embodiments, when combined with a hIL-12p35 protein the hIL-12p40 protein mediates from about a 0.5-1000 fold, 0.5-100 fold, 0.5-10 fold, 0.5-5 fold, 0.5-2 fold, 1-1000 fold, 1-100 fold, 1-10 fold, 1-5 fold, 1-2 fold, 10-1000 fold, or 100-1000 fold lower increase in the level of IFN-γ produced by cells expressing the hIL-12R on the surface, relative to the increase in the level of IFN-γ produced in the presence of a suitable control (e.g., a reference hIL-12p40 protein (e.g., SEQ ID NO: 33)).

In one aspect, provided herein are human interleukin 12p35 (hIL-12p35) polypeptides comprising an amino acid sequence (a) at least 85%, 86%, 87%, 88%, 89%, 90%, 91%, 92%, 93%, 94%, 95%, 96%, 97%, 98%, or 99% identical to the amino acid sequence of SEQ ID NO: 31; and (b) comprises or consists of an amino acid modification (e.g., substitution, addition, deletion (e.g., substitution)) at one or more of the following amino acid positions E60, F61, P63, K150, F188, Y189A, amino acid numbering relative to the amino acid sequence of SEQ ID NO: 30.

In some embodiments, the hIL-12p35 polypeptide comprises or consists of an amino acid modification (e.g., substitution, addition, deletion (e.g., substitution)) at each of the following amino acid positions (i) F188; (ii) Y189; (iii) F188 and Y189; or (iv) E60, F61, P63, K150, and F188, amino acid numbering relative to the amino acid sequence of SEQ ID NO: 30.

In some embodiments, the hIL-12p35 polypeptide comprises or consists of one or more of the following amino acid substitutions: E60K, F61H, P63S, K150H, F188P, F188A, and/or Y189A, amino acid numbering relative to the amino acid sequence of SEQ ID NO: 30.

In some embodiments, the amino acid sequence of the hIL-12p35 polypeptide comprises or consists of a set of amino acid substitutions set forth in the amino acid sequence of any one SEQ ID NOS: 111-114 (relative to the amino acid sequence of SEQ ID NO: 31); and other than the set of amino acid substitutions, the amino acid sequence of the hIL-12p35 polypeptide is at least 85%, 86%, 87%, 88%, 89%, 90%, 91%, 92%, 93%, 94%, 95%, 96%, 97%, 98%, 99%, or 100% identical to the amino acid sequence of set forth in the any one of SEQ ID NOS: 111-114.

In some embodiments, the hIL-12p35 polypeptide comprises or consists of each of the following amino acid substitutions: (i) F188A; (ii) Y189A; (iii) F188A and Y189A; or (iv) E60K, F61H, P63S, K150H, and F188P, amino acid numbering relative to the amino acid sequence of SEQ ID NO: 30.

In some embodiments, the amino acid sequence of the hIL-12p35 polypeptide comprises or consists of a deletion of amino acids A55-K92, N50-K92, M51-K92, L52-K92, Q53-K92, K54-K92, N50-N93, M51-N93, L52-N93, Q53-N93, K54-N93, N50-E94, M51-E94, L52-E94, Q53-E94, K54-E94, N50-S95, M51-S95, L52-S95, Q53-S95, K54-S95, N50-C96, M51-C96, L52-C96, Q53-C96, K54-C96, N50-L97, M51-L97, L52-L97, Q53-L97, K54-L97, N50-P87, M51-P87, L52-P87, Q53-P87, K54-P87, N50-L88, M51-L88, L52-L88, Q53-L88, K54-L88, N50-E89, M51-E89, L52-E89, Q53-E89, K54-E89, N50-L90, M51-L90, L52-L90, Q53-L90, K54-L90, N50-T91, M51-T91, L52-T91, Q53-T91, K54-T91, R56-K92, Q57-K92, T58-K92, L59-K92, E60-K92 A55-N93, R56-N93, Q57-N93, T58-N93, L59-N93, E60-N93, A55-E94, R56-E94, Q57-E94, T58-E94, L59-E94, E60-E94, A55-S95, R56-S95, Q57-S95, T58-S95, L59-S95, E60-S95, A55-C96, R56-C96, Q57-C96, T58-C96, L59-C96, E60-C96, A55-L97, R56-L97, Q57-L97, T58-L97, L59-L97, E60-L97, A55-P87, R56-P87, Q57-P87, T58-P87, L59-P87, E60-P87, A55-L88, R56-L88, Q57-L88, T58-L88, L59-L88, E60-L88, A55-E89, R56-E89, Q57-E89, T58-E89, L59-E89, E60-E89, A55-L90, R56-L90, Q57-L90, T58-L90, L59-L90, E60-L90, A55-T91, R56-T91, Q57-T91, T58-T91, L59-T91, or E60-T91 (amino acid numbering relative to the amino acid sequence of SEQ ID NO: 30), and other than the deletion of amino acids A55-K92, N50-K92, M51-K92, L52-K92, Q53-K92, K54-K92, N50-N93, M51-N93, L52-N93, Q53-N93, K54-N93, N50-E94, M51-E94, L52-E94, Q53-E94, K54-E94, N50-S95, M51-S95, L52-S95, Q53-S95, K54-S95, N50-C96, M51-C96, L52-C96, Q53-C96, K54-C96, N50-L97, M51-L97, L52-L97, Q53-L97, K54-L97, N50-P87, M51-P87, L52-P87, Q53-P87, K54-P87, N50-L88, M51-L88, L52-L88, Q53-L88, K54-L88, N50-E89, M51-E89, L52-E89, Q53-E89, K54-E89, N50-L90, M51-L90, L52-L90, Q53-L90, K54-L90, N50-T91, M51-T91, L52-T91, Q53-T91, K54-T91, R56-K92, Q57-K92, T58-K92, L59-K92, E60-K92 A55-N93, R56-N93, Q57-N93, T58-N93, L59-N93, E60-N93, A55-E94, R56-E94, Q57-E94, T58-E94, L59-E94, E60-E94, A55-S95, R56-S95, Q57-S95, T58-S95, L59-S95, E60-S95, A55-C96, R56-C96, Q57-C96, T58-C96, L59-C96, E60-C96, A55-L97, R56-L97, Q57-L97, T58-L97, L59-L97, E60-L97, A55-P87, R56-P87, Q57-P87, T58-P87, L59-P87, E60-P87, A55-L88, R56-L88, Q57-L88, T58-L88, L59-L88, E60-L88, A55-E89, R56-E89, Q57-E89, T58-E89, L59-E89, E60-E89, A55-L90, R56-L90, Q57-L90, T58-L90, L59-L90, E60-L90, A55-T91, R56-T91, Q57-T91, T58-T91, L59-T91, or E60-T91 the amino acid sequence of the polypeptide is at least 85%, 86%, 87%, 88%, 89%, 90%, 91%, 92%, 93%, 94%, 95%, 96%, 97%, 98%, or 99% identical to the amino acid sequence of SEQ ID NO: 31.

In some embodiments, the amino acid sequence of the hIL-12p35 polypeptide comprises or consists of a deletion of amino acids A55-K92 (amino acid numbering relative to the amino acid sequence of SEQ ID NO: 30), and other than the deletion of amino acids A55-K92 the amino acid sequence of the polypeptide is at least 85%, 86%, 87%, 88%, 89%, 90%, 91%, 92%, 93%, 94%, 95%, 96%, 97%, 98%, or 99% identical to the amino acid sequence of SEQ ID NO: 31.

In some embodiments, the amino acid sequence of the hIL-12p35 polypeptide comprises or consists of a set of amino acid deletions set forth in the amino acid sequence of any one SEQ ID NOS: 110 (relative to the amino acid sequence of SEQ ID NO: 31); and other than the set of amino acid deletions, the amino acid sequence of the hIL-12p35 polypeptide is at least 85%, 86%, 87%, 88%, 89%, 90%, 91%, 92%, 93%, 94%, 95%, 96%, 97%, 98%, 99%, or 100% identical to the amino acid sequence of set forth in the any one of SEQ ID NOS: 110.

In one aspect, provided herein are single chain hIL-12 (schIL-12) polypeptides comprising a hIL-12p40 polypeptide described herein operably connected to a hIL-12p35 polypeptide. In some embodiments, the amino acid sequence of the hIL-12p35 polypeptide is at least 85%, 86%, 87%, 88%, 89%, 90%, 91%, 92%, 93%, 94%, 95%, 96%, 97%, 98%, 99%, or 100% identical to the amino acid sequence of a polypeptide set forth in Table 6. In some embodiments, the amino acid sequence of the hIL-12p35 polypeptide is at least 85%, 86%, 87%, 88%, 89%, 90%, 91%, 92%, 93%, 94%, 95%, 96%, 97%, 98%, 99%, or 100% identical to the amino acid sequence of any one of SEQ ID NOS: 31 or 110-114. In some embodiments, the hIL-12p35 polypeptide is a hIL-12p35 polypeptide described herein. In some embodiments, the hIL-12p40 polypeptide is operably connected to the hIL-12p35 polypeptide via a peptide linker. In some embodiments, the polypeptide comprises from N- to C-terminus: the hIL-12p40 polypeptide, a peptide linker, and the hIL-12p35 polypeptide. In some embodiments, the polypeptide comprises from N- to C-terminus: the hIL-12p35 polypeptide, a peptide linker, and the hIL-12p40 polypeptide.

In one aspect, provided herein are schIL-12 polypeptides comprising the hIL-12p35 polypeptide described herein operably connected to a hIL-12p40 polypeptide. In some embodiments, the amino acid sequence of the hIL-12p40 polypeptide is at least 85%, 86%, 87%, 88%, 89%, 90%, 91%, 92%, 93%, 94%, 95%, 96%, 97%, 98%, 99%, or 100% identical to the amino acid sequence of a polypeptide set forth in Table 10. In some embodiments, the amino acid sequence of the hIL-12p40 polypeptide is at least 85%, 86%, 87%, 88%, 89%, 90%, 91%, 92%, 93%, 94%, 95%, 96%, 97%, 98%, 99%, or 100% identical to the amino acid sequence of any one of SEQ ID NOS: 38-51 or 90-109. In some embodiments, the hIL-12p40 polypeptide is a hIL-12p40 polypeptide described herein. In some embodiments, the hIL-12p40 polypeptide is operably connected to the hIL-12p35 polypeptide via a peptide linker. In some embodiments, the polypeptide comprises from N- to C-terminus: the hIL-12p40 polypeptide, a peptide linker, and the hIL-12p35 polypeptide. In some embodiments, the polypeptide comprises from N- to C-terminus: the hIL-12p35 polypeptide, a peptide linker, and the hIL-12p40 polypeptide.

In one aspect, provided herein are fusion proteins comprising a hIL-12p40 polypeptide described herein (e.g., a variant hIL-12p40 polypeptide), a hIL-12p35 polypeptide; and a heterologous moiety.

In some embodiments, the amino acid sequence of the hIL-12p35 polypeptide is at least 85%, 86%, 87%, 88%, 89%, 90%, 91%, 92%, 93%, 94%, 95%, 96%, 97%, 98%, 99%, or 100% identical to the amino acid sequence of a polypeptide set forth in Table 6.

In some embodiments, the amino acid sequence of the hIL-12p35 polypeptide is at least 85%, 86%, 87%, 88%, 89%, 90%, 91%, 92%, 93%, 94%, 95%, 96%, 97%, 98%, 99%, or 100% identical to the amino acid sequence of any one of SEQ ID NOS: 31 or 110-114. In some embodiments, the amino acid sequence of the hIL-12p35 polypeptide is at least 85%, 86%, 87%, 88%, 89%, 90%, 91%, 92%, 93%, 94%, 95%, 96%, 97%, 98%, 99%, or 100% identical to the amino acid sequence of SEQ ID NO: 31. In some embodiments, the amino acid sequence of the hIL-12p40 polypeptide is at least 85%, 86%, 87%, 88%, 89%, 90%, 91%, 92%, 93%, 94%, 95%, 96%, 97%, 98%, 99%, or 100% identical to the amino acid sequence of a polypeptide set forth in Table 10. In some embodiments, the amino acid sequence of the hIL-12p35 polypeptide is at least 85%, 86%, 87%, 88%, 89%, 90%, 91%, 92%, 93%, 94%, 95%, 96%, 97%, 98%, 99%, or 100% identical to the amino acid sequence of any one of SEQ ID NOS: 33 or 38-51 or 90-109. In some embodiments, the amino acid sequence of the hIL-12p35 polypeptide is at least 85%, 86%, 87%, 88%, 89%, 90%, 91%, 92%, 93%, 94%, 95%, 96%, 97%, 98%, 99%, or 100% identical to the amino acid sequence of SEQ ID NO: 33 or 38.

In some embodiments, the hIL-12p40 polypeptide and the hIL-12p35 polypeptide arc operably connected as a schIL-12 polypeptide. In some embodiments, the hIL-12p40 polypeptide is operably connected to the hIL-12p35 polypeptide via a peptide linker. In some embodiments, the polypeptide comprises from N- to C-terminus: the hIL-12p40 polypeptide, a peptide linker, and the hIL-12p35 polypeptide. In some embodiments, the polypeptide comprises from N- to C-terminus: the hIL-12p35 polypeptide, a peptide linker, and the hIL-12p40 polypeptide.

In some embodiments, the fusion protein mediates a lower increase in the level of phosphorylated STAT4 (pSTAT4) in cells expressing the hIL-12R on the surface relative to the increase in pSTAT4 mediated by a suitable control (e.g., a reference hIL-12 fusion protein (e.g., SEQ ID NOS: 371, 372, 383)). In some embodiments, the fusion protein mediates from about a 0.5-1000-fold, 0.5-100-fold, 0.5-10-fold, 0.5-5 fold, 0.5-2 fold, 1-1000 fold, 1-100 fold, 1-10 fold, 1-5 fold, 1-2 fold, 10-1000 fold, or 100-1000 fold lower increase in the level of phosphorylated STAT4 (pSTAT4) in cells expressing the hIL-12R on the surface relative to the increase in pSTAT4 mediated by a suitable control (e.g., a reference hIL-12 fusion protein (e.g., SEQ ID NOS: 371, 372, 383)). In some embodiments, the fusion protein mediates a lower increase the level of interferon gamma (IFN-γ) produced by expressing the hIL-12R on the surface relative to the increase in the level of IFN-γ produced in the presence of a suitable control (e.g., a reference hIL-12 fusion protein (e.g., SEQ ID NOS: 371, 372, 383)). In some embodiments, the fusion protein mediates from about a 0.5-1000 fold, 0.5-100 fold, 0.5-10 fold, 0.5-5 fold, 0.5-2 fold, 1-1000 fold, 1-100 fold, 1-10 fold, 1-5 fold, 1-2 fold, 10-1000 fold, or 100-1000 fold lower increase in the level of IFN-γ produced by expressing the hIL-12R on the surface, relative to the increase in the level of IFN-γ produced in the presence of a suitable control (e.g., a reference hIL-12 fusion protein (e.g., SEQ ID NOS: 371, 372, 383)).

In some embodiments, the heterologous moiety comprises or consist of an antibody (or antigen binding domain thereof) and/or one or more Fc region. In some embodiments, the heterologous moiety comprises or consist of an antibody (or antigen binding domain thereof). In some embodiments, the heterologous moiety comprises or consists of a full-length antibody, scFv, (scFv) 2, scFv-Fc, Fab, Fab′, F(ab′) 2, Fab-Fc, a single domain antibody (e.g., VHH), or single domains antibody-Fc (e.g., VHH-Fc. In some embodiments, the antibody (or antigen binding domain thereof) comprises a first variable heavy chain region (VH) that comprises three VH complementarity determining regions (VH CDRs): VH CDR1, VH CDR2, and VH CDR3; and a first variable light chain region (VL) that comprises three VL CDRs: VL CDR1, VL CDR2, and VL CDR3. In some embodiments, the heterologous moiety comprises or consists of a full-length antibody.

In some embodiments, the antibody (or antigen binding domain thereof) specifically binds to a human tumor associated antigen (hTAA).

In some embodiments, the antibody (or antigen binding domain thereof) specifically binds human carbonic anhydrase IX (hCAIX).

In some embodiments, the amino acid sequence of the VH CDR1, VH CDR2, VH CDR3, VL CDR1, VL CDR2, and VL CDR3 each comprises or consists of the amino acid sequence of a VH CDR1, VH CDR2, VH CDR3, VL CDR1, VL CDR2, and VL CDR3 of an antibody set forth in Table 17.

In some embodiments, the amino acid sequence of VH CDR1 comprises the amino acid sequence of SEQ ID NO: 237, or the amino acid sequence of SEQ ID NO: 237 with 1, 2, or 3 amino acid modifications (e.g., substitution, deletion, addition, etc.); the amino acid sequence of VH CDR2 comprises the amino acid sequence of SEQ ID NO: 238, or the amino acid sequence of SEQ ID NO: 238 with 1, 2, or 3 amino acid modifications (e.g., substitution, deletion, addition, etc.); the amino acid sequence of VH CDR3 comprises the amino acid sequence of SEQ ID NO: 239, or the amino acid sequence of SEQ ID NO: 239 with 1, 2, or 3 amino acid modifications (e.g., substitution, deletion, addition, etc.); the amino acid sequence of VL CDR1 comprises the amino acid sequence of SEQ ID NO: 240, or the amino acid sequence of SEQ ID NO: 240 with 1, 2, or 3 amino acid modifications (e.g., substitution, deletion, addition, etc.); the amino acid sequence of VL CDR2 comprises the amino acid sequence of SEQ ID NO: 241 or the amino acid sequence of SEQ ID NO: 242 with 1, 2, or 3 amino acid modifications (e.g., substitution, deletion, addition, etc.); and the amino acid sequence of VL CDR3 comprises the amino acid sequence of SEQ ID NO: 243, or the amino acid sequence of SEQ ID NO: 243 with 1, 2, or 3 amino acid modifications (e.g., substitution, deletion, addition, etc.).

In some embodiments, the amino acid sequence of the VH is at least 85%, 86%, 87%, 88%, 89%, 90%, 91%, 92%, 93%, 94%, 95%, 96%, 97%, 98%, 99%, or 100% identical to the amino acid sequence of any VH polypeptide set forth in Table 17; and the amino acid sequence of the VL comprises or consists of an amino acid sequence at least 85%, 86%, 87%, 88%, 89%, 90%, 91%, 92%, 93%, 94%, 95%, 96%, 97%, 98%, 99%, or 100% identical to the amino acid sequence of any VL polypeptide set forth in Table 17.

In some embodiments, the amino acid sequence of the VH comprises or consists of an amino acid sequence at least 85%, 86%, 87%, 88%, 89%, 90%, 91%, 92%, 93%, 94%, 95%, 96%, 97%, 98%, 99%, or 100% identical to the amino acid sequence of any one of SEQ ID NOS: 7, 246, 256, 264, 274, or 284; and the amino acid sequence of the VL comprises or consists of an amino acid sequence at least 85%, 86%, 87%, 88%, 89%, 90%, 91%, 92%, 93%, 94%, 95%, 96%, 97%, 98%, 99%, or 100% identical to the amino acid sequence of any one of SEQ ID NOS: 12, 247, 257, 265, 275, or 285.

In some embodiments, the amino acid sequence of the VH comprises or consists of an amino acid sequence at least 85%, 86%, 87%, 88%, 89%, 90%, 91%, 92%, 93%, 94%, 95%, 96%, 97%, 98%, 99%, or 100% identical to the amino acid sequence of SEQ ID NO: 7; and the amino acid sequence of the VL comprises or consists of an amino acid sequence at least 85%, 86%, 87%, 88%, 89%, 90%, 91%, 92%, 93%, 94%, 95%, 96%, 97%, 98%, 99%, or 100% identical to the amino acid sequence of SEQ ID NO: 12.

In some embodiments, the amino acid sequence of the VH comprises or consists of an amino acid sequence at least 85%, 86%, 87%, 88%, 89%, 90%, 91%, 92%, 93%, 94%, 95%, 96%, 97%, 98%, 99%, or 100% identical to the amino acid sequence of SEQ ID NO: 7; and the amino acid sequence of the VL comprises or consists of an amino acid sequence at least 85%, 86%, 87%, 88%, 89%, 90%, 91%, 92%, 93%, 94%, 95%, 96%, 97%, 98%, 99%, or 100% identical to the amino acid sequence of SEQ ID NO: 15.

In some embodiments, the fusion protein comprises a first Fc region comprising a CH2 region and a CH3 region; and the second Fc region comprising a CH2 region and a CH3 region. In some embodiments, (a) the first Fc region comprises a CH2 region and a CH3 region; and the second Fc region comprises a CH2 region and a CH3 region; or (b) the first Fc region comprises a hinge region, a CH2 region, and a CH3 region; and the second Fc region comprises a hinge region, a CH2 region, and a CH3 region. In some embodiments, the first Fc region and the second Fc region are each a hIgG1 or hIgG4 Fc region, or functional variant thereof. In some embodiments, the first Fc region and the second Fc region are part of a full-length antibody.

In some embodiments, the CH3 region of the first Fc region and the CH3 region of the second Fc region each comprise at least one amino acid modification that promotes heterodimerization of the first Fc region and the second Fc region.

In some embodiments, the first Fc region comprises an amino acid substitution at amino acid position T366, L368, and Y407, numbering according to EU index of Kabat. In some embodiments, the first Fc region comprises the following amino acid substitutions T366S, L368A, and Y407V, numbering according to EU index of Kabat. In some embodiments, the first Fc region comprises an amino acid substitution at amino acid position Y349, numbering according to EU index of Kabat. In some embodiments, the first Fc region comprises the following amino acid substitution Y349C, numbering according to EU index of Kabat. In some embodiments, the first Fc region comprises an amino acid substitution at amino acid position T366, L368, Y407, and Y349, numbering according to EU index of Kabat. In some embodiments, the first Fc region comprises the following amino acid substitutions T366S, L368A, Y407V, and Y349C, numbering according to EU index of Kabat. In some embodiments, the second Fc region comprises an amino acid substitution at amino acid position T366, numbering according to EU index of Kabat. In some embodiments, the second Fc region comprises the following amino acid substitution T366W, numbering according to EU index of Kabat. In some embodiments, the second Fc region comprises an amino acid substitution at amino acid position S354, numbering according to EU index of Kabat. In some embodiments, the second Fc region comprises the following amino acid substitution S354C, numbering according to EU index of Kabat. In some embodiments, the second Fc region comprises an amino acid substitution at amino acid position T366 and S354, numbering according to EU index of Kabat. In some embodiments, the second Fc region comprises the following amino acid substitutions T366W and S354C, numbering according to EU index of Kabat. In some embodiments, the first Fc region comprises the following amino acid substitutions T366S, L368A, Y407V, and Y349C, and wherein the second Fc region comprises the following amino acid substitutions T366W and S354C, numbering according to EU index of Kabat.

In some embodiments, the second Fc region comprises an amino acid substitution at amino acid position T366, L368, and Y407, numbering according to EU index of Kabat. In some embodiments, the second Fc region comprises the following amino acid substitutions T366S, L368A, and Y407V, numbering according to EU index of Kabat. In some embodiments, the second Fc region comprises an amino acid substitution at amino acid position Y349, numbering according to EU index of Kabat. In some embodiments, the second Fc region comprises the following amino acid substitution Y349C, numbering according to EU index of Kabat. In some embodiments, the second Fc region comprises an amino acid substitution at amino acid position T366, L368, Y407, and Y349, numbering according to EU index of Kabat. In some embodiments, the second Fc region comprises the following amino acid substitutions T366S, L368A, Y407V, and Y349C, numbering according to EU index of Kabat. In some embodiments, the first Fc region comprises an amino acid substitution at amino acid position T366, numbering according to EU index of Kabat. In some embodiments, the first Fc region comprises the following amino acid substitution T366W, numbering according to EU index of Kabat. In some embodiments, the first Fc region comprises an amino acid substitution at amino acid position S354, numbering according to EU index of Kabat. In some embodiments, the first Fc region comprises the following amino acid substitution S354C, numbering according to EU index of Kabat. In some embodiments, the first Fc region comprises an amino acid substitution at amino acid position T366 and S354, numbering according to EU index of Kabat. In some embodiments, the first Fc region comprises the following amino acid substitutions T366W and S354C, numbering according to EU index of Kabat. In some embodiments, the first Fc region comprises the following amino acid substitutions T366S, L368A, Y407V, and Y349C, and wherein the first Fc region comprises the following amino acid substitutions T366W and S354C, numbering according to EU index of Kabat.

In some embodiments, the first Fc region and the second Fc region each comprises at least one amino acid modification (e.g., substitution, deletion, addition) that reduces or eliminates an Fc region effector function compared to a reference Fc region that does not contain the at least one amino acid modification (e.g., substitution, deletion, addition). In some embodiments, the at least one effector function comprises the ability of the Fc region to induce antibody-dependent cellular cytotoxicity (ADCC), antibody-dependent cellular phagocytosis (ADCP), or complement dependent cytotoxicity (CDC), bind an Fc receptor (e.g., an Fcγ receptor), or any combination thereof.

In some embodiments, the first Fc region and the second Fc region each comprises an amino acid substitution at one, two, or three of amino acid positions L234, L235, and/or P329, numbering according to EU index of Kabat. In some embodiments, the first Fc region and the second Fc region each comprises one, two, or three of the following amino acid substitutions: L234A, L235A, and/or P329G or P329A, numbering according to EU index of Kabat. In some embodiments, the first Fc region and the second Fc region each comprise a L234A and L235A amino acid substitution, numbering according to EU index of Kabat. In some embodiments, the first Fc region and the second Fc region each comprise a L234A, L235A, and P329A amino acid substitution, numbering according to EU index of Kabat. In some embodiments, the first Fc region and the second Fc region each comprise a L234A, L235A, and P329G amino acid substitution, numbering according to EU index of Kabat.

In some embodiments, the N-terminus of the hIL-12p40 polypeptide is operably connected to the C-terminus of the first Fc region; and wherein the N-terminus of the hIL-12p35 polypeptide is operably connected to the C-terminus of the second Fc region. In some embodiments, the hIL-12p40 polypeptide is operably connected to the first Fc region via a first peptide linker; and the hIL-12p40 polypeptide is operably connected to the second Fc region via a second peptide linker. In some embodiments, the amino acid sequence of the first peptide linker comprises or consists of the amino acid sequence of a peptide linker set forth in Table 18; and wherein the amino acid sequence of the second peptide linker comprises or consists of the amino acid sequence of a peptide linker set forth in Table 18. In some embodiments, the amino acid sequence of the first peptide linker comprises or consists of the amino acid sequence of any one of SEQ ID NOS: 66-81, 88-303, or 369; and wherein the amino acid sequence of the second peptide linker comprises or consists of the amino acid sequence of any one of SEQ ID NOS: 66-81, 288-303, or 369. In some embodiments, the amino acid sequence of the first peptide linker comprises or consists of the amino acid sequence of SEQ ID NO: 72; and wherein the amino acid sequence of the second peptide linker comprises or consists of the amino acid sequence of SEQ ID NO: 72.

In some embodiments, the N-terminus of the hIL-12p35 polypeptide is operably connected to the C-terminus of the first Fc region; and wherein the N-terminus of the hIL-12p40 polypeptide is operably connected to the C-terminus of the second Fc region. In some embodiments, the hIL-12p40 polypeptide is operably connected to the second Fc region via a first peptide linker; and the hIL-12p40 polypeptide is operably connected to the first Fc region via a second peptide linker. In some embodiments, the amino acid sequence of the first peptide linker comprises or consists of the amino acid sequence of a peptide linker set forth in Table 18; and wherein the amino acid sequence of the second peptide linker comprises or consists of the amino acid sequence of a peptide linker set forth in Table 18. In some embodiments, the amino acid sequence of the first peptide linker comprises or consists of the amino acid sequence of any one of SEQ ID NOS: 66-81, 288-303, or 369; and wherein the amino acid sequence of the second peptide linker comprises or consists of the amino acid sequence of any one of SEQ ID NOS: 66-81, 288-303, or 369. In some embodiments, the amino acid sequence of the first peptide linker comprises or consists of the amino acid sequence of SEQ ID NO: 72; and wherein the amino acid sequence of the second peptide linker comprises or consists of the amino acid sequence of SEQ ID NO: 72.

In some embodiments, the hIL-12p40 polypeptide and the hIL-12p35 polypeptide are operably connected as a schIL-12 polypeptide, and wherein the N-terminus of the schIL-12 polypeptide is operably connected to the C-terminus of the first Fc region or the C-terminus of the second Fc region. In some embodiments, the schIL-12 polypeptide is operably connected to the first Fc region or the second Fc region via a first peptide linker. In some embodiments, the amino acid sequence of the first peptide linker comprises or consists of the amino acid sequence of a peptide linker set forth in Table 18. In some embodiments, the amino acid sequence of the first peptide linker comprises or consists of the amino acid sequence of any one of SEQ ID NOS: 66-81, 288-303, or 369. In some embodiments, the amino acid sequence of the first peptide comprises or consists of the amino acid sequence of SEQ ID NO: 72.

In one aspect, provided herein are fusion proteins comprising a hIL-12p40 polypeptide; a hIL-12p35 polypeptide described herein (e.g., a variant hIL-12p35 polypeptide); and a heterologous moiety.

In some embodiments, the amino acid sequence of the hIL-12p35 polypeptide is at least 85%, 86%, 87%, 88%, 89%, 90%, 91%, 92%, 93%, 94%, 95%, 96%, 97%, 98%, 99%, or 100% identical to the amino acid sequence of a polypeptide set forth in Table 6.

In some embodiments, the amino acid sequence of the hIL-12p35 polypeptide is at least 85%, 86%, 87%, 88%, 89%, 90%, 91%, 92%, 93%, 94%, 95%, 96%, 97%, 98%, 99%, or 100% identical to the amino acid sequence of any one of SEQ ID NOS: 31 or 110-114. In some embodiments, the amino acid sequence of the hIL-12p35 polypeptide is at least 85%, 86%, 87%, 88%, 89%, 90%, 91%, 92%, 93%, 94%, 95%, 96%, 97%, 98%, 99%, or 100% identical to the amino acid sequence of SEQ ID NO: 31. In some embodiments, the amino acid sequence of the hIL-12p40 polypeptide is at least 85%, 86%, 87%, 88%, 89%, 90%, 91%, 92%, 93%, 94%, 95%, 96%, 97%, 98%, 99%, or 100% identical to the amino acid sequence of a polypeptide set forth in Table 10. In some embodiments, the amino acid sequence of the hIL-12p35 polypeptide is at least 85%, 86%, 87%, 88%, 89%, 90%, 91%, 92%, 93%, 94%, 95%, 96%, 97%, 98%, 99%, or 100% identical to the amino acid sequence of any one of SEQ ID NOS: 33 or 38-51 or 90-109. In some embodiments, the amino acid sequence of the hIL-12p35 polypeptide is at least 85%, 86%, 87%, 88%, 89%, 90%, 91%, 92%, 93%, 94%, 95%, 96%, 97%, 98%, 99%, or 100% identical to the amino acid sequence of SEQ ID NO: 33 or 38.

In some embodiments, the hIL-12p40 polypeptide and the hIL-12p35 polypeptide are operably connected as a schIL-12 polypeptide. In some embodiments, the hIL-12p40 polypeptide is operably connected to the hIL-12p35 polypeptide via a peptide linker. In some embodiments, the polypeptide comprises from N- to C-terminus: the hIL-12p40 polypeptide, a peptide linker, and the hIL-12p35 polypeptide. In some embodiments, the polypeptide comprises from N- to C-terminus: the hIL-12p35 polypeptide, a peptide linker, and the hIL-12p40 polypeptide.

In some embodiments, the fusion protein mediates a lower increase in the level of phosphorylated STAT4 (pSTAT4) in cells expressing the hIL-12R on the surface relative to the increase in pSTAT4 mediated by a suitable control (e.g., a reference hIL-12 fusion protein (e.g., SEQ ID NOS: 371, 372, 383)). In some embodiments, the fusion protein mediates from about a 0.5-1000-fold, 0.5-100-fold, 0.5-10-fold, 0.5-5 fold, 0.5-2 fold, 1-1000 fold, 1-100 fold, 1-10 fold, 1-5 fold, 1-2 fold, 10-1000 fold, or 100-1000 fold lower increase in the level of phosphorylated STAT4 (pSTAT4) in cells expressing the hIL-12R on the surface relative to the increase in pSTAT4 mediated by a suitable control (e.g., a reference hIL-12 fusion protein (e.g., SEQ ID NOS: 371, 372, 383)). In some embodiments, the fusion protein mediates a lower increase the level of interferon gamma (IFN-γ) produced by expressing the hIL-12R on the surface relative to the increase in the level of IFN-γ produced in the presence of a suitable control (e.g., a reference hIL-12 fusion protein (e.g., SEQ ID NOS: 371, 372, 383)). In some embodiments, the fusion protein mediates from about a 0.5-1000 fold, 0.5-100 fold, 0.5-10 fold, 0.5-5 fold, 0.5-2 fold, 1-1000 fold, 1-100 fold, 1-10 fold, 1-5 fold, 1-2 fold, 10-1000 fold, or 100-1000 fold lower increase in the level of IFN-γ produced by expressing the hIL-12R on the surface, relative to the increase in the level of IFN-γ produced in the presence of a suitable control (e.g., a reference hIL-12 fusion protein (e.g., SEQ ID NOS: 371, 372, 383)).

In some embodiments, the heterologous moiety comprises or consist of an antibody (or antigen binding domain thereof) and/or one or more Fc region. In some embodiments, the heterologous moiety comprises or consist of an antibody (or antigen binding domain thereof). In some embodiments, the heterologous moiety comprises or consists of a full-length antibody, scFv, (scFv) 2, scFv-Fc, Fab, Fab′, F(ab′) 2, Fab-Fc, a single domain antibody (e.g., VHH), or single domains antibody-Fc (e.g., VHH-Fc. In some embodiments, the antibody (or antigen binding domain thereof) comprises a first variable heavy chain region (VH) that comprises three VH complementarity determining regions (VH CDRs): VH CDR1, VH CDR2, and VH CDR3; and a first variable light chain region (VL) that comprises three VL CDRs: VL CDR1, VL CDR2, and VL CDR3. In some embodiments, the heterologous moiety comprises or consists of a full-length antibody.

In some embodiments, the antibody (or antigen binding domain thereof) specifically binds to a human tumor associated antigen (hTAA).

In some embodiments, the antibody (or antigen binding domain thereof) specifically binds human carbonic anhydrase IX (hCAIX).

In some embodiments, the amino acid sequence of the VH CDR1, VH CDR2, VH CDR3, VL CDR1, VL CDR2, and VL CDR3 each comprises or consists of the amino acid sequence of a VH CDR1, VH CDR2, VH CDR3, VL CDR1, VL CDR2, and VL CDR3 of an antibody set forth in Table 17.

In some embodiments, the amino acid sequence of VH CDR1 comprises the amino acid sequence of SEQ ID NO: 237, or the amino acid sequence of SEQ ID NO: 237 with 1, 2, or 3 amino acid modifications (e.g., substitution, deletion, addition, etc.); the amino acid sequence of VH CDR2 comprises the amino acid sequence of SEQ ID NO: 238, or the amino acid sequence of SEQ ID NO: 238 with 1, 2, or 3 amino acid modifications (e.g., substitution, deletion, addition, etc.); the amino acid sequence of VH CDR3 comprises the amino acid sequence of SEQ ID NO: 239, or the amino acid sequence of SEQ ID NO: 239 with 1, 2, or 3 amino acid modifications (e.g., substitution, deletion, addition, etc.); the amino acid sequence of VL CDR1 comprises the amino acid sequence of SEQ ID NO: 240, or the amino acid sequence of SEQ ID NO: 240 with 1, 2, or 3 amino acid modifications (e.g., substitution, deletion, addition, etc.); the amino acid sequence of VL CDR2 comprises the amino acid sequence of SEQ ID NO: 241 or the amino acid sequence of SEQ ID NO: 242 with 1, 2, or 3 amino acid modifications (e.g., substitution, deletion, addition, etc.); and the amino acid sequence of VL CDR3 comprises the amino acid sequence of SEQ ID NO: 243, or the amino acid sequence of SEQ ID NO: 243 with 1, 2, or 3 amino acid modifications (e.g., substitution, deletion, addition, etc.).

In some embodiments, the amino acid sequence of the VH is at least 85%, 86%, 87%, 88%, 89%, 90%, 91%, 92%, 93%, 94%, 95%, 96%, 97%, 98%, 99%, or 100% identical to the amino acid sequence of any VH polypeptide set forth in Table 17; and the amino acid sequence of the VL comprises or consists of an amino acid sequence at least 85%, 86%, 87%, 88%, 89%, 90%, 91%, 92%, 93%, 94%, 95%, 96%, 97%, 98%, 99%, or 100% identical to the amino acid sequence of any VL polypeptide set forth in Table 17.

In some embodiments, the amino acid sequence of the VH comprises or consists of an amino acid sequence at least 85%, 86%, 87%, 88%, 89%, 90%, 91%, 92%, 93%, 94%, 95%, 96%, 97%, 98%, 99%, or 100% identical to the amino acid sequence of any one of SEQ ID NOS: 7, 246, 256, 264, 274, or 284; and the amino acid sequence of the VL comprises or consists of an amino acid sequence at least 85%, 86%, 87%, 88%, 89%, 90%, 91%, 92%, 93%, 94%, 95%, 96%, 97%, 98%, 99%, or 100% identical to the amino acid sequence of any one of SEQ ID NOS: 12, 247, 257, 265, 275, or 285.

In some embodiments, the amino acid sequence of the VH comprises or consists of an amino acid sequence at least 85%, 86%, 87%, 88%, 89%, 90%, 91%, 92%, 93%, 94%, 95%, 96%, 97%, 98%, 99%, or 100% identical to the amino acid sequence of SEQ ID NO: 7; and the amino acid sequence of the VL comprises or consists of an amino acid sequence at least 85%, 86%, 87%, 88%, 89%, 90%, 91%, 92%, 93%, 94%, 95%, 96%, 97%, 98%, 99%, or 100% identical to the amino acid sequence of SEQ ID NO: 12.

In some embodiments, the amino acid sequence of the VH comprises or consists of an amino acid sequence at least 85%, 86%, 87%, 88%, 89%, 90%, 91%, 92%, 93%, 94%, 95%, 96%, 97%, 98%, 99%, or 100% identical to the amino acid sequence of SEQ ID NO: 7; and the amino acid sequence of the VL comprises or consists of an amino acid sequence at least 85%, 86%, 87%, 88%, 89%, 90%, 91%, 92%, 93%, 94%, 95%, 96%, 97%, 98%, 99%, or 100% identical to the amino acid sequence of SEQ ID NO: 15.

In some embodiments, the fusion protein comprises a first Fc region comprising a CH2 region and a CH3 region; and the second Fc region comprising a CH2 region and a CH3 region. In some embodiments, (a) the first Fc region comprises a CH2 region and a CH3 region; and the second Fc region comprises a CH2 region and a CH3 region; or (b) the first Fc region comprises a hinge region, a CH2 region, and a CH3 region; and the second Fc region comprises a hinge region, a CH2 region, and a CH3 region. In some embodiments, the first Fc region and the second Fc region are each a hIgG1 or hIgG4 Fc region, or functional variant thereof. In some embodiments, the first Fc region and the second Fc region are part of a full-length antibody.

In some embodiments, the CH3 region of the first Fc region and the CH3 region of the second Fc region each comprise at least one amino acid modification that promotes heterodimerization of the first Fc region and the second Fc region.

In some embodiments, the first Fc region comprises an amino acid substitution at amino acid position T366, L368, and Y407, numbering according to EU index of Kabat. In some embodiments, the first Fc region comprises the following amino acid substitutions T366S, L368A, and Y407V, numbering according to EU index of Kabat. In some embodiments, the first Fc region comprises an amino acid substitution at amino acid position Y349, numbering according to EU index of Kabat. In some embodiments, the first Fc region comprises the following amino acid substitution Y349C, numbering according to EU index of Kabat. In some embodiments, the first Fc region comprises an amino acid substitution at amino acid position T366, L368, Y407, and Y349, numbering according to EU index of Kabat. In some embodiments, the first Fc region comprises the following amino acid substitutions T366S, L368A, Y407V, and Y349C, numbering according to EU index of Kabat. In some embodiments, the second Fc region comprises an amino acid substitution at amino acid position T366, numbering according to EU index of Kabat. In some embodiments, the second Fc region comprises the following amino acid substitution T366W, numbering according to EU index of Kabat. In some embodiments, the second Fc region comprises an amino acid substitution at amino acid position S354, numbering according to EU index of Kabat. In some embodiments, the second Fc region comprises the following amino acid substitution S354C, numbering according to EU index of Kabat. In some embodiments, the second Fc region comprises an amino acid substitution at amino acid position T366 and S354, numbering according to EU index of Kabat. In some embodiments, the second Fc region comprises the following amino acid substitutions T366W and S354C, numbering according to EU index of Kabat. In some embodiments, the first Fc region comprises the following amino acid substitutions T366S, L368A, Y407V, and Y349C, and wherein the second Fc region comprises the following amino acid substitutions T366W and S354C, numbering according to EU index of Kabat.

In some embodiments, the second Fc region comprises an amino acid substitution at amino acid position T366, L368, and Y407, numbering according to EU index of Kabat. In some embodiments, the second Fc region comprises the following amino acid substitutions T366S, L368A, and Y407V, numbering according to EU index of Kabat. In some embodiments, the second Fc region comprises an amino acid substitution at amino acid position Y349, numbering according to EU index of Kabat. In some embodiments, the second Fc region comprises the following amino acid substitution Y349C, numbering according to EU index of Kabat. In some embodiments, the second Fc region comprises an amino acid substitution at amino acid position T366, L368, Y407, and Y349, numbering according to EU index of Kabat. In some embodiments, the second Fc region comprises the following amino acid substitutions T366S, L368A, Y407V, and Y349C, numbering according to EU index of Kabat. In some embodiments, the first Fc region comprises an amino acid substitution at amino acid position T366, numbering according to EU index of Kabat. In some embodiments, the first Fc region comprises the following amino acid substitution T366W, numbering according to EU index of Kabat. In some embodiments, the first Fc region comprises an amino acid substitution at amino acid position S354, numbering according to EU index of Kabat. In some embodiments, the first Fc region comprises the following amino acid substitution S354C, numbering according to EU index of Kabat. In some embodiments, the first Fc region comprises an amino acid substitution at amino acid position T366 and S354, numbering according to EU index of Kabat. In some embodiments, the first Fc region comprises the following amino acid substitutions T366W and S354C, numbering according to EU index of Kabat. In some embodiments, the first Fc region comprises the following amino acid substitutions T366S, L368A, Y407V, and Y349C, and wherein the first Fc region comprises the following amino acid substitutions T366W and S354C, numbering according to EU index of Kabat.

In some embodiments, the first Fc region and the second Fc region each comprises at least one amino acid modification (e.g., substitution, deletion, addition) that reduces or eliminates an Fc region effector function compared to a reference Fc region that does not contain the at least one amino acid modification (e.g., substitution, deletion, addition). In some embodiments, the at least one effector function comprises the ability of the Fc region to induce antibody-dependent cellular cytotoxicity (ADCC), antibody-dependent cellular phagocytosis (ADCP), or complement dependent cytotoxicity (CDC), bind an Fc receptor (e.g., an Fcγ receptor), or any combination thereof.

In some embodiments, the first Fc region and the second Fc region each comprises an amino acid substitution at one, two, or three of amino acid positions L234, L235, and/or P329, numbering according to EU index of Kabat. In some embodiments, the first Fc region and the second Fc region each comprises one, two, or three of the following amino acid substitutions: L234A, L235A, and/or P329G or P329A, numbering according to EU index of Kabat. In some embodiments, the first Fc region and the second Fc region each comprise a L234A and L235A amino acid substitution, numbering according to EU index of Kabat. In some embodiments, the first Fc region and the second Fc region each comprise a L234A, L235A, and P329A amino acid substitution, numbering according to EU index of Kabat. In some embodiments, the first Fc region and the second Fc region each comprise a L234A, L235A, and P329G amino acid substitution, numbering according to EU index of Kabat.

In some embodiments, the N-terminus of the hIL-12p40 polypeptide is operably connected to the C-terminus of the first Fc region; and wherein the N-terminus of the hIL-12p35 polypeptide is operably connected to the C-terminus of the second Fc region. In some embodiments, the hIL-12p40 polypeptide is operably connected to the first Fc region via a first peptide linker; and the hIL-12p40 polypeptide is operably connected to the second Fc region via a second peptide linker. In some embodiments, the amino acid sequence of the first peptide linker comprises or consists of the amino acid sequence of a peptide linker set forth in Table 18; and wherein the amino acid sequence of the second peptide linker comprises or consists of the amino acid sequence of a peptide linker set forth in Table 18. In some embodiments, the amino acid sequence of the first peptide linker comprises or consists of the amino acid sequence of any one of SEQ ID NOS: 66-81, 88-303, or 369; and wherein the amino acid sequence of the second peptide linker comprises or consists of the amino acid sequence of any one of SEQ ID NOS: 66-81, 288-303, or 369. In some embodiments, the amino acid sequence of the first peptide linker comprises or consists of the amino acid sequence of SEQ ID NO: 72; and wherein the amino acid sequence of the second peptide linker comprises or consists of the amino acid sequence of SEQ ID NO: 72.

In some embodiments, the N-terminus of the hIL-12p35 polypeptide is operably connected to the C-terminus of the first Fc region; and wherein the N-terminus of the hIL-12p40 polypeptide is operably connected to the C-terminus of the second Fc region. In some embodiments, the hIL-12p40 polypeptide is operably connected to the second Fc region via a first peptide linker; and the hIL-12p40 polypeptide is operably connected to the first Fc region via a second peptide linker. In some embodiments, the amino acid sequence of the first peptide linker comprises or consists of the amino acid sequence of a peptide linker set forth in Table 18; and wherein the amino acid sequence of the second peptide linker comprises or consists of the amino acid sequence of a peptide linker set forth in Table 18. In some embodiments, the amino acid sequence of the first peptide linker comprises or consists of the amino acid sequence of any one of SEQ ID NOS: 66-81, 288-303, or 369; and wherein the amino acid sequence of the second peptide linker comprises or consists of the amino acid sequence of any one of SEQ ID NOS: 66-81, 288-303, or 369. In some embodiments, the amino acid sequence of the first peptide linker comprises or consists of the amino acid sequence of SEQ ID NO: 72; and wherein the amino acid sequence of the second peptide linker comprises or consists of the amino acid sequence of SEQ ID NO: 72.

In some embodiments, the hIL-12p40 polypeptide and the hIL-12p35 polypeptide are operably connected as a schIL-12 polypeptide, and wherein the N-terminus of the schIL-12 polypeptide is operably connected to the C-terminus of the first Fc region or the C-terminus of the second Fc region. In some embodiments, the schIL-12 polypeptide is operably connected to the first Fc region or the second Fc region via a first peptide linker. In some embodiments, the amino acid sequence of the first peptide linker comprises or consists of the amino acid sequence of a peptide linker set forth in Table 18. In some embodiments, the amino acid sequence of the first peptide linker comprises or consists of the amino acid sequence of any one of SEQ ID NOS: 66-81, 288-303, or 369. In some embodiments, the amino acid sequence of the first peptide comprises or consists of the amino acid sequence of SEQ ID NO: 72.

In one aspect, provided herein are fusion proteins comprising a full-length antibody that specifically binds a hTAA comprising: a first light chain comprising from N- to C-terminus a light chain variable region (VL) region and a light chain constant region (CL) region; a first heavy chain comprising from N- to C-terminus a heavy chain variable region (VH) region, a CH1 region, a hinge region, a CH2 region, and a CH3 region; a second heavy chain comprising from N- to C-terminus a VH region, a CH1 region, a hinge region, a CH2 region, and a CH3 region; a second light chain comprising from N- to C-terminus a VL region and a VH region; wherein the first light chain and the first heavy chain associate to form a first antigen binding domain; wherein the second light chain and the second heavy chain associate to form a second antigen binding domain; and wherein the first heavy chain and the second heavy chain associate to form a dimer; a hIL-12p40 polypeptide described herein (e.g., a variant hIL-12p40), a hIL-12p35 polypeptide; wherein the CH3 region of the first heavy chain of the full-length antibody comprises one or more amino acid modification (e.g., substitution) relative to the amino acid sequence of a reference CH3 region that does not contain the one or more amino acid modification (e.g., a wild-type CH3 region, e.g., SEQ ID NO: 122); wherein the CH3 region of the second heavy chain of the of the full-length antibody comprises one or more amino acid modification (e.g., substitution) relative to the amino acid sequence of a reference CH3 region that does not contain the one or more amino acid modification (e.g., a wild-type CH3 region, e.g., SEQ ID NO: 122); wherein the one or more amino acid modification in the CH3 region of the first heavy chain of the full-length antibody is different from the one or more amino acid modification in the CH3 region of the second heavy chain of the full-length antibody; wherein the one or more amino acid modification in the CH3 region of the first heavy chain of the full-length antibody and the one or more amino acid modification in the CH3 region of the second heavy chain of the full-length antibody promote heterodimerization of the first and second heavy chain of the full-length antibody; wherein the N-terminus of the hIL-12p40 polypeptide is operably connected to the C-terminus of the CH3 region of the first heavy chain via a first peptide linker; and wherein the N-terminus of the hIL-12p35 polypeptide is operably connected to the C-terminus of the CH3 region of the second heavy chain via a second peptide linker.

In some embodiments, the hIL-12p40 polypeptide comprises an amino acid sequence (a) at least 85%, 86%, 87%, 88%, 89%, 90%, 91%, 92%, 93%, 94%, 95%, 96%, 97%, 98%, or 99% identical to the amino acid sequence of SEQ ID NO: 33; and (b) comprises or consists of an amino acid substitution at each of amino acid positions W37, F82, and K219, amino acid numbering relative to the amino acid sequence of SEQ ID NO: 32.

In some embodiments, the amino acid sequence of the hIL-12p40 polypeptide comprises or consists of each of the following amino acid substitutions W37A, F82A, and K219A, amino acid numbering relative to the amino acid sequence of SEQ ID NO: 32.

In some embodiments, the amino acid sequence of the hIL-12p40 polypeptide comprises or consists of a set of amino acid substitutions set forth in the amino acid sequence SEQ ID NO: 38 (amino acid substitutions relative to the amino acid sequence of SEQ ID NO: 33); and other than the set of amino acid substitutions, the amino acid sequence of the hIL-12p40 polypeptide is at least 85%, 86%, 87%, 88%, 89%, 90%, 91%, 92%, 93%, 94%, 95%, 96%, 97%, 98%, 99%, or 100% identical to the amino acid sequence of set forth in SEQ ID NOS: 38.

In some embodiments, the amino acid sequence of the hIL-12p40 polypeptide comprises or consists of the amino acid sequence of SEQ ID NO: 38.

In some embodiments, the amino acid sequence of the hIL-12p35 polypeptide is at least 85%, 86%, 87%, 88%, 89%, 90%, 91%, 92%, 93%, 94%, 95%, 96%, 97%, 98%, 99%, or 100% identical to the amino acid sequence of any one of SEQ ID NOS: 31 or 110-114.

In some embodiments, the amino acid sequence of the first peptide linker comprises or consists of the amino acid sequence of SEQ ID NO: 72; and the amino acid sequence of the second peptide linker comprises or consists of the amino acid sequence of SEQ ID NO: 72.

In some embodiments, the first antigen binding domain specifically binds hCAIX, and the second antigen binding domain specifically binds hCAIX.

In some embodiments, the amino acid sequence of VH CDR1 comprises the amino acid sequence of SEQ ID NO: 237, or the amino acid sequence of SEQ ID NO: 237 with 1, 2, or 3 amino acid modifications (e.g., substitution, deletion, addition, etc.); the amino acid sequence of VH CDR2 comprises the amino acid sequence of SEQ ID NO: 238, or the amino acid sequence of SEQ ID NO: 238 with 1, 2, or 3 amino acid modifications (e.g., substitution, deletion, addition, etc.); the amino acid sequence of VH CDR3 comprises the amino acid sequence of SEQ ID NO: 239, or the amino acid sequence of SEQ ID NO: 239 with 1, 2, or 3 amino acid modifications (e.g., substitution, deletion, addition, etc.); the amino acid sequence of VL CDR1 comprises the amino acid sequence of SEQ ID NO: 240, or the amino acid sequence of SEQ ID NO: 240 with 1, 2, or 3 amino acid modifications (e.g., substitution, deletion, addition, etc.); the amino acid sequence of VL CDR2 comprises the amino acid sequence of SEQ ID NO: 241 or the amino acid sequence of SEQ ID NO: 242 with 1, 2, or 3 amino acid modifications (e.g., substitution, deletion, addition, etc.); and the amino acid sequence of VL CDR3 comprises the amino acid sequence of SEQ ID NO: 243, or the amino acid sequence of SEQ ID NO: 243 with 1, 2, or 3 amino acid modifications (e.g., substitution, deletion, addition, etc.).

In some embodiments, the amino acid sequence of the VH comprises or consists of an amino acid sequence at least 85%, 86%, 87%, 88%, 89%, 90%, 91%, 92%, 93%, 94%, 95%, 96%, 97%, 98%, 99%, or 100% identical to the amino acid sequence of SEQ ID NO: 7; and the amino acid sequence of the VL comprises or consists of an amino acid sequence at least 85%, 86%, 87%, 88%, 89%, 90%, 91%, 92%, 93%, 94%, 95%, 96%, 97%, 98%, 99%, or 100% identical to the amino acid sequence of SEQ ID NO: 12.

In some embodiments, the amino acid sequence of the VH comprises or consists of an amino acid sequence at least 85%, 86%, 87%, 88%, 89%, 90%, 91%, 92%, 93%, 94%, 95%, 96%, 97%, 98%, 99%, or 100% identical to the amino acid sequence of SEQ ID NO: 7; and the amino acid sequence of the VL comprises or consists of an amino acid sequence at least 85%, 86%, 87%, 88%, 89%, 90%, 91%, 92%, 93%, 94%, 95%, 96%, 97%, 98%, 99%, or 100% identical to the amino acid sequence of SEQ ID NO: 15.

In some embodiments, the first heavy chain and the second heavy chain each comprises at least one amino acid modification (e.g., substitution, deletion, addition) that reduces or eliminates an Fc region effector function compared to a reference Fc region that does not contain the at least one amino acid modification (e.g., substitution, deletion, addition). In some embodiments, the at least one effector function comprises the ability to induce ADCC, ADCP, or CDC, bind an Fc receptor, or any combination.

In one aspect, provided herein are fusion proteins comprising: a first polypeptide comprising a first light chain comprising from N- to C-terminus a VL region and a CL region; a second polypeptide comprising from N- to C-terminus: (i) a first heavy chain comprising from N- to C-terminus a VH region, a CH1 region, a hinge region, a CH2 region, and a CH3 region; (ii) a first peptide linker, and (iii) the hIL-12p40 polypeptide described herein (e.g., a variant hIL-12p40); third polypeptide comprising from N- to C-terminus: (i) a second heavy chain comprising from N- to C-terminus a VH region, a CH1 region, a hinge region, a CH2 region, and a CH3 region, (ii) a second peptide linker; and (iii) a hIL-12p35 polypeptide; and a fourth polypeptide comprising a second light chain comprising from N- to C-terminus a VL region and a CL region; wherein the VL of the first light chain and the VH of the first heavy chain associate to form a first antigen binding domain that specifically binds a first hTAA; wherein the CH3 region of the first heavy chain comprises one or more amino acid modification (e.g., substitution) relative to the amino acid sequence of a reference CH3 region that does not contain the one or more amino acid modification (e.g., a wild-type CH3 region, e.g., SEQ ID NO: 122); wherein the CH3 region of the second heavy chain comprises one or more amino acid modification (e.g., substitution) relative to the amino acid sequence of a reference CH3 region that does not contain the one or more amino acid modification (e.g., a wild-type CH3 region, e.g., SEQ ID NO: 122); wherein the one or more amino acid modification in the CH3 region of the first heavy chain of the full-length antibody is different from the one or more amino acid modification in the CH3 region of the second heavy chain of the full-length antibody; wherein the one or more amino acid modification in the CH3 region of the first heavy chain of the full-length antibody and the one or more amino acid modification in the CH3 region of the second heavy chain of the full-length antibody promote heterodimerization of the first and second heavy chain of the full-length antibody.

In some embodiments, the hIL-12p40 polypeptide comprises an amino acid sequence (a) at least 85%, 86%, 87%, 88%, 89%, 90%, 91%, 92%, 93%, 94%, 95%, 96%, 97%, 98%, or 99% identical to the amino acid sequence of SEQ ID NO: 33; and (b) comprises or consists of an amino acid substitution at each of amino acid positions W37, F82, and K219, amino acid numbering relative to the amino acid sequence of SEQ ID NO: 32.

In some embodiments, the amino acid sequence of the hIL-12p40 polypeptide comprises or consists of each of the following amino acid substitutions W37A, F82A, and K219A, amino acid numbering relative to the amino acid sequence of SEQ ID NO: 32.

In some embodiments, the amino acid sequence of the hIL-12p40 polypeptide comprises or consists of a set of amino acid substitutions set forth in the amino acid sequence SEQ ID NO: 38 (amino acid substitutions relative to the amino acid sequence of SEQ ID NO: 33); and other than the set of amino acid substitutions, the amino acid sequence of the hIL-12p40 polypeptide is at least 85%, 86%, 87%, 88%, 89%, 90%, 91%, 92%, 93%, 94%, 95%, 96%, 97%, 98%, 99%, or 100% identical to the amino acid sequence of set forth in SEQ ID NOS: 38.

In some embodiments, the amino acid sequence of the hIL-12p40 polypeptide comprises or consists of the amino acid sequence of SEQ ID NO: 38.

In some embodiments, the amino acid sequence of the hIL-12p35 polypeptide is at least 85%, 86%, 87%, 88%, 89%, 90%, 91%, 92%, 93%, 94%, 95%, 96%, 97%, 98%, 99%, or 100% identical to the amino acid sequence of any one of SEQ ID NOS: 31 or 110-114.

In some embodiments, the amino acid sequence of the first peptide linker comprises or consists of the amino acid sequence of SEQ ID NO: 72; and the amino acid sequence of the second peptide linker comprises or consists of the amino acid sequence of SEQ ID NO: 72.

In some embodiments, the full-length antibody specifically binds hCAIX.

In some embodiments, the amino acid sequence of VH CDR1 comprises the amino acid sequence of SEQ ID NO: 237, or the amino acid sequence of SEQ ID NO: 237 with 1, 2, or 3 amino acid modifications (e.g., substitution, deletion, addition, etc.); the amino acid sequence of VH CDR2 comprises the amino acid sequence of SEQ ID NO: 238, or the amino acid sequence of SEQ ID NO: 238 with 1, 2, or 3 amino acid modifications (e.g., substitution, deletion, addition, etc.); the amino acid sequence of VH CDR3 comprises the amino acid sequence of SEQ ID NO: 239, or the amino acid sequence of SEQ ID NO: 239 with 1, 2, or 3 amino acid modifications (e.g., substitution, deletion, addition, etc.); the amino acid sequence of VL CDR1 comprises the amino acid sequence of SEQ ID NO: 240, or the amino acid sequence of SEQ ID NO: 240 with 1, 2, or 3 amino acid modifications (e.g., substitution, deletion, addition, etc.); the amino acid sequence of VL CDR2 comprises the amino acid sequence of SEQ ID NO: 241 or the amino acid sequence of SEQ ID NO: 242 with 1, 2, or 3 amino acid modifications (e.g., substitution, deletion, addition, etc.); and the amino acid sequence of VL CDR3 comprises the amino acid sequence of SEQ ID NO: 243, or the amino acid sequence of SEQ ID NO: 243 with 1, 2, or 3 amino acid modifications (e.g., substitution, deletion, addition, etc.).

In some embodiments, the amino acid sequence of the VH comprises or consists of an amino acid sequence at least 85%, 86%, 87%, 88%, 89%, 90%, 91%, 92%, 93%, 94%, 95%, 96%, 97%, 98%, 99%, or 100% identical to the amino acid sequence of SEQ ID NO: 7; and the amino acid sequence of the VL comprises or consists of an amino acid sequence at least 85%, 86%, 87%, 88%, 89%, 90%, 91%, 92%, 93%, 94%, 95%, 96%, 97%, 98%, 99%, or 100% identical to the amino acid sequence of SEQ ID NO: 12.

In some embodiments, the amino acid sequence of the VH comprises or consists of an amino acid sequence at least 85%, 86%, 87%, 88%, 89%, 90%, 91%, 92%, 93%, 94%, 95%, 96%, 97%, 98%, 99%, or 100% identical to the amino acid sequence of SEQ ID NO: 7; and the amino acid sequence of the VL comprises or consists of an amino acid sequence at least 85%, 86%, 87%, 88%, 89%, 90%, 91%, 92%, 93%, 94%, 95%, 96%, 97%, 98%, 99%, or 100% identical to the amino acid sequence of SEQ ID NO: 15.

In some embodiments, the first heavy chain and the second heavy chain each comprises at least one amino acid modification (e.g., substitution, deletion, addition) that reduces or eliminates an Fc region effector function compared to a reference Fc region that does not contain the at least one amino acid modification (e.g., substitution, deletion, addition). In some embodiments, the at least one effector function comprises the ability to induce ADCC, ADCP, or CDC, bind an Fc receptor, or any combination.

In one aspect, provided herein are antibodies (or antigen binding domain thereof) that specifically bind hCAIX and comprises a VH and VL, wherein the amino acid sequence of the VH is at least 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 3-9; and the amino acid sequence of the VL is at least 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 10-17.

In some embodiments, the amino acid sequence of the VH is at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 7; and the amino acid sequence of the VL is at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 15.

In one aspect, provided herein are polynucleotides encoding a hIL-12p40 polypeptide described herein, a hIL-12p35 polypeptide described herein, a schIL-12 polypeptide described herein, a fusion protein described herein (or one or more polypeptide thereof), or an antibody described herein (or one or more polypeptide thereof). In some embodiments, the polynucleotide is RNA (e.g., mRNA) or DNA. In some embodiments, the polynucleotide is codon optimized.

In one aspect, provided herein are expression vectors comprising a polynucleotide described herein. In some embodiments, the expression vector is a viral vector or a plasmid.

In one aspect, provided herein are host cells comprising a hIL-12p40 polypeptide described herein, a hIL-12p35 polypeptide described herein, a schIL-12 polypeptide described herein, a fusion protein described herein (or one or more polypeptide thereof), an antibody described herein (or one or more polypeptide thereof), a polynucleotide described herein, or an expression vector described herein.

In one aspect, provided herein are carriers comprising a hIL-12p40 polypeptide described herein, a hIL-12p35 polypeptide described herein, a schIL-12 polypeptide described herein, a fusion protein described herein (or one or more polypeptide thereof), an antibody described herein (or one or more polypeptide thereof), a polynucleotide described herein, or an expression vector described herein. In some embodiments, the carrier is a lipid nanoparticle, liposome, lipoplex, or nanoliposome.

In one aspect, provided herein are pharmaceutical compositions a hIL-12p40 polypeptide described herein, a hIL-12p35 polypeptide described herein, a schIL-12 polypeptide described herein, a fusion protein described herein (or one or more polypeptide thereof), an antibody described herein (or one or more polypeptide thereof), a polynucleotide described herein, or an expression vector described herein, a host cell described herein, or a carrier described herein, and a pharmaceutically acceptable excipient.

In one aspect, provided herein are kits comprising a hIL-12p40 polypeptide described herein, a hIL-12p35 polypeptide described herein, a schIL-12 polypeptide described herein, a fusion protein described herein (or one or more polypeptide thereof), an antibody described herein (or one or more polypeptide thereof), a polynucleotide described herein, or an expression vector described herein, a host cell described herein, a carrier described herein, or a pharmaceutical composition described herein.

In one aspect, provided herein are methods of making a hIL-12p40 polypeptide described herein, a hIL-12p35 polypeptide described herein, a schIL-12 polypeptide described herein, a fusion protein described herein (or one or more polypeptide thereof), an antibody described herein (or one or more polypeptide thereof), comprising: introducing into a population of in vitro or ex vivo cells a polynucleotide described herein or an expression vector described herein, culturing the population of cells under conditions sufficient for the population of cells to express the multispecific protein; and optionally isolating and/or purifying the hIL-12p40 polypeptide, hIL-12p35 polypeptide, schIL-12 polypeptide, or fusion protein (or one or more polypeptide thereof).

In one aspect, provided herein are methods of delivering a polypeptide, fusion protein, antibody, polynucleotide, expression vector, host cell, carrier, or pharmaceutical composition to a subject, the method comprising administering a hIL-12p40 polypeptide described herein, a hIL-12p35 polypeptide described herein, a schIL-12 polypeptide described herein, a fusion protein described herein (or one or more polypeptide thereof), an antibody described herein (or one or more polypeptide thereof), a polynucleotide described herein, or an expression vector described herein, a host cell described herein, a carrier described herein, or a pharmaceutical composition described herein to the subject, in an amount and for a time sufficient to deliver the hIL-12p40 polypeptide, the hIL-12p35 polypeptide, schIL-12 polypeptide, fusion protein, polynucleotide, expression vector, host cell, carrier, or pharmaceutical composition to the subject.

A method of stimulating T-cell or NK cell effector function in a subject, the method comprising administering a hIL-12p40 polypeptide described herein, a hIL-12p35 polypeptide described herein, a schIL-12 polypeptide described herein, a fusion protein described herein (or one or more polypeptide thereof), an antibody described herein (or one or more polypeptide thereof), a polynucleotide described herein, or an expression vector described herein, a host cell described herein, a carrier described herein, or a pharmaceutical composition described herein to the subject, in an amount and for a time sufficient to stimulate T-cell or NK cell effector function in the subject.

A method of preventing or treating a cancer in a subject, the method comprising administering a hIL-12p40 polypeptide described herein, a hIL-12p35 polypeptide described herein, a schIL-12 polypeptide described herein, a fusion protein described herein (or one or more polypeptide thereof), an antibody described herein (or one or more polypeptide thereof), a polynucleotide described herein, or an expression vector described herein, a host cell described herein, a carrier described herein, or a pharmaceutical composition described herein to the subject in need thereof, in an amount and for a time sufficient to prevent or treat the cancer in the subject.

In some embodiments, the cancer is a solid tumor. In some embodiments, the cancer lung cancer, central nervous system cancer (e.g., brain cancer or spinal cord cancer, e.g., astrocytoma, glioblastoma), breast cancer, colorectal cancer, colon cancer, rectal cancer, esophageal cancer, kidney cancer, liver cancer, ovarian cancer, pancreatic cancer, prostate cancer, gastric cancer, skin cancer, bladder cancer, uterine cancer, brain cancer, endometrial cancer, lip cancer, oral cancer, mesothelioma, sarcoma, thyroid cancer, thymus cancer, renal cancer, anal cancer, head cancer, neck cancer, or head and neck cancer.

In some embodiments, the cancer is renal cancer (e.g., renal cell carcinoma), bladder cancer, colorectal cancer, small bowel cancer, esophageal/esophagogastric junction (GEJ) cancer, central nervous system cancer (e.g., brain or spinal cord cancer, e.g., glioblastoma), cervical cancer, gastric cancer, lung cancer (e.g., small cell lung cancer), or gastrointestinal cancer.

In one aspect, provided herein are methods of determining the expression of CAIX in cells of a cancer (e.g., a solid cancer) in a subject, the method comprising: obtaining the sample from a subject, wherein the sample does not contain cancer cells (or does not contain a substantial number of cancer cells), and determining the presence or absence of soluble CAIX (or a fragment or variant thereof) in the sample.

In some embodiments, the sample is a blood, serum, or plasma. In some embodiments, the subject is human.

In some embodiments, the cancer is a (e.g., a solid cancer). In some embodiments, the solid cancer is renal cancer (e.g., renal cell carcinoma), bladder cancer, colorectal cancer, small bowel cancer, esophageal/esophagogastric junction (GEJ) cancer, central nervous system cancer (e.g., brain or spinal cord cancer, e.g., glioblastoma), cervical cancer, gastric cancer, lung cancer (e.g., small cell lung cancer), or gastrointestinal cancer.

In one aspect, provided herein are methods of diagnosing a subject with a cancer (e.g., a solid cancer) comprising cancer cells expressing CAIX, the method comprising: obtaining the sample from a subject, wherein the sample does not contain cancer cells (or does not contain a substantial number of cancer cells), determining the presence or absence of soluble CAIX (or a fragment or variant thereof) in the sample; and diagnosing the subject as having a cancer (e.g., a solid cancer) comprising cancer cells expressing CAIX, if soluble CAIX is determined to be present in the sample.

In some embodiments, the sample is a blood, serum, or plasma. In some embodiments, the subject is human.

In some embodiments, the cancer is a (e.g., a solid cancer). In some embodiments, the solid cancer is renal cancer (e.g., renal cell carcinoma), bladder cancer, colorectal cancer, small bowel cancer, esophageal/esophagogastric junction (GEJ) cancer, central nervous system cancer (e.g., brain or spinal cord cancer, e.g., glioblastoma), cervical cancer, gastric cancer, lung cancer (e.g., small cell lung cancer), or gastrointestinal cancer.

In one aspect, provided herein are methods of treating a cancer (e.g., a solid cancer) in a subject, the method comprising: receiving test results that determined the presence of soluble CAIX in a sample from a subject, wherein the sample does not contain cancer cells (or does not contain a substantial number of cancer cells); diagnosing the subject as having a cancer (e.g., a solid cancer) comprising cancer cells expressing CAIX; and administering a hIL-12p40 polypeptide described herein, a hIL-12p35 polypeptide described herein, a schIL-12 polypeptide described herein, a fusion protein described herein (or one or more polypeptide thereof), an antibody described herein (or one or more polypeptide thereof), a polynucleotide described herein, or an expression vector described herein, a host cell described herein, a carrier described herein, or a pharmaceutical composition described herein to the subject in need thereof, in an amount and for a time sufficient to treat the cancer (e.g., a solid cancer) in the subject.

In some embodiments, the sample is a blood, serum, or plasma. In some embodiments, the subject is human.

In some embodiments, the cancer is a (e.g., a solid cancer). In some embodiments, the solid cancer is renal cancer (e.g., renal cell carcinoma), bladder cancer, colorectal cancer, small bowel cancer, esophageal/esophagogastric junction (GEJ) cancer, central nervous system cancer (e.g., brain or spinal cord cancer, e.g., glioblastoma), cervical cancer, gastric cancer, lung cancer (e.g., small cell lung cancer), or gastrointestinal cancer.

### 4. BRIEF DESCRIPTION OF THE FIGURES

FIG. 1 is a graphical depiction of an exemplary antibody (e.g., an anti-CAIX antibody) IL-12 fusion protein described herein. In the specific embodiment depicted, the fusion protein comprises a full-length antibody, IL-12p35 (e.g., an IL-12p35 polypeptide described herein) operably connected to the C-terminus of the first Fc region of the full-length antibody, and IL-12p40 (e.g., an IL-12p40 polypeptide described herein) operably connected to the C-terminus of the second Fc region of the full-length antibody. In the specific embodiment depicted, the first and second Fc regions are heterodimeric, wherein each Fc region comprises at least one amino acid modification (e.g., substitution) that promotes heterodimerization of the first Fc region with the second Fc region. In some embodiments, the first Fc region and the second Fc region each comprise one or more amino acid modification (e.g., substitution) that abolishes or decreases one or more Fc effector function (e.g., antibody-dependent cell-mediated cytotoxicity (ADCC), antibody-dependent cellular phagocytosis (ADCP), complement dependent cytotoxicity (CDC), Fc receptor binding).

FIG. 2 is a graphical depiction of an exemplary antibody (e.g., an anti-CAIX antibody) IL-12 fusion protein described herein. In the specific embodiment depicted, the fusion protein comprises a full-length antibody and a scIL-12 (e.g., a scIL-12 polypeptide described herein) operably connected to the C-terminus of the first Fc region of the full-length antibody. In the specific embodiment depicted, the first and second Fc regions are heterodimeric, wherein each Fc region comprises at least one amino acid modification (e.g., substitution) that promotes heterodimerization of the first Fc region with the second Fc region. In some embodiments, the first Fc region and the second Fc region each comprise one or more amino acid modification (e.g., substitution) that abolishes or decreases one or more Fc effector function (e.g., ADCC, ADCP, CDC, Fc receptor binding).

FIG. 3 is a graphical depiction of an exemplary antibody (e.g., an anti-CAIX antibody) IL-12 fusion protein described herein. In the specific embodiment depicted, the fusion protein comprises two scFvs, wherein one scFv is operably connected to the N-terminus of a first Fc region and the second scFv is operably connected to the N-terminus of a second Fc region; IL-12p35 (e.g., an IL-12p35 polypeptide described herein) operably connected to the C-terminus of the first Fc region; and IL-12p40 (e.g., an IL-12p40 polypeptide described herein) operably connected to the C-terminus of the second Fc region. In the specific embodiment depicted, the first and second Fc regions are heterodimeric, wherein each Fc region comprises at least one amino acid modification (e.g., substitution) that promotes heterodimerization of the first Fc region with the second Fc region. In some embodiments, the first Fc region and the second Fc region each comprise one or more amino acid modification (e.g., substitution) that abolishes or decreases one or more Fc effector function (e.g., ADCC, ADCP, CDC, Fc receptor binding).

FIG. 4 is a graphical depiction of an exemplary antibody (e.g., an anti-CAIX antibody) IL-12 fusion protein described herein. In the specific embodiment depicted, the fusion protein comprises two scFvs, wherein one scFv is operably connected to the N-terminus of a first Fc and the second scFv is operably connected to the N-terminus of a second Fc region; and a scIL-12 (e.g., a sc-IL-12 polypeptide described herein) operably connected to the C-terminus of either the first or second Fc region. In the specific embodiment depicted, the first and second Fc regions are heterodimeric, wherein each Fc region comprises at least one amino acid modification (e.g., substitution) that promotes heterodimerization of the first Fc region with the second Fc region. In some embodiments, the first Fc region and the second Fc region each comprise one or more amino acid modification (e.g., substitution) that abolishes or decreases one or more Fc effector function (e.g., ADCC, ADCP, CDC, Fc receptor binding).

FIG. 5 is a graphical depiction of an exemplary antibody (e.g., an anti-CAIX antibody) IL-12 fusion protein described herein. In the specific embodiment depicted, the fusion protein comprises a full-length antibody and IL-12p35 (e.g., an IL-12p35 polypeptide described herein) operably connected to the C-terminus of one of the first or second Fc regions of the full-length antibody. In the specific embodiment depicted, the first and second Fc regions are heterodimeric, wherein each Fc region comprises at least one amino acid modification (e.g., substitution) that promotes heterodimerization of the first Fc region with the second Fc region. In some embodiments, the first Fc region and the second Fc region each comprise one or more amino acid modification (e.g., substitution) that abolishes or decreases one or more Fc effector function (e.g., ADCC, ADCP, CDC, Fc receptor binding).

FIG. 6 is a graphical depiction of an exemplary antibody (e.g., an anti-CAIX antibody) IL-12 fusion protein described herein. In the specific embodiment depicted, the fusion protein comprises a first DART operably connected to a first Fc region through a coil domain (e.g., a coil domain described herein) and a second DART operably connected to a second Fc region (e.g., a coil domain described herein); IL-12p35 (e.g., an IL-12p35 polypeptide described herein) operably connected to the C-terminus of the first Fc region; and IL-12p40 (e.g., an IL-12p40 polypeptide described herein) operably connected to the C-terminus of the second Fc region. In some embodiments, a non-native disulfide bond is introduced into the heavy chain of the first and second DART. In the specific embodiment depicted, the first and second Fc regions are heterodimeric, wherein each Fc region comprises at least one amino acid modification (e.g., substitution) that promotes heterodimerization of the first Fc region with the second Fc region. In some embodiments, the first Fc region and the second Fc region each comprise one or more amino acid modification (e.g., substitution) that abolishes or decreases one or more Fc effector function (e.g., ADCC, ADCP, CDC, Fc receptor binding).

FIG. 7 is a graphical depiction of an exemplary antibody (e.g., an anti-CAIX antibody) IL-12 fusion protein described herein. In the specific embodiment depicted, the fusion protein comprises two scFvs operably connected in tandem to the N-terminus of a first Fc region; and a scIL-12 (e.g., a scIL-12 polypeptide described herein) operably connected to the N-terminus of a second Fc region. In the specific embodiment depicted, the first and second Fc regions are heterodimeric, wherein each Fc region comprises at least one amino acid modification (e.g., substitution) that promotes heterodimerization of the first Fc region with the second Fc region. In some embodiments, the first Fc region and the second Fc region each comprise one or more amino acid modification (e.g., substitution) that abolishes or decreases one or more Fc effector function (e.g., ADCC, ADCP, CDC, Fc receptor binding).

FIG. 8 is a graphical depiction of an exemplary antibody (e.g., an anti-CAIX antibody) IL-12 fusion protein described herein. In the specific embodiment depicted, the fusion protein comprises two scFvs operably connected in tandem to the N-terminus of a first Fc region; and a scIL-12 (e.g., a scIL-12 polypeptide described herein) operably connected to a single domain antibody, which is operably connected to the N-terminus of a second Fc region. In the specific embodiment depicted, the first and second Fc regions are heterodimeric, wherein each Fc region comprises at least one amino acid modification (e.g., substitution) that promotes heterodimerization of the first Fc region with the second Fc region. In some embodiments, the first Fc region and the second Fc region each comprise one or more amino acid modification (e.g., substitution) that abolishes or decreases one or more Fc effector function (e.g., ADCC, ADCP, CDC, Fc receptor binding).

FIG. 9 is a line graph showing the pSTAT4 signaling (measured through SEAP production) from HEK-Blue cells treated with the indicated construct at the indicated concentrations. Data was analyzed by 4-PL model and presented as Mean±SD of optical density.

FIG. 10A is a line graph showing the level of IFN-γ released by activated T cells in presence of the indicated construct at the indicated concentrations. Data was plotted by 4-PL model and each data point is a mean of triplicate values (mean±SD) from a single experiment. FIG. 10B is a line graph showing the level of IFN-γ released by activated T cells in presence of the indicated construct at the indicated concentrations. Data was plotted by 4-PL model and each data point is a mean of triplicate values (mean±SD) from a single experiment. FIG. 10C is a line graph showing the level of IFN-γ released by activated T cells in presence of the indicated construct at the indicated concentrations. Data was plotted by 4-PL model and each data point is a mean of triplicate values (mean±SD) from a single experiment. FIG. 10D is a line graph showing the level of IFN-γ released by activated T cells in presence of the indicated construct at the indicated concentrations. Data was plotted by 4-PL model and each data point is a mean of triplicate values (mean±SD) from a single experiment. FIG. 10E is a line graph showing the level of IFN-γ released by activated T cells in presence of the indicated construct at the indicated concentrations. Data was plotted by 4-PL model and each data point is a mean of triplicate values (mean±SD) from a single experiment. FIG. 10F is a line graph showing the level of IFN-γ released by activated T cells in presence of the indicated construct at the indicated concentrations. Data was plotted by 4-PL model and each data point is a mean of triplicate values (mean±SD) from a single experiment.

FIG. 11A is a line graph showing the level of IFN-γ released by enriched hIL-2 primed NK cells in presence of the indicated construct at the indicated concentrations. Data was plotted by 4-PL model and each data point is a mean of triplicate values (mean±SD) from a single experiment. FIG. 11B is a line graph showing the level of IFN-γ released by enriched hIL-2 primed NK cells in presence of the indicated construct at the indicated concentrations. Data was plotted by 4-PL model and each data point is a mean of triplicate values (mean±SD) from a single experiment. FIG. 11C is a line graph showing the level of IFN-γ released by enriched hIL-2 primed NK cells in presence of the indicated construct at the indicated concentrations. Data was plotted by 4-PL model and each data point is a mean of triplicate values (mean±SD) from a single experiment. FIG. 11D is a line graph showing the level of IFN-γ released by enriched hIL-2 primed NK cells in presence of the indicated construct at the indicated concentrations. Data was plotted by 4-PL model and each data point is a mean of triplicate values (mean±SD) from a single experiment. FIG. 11E is a line graph showing the level of IFN-γ released by enriched hIL-2 primed NK cells in presence of the indicated construct at the indicated concentrations. Data was plotted by 4-PL model and each data point is a mean of triplicate values (mean±SD) from a single experiment. FIG. 11F is a line graph showing the level of IFN-γ released by enriched hIL-2 primed NK cells in presence of the indicated construct at the indicated concentrations. Data was plotted by 4-PL model and each data point is a mean of triplicate values (mean±SD) from a single experiment.

FIG. 12 is a line graph showing the level of hIL-12R signaling in vitro (measured through SEAP production) from HEK-Blue cells cultured with the indicated constructs at the indicated concentrations. The reported value (mean±SD) is an average of three independent replicates.

FIG. 13 is a line graph showing the level of IFN-γ produced in vitro by PHA stimulated human PBMCs (hPBMCs) cultured with the indicated constructs at the indicated concentrations. Each data point is a mean of triplicate values (mean±SD) and the line graph is representative data from three independent experiments.

FIG. 14 is a line graph showing the level of IFN-γ produced in vitro by IL-2 primed NK cells cultured with the indicated constructs at the indicated concentrations. Each data point is a mean of triplicate values (mean±SD) and the line graph is representative data from three independent experiments.

FIG. 15 is a line graph showing the level of pSTAT4 expressed in vitro by gated CD8+ T cells using anti CD3 and anti CD28 stimulated hPBMCs cultured with the indicated constructs at the indicated concentrations. Each point is data from a single well and the line graph is representative data from three independent experiments.

FIG. 16 is a line graph showing the percent killing of CAIX expressing SNU16 tumor cells by SEB stimulated hPBMCs treated with each of the indicated constructs at the indicated concentrations. Each data point is a value from a single well and the line graph is representative data from two independent experiments.

FIG. 17A is a bar graph showing granzyme B release by SEB stimulated hPBMCs treated with each of the indicated constructs at the indicated concentrations. FIG. 17B is a bar graph showing IFN-γ release by SEB stimulated hPBMCs treated with each of the indicated constructs at the indicated concentrations. FIG. 17C is a bar graph showing TNF-α release by SEB stimulated hPBMCs treated with each of the indicated constructs at the indicated concentrations. FIG. 17D is a bar graph showing IL-10 release by SEB stimulated hPBMCs treated with each of the indicated constructs at the indicated concentrations. FIG. 17E is a bar graph showing MIP-3alpha release by SEB stimulated hPBMCs treated with each of the indicated constructs at the indicated concentrations. FIG. 17F is a bar graph showing CD40-L release by SEB stimulated hPBMCs treated with each of the indicated constructs at the indicated concentrations. FIG. 17G is a bar graph showing Flt3-L release by SEB stimulated hPBMCs treated with each of the indicated constructs at the indicated concentrations. FIG. 17H is a bar graph showing GMCSF release by SEB stimulated hPBMCs treated with each of the indicated constructs at the indicated concentrations. For FIGS. 17A-17H, each bar is a value from single well and the line graph is representative data from an experiment evaluated at 24, 72 and 120 hours.

FIG. 18 is a box plot showing the cytotoxicity of each indicated constructs on HCT116 CAIX expressing spheroids by NK-cells. The cytotoxicity data is pooled data from three donors across four independent experiments. Statistical analysis was done using One way ANOVA, Kruskal wallis test, and all groups were compared to each other (*p value<0.05, **<0.01 is considered as significant).

FIG. 19 is a dot plot (left) and histogram (right) showing the expression of eGFP-CAIX by transfected A549 cells.

FIG. 20 shows bright field and florescence images of CAIX-eGFP fusion protein expressing A459 spheroids, images captured from Cytation 5.

FIG. 21A is a bar graph showing the intensity density of a single CAIX-eGFP fusion protein expressing A459 spheroid treated with the indicated construct. Each bar is a value from single spheroid and the bar graph is representative data from two independent experiments. FIG. 21B is a bar graph showing the cytotoxicity induced by each indicated construct in CAIX-eGFP fusion protein expressing A549 model. Cytotoxicity was calculated considering IgG treated spheroid as 100% viability. Each bar is a value from single spheroid and the bar graph is representative data from two independent experiments.

FIG. 22 is a line graph showing the tumor growth profile of HCT116-CAIX tumors in mice (n=6) treated with the indicated construct. Arrows on the x-axis indicate the dosing days.

FIG. 23 is a line graph showing the tumor growth profile of B16F10 allografts expressing human CAIX (n=6) in hIL-12 and hIL-12 receptor gene knock-in transgenic mice treated with the indicated construct. Arrows on the x-axis indicate the dosing days.

FIG. 24A displays immunohistochemistry images showing CAIX expression in the indicated normal or cancerous tissue. Upper panel: CAIX expression in normal bladder (a), colon (b), cervix (c), kidney (d), and brain (c) (10× magnification). Lower panel: Representative images of heterogeneous CAIX expression in different cancer types: low expression (f, j, n, r, v), moderate expression (g, k, o, s, w), high expression (h, l, p, t, x) (10× magnification). Representative images of heterogeneous CAIX expression in clear cell carcinoma (f, g, h, i), bladder cancer (j, k, l, m), small bowel cancer (n, o, p, q), colorectal cancer (r, s, t, u), and gastric cancer (v, w, x, y). Membranous staining of CAIX in tumor cells (i, m, q, u, y; 30× magnification) of the corresponding tumor types presented in panels h, l, p, t, x, respectively. FIG. 24B is a bar graph showing for each cancer type (Y-axis), the percentage of cases corresponding to CAIX-high (201-300), CAIX-moderate (101-200), CAIX-low (1-100), and CAIX-absent (0) H score category (X-axis). BC (ER+): Breast Cancer (ER+); BLC: Bladder Cancer; BC (Her2+): Breast Cancer (Her2+); CC: Cervical Cancer; CRC: Colorectal Cancer; DLBLC: Diffuse Large B-Cell Lymphoma; EnC: Endometrial Cancer; EsC: Esophageal/GEJ Cancer; GBM: Glioblastoma; GC: Gastric Cancer; GIST: Gastrointestinal Stromal Tumor; HCC: Hepatocellular Carcinoma; HNC: Head & Neck Cancer; MEL: Melanoma; NHL: Non-Hodgkin Lymphoma; NSCLC: Non-Small Cell Lung Cancer; OC: Ovarian Cancer; PC: Pancreatic cancer; PrC: Prostate Cancer; RCC: Renal Cell Carcinoma; SARC: Sarcoma; SBC: Small Bowel Cancer; SCLC: Small Cell Lung Cancer; TC: Thyroid Cancer; TNBC: Triple Negative Breast Cancer.

FIG. 25A displays representative microscopy images showing low (1-199; left panel), medium (200-499, middle panel) and high (>500, right panel) infiltration of lymphocytes in hematoxylin & eosin-stained tumor tissue cores. Red arrowhead: Tumor cell; Green arrowhead: Lymphocyte. FIG. 25B is a bar graph showing for each tumor type (Y-axis), percentage of cases corresponding to high, medium, low, and absent lymphocyte infiltration (X-axis). BC (ER+): Breast Cancer (ER+); BLC: Bladder Cancer; BC (Her2+): Breast Cancer (Her2+); CC: Cervical Cancer; CRC: Colorectal Cancer; DLBLC: Diffuse Large B-Cell Lymphoma; EnC: Endometrial Cancer; EsC: Esophageal/GEJ Cancer; GBM: Glioblastoma; GC: Gastric Cancer; GIST: Gastrointestinal Stromal Tumor; HCC: Hepatocellular Carcinoma; HNC: Head & Neck Cancer; MEL: Melanoma; NSCLC: Non-Small Cell Lung Cancer; OC: Ovarian Cancer; PC: Pancreatic cancer; PrC: Prostate Cancer; RCC: Renal Cell Carcinoma; SARC: Sarcoma; SBC: Small Bowel Cancer; SCLC: Small Cell Lung Cancer; TC: Thyroid Cancer; TNBC: Triple Negative Breast Cancer. FIG. 25C is a graph showing the density of lymphocyte infiltration [average score (0-3), X-axis] in CAIX-expressing tumors (average H score, Y-axis). BC (ER+): Breast Cancer (ER+); BC (Her2+): Breast Cancer (Her2+); BLC: Bladder Cancer; CC: Cervical Cancer; CRC: Colorectal Cancer; DLBLC: Diffuse Large B-Cell Lymphoma; EnC: Endometrial Cancer; EsC: Esophageal/GEJ Cancer; GBM: Glioblastoma; GC: Gastric Cancer; GIST: Gastrointestinal Stromal Tumor; HCC: Hepatocellular Carcinoma; HNC: Head & Neck Cancer; MEL: Melanoma; NSCLC: Non-Small Cell Lung Cancer; OC: Ovarian Cancer; PC: Pancreatic cancer; PrC: Prostate Cancer; RCC: Renal Cell Carcinoma; SARC: Sarcoma; SBC: Small Bowel Cancer; SCLC: Small Cell Lung Cancer; TC: Thyroid Cancer; TNBC: Triple Negative Breast Cancer.

FIG. 26A is a pie chart showing the percentage of different cell types (total 10468 cells) identified by single cell RNA-sequencing on colorectal adenocarcinoma tissues. FIG. 26B are t-distributed stochastic neighbor embedding (t-SNE) plots showing clustering of different cell types in colorectal adenocarcinoma and expression of CA9, IL12RB1, and IL12RB2 in these cell clusters. FIG. 26C is a bar graph showing CA9, IL12RB1, and IL12RB2 gene expression (Y-axis) in cell types (X-axis) identified by single cell RNA-sequencing analysis on colorectal adenocarcinoma tissues. FIG. 26D is a bar graph showing the percentage of a cell type (Y-axis) expressing CA9, IL12RB1, and IL12RB2 in different gene combinations (X-axis).

FIG. 27 is a line graph showing the pSTAT4 signaling (measured through SEAP production) from HEK-Blue cells treated with the indicated constructs at the indicated concentrations. Data was analyzed by 4-PL model and presented as Mean±SD of optical density. BCA307.16 could not be tested at 100 nM concentration due to limitation on stock concentration.

FIG. 28A is a line graph showing the level of IFN-γ released by activated T cells in presence of the indicated hIL-12 construct at the indicated concentrations. Data is plotted by 4-PL model and each point is a mean of triplicate values (mean±SD) from a single experiment. FIG. 28B is a line graph showing the level of IFN-γ released by activated T cells in presence of the indicated hIL-12 construct at the indicated concentrations. Data is plotted by 4-PL model and each point is a mean of triplicate values (mean±SD) from a single experiment. FIG. 28C is a line graph showing the level of IFN-γ released by activated T cells in presence of the indicated hIL-12 construct at the indicated concentrations. Data is plotted by 4-PL model and each point is a mean of triplicate values (mean±SD) from a single experiment.

FIG. 29A is a line graph showing the level of IFN-γ released by enriched hIL-2 primed NK cells in presence of the indicated construct at the indicated concentrations. Data was plotted by 4-PL model and each data point is a mean of triplicate values (mean±SD) from a single experiment. FIG. 29B is a line graph showing the level of IFN-γ released by enriched hIL-2 primed NK cells in presence of the indicated construct at the indicated concentrations. Data was plotted by 4-PL model and each data point is a mean of triplicate values (mean±SD) from a single experiment. FIG. 29C is a line graph showing the level of IFN-γ released by enriched hIL-2 primed NK cells in presence of the indicated construct at the indicated concentrations. Data was plotted by 4-PL model and each data point is a mean of triplicate values (mean±SD) from a single experiment.

FIG. 30 is a dot plot showing Pearson's correlation coefficient analysis between cellular CAIX quantified as an immunohistochemical H Score (X-axis) in tumor tissues and soluble CAIX (pg/mL) in tumor-matched plasma quantified by ELISA (Y-axis) in cancer patients (n=86).

### 5. DETAILED DESCRIPTION

While hIL-12 has been evaluated as a therapeutic for the treatment of cancer, it has showed limited clinical success. To achieve a therapeutic effect, hIL-12 must be dosed at a relatively high level and is highly potent, which has resulted in severe and untenable side effects. Particularly when administered systemically, hIL-12 may cause the activation of immune cells in the bloodstream that express the hIL-12R (e.g., T cells, e.g., CD8+ T cells), creating a systemic inflammatory response that may contribute to the serious side effects associated with hIL-12 based therapy. The inventors have, inter alia, made modified hIL-12 (e.g., hIL-12p40, hIL-12p35) proteins that exhibit decreased potency, while maintaining the ability to mediate tumor cell killing through the activation of immune cells (e.g., T-cells and NK cells). Accordingly, the novel hIL-12 proteins and fusion proteins containing the same (e.g., anti-CAIX fusion proteins) described herein are good candidates for the treatment of diseases (e.g., cancer). As such, the current disclosure provides, inter alia, hIL-12 proteins and fusion proteins (e.g., anti-CAIX fusion proteins) containing the same for use in pharmaceutical compositions for the treatment of diseases (e.g., cancer).

### 5.1 Definitions

The section headings used herein are for organizational purposes only and are not to be construed as limiting the subject matter described.

Unless defined otherwise, all technical and scientific terms used herein have the same meaning as is commonly understood by one of skill in the art to which the claimed subject matter belongs. It is to be understood that the foregoing general description and the following detailed description are exemplary and explanatory only and are not restrictive of any subject matter claimed.

In this application, the use of the singular includes the plural unless specifically stated otherwise. For example, as used in the specification and the appended claims, the singular forms “a,” “an,” and “the” include plural referents unless the context clearly dictates otherwise. Furthermore, use of the term “including” as well as other forms, such as “include,” “includes,” and “included,” is not limiting.

It is understood that wherever aspects are described herein with the language “comprising,” otherwise analogous aspects described in terms of “consisting of” and “consisting essentially of” are also provided.

The term “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

As described herein, any concentration range, percentage range, ratio range or integer range is to be understood to include the value of any integer within the recited range and, when appropriate, fractions thereof (such as one tenth and one hundredth of an integer), unless otherwise indicated.

The terms “about” or “comprising essentially of” refer to a value or composition that is within an acceptable error range for the particular value or composition as determined by one of ordinary skill in the art, which will depend in part on how the value or composition is measured or determined, i.e., the limitations of the measurement system. When particular values or compositions are provided in the application and claims, unless otherwise stated, the meaning of “about” or “comprising essentially of” should be assumed to be within an acceptable error range for that particular value or composition.

Unless otherwise indicated or clear from the context, the use of the terms akin to “first and second,” or “(a) and (b)” or “(i) and (ii)” herein do not denote an order or orientation but are used to identity multiple components of a composition or method. It will be clear from the context to a person of ordinary skill in the art where these terms are intended to denote an order or orientation.

Where proteins and/or polypeptides are described herein, it is understood that polynucleotides (e.g., RNA (e.g., mRNA) or DNA polynucleotides) encoding the protein or polypeptide are also provided herein.

Where proteins, polypeptides, polynucleotides, cells, expression vectors, etc. are described herein, it is understood that isolated forms of the proteins, polypeptides, polynucleotides, cells, expression vectors, etc. are also provided herein.

Where proteins, polypeptides, polynucleotides, etc. are described herein, it is understood that recombinant forms of the proteins, polypeptides, polynucleotides, etc. are also provided herein.

Where polypeptides or sets of polypeptides are described herein, it is understood that proteins comprising the polypeptides or sets of polypeptides folded into their three-dimensional structure (i.e., tertiary or quaternary structure) are also provided herein and vice versa.

As used herein, the term “administering” refers to the physical introduction of an agent, e.g., a therapeutic agent (or a precursor of the therapeutic agent that is metabolized or altered within the body of the subject to produce the therapeutic agent in vitro) to a subject, using any of the various methods and delivery systems known to those skilled in the art. Administering can be performed, for example, once, a plurality of times, and/or over one or more extended periods.

As used herein the term “antibody dependent cell mediated cytotoxicity” or “ADCC” refers to an immune mechanism leading to the lysis of antibody (or an Fc region containing polypeptide or protein) (e.g., an Ig Fc containing fusion protein or polypeptide described herein)-coated target cells by immune effector cells (e.g., NK cells). As used herein, the term “reduced ADCC” and the like refers to either a reduction in the number of target cells that are lysed in a given time, at a given concentration of antibody (or an Ig Fc region containing polypeptide or protein) (e.g., an Fc region containing fusion protein or polypeptide described herein) in the medium surrounding the target cells, by the mechanism of ADCC defined above, and/or an increase in the concentration of antibody (or an Fc region containing polypeptide or protein) (e.g., an Fc containing fusion protein or polypeptide described herein) in the medium surrounding the target cells, required to achieve the lysis of a given number of target cells in a given time, by the mechanism of ADCC defined above. The reduction in ADCC is relative to the ADCC mediated by the same antibody (or an Fc region containing polypeptide or protein) (e.g., an Fc containing fusion protein or polypeptide described herein) produced by the same type of host cells, using the same standard production, purification, formulation and storage methods (which are known to those skilled in the art), but that has not been engineered (e.g., does not comprise one or more amino acid modification, e.g., amino acid substitution, that mediates a decrease in ADCC). For example the reduction in ADCC mediated by an antibody (or an Fc region containing polypeptide or protein) (e.g., an Fc containing fusion protein or polypeptide described herein) comprising in its Fc region an amino acid substitution that reduces ADCC, is relative to the ADCC mediated by the same antibody (or an Fc region containing polypeptide or protein) (e.g., an Fc containing fusion protein or polypeptide described herein) without said amino acid substitution in the Fc region.

As used herein, the term “affinity” refers to the strength of the binding of one protein (e.g., an Antibody) to another protein (e.g., an Antigen). The affinity of a protein is measured by the dissociation constant Kd, defined as [Antibody]×[Antigen]/[Antibody-Antigen] where [Antibody-Antigen] is the molar concentration of the Antibody-Antigen complex, [Antibody] is the molar concentration of the unbound Antibody and [Ligand] is the molar concentration of the unbound Antigen. The affinity constant Ka is defined by 1/Kd. Standard methods of measuring affinity are known to the person of ordinary skill in the art. Exemplary methods of measuring affinity are described herein, see for example, § 5.2.2.

As used herein, the term “antibody” or “antibodies” is used in the broadest sense and encompasses various immunoglobulin (Ig) (e.g., human Ig (hIg)) structures, including, but not limited to monoclonal antibodies, polyclonal antibodies, multispecific (e.g., bispecific, trispecific) antibodies, and antibody fragments so long as they exhibit the desired antigen-binding activity (i.e., antigen binding fragments or variants). The term antibody thus includes, for example, full-length antibodies; antigen-binding fragments of full-length antibodies; molecules comprising antibody CDRs, VH regions, and/or VL regions; and antibody-like scaffolds (e.g., fibronectins). Examples of antibodies include, without limitation, monoclonal antibodies, polyclonal antibodies, monospecific antibodies, multispecific antibodies, human antibodies, humanized antibodies, chimeric antibodies, camelized antibodies, intrabodies, affybodies, diabodies, tribodies, heteroconjugate antibodies, antibody-drug conjugates, single domain antibodies (e.g., VHH, (VHH)₂), single chain antibodies, single-chain Fvs (scFv; (scFv)₂), Fab fragments (e.g., Fab, single chain Fab (scFab), F(ab′)₂ fragments, disulfide-linked Fvs (sdFv), Fc fusions (e.g., Fab-Fc, scFv-Fc, VHH-Fc, (scFv)₂-Fc, (VHH)₂-Fc), and antigen-binding fragments of any of the above, and conjugates or fusion proteins comprising any of the above. Antibodies can be of Ig isotype (e.g., IgG, IgE, IgM, IgD, or IgA), any class (e.g., IgG₁, IgG₂, IgG₃, IgG₄, IgA₁ or IgA₂), or any subclass (e.g., IgG₂a or IgG₂b) of Ig). In certain embodiments, antibodies described herein are IgG antibodies, or a class (e.g., human IgG₁ or IgG₄) or subclass thereof. In some embodiments, the antibody is a human, humanized, or chimeric IgG₁ or IgG₄ monoclonal antibody. In some embodiments, the term antibodies refers to a monoclonal or polyclonal antibody population. Antibodies described herein can be produced by any standard methos known in the art, e.g., recombinant production in host cells, see, e.g., § 5.4; or synthetic production.

“Antibody-like scaffolds” are known in the art, for example, fibronectin and designed ankyrin repeat proteins (DARPins) have been used as alternative scaffolds for antigen-binding domains, see, e.g., Gebauer and Skerra, Engineered protein scaffolds as next-generation antibody therapeutics. Curr Opin Chem Biol 13:245-255 (2009) and Stumpp et al., Darpins: A new generation of protein therapeutics. Drug Discovery Today 13:695-701 (2008), the full contents of each of which is incorporated herein by reference for all purposes. Exemplary antibody-like scaffold proteins include, but are not limited to, lipocalins (Anticalin), Protein A-derived molecules such as Z-domains of Protein A (Affibody), an A-domain (Avimer/Maxibody), a serum transferrin (trans-body); a designed ankyrin repeat protein (DARPin), VNAR fragments, a fibronectin (AdNectin), a C-type lectin domain (Tetranectin); a variable domain of a new antigen receptor beta-lactamase (VNAR fragments), a human gamma-crystallin or ubiquitin (Affilin molecules); a kunitz type domain of human protease inhibitors, microbodies such as the proteins from the knottin family, peptide aptamers and fibronectin (adnectin).

The term “antigen binding domain” refers to a polypeptide or protein, or the portion of a polypeptide or protein, that is capable of specifically binding to an antigen. Exemplary antigen binding domains include, but are not limited to, single domain antibodies (e.g., VHH, (VHH)₂), single-chain Fvs (e.g., scFv; (scFv)₂), Fab fragments (e.g., Fab, single chain Fab (scFab), F(ab′)₂), and disulfide-linked Fvs (sdFv). The antigen binding domain can be part of a larger polypeptide or protein, e.g., a full-length antibody, an Fc fusion. In some embodiments, the antigen binding domain is part of a full-length antibody. In some embodiments, the antigen binding domain is operably connected to an Fc region. When an antigen binding domain is referred to using the target protein or polypeptide, the term “antigen” may be replaced with the name of the target protein or antigen. For example, an antigen binding domain that specifically binds hCAIX may also be referred to herein as a “hCAIX binding domain.”

The terms “cancer” and “tumor” are used interchangeably herein and refer to a broad group of various diseases characterized by the uncontrolled growth of abnormal cells in the body. Unregulated cell division and growth results in the formation of malignant tumors that can invade neighboring tissues and may also metastasize to distant parts of the body through, e.g., the lymphatic system or bloodstream.

As used herein, the term “CDR” or “complementarity determining region” refers to the noncontiguous antigen combining sites found within the variable region of both heavy and light chain polypeptides. These particular regions have been described by Kabat et al., J. Biol. Chem. 252, 6609-6616 (1977) and Kabat et al., Sequences of protein of immunological interest. (1991), the entire contents of each of which is incorporated herein by reference for all purposes. Unless otherwise specified, the term “CDR” is a CDR as defined by Kabat et al., J. Biol. Chem. 252, 6609-6616 (1977) and Kabat et al., Sequences of protein of immunological interest. (1991).

The terms “CH1” and “CH1 region” are used interchangeably herein and refer to the first constant region of an immunoglobulin heavy chain. The amino acid sequence of an exemplary reference hIgG1 CH1 region is set forth in SEQ ID NO: 119; and the amino acid sequence of an exemplary reference hIgG4 CH1 region is set forth in SEQ ID NO: 132.

The terms “CH2” and “CH2 region” are used interchangeably herein and refer to the second constant region of an immunoglobulin heavy chain. The amino acid sequence of an exemplary reference hIgG1 CH2 region is set forth in SEQ ID NO: 121; and the amino acid sequence of an exemplary reference hIgG4 CH2 region is set forth in SEQ ID NO: 134.

The terms “CH3” and “CH3 region” are used interchangeably herein and refer to the third constant region of an immunoglobulin heavy chain. The amino acid sequence of an exemplary reference hIgG1 CH3 region is set forth in SEQ ID NO: 122; and the amino acid sequence of an exemplary reference hIgG4 CH3 region is set forth in SEQ ID NO: 135.

The terms “constant region” and “constant domain” are used interchangeably herein and refer to a carboxyl terminal portion of a light and/or heavy chain of a full-length antibody which is not directly involved in binding of an antibody to antigen, but which can exhibit various effector functions, such as interaction with an Ig Fc receptor (e.g., Fc gamma receptor). The constant region of an Ig molecule generally has a more conserved amino acid sequence relative to an Ig variable domain.

As used herein, the term “derived from,” with reference to a polynucleotide refers to a polynucleotide that has at least 70% (e.g., at least 85%) sequence identity to a reference polynucleotide (e.g., a naturally occurring polynucleotide) or a fragment thereof. The term “derived from,” with reference to a polypeptide or protein refers to a polypeptide or protein that comprises an amino acid sequence that has at least 70% (e.g., at least 85%) sequence identity to the amino acid sequence of a reference polypeptide or protein (e.g., a naturally occurring polypeptide or protein). The term “derived from” as used herein does not denote any specific process or method for obtaining the polynucleotide, polypeptide, or protein. For example, the polynucleotide, polypeptide, or protein can be recombinant produced or chemically synthesized.

As used herein, the term “diagnosing” or “diagnosis” refers to a determination of the presence, absence, severity, or course of treatment of a disease (e.g., a cancer, e.g., a cancer comprising cancer cells expressing CAIX). The term “diagnosing” encompasses an initial determination as well as subsequent determinations (e.g., monitoring) after the initial determination.

As used herein, the term “disease” refers to any abnormal condition that impairs physiological function. The term is used broadly to encompass any disorder, illness, abnormality, pathology, sickness, condition, or syndrome in which physiological function is impaired, irrespective of the nature of the etiology.

The terms “hinge” or “hinge region” are used interchangeably herein and refer to the hinge region of an immunoglobulin heavy chain. The amino acid sequence of an exemplary reference hIgG1 hinge region is set forth in SEQ ID NO: 120; and the amino acid sequence of an exemplary reference hIgG4 hinge region is set forth in SEQ ID NO: 133.

The terms “DNA” and “polydeoxyribonucleotide” are used interchangeably herein and refer to macromolecules that include multiple deoxyribonucleotides that are polymerized via phosphodiester bonds. Deoxyribonucleotides are nucleotides in which the sugar is deoxyribose.

The term “effector function” when used in reference to an antibody refers to those biological activities attributable to the Fc region of an antibody, which therefore vary with the antibody isotype. Antibody effector functions include, but are not limited to, antibody-dependent cell-mediated cytotoxicity (ADCC), antibody-dependent cellular phagocytosis (ADCP), complement dependent cytotoxicity (CDC), Fc receptor binding (e.g., FcγRI, FcγRIIa, FcγRIIc, FcγRIIIa, and/or FcγRIIIb (e.g., FcγRI, FcγIIa, and/or FcγIIIa)), and Clq binding.

As used herein, the term “EU numbering system” refers to the EU numbering convention for the constant regions of an antibody, as described in Edelman, G. M. et al., Proc. Natl. Acad. USA, 63, 78-85 (1969) and Kabat et al, Sequences of Proteins of Immunological Interest, U.S. Dept. Health and Human Services, 5th edition, 1991, the entire contents of each of which is incorporated herein by reference for all purposes.

As used herein, the term “Fab” refers to an antigen binding domain that comprises a Fab heavy chain that comprises from N- to C-terminus a VH region and a CH1 region; and a light chain comprising from N- to C-terminus a VL region and a CL region; and wherein the Fab heavy chain and the light chain associate to form an antigen binding domain.

The term “Fab-Fc” as used herein refers to an antibody that comprises a Fab operably linked to an Fc region. For example, a full-length antibody comprises a first Fab operably connected to a first Fc region and a second Fab operably connected to a second Fc region.

As used herein, the term “Fc region” refers to the C-terminal region of a hIg heavy chain that comprises from N- to C-terminus at least a CH2 region operably connected to a CH3 region. In some embodiments, the Fc region comprises an Ig hinge region or at least a portion of an Ig hinge region operably connected to the N-terminus of the CH2 region. In some embodiments, the Fc region is engineered relative to a reference Fc region (e.g., comprises one or more amino acid modification), see, e.g., § 5.3.2.1. Additional examples of proteins with engineered Fc regions can be found in Saunders 2019 (K. O. Saunders, “Conceptual Approaches to Modulating Antibody Effector Functions and Circulation Half-Life,” 2019, Frontiers in Immunology, V. 10, Art. 1296, pp. 1-20, the entire contents of which is incorporated herein by reference for all purposes).

As used herein, the term “Fc modified fusion protein or polypeptide” refers to a fusion polypeptide or protein comprising an Fc region, wherein the Fc region is modified (e.g., comprises one or more amino acid modification (e.g., one or more amino acid substitution, deletion, or addition).

As used herein, the terms “first” and “second” with respect to Fc regions etc., are used for convenience of distinguishing when there is more than one of each type of moiety. Use of these terms is not intended to confer a specific order or orientation in the fusion protein unless explicitly so stated.

As used herein, the term “framework region” or “FR region” refers to the amino acid residues that are part of the variable region of an antibody, but are not part of the CDRs (e.g., using the Kabat definition of CDRs).

As used herein, the term “full-length antibody” refers to an antibody having a structure substantially similar to a native antibody structure (i) a first Ig light chain comprising from N- to C-terminus a light chain variable region (VL) region and a light chain constant region (CL) region; (ii) a first Ig heavy chain comprising from N- to C-terminus a heavy chain variable region (VH) region, a CH1 region, a hinge region, a CH2 region, and a CH3 region; (iii) a second Ig heavy chain comprising from N- to C-terminus a VH region, a CH1 region, a hinge region, a CH2 region, and a CH3 region; (iv) a second Ig light chain comprising from N- to C-terminus a VL region and a VH region; wherein said first light chain and said first heavy chain associate to form a first antigen binding domain; wherein said second light chain and said second heavy chain associate to form a second antigen binding domain; and wherein said first heavy chain and said second heavy chain associate to form a dimer. In some embodiments, the two heavy chains comprise a substantially identical amino acid sequence; and the two light chains comprise a substantially identical amino acid sequence. The amino acid sequence of the two heavy chains can be different, e.g., contain one or more amino acid modification that promotes heterodimerization of the two heavy chains. In some embodiments, the two heavy chains comprise a substantially identical amino acid sequence except for one or more amino acid modifications that promote heterodimerization of the correct heavy chains (e.g., as described herein); and the two light chains comprise a substantially identical amino acid sequence. Antibody chains may be substantially identical but not entirely identical if they differ due to post-translational modifications, such as C-terminal cleavage of lysine residues, alternative glycosylation patterns, etc. The amino acid sequence of any one of the chains of a full-length antibody may contain one or more amino acid modifications relative to a reference (e.g., wild type antibody sequence).

The term “functional variant” as used herein in reference to a polypeptide or protein refers to a polypeptide or protein that comprises at least one but no more than 15%, not more than 12%, no more than 10%, no more than 8% amino acid variation (e.g., substitution, deletion, addition) compared to the amino acid sequence of a reference polypeptide or protein, wherein the polypeptide or protein retains at least one particular function of the reference polypeptide or protein. Not all functions of the reference polypeptide or protein (e.g., wild type) need be retained by the functional variant of the protein. In some instances, one or more functions are selectively reduced or eliminated. In some embodiments, the reference polypeptide or protein is a wild type protein. For example, a functional variant of a hIL-12p40 polypeptide or protein can refer to a hIL-12p40 protein comprising an amino acid substitution as compared to a reference hIL-12p40 protein (e.g., wild type) that retains the ability to specifically bind hIL-12R.

The term “functional fragment” as used herein in reference to a polypeptide or protein refers to a fragment of a reference polypeptide or protein that retains at least one particular function. Not all functions of the reference polypeptide or protein need be retained by a functional fragment of the polypeptide or protein. In some instances, one or more functions are selectively reduced or eliminated. In some embodiments, the reference polypeptide or protein is a wild type protein. For example, a functional fragment of hIL-12p40 can refer to a fragment of hIL-12p40 that retains the ability to specifically bind IL-12R.

As used herein, the term “fuse” and grammatical equivalents thereof refer to the operable connection of at least a one polypeptide derived from a first polypeptide to another polypeptide derived from a second polypeptide, wherein the first and second polypeptides are different. The term fuse encompasses both a direct connection of the at least two polypeptides through a peptide bond, and the indirect connection through a linker (e.g., a peptide linker).

As used herein, the term “fusion polypeptide” or “fusion protein” and grammatical equivalents thereof refers to a polypeptide or protein that comprises at least one polypeptide derived from a first polypeptide operably connected to another polypeptide derived from a second polypeptide, wherein the first and second polypeptides are different. The at least two polypeptides of the fusion polypeptide or protein can be directly operably connected through a peptide bond; or can be indirectly operably connected through a linker (e.g., a peptide linker). Therefore, for example, the term fusion polypeptide encompasses embodiments, wherein Polypeptide A is directly operably connected to Polypeptide B through a peptide bond (Polypeptide A-Polypeptide B), and embodiments, wherein Polypeptide A is operably connected to Polypeptide B through a peptide linker (Polypeptide A-peptide linker-Polypeptide B).

As used herein, the term “heavy chain” refers to the portion of an immunoglobulin (e.g., a human Ig) that typically comprises from N- to C-terminus a heavy chain variable region (VH), a CH1 region, a hinge region, a CH2 region, and a CH3 region. The constant regions of the heavy chain (i.e., the CH1 region, the hinge region, the CH2 region, and the CH3 region) can be any distinct isotype, for example, human alpha (α), delta (δ), epsilon (ε), gamma (γ), and mu (μ), based on the amino acid sequence of the constant domain, which give rise to the hIgA, hIgD, IgE, hIgG, and hIgM classes of human antibodies, respectively, including subclasses of hIgG, e.g., hIgG₁, hIgG₂, hIgG₃, and hIgG₄. As used herein, the term “heavy chain” when used in reference to a human antibody can refer to any distinct type, e.g., alpha (α), delta (δ), epsilon (ε), gamma (γ), and mu (μ), based on the amino acid sequence of the constant domain, which give rise to human IgA, IgD, IgE, IgG, and IgM classes of antibodies, respectively, including subclasses of human IgG, e.g., IgG₁, IgG₂, IgG₃, and IgG₄.

As used herein, the term “half-life extension moiety” refers to a moiety (e.g., small molecule, polypeptide, polynucleotide, carbohydrate, lipid, synthetic polymer (e.g., polymers of PEG), etc.) that when conjugated or otherwise operably connected (e.g., fused) to a polypeptide or protein (the subject polypeptide or protein), increases the half-life of the subject polypeptide or protein in vitro when administered to a subject (e.g., a human subject). The pharmacokinetic properties of the polypeptide or protein can be evaluated utilizing in vitro models known in the art.

As used herein, the term “half-life extension polypeptide” or “half-life extension protein” refers to a polypeptide that when operably connected to another polypeptide (the subject polypeptide or protein), increases the half-life of the subject polypeptide in vitro when administered to a subject (e.g., a human subject). The pharmacokinetic properties of the polypeptide or protein can be evaluated utilizing in vitro models known in the art.

As used herein, the term “heterologous”, when used to describe a first element in reference to a second element means that the first element and second element do not exist in nature disposed as described. For example, a polypeptide comprising a “heterologous moiety” means a polypeptide that is joined to a moiety (e.g., small molecule, polypeptide, polynucleotide, carbohydrate, lipid, synthetic polymer (e.g., polymers of PEG), etc.) that is not joined to the polypeptide in nature. For example, a non-limiting example of a heterologous moiety is a heterologous polypeptide (as defined herein).

As used, herein the term “heterologous signal peptide” refers to a signal peptide that is not operably connected to a subject polypeptide or protein in nature. For example, in reference to a polypeptide comprising a signal peptide from human IL-2 (hIL-2) operably connected to hIL-12p40, the hIL-2 signal peptide would constitute a heterologous signal peptide.

As used herein, the term “homologous signal peptide” refers to a signal peptide that is operably connected to a subject polypeptide or protein in nature. For example, in reference to a polypeptide comprising a signal peptide from human IL-2 operably connected to hIL-2, the hIL-2 signal peptide would constitute a homologous signal peptide.

The term “human carbonic anhydrase IX” or “CAIX” refers to human carbonic anhydrase transmembrane dimeric metalloenzyme that facilitates acid secretion in the gastrointestinal tract. CAIX is also referred to in the art as “Carbonate dehydratase IX,” “Carbonic anhydrase 9,” “CA9,” and “CA-IX.” The amino acid sequence of an exemplary reference mature hCAIX protein can be found under Uniprot Accession Number Q16790, and herein set forth in SEQ ID NO: 1.

As used herein, the term “human tumor associated antigen” or “hTAA” refers to a protein that is expressed on the surface of a human cancer cell that allows recruitment of a multispecific protein described herein to the human cancer cell. In some embodiments, the tumor associated antigen is expressed by both normal cells and cancer cells. In some embodiments, the tumor associated antigen is overexpressed by a cancer cell in comparison to a normal cell, for example, 1-fold over expression, 2-fold overexpression, 3-fold overexpression or more in comparison to a normal cell. In some embodiments, the tumor associated antigen is inappropriately synthesized by the cancer cell, for example, a protein that contains amino acid modifications (e.g., amino acid deletions, additions, and/or substitutions), in comparison to the protein expressed by a normal cell. In some embodiments, the tumor associated antigen is only expressed by the cancer cell and not expressed at detectable level by normal cells. Methods to identify and verify tumor-associated proteins are known to a skilled person and described in the literature (see, e.g., Bornstein, AAPS J. (2015), vol. 17 (3), p. 525-534; Hong et al., BMC Syst Biol. (2018), vol. 12 (Suppl 2), p. 17.

The term “human interleukin 12” or “hIL-12” refers to a human IL-12 protein or polypeptide.

The terms “interleukin 12” and “IL-12” are used interchangeably herein and are intended to mean and encompass functional IL-12 protein complexes comprising an IL-12p35 subunit and an IL-12p40 subunit. In some embodiments, the IL-12p35 subunit and the IL-12p40 subunit are operably connected in a single polypeptide chain, e.g., a single chain IL-12 (scIL-12) (e.g., a scIL-12 described herein). In some embodiments, the IL-12p35 subunit and the IL-12p40 subunit are not operably connected in a single polypeptide chain but are encoded by two separate polypeptides.

The terms “single chain IL-12,” and “scIL-12” are used interchangeably herein and refer to forms of IL-12, which have been engineered to express the IL-12p40 polypeptide fused either directly or indirectly via a peptide linker, to the IL-12p35 polypeptide such that the IL-12p40/IL-12p35 molecule is produced as a single polypeptide chain (i.e., a fusion polypeptide). The scIL-12 can be configured in either order such that the single polypeptide is produced beginning with the IL-12p40 polypeptide as the amino-terminal (“N-terminal”) portion fused directly or indirectly via a peptide linker to the IL-12p35 polypeptide as the carboxyl-terminal (“C-terminal”) portion of the scIL-12. This configuration may be represented by a shorthand designation as “IL-12p40-linker-IL-12p35”, when a peptide linker is utilized. Conversely, in a scIL-12 construct, the IL-12p35 polypeptide can also be the N-terminal portion fused directly or via a peptide linker to IL-12p40 as the C-terminal portion of the scIL-12. This configuration may be represented by a shorthand designation as “IL-12p35-linker-IL-12p40”, when a peptide linker is utilized.

The term “hIL-12p35” as used herein refers to the human alpha subunit of the heterodimeric IL-12 protein. The amino acid sequence of an exemplary reference IL-12p35 can be found under Uniprot Accession Number P29459, and herein set forth in SEQ ID NO: 30. For purposes of the instant disclosure, the numbering of all amino acids (and e.g., amino acid substitutions) of hIL-12p35 polypeptides described herein is set out relative to the amino acid sequence of the immature form of hIL-12p35 (i.e., SEQ ID NO: 30), that contains the native signal peptide. As described herein, amino acids 1-22 of SEQ ID NO: 30 are the native signal peptide, which is cleaved in vivo to form the mature protein (SEQ ID NO: 31). The use of the immature form of hIL-12p35 to designate amino acid numbering is for consistency only and does not limit the scope of embodiments utilizing this numbering to polypeptides that contain the signal peptide of hIL-12p35. For example, a hIL-12p35 polypeptide described herein as comprising the amino acid sequence of SEQ ID NO: 31 (mature form of hIL-12p35) with a Y189A amino acid substitution does not require the signal peptide of hIL-12p35, although the numbering of amino acid position Y189 is based on the immature form of the protein. It common in the art to utilize the mature form of a protein to produce variants and fusion proteins. A person of ordinary skill in art can easily determine the amino acid position in the mature form of hIL-12p35 (SEQ ID NO: 31) based on the amino acid numbering relative to the immature form of hIL-12p35. As set forth above, amino acids 1-22 of the immature form of the hIL-12p35 protein are the signal sequence. Therefore, an amino acid position of a particular amino acid in the mature form of a hIL-12p35 protein can be determined from the amino acid position of the particular amino acid designated relative to the immature form of hIL-12p35 by subtracting 22. For example, the amino acid position Y189 (numbering relative to SEQ ID NO: 30) would correspond to amino acid position Y167 in the mature form of the protein (SEQ ID NO: 32).

The term “hIL-12p40” as used herein refers to the human beta subunit of the heterodimeric IL-12 protein. The amino acid sequence of an exemplary reference IL-12p40 can be found under Uniprot Accession Number P29460, and herein set forth in SEQ ID NO: 32. For purposes of the instant disclosure, the numbering of all amino acids (and e.g., amino acid substitutions) of hIL-12p40 polypeptides described herein is set out relative to the amino acid sequence of the immature form of hIL-12p40 (i.e., SEQ ID NO: 32), that contains the native signal peptide. As described herein, amino acids 1-22 of SEQ ID NO: 32 are the native signal peptide, which is cleaved in vivo to form the mature protein (SEQ ID NO: 33). The use of the immature form of hIL-12p40 to designate amino acid numbering is for consistency only and does not limit the scope of embodiments utilizing this numbering to polypeptides that contain the signal peptide of hIL-12p40. For example, a hIL-12p40 polypeptide described herein as comprising the amino acid sequence of SEQ ID NO: 33 (mature form of hIL-12p40) with a W37A amino acid substitution does not require the signal peptide of hIL-12, although the numbering of amino acid position W37 is based on the immature form of the protein. It common in the art to utilize the mature form of a protein to produce variants and fusion proteins. A person of ordinary skill in art can easily determine the amino acid position in the mature form of hIL-12p40 (SEQ ID NO: 33) based on the amino acid numbering relative to the immature form of hIL-12p40. As set forth above, amino acids 1-22 of the immature form of the hIL-12p40 protein are the signal sequence. Therefore, an amino acid position of a particular amino acid in the mature form of a hIL-12p40 protein can be determined from the amino acid position of the particular amino acid designated relative to the immature form of hIL-12p40 by subtracting 22. For example, the amino acid position W37 (numbering relative to SEQ ID NO: 32) would correspond to amino acid position W15 in the mature form of the protein (SEQ ID NO: 33).

As used herein, the term “isolated” with reference to a polypeptide, protein, or polynucleotide refers to a polypeptide, protein, or polynucleotide that is substantially free of other cellular components or other contaminants with which it is associated in the natural state.

As used herein, the term “Kabat numbering system” refers to the Kabat numbering convention for variable regions of an antibody, see, e.g., Kabat et al, Sequences of Proteins of Immunological Interest, U.S. Dept. Health and Services, 5th edition, 1991, the entire contents of which are incorporated herein by reference for all purposes. Unless otherwise noted, numbering of the variable regions of an antibody are denoted according to the Kabat numbering system.

As used herein, the term “linker” refers to a linkage between two elements (e.g., polypeptide or protein domains). A linker can be a covalent bond or a peptide linker. The term “bond” refers to a chemical bond, (e.g., an amide bind, a disulfide bond, or any kind of bond created from a chemical reaction (e.g., chemical conjugation)). The term “peptide linker” refers to an amino acid or polypeptide that may be employed to link two polypeptide or protein domains. In some embodiments, a peptide linker may be used to provide space and/or flexibility between the two polypeptide or protein domains.

As used herein, the term “light chain” refers to the portion of an immunoglobulin (e.g., a human immunoglobulin) that comprises from N- to C-terminus a light chain variable region (VL) operably connected to a light chain constant region (CL). The CL can be any distinct type, e.g., kappa (κ) or lambda (λ) based on the amino acid sequence of the CL. In some embodiments, the multispecific proteins described herein comprise one or more light chain.

As used herein, the term “messenger RNA” or “mRNA” refers to any RNA that encodes at least one peptide or protein and can be translated to produce the encoded peptide or protein in vitro, in vitro, in situ or ex vivo.

As used herein, the term “modification,” with reference to a polynucleotide, refers to a polynucleotide that comprises at least one substitution, alteration, inversion, addition, or deletion of nucleotide compared to a reference polynucleotide (e.g., one or more amino acid substitutions). Modifications can include the inclusion of non-naturally occurring nucleotide residues. As used herein, the term “modification,” with reference to an amino acid sequence refers to an amino acid sequence that comprises at least one substitution, alteration, inversion, addition, or deletion of an amino acid residue compared to a reference amino acid sequence. Modifications can include the inclusion of non-naturally occurring amino acid residues. Naturally occurring amino acid derivatives are not considered modified amino acids for purposes of determining percent identity of two amino acid sequences. For example, a naturally occurring modification of a glutamate amino acid residue to a pyroglutamate amino acid residue would not be considered an amino acid modification for purposes of determining percent identity of two amino acid sequences. Further, for example, a naturally occurring modification of a glutamate amino acid residue to a pyroglutamate amino acid residue would not be considered an amino acid “modification” as defined herein.

A “modification that promotes heterodimerization of a first Fc region and a second Fc region” (or similar phrasing) is a manipulation of the peptide backbone or the post-translational modifications of an Fc region that reduces or prevents the association of a polypeptide comprising the Fc region with an identical polypeptide to form a homodimer. A modification promoting association as used herein particularly includes separate modifications made to each of the two Fc regions desired to associate (i.e., a first Fc region and a second Fc region), wherein the modifications are complementary to each other so as to promote association of the two Fc regions. For example, a modification promoting association may alter the structure or charge of one or both of the Fc regions so as to make their association sterically or electrostatically favorable, respectively. Thus, heterodimerization occurs between a polypeptide comprising the first Fc region and a polypeptide comprising the second Fc region, which might be non-identical in the sense that further components fused to each of the Fc regions (e.g., antigen binding domains) are not the same. In some embodiments the modification promoting association comprises an amino acid mutation in the Fc region, specifically an amino acid substitution. In a particular embodiment, the modification promoting association comprises a separate amino acid mutation, specifically one or more amino acid substitution, in each of the first Fc region and the second Fc region. See, e.g., § 5.3.2.2.

As used herein, the term “moiety” is used generically to describe any macro or micro molecule that can be operably connected to a polypeptide or protein described herein. Exemplary moieties include, but are not limited small molecules, polypeptides, polynucleotides (e.g., DNA, RNA), carbohydrates, lipids, synthetic polymers (e.g., polymers of PEG).

As used herein, the term “operably connected” refers to the linkage of two moieties (e.g., two polypeptides or two polynucleotides) in a functional relationship. For example, a polypeptide is operably connected to another polypeptide when they are linked (either directly or indirectly via a peptide linker) in frame such that both polypeptides are functional (e.g., a fusion protein or polypeptide described herein). Or for example, a transcription regulatory polynucleotide e.g., a promoter, enhancer, or other expression control element is operably linked to a polynucleotide that encodes a protein if it affects the transcription of the polynucleotide that encodes the protein. The term “operably connected” can also refer to the conjugation of a moiety to e.g., a polynucleotide or polypeptide (e.g., the conjugation of a PEG polymer to a protein or polypeptide).

The determination of “percent identity” between two sequences (e.g., peptide or protein (amino acid sequences) or polynucleotide (nucleic acid sequences)) can be accomplished using a mathematical algorithm. A specific, non-limiting example of a mathematical algorithm utilized for the comparison of two sequences is the algorithm of Karlin S & Altschul S F (1990) PNAS 87:2264-2268, modified as in Karlin S & Altschul S F (1993) PNAS 90:5873-5877, each of which is herein incorporated by reference in its entirety. Such an algorithm is incorporated into the NBLAST and XBLAST programs of Altschul S F et al., (1990) J Mol Biol 215:403, which is herein incorporated by reference in its entirety. BLAST nucleotide searches can be performed with the NBLAST nucleotide program parameters set, e.g., for score=100, wordlength=12 to obtain nucleotide sequences homologous to a nucleic acid molecule described herein. BLAST protein searches can be performed with the XBLAST program parameters set, e.g., to score 50, wordlength=3 to obtain amino acid sequences homologous to a protein molecule described herein. To obtain gapped alignments for comparison purposes, Gapped BLAST can be utilized as described in Altschul S F et al., (1997) Nuc Acids Res 25:3389-3402, which is herein incorporated by reference in its entirety. Alternatively, PSI BLAST can be used to perform an iterated search which detects distant relationships between molecules (Id.). When utilizing BLAST, Gapped BLAST, and PSI Blast programs, the default parameters of the respective programs (e.g., of XBLAST and NBLAST) can be used (see, e.g., National Center for Biotechnology Information (NCBI) on the worldwide web, ncbi.nlm.nih.gov). Another specific, non-limiting example of a mathematical algorithm utilized for the comparison of sequences is the algorithm of Myers and Miller, 1988, CABIOS 4:11-17, which is herein incorporated by reference in its entirety. Such an algorithm is incorporated in the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used. The percent identity between two sequences can be determined using techniques similar to those described above, with or without allowing gaps. In calculating percent identity, typically only exact matches are counted.

As used herein, the term “pharmaceutical composition” means a composition that is suitable for administration to an animal, e.g., a human subject, and comprises a therapeutic agent and a pharmaceutically acceptable carrier or diluent. A “pharmaceutically acceptable carrier or diluent” means a substance for use in contact with the tissues of human beings and/or non-human animals without excessive toxicity, irritation, allergic response, or other problem or complication, commensurate with a reasonable therapeutic benefit/risk ratio.

The terms “polynucleotide” and “nucleic acid molecule” are used interchangeably herein and refer to a polymer of DNA or RNA. The nucleic acid molecule can be single-stranded or double-stranded; contain natural, non-natural, or altered nucleotides; and contain a natural, non-natural, or altered internucleotide linkage, such as a phosphoroamidate linkage or a phosphorothioate linkage, instead of the phosphodiester found between the nucleotides of an unmodified nucleic acid molecule. Nucleic acid molecules include, but are not limited to, all nucleic acid molecules which are obtained by any means available in the art, including, without limitation, recombinant means, e.g., the cloning of nucleic acid molecules from a recombinant library or a cell genome, using ordinary cloning technology and polymerase chain reaction, and the like, and by synthetic means. The skilled artisan will appreciate that, except where otherwise noted, nucleic acid sequences set forth in the instant application will recite thymidine (T) in a representative DNA sequence but where the sequence represents RNA (e.g., mRNA), the thymidines (Ts) would be substituted for uracils (Us). Thus, any of the RNA polynucleotides encoded by a DNA identified by a particular sequence identification number may also comprise the corresponding RNA (e.g., mRNA) sequence encoded by the DNA, where each thymidine (T) of the DNA sequence is substituted with uracil (U).

As used herein, the term “polypeptide” refers to a polymer of at least 2 (e.g., at least 5) amino acids linked by a peptide bond. The term “polypeptide” does not denote a specific length of the polymer chain of amino acids. It is common in the art to refer to shorter polymers of amino acids (e.g., approximately 2-50 amino acids) as peptides; and to refer to longer polymers of amino acids (e.g., approximately over 50 amino acids) as polypeptides. However, the terms “peptide” and “polypeptide” are used interchangeably herein.

As used herein, the term “protein” refers to a polypeptide or a set (i.e., at least two) polypeptides. In embodiments where the protein comprises a set of polypeptides, the set of polypeptides associate to form a functional unit (i.e., quaternary structure). In some embodiments, the polypeptide or set of polypeptides are folded into their three-dimensional structure (i.e., tertiary or quaternary structure). Where polypeptides or sets of polypeptides are contemplated herein, it should be understood that proteins comprising the polypeptides or sets of polypeptides folded into their three-dimensional structure (i.e., tertiary or quaternary structure) are also provided herein and vice versa.

A “prophylactic” treatment is a treatment administered to a subject who does not exhibit signs of a disease or exhibits only early signs for the purpose of decreasing the risk of developing pathology.

The terms “RNA” and “polyribonucleotide” are used interchangeably herein and refer to macromolecules that include multiple ribonucleotides that are polymerized via phosphodiester bonds. Ribonucleotides are nucleotides in which the sugar is ribose. RNA may contain modified nucleotides; and contain natural, non-natural, or altered internucleotide linkages, such as a phosphoroamidate linkage or a phosphorothioate linkage, instead of the phosphodiester found between the nucleotides of an unmodified nucleic acid molecule.

As used herein, the term “sample” encompass a variety of biological specimens obtained from a subject. Exemplary sample types include, e.g., blood and other liquid samples of biological origin (including, but not limited to, whole-blood, peripheral blood mononuclear cells (PBMCs), serum, plasma, urine, saliva, amniotic fluid, stool, synovial fluid, etc.), nasopharyngeal swabs, solid tissue samples such as biopsies (or cells derived therefrom and the progeny thereof), tissue cultures (or cells derived therefrom and the progeny thereof), and cell cultures (or cells derived therefrom and the progeny thereof). The term also includes samples that have been manipulated in any way after their procurement from a subject, such as by centrifugation, filtration, washing, precipitation, dialysis, chromatography, lysis, treatment with reagents, enriched for certain cell populations, refrigeration, freezing, staining, etc.

The term “scFv” or “single chain variable fragment” refers to an antibody that comprises a VH region operably connected via a peptide linker to a VL region, wherein the VH and VL regions associate to specifically bind an antigen (e.g., form an antigen binding domain). In some embodiments, the scFv comprises from N- to C-terminus an VH region, a peptide linker, and an VL region. In some embodiments, the scFv comprises from N- to C-terminus an VL region, a peptide linker, and an VH region.

The term “(scFv)₂” as used herein refers to an antibody that comprises a first scFv operably connected (e.g., via a peptide linker) to a second scFv. The first and second scFv can specifically bind the same or different antigens. In some embodiments, the first and second scFv are operably connected via a peptide linker.

The term “scFv-Fc” as used herein refers to an antibody that comprises a scFv operably linked (e.g., via a peptide linker) to an Fc region. In some embodiments, a scFv is operably connected to only a first Fc region of a protein comprising a first and a second Fc region. In some embodiments, a first scFv is operably connected to a first Fc region and a second scFv is operably connected to a second Fc region of a protein comprising a first and a second Fc region.

The term “(scFv)₂-Fc” as used herein refers to a (scFv)₂ operably linked (e.g., via a peptide linker) to an Fc region. In some embodiments, a (scFv)₂ is operably connected to only a first Fc region of a protein comprising a first and a second Fc region. In some embodiments, a first (scFv)₂ is operably connected to a first Fc region and a second (scFv)₂ is operably connected to a second Fc region of a protein comprising a first and a second Fc region.

As used herein, the term “single domain antibody” or “sdAb” refers to an antibody having a single monomeric variable antibody domain. A sdAb is able to specifically bind to a specific antigen. A VHH (as defined herein) is an example of a sdAb.

As used herein, the term “signal peptide” or “signal sequence” refers to a sequence (e.g., an amino acid sequence) that can direct the transport or localization of a protein to a certain organelle, cell compartment, or extracellular export. The term encompasses both the signal sequence peptide and the nucleic acid sequence encoding the signal peptide. Thus, references to a signal peptide in the context of a nucleic acid refers to the nucleic acid sequence encoding the signal peptide.

As used herein, the term “specifically binds” refers to the preferential interaction, i.e., significantly higher binding affinity, between a first protein (e.g., a ligand) and a second protein (e.g., the ligand's cognate receptor) relative to other amino acid sequences. Herein, when a first protein or polypeptide is said to “specifically bind” to a second protein or polypeptide, it is understood that the first protein or polypeptide specifically binds to an epitope of the second protein or polypeptide. The term “epitope” refers to the portion of the second protein or polypeptide that the first protein or polypeptide specifically recognizes. The term specifically binds includes molecules that are cross reactive with the same epitope of a different species. For example, an antibody that specifically binds human CAIX may be cross reactive with CAIX of another species (e.g., cynomolgus, murine, etc.), and still be considered herein to specifically bind human CAIX.

As used herein, the term “subject” includes any animal, such as a human or other animal. In some embodiments, the subject is a vertebrate animal (e.g., mammal, bird, fish, reptile, or amphibian). In some embodiments, the subject is a human. In some embodiments, the method subject is a non-human mammal. In some embodiments, the subject is a non-human mammal is such as a non-human primate (e.g., monkeys, apes), ungulate (e.g., cattle, buffalo, sheep, goat, pig, camel, llama, alpaca, deer, horses, donkeys), carnivore (e.g., dog, cat), rodent (e.g., rat, mouse), or lagomorph (e.g., rabbit). In some embodiments, the subject is a bird, such as a member of the avian taxa Galliformes (e.g., chickens, turkeys, pheasants, quail), Anseriformes (e.g., ducks, geese), Paleaognathae (e.g., ostriches, emus), Columbiformes (e.g., pigeons, doves), or Psittaciformes (e.g., parrots).

As used herein, the term “therapeutically effective amount” of a therapeutic agent refers to any amount of the therapeutic agent that, when used alone or in combination with another therapeutic agent, protects a subject against the onset of a disease or promotes disease regression evidenced by a decrease in severity of disease of infection symptoms, an increase in frequency and duration of disease or infection symptom-free periods, or a prevention of impairment or disability due to the disease or infection affliction. The ability of a therapeutic agent to promote disease regression can be evaluated using a variety of methods known to the skilled practitioner, such as in human subjects during clinical trials, in animal model systems predictive of efficacy in humans, or by assaying the activity of the agent in in vitro assays.

As used herein, the terms “treat,” treating,” “treatment,” and the like refer to reducing or ameliorating a disease and/or symptom(s) associated therewith or obtaining a desired pharmacologic and/or physiologic effect. It will be appreciated that, although not precluded, treating a disease does not require that the disease, or symptom(s) associated therewith be completely eliminated. In some embodiments, the effect is therapeutic, i.e., without limitation, the effect partially or completely reduces, diminishes, abrogates, abates, alleviates, decreases the intensity of, or cures a disease and/or adverse symptom attributable to the disease. In some embodiments, the effect is preventative, i.e., the effect protects or prevents an occurrence or reoccurrence of a disease. To this end, the presently disclosed methods comprise administering a therapeutically effective amount of a compositions as described herein.

As used herein, the term “variable region” refers to a portion of an antibody, generally, a portion of a light or heavy chain, typically about the amino-terminal 110 to 120 amino acids or 110 to 125 amino acids in the mature heavy chain and about 90 to 115 amino acids in the mature light chain, which differ extensively in sequence among antibodies and are used in the binding and specificity of a particular antibody for its particular antigen. The variability in sequence is concentrated in those regions called complementarity determining regions (CDRs) while the more highly conserved regions in the variable domain are called framework regions (FR). Without wishing to be bound by any particular mechanism or theory, it is believed that the CDRs of the light and heavy chains are primarily responsible for the interaction and specificity of the antibody with antigen. In certain embodiments, the variable region is a human variable region. In certain embodiments, the variable region comprises rodent or murine CDRs and human framework regions (FRs). In particular embodiments, the variable region is a primate (e.g., non-human primate) variable region. In certain embodiments, the variable region comprises rodent or murine CDRs and primate (e.g., non-human primate) framework regions (FRs).

The terms “VL” and “VL region” are used interchangeably to refer to an immunoglobulin light chain variable region. A VL region can be incorporated into an antibody, e.g., a scFv, a Fab, a full-length antibody. For example, a scFv comprises a VL region operably connected via a peptide linker to a VH region.

The terms “VH” and “VH region” are used interchangeably to refer to an immunoglobulin heavy chain variable region. A VH region can be incorporated into an antibody, e.g., a scFv, a Fab, a full-length antibody. For example, a scFv comprises a VH region operably connected via a peptide linker to a VL region.

The term “VHH” as used herein refers to a type of single domain antibody (sdAb) that has a single monomeric heavy chain variable antibody domain (VH). Such antibodies can be found in or produced from camelid mammals (e.g., camels, llamas) which are naturally devoid of light chains or synthetically produced.

The term “(VHH)₂” as used herein refers to an antibody that comprises a first VHH operably connected to a second VHH (e.g., via a peptide linker). The first and the second VHH can specifically bind the same or different antigens. In some embodiments, the first and second VHH are operably connected by a peptide linker.

The term “VHH-Fc” as used herein refers to an antibody that comprises a VHH operably linked (e.g., via a peptide linker) to an Fc region. In some embodiments, a VHH is operably connected to only a first Fc region of a protein that comprises a first Fc region and a second Fc region. In some embodiments, a first VHH is operably connected to a first Fc region and a second VHH is operably connected to a second Fc region of a protein comprising a first and a second Fc region.

The term “(VHH)₂-Fc” as used herein refers to (VHH)₂ operably linked (e.g., via a peptide linker) to an Fc region. In some embodiments, a (VHH)₂ is operably connected to only a first Fc region of a protein comprising a first and a second Fc region. In some embodiments, a first (VHH)₂ is operably connected to a first region and a second (VHH)₂ is operably connected to a second Fc domain a protein comprising a first and a second Fc region.

### 5.1.1 Humanized Anti-CAIX Antibodies

Human CAIX (hCAIX) is a transmembrane dimeric metalloenzyme with an extracellular active site that facilitates acid secretion in the gastrointestinal tract and is one of the 14 carbonic anhydrase isoforms found in humans. The amino acid sequence of an exemplary mature (SEQ ID NO: 1) and immature (SEQ ID NO: 2) reference hCAIX polypeptide is provided in Table 1. The N-terminal amino acids 1-37 of SEQ ID NO: 2 (underlined) represent the signal peptide.

TABLE 1
The Amino Acid Sequence of Exemplary hCAIX Polypeptide

| Description | SEQ ID NO | Amino Acid Sequence |
|---|---|---|
| hCAIX | 1 | QRLPRMQEDSPLGGGSSGEDDPLGEEDLPSEEDSPREEDPPG |
| Mature (No signal | | EEDLPGEEDLPGEEDLPEVKPKSEEEGSLKLEDLPTVEAPGD |
| sequence) | | PQEPQNNAHRDKEGDDQSHWRYGGDPPWPRVSPACAGRFQSP |
| Extracellular | | VDIRPQLAAFCPALRPLELLGFQLPPLPELRLRNNGHSVQLT |
| domain bold | | LPPGLEMALGPGREYRALQLHLHWGAAGRPGSEHTVEGHRFP |
| UniProt ID: Q16790 | | AEIHVVHLSTAFARVDEALGRPGGLAVLAAFLEEGPEENSAY |
| | | EQLLSRLEEIAEEGSETQVPGLDISALLPSDFSRYFQYEGSL |
| | | TTPPCAQGVIWTVFNQTVMLSAKQLHTLSDTLWGPGDSRLQL |
| | | NFRATQPLNGRVIEASFPAGVDSSPRAAEPVQLNSCLAAGDI |
| | | LALVFGLLFAVTSVAFLVQMRRQHRRGTKGGVSYRPAEVAET |
| | | GA |
| hCAIX | 2 | MAPLCPSPWLPLLIPAPAPGLTVQLLLSLLLLVPVHPQRLPR |
| Immature (Signal | | MQEDSPLGGGSSGEDDPLGEEDLPSEEDSPREEDPPGEEDLP |
| sequence Underlined) | | GEEDLPGEEDLPEVKPKSEEEGSLKLEDLPTVEAPGDPQEPQ |
| Extracellular | | NNAHRDKEGDDQSHWRYGGDPPWPRVSPACAGRFQSPVDIRP |
| domain bold | | QLAAFCPALRPLELLGFQLPPLPELRLRNNGHSVQLTLPPGL |
| UniProt ID: Q16790 | | EMALGPGREYRALQLHLHWGAAGRPGSEHTVEGHRFPAEIHV |
| | | VHLSTAFARVDEALGRPGGLAVLAAFLEEGPEENSAYEQLLS |
| | | RLEEIAEEGSETQVPGLDISALLPSDFSRYFQYEGSLTTPPC |
| | | AQGVIWTVFNQTVMLSAKQLHTLSDTLWGPGDSRLQLNFRAT |
| | | QPLNGRVIEASFPAGVDSSPRAAEPVQLNSCLAAGDILALVF |
| | | GLLFAVTSVAFLVQMRRQHRRGTKGGVSYRPAEVAETGA |

In one aspect, provided herein are antibodies (and functional fragments and variants thereof (e.g., antigen binding domains thereof)) that specifically bind hCAIX, such antibodies are also referred to herein as anti-CAIX antibodies.

The amino acid sequence of the VH and VL regions of exemplary anti-hCAIX antibodies is provided in Table 2.

TABLE 2
The Amino Acid Sequence of Exemplary Anti-hCAIX VH and VL Polypeptides

| | | |
|---|---|---|
| | | SEQ ID |
| Description | Amino Acid Sequence | NO |

VH

| | | |
|---|---|---|
| HC1 | EVQLLESGGGLVQPGGSLKLSCAASGFTFSNYYMSWVRQAPGKGLEWVSA | 3 |
| | INSDGGITYYLDTVKGRFTISRDNSKNTLYLQMNSLRAEDTAVYYCARHR | |
| | SGYFSMDYWGQGTTVTVSS | |
| HC2 | LVQLVESGGGLVKPGGSLRLSCAASGFTFSNYYMSWIRQAPGKGLEWVSA | 4 |
| | INSDGGITYYLDTVKGRFTISRDNAKNSLYLQMNSLRAEDTAVYYCARHR | |
| | SGYFSMDYWGQGTLVTVSS | |
| HC3 | EVQLVESGGGLVQPGGSLRLSCAASGFTFSNYYMSWVRQAPGKGLEWVAA | 5 |
| | INSDGGITYYLDTVKGRFTISRDNAKNSLYLQMNSLRAEDTAVYYCARHR | |
| | SGYFSMDYWGQGTLVTVSS | |
| HC4 | EVQLLESGGGLVQPGGSLRLSCAASGFTFSNYYMSWVRQAPGKGLEWVSAINSDG | 6 |
| | GITYYLDTVKGRFTISRDDSKNTLYLQMSSLRAEDTAVYYCARHRSGYFSMDYWG | |
| | QGTTVTVSS | |
| HC5 | EVRLVESGGGLVKPGGSLRLSCAASGFTFSNYYMSWIRQAPEKRLELVSA | 7 |
| | INSDGGITYYLDTVKGRFTISRDNAKNSLYLQMNSLRAEDTALFYCARHR | |
| | SGYFSMDYWGQGTSVTVSS | |
| HC5 | EVRLVESGGGLVKPGGSLRLSCAASGFTFSNYYMSWIRQAPEKRLELVSA | 7 |
| Variant 3 | INSDGGITYYLDTVKGRFTISRDNAKNSLYLQMNSLRAEDTALFYCARHR | |
| | SGYFSMDYWGQGTSVTVSS | |
| HC5 | EVRLVESGGGLVKPGGSLRLSCAASGFTFSNYYMSWIRQAPEKRLELVSA | 8 |
| Variant 27 | INSDGAITYYLDTVKGRFTISRDNAKNSLYLQMNSLRAEDTALFYCARHR | |
| | SGYFSMDYWGQGTSVTVSS | |
| HC5 | EVRLVESGGGLVKPGGSLRLSCAVSGFTFSNYYMSWIRQAPEKRLELVSA | 9 |
| Variant 29 | INSDGGITYYLDTVKGRFTISRDNAKNSLYLQMNSLRAEDTALFYCARHR | |
| | SGYFSMDYWGQGTSVTVSS | |
| HC5 | EVRLVESGGGLVKPGGSLRLSCAVSGFTFSNYYMSWIRQAPEKRLELVSA | 9 |
| Variant 36 | INSDGGITYYLDTVKGRFTISRDNAKNSLYLQMNSLRAEDTALFYCARHR | |
| | SGYFSMDYWGQGTSVTVSS | |
| HC5 | EVRLVESGGGLVKPGGSLRLSCAASGFTFSNYYMSWIRQAPEKRLELVSA | 8 |
| Variant 74 | INSDGAITYYLDTVKGRFTISRDNAKNSLYLQMNSLRAEDTALFYCARHR | |
| | SGYFSMDYWGQGTSVTVSS | |

VL

| | | |
|---|---|---|
| LC1 | DIVMTQSPATLSLSPGDRATLSCKASQNVVSAVAWYQQKPGQAPRLLIYS | 10 |
| | ASNRYTGIPARFSGSGSGTDETLTISSLQSEDFAVYYCQQYSNYPWTFGG | |
| | GTKVEIK | |
| LC2 | EIVMTQSPATLSVSPGERATLSCKASQNVVSAVAWYQQKPGQAPRLLVYS | 11 |
| | ASNRYTGIPDRESGSGSGTEFTLTISSLQSEDFAVYYCQQYSNYPWTFGQ | |
| | GTKLEIK | |
| LC3 | EIVMTQSPATLSVSPGERATLSCKASQNVVSAVAWYQQKPGQSPRLLIYS | 12 |
| | ASNRYTGIPARFSGSGSGTEFTLTISSLQSEDFAAYYCQQYSNYPWTFGG | |
| | GTKVEIK | |
| LC4 | DIQMTQSPFSLSASVGDRVTITCKASQNVVSAVAWYQQKPGKAPKLLIYS | 13 |
| | ASNRYTGVPSRFSGSGSGTDFTLTISSLQPEDFATYFCQQYSNYPWTEGG | |
| | GTKLEIK | |
| LC5 | AIQLTQSQRFLSASVGDRVTITCRASQNVVSALAWYQQKPGQSPKLLIYS | 14 |
| | ASNRYTGVPSRFSGSGSGTDFTLTISSLQPEDFADFFCQQYSNYPWTFGG | |
| | GTKLEIK | |
| LC3 | EIVMTQSPATLSVSPGERATLSCKASQNVVSAVAWYQQKPGQSPRLLIYS | 15 |
| Variant 3 | ASNRYTGIPARFSGSGSGTEFTLTISSLQSEDFAAYYCQQYRNYPWTFGG | |
| | GTKVEIK | |
| LC3 | EIVMTQSPATLSVSPGERATLSCKASQNVVSAVAWYQQKPGQSPRLLIYS | 15 |
| Variant 27 | ASNRYTGIPARFSGSGSGTEFTLTISSLQSEDFAAYYCQQYRNYPWTFGG | |
| | GTKVEIK | |
| LC3 | EIVMTQSPATLSVSPGERATLSCKASQNVVSAVAWYQQKPGQSPRLLIYS | 15 |
| Variant 29 | ASNRYTGIPARFSGSGSGTEFTLTISSLQSEDFAAYYCQQYRNYPWTFGG | |
| | GTKVEIK | |
| LC3 | EIVMTQSPATLSVSPGERATLSCKASQNVVSAVGWYQQKPGQSPRLLIYS | 16 |
| Variant 36 | ASNRYTGIPARFSGSGSGTEFTLTISSLQSEDFAAYYCQQYSNYPWTFGG | |
| | GTKVEIK | |
| LC3 | EIVMTQSPATLSVSPGERVTLSCKASQNVVSAVGWYQKKPGQSPRLLIYS | 17 |
| Variant 74 | ASNRYTGIPARFSGSGSGTEFTLTISSLQSEDFAAYYCQQYNNYPWTFGG | |
| | GTKVEIK | |

In some embodiments, the anti-hCAIX antibody (or functional fragment or variant thereof) comprises a VH and a VL.

In some embodiments, the amino acid sequence of the VH is at least 85%, 86%, 87%, 88%, 89%, 90%, 91%, 92%, 93%, 94%, 95%, 96%, 97%, 98%, 99%, or 100% identical to the amino acid sequence of any VH polypeptide set forth in Table 2; and the amino acid sequence of the VL is at least 85%, 86%, 87%, 88%, 89%, 90%, 91%, 92%, 93%, 94%, 95%, 96%, 97%, 98%, 99%, or 100% identical to the amino acid sequence of any VL polypeptide set forth in Table 2.

In some embodiments, the amino acid sequence of the VH comprises the amino acid sequence of any VH polypeptide set forth in Table 2; and the amino acid sequence of the VL comprises the amino acid sequence of any VL polypeptide set forth in Table 2.

In some embodiments, the amino acid sequence of the VH is at least 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 3-9; and the amino acid sequence of the VL is at least 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 10-17.

In some embodiments, the amino acid sequence of the VH is at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 3; and the amino acid sequence of the VL is at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 10. In some embodiments, the amino acid sequence of the VH is at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 3; and the amino acid sequence of the VL is at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 11. In some embodiments, the amino acid sequence of the VH is at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 3; and the amino acid sequence of the VL is at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 12. In some embodiments, the amino acid sequence of the VH is at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 3; and the amino acid sequence of the VL is at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 13. In some embodiments, the amino acid sequence of the VH is at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 3; and the amino acid sequence of the VL is at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 14. In some embodiments, the amino acid sequence of the VH is at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 3; and the amino acid sequence of the VL is at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 15. In some embodiments, the amino acid sequence of the VH is at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 3; and the amino acid sequence of the VL is at least 85%, 86%, 87%, 88%, 89%, 90%, 91%, 92%, 93%, 94%, 95%, 96%, 97%, 98%, 99%, or 100% identical to the amino acid sequence set forth in SEQ ID NO:16. In some embodiments, the amino acid sequence of the VH is at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 3; and the amino acid sequence of the VL is at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 17.

In some embodiments, the amino acid sequence of the VH is at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 4; and the amino acid sequence of the VL is at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 10. In some embodiments, the amino acid sequence of the VH is at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 4; and the amino acid sequence of the VL is at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 11. In some embodiments, the amino acid sequence of the VH is at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 4; and the amino acid sequence of the VL is at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 12. In some embodiments, the amino acid sequence of the VH is at least 85%, 86%, 87%, 88%, 89%, 90%, 91%, 92%, 93%, 94%, 95%, 96%, 97%, 98%, 99%, or 100% identical to the amino acid sequence set forth SEQ ID NO: 4; and the amino acid sequence of the VL is at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 13. In some embodiments, the amino acid sequence of the VH is at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 4; and the amino acid sequence of the VL is at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 14. In some embodiments, the amino acid sequence of the VH is at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 4; and the amino acid sequence of the VL is at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 15. In some embodiments, the amino acid sequence of the VH is at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 4; and the amino acid sequence of the VL is at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 16. In some embodiments, the amino acid sequence of the VH is at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 4; and the amino acid sequence of the VL is at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 17.

In some embodiments, the amino acid sequence of the VH is at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 5; and the amino acid sequence of the VL is at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 10. In some embodiments, the amino acid sequence of the VH is at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 5; and the amino acid sequence of the VL is at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 11. In some embodiments, the amino acid sequence of the VH is at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 5; and the amino acid sequence of the VL is at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 12. In some embodiments, the amino acid sequence of the VH is at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 5; and the amino acid sequence of the VL is at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 13. In some embodiments, the amino acid sequence of the VH is at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 5; and the amino acid sequence of the VL is at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 14. In some embodiments, the amino acid sequence of the VH is at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 5; and the amino acid sequence of the VL is at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 15. In some embodiments, the amino acid sequence of the VH is at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 5; and the amino acid sequence of the VL is at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 16. In some embodiments, the amino acid sequence of the VH is at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 5; and the amino acid sequence of the VL is at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 17.

In some embodiments, the amino acid sequence of the VH is at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 6; and the amino acid sequence of the VL is at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 10. In some embodiments, the amino acid sequence of the VH is at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 6; and the amino acid sequence of the VL is at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 11. In some embodiments, the amino acid sequence of the VH is at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 6; and the amino acid sequence of the VL is at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 12. In some embodiments, the amino acid sequence of the VH is at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 6; and the amino acid sequence of the VL is at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 13. In some embodiments, the amino acid sequence of the VH is at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 6; and the amino acid sequence of the VL is at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 14. In some embodiments, the amino acid sequence of the VH is at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 6; and the amino acid sequence of the VL is at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 15. In some embodiments, the amino acid sequence of the VH is at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 6; and the amino acid sequence of the VL is at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 16. In some embodiments, the amino acid sequence of the VH is at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 6; and the amino acid sequence of the VL is at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 17.

In some embodiments, the amino acid sequence of the VH is at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 7; and the amino acid sequence of the VL is at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 10. In some embodiments, the amino acid sequence of the VH is at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 7; and the amino acid sequence of the VL is at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 11. In some embodiments, the amino acid sequence of the VH is at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 7; and the amino acid sequence of the VL is at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 12. In some embodiments, the amino acid sequence of the VH is at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 7; and the amino acid sequence of the VL is at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 13. In some embodiments, the amino acid sequence of the VH is at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 7; and the amino acid sequence of the VL is at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 14. In some embodiments, the amino acid sequence of the VH is at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 7; and the amino acid sequence of the VL is at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 15. In some embodiments, the amino acid sequence of the VH is at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 7; and the amino acid sequence of the VL is at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 16. In some embodiments, the amino acid sequence of the VH is at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 7; and the amino acid sequence of the VL is at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 17.

In some embodiments, the amino acid sequence of the VH is at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 8; and the amino acid sequence of the VL is at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 10. In some embodiments, the amino acid sequence of the VH is at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 8; and the amino acid sequence of the VL is at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 11. In some embodiments, the amino acid sequence of the VH is at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 8; and the amino acid sequence of the VL is at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 12. In some embodiments, the amino acid sequence of the VH is at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 8; and the amino acid sequence of the VL is at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 13. In some embodiments, the amino acid sequence of the VH is at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 8; and the amino acid sequence of the VL is at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 14. In some embodiments, the amino acid sequence of the VH is at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 8; and the amino acid sequence of the VL is at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 15. In some embodiments, the amino acid sequence of the VH is at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 8; and the amino acid sequence of the VL is at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 16. In some embodiments, the amino acid sequence of the VH is at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 8; and the amino acid sequence of the VL is at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 17.

In some embodiments, the amino acid sequence of the VH is at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 9; and the amino acid sequence of the VL is at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 10. In some embodiments, the amino acid sequence of the VH is at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 9; and the amino acid sequence of the VL is at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 11. In some embodiments, the amino acid sequence of the VH is at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 9; and the amino acid sequence of the VL is at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 12. In some embodiments, the amino acid sequence of the VH is at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 9; and the amino acid sequence of the VL is at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 13. In some embodiments, the amino acid sequence of the VH is at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 9; and the amino acid sequence of the VL is at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 14. In some embodiments, the amino acid sequence of the VH is at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 9; and the amino acid sequence of the VL is at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 15. In some embodiments, the amino acid sequence of the VH is at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 9; and the amino acid sequence of the VL is at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 16. In some embodiments, the amino acid sequence of the VH is at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 9; and the amino acid sequence of the VL is at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 17.

The nucleotide sequence of the VH and VL regions of the exemplary anti-hCAIX antibodies is provided in Table 3.

TABLE 3
The Nucleotide Sequence of Exemplary Anti-CAIX VH and VL Polypeptides

| | | |
|---|---|---|
| | | SEQ ID |
| Description | Nucleotide Sequence | NO |

VH

| | | |
|---|---|---|
| HC1 | GAGGTGCAGCTGCTGGAATCTGGAGGAGGCCTTGTGCAACCTGGAGGCAG | 18 |
| | CCTGAAGCTGAGCTGTGCTGCCTCTGGCTTCACCTTCAGCAACTACTACA | |
| | TGAGCTGGGTGAGACAAGCCCCTGGCAAGGGCCTGGAGTGGGTGTCTGCC | |
| | ATCAACTCTGATGGAGGCATCACCTACTACCTGGACACAGTGAAGGGCAG | |
| | ATTCACCATCAGCAGAGATAATAGCAAAAACACCCTGTACCTGCAGATGA | |
| | ACAGCCTGAGAGCTGAGGACACAGCTGTGTACTACTGTGCTAGACACAGA | |
| | TCTGGCTACTTCAGCATGGACTACTGGGGCCAAGGCACCACAGTGACTGT | |
| | GAGCTCT | |
| HC2 | CTGGTGCAACTGGTAGAATCTGGAGGAGGCCTTGTGAAACCTGGAGGCAG | 19 |
| | CCTGAGACTGAGCTGTGCTGCCTCTGGCTTCACCTTCAGCAACTACTACA | |
| | TGAGCTGGATCAGACAAGCCCCTGGCAAGGGCCTGGAGTGGGTGTCTGCC | |
| | ATCAACTCTGATGGAGGCATCACCTACTACCTGGACACAGTGAAGGGCAG | |
| | ATTCACCATCAGCAGAGATAATGCCAAAAACAGCCTGTACCTGCAGATGA | |
| | ACAGCCTGAGAGCTGAGGACACAGCTGTGTACTACTGTGCTAGACACAGA | |
| | TCTGGCTACTTCAGCATGGACTACTGGGGCCAAGGCACCCTGGTGACTGT | |
| | GAGCTCT | |
| HC3 | GAGGTGCAACTGGTAGAATCTGGAGGAGGCCTTGTGCAGCCTGGAGGCAG | 20 |
| | CCTGAGACTGAGCTGTGCTGCCTCTGGCTTCACCTTCAGCAACTACTACA | |
| | TGAGCTGGGTGAGACAAGCCCCTGGCAAGGGCCTGGAGTGGGTGGCTGCC | |
| | ATCAACTCTGATGGAGGCATCACCTACTACCTGGACACAGTGAAGGGCAG | |
| | ATTCACCATCAGCAGAGATAATGCCAAAAACAGCCTGTACCTGCAGATGA | |
| | ACAGCCTGAGAGCTGAGGACACAGCTGTGTACTACTGTGCTAGACACAGA | |
| | TCTGGCTACTTCAGCATGGACTACTGGGGCCAAGGCACCCTGGTGACTGT | |
| | GAGCTCT | |
| HC4 | GAGGTGCAGCTGCTGGAATCTGGAGGAGGCCTTGTGCAACCTGGAGGCAG | 21 |
| | CCTGAGACTGAGCTGTGCTGCCTCTGGCTTCACCTTCAGCAACTACTACA | |
| | TGAGCTGGGTGAGACAAGCCCCTGGCAAGGGCCTGGAGTGGGTGTCTGCC | |
| | ATCAACTCTGATGGAGGCATCACCTACTACCTGGACACAGTGAAGGGCAG | |
| | ATTCACCATCAGCAGAGATGACAGCAAAAACACCCTGTACCTGCAGATGA | |
| | GCAGCCTGAGAGCTGAGGACACAGCTGTGTACTACTGTGCTAGACACAGA | |
| | TCTGGCTACTTCAGCATGGACTACTGGGGCCAAGGCACCACAGTGACTGT | |
| | GAGCTCT | |
| HC5 | GAGGTCAGACTGGTAGAATCTGGAGGAGGCCTTGTGAAACCTGGAGGCAG | 22 |
| | CCTGAGACTGAGCTGTGCTGCCTCTGGCTTCACCTTCAGCAACTACTACA | |
| | TGAGCTGGATCAGACAAGCCCCTGAGAAGAGACTGGAGCTGGTGTCTGCC | |
| | ATCAACTCTGATGGAGGCATCACCTACTACCTGGACACAGTGAAGGGCAG | |
| | ATTCACCATCAGCAGAGATAATGCCAAAAACAGCCTGTACCTGCAGATGA | |
| | ACAGCCTGAGAGCTGAGGACACAGCCCTGTTCTACTGTGCTAGACACAGA | |
| | TCTGGCTACTTCAGCATGGACTACTGGGGCCAAGGCACCTCTGTGACTGT | |
| | GAGCTCT | |
| HC5 | GAAGTCCGTCTGGTCGAGAGCGGGGGGGGCCTGGTGAAGCCTGGCGGCAG | 23 |
| Codon | CCTGAGGCTGTCTTGCGCCGCTTCCGGCTTCACCTTTTCTAACTACTATA | |
| Optimized | TGTCCTGGATCAGACAGGCCCCAGAGAAGAGGCTGGAGCTGGTGTCCGCC | |
| | ATCAACTCTGACGGCGGCATCACATACTATCTGGATACCGTGAAGGGCAG | |
| | ATTCACAATCTCTCGCGATAACGCCAAGAACTCCCTGTACCTCCAGATGA | |
| | ACAGCCTGCGGGCCGAGGACACCGCTCTGTTCTATTGCGCTAGGCACCGG | |
| | TCCGGCTACTTTAGCATGGATTATTGGGGCCAGGGCACCTCCGTCACAGT | |
| | GTCCAGC | |

VL

| | | |
|---|---|---|
| LC1 | GACATTGTGATGACACAGTCTCCTGCCACCCTGAGCCTGAGCCCTGGAGA | 24 |
| | CAGAGCTACCCTGAGCTGCAAGGCTTCTCAGAATGTGGTGTCTGCTGTGG | |
| | CCTGGTATCAGCAGAAGCCTGGACAAGCCCCTAGACTGCTGATCTACTCT | |
| | GCTAGCAACAGATACACTGGAATCCCTGCTAGATTCTCTGGCTCTGGCTC | |
| | TGGCACAGACTTCACCCTGACCATCAGCAGCCTGCAGTCTGAGGACTTTG | |
| | CTGTGTACTACTGTCAGCAGTACAGCAACTACCCTTGGACCTTTGGAGGA | |
| | GGCACCAAGGTGGAGATCAAG | |
| LC2 | GAGATTGTGATGACACAGTCTCCTGCCACCCTGTCTGTGAGCCCTGGAGA | 25 |
| | GAGAGCTACCCTGAGCTGCAAGGCTTCTCAGAATGTGGTGTCTGCTGTGG | |
| | CCTGGTATCAGCAGAAGCCTGGACAAGCCCCTAGACTGCTGGTGTACTCT | |
| | GCTAGCAACAGATACACTGGAATCCCTGACAGATTCTCTGGCTCTGGCTC | |
| | TGGCACAGAGTTCACCCTGACCATCAGCAGCCTGCAGTCTGAGGACTTTG | |
| | CTGTGTACTACTGTCAGCAGTACAGCAACTACCCTTGGACCTTTGGACAA | |
| | GGCACCAAGCTGGAGATCAAG | |
| LC3 | GAGATTGTGATGACACAGTCTCCTGCCACCCTGTCTGTGAGCCCTGGAGA | 26 |
| | GAGAGCTACCCTGAGCTGCAAGGCTTCTCAGAATGTGGTGTCTGCTGTGG | |
| | CCTGGTATCAGCAGAAGCCTGGACAGAGCCCTAGACTGCTGATCTACTCT | |
| | GCTAGCAACAGATACACTGGAATCCCTGCTAGATTCTCTGGCTCTGGCTC | |
| | TGGCACAGAGTTCACCCTGACCATCAGCAGCCTGCAGTCTGAGGACTTTG | |
| | CTGCCTACTACTGTCAGCAGTACAGCAACTACCCTTGGACCTTTGGAGGA | |
| | GGCACCAAGGTGGAGATCAAG | |
| LC3 | GAAATTGTGATGACTCAGAGCCCCGCAACTCTGAGCGTGTCTCCCGGCGA | 27 |
| Codon | GAGAGCCACCCTGTCTTGCAAGGCTTCCCAGAACGTGGTGTCTGCCGTGG | |
| Optimized | CTTGGTATCAGCAGAAGCCAGGCCAGTCCCCAAGGCTGCTGATCTACTCC | |
| | GCCAGCAATAGGTATACCGGCATCCCTGCTCGGTTCTCTGGCTCCGGCAG | |
| | CGGCACAGAGTTTACCCTGACCATCTCCTCCCTCCAGAGCGAGGACTTCG | |
| | CCGCTTACTATTGCCAGCAGTACTCTAACTATCCTTGGACCTTTGGCGGC | |
| | GGCACAAAGGTGGAGATCAAG | |
| LC4 | GACATTCAGATGACACAGTCTCCTTTCAGCCTGTCTGCCTCTGTGGGAGA | 28 |
| | CAGAGTGACCATCACCTGCAAGGCTTCTCAGAATGTGGTGTCTGCTGTGG | |
| | CCTGGTATCAGCAGAAGCCTGGAAAGGCCCCTAAGCTGCTGATCTACTCT | |
| | GCTAGCAACAGATACACTGGAGTGCCTAGCAGATTCTCTGGCTCTGGCTC | |
| | TGGCACAGACTTCACCCTGACCATCAGCAGCCTGCAGCCTGAGGACTTTG | |
| | CCACCTACTTCTGTCAGCAGTACAGCAACTACCCTTGGACCTTTGGAGGA | |
| | GGCACCAAGCTGGAGATCAAG | |
| LC5 | GCCATTCAGCTGACACAGTCTCAGAGATTCCTGTCTGCCTCTGTGGGAGA | 29 |
| | CAGAGTGACCATCACCTGCAGAGCTTCTCAGAATGTGGTGTCTGCTCTGG | |
| | CCTGGTATCAGCAGAAGCCTGGACAGAGCCCTAAGCTGCTGATCTACTCT | |
| | GCTAGCAACAGATACACTGGAGTGCCTAGCAGATTCTCTGGCTCTGGCTC | |
| | TGGCACAGACTTCACCCTGACCATCAGCAGCCTGCAGCCTGAGGACTTTG | |
| | CTGACTTCTTCTGTCAGCAGTACAGCAACTACCCTTGGACCTTTGGAGGA | |
| | GGCACCAAGCTGGAGATCAAG | |

In some embodiments, the nucleotide sequence of the VH comprises a nucleotide sequence at least 85%, 86%, 87%, 88%, 89%, 90%, 91%, 92%, 93%, 94%, 95%, 96%, 97%, 98%, 99%, or 100% identical to the nucleotide sequence of any VH set forth in Table 3; and the nucleotide sequence of the VL comprises a nucleotide sequence at least 85%, 86%, 87%, 88%, 89%, 90%, 91%, 92%, 93%, 94%, 95%, 96%, 97%, 98%, 99%, or 100% identical to the nucleotide sequence of any VL set forth in Table 3.

In some embodiments, the nucleotide sequence of the VH is at least 85%, 86%, 87%, 88%, 89%, 90%, 91%, 92%, 93%, 94%, 95%, 96%, 97%, 98%, 99%, or 100% identical to the nucleotide sequence set forth in any one of SEQ ID NOS: 18-23; and the nucleotide sequence of the VH is at least 85%, 86%, 87%, 88%, 89%, 90%, 91%, 92%, 93%, 94%, 95%, 96%, 97%, 98%, 99%, or 100% identical to the nucleotide sequence set forth in any one of SEQ ID NOS: 24-29.

In some embodiments, the nucleotide sequence of the VH is at least 85%, 86%, 87%, 88%, 89%, 90%, 91%, 92%, 93%, 94%, 95%, 96%, 97%, 98%, 99%, or 100% identical to the nucleotide sequence set forth in SEQ ID NO: 23; and the nucleotide sequence of the VL is at least 85%, 86%, 87%, 88%, 89%, 90%, 91%, 92%, 93%, 94%, 95%, 96%, 97%, 98%, 99%, or 100% identical to the nucleotide sequence set forth in SEQ ID NO: 27.

In some embodiments, the nucleotide sequence of the VH is at least 85%, 86%, 87%, 88%, 89%, 90%, 91%, 92%, 93%, 94%, 95%, 96%, 97%, 98%, 99%, or 100% identical to the nucleotide sequence set forth in SEQ ID NO: 18; and the nucleotide sequence of the VL is at least 85%, 86%, 87%, 88%, 89%, 90%, 91%, 92%, 93%, 94%, 95%, 96%, 97%, 98%, 99%, or 100% identical to the nucleotide sequence set forth in SEQ ID NO: 24.

In some embodiments, the nucleotide sequence of the VH is at least 85%, 86%, 87%, 88%, 89%, 90%, 91%, 92%, 93%, 94%, 95%, 96%, 97%, 98%, 99%, or 100% identical to the nucleotide sequence set forth in SEQ ID NO: 18; and the nucleotide sequence of the VL is at least 85%, 86%, 87%, 88%, 89%, 90%, 91%, 92%, 93%, 94%, 95%, 96%, 97%, 98%, 99%, or 100% identical to the nucleotide sequence set forth in SEQ ID NO: 25.

In some embodiments, the nucleotide sequence of the VH is at least 85%, 86%, 87%, 88%, 89%, 90%, 91%, 92%, 93%, 94%, 95%, 96%, 97%, 98%, 99%, or 100% identical to the nucleotide sequence set forth in SEQ ID NO: 18; and the nucleotide sequence of the VL is at least 85%, 86%, 87%, 88%, 89%, 90%, 91%, 92%, 93%, 94%, 95%, 96%, 97%, 98%, 99%, or 100% identical to the nucleotide sequence set forth in SEQ ID NO: 26.

In some embodiments, the nucleotide sequence of the VH is at least 85%, 86%, 87%, 88%, 89%, 90%, 91%, 92%, 93%, 94%, 95%, 96%, 97%, 98%, 99%, or 100% identical to the nucleotide sequence set forth in SEQ ID NO: 18; and the nucleotide sequence of the VL is at least 85%, 86%, 87%, 88%, 89%, 90%, 91%, 92%, 93%, 94%, 95%, 96%, 97%, 98%, 99%, or 100% identical to the nucleotide sequence set forth in SEQ ID NO: 27.

In some embodiments, the nucleotide sequence of the VH is at least 85%, 86%, 87%, 88%, 89%, 90%, 91%, 92%, 93%, 94%, 95%, 96%, 97%, 98%, 99%, or 100% identical to the nucleotide sequence set forth in SEQ ID NO: 18; and the nucleotide sequence of the VL is at least 85%, 86%, 87%, 88%, 89%, 90%, 91%, 92%, 93%, 94%, 95%, 96%, 97%, 98%, 99%, or 100% identical to the nucleotide sequence set forth in SEQ ID NO: 28.

In some embodiments, the nucleotide sequence of the VH is at least 85%, 86%, 87%, 88%, 89%, 90%, 91%, 92%, 93%, 94%, 95%, 96%, 97%, 98%, 99%, or 100% identical to the nucleotide sequence set forth in SEQ ID NO: 18; and the nucleotide sequence of the VL is at least 85%, 86%, 87%, 88%, 89%, 90%, 91%, 92%, 93%, 94%, 95%, 96%, 97%, 98%, 99%, or 100% identical to the nucleotide sequence set forth in SEQ ID NO: 29.

In some embodiments, the nucleotide sequence of the VH is at least 85%, 86%, 87%, 88%, 89%, 90%, 91%, 92%, 93%, 94%, 95%, 96%, 97%, 98%, 99%, or 100% identical to the nucleotide sequence set forth in SEQ ID NO: 19; and the nucleotide sequence of the VL is at least 85%, 86%, 87%, 88%, 89%, 90%, 91%, 92%, 93%, 94%, 95%, 96%, 97%, 98%, 99%, or 100% identical to the nucleotide sequence set forth in SEQ ID NO: 24.

In some embodiments, the nucleotide sequence of the VH is at least 85%, 86%, 87%, 88%, 89%, 90%, 91%, 92%, 93%, 94%, 95%, 96%, 97%, 98%, 99%, or 100% identical to the nucleotide sequence set forth in SEQ ID NO: 19; and the nucleotide sequence of the VL is at least 85%, 86%, 87%, 88%, 89%, 90%, 91%, 92%, 93%, 94%, 95%, 96%, 97%, 98%, 99%, or 100% identical to the nucleotide sequence set forth in SEQ ID NO: 25.

In some embodiments, the nucleotide sequence of the VH is at least 85%, 86%, 87%, 88%, 89%, 90%, 91%, 92%, 93%, 94%, 95%, 96%, 97%, 98%, 99%, or 100% identical to the nucleotide sequence set forth in SEQ ID NO: 19; and the nucleotide sequence of the VL is at least 85%, 86%, 87%, 88%, 89%, 90%, 91%, 92%, 93%, 94%, 95%, 96%, 97%, 98%, 99%, or 100% identical to the nucleotide sequence set forth in SEQ ID NO: 26.

In some embodiments, the nucleotide sequence of the VH is at least 85%, 86%, 87%, 88%, 89%, 90%, 91%, 92%, 93%, 94%, 95%, 96%, 97%, 98%, 99%, or 100% identical to the nucleotide sequence set forth in SEQ ID NO: 19; and the nucleotide sequence of the VL is at least 85%, 86%, 87%, 88%, 89%, 90%, 91%, 92%, 93%, 94%, 95%, 96%, 97%, 98%, 99%, or 100% identical to the nucleotide sequence set forth in SEQ ID NO: 27.

In some embodiments, the nucleotide sequence of the VH is at least 85%, 86%, 87%, 88%, 89%, 90%, 91%, 92%, 93%, 94%, 95%, 96%, 97%, 98%, 99%, or 100% identical to the nucleotide sequence set forth in SEQ ID NO: 19; and the nucleotide sequence of the VL is at least 85%, 86%, 87%, 88%, 89%, 90%, 91%, 92%, 93%, 94%, 95%, 96%, 97%, 98%, 99%, or 100% identical to the nucleotide sequence set forth in SEQ ID NO: 28.

In some embodiments, the nucleotide sequence of the VH is at least 85%, 86%, 87%, 88%, 89%, 90%, 91%, 92%, 93%, 94%, 95%, 96%, 97%, 98%, 99%, or 100% identical to the nucleotide sequence set forth in SEQ ID NO: 19; and the nucleotide sequence of the VL is at least 85%, 86%, 87%, 88%, 89%, 90%, 91%, 92%, 93%, 94%, 95%, 96%, 97%, 98%, 99%, or 100% identical to the nucleotide sequence set forth in SEQ ID NO: 29.

In some embodiments, the nucleotide sequence of the VH is at least 85%, 86%, 87%, 88%, 89%, 90%, 91%, 92%, 93%, 94%, 95%, 96%, 97%, 98%, 99%, or 100% identical to the nucleotide sequence set forth in SEQ ID NO: 20; and the nucleotide sequence of the VL is at least 85%, 86%, 87%, 88%, 89%, 90%, 91%, 92%, 93%, 94%, 95%, 96%, 97%, 98%, 99%, or 100% identical to the nucleotide sequence set forth in SEQ ID NO: 24.

In some embodiments, the nucleotide sequence of the VH is at least 85%, 86%, 87%, 88%, 89%, 90%, 91%, 92%, 93%, 94%, 95%, 96%, 97%, 98%, 99%, or 100% identical to the nucleotide sequence set forth in SEQ ID NO: 20; and the nucleotide sequence of the VL is at least 85%, 86%, 87%, 88%, 89%, 90%, 91%, 92%, 93%, 94%, 95%, 96%, 97%, 98%, 99%, or 100% identical to the nucleotide sequence set forth in SEQ ID NO: 25.

In some embodiments, the nucleotide sequence of the VH is at least 85%, 86%, 87%, 88%, 89%, 90%, 91%, 92%, 93%, 94%, 95%, 96%, 97%, 98%, 99%, or 100% identical to the nucleotide sequence set forth in SEQ ID NO: 20; and the nucleotide sequence of the VL is at least 85%, 86%, 87%, 88%, 89%, 90%, 91%, 92%, 93%, 94%, 95%, 96%, 97%, 98%, 99%, or 100% identical to the nucleotide sequence set forth in SEQ ID NO: 26.

In some embodiments, the nucleotide sequence of the VH is at least 85%, 86%, 87%, 88%, 89%, 90%, 91%, 92%, 93%, 94%, 95%, 96%, 97%, 98%, 99%, or 100% identical to the nucleotide sequence set forth in SEQ ID NO: 20; and the nucleotide sequence of the VL is at least 85%, 86%, 87%, 88%, 89%, 90%, 91%, 92%, 93%, 94%, 95%, 96%, 97%, 98%, 99%, or 100% identical to the nucleotide sequence set forth in SEQ ID NO: 27.

In some embodiments, the nucleotide sequence of the VH is at least 85%, 86%, 87%, 88%, 89%, 90%, 91%, 92%, 93%, 94%, 95%, 96%, 97%, 98%, 99%, or 100% identical to the nucleotide sequence set forth in SEQ ID NO: 20; and the nucleotide sequence of the VL is at least 85%, 86%, 87%, 88%, 89%, 90%, 91%, 92%, 93%, 94%, 95%, 96%, 97%, 98%, 99%, or 100% identical to the nucleotide sequence set forth in SEQ ID NO: 28.

In some embodiments, the nucleotide sequence of the VH is at least 85%, 86%, 87%, 88%, 89%, 90%, 91%, 92%, 93%, 94%, 95%, 96%, 97%, 98%, 99%, or 100% identical to the nucleotide sequence set forth in SEQ ID NO: 20; and the nucleotide sequence of the VL is at least 85%, 86%, 87%, 88%, 89%, 90%, 91%, 92%, 93%, 94%, 95%, 96%, 97%, 98%, 99%, or 100% identical to the nucleotide sequence set forth in SEQ ID NO: 29.

In some embodiments, the nucleotide sequence of the VH is at least 85%, 86%, 87%, 88%, 89%, 90%, 91%, 92%, 93%, 94%, 95%, 96%, 97%, 98%, 99%, or 100% identical to the nucleotide sequence set forth in SEQ ID NO: 21; and the nucleotide sequence of the VL is at least 85%, 86%, 87%, 88%, 89%, 90%, 91%, 92%, 93%, 94%, 95%, 96%, 97%, 98%, 99%, or 100% identical to the nucleotide sequence set forth in SEQ ID NO: 24.

In some embodiments, the nucleotide sequence of the VH is at least 85%, 86%, 87%, 88%, 89%, 90%, 91%, 92%, 93%, 94%, 95%, 96%, 97%, 98%, 99%, or 100% identical to the nucleotide sequence set forth in SEQ ID NO: 21; and the nucleotide sequence of the VL is at least 85%, 86%, 87%, 88%, 89%, 90%, 91%, 92%, 93%, 94%, 95%, 96%, 97%, 98%, 99%, or 100% identical to the nucleotide sequence set forth in SEQ ID NO: 25.

In some embodiments, the nucleotide sequence of the VH is at least 85%, 86%, 87%, 88%, 89%, 90%, 91%, 92%, 93%, 94%, 95%, 96%, 97%, 98%, 99%, or 100% identical to the nucleotide sequence set forth in SEQ ID NO: 21; and the nucleotide sequence of the VL is at least 85%, 86%, 87%, 88%, 89%, 90%, 91%, 92%, 93%, 94%, 95%, 96%, 97%, 98%, 99%, or 100% identical to the nucleotide sequence set forth in SEQ ID NO: 26.

In some embodiments, the nucleotide sequence of the VH is at least 85%, 86%, 87%, 88%, 89%, 90%, 91%, 92%, 93%, 94%, 95%, 96%, 97%, 98%, 99%, or 100% identical to the nucleotide sequence set forth in SEQ ID NO: 21; and the nucleotide sequence of the VL is at least 85%, 86%, 87%, 88%, 89%, 90%, 91%, 92%, 93%, 94%, 95%, 96%, 97%, 98%, 99%, or 100% identical to the nucleotide sequence set forth in SEQ ID NO: 27.

In some embodiments, the nucleotide sequence of the VH is at least 85%, 86%, 87%, 88%, 89%, 90%, 91%, 92%, 93%, 94%, 95%, 96%, 97%, 98%, 99%, or 100% identical to the nucleotide sequence set forth in SEQ ID NO: 21; and the nucleotide sequence of the VL is at least 85%, 86%, 87%, 88%, 89%, 90%, 91%, 92%, 93%, 94%, 95%, 96%, 97%, 98%, 99%, or 100% identical to the nucleotide sequence set forth in SEQ ID NO: 28.

In some embodiments, the nucleotide sequence of the VH is at least 85%, 86%, 87%, 88%, 89%, 90%, 91%, 92%, 93%, 94%, 95%, 96%, 97%, 98%, 99%, or 100% identical to the nucleotide sequence set forth in SEQ ID NO: 21; and the nucleotide sequence of the VL is at least 85%, 86%, 87%, 88%, 89%, 90%, 91%, 92%, 93%, 94%, 95%, 96%, 97%, 98%, 99%, or 100% identical to the nucleotide sequence set forth in SEQ ID NO: 29.

In some embodiments, the nucleotide sequence of the VH is at least 85%, 86%, 87%, 88%, 89%, 90%, 91%, 92%, 93%, 94%, 95%, 96%, 97%, 98%, 99%, or 100% identical to the nucleotide sequence set forth in SEQ ID NO: 22; and the nucleotide sequence of the VL is at least 85%, 86%, 87%, 88%, 89%, 90%, 91%, 92%, 93%, 94%, 95%, 96%, 97%, 98%, 99%, or 100% identical to the nucleotide sequence set forth in SEQ ID NO: 24.

In some embodiments, the nucleotide sequence of the VH is at least 85%, 86%, 87%, 88%, 89%, 90%, 91%, 92%, 93%, 94%, 95%, 96%, 97%, 98%, 99%, or 100% identical to the nucleotide sequence set forth in SEQ ID NO: 22; and the nucleotide sequence of the VL is at least 85%, 86%, 87%, 88%, 89%, 90%, 91%, 92%, 93%, 94%, 95%, 96%, 97%, 98%, 99%, or 100% identical to the nucleotide sequence set forth in SEQ ID NO: 25.

In some embodiments, the nucleotide sequence of the VH is at least 85%, 86%, 87%, 88%, 89%, 90%, 91%, 92%, 93%, 94%, 95%, 96%, 97%, 98%, 99%, or 100% identical to the nucleotide sequence set forth in SEQ ID NO: 22; and the nucleotide sequence of the VL is at least 85%, 86%, 87%, 88%, 89%, 90%, 91%, 92%, 93%, 94%, 95%, 96%, 97%, 98%, 99%, or 100% identical to the nucleotide sequence set forth in SEQ ID NO: 26.

In some embodiments, the nucleotide sequence of the VH is at least 85%, 86%, 87%, 88%, 89%, 90%, 91%, 92%, 93%, 94%, 95%, 96%, 97%, 98%, 99%, or 100% identical to the nucleotide sequence set forth in SEQ ID NO: 22; and the nucleotide sequence of the VL is at least 85%, 86%, 87%, 88%, 89%, 90%, 91%, 92%, 93%, 94%, 95%, 96%, 97%, 98%, 99%, or 100% identical to the nucleotide sequence set forth in SEQ ID NO: 27.

In some embodiments, the nucleotide sequence of the VH is at least 85%, 86%, 87%, 88%, 89%, 90%, 91%, 92%, 93%, 94%, 95%, 96%, 97%, 98%, 99%, or 100% identical to the nucleotide sequence set forth in SEQ ID NO: 22; and the nucleotide sequence of the VL is at least 85%, 86%, 87%, 88%, 89%, 90%, 91%, 92%, 93%, 94%, 95%, 96%, 97%, 98%, 99%, or 100% identical to the nucleotide sequence set forth in SEQ ID NO: 28.

In some embodiments, the nucleotide sequence of the VH is at least 85%, 86%, 87%, 88%, 89%, 90%, 91%, 92%, 93%, 94%, 95%, 96%, 97%, 98%, 99%, or 100% identical to the nucleotide sequence set forth in SEQ ID NO: 22; and the nucleotide sequence of the VL is at least 85%, 86%, 87%, 88%, 89%, 90%, 91%, 92%, 93%, 94%, 95%, 96%, 97%, 98%, 99%, or 100% identical to the nucleotide sequence set forth in SEQ ID NO: 29.

In some embodiments, the nucleotide sequence of the VH is at least 85%, 86%, 87%, 88%, 89%, 90%, 91%, 92%, 93%, 94%, 95%, 96%, 97%, 98%, 99%, or 100% identical to the nucleotide sequence set forth in SEQ ID NO: 23; and the nucleotide sequence of the VL is at least 85%, 86%, 87%, 88%, 89%, 90%, 91%, 92%, 93%, 94%, 95%, 96%, 97%, 98%, 99%, or 100% identical to the nucleotide sequence set forth in SEQ ID NO: 24.

In some embodiments, the nucleotide sequence of the VH is at least 85%, 86%, 87%, 88%, 89%, 90%, 91%, 92%, 93%, 94%, 95%, 96%, 97%, 98%, 99%, or 100% identical to the nucleotide sequence set forth in SEQ ID NO: 23; and the nucleotide sequence of the VL is at least 85%, 86%, 87%, 88%, 89%, 90%, 91%, 92%, 93%, 94%, 95%, 96%, 97%, 98%, 99%, or 100% identical to the nucleotide sequence set forth in SEQ ID NO: 25.

In some embodiments, the nucleotide sequence of the VH is at least 85%, 86%, 87%, 88%, 89%, 90%, 91%, 92%, 93%, 94%, 95%, 96%, 97%, 98%, 99%, or 100% identical to the nucleotide sequence set forth in SEQ ID NO: 23; and the nucleotide sequence of the VL is at least 85%, 86%, 87%, 88%, 89%, 90%, 91%, 92%, 93%, 94%, 95%, 96%, 97%, 98%, 99%, or 100% identical to the nucleotide sequence set forth in SEQ ID NO: 26.

In some embodiments, the nucleotide sequence of the VH is at least 85%, 86%, 87%, 88%, 89%, 90%, 91%, 92%, 93%, 94%, 95%, 96%, 97%, 98%, 99%, or 100% identical to the nucleotide sequence set forth in SEQ ID NO: 23; and the nucleotide sequence of the VL is at least 85%, 86%, 87%, 88%, 89%, 90%, 91%, 92%, 93%, 94%, 95%, 96%, 97%, 98%, 99%, or 100% identical to the nucleotide sequence set forth in SEQ ID NO: 27.

In some embodiments, the nucleotide sequence of the VH is at least 85%, 86%, 87%, 88%, 89%, 90%, 91%, 92%, 93%, 94%, 95%, 96%, 97%, 98%, 99%, or 100% identical to the nucleotide sequence set forth in SEQ ID NO: 23; and the nucleotide sequence of the VL is at least 85%, 86%, 87%, 88%, 89%, 90%, 91%, 92%, 93%, 94%, 95%, 96%, 97%, 98%, 99%, or 100% identical to the nucleotide sequence set forth in SEQ ID NO: 28.

In some embodiments, the nucleotide sequence of the VH is at least 85%, 86%, 87%, 88%, 89%, 90%, 91%, 92%, 93%, 94%, 95%, 96%, 97%, 98%, 99%, or 100% identical to the nucleotide sequence set forth in SEQ ID NO: 23; and the nucleotide sequence of the VL is at least 85%, 86%, 87%, 88%, 89%, 90%, 91%, 92%, 93%, 94%, 95%, 96%, 97%, 98%, 99%, or 100% identical to the nucleotide sequence set forth in SEQ ID NO: 29.

### 5.2 hIL-12 Proteins & Polypeptides

hIL-12 is a pleotropic secreted cytokine composed of an a subunit, hIL-12p35, and a β subunit, hIL-12p40. The naturally occurring hIL-12p35 and hIL-12p40 subunits are linked through a disulfide bond to form the bioactive hIL12-p70 cytokine. See, e.g., Sun, Lin et al. “Interleukin 12 (IL-12) family cytokines: Role in immune pathogenesis and treatment of CNS autoimmune disease,” Cytokine, 75 (2): 249-55 (2015) (hereinafter “Sun 2015”), the entire contents of which is incorporated herein by reference for all purposes. hIL-12 is, inter alia, pro-inflammatory, and mediates its functions through binding to the hIL-12 receptor (hIL-12R). The high affinity hIL-12R is a heterodimeric comprising a hIL-12Rβ1 subunit and a hIL-12Rβ2 subunit. The hIL-12R is expressed in a constitutive or inducible manner in a variety of immune cells, including NK cells, T-cells, and B-cells. Binding of hIL-12 to the hIL-12R expressed on e.g., activated T cells, NK cells and DCs, activates TYK2, JAK2, and STAT pathways. Among the STAT family of transcription factors, STAT4 is considered to be the most specific mediator of cellular responses elicited by hIL-12. See, e.g., Sun 2015.

The amino acid sequence of a reference immature hIL-12p35 polypeptide and mature hIL-12p35 polypeptide is set forth in SEQ ID NOS: 30 and 31, respectively. The amino acid sequence of a reference immature hIL-12p40 polypeptide and mature hIL-12p40 polypeptide is set forth in SEQ ID NOS: 32 and 33, respectively. The amino acid sequence of a reference immature human IL-12Rβ1 (hIL-12Rβ1) polypeptide and mature hIL-12Rβ1 polypeptide is set forth in SEQ ID NOS: 34 and 35, respectively. The amino acid sequence of a reference immature human IL-12Rβ2 (hIL-12Rβ12) polypeptide and mature hIL-12Rβ2 polypeptide is set forth in SEQ ID NOS: 36 and 37, respectively. See Table 4, herein.

TABLE 4
The Amino Acid Sequence of Reference hIL-12p35, hIL-12p40, hIL-12Rβ1; and
hIL-12Rβ2 Polypeptides

| | | SEQ |
|---|---|---|
| Description | Amino Acid Sequence | ID NO |
| hIL-12p35 | MCPARSLLLVATLVLLDHLSLARNLPVATPDPGMFPCLHHSQN | 30 |
| (Immature-Signal | LLRAVSNMLQKARQTLEFYPCTSEEIDHEDITKDKTSTVEACL | |
| Peptide Underlined) | PLELTKNESCLNSRETSFITNGSCLASRKTSFMMALCLSSIYE | |
| UniProt ID: P29459 | DLKMYQVEFKTMNAKLLMDPKRQIFLDQNMLAVIDELMQALNF | |
| | NSETVPQKSSLEEPDFYKTKIKLCILLHAFRIRAVTIDRVMSY | |
| | LNAS | |
| hIL-12p35 | RNLPVATPDPGMFPCLHHSQNLLRAVSNMLQKARQTLEFYPCT | 31 |
| (Mature-No Signal | SEEIDHEDITKDKTSTVEACLPLELTKNESCLNSRETSFITNG | |
| Peptide) | SCLASRKTSFMMALCLSSIYEDLKMYQVEFKTMNAKLLMDPKR | |
| UniProt ID: P29459 | QIFLDQNMLAVIDELMQALNFNSETVPQKSSLEEPDFYKTKIK | |
| | LCILLHAFRIRAVTIDRVMSYLNAS | |
| hIL-12p40 | MCHQQLVISWFSLVFLASPLVAIWELKKDVYVVELDWYPDAPG | 32 |
| (Immature-Signal | EMVVLTCDTPEEDGITWTLDQSSEVLGSGKTLTIQVKEFGDAG | |
| Peptide Underlined) | QYTCHKGGEVLSHSLLLLHKKEDGIWSTDILKDQKEPKNKTFL | |
| UniProt ID: P29460 | RCEAKNYSGRFTCWWLTTISTDLTFSVKSSRGSSDPQGVTCGA | |
| | ATLSAERVRGDNKEYEYSVECQEDSACPAAEESLPIEVMVDAV | |
| | HKLKYENYTSSFFIRDIIKPDPPKNLQLKPLKNSRQVEVSWEY | |
| | PDTWSTPHSYFSLTFCVQVQGKSKREKKDRVFTDKTSATVICR | |
| | KNASISVRAQDRYYSSSWSEWASVPCS | |
| hIL-12p40 | IWELKKDVYVVELDWYPDAPGEMVVLTCDTPEEDGITWTLDQS | 33 |
| (Mature-No Signal | SEVLGSGKTLTIQVKEFGDAGQYTCHKGGEVLSHSLLLLHKKE | |
| Peptide) | DGIWSTDILKDQKEPKNKTFLRCEAKNYSGRFTCWWLTTISTD | |
| UniProt ID: P29460 | LTFSVKSSRGSSDPQGVTCGAATLSAERVRGDNKEYEYSVECQ | |
| | EDSACPAAEESLPIEVMVDAVHKLKYENYTSSFFIRDIIKPDP | |
| | PKNLQLKPLKNSRQVEVSWEYPDTWSTPHSYFSLTFCVQVQGK | |
| | SKREKKDRVFTDKTSATVICRKNASISVRAQDRYYSSSWSEWA | |
| | SVPCS | |
| hIL-12Rß1 | MEPLVTWVVPLLFLFLLSRQGAACRTSECCFQDPPYPDADSGS | 34 |
| (Immature-Signal | ASGPRDLRCYRISSDRYECSWQYEGPTAGVSHFLRCCLSSGRC | |
| Peptide Underlined) | CYFAAGSATRLQFSDQAGVSVLYTVTLWVESWARNQTEKSPEV | |
| UniProt ID: P42701 | TLQLYNSVKYEPPLGDIKVSKLAGQLRMEWETPDNQVGAEVQF | |
| | RHRTPSSPWKLGDCGPQDDDTESCLCPLEMNVAQEFQLRRRQL | |
| | GSQGSSWSKWSSPVCVPPENPPQPQVRFSVEQLGQDGRRRLTL | |
| | KEQPTQLELPEGCQGLAPGTEVTYRLQLHMLSCPCKAKATRTL | |
| | HLGKMPYLSGAAYNVAVISSNQFGPGLNQTWHIPADTHTEPVA | |
| | LNISVGTNGTTMYWPARAQSMTYCIEWQPVGQDGGLATCSLTA | |
| | PQDPDPAGMATYSWSRESGAMGQEKCYYITIFASAHPEKLTLW | |
| | STVLSTYHFGGNASAAGTPHHVSVKNHSLDSVSVDWAPSLLST | |
| | CPGVLKEYVVRCRDEDSKQVSEHPVQPTETQVTLSGLRAGVAY | |
| | TVQVRADTAWLRGVWSQPQRFSIEVQVSDWLIFFASLGSFLSI | |
| | LLVGVLGYLGLNRAARHLCPPLPTPCASSAIEFPGGKETWQWI | |
| | NPVDFQEEASLQEALVVEMSWDKGERTEPLEKTELPEGAPELA | |
| | LDTELSLEDGDRCKAKM | |
| hIL-12Rß1 | CRTSECCFQDPPYPDADSGSASGPRDLRCYRISSDRYECSWQY | 35 |
| (Mature-No Signal | EGPTAGVSHFLRCCLSSGRCCYFAAGSATRLQFSDQAGVSVLY | |
| Peptide) | TVTLWVESWARNQTEKSPEVTLQLYNSVKYEPPLGDIKVSKLA | |
| UniProt ID: P42701 | GQLRMEWETPDNQVGAEVQFRHRTPSSPWKLGDCGPQDDDTES | |
| | CLCPLEMNVAQEFQLRRRQLGSQGSSWSKWSSPVCVPPENPPQ | |
| | PQVRFSVEQLGQDGRRRLTLKEQPTQLELPEGCQGLAPGTEVT | |
| | YRLQLHMLSCPCKAKATRTLHLGKMPYLSGAAYNVAVISSNQF | |
| | GPGLNQTWHIPADTHTEPVALNISVGTNGTTMYWPARAQSMTY | |
| | CIEWQPVGQDGGLATCSLTAPQDPDPAGMATYSWSRESGAMGQ | |
| | EKCYYITIFASAHPEKLTLWSTVLSTYHFGGNASAAGTPHHVS | |
| | VKNHSLDSVSVDWAPSLLSTCPGVLKEYVVRCRDEDSKQVSEH | |
| | PVQPTETQVTLSGLRAGVAYTVQVRADTAWLRGVWSQPQRFSI | |
| | EVQVSDWLIFFASLGSFLSILLVGVLGYLGLNRAARHLCPPLP | |
| | TPCASSAIEFPGGKETWQWINPVDFQEEASLQEALVVEMSWDK | |
| | GERTEPLEKTELPEGAPELALDTELSLEDGDRCKAKM | |
| hIL-12Rβ2 | MAHTFRGCSLAFMFIITWLLIKAKIDACKRGDVTVKPSHVILL | 36 |
| (Immature-Signal | GSTVNITCSLKPRQGCFHYSRRNKLILYKFDRRINFHHGHSLN | |
| Peptide Underlined) | SQVTGLPLGTTLFVCKLACINSDEIQICGAEIFVGVAPEQPQN | |
| UniProt ID: Q99665 | LSCIQKGEQGTVACTWERGRDTHLYTEYTLQLSGPKNLTWQKQ | |
| | CKDIYCDYLDFGINLTPESPESNFTAKVTAVNSLGSSSSLPST | |
| | FTFLDIVRPLPPWDIRIKFQKASVSRCTLYWRDEGLVLLNRLR | |
| | YRPSNSRLWNMVNVTKAKGRHDLLDLKPFTEYEFQISSKLHLY | |
| | KGSWSDWSESLRAQTPEEEPTGMLDVWYMKRHIDYSRQQISLF | |
| | WKNLSVSEARGKILHYQVTLQELTGGKAMTQNITGHTSWTTVI | |
| | PRTGNWAVAVSAANSKGSSLPTRINIMNLCEAGLLAPRQVSAN | |
| | SEGMDNILVTWQPPRKDPSAVQEYVVEWRELHPGGDTQVPLNW | |
| | LRSRPYNVSALISENIKSYICYEIRVYALSGDQGGCSSILGNS | |
| | KHKAPLSGPHINAITEEKGSILISWNSIPVQEQMGCLLHYRIY | |
| | WKERDSNSQPQLCEIPYRVSQNSHPINSLQPRVTYVLWMTALT | |
| | AAGESSHGNEREFCLQGKANWMAFVAPSICIAIIMVGIFSTHY | |
| | FQQKVFVLLAALRPQWCSREIPDPANSTCAKKYPIAEEKTQLP | |
| | LDRLLIDWPTPEDPEPLVISEVLHQVTPVFRHPPCSNWPQREK | |
| | GIQGHQASEKDMMHSASSPPPPRALQAESRQLVDLYKVLESRG | |
| | SDPKPENPACPWTVLPAGDLPTHDGYLPSNIDDLPSHEAPLAD | |
| | SLEELEPQHISLSVFPSSSLHPLTFSCGDKLTLDQLKMRCDSL | |
| | ML | |
| hIL-12Rß2 | KIDACKRGDVTVKPSHVILLGSTVNITCSLKPRQGCFHYSRRN | 37 |
| (Mature-No Signal | KLILYKFDRRINFHHGHSLNSQVTGLPLGTTLFVCKLACINSD | |
| Peptide) | EIQICGAEIFVGVAPEQPQNLSCIQKGEQGTVACTWERGRDTH | |
| UniProt ID: Q99665 | LYTEYTLQLSGPKNLTWQKQCKDIYCDYLDFGINLTPESPESN | |
| | FTAKVTAVNSLGSSSSLPSTFTFLDIVRPLPPWDIRIKFQKAS | |
| | VSRCTLYWRDEGLVLLNRLRYRPSNSRLWNMVNVTKAKGRHDL | |
| | LDLKPFTEYEFQISSKLHLYKGSWSDWSESLRAQTPEEEPTGM | |
| | LDVWYMKRHIDYSRQQISLFWKNLSVSEARGKILHYQVTLQEL | |
| | TGGKAMTQNITGHTSWTTVIPRTGNWAVAVSAANSKGSSLPTR | |
| | INIMNLCEAGLLAPRQVSANSEGMDNILVTWQPPRKDPSAVQE | |
| | YVVEWRELHPGGDTQVPLNWLRSRPYNVSALISENIKSYICYE | |
| | IRVYALSGDQGGCSSILGNSKHKAPLSGPHINAITEEKGSILI | |
| | SWNSIPVQEQMGCLLHYRIYWKERDSNSQPQLCEIPYRVSQNS | |
| | HPINSLQPRVTYVLWMTALTAAGESSHGNEREFCLQGKANWMA | |
| | FVAPSICIAIIMVGIFSTHYFQQKVFVLLAALRPQWCSREIPD | |
| | PANSTCAKKYPIAEEKTQLPLDRLLIDWPTPEDPEPLVISEVL | |
| | HQVTPVFRHPPCSNWPQREKGIQGHQASEKDMMHSASSPPPPR | |
| | ALQAESRQLVDLYKVLESRGSDPKPENPACPWTVLPAGDLPTH | |
| | DGYLPSNIDDLPSHEAPLADSLEELEPQHISLSVFPSSSLHPL | |
| | TFSCGDKLTLDQLKMRCDSLML | |

5.2.1 hIL-12p40 Proteins & Polypeptides

In one aspect, provided herein are hIL-12p40 polypeptides with attenuated activity compared to a reference hIL-12p40 polypeptide (e.g., SEQ ID NO: 33). As described herein, any of the hIL-12p40 polypeptides described herein may be isolated and/or recombinant. In some embodiments, the hIL-12p40 polypeptide specifically binds the hIL-12R. In some embodiments, the hIL-12p40 polypeptide specifically binds the hIL-12Rβ1 (see also, § 5.2.3). In some embodiments, the hIL-12p40 polypeptide specifically binds the hIL-12Rβ2 (see also, § 5.2.2).

As set forth above, for the purposes of the instant disclosure, the numbering of all amino acids (and e.g., amino acid substitutions) of hIL-12p40 polypeptides described herein is set out relative to the amino acid sequence of the immature form of hIL-12p40 (i.e., SEQ ID NO: 32), that contains the native signal peptide. The use of the immature form of hIL-12p40 to designate amino acid numbers (e.g., W37) is for consistency only and does not limit the scope of embodiments utilizing this numbering scheme to polypeptides that contain the signal peptide of hIL-12p40. For example, a hIL-12p40 polypeptide described herein as comprising the amino acid sequence of SEQ ID NO: 33 with a W37A amino acid substitution does not require the signal peptide of hIL-12p40, although the numbering of amino acid position W37 is based on the immature form of the protein. It is common in the art to utilize the mature form of a protein to produce variants and fusion proteins.

A person of ordinary skill in art can easily determine the amino acid position in the mature form of hIL-12p40 (SEQ ID NO: 33) based on the amino acid numbering relative to the immature form of hIL-12p40. As set forth above, amino acids 1-22 of the immature form of the hIL-12p40 protein are the signal sequence (underlined). Therefore, an amino acid position of a particular amino acid in the mature form of the hIL-12p40 protein can be determined from the amino acid position of the particular amino acid designated relative to the immature form of hIL-12p40 by subtracting 22. For example, the amino acid position W37 (numbering relative to SEQ ID NO: 32) would correspond to amino acid position W15 in the mature form of the protein (SEQ ID NO: 33).

In one aspect, provided herein are hIL-12p40 polypeptides comprising or consisting of an amino acid sequence (a) at least 85%, 86%, 87%, 88%, 89%, 90%, 91%, 92%, 93%, 94%, 95%, 96%, 97%, 98%, or 99% identical to the amino acid sequence of SEQ ID NO: 33; and (b) comprising or consisting of an amino acid modification (e.g., substitution, addition, deletion (e.g., substitution)) at each of amino acid positions (i) W37, F82, and K219; (ii) W37, F82, and K217; (iii) K106, K217, and K219; (iv) W37 and F82; (v) W37 and K217; (vi) W37 and K219; (vii) W37 and K106; (viii) F82 and K106; (xiv) F82 and K217; (xv) F82 and K219; (xvi) K217 and K219; (xvii) K106 and K217; or (xviii) K106 and K219, amino acid numbering relative to the amino acid sequence of SEQ ID NO: 32.

In some embodiments, the hIL-12p40 polypeptides comprise or consist of an amino acid sequence (a) at least 85%, 86%, 87%, 88%, 89%, 90%, 91%, 92%, 93%, 94%, 95%, 96%, 97%, 98%, or 99% identical to the amino acid sequence of SEQ ID NO: 33; and (b) comprise or consist of an amino acid substitution at each of amino acid positions (i) W37, F82, and K219; (ii) W37, F82, and K217; (iii) K106, K217, and K219; (iv) W37 and F82; (v) W37 and K217; (vi) W37 and K219; (vii) W37 and K106; (viii) F82 and K106; (xiv) F82 and K217; (xv) F82 and K219; (xvi) K217 and K219; (xvii) K106 and K217; or (xviii) K106 and K219, amino acid numbering relative to the amino acid sequence of SEQ ID NO: 32. In some embodiments, each of the amino acid substitutions is a replacement of the native amino acid residue with an alanine.

In some embodiments, the hIL-12p40 polypeptide comprises or consists of an amino acid sequence (a) at least 85%, 86%, 87%, 88%, 89%, 90%, 91%, 92%, 93%, 94%, 95%, 96%, 97%, 98%, or 99% identical to the amino acid sequence of SEQ ID NO: 33; and (b) comprises or consists of each of the following amino acid substitutions (i) W37A, F82A, and K219A; (ii) W37A, F82A, and K217A; (iii) K106A, K217A, and K219A; (iv) W37A and F82A; (v) W37A and K217A; (vi) W37A and K219A; (vii) W37A and K106A; (viii) F82A and K106A; (xiv) F82A and K217A; (xv) F82A and K219A; (xvi) K217A and K219A; (xvii) K106A and K217A; or (xviii) K106A and K219A, amino acid numbering relative to the amino acid sequence of SEQ ID NO: 32.

In some embodiments, the hIL-12p40 polypeptide comprises or consist of an amino acid sequence (a) at least 85%, 86%, 87%, 88%, 89%, 90%, 91%, 92%, 93%, 94%, 95%, 96%, 97%, 98%, or 99% identical to the amino acid sequence of SEQ ID NO: 33; and (b) comprises or consists of an amino acid modification (e.g., substitution) at each of amino acid positions (i) W37, F82, and K219, amino acid numbering relative to the amino acid sequence of SEQ ID NO: 32.

In some embodiments, the hIL-12p40 polypeptide comprises or consist of an amino acid sequence (a) at least 85%, 86%, 87%, 88%, 89%, 90%, 91%, 92%, 93%, 94%, 95%, 96%, 97%, 98%, or 99% identical to the amino acid sequence of SEQ ID NO: 33; and (b) comprises or consists of each of the following amino acid substitutions (i) W37A, F82A, and K219A, amino acid numbering relative to the amino acid sequence of SEQ ID NO: 32.

The amino acid sequence of exemplary hIL-12p40 polypeptides described herein is provided in Table 5.

TABLE 5
The Amino Acid Sequence of Exemplary hIL-12p40 Polypeptides

| Description | SEQ ID NO | Amino Acid Sequence |
|---|---|---|
| hIL-12p40 | 38 | IWELKKDVYVVELDAYPDAPGEMVVLTCDTPEEDGITWTLD |
| Variant O | | QSSEVLGSGKTLTIQVKEAGDAGQYTCHKGGEVLSHSLLLL |
| (mature-no signal | | HKKEDGIWSTDILKDQKEPKNKTFLRCEAKNYSGRFTCWWL |
| peptide) | | TTISTDLTFSVKSSRGSSDPQGVTCGAATLSAERVRGDNKE |
| (W37A/F82A/K219A) | | YEYSVECQEDSACPAAEESLPIEVMVDAVHKLAYENYTSSF |
| | | FIRDIIKPDPPKNLQLKPLKNSRQVEVSWEYPDTWSTPHSY |
| | | FSLTFCVQVQGKSKREKKDRVFTDKTSATVICRKNASISVR |
| | | AQDRYYSSSWSEWASVPCS |
| hIL-12p40 | 39 | IWELKKDVYVVELDAYPDAPGEMVVLTCDTPEEDGITWTLD |
| Variant N | | QSSEVLGSGKTLTIQVKEAGDAGQYTCHKGGEVLSHSLLLL |
| (mature-no signal | | HKKEDGIWSTDILKDQKEPKNKTFLRCEAKNYSGRFTCWWL |
| peptide) | | TTISTDLTFSVKSSRGSSDPQGVTCGAATLSAERVRGDNKE |
| (W37A/F82A/K217A) | | YEYSVECQEDSACPAAEESLPIEVMVDAVHALKYENYTSSF |
| | | FIRDIIKPDPPKNLQLKPLKNSRQVEVSWEYPDTWSTPHSY |
| | | FSLTFCVQVQGKSKREKKDRVFTDKTSATVICRKNASISVR |
| | | AQDRYYSSSWSEWASVPCS |
| hIL-12p40 | 40 | IWELKKDVYVVELDWYPDAPGEMVVLTCDTPEEDGITWTLD |
| Variant P | | QSSEVLGSGKTLTIQVKEFGDAGQYTCHKGGEVLSHSLLLL |
| (mature-no signal | | HAKEDGIWSTDILKDQKEPKNKTFLRCEAKNYSGRFTCWWL |
| peptide) | | TTISTDLTFSVKSSRGSSDPQGVTCGAATLSAERVRGDNKE |
| (K106A/K217A/K219A) | | YEYSVECQEDSACPAAEESLPIEVMVDAVHALAYENYTSSF |
| | | FIRDIIKPDPPKNLQLKPLKNSRQVEVSWEYPDTWSTPHSY |
| | | FSLTFCVQVQGKSKREKKDRVFTDKTSATVICRKNASISVR |
| | | AQDRYYSSSWSEWASVPCS |
| hIL-12p40 | 41 | IWELKKDVYVVELDAYPDAPGEMVVLTCDTPEEDGITWTLD |
| Variant T | | QSSEVLGSGKTLTIQVKEAGDAGQYTCHKGGEVLSHSLLLL |
| (mature-no signal | | HKKEDGIWSTDILKDQKEPKNKTFLRCEAKNYSGRFTCWWL |
| peptide) | | TTISTDLTFSVKSSRGSSDPQGVTCGAATLSAERVRGDNKE |
| (W37A/F82A) | | YEYSVECQEDSACPAAEESLPIEVMVDAVHKLKYENYTSSF |
| | | FIRDIIKPDPPKNLQLKPLKNSRQVEVSWEYPDTWSTPHSY |
| | | FSLTFCVQVQGKSKREKKDRVFTDKTSATVICRKNASISVR |
| | | AQDRYYSSSWSEWASVPCS |
| hIL-12p40 | 42 | IWELKKDVYVVELDAYPDAPGEMVVLTCDTPEEDGITWTLD |
| Variant U | | QSSEVLGSGKTLTIQVKEFGDAGQYTCHKGGEVLSHSLLLL |
| (mature-no signal | | HKKEDGIWSTDILKDQKEPKNKTFLRCEAKNYSGRFTCWWL |
| peptide) | | TTISTDLTFSVKSSRGSSDPQGVTCGAATLSAERVRGDNKE |
| (W37A/K217A) | | YEYSVECQEDSACPAAEESLPIEVMVDAVHALKYENYTSSF |
| | | FIRDIIKPDPPKNLQLKPLKNSRQVEVSWEYPDTWSTPHSY |
| | | FSLTFCVQVQGKSKREKKDRVFTDKTSATVICRKNASISVR |
| | | AQDRYYSSSWSEWASVPCS |
| hIL-12p40 | 43 | IWELKKDVYVVELDAYPDAPGEMVVLTCDTPEEDGITWTLD |
| Variant V | | QSSEVLGSGKTLTIQVKEFGDAGQYTCHKGGEVLSHSLLLL |
| (mature-no signal | | HKKEDGIWSTDILKDQKEPKNKTFLRCEAKNYSGRFTCWWL |
| peptide) | | TTISTDLTFSVKSSRGSSDPQGVTCGAATLSAERVRGDNKE |
| (W37A/K219A) | | YEYSVECQEDSACPAAEESLPIEVMVDAVHKLAYENYTSSF |
| | | FIRDIIKPDPPKNLQLKPLKNSRQVEVSWEYPDTWSTPHSY |
| | | FSLTFCVQVQGKSKREKKDRVFTDKTSATVICRKNASISVR |
| | | AQDRYYSSSWSEWASVPCS |
| hIL-12p40 | 44 | IWELKKDVYVVELDAYPDAPGEMVVLTCDTPEEDGITWTLD |
| Variant W | | QSSEVLGSGKTLTIQVKEFGDAGQYTCHKGGEVLSHSLLLL |
| (mature-no signal | | HAKEDGIWSTDILKDQKEPKNKTFLRCEAKNYSGRFTCWWL |
| peptide) | | TTISTDLTFSVKSSRGSSDPQGVTCGAATLSAERVRGDNKE |
| (W37A/K106A) | | YEYSVECQEDSACPAAEESLPIEVMVDAVHKLKYENYTSSF |
| | | FIRDIIKPDPPKNLQLKPLKNSRQVEVSWEYPDTWSTPHSY |
| | | FSLTFCVQVQGKSKREKKDRVFTDKTSATVICRKNASISVR |
| | | AQDRYYSSSWSEWASVPCS |
| hIL-12p40 | 45 | IWELKKDVYVVELDWYPDAPGEMVVLTCDTPEEDGITWTLD |
| Variant X | | QSSEVLGSGKTLTIQVKEAGDAGQYTCHKGGEVLSHSLLLL |
| (mature-no signal | | HAKEDGIWSTDILKDQKEPKNKTFLRCEAKNYSGRFTCWWL |
| peptide) | | TTISTDLTFSVKSSRGSSDPQGVTCGAATLSAERVRGDNKE |
| (F82A/K106A) | | YEYSVECQEDSACPAAEESLPIEVMVDAVHKLKYENYTSSF |
| | | FIRDIIKPDPPKNLQLKPLKNSRQVEVSWEYPDTWSTPHSY |
| | | FSLTFCVQVQGKSKREKKDRVFTDKTSATVICRKNASISVR |
| | | AQDRYYSSSWSEWASVPCS |
| hIL-12p40 | 46 | IWELKKDVYVVELDWYPDAPGEMVVLTCDTPEEDGITWTLD |
| Variant Y | | QSSEVLGSGKTLTIQVKEAGDAGQYTCHKGGEVLSHSLLLL |
| (mature-no signal | | HKKEDGIWSTDILKDQKEPKNKTFLRCEAKNYSGRFTCWWL |
| peptide) | | TTISTDLTFSVKSSRGSSDPQGVTCGAATLSAERVRGDNKE |
| (F82A/K217A) | | YEYSVECQEDSACPAAEESLPIEVMVDAVHALKYENYTSSF |
| | | FIRDIIKPDPPKNLQLKPLKNSRQVEVSWEYPDTWSTPHSY |
| | | FSLTFCVQVQGKSKREKKDRVFTDKTSATVICRKNASISVR |
| | | AQDRYYSSSWSEWASVPCS |
| hIL-12p40 | 47 | IWELKKDVYVVELDWYPDAPGEMVVLTCDTPEEDGITWTLD |
| Variant Z | | QSSEVLGSGKTLTIQVKEAGDAGQYTCHKGGEVLSHSLLLL |
| (mature-no signal | | HKKEDGIWSTDILKDQKEPKNKTFLRCEAKNYSGRFTCWWL |
| peptide) | | TTISTDLTFSVKSSRGSSDPQGVTCGAATLSAERVRGDNKE |
| (F82A/K219A) | | YEYSVECQEDSACPAAEESLPIEVMVDAVHKLAYENYTSSF |
| | | FIRDIIKPDPPKNLQLKPLKNSRQVEVSWEYPDTWSTPHSY |
| | | FSLTFCVQVQGKSKREKKDRVFTDKTSATVICRKNASISVR |
| | | AQDRYYSSSWSEWASVPCS |
| hIL-12p40 | 48 | IWELKKDVYVVELDWYPDAPGEMVVLTCDTPEEDGITWTLD |
| Variant AA | | QSSEVLGSGKTLTIQVKEFGDAGQYTCHKGGEVLSHSLLLL |
| (mature-no signal | | HKKEDGIWSTDILKDQKEPKNKTFLRCEAKNYSGRFTCWWL |
| peptide) | | TTISTDLTFSVKSSRGSSDPQGVTCGAATLSAERVRGDNKE |
| (K217A/K219A) | | YEYSVECQEDSACPAAEESLPIEVMVDAVHALAYENYTSSF |
| | | FIRDIIKPDPPKNLQLKPLKNSRQVEVSWEYPDTWSTPHSY |
| | | FSLTFCVQVQGKSKREKKDRVFTDKTSATVICRKNASISVR |
| | | AQDRYYSSSWSEWASVPCS |
| hIL-12p40 | 49 | IWELKKDVYVVELDWYPDAPGEMVVLTCDTPEEDGITWTLD |
| Variant BB | | QSSEVLGSGKTLTIQVKEFGDAGQYTCHKGGEVLSHSLLLL |
| (mature-no signal | | HAKEDGIWSTDILKDQKEPKNKTFLRCEAKNYSGRFTCWWL |
| peptide) | | TTISTDLTFSVKSSRGSSDPQGVTCGAATLSAERVRGDNKE |
| (K106A/K217A) | | YEYSVECQEDSACPAAEESLPIEVMVDAVHALKYENYTSSF |
| | | FIRDIIKPDPPKNLQLKPLKNSRQVEVSWEYPDTWSTPHSY |
| | | FSLTFCVQVQGKSKREKKDRVFTDKTSATVICRKNASISVR |
| | | AQDRYYSSSWSEWASVPCS |
| hIL-12p40 | 50 | IWELKKDVYVVELDWYPDAPGEMVVLTCDTPEEDGITWTLD |
| Variant CC | | QSSEVLGSGKTLTIQVKEFGDAGQYTCHKGGEVLSHSLLLL |
| (mature-no signal | | HAKEDGIWSTDILKDQKEPKNKTFLRCEAKNYSGRFTCWWL |
| peptide) | | TTISTDLTFSVKSSRGSSDPQGVTCGAATLSAERVRGDNKE |
| (K106A/K219A) | | YEYSVECQEDSACPAAEESLPIEVMVDAVHKLAYENYTSSF |
| | | FIRDIIKPDPPKNLQLKPLKNSRQVEVSWEYPDTWSTPHSY |
| | | FSLTFCVQVQGKSKREKKDRVFTDKTSATVICRKNASISVR |
| | | AQDRYYSSSWSEWASVPCS |
| hIL-12p40 | 51 | IWELKKDVYVVELDWYPDAPGEMVVLTCDTPEEDGITWTLD |
| Variant H | | QSSEVLGSGKTLTIQVKEFGDAGQYTCHKGGEVLSHSLLLL |
| (mature-no signal | | HKKEDGIWSTDILKDQKEPKNKTFLRCEAKNYSGRFTCWWL |
| peptide) | | TTISTDLTFSVKSSRGSSDPQGVTCGAATLSAERVRGDNKE |
| (K217 + L218 | | YEYSVECQEDSACPAAEESLPIEVMVDAVHIKYENYTSSFF |
| substituted with I) | | IRDIIKPDPPKNLQLKPLKNSRQVEVSWEYPDTWSTPHSYF |
| | | SLTFCVQVQGKSKREKKDRVFTDKTSATVICRKNASISVRA |
| | | QDRYYSSSWSEWASVPCS |
| hIL-12p40 | 52 | MCHQQLVISWFSLVFLASPLVAIWELKKDVYVVELDAYPDA |
| Variant O | | PGEMVVLTCDTPEEDGITWTLDQSSEVLGSGKTLTIQVKEA |
| (immature-signal | | GDAGQYTCHKGGEVLSHSLLLLHKKEDGIWSTDILKDQKEP |
| peptide underlined) | | KNKTFLRCEAKNYSGRFTCWWLTTISTDLTESVKSSRGSSD |
| (W37A/F82A/K219A) | | PQGVTCGAATLSAERVRGDNKEYEYSVECQEDSACPAAEES |
| | | LPIEVMVDAVHKLAYENYTSSFFIRDIIKPDPPKNLQLKPL |
| | | KNSRQVEVSWEYPDTWSTPHSYFSLTFCVQVQGKSKREKKD |
| | | RVFTDKTSATVICRKNASISVRAQDRYYSSSWSEWASVPCS |
| hIL-12p40 | 53 | MCHQQLVISWESLVELASPLVAIWELKKDVYVVELDAYPDA |
| Variant N | | PGEMVVLTCDTPEEDGITWTLDQSSEVLGSGKTLTIQVKEA |
| (immature-signal | | GDAGQYTCHKGGEVLSHSLLLLHKKEDGIWSTDILKDQKEP |
| peptide underlined) | | KNKTFLRCEAKNYSGRFTCWWLTTISTDLTFSVKSSRGSSD |
| (W37A/F82A/K217A) | | PQGVTCGAATLSAERVRGDNKEYEYSVECQEDSACPAAEES |
| | | LPIEVMVDAVHALKYENYTSSFFIRDIIKPDPPKNLQLKPL |
| | | KNSRQVEVSWEYPDTWSTPHSYFSLTFCVQVQGKSKREKKD |
| | | RVFTDKTSATVICRKNASISVRAQDRYYSSSWSEWASVPCS |
| hIL-12p40 | 54 | MCHQQLVISWESLVELASPLVAIWELKKDVYVVELDWYPDA |
| Variant P | | PGEMVVLTCDTPEEDGITWTLDQSSEVLGSGKTLTIQVKEF |
| (immature-signal | | GDAGQYTCHKGGEVLSHSLLLLHAKEDGIWSTDILKDQKEP |
| peptide underlined) | | KNKTFLRCEAKNYSGRFTCWWLTTISTDLTFSVKSSRGSSD |
| (K106A/K217A/K219A) | | PQGVTCGAATLSAERVRGDNKEYEYSVECQEDSACPAAEES |
| | | LPIEVMVDAVHALAYENYTSSFFIRDIIKPDPPKNLQLKPL |
| | | KNSRQVEVSWEYPDTWSTPHSYFSLTFCVQVQGKSKREKKD |
| | | RVFTDKTSATVICRKNASISVRAQDRYYSSSWSEWASVPCS |
| hIL-12p40 | 55 | MCHQQLVISWESLVELASPLVAIWELKKDVYVVELDAYPDA |
| Variant T | | PGEMVVLTCDTPEEDGITWTLDQSSEVLGSGKTLTIQVKEA |
| (immature-signal | | GDAGQYTCHKGGEVLSHSLLLLHKKEDGIWSTDILKDQKEP |
| peptide underlined) | | KNKTFLRCEAKNYSGRFTCWWLTTISTDLTFSVKSSRGSSD |
| (W37A/F82A) | | PQGVTCGAATLSAERVRGDNKEYEYSVECQEDSACPAAEES |
| | | LPIEVMVDAVHKLKYENYTSSFFIRDIIKPDPPKNLQLKPL |
| | | KNSRQVEVSWEYPDTWSTPHSYFSLTFCVQVQGKSKREKKD |
| | | RVFTDKTSATVICRKNASISVRAQDRYYSSSWSEWASVPCS |
| hIL-12p40 | 56 | MCHQQLVISWFSLVFLASPLVAIWELKKDVYVVELDAYPDA |
| Variant U | | PGEMVVLTCDTPEEDGITWTLDQSSEVLGSGKTLTIQVKEF |
| (immature-signal | | GDAGQYTCHKGGEVLSHSLLLLHKKEDGIWSTDILKDQKEP |
| peptide underlined) | | KNKTFLRCEAKNYSGRFTCWWLTTISTDLTFSVKSSRGSSD |
| (W37A/K217A) | | PQGVTCGAATLSAERVRGDNKEYEYSVECQEDSACPAAEES |
| | | LPIEVMVDAVHALKYENYTSSFFIRDIIKPDPPKNLQLKPL |
| | | KNSRQVEVSWEYPDTWSTPHSYFSLTFCVQVQGKSKREKKD |
| | | RVFTDKTSATVICRKNASISVRAQDRYYSSSWSEWASVPCS |
| hIL-12p40 | 57 | MCHQQLVISWFSLVFLASPLVAIWELKKDVYVVELDAYPDA |
| Variant V | | PGEMVVLTCDTPEEDGITWTLDQSSEVLGSGKTLTIQVKEF |
| (immature-signal | | GDAGQYTCHKGGEVLSHSLLLLHKKEDGIWSTDILKDQKEP |
| peptide underlined) | | KNKTFLRCEAKNYSGRFTCWWLTTISTDLTFSVKSSRGSSD |
| (W37A/K219A) | | PQGVTCGAATLSAERVRGDNKEYEYSVECQEDSACPAAEES |
| | | LPIEVMVDAVHKLAYENYTSSFFIRDIIKPDPPKNLQLKPL |
| | | KNSRQVEVSWEYPDTWSTPHSYFSLTFCVQVQGKSKREKKD |
| | | RVFTDKTSATVICRKNASISVRAQDRYYSSSWSEWASVPCS |
| hIL-12p40 | 58 | MCHQQLVISWFSLVFLASPLVAIWELKKDVYVVELDAYPDA |
| Variant W | | PGEMVVLTCDTPEEDGITWTLDQSSEVLGSGKTLTIQVKEF |
| (immature-signal | | GDAGQYTCHKGGEVLSHSLLLLHAKEDGIWSTDILKDQKEP |
| peptide underlined) | | KNKTFLRCEAKNYSGRFTCWWLTTISTDLTFSVKSSRGSSD |
| (W37A/K106A) | | PQGVTCGAATLSAERVRGDNKEYEYSVECQEDSACPAAEES |
| | | LPIEVMVDAVHKLKYENYTSSFFIRDIIKPDPPKNLQLKPL |
| | | KNSRQVEVSWEYPDTWSTPHSYFSLTFCVQVQGKSKREKKD |
| | | RVFTDKTSATVICRKNASISVRAQDRYYSSSWSEWASVPCS |
| hIL-12p40 | 59 | MCHQQLVISWESLVELASPLVAIWELKKDVYVVELDWYPDA |
| Variant X | | PGEMVVLTCDTPEEDGITWTLDQSSEVLGSGKTLTIQVKEA |
| (immature-signal | | GDAGQYTCHKGGEVLSHSLLLLHAKEDGIWSTDILKDQKEP |
| peptide underlined) | | KNKTFLRCEAKNYSGRFTCWWLTTISTDLTFSVKSSRGSSD |
| (F82A/K106A) | | PQGVTCGAATLSAERVRGDNKEYEYSVECQEDSACPAAEES |
| | | LPIEVMVDAVHKLKYENYTSSFFIRDIIKPDPPKNLQLKPL |
| | | KNSRQVEVSWEYPDTWSTPHSYFSLTFCVQVQGKSKREKKD |
| | | RVFTDKTSATVICRKNASISVRAQDRYYSSSWSEWASVPCS |
| hIL-12p40 | 60 | MCHQQLVISWESLVFLASPLVAIWELKKDVYVVELDWYPDA |
| Variant Y | | PGEMVVLTCDTPEEDGITWTLDQSSEVLGSGKTLTIQVKEA |
| (immature-signal | | GDAGQYTCHKGGEVLSHSLLLLHKKEDGIWSTDILKDQKEP |
| peptide underlined) | | KNKTFLRCEAKNYSGRFTCWWLTTISTDLTFSVKSSRGSSD |
| (F82A/K217A) | | PQGVTCGAATLSAERVRGDNKEYEYSVECQEDSACPAAEES |
| | | LPIEVMVDAVHALKYENYTSSFFIRDIIKPDPPKNLQLKPL |
| | | KNSRQVEVSWEYPDTWSTPHSYFSLTFCVQVQGKSKREKKD |
| | | RVFTDKTSATVICRKNASISVRAQDRYYSSSWSEWASVPCS |
| hIL-12p40 | 61 | MCHQQLVISWFSLVFLASPLVAIWELKKDVYVVELDWYPDA |
| Variant Z | | PGEMVVLTCDTPEEDGITWTLDQSSEVLGSGKTLTIQVKEA |
| (immature-signal | | GDAGQYTCHKGGEVLSHSLLLLHKKEDGIWSTDILKDQKEP |
| peptide underlined) | | KNKTFLRCEAKNYSGRFTCWWLTTISTDLTFSVKSSRGSSD |
| (F82A/K219A) | | PQGVTCGAATLSAERVRGDNKEYEYSVECQEDSACPAAEES |
| | | LPIEVMVDAVHKLAYENYTSSFFIRDIIKPDPPKNLQLKPL |
| | | KNSRQVEVSWEYPDTWSTPHSYFSLTFCVQVQGKSKREKKD |
| | | RVFTDKTSATVICRKNASISVRAQDRYYSSSWSEWASVPCS |
| hIL-12p40 | 62 | MCHQQLVISWESLVFLASPLVAIWELKKDVYVVELDWYPDA |
| Variant AA | | PGEMVVLTCDTPEEDGITWTLDQSSEVLGSGKTLTIQVKEF |
| (immature-signal | | GDAGQYTCHKGGEVLSHSLLLLHKKEDGIWSTDILKDQKEP |
| peptide underlined) | | KNKTFLRCEAKNYSGRFTCWWLTTISTDLTFSVKSSRGSSD |
| (K217A/K219A) | | PQGVTCGAATLSAERVRGDNKEYEYSVECQEDSACPAAEES |
| | | LPIEVMVDAVHALAYENYTSSFFIRDIIKPDPPKNLQLKPL |
| | | KNSRQVEVSWEYPDTWSTPHSYFSLTFCVQVQGKSKREKKD |
| | | RVFTDKTSATVICRKNASISVRAQDRYYSSSWSEWASVPCS |
| hIL-12p40 | 63 | MCHQQLVISWESLVELASPLVAIWELKKDVYVVELDWYPDA |
| Variant BB | | PGEMVVLTCDTPEEDGITWTLDQSSEVLGSGKTLTIQVKEF |
| (immature-signal | | GDAGQYTCHKGGEVLSHSLLLLHAKEDGIWSTDILKDQKEP |
| peptide underlined) | | KNKTFLRCEAKNYSGRFTCWWLTTISTDLTFSVKSSRGSSD |
| (K106A/K217A) | | PQGVTCGAATLSAERVRGDNKEYEYSVECQEDSACPAAEES |
| | | LPIEVMVDAVHALKYENYTSSFFIRDIIKPDPPKNLQLKPL |
| | | KNSRQVEVSWEYPDTWSTPHSYFSLTFCVQVQGKSKREKKD |
| | | RVFTDKTSATVICRKNASISVRAQDRYYSSSWSEWASVPCS |
| hIL-12p40 | 64 | MCHQQLVISWESLVFLASPLVAIWELKKDVYVVELDWYPDA |
| Variant CC | | PGEMVVLTCDTPEEDGITWTLDQSSEVLGSGKTLTIQVKEF |
| (immature-signal | | GDAGQYTCHKGGEVLSHSLLLLHAKEDGIWSTDILKDQKEP |
| peptide underlined) | | KNKTFLRCEAKNYSGRFTCWWLTTISTDLTFSVKSSRGSSD |
| (K106A/K219A) | | PQGVTCGAATLSAERVRGDNKEYEYSVECQEDSACPAAEES |
| | | LPIEVMVDAVHKLAYENYTSSFFIRDIIKPDPPKNLQLKPL |
| | | KNSRQVEVSWEYPDTWSTPHSYFSLTFCVQVQGKSKREKKD |
| | | RVFTDKTSATVICRKNASISVRAQDRYYSSSWSEWASVPCS |
| hIL-12p40 | 65 | MCHQQLVISWESLVFLASPLVAIWELKKDVYVVELDWYPDA |
| Variant H | | PGEMVVLTCDTPEEDGITWTLDQSSEVLGSGKTLTIQVKEF |
| (immature-signal | | GDAGQYTCHKGGEVLSHSLLLLHKKEDGIWSTDILKDQKEP |
| peptide underlined) | | KNKTFLRCEAKNYSGRFTCWWLTTISTDLTFSVKSSRGSSD |
| (K217 + L218 | | PQGVTCGAATLSAERVRGDNKEYEYSVECQEDSACPAAEES |
| substituted with I) | | LPIEVMVDAVHIKYENYTSSFFIRDIIKPDPPKNLQLKPLK |
| | | NSRQVEVSWEYPDTWSTPHSYFSLTFCVQVQGKSKREKKDR |
| | | VFTDKTSATVICRKNASISVRAQDRYYSSSWSEWASVPCS |

In some embodiments, the amino acid sequence of the hIL-12p40 polypeptide comprises or consists of the amino acid sequence of a polypeptide set forth in Table 5. In some embodiments, the amino acid sequence of the hIL-12p40 polypeptide comprises or consists of the amino acid sequence set forth in any one of SEQ ID NOS: 38-65. In some embodiments, the amino acid sequence of the hIL-12p40 polypeptide comprises or consists of the amino acid sequence set forth in SEQ ID NO: 38.

In some embodiments, the amino acid sequence of the hIL-12p40 polypeptide comprises or consists of a set of amino acid substitutions set forth in the amino acid sequence of any one polypeptide set forth in Table 5 (relative to the amino acid sequence of SEQ ID NO: 33); and other than the set of amino acid substitutions, the amino acid sequence of the hIL-12p40 polypeptide is at least 85%, 86%, 87%, 88%, 89%, 90%, 91%, 92%, 93%, 94%, 95%, 96%, 97%, 98%, 99%, or 100% identical to the amino acid sequence of the polypeptide set forth in Table 5.

In some embodiments, the amino acid sequence of the hIL-12p40 polypeptide comprises or consists of a set of amino acid substitutions set forth in the amino acid sequence of any one SEQ ID NOS: 38-65 (relative to the amino acid sequence of SEQ ID NO: 33); and other than the set of amino acid substitutions, the amino acid sequence of the hIL-12p40 polypeptide is at least 85%, 86%, 87%, 88%, 89%, 90%, 91%, 92%, 93%, 94%, 95%, 96%, 97%, 98%, 99%, or 100% identical to the amino acid sequence of set forth in the any one of SEQ ID NOS: 38-65.

In some embodiments, the amino acid sequence of the hIL-12p40 polypeptide comprises or consists of a set of amino acid substitutions set forth in the amino acid sequence of SEQ ID NOS: 38 (relative to the amino acid sequence of SEQ ID NO: 33); and other than the set of amino acid substitutions, the amino acid sequence of the hIL-12p40 polypeptide is at least 85%, 86%, 87%, 88%, 89%, 90%, 91%, 92%, 93%, 94%, 95%, 96%, 97%, 98%, 99%, or 100% identical to the amino acid sequence of SEQ ID NO: 38.

5.2.2 Potency & Affinity of hIL-12p40 Proteins & Polypeptides

In some embodiments, when combined with a hIL-12p35 protein the hIL-12p40 protein mediates a lower increase in the level of STAT4 in cells expressing the hIL-12R on the surface relative to the increase in STAT4 mediated by a suitable control (e.g., a reference hIL-12p40 protein (e.g., SEQ ID NO: 33)). In some embodiments, when combined with a hIL-12p35 protein the hIL-12p40 protein mediates a lower increase in the level of phosphorylated STAT4 (pSTAT4) in cells expressing the hIL-12R on the surface relative to the increase in pSTAT4 mediated by a suitable control (e.g., a reference hIL-12p40 protein (e.g., SEQ ID NO: 33)).

In some embodiments, when combined with a hIL-12p35 protein the hIL-12p40 protein mediates about a 0.5-fold, 1-fold, 2-fold, 5-fold, 10-fold, 100-fold, or 1000-fold lower increase in the level of phosphorylated STAT4 (pSTAT4) in cells expressing the hIL-12R on the surface relative to the increase in pSTAT4 mediated by a suitable control (e.g., a reference hIL-12p40 protein (e.g., SEQ ID NO: 33)). In some embodiments, when combined with a hIL-12p35 protein the hIL-12p40 protein mediates at least about a 0.5-fold, 1-fold, 2-fold, 5-fold, 10-fold, 100-fold, or 1000-fold lower increase in the level of phosphorylated STAT4 (pSTAT4) in cells expressing the hIL-12R on the surface relative to the increase in pSTAT4 mediated by a suitable control (e.g., a reference hIL-12p40 protein (e.g., SEQ ID NO: 33)). In some embodiments, when combined with a hIL-12p35 protein the hIL-12p40 protein mediates from about a 0.5-1000 fold, 0.5-100 fold, 0.5-10 fold, 0.5-5 fold, 0.5-2 fold, 1-1000 fold, 1-100 fold, 1-10 fold, 1-5 fold, 1-2 fold, 10-1000 fold, or 100-1000 fold lower increase in the level of phosphorylated STAT4 (pSTAT4) in cells expressing the hIL-12R on the surface relative to the increase in pSTAT4 mediated by a suitable control (e.g., a reference hIL-12p40 protein (e.g., SEQ ID NO: 33)).

Assays suitable to measure the EC50 of a hIL-12p40 polypeptide described herein are standard and known to the person of ordinary skill in the art. For example, the EC50 can be determined by constructing a dose-response curve and examining the effect of different concentrations of the hIL-12p40 protein or polypeptide in inducing activity in a particular functional assay (e.g., STAT4 signaling, STAT4 phosphorylation, STAT4 inducible SEAP expression (see, e.g., Example 4)). § 6.4 describes an exemplary method of determining the EC50 of a hIL-12p40 polypeptide or protein described herein (including hIL-12 fusion proteins described herein) utilizing the hIL-12 HEKBlue reporter cell line (InvivoGen #hkb-IL12). The hIL-12 HEKBlue reporter cell line expresses the hIL-12Rβ1 and hIL-12Rβ2 subunits, human STAT4, and a STAT4-inducible SEAP (secreted embryonic alkaline phosphatase) reporter. Thereby, binding of a protein to the hIL-12R triggers JAK2/STAT4 signaling and the subsequent production of SEAP, which can be quantified using standard methods known in the art. Additionally for example, the level of phosphorylated STAT4 can be assessed by contacting cells expressing the hIL-12R with one or more concentration of hIL-12p40 protein or polypeptide described herein, lysing the cells, and assessing the level of phosphorylated STAT4, e.g., by ELISA, Western blot, FRET-based assay or chemiluminescent assay (e.g., ELISA-based assay). The cells in the cell-based assay may be cells, such as HEK293 cells, which recombinantly express the hIL-12R and/or human STAT4; or cells that naturally express hIL-12R and human STAT4.

In some embodiments, when combined with a hIL-12p35 protein the hIL-12p40 protein mediates a lower increase the level of interferon gamma (IFN-γ) produced by expressing the hIL-12R on the surface relative to the increase in the level of IFN-γ produced in the presence of a suitable control (e.g., a reference hIL-12p40 protein (e.g., SEQ ID NO: 33)).

In some embodiments, when combined with a hIL-12p35 protein the hIL-12p40 protein mediates a 0.5-fold, 1-fold, 2-fold, 5-fold, 10-fold, 100-fold, or 1000-fold lower increase in the level of IFN-γ produced by cell expressing the hIL-12R on the surface, relative to the increase in the level of IFN-γ produced in the presence of a suitable control (e.g., a reference hIL-12p40 protein (e.g., a reference hIL-12p40 protein (e.g., SEQ ID NO: 33)).

In some embodiments, when combined with a hIL-12p35 protein the hIL-12p40 protein mediates at least about 0.5-fold, 1-fold, 2-fold, 5-fold, 10-fold, 100-fold, or 1000-fold lower increase in the level of IFN-γ produced by cells expressing the hIL-12R on the surface, relative to the increase in the level of IFN-γ produced in the presence of a suitable control (e.g., a reference hIL-12p40 protein (e.g., SEQ ID NO: 33)).

In some embodiments, when combined with a hIL-12p35 protein the hIL-12p40 protein mediates from about a 0.5-1000 fold, 0.5-100 fold, 0.5-10 fold, 0.5-5 fold, 0.5-2 fold, 1-1000 fold, 1-100 fold, 1-10 fold, 1-5 fold, 1-2 fold, 10-1000 fold, or 100-1000 fold lower increase in the level of IFN-γ produced by cells expressing the hIL-12R on the surface, relative to the increase in the level of IFN-γ produced in the presence of a suitable control (e.g., a reference hIL-12p40 protein (e.g., SEQ ID NO: 33)).

Assays suitable to measure the level of a protein (e.g., IFN-γ) produced from cultured cells are standard and known to the person of ordinary skill in the art. For example, the level of IFN-γ can be determined using an enzyme linked immunosorbent assay (ELISA) (see, e.g., Example 5)). § 6.5 describes an exemplary method of determining the level of IFN-γ produced from cultured cells treated with an hIL-12p40 protein described herein or a reference hIL-12p40 protein.

In some embodiments, the hIL-12p40 protein binds to hIL-12Rβ1 with lower affinity relative to that of a reference hIL-12p40 protein (e.g., a reference hIL-12p40 protein (e.g., SEQ ID NO: 33)). Binding affinity can be measured by standard assays known in the art. For example, binding affinity can be measured by surface plasmon resonance (SPR) (e.g., BIAcore®-based assay), a common method known in the art (see, e.g., Wilson, Science 295:2103, 2002; Wolff et al., Cancer Res. 55:2560, 1993; and U.S. Pat. Nos. 5,283,173, 5,468,614, the full contents of each of which are incorporated herein by reference for all purposes). SPR measures changes in the concentration of molecules at a sensor surface as molecules bind to or dissociate from the surface. The change in the SPR signal is directly proportional to the change in mass concentration close to the surface, thereby allowing measurement of binding kinetics between two molecules (e.g., proteins). The dissociation constant for the complex can be determined by monitoring changes in the refractive index with respect to time as buffer is passed over the chip.

Other suitable assays for measuring the binding of one protein to another (e.g., binding of a protein described herein to hIL-12Rβ1) include, for example, immunoassays such as enzyme linked immunosorbent assays (ELISA) and radioimmunoassays (RIA), or determination of binding by monitoring the change in the spectroscopic or optical properties of the proteins through fluorescence, UV absorption, circular dichroism, or nuclear magnetic resonance (NMR). Other exemplary assays include, but are not limited to, Western blot, analytical ultracentrifugation, spectroscopy, flow cytometry, sequencing and other methods for detection of binding of proteins.

5.2.3 hIL-12p35 Proteins & Polypeptides

In one aspect, provided herein are hIL-12p35 polypeptides with attenuated activity compared to a reference hIL-12p35 polypeptide (e.g., SEQ ID NO: 31). As described herein, any of the hIL-12p35 polypeptides described herein may be isolated and/or recombinant. In some embodiments, the hIL-12p35 polypeptide specifically binds the hIL-12R. In some embodiments, the hIL-12p35 polypeptide specifically binds the hIL-12Rβ1 (see also, § 5.2.3). In some embodiments, the hIL-12p35 polypeptide specifically binds the hIL-12Rβ2 (see also, § 5.2.3).

As set for the above, for the purposes of the instant disclosure, the numbering of all amino acids (and e.g., amino acid substitutions) of hIL-12p35 polypeptides described herein is set out relative to the amino acid sequence of the immature form of hIL-12p35 (i.e., SEQ ID NO: 30), that contains the native signal peptide. The use of the immature form of hIL-12p35 to designate amino acid numbers (e.g., Y189) is for consistency only and does not limit the scope of embodiments utilizing this numbering scheme to polypeptides that contain the signal peptide of hIL-12p35. For example, a hIL-12p35 polypeptide described herein as comprising the amino acid sequence of SEQ ID NO: 31 with a Y189A amino acid substitution does not require the signal peptide of hIL-12p35, although the numbering of amino acid position Y189 is based on the immature form of the protein. It common in the art to utilize the mature form of a protein to produce variants and fusion proteins.

A person of ordinary skill in art can easily determine the amino acid position in the mature form of hIL-12p35 (SEQ ID NO: 31) based on the amino acid numbering relative to the immature form of hIL-12p35. As set forth above, amino acids 1-22 of the immature form of the hIL-12p35 protein are the signal sequence. Therefore, an amino acid position of a particular amino acid in the mature form of the hIL-12p35 protein can be determined from the amino acid position of the particular amino acid designated relative to the immature form of hIL-12p35 by subtracting 22. For example, the amino acid position Y189 (numbering relative to SEQ ID NO: 30) would correspond to amino acid position Y167 in the mature form of the protein (SEQ ID NO: 31).

In some embodiments, the amino acid sequence of the hIL-12p35 polypeptide comprises or consists of an amino acid sequence (a) at least 85%, 86%, 87%, 88%, 89%, 90%, 91%, 92%, 93%, 94%, 95%, 96%, 97%, 98%, or 99% identical to the amino acid sequence of SEQ ID NO: 31; and (b) comprises or consists of an amino acid modification (e.g., substitution, addition, deletion (e.g., substitution)) at one or more of the following amino acid positions E60, F61, P63, K150, F188, Y189A, amino acid numbering relative to the amino acid sequence of SEQ ID NO: 30.

In some embodiments, the amino acid sequence of the hIL-12p35 polypeptide comprises or consists of an amino acid sequence (a) at least 85%, 86%, 87%, 88%, 89%, 90%, 91%, 92%, 93%, 94%, 95%, 96%, 97%, 98%, or 99% identical to the amino acid sequence of SEQ ID NO: 31; and (b) comprises or consists of an amino acid modification (e.g., substitution, addition, deletion (e.g., substitution)) at each of the following amino acid positions (i) F188; (ii) Y189; (iii) F188 and Y189; or (iv) E60, F61, P63, K150, and F188, amino acid numbering relative to the amino acid sequence of SEQ ID NO: 30.

In some embodiments, the amino acid sequence of the hIL-12p35 polypeptide comprises or consists of an amino acid sequence (a) at least 85%, 86%, 87%, 88%, 89%, 90%, 91%, 92%, 93%, 94%, 95%, 96%, 97%, 98%, or 99% identical to the amino acid sequence of SEQ ID NO: 31; and (b) comprises or consists of one or more of the following amino acid substitutions: E60K, F61H, P63S, K150H, F188P, F188A, and/or Y189A, amino acid numbering relative to the amino acid sequence of SEQ ID NO: 30.

In some embodiments, the amino acid sequence of the hIL-12p35 polypeptide comprises or consists of an amino acid sequence (a) at least 85%, 86%, 87%, 88%, 89%, 90%, 91%, 92%, 93%, 94%, 95%, 96%, 97%, 98%, or 99% identical to the amino acid sequence of SEQ ID NO: 31; and (b) comprises or consists of each of the following amino acid substitutions: (i) F188A; (ii) Y189A; (iii) F188A and Y189A; or (iv) E60K, F61H, P63S, K150H, and F188P, amino acid numbering relative to the amino acid sequence of SEQ ID NO: 30.

In some embodiments, the amino acid sequence of the hIL-12p35 polypeptide comprises or consists of an amino acid sequence (a) at least 85%, 86%, 87%, 88%, 89%, 90%, 91%, 92%, 93%, 94%, 95%, 96%, 97%, 98%, or 99% identical to the amino acid sequence of SEQ ID NO: 31; and (b) comprises or consists of a deletion of amino acids A55-K92, N50-K92, M51-K92, L52-K92, Q53-K92, K54-K92, N50-N93, M51-N93, L52-N93, Q53-N93, K54-N93, N50-E94, M51-E94, L52-E94, Q53-E94, K54-E94, N50-S95, M51-S95, L52-S95, Q53-S95, K54-S95, N50-C96, M51-C96, L52-C96, Q53-C96, K54-C96, N50-L97, M51-L97, L52-L97, Q53-L97, K54-L97, N50-P87, M51-P87, L52-P87, Q53-P87, K54-P87, N50-L88, M51-L88, L52-L88, Q53-L88, K54-L88, N50-E89, M51-E89, L52-E89, Q53-E89, K54-E89, N50-L90, M51-L90, L52-L90, Q53-L90, K54-L90, N50-T91, M51-T91, L52-T91, Q53-T91, K54-T91, R56-K92, Q57-K92, T58-K92, L59-K92, E60-K92 A55-N93, R56-N93, Q57-N93, T58-N93, L59-N93, E60-N93, A55-E94, R56-E94, Q57-E94, T58-E94, L59-E94, E60-E94, A55-S95, R56-S95, Q57-S95, T58-S95, L59-S95, E60-S95, A55-C96, R56-C96, Q57-C96, T58-C96, L59-C96, E60-C96, A55-L97, R56-L97, Q57-L97, T58-L97, L59-L97, E60-L97, A55-P87, R56-P87, Q57-P87, T58-P87, L59-P87, E60-P87, A55-L88, R56-L88, Q57-L88, T58-L88, L59-L88, E60-L88, A55-E89, R56-E89, Q57-E89, T58-E89, L59-E89, E60-E89, A55-L90, R56-L90, Q57-L90, T58-L90, L59-L90, E60-L90, A55-T91, R56-T91, Q57-T91, T58-T91, L59-T91, or E60-T91, amino acid numbering relative to the amino acid sequence of SEQ ID NO: 30.

In some embodiments, the amino acid sequence of the hIL-12p35 polypeptide comprises or consists of an amino acid sequence (a) at least 85%, 86%, 87%, 88%, 89%, 90%, 91%, 92%, 93%, 94%, 95%, 96%, 97%, 98%, or 99% identical to the amino acid sequence of SEQ ID NO: 31; and (b) comprises or consists of a deletion of amino acids A55-K92, amino acid numbering relative to the amino acid sequence of SEQ ID NO: 30.

In some embodiments, the amino acid sequence of the hIL-12p35 polypeptide comprises or consists of a deletion of amino acids A55-K92, N50-K92, M51-K92, L52-K92, Q53-K92, K54-K92, N50-N93, M51-N93, L52-N93, Q53-N93, K54-N93, N50-E94, M51-E94, L52-E94, Q53-E94, K54-E94, N50-S95, M51-S95, L52-S95, Q53-S95, K54-S95, N50-C96, M51-C96, L52-C96, Q53-C96, K54-C96, N50-L97, M51-L97, L52-L97, Q53-L97, K54-L97, N50-P87, M51-P87, L52-P87, Q53-P87, K54-P87, N50-L88, M51-L88, L52-L88, Q53-L88, K54-L88, N50-E89, M51-E89, L52-E89, Q53-E89, K54-E89, N50-L90, M51-L90, L52-L90, Q53-L90, K54-L90, N50-T91, M51-T91, L52-T91, Q53-T91, K54-T91, R56-K92, Q57-K92, T58-K92, L59-K92, E60-K92 A55-N93, R56-N93, Q57-N93, T58-N93, L59-N93, E60-N93, A55-E94, R56-E94, Q57-E94, T58-E94, L59-E94, E60-E94, A55-S95, R56-S95, Q57-S95, T58-S95, L59-S95, E60-S95, A55-C96, R56-C96, Q57-C96, T58-C96, L59-C96, E60-C96, A55-L97, R56-L97, Q57-L97, T58-L97, L59-L97, E60-L97, A55-P87, R56-P87, Q57-P87, T58-P87, L59-P87, E60-P87, A55-L88, R56-L88, Q57-L88, T58-L88, L59-L88, E60-L88, A55-E89, R56-E89, Q57-E89, T58-E89, L59-E89, E60-E89, A55-L90, R56-L90, Q57-L90, T58-L90, L59-L90, E60-L90, A55-T91, R56-T91, Q57-T91, T58-T91, L59-T91, or E60-T91 (amino acid numbering relative to the amino acid sequence of SEQ ID NO: 30), and other than the deletion of amino acids A55-K92, N50-K92, M51-K92, L52-K92, Q53-K92, K54-K92, N50-N93, M51-N93, L52-N93, Q53-N93, K54-N93, N50-E94, M51-E94, L52-E94, Q53-E94, K54-E94, N50-S95, M51-S95, L52-S95, Q53-S95, K54-S95, N50-C96, M51-C96, L52-C96, Q53-C96, K54-C96, N50-L97, M51-L97, L52-L97, Q53-L97, K54-L97, N50-P87, M51-P87, L52-P87, Q53-P87, K54-P87, N50-L88, M51-L88, L52-L88, Q53-L88, K54-L88, N50-E89, M51-E89, L52-E89, Q53-E89, K54-E89, N50-L90, M51-L90, L52-L90, Q53-L90, K54-L90, N50-T91, M51-T91, L52-T91, Q53-T91, K54-T91, R56-K92, Q57-K92, T58-K92, L59-K92, E60-K92 A55-N93, R56-N93, Q57-N93, T58-N93, L59-N93, E60-N93, A55-E94, R56-E94, Q57-E94, T58-E94, L59-E94, E60-E94, A55-S95, R56-S95, Q57-S95, T58-S95, L59-S95, E60-S95, A55-C96, R56-C96, Q57-C96, T58-C96, L59-C96, E60-C96, A55-L97, R56-L97, Q57-L97, T58-L97, L59-L97, E60-L97, A55-P87, R56-P87, Q57-P87, T58-P87, L59-P87, E60-P87, A55-L88, R56-L88, Q57-L88, T58-L88, L59-L88, E60-L88, A55-E89, R56-E89, Q57-E89, T58-E89, L59-E89, E60-E89, A55-L90, R56-L90, Q57-L90, T58-L90, L59-L90, E60-L90, A55-T91, R56-T91, Q57-T91, T58-T91, L59-T91, or E60-T91 the amino acid sequence of the polypeptide is at least 85%, 86%, 87%, 88%, 89%, 90%, 91%, 92%, 93%, 94%, 95%, 96%, 97%, 98%, or 99% identical to the amino acid sequence of SEQ ID NO: 31.

In some embodiments, the amino acid sequence of the hIL-12p35 polypeptide comprises or consists of a deletion of amino acids A55-K92 (amino acid numbering relative to the amino acid sequence of SEQ ID NO: 30), and other than the deletion of amino acids A55-K92 the amino acid sequence of the polypeptide is at least 85%, 86%, 87%, 88%, 89%, 90%, 91%, 92%, 93%, 94%, 95%, 96%, 97%, 98%, or 99% identical to the amino acid sequence of SEQ ID NO: 31

In some embodiments, the amino acid sequence of the hIL-12p35 polypeptide comprises or consists of an amino acid modification (e.g., substitution, addition, or deletion) at one or more of amino acid positions E60, F61, P63, K150, F188, or Y189, amino acid numbering relative to the amino acid sequence set forth SEQ ID NO: 30. In some embodiments, the amino acid sequence of the hIL-12p35 polypeptide comprises or consists of an amino acid modification (e.g., substitution, addition, or deletion) at 1, 2, 3, 4, 5, 6, or 7 of the following amino acid positions E60, F61, P63, K150, F188, or Y189, amino acid numbering relative to the amino acid sequence set forth SEQ ID NO: 30.

In some embodiments, the amino acid sequence of the hIL-12p35 polypeptide comprises or consists of an amino acid modification (e.g., substitution, addition, or deletion) at one or more of amino acid positions E60, F61, P63, K150, and F188, amino acid numbering relative to the amino acid sequence set forth SEQ ID NO: 30. In some embodiments, the amino acid sequence of the hIL-12p35 polypeptide comprises or consists of an amino acid modification (e.g., substitution, addition, or deletion) at one or more of amino acid positions F188 and Y189, amino acid numbering relative to the amino acid sequence set forth SEQ ID NO: 30. In some embodiments, the amino acid sequence of the hIL-12p35 polypeptide comprises or consists of an amino acid modification (e.g., substitution, addition, or deletion) at amino acid position F188, amino acid numbering relative to the amino acid sequence set forth SEQ ID NO: 30. In some embodiments, the amino acid sequence of the hIL-12p35 polypeptide comprises or consists of an amino acid modification (e.g., substitution, addition, or deletion) at amino acid position Y189A, amino acid numbering relative to the amino acid sequence set forth SEQ ID NO: 30.

In some embodiments, the amino acid sequence of the hIL-12p35 polypeptide comprises or consists of one or more of the following amino acid substitutions: E60K, F61H, P63S, K150H, F188P, F188A, or Y189A amino acid numbering relative to the amino acid sequence set forth SEQ ID NO: 30. In some embodiments, the amino acid sequence of the hIL-12p35 polypeptide comprises or consists of the following amino acid substitutions: E60K, F61H, P63S, K150H, and F188P, amino acid numbering relative to the amino acid sequence set forth SEQ ID NO: 30. In some embodiments, the amino acid sequence of the hIL-12p35 polypeptide comprises or consists of the following amino acid substitutions: F188A and Y189A, amino acid numbering relative to the amino acid sequence set forth SEQ ID NO: 30. In some embodiments, the amino acid sequence of the hIL-12p35 polypeptide comprises or consists of the following amino acid substitution F188A, amino acid numbering relative to the amino acid sequence set forth SEQ ID NO: 30. In some embodiments, the amino acid sequence of the hIL-12p35 polypeptide comprises or consists of the following amino acid substitution Y189A, amino acid numbering relative to the amino acid sequence set forth SEQ ID NO: 30.

In some embodiments, the amino acid sequence of the hIL12-p35 polypeptide comprises a deletion of amino acid residues 50-95, 50-94, 50-93, 50-92, 50-91, 50-90, 50-89, 51-95, 51-94, 51-93, 51-92, 51-91, 51-90, 51-89, 52-95, 52-94, 52-93, 52-92, 52-91, 52-90, 52-89, 53-95, 53-94, 53-93, 53-92, 53-91, 53-90, 53-89, 54-95, 54-94, 54-93, 54-92, 54-91, 54-90, 54-89, 55-95, 55-94, 55-93, 55-92, 55-91, 55-90, or 55-89; amino acid numbering relative to the amino acid sequence set forth SEQ ID NO: 30. In some embodiments, the amino acid sequence of the hIL12-p35 polypeptide comprises a deletion of amino acid residues A55-K92; amino acid numbering relative to the amino acid sequence set forth SEQ ID NO: 30.

The amino acid sequence of exemplary hIL-12p35 polypeptides is provided in Table 6.

TABLE 6
Amino Acid Sequences of Exemplary hIL-12p35 Polypeptides

| | | SEQ ID |
|---|---|---|
| Description | Amino Acid Sequence | NO |
| hIL-12p35-Variant A | RNLPVATPDPGMFPCLHHSQNLLRAVSNMLQKNESCLNSRETSF | 110 |
| (ΔA55-K92) | ITNGSCLASRKTSFMMALCLSSIYEDLKMYQVEFKTMNAKLLMD | |
| | PKRQIFLDQNMLAVIDELMQALNENSETVPQKSSLEEPDFYKTK | |
| | IKLCILLHAFRIRAVTIDRVMSYLNAS | |
| hIL-12p35-Variant B | RNLPVATPDPGMFPCLHHSQNLLRAVSNMLQKARQTLKHYSCTS | 111 |
| (E60K, F61H, P63S, | EEIDHEDITKDKTSTVEACLPLELTKNESCLNSRETSFITNGSC | |
| K150H, F188P) | LASRKTSEMMALCLSSIYEDLKMYQVEFKTMNAKLLMDPHRQIF | |
| | LDQNMLAVIDELMQALNENSETVPQKSSLEEPDPYKTKIKLCIL | |
| | LHAFRIRAVTIDRVMSYLNAS | |
| hIL-12p35-Variant C | RNLPVATPDPGMFPCLHHSQNLLRAVSNMLQKARQTLEFYPCTS | 112 |
| (F188A, Y189A) | EEIDHEDITKDKTSTVEACLPLELTKNESCLNSRETSFITNGSC | |
| | LASRKTSFMMALCLSSIYEDLKMYQVEFKTMNAKLLMDPKRQIF | |
| | LDQNMLAVIDELMQALNENSETVPQKSSLEEPDAAKTKIKLCIL | |
| | LHAFRIRAVTIDRVMSYLNAS | |
| hIL-12p35-Variant D | RNLPVATPDPGMFPCLHHSQNLLRAVSNMLQKARQTLEFYPCTS | 113 |
| (F188A) | EEIDHEDITKDKTSTVEACLPLELTKNESCLNSRETSFITNGSC | |
| | LASRKTSFMMALCLSSIYEDLKMYQVEFKTMNAKLLMDPKRQIF | |
| | LDQNMLAVIDELMQALNENSETVPQKSSLEEPDAYKTKIKLCIL | |
| | LHAFRIRAVTIDRVMSYLNAS | |
| hIL-12p35-Variant E | RNLPVATPDPGMFPCLHHSQNLLRAVSNMLQKARQTLEFYPCTS | 114 |
| (Y189A) | EEIDHEDITKDKTSTVEACLPLELTKNESCLNSRETSFITNGSC | |
| | LASRKTSEMMALCLSSIYEDLKMYQVEFKTMNAKLLMDPKRQIF | |
| | LDQNMLAVIDELMQALNENSETVPQKSSLEEPDFAKTKIKLCIL | |
| | LHAFRIRAVTIDRVMSYLNAS | |

In some embodiments, the amino acid sequence of the hIL-12p35 polypeptide comprises an amino acid sequence at least 85%, 86%, 87%, 88%, 89%, 90%, 91%, 92%, 93%, 94%, 95%, 96%, 97%, 98%, 99%, or 100% identical to the amino acid sequence of any one of the polypeptides set forth in Table 6.

In some embodiments, the amino acid sequence of the hIL-12p35 polypeptide comprises an amino acid sequence at least 85%, 86%, 87%, 88%, 89%, 90%, 91%, 92%, 93%, 94%, 95%, 96%, 97%, 98%, 99%, or 100% identical to the amino acid sequence of any one of SEQ ID NOS: 31 or 110-114.

In some embodiments, the amino acid sequence of the hIL-12p35 polypeptide comprises or consists of a set of amino acid modifications (e.g., substitutions, deletions) set forth in the amino acid sequence of any one polypeptide set forth in Table 6 (relative to the amino acid sequence of SEQ ID NO: 31); and other than the set of amino acid modifications (e.g., substitutions, deletions), the amino acid sequence of the hIL-12p35 polypeptide is at least 85%, 86%, 87%, 88%, 89%, 90%, 91%, 92%, 93%, 94%, 95%, 96%, 97%, 98%, 99%, or 100% identical to the amino acid sequence of the polypeptide set forth in Table 6.

In some embodiments, the amino acid sequence of the hIL-12p35 polypeptide comprises or consists of a set of amino acid modifications (e.g., substitutions, deletions) set forth in the amino acid sequence of any one SEQ ID NOS: 110-114 (relative to the amino acid sequence of SEQ ID NO: 31); and other than the set of amino acid modifications (e.g., substitutions, deletions), the amino acid sequence of the hIL-12p35 polypeptide is at least 85%, 86%, 87%, 88%, 89%, 90%, 91%, 92%, 93%, 94%, 95%, 96%, 97%, 98%, 99%, or 100% identical to the amino acid sequence of set forth in the any one of SEQ ID NOS: 110-114.

5.2.4 Potency & Affinity of hIL-12p35 Proteins & Polypeptides

In some embodiments, when combined with a hIL-12p40 protein the hIL-12p35 protein mediates a lower increase in the level of STAT4 in cells expressing the hIL-12R on the surface relative to the increase in STAT4 mediated by a suitable control (e.g., a reference hIL-12p35 protein (e.g., SEQ ID NO: 31)). In some embodiments, when combined with a hIL-12p40 protein the hIL-12p35 protein mediates a lower increase in the level of phosphorylated STAT4 (pSTAT4) in cells expressing the hIL-12R on the surface relative to the increase in pSTAT4 mediated by a suitable control (e.g., a reference hIL-12p35 protein (e.g., SEQ ID NO: 30)).

In some embodiments, when combined with a hIL-12p40 protein the hIL-12p35 protein mediates about a 0.5-fold, 1-fold, 2-fold, 5-fold, 10-fold, 100-fold, or 1000-fold lower increase in the level of phosphorylated STAT4 (pSTAT4) in cells expressing the hIL-12R on the surface relative to the increase in pSTAT4 mediated by a suitable control (e.g., a reference hIL-12p35 protein (e.g., SEQ ID NO: 30)). In some embodiments, when combined with a hIL-12p40 protein the hIL-12p35 protein mediates at least about a 0.5-fold, 1-fold, 2-fold, 5-fold, 10-fold, 100-fold, or 1000-fold lower increase in the level of phosphorylated STAT4 (pSTAT4) in cells expressing the hIL-12R on the surface relative to the increase in pSTAT4 mediated by a suitable control (e.g., a reference hIL-12p35 protein (e.g., SEQ ID NO: 30)). In some embodiments, when combined with a hIL-12p35 protein the hIL-12p40 protein mediates from about a 0.5-1000 fold, 0.5-100 fold, 0.5-10 fold, 0.5-5 fold, 0.5-2 fold, 1-1000 fold, 1-100 fold, 1-10 fold, 1-5 fold, 1-2 fold, 10-1000 fold, or 100-1000 fold lower increase in the level of phosphorylated STAT4 (pSTAT4) in cells expressing the hIL-12R on the surface relative to the increase in pSTAT4 mediated by a suitable control (e.g., a reference hIL-12p35 protein (e.g., SEQ ID NO: 30)).

Assays suitable to measure the EC50 of a hIL-12p35 polypeptide described herein are standard and known to the person of ordinary skill in the art. For example, the EC50 can be determined by constructing a dose-response curve and examining the effect of different concentrations of the hIL-12p35 protein or polypeptide in inducing activity in a particular functional assay (e.g., STAT4 signaling, STAT4 phosphorylation, STAT4 inducible SEAP expression (see, e.g., Example 21)). § 6.4 describes an exemplary method of determining the EC50 of a hIL-12p35 polypeptide or protein described herein (including hIL-12 fusion proteins described herein) utilizing the hIL-12 HEKBlue reporter cell line (InvivoGen #hkb-IL12). The hIL-12 HEKBlue reporter cell line expresses the hIL-12Rβ1 and hIL-12Rβ2 subunits, human STAT4, and a STAT4-inducible SEAP (secreted embryonic alkaline phosphatase) reporter. Thereby, binding of a protein to the hIL-12R triggers JAK2/STAT4 signaling and the subsequent production of SEAP, which can be quantified using standard methods known in the art. Additionally for example, the level of phosphorylated STAT4 can be assessed by contacting cells expressing the hIL-12R with one or more concentration of hIL-12p35 protein or polypeptide described herein, lysing the cells, and assessing the level of phosphorylated STAT4, e.g., by ELISA, Western blot, FRET-based assay or chemiluminescent assay (e.g., ELISA-based assay). The cells in the cell-based assay may be cells, such as HEK293 cells, which recombinantly express the hIL-12R and/or human STAT4; or cells that naturally express hIL-12R and human STAT4.

In some embodiments, when combined with a hIL-12p40 protein the hIL-12p35 protein mediates a lower increase the level of interferon gamma (IFN-γ) produced by expressing the hIL-12R on the surface relative to the increase in the level of IFN-γ produced in the presence of a suitable control (e.g., a reference hIL-12p35 protein (e.g., SEQ ID NO: 30)).

In some embodiments, when combined with a hIL-12p40 protein the hIL-12p35 protein mediates a 0.5-fold, 1-fold, 2-fold, 5-fold, 10-fold, 100-fold, or 1000-fold lower increase in the level of IFN-γ produced by cell expressing the hIL-12R on the surface, relative to the increase in the level of IFN-γ produced in the presence of a suitable control (e.g., a reference hIL-12p35 protein (e.g., a reference hIL-12p35 protein (e.g., SEQ ID NO: 30)).

In some embodiments, when combined with a hIL-12p40 protein the hIL-12p35 protein mediates at least about 0.5-fold, 1-fold, 2-fold, 5-fold, 10-fold, 100-fold, or 1000-fold lower increase in the level of IFN-γ produced by cells expressing the hIL-12R on the surface, relative to the increase in the level of IFN-γ produced in the presence of a suitable control (e.g., a reference hIL-12p35 protein (e.g., SEQ ID NO: 33)).

In some embodiments, when combined with a hIL-12p40 protein the hIL-12p35 protein mediates from about a 0.5-1000 fold, 0.5-100 fold, 0.5-10 fold, 0.5-5 fold, 0.5-2 fold, 1-1000 fold, 1-100 fold, 1-10 fold, 1-5 fold, 1-2 fold, 10-1000 fold, or 100-1000 fold lower increase in the level of IFN-γ produced by cells expressing the hIL-12R on the surface, relative to the increase in the level of IFN-γ produced in the presence of a suitable control (e.g., a reference hIL-12p35 protein (e.g., SEQ ID NO: 30)).

Assays suitable to measure the level of a protein (e.g., IFN-γ) produced from cultured cells are standard and known to the person of ordinary skill in the art. For example, the level of IFN-γ can be determined using an enzyme linked immunosorbent assay (ELISA) (see, e.g., Examples 22-23)). § 6.5 describes an exemplary method of determining the level of IFN-γ produced from cultured cells treated with an hIL-12p40 protein described herein or a reference hIL-12p40 protein.

In some embodiments, the hIL-12p35 protein binds to hIL-12Rβ1 with lower affinity relative to that of a reference hIL-12p35 protein (e.g., a reference hIL-12p35 protein (e.g., SEQ ID NO: 30)). Binding affinity can be measured by standard assays known in the art, see, e.g., § 5.2.2.

5.2.5 hIL-12 Single Chain Polypeptides & Proteins

In one aspect, provided herein are schIL-12 polypeptides that comprise a hIL-12p40 polypeptide described herein in § 5.2.1 and/or a hIL-12p35 polypeptide described herein in § 5.2.3. In some embodiments, wherein the schIL-12 polypeptide comprises a hIL-12p40 polypeptide described herein in § 5.2.1, the hIL-12p35 polypeptide is a hIL-12p35 polypeptide described herein in § 5.3.1.2. In some embodiments, wherein the schIL-12 polypeptide comprises a hIL-12p40 polypeptide described herein in § 5.2.1, the hIL-12p35 polypeptide is a hIL-12p35 polypeptide described herein in § 5.2.3.

In some embodiments, wherein the schIL-12 polypeptide comprises a hIL-12p35 polypeptide described herein in § 5.2.3, the hIL-12p40 polypeptide is a hIL-12p40 polypeptide described herein in § 5.2.1. In some embodiments, wherein the schIL-12 polypeptide comprises a hIL-12p35 polypeptide described herein in § 5.2.3, the hIL-12p40 polypeptide is a hIL-12p35 polypeptide described herein in § 5.3.1.1.

In some embodiments, the schIL-12 comprises a hIL-12p35 polypeptide directly fused to a hIL-12p40 polypeptide. In some embodiments, the schIL-12 polypeptide comprises from N- to C-terminus a hIL-12p35 polypeptide, an optional peptide linker, and a hIL-12p40 polypeptide. In some embodiments, the schIL-12 polypeptide comprises from N- to C-terminus a hIL-12p40 polypeptide, an optional peptide linker, and a hIL-12p35 polypeptide.

In some embodiments, the schIL-12 polypeptide comprises a hIL-12p35 polypeptide directly operably connected via a peptide bond. In some embodiments, the schIL-12 polypeptide comprises a hIL-12p35 polypeptide indirectly fused to a hIL-12p40 polypeptide via a peptide linker. In some embodiments, the schIL-12 polypeptide comprises from N- to C-terminus a hIL-12p35 polypeptide, a peptide linker, and a hIL-12p40 polypeptide. In some embodiments, the schIL-12 polypeptide comprises from N- to C-terminus a hIL-12p40 polypeptide, a peptide linker, and a hIL-12p35 polypeptide.

In some embodiments, the hIL-12p40 polypeptide and the hIL-12p35 polypeptide arc operably connected via a peptide linker. In some embodiments, the peptide linker is of sufficient length such that the hIL-12p35 polypeptide and the hIL-12p40 polypeptide are able to associate such that the schIL-12 is able to bind the hIL-12 receptor. In some embodiments, the peptide linker comprises from about 5-30, 5-25, 5-20, 5-15, 10-30, 10-25, 10-20, or 10-15 amino acids. In some embodiments, the peptide linker comprises or consists of glycine (G) and serine(S) amino acid residues.

The amino acid sequence of exemplary linkers for use in schIL-12 polypeptides (to operably connect the hIL-12p35 polypeptide to the hIL-12p40 polypeptide of the schIL-12 polypeptide) are provided in Table 7.

TABLE 7
The Amino Acid Sequence of Exemplary Peptide
Linkers

| | | SEQ |
|---|---|---|
| | | ID |
| Description | Amino Acid Sequence | NO |
| Linker A | GGGS | 66 |
| Linker B | GGGSGGGS | 67 |
| Linker C | GGGSGGGSGGGS | 68 |
| Linker D | GGGSGGGSGGGSGGGS | 69 |
| Linker E | GGGGS | 70 |
| Linker F | GGGGSGGGGS | 71 |
| Linker G | GGGGSGGGGSGGGGS | 72 |
| Linker H | GGGGGGGGSGGGGSGGGGS | 73 |
| Linker I | GGGGGGGS | 74 |
| Linker J | GGGGGGGSGGGGGGGS | 75 |
| Linker K | GGGGGGGSGGGGGGGSGGGGGGGS | 76 |
| Linker L | GGGGGGGSGGGGGGGSGGGGGGGSGGGGGGGS | 77 |
| Linker M | SGGGG | 78 |
| Linker N | SGGGGSGGGG | 79 |
| Linker O | SGGGGSGGGGSGGGG | 80 |
| Linker P | SGGGGSGGGGSGGGGSGGGG | 81 |
| Linker EE | GGGGGGS | 369 |

In some embodiments, the amino acid sequence of the peptide linker comprises the amino acid sequence of any peptide linker set forth in Table 7; or the amino acid sequence of any peptide linker set forth in Table 7 comprising 1, 2, or 3 amino acid modifications (e.g., a substitution, deletion, or addition). In some embodiments, the peptide linker comprises the amino acid sequence of any one of SEQ ID NOs: 66-81 or 369, or the amino acid sequence of any one of SEQ ID NOS: 66-81 or 369 with 1, 2, or 3 amino acid modifications (e.g., a substitution, deletion, or addition). In some embodiments, the peptide linker comprises the amino acid sequence of any one of SEQ ID NO: 72, or the amino acid sequence of any one of SEQ ID NO: 72 with 1, 2, or 3 amino acid modifications (e.g., a substitution, deletion, or addition). In some embodiments, the peptide linker comprises the amino acid sequence of any one of SEQ ID NO: 369, or the amino acid sequence of any one of SEQ ID NO: 369 with 1, 2, or 3 amino acid modifications (e.g., a substitution, deletion, or addition).

### 5.2.6 Signal Peptides

In some embodiments, the hIL-12p40 and/or hIL-12p35 polypeptide comprises a homologous or heterologous signal peptide operably connected to the N-terminus of the hIL-12p40 and/or hIL-12p35 polypeptide.

In some embodiments, the hIL-12p40 polypeptide comprises an amino acid sequence set forth in any one of SEQ ID NOS: 38-65 and comprises a homologous signal peptide operably connected to the N-terminus of said polypeptide. In some embodiments, the hIL-12p40 polypeptide comprises an amino acid sequence set forth in any one of SEQ ID NOS: 38-65 and comprises a heterologous signal peptide operably connected to the N-terminus of said polypeptide. In some embodiments, the hIL-12p40 polypeptide comprises an amino acid sequence set forth in SEQ ID NO: 38 and comprises a homologous signal peptide operably connected to the N-terminus of said polypeptide. In some embodiments, the hIL-12p40 polypeptide comprises an amino acid sequence set forth in SEQ ID NO: 38 and comprises a heterologous signal peptide operably connected to the N-terminus of said polypeptide.

In some embodiments, the hIL-12p35 polypeptide comprises an amino acid sequence set forth in any one of SEQ ID NOS: 110-114 and comprises a homologous signal peptide operably connected to the N-terminus of said polypeptide. In some embodiments, the hIL-12p35 polypeptide comprises an amino acid sequence set forth in any one of SEQ ID NOS: 110-114 and comprises a heterologous signal peptide operably connected to the N-terminus of said polypeptide. In some embodiments, the hIL-12p35 polypeptide comprises an amino acid sequence set forth in SEQ ID NO: 110 and comprises a homologous signal peptide operably connected to the N-terminus of said polypeptide. In some embodiments, the hIL-12p35 polypeptide comprises an amino acid sequence set forth in SEQ ID NO: 110 and comprises a heterologous signal peptide operably connected to the N-terminus of said polypeptide.

Commonly used signal peptides are known in the art, for example, the native signal peptide of human interleukin 2 (hIL-2), human oncostatin M (hOSM), human chymotrypsinogen (hCTRB1), human trypsinogen 2 (hTRY2), and human insulin (hINS). A person of ordinary skill can determine the appropriate signal peptide using standard methodology known in the art. The amino acid sequence of exemplary signal peptides is provided in Table 8; along with the native signal sequence of hIL-12p40.

TABLE 8
The amino acid sequence of exemplary signal
peptides

| | | SEQ |
|---|---|---|
| Description | Amino Acid Sequence | ID NO |
| hIL-12p40 | MCHQQLVISWESLVELASPLVA | 82 |
| hIL-12p35 | MCPARSLLLVATLVLLDHLSLA | 394 |
| hIL-2 | MYRMQLLSCIALSLALVINS | 83 |
| hOSM | MGVLLTQRTLLSLVLALLFPSMASM | 84 |
| hCTRB1 | MASLWLLSCFSLVGAAFG | 85 |
| hTRY2 | MNLLLILTFVAAAVA | 86 |
| hINS | MALWMRLLPLLALLALWGPDPAAA | 87 |

In some embodiments, the amino acid sequence of the signal peptide comprises or consists of the amino acid sequence of any one of the signal peptides set forth in Table 8. In some embodiments, the amino acid sequence of the signal peptide comprises or consists of the amino acid sequence of any one of the signal peptides set forth in Table 8, and further comprises 1 or more but less than 15% (less than 12%, less than 10%, less than 8%), amino acid modifications (e.g., amino acid substitutions, deletions, or additions). In some embodiments, the amino acid sequence of the signal peptide comprises or consists of the amino acid sequence of any one of the signal peptides set forth in Table 8, comprising 1, 2, or 3 amino acid modifications (e.g., substitutions, deletions, additions).

In some embodiments, the amino acid sequence of the signal peptide comprises or consists of the amino acid sequence of any one of SEQ ID NOS: 82-87 or 394. In some embodiments, the amino acid sequence of the signal peptide comprises or consists of the amino acid sequence of any one of SEQ ID NOS: 82-87 or 394, and further comprises 1 or more but less than 15% (less than 12%, less than 10%, less than 8%), amino acid modifications (e.g., amino acid substitutions, deletions, or additions). In some embodiments, the amino acid sequence of the signal peptide comprises or consists of the amino acid sequence of any one of SEQ ID NOS: 82-87 or 394, comprising 1, 2, or 3 amino acid modifications (e.g., substitutions, deletions, additions).

### 5.3 hIL-12 Fusion Proteins and Conjugates

In one aspect, provided herein are fusion proteins comprising hIL-12 and a heterologous moiety (e.g., an antibody (e.g., a full-length antibody), an Fc region, etc.). In some embodiments, the fusion protein comprises an antibody and hIL-12 (see, e.g., § 5.3.3). In some embodiments, the fusion protein comprises an Fc region and a hIL-12 (see, e.g., § 5.3.2).

In some embodiments, the fusion protein comprises a half-life extension moiety. Exemplary half-life extension moieties include, but are not limited to, a human immunoglobulin (hIg), a fragment of a hIg, a hIg constant region, a fragment of a hIg constant region, an Fc region, human transferrin, human serum albumin (HSA), an HSA binding protein or peptide, and polyethylene glycol (PEG) (and polymers thereof). In some embodiments, the fusion protein comprises is a half-life extension polypeptide. Exemplary half-life extension polypeptides include, but are not limited to, a hIg, a fragment of a hIg, one or more hIg heavy chain constant region, a fragment of a hIg constant region, a hIg Fc region, human transferrin, human serum albumin (HSA), and an HSA binding protein or peptide. The hIL-12 polypeptide fused or conjugated to a half-life extending moiety or a half-life extending moiety can be evaluated for their pharmacokinetic properties utilizing standard in vitro methods known in the art.

5.3.1 hIL-12 Proteins & Polypeptides

As set forth above, the fusion proteins described herein comprise a hIL-12 protein. In some embodiments, the amino acid sequence of at least one subunit (e.g., hIL-12p40 or hIL-12p35) of the hIL-12 protein comprises or consists of the amino acid sequence of a naturally occurring subunit (e.g., hIL-12p40 or hIL-12p35). In some embodiments, the amino acid sequence of the hIL-12p35 subunit of the hIL-12 protein comprises or consists of the amino acid sequence of a naturally occurring hIL-12p35 protein (e.g., SEQ ID NO: 31). In some embodiments, the amino acid sequence of the IL-12p40 of the hIL-12 protein comprises or consists of the amino acid sequence of a naturally occurring hIL-12p40 protein (e.g., SEQ ID NO: 33).

In some embodiments, the amino acid sequence of the hIL-12p40 of the hIL-12 protein comprises at least one amino acid modification relative to the amino acid sequence of a reference hIL-12p40 protein (e.g., a naturally occurring hIL-12p40 protein, e.g., SEQ ID NO: 33). In some embodiments, the amino acid sequence of the hIL-12p35 subunit of the hIL-12 protein comprises at least one amino acid modification relative to the amino acid sequence of a reference hIL-12p35 protein (e.g., a naturally occurring hIL-12p35 protein, e.g., SEQ ID NO: 31). In some embodiments, the amino acid sequence of the hIL-12p40 subunit of the hIL-12 protein comprises at least one amino acid modification relative to the amino acid sequence of a reference hIL-12p40 protein (e.g., a naturally occurring hIL-12p40 protein, e.g., SEQ ID NO: 33); and the amino acid sequence of the hIL-12p35 subunit of the hIL-12 protein comprises at least one amino acid modification relative to the amino acid sequence of a reference hIL-12p35 protein (e.g., a naturally occurring hIL-12p35 protein, e.g., SEQ ID NO: 31).

In some embodiments, the amino acid sequence of the hIL-12p35 subunit of the hIL-12 protein comprises or consists of the amino acid sequence of a naturally occurring hIL-12p35 protein and the amino acid sequence of the hIL-12p40 subunit of the hIL-12 protein comprises at least one amino acid modification relative to the amino acid sequence of a reference hIL-12p40 protein (e.g., a naturally occurring hIL-12p40 protein, e.g., SEQ ID NO: 31). In some embodiments, the amino acid sequence of the hIL-12p40 subunit of the hIL-12 protein comprises or consists of the amino acid sequence of a naturally occurring hIL-12p40 protein and the amino acid sequence of the hIL-12p35 subunit of the hIL-12 protein comprises at least one amino acid modification relative to the amino acid sequence of a reference hIL-12p35 protein (e.g., a naturally occurring hIL-12p35, e.g., SEQ ID NO: 31).

5.3.1.1 hIL-12p40 Subunit

As set forth above, the fusion proteins and polypeptides described herein comprise a hIL-12 protein that comprises a hIL-12p40 subunit.

In some embodiments, the amino acid sequence of the IL-12p40 of the hIL-12 protein comprises or consists of the amino acid sequence of a naturally occurring hIL-12p40 protein (e.g., SEQ ID NO: 33). In some embodiments, the amino acid sequence of the IL-12p40 of the hIL-12 protein comprises or consists of the amino acid sequence of SEQ ID NO: 33. In some embodiments, the amino acid sequence of the hIL-12p40 subunit of the hIL-12 protein comprises at least one amino acid modification relative to the amino acid sequence of a reference hIL-12p40 protein (e.g., a naturally occurring hIL-12p40 protein, e.g., SEQ ID NO: 33).

In some embodiments, the hIL-12p40 subunit is a hIL-12p40 polypeptide described herein in see, e.g., § 5.2 (e.g., §§ 5.2.1, 5.2.2, 5.2.5, and 5.2.6). The full disclosure of § 5.2 (e.g., §§ 5.2.1, 5.2.2, 5.2.5, and 5.2.6), is incorporated in this instant § 5.3.1.1 by reference. Any of the hIL-12p40 polypeptides and embodiments provided in § 5.2 can be incorporated into a fusion protein described herein (e.g., an antibody (e.g., anti-CAIX antibody) fusion protein). In some embodiments, the hIL-12p40 subunit is a hIL-12p40 polypeptide described in § 5.2 (e.g., §§ 5.2.1, 5.2.2, 5.2.5, and 5.2.6).

In some embodiments, the hIL-12p40 polypeptide comprises or consists of an amino acid sequence (a) at least 85%, 86%, 87%, 88%, 89%, 90%, 91%, 92%, 93%, 94%, 95%, 96%, 97%, 98%, or 99%, identical to the amino acid sequence of SEQ ID NO: 33; and (b) comprises or consists of an amino acid substitution at each of amino acid positions (i) K280, K282, R283, K285, K286, and R288; (ii) E81, K121, and K286; (iii) S205, L206, P207, 1208, E209, and V210; (iv) S205, L206, P207, 1208, E209, and V210; (v) S205, L206, P207, 1208, E209, and V210; (vi) K217, L218, K219, Y220, and E221; (vii) E81 and F82; (viii) E81, F82, and K106; (xiv) E81, F82, K106, and K217; (xv) P39, D40, E81, and F82; (xvi) W37, F82, and K217; (xvii) W37, F82, and K219; or (xviii) K106, K217, and K219; (xxix) W37, F82, K106, and K219; (xxx) H216, K217, and K219; (xxxi) P207; W37 and F82; (xxxii) W37 and K217; (xxxiii) W37 and K219; (xxxiv) W37 and K106; (xxxv) F82 and K106; (xxxvi) F82 and K217; (xxxvii) F82 and K219; (xxxviii) K217 and K219; (xxxix) K106 and K217; (xl) K106 and K219; (xli) W37; (xlii) F82; (xliii) K106; (xliv) K217; or (xlv) K219; amino acid numbering relative to the amino acid sequence of SEQ ID NO: 32.

In some embodiments, the hIL-12p40 polypeptide comprises or consists of an amino acid sequence (a) at least 85%, 86%, 87%, 88%, 89%, 90%, 91%, 92%, 93%, 94%, 95%, 96%, 97%, 98%, or 99%, identical to the amino acid sequence of SEQ ID NO: 33; and (b) comprises or consists of the following amino acid substitutions: (i) K280A, K282A, R283A, K285A, K286A, and R288A; (ii) E81K, K121E, and K286E; (iii) S205I, L184E, P207S, I208I, E209K, and V210S; (iv) S205I, L206V, P207S, I208I, E209K, V210I; (v) S205I, L206Q, P207S, I208I, E209K, V210G; (vi) K217S, L218I, K219T, Y220S, E221A; (vii) E81A and F82A; (viii) E81A, F82A, K106A; (xiv) E81A, F82A, K106A, and K217A; (xv) P39A, D40A, E81A, and F82A; (xvi) W37A, F82A, and K217A; (xvii) W37A, F82A, and K219A; (xviii) K106A, K217A, and K219A; (xxix) W37A, F82A, K106A, and K219A; (xxx) H216A, K217A, and K219A; (xxxi) P207S; W37A and F82A; (xxxii) W37 and K217A; (xxxiii) W37A and K219A; (xxxiv) W37A and K106A; (xxxv) F82A and K106A; (xxxvi) F82A and K217A; (xxxvii) F82A and K219A; (xxxviii) K217A and K219A; (xxxix) K106A and K217A; (xl) K106A and K219A; (xli) W37A; (xlii) F82A; (xliii) K106A; (xliv) K217A; or (xlv) K219A; amino acid numbering relative to the amino acid sequence of SEQ ID NO: 32.

In some embodiments, the hIL-12p40 polypeptide comprises the amino acid sequence of SEQ ID NO: 33, and further comprises a deletion of amino acid residues 23-127, amino acid numbering relative to the amino acid sequence of SEQ ID NO: 32.

In some embodiments, the hIL-12p40 polypeptide comprises the amino acid sequence of SEQ ID NO: 33, and further comprises a deletion of amino acid residues 208-328 and an amino acid substitution at each of the following amino acid positions S205, L206, P207, amino acid numbering relative to the amino acid sequence of SEQ ID NO: 32. In some embodiments, the hIL-12p40 polypeptide comprises the amino acid sequence of SEQ ID NO: 33, and further comprises a deletion of amino acid residues 208-328 and each of the following amino acid substitutions S205I, L206E, P207S, amino acid numbering relative to the amino acid sequence of SEQ ID NO: 32.

In some embodiments, the hIL-12p40 polypeptide comprises the amino acid sequence of SEQ ID NO: 33, and further comprises the substitution of amino acids K217 and L218 with I, amino acid numbering relative to the amino acid sequence of SEQ ID NO: 32.

In some embodiments, the hIL-12p40 polypeptide comprises a modified heparin binding domain. In some embodiments, the hIL-12p40 polypeptide comprises a modified heparin binding which disrupts, inhibits, or reduces the ability of the hIL-12p40 polypeptide to bind a heparin compound as compared to a reference hIL-12p40 polypeptide that does not contain the modification in the heparin binding domain. In some embodiments, the hIL-12p40 polypeptide comprises a modified heparin binding domain and exhibits substantially the same, more, or less, immunostimulatory activity than that of a reference hIL-12p40 polypeptide that does not contain the modification in the heparin binding domain. In some embodiments, the hIL-12p40 polypeptide comprises a modified heparin binding domain and exhibits substantially the same immunostimulatory activity than that of a reference hIL-12p40 polypeptide that does not contain the modification in the heparin binding domain.

In some embodiments, the unmodified heparin binding domain of the hIL-12p40 polypeptide comprises or consist of the following amino acid sequence of SEQ ID NO: 88. In some embodiments, the modified heparin binding domain comprises or consists essentially of or consists of the amino acid sequence of SEQ ID NO: 89.

The amino acid sequence of a hIL-12p40 reference heparin binding domain and variants thereof is provided in Table 9.

TABLE 9
hIL-12p40 Heparin Binding Domain and Variants Thereof

| | | | SEQ ID |
|---|---|---|---|
| Description | Amino Acid Sequence | | NO |
| Reference Unmodified | VQVQGKSKREKK | | 88 |
| Heparin Binding Domain | | | |
| Modified Heparin | VX1X2QX3K*X4X5X6 | wherein | 89 |
| Binding Domain | X7K*X8 | X1 is R or Q, | |
| | | X2 is V, A, or I, | |
| | | X3 is G or R*, | |
| | | X4 is S, N, or K*, | |
| | | X5 is K*, N, or E, | |
| | | X6 is R or K, | |
| | | X7 is E, M, or T, and | |
| | | X8 is K* or E, and | |
| | | wherein one or more amino | |
| | | acid residues designated | |
| | | with an “*” are substituted | |
| | | with a non-polar amino | |
| | | acid residue selected from | |
| | | the group consisting of A, | |
| | | G, I, L, M, F, P, and V | |

In some embodiments, the one or more amino acid residues designated with an “*” in SEQ ID NO: 89 are alanine. In some embodiments, each of the amino acid residues designated with an “*” in SEQ ID NO: 89 are alanine. In some embodiments, amino acid residue X3 is alanine.

Exemplary hIL-12p40 polypeptides with modified heparin binding domains are described in U.S. Pat. No. 8,617,557, the full contents of which is incorporated herein by reference for all purposes.

The amino acid sequence of exemplary hIL-12p40 polypeptides that can be incorporated into the fusion proteins described herein is provided in Table 10.

TABLE 10
The Amino Acid Sequences of Exemplary hIL-12p40 Polypeptides

| | SEQ ID | |
|---|---|---|
| Description | NO | Amino Acid Sequence |
| hIL-12p40 (Mature- | 33 | See Table 4. |
| No Signal Peptide) | | |
| hIL-12p40 | 90 | IWELKKDVYVVELDWYPDAPGEMVVLTCDTPEEDGITWTLDQSS |
| Variant A | | EVLGSGKTLTIQVKEFGDAGQYTCHKGGEVLSHSLLLLHKKEDG |
| (K280A, K282A, | | IWSTDILKDQKEPKNKTFLRCEAKNYSGRFTCWWLTTISTDLTF |
| R283A, K285A, | | SVKSSRGSSDPQGVTCGAATLSAERVRGDNKEYEYSVECQEDSA |
| K286A, R288A) | | CPAAEESLPIEVMVDAVHKLKYENYTSSFFIRDIIKPDPPKNLQ |
| | | LKPLKNSRQVEVSWEYPDTWSTPHSYESLTFCVQVQGASAAEAA |
| | | DAVFTDKTSATVICRKNASISVRAQDRYYSSSWSEWASVPCS |
| hIL-12p40 | 91 | FLRCEAKNYSGRFTCWWLTTISTDLTFSVKSSRGSSDPQGVTCG |
| Variant B | | AATLSAERVRGDNKEYEYSVECQEDSACPAAEESLPIEVMVDAV |
| (Δ23-127) | | HKLKYENYTSSFFIRDIIKPDPPKNLQLKPLKNSRQVEVSWEYP |
| | | DTWSTPHSYFSLTFCVQVQGKSKREKKDRVFTDKTSATVICRKN |
| | | ASISVRAQDRYYSSSWSEWASVPCS |
| hIL-12p40 | 92 | IWELKKDVYVVELDWYPDAPGEMVVLTCDTPEEDGITWTLDQSS |
| Variant C | | EVLGSGKTLTIQVKEFGDAGQYTCHKGGEVLSHSLLLLHKKEDG |
| (Δ208-328; S205I, | | IWSTDILKDQKEPKNKTFLRCEAKNYSGRFTCWWLTTISTDLTF |
| L206E, P207S) | | SVKSSRGSSDPQGVTCGAATLSAERVRGDNKEYEYSVECQEDSA |
| | | CPAAEEIES |
| hIL-12p40 | 93 | IWELKKDVYVVELDWYPDAPGEMVVLTCDTPEEDGITWILDQSS |
| Variant D | | EVLGSGKTLTIQVKKFGDAGQYTCHKGGEVLSHSLLLLHKKEDG |
| (E81K, K121E, | | IWSTDILKDQEEPKNKTFLRCEAKNYSGRFTCWWLTTISTDLTF |
| K286E) | | SVKSSRGSSDPQGVTCGAATLSAERVRGDNKEYEYSVECQEDSA |
| | | CPAAEESLPIEVMVDAVHKLKYENYTSSFFIRDIIKPDPPKNLQ |
| | | LKPLKNSRQVEVSWEYPDTWSTPHSYFSLTFCVQVQGKSKREKE |
| | | DRVFTDKTSATVICRKNASISVRAQDRYYSSSWSEWASVPCS |
| hIL-12p40 | 94 | IWELKKDVYVVELDWYPDAPGEMVVLTCDTPEEDGITWTLDQSS |
| Variant E | | EVLGSGKTLTIQVKEFGDAGQYTCHKGGEVLSHSLLLLHKKEDG |
| (S205I, L206E, P207S, | | IWSTDILKDQKEPKNKTFLRCEAKNYSGRFTCWWLTTISTDLTF |
| I208I, E209K, V210S) | | SVKSSRGSSDPQGVTCGAATLSAERVRGDNKEYEYSVECQEDSA |
| | | CPAAEEIESIKSMVDAVHKLKYENYTSSFFIRDIIKPDPPKNLQ |
| | | LKPLKNSRQVEVSWEYPDTWSTPHSYFSLTFCVQVQGKSKREKK |
| | | DRVFTDKTSATVICRKNASISVRAQDRYYSSSWSEWASVPCS |
| hIL-12p40 | 95 | IWELKKDVYVVELDWYPDAPGEMVVLTCDTPEEDGITWTLDQSS |
| Variant F | | EVLGSGKTLTIQVKEFGDAGQYTCHKGGEVLSHSLLLLHKKEDG |
| (S205I, L206V, P207S, | | IWSTDILKDQKEPKNKTFLRCEAKNYSGRFTCWWLTTISTDLTF |
| I208I, E209K, V210I) | | SVKSSRGSSDPQGVTCGAATLSAERVRGDNKEYEYSVECQEDSA |
| | | CPAAEEIVSIKIMVDAVHKLKYENYTSSFFIRDIIKPDPPKNLQ |
| | | LKPLKNSRQVEVSWEYPDTWSTPHSYFSLTFCVQVQGKSKREKK |
| | | DRVFTDKTSATVICRKNASISVRAQDRYYSSSWSEWASVPCS |
| hIL-12p40 | 96 | IWELKKDVYVVELDWYPDAPGEMVVLTCDTPEEDGITWTLDQSS |
| Variant G | | EVLGSGKTLTIQVKEFGDAGQYTCHKGGEVLSHSLLLLHKKEDG |
| (S205I, L206Q, P207S, | | IWSTDILKDQKEPKNKTFLRCEAKNYSGRFTCWWLTTISTDLTF |
| I208I, E209K, V210G) | | SVKSSRGSSDPQGVTCGAATLSAERVRGDNKEYEYSVECQEDSA |
| | | CPAAEEIQSIKGMVDAVHKLKYENYTSSFFIRDIIKPDPPKNLQ |
| | | LKPLKNSRQVEVSWEYPDTWSTPHSYFSLTFCVQVQGKSKREKK |
| | | DRVFTDKTSATVICRKNASISVRAQDRYYSSSWSEWASVPCS |
| hIL-12p40 | 51 | See Table 5. |
| Variant H | | |
| (mature-no signal | | |
| peptide) | | |
| (K217 + L218 | | |
| substituted with I) | | |
| hIL-12p40 | 97 | IWELKKDVYVVELDWYPDAPGEMVVLTCDTPEEDGITWTLDQSS |
| Variant I | | EVLGSGKTLTIQVKEFGDAGQYTCHKGGEVLSHSLLLLHKKEDG |
| (K217S, L218I, | | IWSTDILKDQKEPKNKTFLRCEAKNYSGRFTCWWLTTISTDLTF |
| K219T, Y220S, | | SVKSSRGSSDPQGVTCGAATLSAERVRGDNKEYEYSVECQEDSA |
| E221A) | | CPAAEESLPIEVMVDAVHSITSANYTSSFFIRDIIKPDPPKNLQ |
| | | LKPLKNSRQVEVSWEYPDTWSTPHSYFSLTFCVQVQGKSKREKK |
| | | DRVFTDKTSATVICRKNASISVRAQDRYYSSSWSEWASVPCS |
| hIL-12p40 | 98 | IWELKKDVYVVELDWYPDAPGEMVVLTCDTPEEDGITWTLDQSS |
| Variant J | | EVLGSGKTLTIQVKAAGDAGQYTCHKGGEVLSHSLLLLHKKEDG |
| (E81A, F82A) | | IWSTDILKDQKEPKNKTFLRCEAKNYSGRFTCWWLTTISTDLTF |
| | | SVKSSRGSSDPQGVTCGAATLSAERVRGDNKEYEYSVECQEDSA |
| | | CPAAEESLPIEVMVDAVHKLKYENYTSSFFIRDIIKPDPPKNLQ |
| | | LKPLKNSRQVEVSWEYPDTWSTPHSYFSLTFCVQVQGKSKREKK |
| | | DRVFTDKTSATVICRKNASISVRAQDRYYSSSWSEWASVPCS |
| hIL-12p40 | 99 | IWELKKDVYVVELDWYPDAPGEMVVLTCDTPEEDGITWTLDQSS |
| Variant K | | EVLGSGKTLTIQVKAAGDAGQYTCHKGGEVLSHSLLLLHAKEDG |
| (E81A, F82A, K106A) | | IWSTDILKDQKEPKNKTFLRCEAKNYSGRFTCWWLTTISTDLTF |
| | | SVKSSRGSSDPQGVTCGAATLSAERVRGDNKEYEYSVECQEDSA |
| | | CPAAEESLPIEVMVDAVHKLKYENYTSSFFIRDIIKPDPPKNLQ |
| | | LKPLKNSRQVEVSWEYPDTWSTPHSYFSLTFCVQVQGKSKREKK |
| | | DRVFTDKTSATVICRKNASISVRAQDRYYSSSWSEWASVPCS |
| hIL-12p40 | 100 | IWELKKDVYVVELDWYPDAPGEMVVLTCDTPEEDGITWTLDQSS |
| Variant L | | EVLGSGKTLTIQVKAAGDAGQYTCHKGGEVLSHSLLLLHAKEDG |
| (E81A, F82A, K106A, | | IWSTDILKDQKEPKNKTFLRCEAKNYSGRFTCWWLTTISTDLTF |
| K217A) | | SVKSSRGSSDPQGVTCGAATLSAERVRGDNKEYEYSVECQEDSA |
| | | CPAAEESLPIEVMVDAVHALKYENYTSSFFIRDIIKPDPPKNLQ |
| | | LKPLKNSRQVEVSWEYPDTWSTPHSYFSLTFCVQVQGKSKREKK |
| | | DRVFTDKTSATVICRKNASISVRAQDRYYSSSWSEWASVPCS |
| hIL-12p40 | 101 | IWELKKDVYVVELDWYAAAPGEMVVLTCDTPEEDGITWTLDQSS |
| Variant M | | EVLGSGKTLTIQVKAAGDAGQYTCHKGGEVLSHSLLLLHKKEDG |
| (P39A, D40A, E81A, | | IWSTDILKDQKEPKNKTFLRCEAKNYSGRFTCWWLTTISTDLTF |
| F82A) | | SVKSSRGSSDPQGVTCGAATLSAERVRGDNKEYEYSVECQEDSA |
| | | CPAAEESLPIEVMVDAVHKLKYENYTSSFFIRDIIKPDPPKNLQ |
| | | LKPLKNSRQVEVSWEYPDTWSTPHSYFSLTFCVQVQGKSKREKK |
| | | DRVFTDKTSATVICRKNASISVRAQDRYYSSSWSEWASVPCS |
| hIL-12p40 | 39 | See Table 5. |
| Variant N | | |
| (W37A/F82A/K217A) | | |
| hIL-12p40 | 38 | See Table 5. |
| Variant O | | |
| (W37A/F82A/K219A) | | |
| hIL-12p40 | 40 | See Table 5. |
| Variant P | | |
| (K106A/K217A/K219A) | | |
| hIL-12p40 | 102 | IWELKKDVYVVELDAYPDAPGEMVVLTCDTPEEDGITWTLDQSS |
| Variant Q | | EVLGSGKTLTIQVKEAGDAGQYTCHKGGEVLSHSLLLLHAKEDG |
| (W37A, F82A, K106A, | | IWSTDILKDQKEPKNKTFLRCEAKNYSGRFTCWWLTTISTDLTF |
| K219A) | | SVKSSRGSSDPQGVTCGAATLSAERVRGDNKEYEYSVECQEDSA |
| | | CPAAEESLPIEVMVDAVHKLAYENYTSSFFIRDIIKPDPPKNLQ |
| | | LKPLKNSRQVEVSWEYPDTWSTPHSYFSLTFCVQVQGKSKREKK |
| | | DRVFTDKTSATVICRKNASISVRAQDRYYSSSWSEWASVPCS |
| hIL-12p40 | 103 | IWELKKDVYVVELDWYPDAPGEMVVLTCDTPEEDGITWTLDQSS |
| Variant R | | EVLGSGKTLTIQVKEFGDAGQYTCHKGGEVLSHSLLLLHKKEDG |
| (H216A, K217A, | | IWSTDILKDQKEPKNKTFLRCEAKNYSGRFTCWWLTTISTDLTF |
| K219A) | | SVKSSRGSSDPQGVTCGAATLSAERVRGDNKEYEYSVECQEDSA |
| | | CPAAEESLPIEVMVDAVAALAYENYTSSFFIRDIIKPDPPKNLQ |
| | | LKPLKNSRQVEVSWEYPDTWSTPHSYFSLTFCVQVQGKSKREKK |
| | | DRVFTDKTSATVICRKNASISVRAQDRYYSSSWSEWASVPCS |
| hIL-12p40 | 104 | IWELKKDVYVVELDWYPDAPGEMVVLTCDTPEEDGITWTLDQSS |
| Variant S | | EVLGSGKTLTIQVKEFGDAGQYTCHKGGEVLSHSLLLLHKKEDG |
| (P207S) | | IWSTDILKDQKEPKNKTFLRCEAKNYSGRFTCWWLTTISTDLTF |
| | | SVKSSRGSSDPQGVTCGAATLSAERVRGDNKEYEYSVECQEDSA |
| | | CPAAEESLSIEVMVDAVHKLKYENYTSSFFIRDIIKPDPPKNLQ |
| | | LKPLKNSRQVEVSWEYPDTWSTPHSYFSLTFCVQVQGKSKREKK |
| | | DRVFTDKTSATVICRKNASISVRAQDRYYSSSWSEWASVPCS |
| hIL-12p40 | 41 | See Table 5. |
| Variant T | | |
| (W37A/F82A) | | |
| hIL-12p40 | 42 | See Table 5. |
| Variant U | | |
| (W37A/K217A) | | |
| hIL-12p40 | 43 | See Table 5. |
| Variant V | | |
| (W37A/K219A) | | |
| hIL-12p40 | 44 | See Table 5. |
| Variant W | | |
| (W37A/K106A) | | |
| hIL-12p40 | 45 | See Table 5. |
| Variant X | | |
| (F82A/K106A) | | |
| hIL-12p40 | 46 | See Table 5. |
| Variant Y | | |
| (F82A/K217A) | | |
| hIL-12p40 | 47 | See Table 5. |
| Variant Z | | |
| (F82A/K219A) | | |
| hIL-12p40 | 48 | See Table 5. |
| Variant AA | | |
| (K217A/K219A) | | |
| hIL-12p40 | 49 | See Table 5. |
| Variant BB | | |
| (K106A/K217A) | | |
| hIL-12p40 | 50 | See Table 5. |
| Variant CC | | |
| (K106A/K219A) | | |
| hIL-12p40 | 105 | IWELKKDVYVVELDAYPDAPGEMVVLTCDTPEEDGITWTLDQSS |
| Variant DD | | EVLGSGKTLTIQVKEFGDAGQYTCHKGGEVLSHSLLLLHKKEDG |
| (W37A) | | IWSTDILKDQKEPKNKTFLRCEAKNYSGRFTCWWLTTISTDLTF |
| | | SVKSSRGSSDPQGVTCGAATLSAERVRGDNKEYEYSVECQEDSA |
| | | CPAAEESLPIEVMVDAVHKLKYENYTSSFFIRDIIKPDPPKNLQ |
| | | LKPLKNSRQVEVSWEYPDTWSTPHSYFSLTFCVQVQGKSKREKK |
| | | DRVFTDKTSATVICRKNASISVRAQDRYYSSSWSEWASVPCS |
| hIL-12p40 | 106 | IWELKKDVYVVELDWYPDAPGEMVVLTCDTPEEDGITWTLDQSS |
| Variant EE | | EVLGSGKTLTIQVKEAGDAGQYTCHKGGEVLSHSLLLLHKKEDG |
| (F82A) | | IWSTDILKDQKEPKNKTFLRCEAKNYSGRFTCWWLTTISTDLTF |
| | | SVKSSRGSSDPQGVTCGAATLSAERVRGDNKEYEYSVECQEDSA |
| | | CPAAEESLPIEVMVDAVHKLKYENYTSSFFIRDIIKPDPPKNLQ |
| | | LKPLKNSRQVEVSWEYPDTWSTPHSYFSLTFCVQVQGKSKREKK |
| | | DRVFTDKTSATVICRKNASISVRAQDRYYSSSWSEWASVPCS |
| hIL-12p40 | 107 | IWELKKDVYVVELDWYPDAPGEMVVLTCDTPEEDGITWTLDQSS |
| Variant FF | | EVLGSGKTLTIQVKEFGDAGQYTCHKGGEVLSHSLLLLHAKEDG |
| (K106A) | | IWSTDILKDQKEPKNKTFLRCEAKNYSGRFTCWWLTTISTDLTF |
| | | SVKSSRGSSDPQGVTCGAATLSAERVRGDNKEYEYSVECQEDSA |
| | | CPAAEESLPIEVMVDAVHKLKYENYTSSFFIRDIIKPDPPKNLQ |
| | | LKPLKNSRQVEVSWEYPDTWSTPHSYFSLTFCVQVQGKSKREKK |
| | | DRVFTDKTSATVICRKNASISVRAQDRYYSSSWSEWASVPCS |
| hIL-12p40 | 108 | IWELKKDVYVVELDWYPDAPGEMVVLTCDTPEEDGITWTLDQSS |
| Variant GG | | EVLGSGKTLTIQVKEFGDAGQYTCHKGGEVLSHSLLLLHKKEDG |
| (K217A) | | IWSTDILKDQKEPKNKTFLRCEAKNYSGRFTCWWLTTISTDLTF |
| | | SVKSSRGSSDPQGVTCGAATLSAERVRGDNKEYEYSVECQEDSA |
| | | CPAAEESLPIEVMVDAVHALKYENYTSSFFIRDIIKPDPPKNLQ |
| | | LKPLKNSRQVEVSWEYPDTWSTPHSYFSLTFCVQVQGKSKREKK |
| | | DRVFTDKTSATVICRKNASISVRAQDRYYSSSWSEWASVPCS |
| hIL-12p40 | 109 | IWELKKDVYVVELDWYPDAPGEMVVLTCDTPEEDGITWTLDOSS |
| Variant HH | | EVLGSGKTLTIQVKEFGDAGQYTCHKGGEVLSHSLLLLHKKEDG |
| (K219A) | | IWSTDILKDQKEPKNKTFLRCEAKNYSGRFTCWWLTTISTDLTF |
| | | SVKSSRGSSDPQGVTCGAATLSAERVRGDNKEYEYSVECQEDSA |
| | | CPAAEESLPIEVMVDAVHKLAYENYTSSFFIRDIIKPDPPKNLQ |
| | | LKPLKNSRQVEVSWEYPDTWSTPHSYFSLTFCVQVQGKSKREKK |
| | | DRVFTDKTSATVICRKNASISVRAQDRYYSSSWSEWASVPCS |

In some embodiments, the amino acid sequence of the hIL-12p40 polypeptide comprises an amino acid sequence at least 85%, 86%, 87%, 88%, 89%, 90%, 91%, 92%, 93%, 94%, 95%, 96%, 97%, 98%, 99%, or 100% identical to the amino acid sequence of any one of the polypeptides set forth in Table 10.

In some embodiments, the amino acid sequence of the hIL-12p40 polypeptide comprises an amino acid sequence at least 85%, 86%, 87%, 88%, 89%, 90%, 91%, 92%, 93%, 94%, 95%, 96%, 97%, 98%, 99%, or 100% identical to the amino acid sequence of any one of SEQ ID NOS: 38-51 or 90-109.

In some embodiments, the amino acid sequence of the hIL-12p35 polypeptide comprises or consists of a set of amino acid modifications (e.g., substitutions, deletions) set forth in the amino acid sequence of any one polypeptide set forth in Table 10 (relative to the amino acid sequence of SEQ ID NO: 33); and other than the set of amino acid modifications (e.g., substitutions, deletions), the amino acid sequence of the hIL-12p40 polypeptide is at least 85%, 86%, 87%, 88%, 89%, 90%, 91%, 92%, 93%, 94%, 95%, 96%, 97%, 98%, 99%, or 100% identical to the amino acid sequence of the polypeptide set forth in Table 10.

In some embodiments, the amino acid sequence of the hIL-12p40 polypeptide comprises or consists of a set of amino acid modifications (e.g., substitutions, deletions) set forth in the amino acid sequence set forth in any one of SEQ ID NOS: 38-51 or 90-109 (relative to the amino acid sequence of SEQ ID NO: 33); and other than the set of amino acid modifications (e.g., substitutions, deletions), the amino acid sequence of the hIL-12p40 polypeptide is at least 85%, 86%, 87%, 88%, 89%, 90%, 91%, 92%, 93%, 94%, 95%, 96%, 97%, 98%, 99%, or 100% identical to the amino acid sequence set forth in the any one of SEQ ID NOS: 38-51 or 90-109.

In some embodiments, the amino acid sequence of the hIL-12p40 polypeptide comprises or consists of a set of amino acid modifications (e.g., substitutions, deletions) set forth in the amino acid sequence set forth in SEQ ID NO: 38 (relative to the amino acid sequence of SEQ ID NO: 33); and other than the set of amino acid modifications (e.g., substitutions, deletions), the amino acid sequence of the hIL-12p40 polypeptide is at least 85%, 86%, 87%, 88%, 89%, 90%, 91%, 92%, 93%, 94%, 95%, 96%, 97%, 98%, 99%, or 100% identical to the amino acid sequence set forth in the SEQ ID NO: 38.

5.3.1.2 hIL-12p35 Subunit

As set forth above, the fusion proteins and polypeptides described herein comprise a hIL-12 protein that comprises a hIL-12p35 subunit.

In some embodiments, the amino acid sequence of the hIL-12p35 subunit of the hIL-12 protein comprises or consists of the amino acid sequence of a naturally occurring hIL-12p35 protein (e.g., SEQ ID NO: 31). In some embodiments, the amino acid sequence of the hIL-12p35 subunit of the hIL-12 protein comprises at least one amino acid modification relative to the amino acid sequence of a reference hIL-12p35 protein (e.g., a naturally occurring hIL-12p35 protein, e.g., SEQ ID NO: 31).

In some embodiments, the hIL-12p35 subunit is a hIL-12p35 polypeptide described herein in see, e.g., § 5.2 (e.g., §§ 5.2.3, 5.2.4, 5.2.5, and 5.2.6). The full disclosure of § 5.2 (e.g., §§ 5.2.3, 5.2.4, 5.2.5, and 5.2.6), is incorporated in this instant § 5.3.1.2 by reference. Any of the hIL-12p35 polypeptides and embodiments provided in § 5.2 (e.g., §§ 5.2.3, 5.2.4, 5.2.5, and 5.2.6) can be incorporated into a fusion protein described herein (e.g., an antibody (e.g., anti-CAIX antibody) fusion protein). In some embodiments, the hIL-12p35 subunit is a hIL-12p35 polypeptide described in § 5.2 (e.g., §§ 5.2.3, 5.2.4, 5.2.5, and 5.2.6).

In some embodiments, the at least one amino acid modification reduces binding affinity of the hIL-12p35 subunit for the hIL-12R. In some embodiments, the at least one amino acid modification reduces binding affinity of the hIL-12p35 subunit for the hIL-12Rβ1. In some embodiments, the at least one amino acid modification reduces binding affinity of the hIL-12p35 subunit for the hIL-12Rβ2. In some embodiments, the at least one amino acid modification reduces binding affinity of the hIL-12p35 subunit for the hIL-12Rβ1 and hIL-12Rβ1.

As set for the above, for the purposes of the instant disclosure, the numbering of all amino acids (and e.g., amino acid substitutions) of hIL-12p35 polypeptides described herein is set out relative to the amino acid sequence of the immature form of hIL-12p35 (i.e., SEQ ID NO: 30), that contains the native signal peptide. The use of the immature form of hIL-12p35 to designate amino acid numbers (e.g., Y189) is for consistency only and does not limit the scope of embodiments utilizing this numbering scheme to polypeptides that contain the signal peptide of hIL-12p35. For example, a hIL-12p35 polypeptide described herein as comprising the amino acid sequence of SEQ ID NO: 31 with a Y189A amino acid substitution does not require the signal peptide of hIL-12p35, although the numbering of amino acid position Y189 is based on the immature form of the protein. It common in the art to utilize the mature form of a protein to produce variants and fusion proteins.

A person of ordinary skill in art can easily determine the amino acid position in the mature form of hIL-12p35 (SEQ ID NO: 31) based on the amino acid numbering relative to the immature form of hIL-12p35. As set forth above, amino acids 1-22 of the immature form of the hIL-12p35 protein are the signal sequence. Therefore, an amino acid position of a particular amino acid in the mature form of the hIL-12p35 protein can be determined from the amino acid position of the particular amino acid designated relative to the immature form of hIL-12p35 by subtracting 22. For example, the amino acid position Y189 (numbering relative to SEQ ID NO: 30) would correspond to amino acid position Y167 in the mature form of the protein (SEQ ID NO: 31).

In some embodiments, the amino acid sequence of the hIL-12p35 polypeptide comprises or consists of an amino acid modification (e.g., substitution, addition, or deletion) at one or more of amino acid positions E60, F61, P63, K150, F188, or Y189, amino acid numbering relative to the amino acid sequence set forth SEQ ID NO: 30. In some embodiments, the amino acid sequence of the hIL-12p35 polypeptide comprises or consists of an amino acid modification (e.g., substitution, addition, or deletion) at 1, 2, 3, 4, 5, 6, or 7 of the following amino acid positions E60, F61, P63, K150, F188, or Y189, amino acid numbering relative to the amino acid sequence set forth SEQ ID NO: 30.

In some embodiments, the amino acid sequence of the hIL-12p35 polypeptide comprises or consists of an amino acid modification (e.g., substitution, addition, or deletion) at one or more of amino acid positions E60, F61, P63, K150, and F188, amino acid numbering relative to the amino acid sequence set forth SEQ ID NO: 30. In some embodiments, the amino acid sequence of the hIL-12p35 polypeptide comprises or consists of an amino acid modification (e.g., substitution, addition, or deletion) at one or more of amino acid positions F188 and Y189, amino acid numbering relative to the amino acid sequence set forth SEQ ID NO: 30. In some embodiments, the amino acid sequence of the hIL-12p35 polypeptide comprises or consists of an amino acid modification (e.g., substitution, addition, or deletion) at amino acid position F188, amino acid numbering relative to the amino acid sequence set forth SEQ ID NO: 30. In some embodiments, the amino acid sequence of the hIL-12p35 polypeptide comprises or consists of an amino acid modification (e.g., substitution, addition, or deletion) at amino acid position Y189A, amino acid numbering relative to the amino acid sequence set forth SEQ ID NO: 30.

In some embodiments, the amino acid sequence of the hIL-12p35 polypeptide comprises or consists of one or more of the following amino acid substitutions: E60K, F61H, P63S, K150H, F188P, F188A, or Y189A amino acid numbering relative to the amino acid sequence set forth SEQ ID NO: 30. In some embodiments, the amino acid sequence of the hIL-12p35 polypeptide comprises or consists of the following amino acid substitutions: E60K, F61H, P63S, K150H, and F188P, amino acid numbering relative to the amino acid sequence set forth SEQ ID NO: 30. In some embodiments, the amino acid sequence of the hIL-12p35 polypeptide comprises or consists of the following amino acid substitutions: F188A and Y189A, amino acid numbering relative to the amino acid sequence set forth SEQ ID NO: 30. In some embodiments, the amino acid sequence of the hIL-12p35 polypeptide comprises or consists of the following amino acid substitution F188A, amino acid numbering relative to the amino acid sequence set forth SEQ ID NO: 30. In some embodiments, the amino acid sequence of the hIL-12p35 polypeptide comprises or consists of the following amino acid substitution Y189A, amino acid numbering relative to the amino acid sequence set forth SEQ ID NO: 30.

In some embodiments, the amino acid sequence of the hIL12-p35 polypeptide comprises a deletion of amino acid residues 50-95, 50-94, 50-93, 50-92, 50-91, 50-90, 50-89, 51-95, 51-94, 51-93, 51-92, 51-91, 51-90, 51-89, 52-95, 52-94, 52-93, 52-92, 52-91, 52-90, 52-89, 53-95, 53-94, 53-93, 53-92, 53-91, 53-90, 53-89, 54-95, 54-94, 54-93, 54-92, 54-91, 54-90, 54-89, 55-95, 55-94, 55-93, 55-92, 55-91, 55-90, or 55-89; amino acid numbering relative to the amino acid sequence set forth SEQ ID NO: 30. In some embodiments, the amino acid sequence of the hIL12-p35 polypeptide comprises a deletion of amino acid residues A55-K92; amino acid numbering relative to the amino acid sequence set forth SEQ ID NO: 30.

In some embodiments, the amino acid sequence of the hIL-12p35 polypeptide comprises an amino acid sequence at least 85%, 86%, 87%, 88%, 89%, 90%, 91%, 92%, 93%, 94%, 95%, 96%, 97%, 98%, 99%, or 100% identical to the amino acid sequence of any one of the polypeptides set forth in Table 6.

In some embodiments, the amino acid sequence of the hIL-12p35 polypeptide comprises an amino acid sequence at least 85%, 86%, 87%, 88%, 89%, 90%, 91%, 92%, 93%, 94%, 95%, 96%, 97%, 98%, 99%, or 100% identical to the amino acid sequence of any one of SEQ ID NOS: 31 or 110-114.

In some embodiments, the amino acid sequence of the hIL-12p35 polypeptide comprises or consists of a set of amino acid modifications (e.g., substitutions, deletions) set forth in the amino acid sequence of any one polypeptide set forth in Table 6 (relative to the amino acid sequence of SEQ ID NO: 31); and other than the set of amino acid modifications (e.g., substitutions, deletions), the amino acid sequence of the hIL-12p35 polypeptide is at least 85%, 86%, 87%, 88%, 89%, 90%, 91%, 92%, 93%, 94%, 95%, 96%, 97%, 98%, 99%, or 100% identical to the amino acid sequence of the polypeptide set forth in Table 6.

In some embodiments, the amino acid sequence of the hIL-12p35 polypeptide comprises or consists of a set of amino acid modifications (e.g., substitutions, deletions) set forth in the amino acid sequence of any one SEQ ID NOS: 110-114 (relative to the amino acid sequence of SEQ ID NO: 31); and other than the set of amino acid modifications (e.g., substitutions, deletions), the amino acid sequence of the hIL-12p35 polypeptide is at least 85%, 86%, 87%, 88%, 89%, 90%, 91%, 92%, 93%, 94%, 95%, 96%, 97%, 98%, 99%, or 100% identical to the amino acid sequence of set forth in the any one of SEQ ID NOS: 110-114.

5.3.1.3 schIL-12 Polypeptides

In some embodiments, the hIL-12 of a fusion protein described herein is in the form of a single polypeptide chain (referred to herein as schIL-12). The schIL-12 polypeptide can comprise any hIL-12p40 polypeptide described herein (see, e.g., § 5.2 (e.g., § 5.2.1) and § 5.3.1.1); and any hIL-12p35 polypeptide described herein (see, e.g., § 5.2 (e.g., § 5.2.3) and § 5.3.1.2). The schIL-12 polypeptide can comprise a schIL-12 polypeptide described herein in § 5.2.5.

In some embodiments, the amino acid sequence of the hIL-12p40 polypeptide is at least 85%, 86%, 87%, 88%, 89%, 90%, 91%, 92%, 93%, 94%, 95%, 96%, 97%, 98%, 99%, or 100% identical to the amino acid sequence of set forth in the any one of SEQ ID NOS: 38-51 or 90-109. In some embodiments, the amino acid sequence of the hIL-12p35 polypeptide is at least 85%, 86%, 87%, 88%, 89%, 90%, 91%, 92%, 93%, 94%, 95%, 96%, 97%, 98%, 99%, or 100% identical to the amino acid sequence of set forth in the any one of SEQ ID NOS: 31 or 110-114.

In some embodiments, the schIL-12 comprises a hIL-12p35 polypeptide directly fused to a hIL-12p40 polypeptide. In some embodiments, the schIL-12 polypeptide comprises from N- to C-terminus a hIL-12p35 polypeptide, an optional peptide linker, and a hIL-12p40 polypeptide. In some embodiments, the schIL-12 polypeptide comprises from N- to C-terminus a hIL-12p40 polypeptide, an optional peptide linker, and a hIL-12p35 polypeptide.

In some embodiments, the schIL-12 polypeptide comprises a hIL-12p35 polypeptide directly operably connected via a peptide bond. In some embodiments, the schIL-12 polypeptide comprises a hIL-12p35 polypeptide indirectly fused to a hIL-12p40 polypeptide via a peptide linker. In some embodiments, the schIL-12 polypeptide comprises from N- to C-terminus a hIL-12p35 polypeptide, a peptide linker, and a hIL-12p40 polypeptide. In some embodiments, the schIL-12 polypeptide comprises from N- to C-terminus a hIL-12p40 polypeptide, a peptide linker, and a hIL-12p35 polypeptide.

The amino acid sequence of exemplary linkers for use in schIL-12 polypeptides (to operably connect an hIL-12p35 polypeptide to an hIL-12p40 polypeptide are provided in Table 11.

In some embodiments, the hIL-12p40 polypeptide and the hIL-12p35 polypeptide are operably connected via a peptide linker. In some embodiments, the peptide linker is of sufficient length such that the hIL-12p35 polypeptide and the hIL-12p40 polypeptide are able to associate such that the schIL-12 is able to bind the hIL-12 receptor. In some embodiments, the peptide linker comprises from about 5-30, 5-25, 5-20, 5-15, 10-30, 10-25, 10-20, or 10-15 amino acids. In some embodiments, the peptide linker comprises or consists of glycine (G) and serine(S) amino acid residues.

In some embodiments, the peptide linker comprises the amino acid sequence of any one of SEQ ID NOs: 66-81, or the amino acid sequence of any one of SEQ ID NOS: 66-81, with 1, 2, or 3 amino acid modifications (e.g., a substitution, deletion, or addition). In some embodiments, the peptide linker comprises the amino acid sequence of any one of SEQ ID NO: 72, or the amino acid sequence of any one of SEQ ID NO: 72, with 1, 2, or 3 amino acid modifications (e.g., a substitution, deletion, or addition).

The amino acid sequence of exemplary schIL-12 polypeptides is provided in Table 11.

TABLE 11
The Amino Acid Sequence of exemplary human scIL-12 polypeptides

| | SEQ ID | |
|---|---|---|
| Description | NO | Amino Acid Sequence |
| Wild Type hIL- | 115 | RNLPVATPDPGMFPCLHHSQNLLRAVSNMLQKARQTLEFYPCTSEE |
| 12p35-linker-Wild | | IDHEDITKDKTSTVEACLPLELTKNESCLNSRETSFITNGSCLASR |
| Type hIL-12p40 | | KTSFMMALCLSSIYEDLKMYQVEFKTMNAKLLMDPKRQIFLDQNML |
| | | AVIDELMQALNENSETVPQKSSLEEPDFYKTKIKLCILLHAFRIRA |
| | | VTIDRVMSYLNASGGGGSGGGGSGGGGSIWELKKDVYVVELDWYPD |
| | | APGEMVVLTCDTPEEDGITWTLDQSSEVLGSGKTLTIQVKEFGDAG |
| | | QYTCHKGGEVLSHSLLLLHKKEDGIWSTDILKDQKEPKNKTFLRCE |
| | | AKNYSGRFTCWWLTTISTDLTFSVKSSRGSSDPQGVTCGAATLSAE |
| | | RVRGDNKEYEYSVECQEDSACPAAEESLPIEVMVDAVHKLKYENYT |
| | | SSFFIRDIIKPDPPKNLQLKPLKNSRQVEVSWEYPDTWSTPHSYES |
| | | LTFCVQVQGKSKREKKDRVFTDKTSATVICRKNASISVRAQDRYYS |
| | | SSWSEWASVPCS |
| Wild Type hIL- | 116 | IWELKKDVYVVELDWYPDAPGEMVVLTCDTPEEDGITWTLDQSSEV |
| 12p40-linker-Wild | | LGSGKTLTIQVKEFGDAGQYTCHKGGEVLSHSLLLLHKKEDGIWST |
| Type hIL-12p35 | | DILKDQKEPKNKTFLRCEAKNYSGRFTCWWLTTISTDLTFSVKSSR |
| | | GSSDPQGVTCGAATLSAERVRGDNKEYEYSVECQEDSACPAAEESL |
| | | PIEVMVDAVHKLKYENYTSSFFIRDIIKPDPPKNLQLKPLKNSRQV |
| | | EVSWEYPDTWSTPHSYFSLTFCVQVQGKSKREKKDRVFTDKTSATV |
| | | ICRKNASISVRAQDRYYSSSWSEWASVPCSGGGGSGGGGSGGGGSR |
| | | NLPVATPDPGMFPCLHHSQNLLRAVSNMLQKARQTLEFYPCTSEEI |
| | | DHEDITKDKTSTVEACLPLELTKNESCLNSRETSFITNGSCLASRK |
| | | TSFMMALCLSSIYEDLKMYQVEFKTMNAKLLMDPKRQIFLDQNMLA |
| | | VIDELMQALNENSETVPQKSSLEEPDFYKTKIKLCILLHAFRIRAV |
| | | TIDRVMSYLNAS |
| Wild Type hIL- | 117 | RNLPVATPDPGMFPCLHHSQNLLRAVSNMLQKARQTLEFYPCTSEE |
| 12p35-linker-hIL- | | IDHEDITKDKTSTVEACLPLELTKNESCLNSRETSFITNGSCLASR |
| 12p40 Variant O | | KTSFMMALCLSSIYEDLKMYQVEFKTMNAKLLMDPKRQIFLDQNML |
| | | AVIDELMQALNENSETVPQKSSLEEPDFYKTKIKLCILLHAFRIRA |
| | | VTIDRVMSYLNASGGGGSGGGGSGGGGSIWELKKDVYVVELDAYPD |
| | | APGEMVVLTCDTPEEDGITWTLDQSSEVLGSGKTLTIQVKEAGDAG |
| | | QYTCHKGGEVLSHSLLLLHKKEDGIWSTDILKDQKEPKNKTFLRCE |
| | | AKNYSGRFTCWWLTTISTDLTFSVKSSRGSSDPQGVTCGAATLSAE |
| | | RVRGDNKEYEYSVECQEDSACPAAEESLPIEVMVDAVHKLAYENYT |
| | | SSFFIRDIIKPDPPKNLQLKPLKNSRQVEVSWEYPDTWSTPHSYES |
| | | LTFCVQVQGKSKREKKDRVFTDKTSATVICRKNASISVRAQDRYYS |
| | | SSWSEWASVPCS |
| hIL-12p40 Variant | 118 | IWELKKDVYVVELDAYPDAPGEMVVLTCDTPEEDGITWTLDQSSEV |
| O-linker-Wild Type | | LGSGKTLTIQVKEAGDAGQYTCHKGGEVLSHSLLLLHKKEDGIWST |
| hIL-12p35 | | DILKDQKEPKNKTFLRCEAKNYSGRFTCWWLTTISTDLTFSVKSSR |
| | | GSSDPQGVTCGAATLSAERVRGDNKEYEYSVECQEDSACPAAEESL |
| | | PIEVMVDAVHKLAYENYTSSFFIRDIIKPDPPKNLQLKPLKNSRQV |
| | | EVSWEYPDTWSTPHSYFSLTFCVQVQGKSKREKKDRVFTDKTSATV |
| | | ICRKNASISVRAQDRYYSSSWSEWASVPCSGGGGSGGGGSGGGGS |
| | | RNLPVATPDPGMFPCLHHSQNLLRAVSNMLQKARQTLEFYPCTSEE |
| | | IDHEDITKDKTSTVEACLPLELTKNESCLNSRETSFITNGSCLASR |
| | | KTSFMMALCLSSIYEDLKMYQVEFKTMNAKLLMDPKRQIFLDQNML |
| | | AVIDELMQALNENSETVPQKSSLEEPDFYKTKIKLCILLHAFRIRA |
| | | VTIDRVMSYLNAS |

In some embodiments, the amino acid sequence of the schIL-12 polypeptide is at least 85%, 86%, 87%, 88%, 89%, 90%, 91%, 92%, 93%, 94%, 95%, 96%, 97%, 98%, 99%, or 100% identical to the amino acid sequence of any one of the polypeptides set forth in Table 11.

In some embodiments, the amino acid sequence of the schIL-12 polypeptide is at least 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 115-118.

5.3.1.4 Potency & Affinity of hIL-12 Fusion Proteins & Polypeptides

In some embodiments, the hIL-12 fusion protein mediates a lower increase in the level of STAT4 in cells expressing the hIL-12R on the surface relative to the increase in STAT4 mediated by a suitable control (e.g., a reference hIL-12 fusion protein (e.g., SEQ ID NOS: 371, 372, 383)). In some embodiments, the hIL-12 fusion protein mediates a lower increase in the level of phosphorylated STAT4 (pSTAT4) in cells expressing the hIL-12R on the surface relative to the increase in pSTAT4 mediated by a suitable control (e.g., a reference hIL-12 fusion protein (e.g., SEQ ID NOS: 371, 372, 383)). In some embodiments, the hIL-12 fusion protein mediates about a 0.5-fold, 1-fold, 2-fold, 5-fold, 10-fold, 100-fold, or 1000-fold lower increase in the level of phosphorylated STAT4 (pSTAT4) in cells expressing the hIL-12R on the surface relative to the increase in pSTAT4 mediated by a suitable control (e.g., a reference hIL-12 fusion protein (e.g., SEQ ID NOS: 371, 372, 383)). In some embodiments, the hIL-12 fusion protein mediates at least about a 0.5-fold, 1-fold, 2-fold, 5-fold, 10-fold, 100-fold, or 1000-fold lower increase in the level of phosphorylated STAT4 (pSTAT4) in cells expressing the hIL-12R on the surface relative to the increase in pSTAT4 mediated by a suitable control (e.g., a reference hIL-12 fusion protein (e.g., SEQ ID NOS: 371, 372, 383)). In some embodiments, the hIL-12 fusion protein mediates from about a 0.5-1000 fold, 0.5-100 fold, 0.5-10 fold, 0.5-5 fold, 0.5-2 fold, 1-1000 fold, 1-100 fold, 1-10 fold, 1-5 fold, 1-2 fold, 10-1000 fold, or 100-1000 fold lower increase in the level of phosphorylated STAT4 (pSTAT4) in cells expressing the hIL-12R on the surface relative to the increase in pSTAT4 mediated by a suitable control (e.g., a reference hIL-12 fusion protein (e.g., SEQ ID NOS: 371, 372, 383)). Assays suitable to measure the EC50 the hIL-12 fusion protein described herein are standard and known to the person of ordinary skill in the art, as described, inter alia, in § 5.2.3.

In some embodiments, the hIL-12 fusion protein mediates a lower increase the level of interferon gamma (IFN-γ) produced by expressing the hIL-12R on the surface relative to the increase in the level of IFN-γ produced in the presence of a suitable control (e.g., a reference hIL-12 fusion protein (e.g., SEQ ID NOS: 371, 372, 383)). In some embodiments, the hIL-12 fusion protein mediates a 0.5-fold, 1-fold, 2-fold, 5-fold, 10-fold, 100-fold, or 1000-fold lower increase in the level of IFN-γ produced by expressing the hIL-12R on the surface, relative to the increase in the level of IFN-γ produced in the presence of a suitable control (e.g., a reference hIL-12 fusion protein (e.g., SEQ ID NOS: 371, 372, 383)). In some embodiments, the hIL-12 fusion protein mediates at least about 0.5-fold, 1-fold, 2-fold, 5-fold, 10-fold, 100-fold, or 1000-fold lower increase in the level of IFN-γ produced by expressing the hIL-12R on the surface relative to the increase in the level of IFN-γ produced in the presence of a suitable control (e.g., a reference hIL-12 fusion protein (e.g., SEQ ID NOS: 371, 372, 383)). In some embodiments, the hIL-12 fusion protein mediates from about a 0.5-1000 fold, 0.5-100 fold, 0.5-10 fold, 0.5-5 fold, 0.5-2 fold, 1-1000 fold, 1-100 fold, 1-10 fold, 1-5 fold, 1-2 fold, 10-1000 fold, or 100-1000 fold lower increase in the level of IFN-γ produced by expressing the hIL-12R on the surface, relative to the increase in the level of IFN-γ produced in the presence of a suitable control (e.g., a reference hIL-12 fusion protein (e.g., SEQ ID NOS: 371, 372, 383)). Assays suitable to measure the level of a protein (e.g., IFN-γ) produced from cultured cells are standard and known to the person of ordinary skill in the art, as described, inter alia, in § 5.2.3.

In some embodiments, the hIL-12 fusion protein binds to hIL-12Rβ1 with lower affinity relative to that of a reference hIL-12p40 protein (e.g., a reference hIL-12 fusion protein (e.g., SEQ ID NOS: 371, 372, 383)). Binding affinity can be measured by standard assays known in the art, as described, inter alia, in § 5.2.2.

### 5.3.2 Ig Fusion Proteins & Polypeptides

In some embodiments, the fusion protein comprises one or more hIg heavy chain constant regions (e.g., a CH1 region, a hinge region, a CH2 region, a CH3 region, an Fc region). In some embodiments, the one or more hIg heavy chain constant regions is part of an antibody (e.g., a full-length antibody) (see, e.g., § 5.3.3). In some embodiments, the hIg is a human IgG (hIgG). In some embodiments, the hIgG is hIgG1, IgG2, IgG3, or IgG4. In some embodiments, the hIgG is IgG1 or IgG4. In some embodiments, the hIgG is hIgG1. In some embodiments, the hIgG is hIgG4.

In some embodiments, the fusion protein comprises an Fc region. In some embodiments, the Fc region is part of an antibody. In some embodiments, the Fc region is part of a full-length antibody. In some embodiments, the Fc region comprises or consists of a CH2 region and a CH3 region. In some embodiments, the Fc region comprises or consists of at least a portion of a hinge region, a CH2 region, and a CH3 region. In some embodiments, the Fc region comprises or consists of a hinge region, a CH2 region, and a CH3 region. In some embodiments, the Fc region comprises or consists of at least a portion of a hIgG CH2 region and a hIgG CH3 region. In some embodiments, the Fc region comprises or consists of at least a portion of a hIgG hinge region, a hIgG CH2 region, and a hIgG CH3 region. In some embodiments, the Fc region comprises or consists of a hIgG hinge region, a hIgG CH2 region, and a hIgG CH3 region. In some embodiments, the Fc region comprises or consists of a hIgG1 CH2 region and a hIgG1 CH3 region. In some embodiments, the Fc region comprises or consists of at least a portion of a hIgG1 hinge region, a hIgG1 CH2 region, and a hIgG1 CH3 region. In some embodiments, the Fc region comprises or consists of a hIgG1 hinge region, a hIgG1 CH2 region, and a hIgG1 CH3 region. In some embodiments, the Fc region comprises or consists of at a hIgG4 CH2 region and a hIgG4 CH3 region. In some embodiments, the Fc region comprises or consists of at least a portion of a hIgG4 hinge region, a hIgG4 CH2 region, and a hIgG4 CH3 region. In some embodiments, the Fc region comprises or consists of a hIgG4 hinge region, a hIgG4 CH2 region, and a hIgG4 CH3 region.

The amino acid sequence of exemplary reference hIgG1 and hIgG4 heavy chain constant regions and light chain constant regions, which can be incorporated in one or more of the embodiments described herein (e.g., a hIL-12 fusion protein described herein (e.g., an anti-CAIX antibody (e.g., a full-length antibody) hIL-12 fusion protein described herein) (or one or more polypeptide thereof)), is provided in Table 12.

TABLE 12
The Amino Acid Sequence of Exemplary hIg Heavy Chain and Light Chain
Constant Region components

| | | SEQ |
|---|---|---|
| Description | Amino Acid Sequence | ID NO |
| hIgG1 CH1 Region | ASTKGPSVFPLAPSSKSTSGGTAALGCLVKDYFPEPVTVSWNS | 119 |
| | GALTSGVHTFPAVLQSSGLYSLSSVVTVPSSSLGTQTYICNVN | |
| | HKPSNTKVDKKV | |
| hIgG1 Hinge Region | EPKSCDKTHTCP | 120 |
| hIgG1 CH2 Region | PCPAPELLGGPSVFLFPPKPKDTLMISRTPEVTCVVVDVSHED | 121 |
| | PEVKFNWYVDGVEVHNAKTKPREEQYNSTYRVVSVLTVLHQDW | |
| | LNGKEYKCKVSNKALPAPIEKTISKAK | |
| hIgG1 CH3 Region | GQPREPQVYTLPPSRDELTKNQVSLTCLVKGFYPSDIAVEWES | 122 |
| With C-terminal Lysine | NGQPENNYKTTPPVLDSDGSFFLYSKLTVDKSRWQQGNVFSCS | |
| | VMHEALHNHYTQKSLSLSPGK | |
| hIgG1 CH3 Region | GQPREPQVYTLPPSRDELTKNQVSLTCLVKGFYPSDIAVEWES | 123 |
| Without C-terminal | NGQPENNYKTTPPVLDSDGSFFLYSKLTVDKSRWQQGNVFSCS | |
| Lysine | VMHEALHNHYTQKSLSLSPG | |
| hIgG1 CH2 Region + | PCPAPELLGGPSVFLFPPKPKDTLMISRTPEVTCVVVDVSHED | 124 |
| CH3 Region | PEVKFNWYVDGVEVHNAKTKPREEQYNSTYRVVSVLTVLHQDW | |
| With C-terminal Lysine | LNGKEYKCKVSNKALPAPIEKTISKAKGQPREPQVYTLPPSRD | |
| | ELTKNQVSLTCLVKGFYPSDIAVEWESNGQPENNYKTTPPVLD | |
| | SDGSFFLYSKLTVDKSRWQQGNVFSCSVMHEALHNHYTQKSLS | |
| | LSPGK | |
| hIgG1 CH2 Region + | PCPAPELLGGPSVFLFPPKPKDTLMISRTPEVTCVVVDVSHED | 125 |
| CH3 Region | PEVKFNWYVDGVEVHNAKTKPREEQYNSTYRVVSVLTVLHQDW | |
| Without C-terminal | LNGKEYKCKVSNKALPAPIEKTISKAKGQPREPQVYTLPPSRD | |
| Lysine | ELTKNQVSLTCLVKGFYPSDIAVEWESNGQPENNYKTTPPVLD | |
| | SDGSFFLYSKLTVDKSRWQQGNVFSCSVMHEALHNHYTQKSLS | |
| | LSPG | |
| hIgG1 Partial Hinge | TCPPCPAPELLGGPSVFLFPPKPKDTLMISRTPEVTCVVVDVS | 126 |
| Region + CH2 Region + | HEDPEVKFNWYVDGVEVHNAKTKPREEQYNSTYRVVSVLTVLH | |
| CH3 Region | QDWLNGKEYKCKVSNKALPAPIEKTISKAKGQPREPQVYTLPP | |
| With C-terminal Lysine | SRDELTKNQVSLTCLVKGFYPSDIAVEWESNGQPENNYKTTPP | |
| | VLDSDGSFFLYSKLTVDKSRWQQGNVFSCSVMHEALHNHYTQK | |
| | SLSLSPGK | |
| hIgG1 Partial Hinge | TCPPCPAPELLGGPSVFLFPPKPKDTLMISRTPEVTCVVVDVS | 127 |
| Region + CH2 Region + | HEDPEVKFNWYVDGVEVHNAKTKPREEQYNSTYRVVSVLTVLH | |
| CH3 Region | QDWLNGKEYKCKVSNKALPAPIEKTISKAKGQPREPQVYTLPP | |
| Without C-terminal | SRDELTKNQVSLTCLVKGFYPSDIAVEWESNGQPENNYKTTPP | |
| Lysine | VLDSDGSFFLYSKLTVDKSRWQQGNVFSCSVMHEALHNHYTQK | |
| | SLSLSPG | |
| hIgG1 Hinge Region + | EPKSCDKTHTCPPCPAPELLGGPSVFLFPPKPKDTLMISRTPE | 128 |
| CH2 Region + CH3 | VTCVVVDVSHEDPEVKFNWYVDGVEVHNAKTKPREEQYNSTYR | |
| Region | VVSVLTVLHQDWLNGKEYKCKVSNKALPAPIEKTISKAKGQPR | |
| With C-terminal Lysine | EPQVYTLPPSRDELTKNQVSLTCLVKGFYPSDIAVEWESNGQP | |
| | ENNYKTTPPVLDSDGSFFLYSKLTVDKSRWQQGNVFSCSVMHE | |
| | ALHNHYTQKSLSLSPGK | |
| hIgG1 Hinge Region + | EPKSCDKTHTCPPCPAPELLGGPSVFLFPPKPKDTLMISRTPE | 129 |
| CH2 Region + CH3 | VTCVVVDVSHEDPEVKFNWYVDGVEVHNAKTKPREEQYNSTYR | |
| Region | VVSVLTVLHQDWLNGKEYKCKVSNKALPAPIEKTISKAKGQPR | |
| Without C-terminal | EPQVYTLPPSRDELTKNQVSLTCLVKGFYPSDIAVEWESNGQP | |
| Lysine | ENNYKTTPPVLDSDGSFFLYSKLTVDKSRWQQGNVFSCSVMHE | |
| | ALHNHYTQKSLSLSPG | |
| hIgG1 CH1+ Hinge | ASTKGPSVFPLAPSSKSTSGGTAALGCLVKDYFPEPVTVSWNS | 130 |
| Region + CH2 Region + | GALTSGVHTFPAVLQSSGLYSLSSVVTVPSSSLGTQTYICNVN | |
| CH3 Region | HKPSNTKVDKKVEPKSCDKTHTCPPCPAPELLGGPSVFLFPPK | |
| With C-terminal Lysine | PKDTLMISRTPEVTCVVVDVSHEDPEVKFNWYVDGVEVHNAKT | |
| | KPREEQYNSTYRVVSVLTVLHQDWLNGKEYKCKVSNKALPAPI | |
| | EKTISKAKGQPREPQVYTLPPSRDELTKNQVSLTCLVKGFYPS | |
| | DIAVEWESNGQPENNYKTTPPVLDSDGSFFLYSKLTVDKSRWQ | |
| | QGNVFSCSVMHEALHNHYTQKSLSLSPGK | |
| hIgG1 CH1 + Hinge | ASTKGPSVFPLAPSSKSTSGGTAALGCLVKDYFPEPVTVSWNS | 131 |
| Region + CH2 Region + | GALTSGVHTFPAVLQSSGLYSLSSVVTVPSSSLGTQTYICNVN | |
| CH3 Region | HKPSNTKVDKKVEPKSCDKTHTCPPCPAPELLGGPSVFLFPPK | |
| Without C-terminal | PKDTLMISRTPEVTCVVVDVSHEDPEVKFNWYVDGVEVHNAKT | |
| Lysine | KPREEQYNSTYRVVSVLTVLHQDWLNGKEYKCKVSNKALPAPI | |
| | EKTISKAKGQPREPQVYTLPPSRDELTKNQVSLTCLVKGFYPS | |
| | DIAVEWESNGQPENNYKTTPPVLDSDGSFFLYSKLTVDKSRWQ | |
| | QGNVFSCSVMHEALHNHYTQKSLSLSPG | |
| hIgG4 CH1 Region | ASTKGPSVFPLAPCSRSTSESTAALGCLVKDYFPEPVTVSWNS | 132 |
| | GALTSGVHTFPAVLQSSGLYSLSSVVTVPSSSLGTKTYTCNVD | |
| | HKPSNTKVDKRV | |
| hIgG4 Hinge Region | ESKYGPPCPSCP | 133 |
| hIgG4 CH2 Region | APEFLGGPSVFLFPPKPKDTLMISRTPEVTCVVVDVSQEDPEV | 134 |
| | QFNWYVDGVEVHNAKTKPREEQFNSTYRVVSVLTVLHQDWLNG | |
| | KEYKCKVSNKGLPSSIEKTISKAK | |
| hIgG4 CH3 Region | GQPREPQVYTLPPSQEEMTKNQVSLTCLVKGFYPSDIAVEWES | 135 |
| With C-terminal Lysine | NGQPENNYKTTPPVLDSDGSFFLYSRLTVDKSRWQEGNVFSCS | |
| | VMHEALHNHYTQKSLSLSLGK | |
| hIgG4 CH3 Region | GQPREPQVYTLPPSQEEMTKNQVSLTCLVKGFYPSDIAVEWES | 136 |
| Without C-terminal | NGQPENNYKTTPPVLDSDGSFFLYSRLTVDKSRWQEGNVFSCS | |
| Lysine | VMHEALHNHYTQKSLSLSLG | |
| hIgG4 CH2 Region + | APEFLGGPSVFLFPPKPKDTLMISRTPEVTCVVVDVSQEDPEV | 137 |
| CH3 Region | QFNWYVDGVEVHNAKTKPREEQFNSTYRVVSVLTVLHQDWLNG | |
| With C-terminal Lysine | KEYKCKVSNKGLPSSIEKTISKAKGQPREPQVYTLPPSQEEMT | |
| | KNQVSLTCLVKGFYPSDIAVEWESNGQPENNYKTTPPVLDSDG | |
| | SFFLYSRLTVDKSRWQEGNVFSCSVMHEALHNHYTQKSLSLSL | |
| | GK | |
| hIgG4 CH2 Region + | APEFLGGPSVFLFPPKPKDTLMISRTPEVTCVVVDVSQEDPEV | 138 |
| CH3 Region | QFNWYVDGVEVHNAKTKPREEQFNSTYRVVSVLTVLHQDWLNG | |
| Without C-terminal | KEYKCKVSNKGLPSSIEKTISKAKGQPREPQVYTLPPSQEEMT | |
| Lysine | KNQVSLTCLVKGFYPSDIAVEWESNGQPENNYKTTPPVLDSDG | |
| | SFFLYSRLTVDKSRWQEGNVFSCSVMHEALHNHYTQKSLSLSL | |
| | G | |
| hIgG4 Partial Hinge | PCPSCPAPEFLGGPSVFLFPPKPKDTLMISRTPEVTCVVVDVS | 139 |
| Region + CH2 Region + | QEDPEVQFNWYVDGVEVHNAKTKPREEQFNSTYRVVSVLTVLH | |
| CH3 Region | QDWLNGKEYKCKVSNKGLPSSIEKTISKAKGQPREPQVYTLPP | |
| With C-terminal Lysine | SQEEMTKNQVSLTCLVKGFYPSDIAVEWESNGQPENNYKTTPP | |
| | VLDSDGSFFLYSRLTVDKSRWQEGNVFSCSVMHEALHNHYTQK | |
| | SLSLSLGK | |
| hIgG4 Partial Hinge | PCPSCPAPEFLGGPSVFLFPPKPKDTLMISRTPEVTCVVVDVS | 140 |
| Region + CH2 Region + | QEDPEVQFNWYVDGVEVHNAKTKPREEQFNSTYRVVSVLTVLH | |
| CH3 Region | QDWLNGKEYKCKVSNKGLPSSIEKTISKAKGQPREPQVYTLPP | |
| Without C-terminal | SQEEMTKNQVSLTCLVKGFYPSDIAVEWESNGQPENNYKTTPP | |
| Lysine | VLDSDGSFFLYSRLTVDKSRWQEGNVFSCSVMHEALHNHYTQK | |
| | SLSLSLG | |
| hIgG4 Hinge Region + | ESKYGPPCPSCPAPEFLGGPSVFLFPPKPKDTLMISRTPEVTC | 141 |
| CH2 Region + CH3 | VVVDVSQEDPEVQFNWYVDGVEVHNAKTKPREEQENSTYRVVS | |
| Region | VLTVLHQDWLNGKEYKCKVSNKGLPSSIEKTISKAKGQPREPQ | |
| With C-terminal Lysine | VYTLPPSQEEMTKNQVSLTCLVKGFYPSDIAVEWESNGQPENN | |
| | YKTTPPVLDSDGSFFLYSRLTVDKSRWQEGNVFSCSVMHEALH | |
| | NHYTQKSLSLSLGK | |
| hIgG4 Hinge Region + | ESKYGPPCPSCPAPEFLGGPSVFLFPPKPKDTLMISRTPEVTC | 142 |
| CH2 Region + CH3 | VVVDVSQEDPEVQFNWYVDGVEVHNAKTKPREEQFNSTYRVVS | |
| Region | VLTVLHQDWLNGKEYKCKVSNKGLPSSIEKTISKAKGQPREPQ | |
| Without C-terminal | VYTLPPSQEEMTKNQVSLTCLVKGFYPSDIAVEWESNGQPENN | |
| Lysine | YKTTPPVLDSDGSFFLYSRLTVDKSRWQEGNVFSCSVMHEALH | |
| | NHYTQKSLSLSLG | |
| hIgG4 CH1 + Hinge | ASTKGPSVFPLAPCSRSTSESTAALGCLVKDYFPEPVTVSWNS | 143 |
| Region + CH2 Region + | GALTSGVHTFPAVLQSSGLYSLSSVVTVPSSSLGTKTYTCNVD | |
| CH3 Region | HKPSNTKVDKRVESKYGPPCPSCPAPEFLGGPSVFLFPPKPKD | |
| With C-terminal Lysine | TLMISRTPEVTCVVVDVSQEDPEVQFNWYVDGVEVHNAKTKPR | |
| | EEQFNSTYRVVSVLTVLHQDWLNGKEYKCKVSNKGLPSSIEKT | |
| | ISKAKGQPREPQVYTLPPSQEEMTKNQVSLTCLVKGFYPSDIA | |
| | VEWESNGQPENNYKTTPPVLDSDGSFFLYSRLTVDKSRWQEGN | |
| | VFSCSVMHEALHNHYTQKSLSLSLGK | |
| hIgG4 CH1 + Hinge | ASTKGPSVFPLAPCSRSTSESTAALGCLVKDYFPEPVTVSWNS | 144 |
| Region + CH2 Region + | GALTSGVHTFPAVLQSSGLYSLSSVVTVPSSSLGTKTYTCNVD | |
| CH3 Region | HKPSNTKVDKRVESKYGPPCPSCPAPEFLGGPSVFLFPPKPKD | |
| Without C-terminal | TLMISRTPEVTCVVVDVSQEDPEVQFNWYVDGVEVHNAKTKPR | |
| Lysine | EEQFNSTYRVVSVLTVLHQDWLNGKEYKCKVSNKGLPSSIEKT | |
| | ISKAKGQPREPQVYTLPPSQEEMTKNQVSLTCLVKGFYPSDIA | |
| | VEWESNGQPENNYKTTPPVLDSDGSFFLYSRLTVDKSRWQEGN | |
| | VFSCSVMHEALHNHYTQKSLSLSLG | |
| Ig light chain kappa | RTVAAPSVFIFPPSDEQLKSGTASVVCLLNNFYPREAKVQWKV | 145 |
| constant region (KCL) | DNALQSGNSQESVTEQDSKDSTYSLSSTLTLSKADYEKHKVYA | |
| | CEVTHQGLSSPVTKSFNRGEC | |
| Ig light chain kappa | GQPKANPTVTLFPPSSEELQANKATLVCLISDFYPGAVTVAWK | 146 |
| constant region (ACL) | ADGSPVKAGVETTKPSKQSNNKYAASSYLSLTPEQWKSHRSYS | |
| | CQVTHEGSTVEKTVAPTECS | |

In some embodiments, the fusion protein comprises one or more hIg constant region. In some embodiments, the amino acid sequence of the one or more hIg constant region comprises or consists of an amino acid sequence at least 85%, 86%, 87%, 88%, 89%, 90%, 91%, 92%, 93%, 94%, 95%, 96%, 97%, 98%, 99%, or 100% identical to the amino acid sequence of a polypeptide set forth in Table 12.

In some embodiments, the amino acid sequence of the one or more hIg constant region comprises or consists of the amino acid sequence of a polypeptide set forth in Table 12, and further comprises 1 or more but less than 15% (less than 12%, less than 10%, less than 8%), amino acid variations (e.g., amino acid substitutions, deletions, or additions). In some embodiments, the amino acid sequence of the one or more hIg constant region comprises or consists of the amino acid sequence of a polypeptide set forth in Table 12, comprising or consisting of at least about 1, 2, 3, 4, 5, 6, 7, 8, 9, 10, or more amino acid modifications (e.g., amino acid substitutions, deletions, or additions). In some embodiments, the amino acid sequence of the one or more hIg constant region comprises or consists of the amino acid sequence of a polypeptide set forth in Table 12, comprising or consisting of about 1, 2, 3, 4, 5, 6, 7, 8, 9, 10, or more amino acid modifications (e.g., amino acid substitutions, deletions, or additions). In some embodiments, the amino acid sequence of the one or more hIg constant region comprises or consists of the amino acid sequence of a polypeptide set forth in Table 12, comprising or consisting of about no more than 1, 2, 3, 4, 5, 6, 7, 8, 9, 10, or more amino acid modifications (e.g., amino acid substitutions, deletions, or additions).

In some embodiments, the amino acid sequence of the one or more hIg constant region comprises or consists of an amino acid sequence of a polypeptide set forth in Table 12, and further comprises 1 or more but less than 15% (less than 12%, less than 10%, less than 8%), amino acid substitutions. In some embodiments, the amino acid sequence of the one or more hIg constant region comprises or consists of an amino acid sequence of a polypeptide set forth in Table 12, comprising or consisting of at least about 1, 2, 3, 4, 5, 6, 7, 8, 9, 10, or more amino acid substitutions. In some embodiments, the amino acid sequence of the one or more hIg constant region comprises or consists of an amino acid sequence of a polypeptide set forth in Table 12, comprising or consisting of about 1, 2, 3, 4, 5, 6, 7, 8, 9, 10, or more amino acid substitutions. In some embodiments, the amino acid sequence of the one or more hIg constant region comprises or consists of an amino acid sequence of a polypeptide set forth in Table 12, comprising or consisting of about no more than 1, 2, 3, 4, 5, 6, 7, 8, 9, 10, or more amino acid substitutions.

In some embodiments, the amino acid sequence of the one or more hIg constant region comprises or consists of an amino acid sequence at least 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 119-146 (e.g., any one of SEQ ID NOS: 124-131 or 137-144).

In some embodiments, the amino acid sequence of the one or more hIg constant region comprises or consists of the amino acid sequence set forth in any one of SEQ ID NOS: 119-146 (e.g., any one of SEQ ID NOS: 124-131 or 137-144), and further comprises 1 or more but less than 15% (less than 12%, less than 10%, less than 8%), amino acid modifications (e.g., amino acid substitutions, deletions, or additions). In some embodiments, the amino acid sequence of the one or more hIg constant region comprises or consists of the amino acid sequence set forth in any one of SEQ ID NOS: 119-146 (e.g., any one of SEQ ID NOS: 124-131 or 137-144), comprising or consisting at least about 1, 2, 3, 4, 5, 6, 7, 8, 9, 10, or more amino acid modifications (e.g., amino acid substitutions, deletions, or additions). In some embodiments, the amino acid sequence of the one or more hIg constant region comprises or consists of the amino acid sequence set forth in any one of SEQ ID NOS: 119-146 (e.g., any one of SEQ ID NOS: 124-131 or 137-144), comprising or consisting about 1, 2, 3, 4, 5, 6, 7, 8, 9, or 10 amino acid modifications (e.g., amino acid substitutions, deletions, or additions). In some embodiments, the amino acid sequence of the one or more hIg constant region comprises or consists of the amino acid sequence set forth in any one of SEQ ID NOS: 119-146 (e.g., any one of SEQ ID NOS: 124-131 or 137-144), comprising or consisting of no more than about 1, 2, 3, 4, 5, 6, 7, 8, 9, or 10 amino acid modifications (e.g., amino acid substitutions, deletions, or additions).

In some embodiments, the amino acid sequence of the one or more hIg constant region comprises or consists of the amino acid sequence set forth in any one of SEQ ID NOS: 119-146 (e.g., any one of SEQ ID NOS: 124-131 or 137-144), and further comprises 1 or more but less than 15% (less than 12%, less than 10%, less than 8%), amino acid substitutions. In some embodiments, the amino acid sequence of the one or more hIg constant region comprises or consists of the amino acid sequence set forth in any one of SEQ ID NOS: 119-146 (e.g., any one of SEQ ID NOS: 124-131 or 137-144), comprising or consisting at least about 1, 2, 3, 4, 5, 6, 7, 8, 9, 10, or more amino acid substitutions. In some embodiments, the amino acid sequence of the one or more hIg constant region comprises or consists of the amino acid sequence set forth in any one of SEQ ID NOS: 119-146 (e.g., any one of SEQ ID NOS: 124-131 or 137-144), comprising or consisting about 1, 2, 3, 4, 5, 6, 7, 8, 9, or 10 amino acid substitutions. In some embodiments, the amino acid sequence of the one or more hIg constant region comprises or consists of the amino acid sequence set forth in any one of SEQ ID NOS: 119-146 (e.g., any one of SEQ ID NOS: 124-131 or 137-144), comprising or consisting of no more than about 1, 2, 3, 4, 5, 6, 7, 8, 9, or 10 amino acid substitutions.

In some embodiments, the fusion protein comprises a light chain comprising an amino acid sequence at least 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 145-146.

### 5.3.2.1 Ig Effector Function

As described herein, in some embodiments, the fusion protein comprises an Fc region. In some embodiments, the Fc region of a fusion protein or polypeptide described herein exhibits a decrease in one or more Fc effector function relative to a reference (e.g., wild type) Fc region. Exemplary Fc effector functions include, but are not limited to, antibody dependent cellular cytotoxicity (ADCC), antibody dependent cellular phagocytosis (ADCP), complement dependent cytotoxicity (CDC), and binding affinity to one or more human Fc receptor (e.g., an Fcγ receptor (e.g., FcγRI, FcγRIIa, FcγRIIc, FcγRIIIa, and/or FcγRIIIb (e.g., FcγRI, FcγIIa, and/or FcγIIIa))).

Standard in vitro and/or in vitro assays known in the art can be conducted to evaluate Fc effector function, including, any one or more of ADCC, CDC, ADCP, Fc receptor (e.g., Fcγ receptor) binding affinity, and Clq binding affinity.

For example, ADCC activity can be assessed utilizing standard (radioactive and non-radioactive) methods known in the art (see, e.g., WO2006/082515, WO2012/130831), the entire contents of each of which is incorporated herein by reference for all purposes). For example, ADCC activity can be assessed using a chromium-5 (⁵¹Cr) assay. Briefly, ⁵¹Cr is pre-loaded into target cells expressing CD20, NK cells are added to the culture, and radioactivity in the cell culture supernatant is assessed (indicative of lysis of the target cells by the NK cells). Similar non-radioactive assays can also be utilized that employ a similar method, but the target cells are pre-loaded with fluorescent dyes, such as calcein-AM, CFSE, BCECF, or lanthanide flurophore (Europium). See, e.g., Parekh, Bhavin S et al. “Development and validation of an antibody-dependent cell-mediated cytotoxicity-reporter gene assay.” mAbs vol. 4,3 (2012): 310-8. Doi: 10.4161/mabs.19873, the entire contents of which is incorporated herein by reference for all purposes. Exemplary commercially available non-radioactive assays include, for example, ACTI™ non-radioactive cytotoxicity assay for flow cytometry (Cell Technology, Inc. Mountain View, Calif.; and CytoTox 96® non-radioactive cytotoxicity assay (Promega, Madison, Wis.). Additional non-limiting examples of in vitro assays that can be used to assess ADCC activity of a fusion protein described herein include those described in U.S. Pat. Nos. 5,500,362; 5,821,337; Hellstrom, I., et al., Proc. Nat'l Acad. Sci. USA 83 (1986) 7059-7063; Hellstrom, I., et al., Proc. Nat'l Acad. Sci. USA 82 (1985) 1499-1502; and Bruggemann, M., et al., J. Exp. Med. 166 (1987) 1351-1361, the entire contents of each of which is incorporated herein by reference. Alternatively, or additionally, ADCC activity of a fusion protein described herein may be assessed in vitro, e.g., in an animal model such as that disclosed in Clynes, et al., Proc. Nat'l Acad. Sci. USA 95 (1998) 652-656, the entire contents of which is incorporated herein by reference for all purposes.

Clq binding assays can be utilized to assess the ability of a hIg fusion protein or polypeptide described herein to bind Clq (or bind with less affinity than a reference fusion protein) and hence lack (or have decreased) CDC activity. The binding of a hIg fusion protein or polypeptide described herein to Clq can be determined by a variety of in vitro assays (e.g., biochemical or immunological based assays) known in the art for determining Fc-Clq interactions, including e.g., equilibrium methods (e.g., enzyme-linked immunosorbent assay (ELISA) or radioimmunoassay (RIA)), or kinetic methods (e.g., surface plasmon resonance (SPR) analysis), and other methods such as indirect binding assays, competitive inhibition assays, fluorescence resonance energy transfer (FRET), gel electrophoresis, and chromatography (e.g., gel filtration). These and other methods may utilize a label on one or more of the components being examined and/or employ a variety of detection methods including but not limited to chromogenic, fluorescent, luminescent, or isotopic labels. A detailed description of binding affinities and kinetics can be found in e.g., Paul, W. E., ed., Fundamental Immunology, 4^{th} Ed., Lippincott-Raven, Philadelphia (1999), the entire contents of which is incorporated herein by reference. For example, see, e.g., Clq and C3c binding ELISAs described in WO2006/029879 and WO2005/100402, the entire contents of each of which is incorporated herein by reference for all purposes. Additional CDC activity assays include those described in e.g., Gazzano-Santoro, et al., J. Immunol. Methods 202 (1996) 163; Cragg, M. S., et al., Blood 101 (2003) 1045-1052; and Cragg, M. S., and Glennie, M. J., Blood 103 (2004) 2738-2743), the entire contents of each of which is incorporated herein by reference for all purposes.

ADCP activity can be measured by in vitro or in vitro methods known in the art and also commercially available assays (see, e.g., van de Donk N W, Moreau P, Plesner T, et al. “Clinical efficacy and management of monoclonal antibodies targeting CD38 and SLAMF7 in multiple myeloma,” Blood, 127 (6): 681-695 (2016), the entire contents of each of which is incorporated herein by reference for all purposes). For example, a primary cell based ADCP assay can be used in which fresh human peripheral blood mononuclear cells (PBMCs) are isolated, monocytes isolated and differentiated in culture to macrophages using standard procedures. The macrophages are fluorescently labeled added to cultures containing fluorescently labeled target cells expressing CD20 and a fusion protein described herein. Phagocytosis events can be analyzed using FACS screening and/or microscopy. A modified reporter version of the above described assay can also be used that employs an engineered cell line that stably expresses FcγRIIa (CD32a) as the effector cell line (e.g., an engineered T cell line, e.g., THP-1), removing the requirement for primary cells. Exemplary ADCP assays are described in e.g., Ackerman, M. E. et al. A robust, high-throughput assay to determine the phagocytic activity of clinical antibody samples. J. Immunol. Methods 366, 8-19 (2011); and Mcandrew, E. G. et al. Determining the phagocytic activity of clinical antibody samples. J. Vis. Exp. 3588 (2011). Doi: 10.3791/3588; the entire contents of each of which is incorporated herein by reference.

Binding of a hIg fusion protein or polypeptide described herein to a Fc receptor can be determined by a variety of in vitro assays (e.g., biochemical or immunological based assays) known in the art for determining Fc-Fc receptor interactions, i.e., specific binding of an Fc region to an Fc receptor. Common assays include equilibrium methods (e.g., enzyme-linked immunosorbent assay (ELISA) or radioimmunoassay (RIA)), or kinetic methods (e.g., surface plasmon resonance (SPR) analysis), and other methods such as indirect binding assays, competitive inhibition assays, fluorescence resonance energy transfer (FRET), gel electrophoresis, and chromatography (e.g., gel filtration). These and other methods may utilize a label on one or more of the components being examined and/or employ a variety of detection methods including but not limited to chromogenic, fluorescent, luminescent, or isotopic labels. A detailed description of binding affinities and kinetics can be found in e.g., Paul, W. E., ed., Fundamental Immunology, 4″ Ed., Lippincott-Raven, Philadelphia (1999), the entire contents of which is incorporated herein by reference for all purposes.

In some embodiments, the Fc region of a fusion protein (or one or more polypeptide thereof) described herein is modified (e.g., comprises one or more amino acid modification (e.g., one or more amino acid substitution, deletion, addition, etc.)) (referred to herein as a “modified Fc region”), relative to the amino acid sequence of a reference Fc region (e.g., a wild type Fc region, e.g., any one of SEQ ID NOS: 124-131). In some embodiments, the one or more amino acid modification (e.g., the one or more amino acid substitution, deletion, addition, etc.)) decreases or abolishes one or more Fc effector function, relative to a reference Fc that does not comprise the modification (e.g., the one or more modification (e.g., the one or more amino acid substitution, deletion, addition, etc.)).

In some embodiments, the fusion protein (or one or more polypeptide thereof) comprising a modified Fc region exhibits no detectable or decreased ADCC compared to a reference fusion protein (or one or more polypeptide thereof) that does not comprise the Fc region modification (e.g., the one or more amino acid modification (e.g., one or more amino acid substitution, deletion, or addition)). In some embodiments, the fusion protein (or one or more polypeptide thereof) comprising a modified Fc region exhibits no detectable or decreased CDC compared to a reference fusion protein (or one or more polypeptide thereof) that does not comprise the Fc region modification (e.g., the one or more amino acid modification (e.g., one or more amino acid substitution, deletion, or addition)). In some embodiments, the fusion protein (or one or more polypeptide thereof) comprising a modified Fc region exhibits no detectable or decreased ADCP compared to a reference fusion protein (or one or more polypeptide thereof) that does not comprise the Fc region modification (e.g., the one or more modification (e.g., one or more amino acid substitution, deletion, or addition)). In some embodiments, the fusion protein (or one or more polypeptide thereof) comprising a modified Fc region exhibits decreased or no detectable specific binding affinity to one or more human Fc receptor (e.g., an Fcγ receptor (e.g., FcγRI, FcγRIIa, FcγRIIc, FcγRIIIa, and/or FcγRIIIb (e.g., FcγRI, FcγIIa, and/or FcγIIIa))) compared to a reference fusion protein (or one or more polypeptide thereof) that does not comprise the Fc region modification (e.g., the one or more modification (e.g., one or more amino acid substitution, deletion, or addition)). In some embodiments, the fusion protein (or one or more polypeptide thereof) comprising a modified Fc region exhibits decreased or no detectable specific binding affinity to FcγRI, FcγIIa, and/or FcγIIIa compared to a reference fusion protein (or one or more polypeptide thereof) that does not comprise the Fc region modification (e.g., the one or more modification (e.g., one or more amino acid substitution, deletion, or addition)). In some embodiments, the fusion protein (or polypeptide thereof) or polypeptide comprising a modified Fc exhibits decreased or no detectable specific binding affinity to FcγRI compared to a reference fusion protein (or one or more polypeptide thereof) that does not comprise the Fc modification (e.g., the one or more modification (e.g., one or more amino acid substitution, deletion, or addition)). In some embodiments, the fusion protein (or one or more polypeptide thereof) comprising a modified Fc region exhibits decreased or no detectable specific binding affinity to FcγIIa compared to a reference fusion protein (or polypeptide thereof) or polypeptide that does not comprise the Fc region modification (e.g., the one or more modification (e.g., one or more amino acid substitution, deletion, or addition)). In some embodiments, the fusion protein (or one or more polypeptide thereof) comprising a modified Fc region exhibits decreased or no detectable specific binding affinity to FcγIIIa compared to a reference fusion protein (or one or more polypeptide thereof) that does not comprise the Fc region modification (e.g., the one or more modification (e.g., one or more amino acid substitution, deletion, or addition)). In some embodiments, the fusion protein (or one or more polypeptide thereof) comprising a modified Fc region exhibits decreased or no detectable specific binding affinity to Clq compared to a reference fusion protein (or one or more polypeptide thereof) that does not comprise the Fc region modification (e.g., the one or more modification (e.g., one or more amino acid substitution, deletion, or addition)).

In some embodiments, the fusion protein (or one or more polypeptide thereof) comprising an Fc region exhibits no detectable ADCC. In some embodiments, the fusion protein (or one or more polypeptide thereof) comprising an Fc region exhibits no detectable CDC. In some embodiments, the fusion protein (or one or more polypeptide thereof) comprising an Fc region exhibits no detectable ADCP. In some embodiments, the fusion protein (or one or more polypeptide thereof) comprising an Fc region exhibits no detectable specific binding affinity to one or more human Fc receptor (e.g., an Fcγ receptor (e.g., FcγRI, FcγRIIa, FcγRIIc, FcγRIIIa, and/or FcγRIIIb (e.g., FcγRI, FcγIIa, and/or FcγIIIa))). In some embodiments, the fusion protein (or one or more polypeptide thereof) comprising an Fc region exhibits no detectable specific binding affinity to FcγRI, FcγIIa, and/or. In some embodiments, the fusion protein (or polypeptide thereof) comprising an Fc exhibits no detectable specific binding affinity to FcγRI. In some embodiments, the fusion protein (or one or more polypeptide thereof) comprising an Fc exhibits no detectable specific binding affinity to FcγIIa. In some embodiments, the fusion protein (or one or more polypeptide thereof) comprising an Fc region exhibits no detectable specific binding affinity to FcγIIIa. In some embodiments, the fusion protein (or one or more polypeptide thereof) comprising an Fc region exhibits no detectable specific binding affinity to Clq.

Amino acid substitutions that decrease or abolish one or more Fc effector function are known in the art. See for example, Saunders Kevin, “Conceptual Approaches to Modulating Antibody Effector Functions and Circulation Half-Life,” Frontiers in Immunology, v10 (Jun. 7, 2019) DOI=10.3389/fimmu.2019.01296, the full contents of which is incorporated herein by reference for all purposes, see more particularly for example, e.g., Table 3 of Saunders.

In some embodiments, the modified Fc comprises a hIgG1 Fc region comprising one or more amino acid modification (e.g., one or more amino acid substitution). In some embodiments, the hIgG1 Fc region comprises an amino acid substitution at amino acid positions L234, L235, and/or P329, EU numbering according to Kabat. In some embodiments, the hIgG1 Fc region comprises the following amino acid substitutions L234A and/or L235A, EU numbering according to Kabat. In some embodiments, the hIgG1 Fc region comprises the following amino acid substitutions L234A, L235A, and P329G, EU numbering according to Kabat. In some embodiments, the hIgG1 Fc region comprises the following amino acid substitutions L234A, L235A, and P329A, EU numbering according to Kabat.

In some embodiments, the modified Fc region comprises a hIgG4 Fc region comprising one or more amino acid modification (e.g., one or more amino acid substitution). In some embodiments, the hIgG4 Fc region comprises an amino acid substitution at amino acid positions S228, F234, and/or L235, EU numbering according to Kabat. In some embodiments, the hIgG4 Fc region comprises the following amino acid substitutions S228P, F234A, and/or L235A, EU numbering according to Kabat. In some embodiments, the hIgG4 Fc region comprises the following amino acid substitutions S228P, F234A, and/or L235E, EU numbering according to Kabat. In some embodiments, the hIgG4 Fc comprises the following amino acid substitutions S228P and/or L235E, EU numbering according to Kabat.

The amino acid sequence of exemplary modified Fc regions that are known in the art to exhibit a decrease in one more effector function is provided in Table 13.

TABLE 13
The Amino Acid Sequence of Exemplary Modified Fc Regions

| | | SEQ |
|---|---|---|
| Description | Amino Acid Sequence | ID NO |
| hIgG1 CH2 Region + CH3 | PCPAPEAAGGPSVFLFPPKPKDTLMISRTPEVTCVVVDVSHE | 147 |
| Region | DPEVKFNWYVDGVEVHNAKTKPREEQYNSTYRVVSVLTVLHQ | |
| L234A/L235A | DWLNGKEYKCKVSNKALPAPIEKTISKAKGQPREPQVYTLPP | |
| With C-terminal Lysine | SRDELTKNQVSLTCLVKGFYPSDIAVEWESNGQPENNYKTTP | |
| | PVLDSDGSFFLYSKLTVDKSRWQQGNVFSCSVMHEALHNHYT | |
| | QKSLSLSPGK | |
| hIgG1 CH2 Region + CH3 | PCPAPEAAGGPSVFLFPPKPKDTLMISRTPEVTCVVVDVSHE | 148 |
| Region | DPEVKFNWYVDGVEVHNAKTKPREEQYNSTYRVVSVLTVLHQ | |
| L234A/L235A | DWLNGKEYKCKVSNKALPAPIEKTISKAKGQPREPQVYTLPP | |
| Without C-terminal Lysine | SRDELTKNQVSLTCLVKGFYPSDIAVEWESNGQPENNYKTTP | |
| | PVLDSDGSFFLYSKLTVDKSRWQQGNVFSCSVMHEALHNHYT | |
| | QKSLSLSPG | |
| hIgG1 CH2 Region + CH3 | PCPAPEAAGGPSVFLFPPKPKDTLMISRTPEVTCVVVDVSHE | 149 |
| Region | DPEVKFNWYVDGVEVHNAKTKPREEQYNSTYRVVSVLTVLHQ | |
| L234A/L235A/P329A | DWLNGKEYKCKVSNKALAAPIEKTISKAKGQPREPQVYTLPP | |
| With C-terminal Lysine | SRDELTKNQVSLTCLVKGFYPSDIAVEWESNGQPENNYKTTP | |
| | PVLDSDGSFFLYSKLTVDKSRWQQGNVFSCSVMHEALHNHYT | |
| | QKSLSLSPGK | |
| hIgG1 CH2 Region + CH3 | PCPAPEAAGGPSVFLFPPKPKDTLMISRTPEVTCVVVDVSHE | 150 |
| Region | DPEVKFNWYVDGVEVHNAKTKPREEQYNSTYRVVSVLTVLHQ | |
| L234A/L235A/P329A | DWLNGKEYKCKVSNKALAAPIEKTISKAKGQPREPQVYTLPP | |
| Without C-terminal Lysine | SRDELTKNQVSLTCLVKGFYPSDIAVEWESNGQPENNYKTTP | |
| | PVLDSDGSFFLYSKLTVDKSRWQQGNVFSCSVMHEALHNHYT | |
| | QKSLSLSPG | |
| hIgG1 CH2 Region + CH3 | PCPAPEAAGGPSVFLFPPKPKDTLMISRTPEVTCVVVDVSHE | 151 |
| Region | DPEVKFNWYVDGVEVHNAKTKPREEQYNSTYRVVSVLTVLHQ | |
| L234A/L235A/P329G | DWLNGKEYKCKVSNKALGAPIEKTISKAKGQPREPQVYTLPP | |
| With C-terminal Lysine | SRDELTKNQVSLTCLVKGFYPSDIAVEWESNGQPENNYKTTP | |
| | PVLDSDGSFFLYSKLTVDKSRWQQGNVFSCSVMHEALHNHYT | |
| | QKSLSLSPGK | |
| hIgG1 CH2 Region + CH3 | PCPAPEAAGGPSVFLFPPKPKDTLMISRTPEVTCVVVDVSHE | 152 |
| Region | DPEVKFNWYVDGVEVHNAKTKPREEQYNSTYRVVSVLTVLHQ | |
| L234A/L235A/P329G | DWLNGKEYKCKVSNKALGAPIEKTISKAKGQPREPQVYTLPP | |
| Without C-terminal Lysine | SRDELTKNQVSLTCLVKGFYPSDIAVEWESNGQPENNYKTTP | |
| | PVLDSDGSFFLYSKLTVDKSRWQQGNVFSCSVMHEALHNHYT | |
| | QKSLSLSPG | |
| hIgG1 Partial Hinge | TCPPCPAPEAAGGPSVFLFPPKPKDTLMISRTPEVTCVVVDV | 153 |
| Region + CH2 Region + CH3 | SHEDPEVKFNWYVDGVEVHNAKTKPREEQYNSTYRVVSVLTV | |
| Region | LHQDWLNGKEYKCKVSNKALPAPIEKTISKAKGQPREPQVYT | |
| L234A/L235A | LPPSRDELTKNQVSLTCLVKGFYPSDIAVEWESNGQPENNYK | |
| With C-terminal Lysine | TTPPVLDSDGSFFLYSKLTVDKSRWQQGNVFSCSVMHEALHN | |
| | HYTQKSLSLSPGK | |
| hIgG1 Partial Hinge | TCPPCPAPEAAGGPSVFLFPPKPKDTLMISRTPEVTCVVVDV | 154 |
| Region + CH2 Region + CH3 | SHEDPEVKFNWYVDGVEVHNAKTKPREEQYNSTYRVVSVLTV | |
| Region | LHQDWLNGKEYKCKVSNKALPAPIEKTISKAKGQPREPQVYT | |
| L234A/L235A | LPPSRDELTKNQVSLTCLVKGFYPSDIAVEWESNGQPENNYK | |
| Without C-terminal Lysine | TTPPVLDSDGSFFLYSKLTVDKSRWQQGNVFSCSVMHEALHN | |
| | HYTQKSLSLSPG | |
| hIgG1 Partial Hinge | TCPPCPAPEAAGGPSVFLFPPKPKDTLMISRTPEVTCVVVDV | 155 |
| Region + CH2 Region + CH3 | SHEDPEVKFNWYVDGVEVHNAKTKPREEQYNSTYRVVSVLTV | |
| Region | LHQDWLNGKEYKCKVSNKALAAPIEKTISKAKGQPREPQVYT | |
| L234A/L235A/P329A | LPPSRDELTKNQVSLTCLVKGFYPSDIAVEWESNGQPENNYK | |
| With C-terminal Lysine | TTPPVLDSDGSFFLYSKLTVDKSRWQQGNVFSCSVMHEALHN | |
| | HYTQKSLSLSPGK | |
| hIgG1 Partial Hinge | TCPPCPAPEAAGGPSVFLFPPKPKDTLMISRTPEVTCVVVDV | 156 |
| Region + CH2 Region + CH3 | SHEDPEVKFNWYVDGVEVHNAKTKPREEQYNSTYRVVSVLTV | |
| Region | LHQDWLNGKEYKCKVSNKALAAPIEKTISKAKGQPREPQVYT | |
| L234A/L235A/P329A | LPPSRDELTKNQVSLTCLVKGFYPSDIAVEWESNGQPENNYK | |
| Without C-terminal Lysine | TTPPVLDSDGSFFLYSKLTVDKSRWQQGNVFSCSVMHEALHN | |
| | HYTQKSLSLSPG | |
| hIgG1 Partial Hinge | TCPPCPAPEAAGGPSVFLFPPKPKDTLMISRTPEVTCVVVDV | 157 |
| Region + CH2 Region + CH3 | SHEDPEVKFNWYVDGVEVHNAKTKPREEQYNSTYRVVSVLTV | |
| Region | LHQDWLNGKEYKCKVSNKALGAPIEKTISKAKGQPREPQVYT | |
| L234A/L235A/P329G | LPPSRDELTKNQVSLTCLVKGFYPSDIAVEWESNGQPENNYK | |
| With C-terminal Lysine | TTPPVLDSDGSFFLYSKLTVDKSRWQQGNVFSCSVMHEALHN | |
| | HYTQKSLSLSPGK | |
| hIgG1 Partial Hinge | TCPPCPAPEAAGGPSVFLFPPKPKDTLMISRTPEVTCVVVDV | 158 |
| Region + CH2 Region + CH3 | SHEDPEVKFNWYVDGVEVHNAKTKPREEQYNSTYRVVSVLTV | |
| Region | LHQDWLNGKEYKCKVSNKALGAPIEKTISKAKGQPREPQVYT | |
| L234A/L235A/P329G | LPPSRDELTKNQVSLTCLVKGFYPSDIAVEWESNGQPENNYK | |
| Without C-terminal Lysine | TTPPVLDSDGSFFLYSKLTVDKSRWQQGNVFSCSVMHEALHN | |
| | HYTQKSLSLSPG | |
| hIgG1 Hinge Region + | EPKSCDKTHTCPPCPAPEAAGGPSVFLFPPKPKDTLMISRTP | 159 |
| CH2 Region + CH3 Region | EVTCVVVDVSHEDPEVKFNWYVDGVEVHNAKTKPREEQYNST | |
| L234A/L235A | YRVVSVLTVLHQDWLNGKEYKCKVSNKALPAPIEKTISKAKG | |
| With C-terminal Lysine | QPREPQVYTLPPSRDELTKNQVSLTCLVKGFYPSDIAVEWES | |
| | NGQPENNYKTTPPVLDSDGSFFLYSKLTVDKSRWQQGNVFSC | |
| | SVMHEALHNHYTQKSLSLSPGK | |
| hIgG1 Hinge Region + | EPKSCDKTHTCPPCPAPEAAGGPSVFLFPPKPKDTLMISRTP | 160 |
| CH2 Region + CH3 Region | EVTCVVVDVSHEDPEVKFNWYVDGVEVHNAKTKPREEQYNST | |
| L234A/L235A | YRVVSVLTVLHQDWLNGKEYKCKVSNKALPAPIEKTISKAKG | |
| Without C-terminal Lysine | QPREPQVYTLPPSRDELTKNQVSLTCLVKGFYPSDIAVEWES | |
| | NGQPENNYKTTPPVLDSDGSFFLYSKLTVDKSRWQQGNVFSC | |
| | SVMHEALHNHYTQKSLSLSPG | |
| hIgG1 Hinge Region + | EPKSCDKTHTCPPCPAPEAAGGPSVFLFPPKPKDTLMISRTP | 161 |
| CH2 Region + CH3 Region | EVTCVVVDVSHEDPEVKFNWYVDGVEVHNAKTKPREEQYNST | |
| L234A/L235A/P329A | YRVVSVLTVLHQDWLNGKEYKCKVSNKALPAPIEKTISKAKG | |
| With C-terminal Lysine | QPREPQVYTLPPSRDELTKNQVSLTCLVKGFYPSDIAVEWES | |
| | NGQPENNYKTTPPVLDSDGSFFLYSKLTVDKSRWQQGNVFSC | |
| | SVMHEALHNHYTQKSLSLSPGK | |
| hIgG1 Hinge Region + | EPKSCDKTHTCPPCPAPEAAGGPSVFLFPPKPKDTLMISRTP | 162 |
| CH2 Region + CH3 Region | EVTCVVVDVSHEDPEVKFNWYVDGVEVHNAKTKPREEQYNST | |
| L234A/L235A/P329A | YRVVSVLTVLHQDWLNGKEYKCKVSNKALPAPIEKTISKAKG | |
| Without C-terminal Lysine | QPREPQVYTLPPSRDELTKNQVSLTCLVKGFYPSDIAVEWES | |
| | NGQPENNYKTTPPVLDSDGSFFLYSKLTVDKSRWQQGNVFSC | |
| | SVMHEALHNHYTQKSLSLSPG | |
| hIgG1 Hinge Region + | EPKSCDKTHTCPPCPAPEAAGGPSVFLFPPKPKDTLMISRTP | 163 |
| CH2 Region + CH3 Region | EVTCVVVDVSHEDPEVKFNWYVDGVEVHNAKTKPREEQYNST | |
| L234A/L235A/P329G | YRVVSVLTVLHQDWLNGKEYKCKVSNKALPAPIEKTISKAKG | |
| With C-terminal Lysine | QPREPQVYTLPPSRDELTKNQVSLTCLVKGFYPSDIAVEWES | |
| | NGQPENNYKTTPPVLDSDGSFFLYSKLTVDKSRWQQGNVFSC | |
| | SVMHEALHNHYTQKSLSLSPGK | |
| hIgG1 Hinge Region + | EPKSCDKTHTCPPCPAPEAAGGPSVFLFPPKPKDTLMISRTP | 164 |
| CH2 Region + CH3 Region | EVTCVVVDVSHEDPEVKFNWYVDGVEVHNAKTKPREEQYNST | |
| L234A/L235A/P329G | YRVVSVLTVLHQDWLNGKEYKCKVSNKALPAPIEKTISKAKG | |
| Without C-terminal Lysine | QPREPQVYTLPPSRDELTKNQVSLTCLVKGFYPSDIAVEWES | |
| | NGQPENNYKTTPPVLDSDGSFFLYSKLTVDKSRWQQGNVFSC | |
| | SVMHEALHNHYTQKSLSLSPG | |
| hIgG4 CH2 Region + CH3 | APEAAGGPSVFLFPPKPKDTLMISRTPEVTCVVVDVSQEDPE | 165 |
| Region | VQFNWYVDGVEVHNAKTKPREEQFNSTYRVVSVLTVLHQDWL | |
| S228P/F234A/L235A | NGKEYKCKVSNKGLPSSIEKTISKAKGQPREPQVYTLPPSQE | |
| With C-terminal Lysine | EMTKNQVSLTCLVKGFYPSDIAVEWESNGQPENNYKTTPPVL | |
| | DSDGSFFLYSRLTVDKSRWQEGNVFSCSVMHEALHNHYTQKS | |
| | LSLSLGK | |
| hIgG4 CH2 Region + CH3 | APEAAGGPSVFLFPPKPKDTLMISRTPEVTCVVVDVSQEDPE | 166 |
| Region | VQFNWYVDGVEVHNAKTKPREEQFNSTYRVVSVLTVLHQDWL | |
| S228P/F234A/L235A | NGKEYKCKVSNKGLPSSIEKTISKAKGQPREPQVYTLPPSQE | |
| Without C-terminal Lysine | EMTKNQVSLTCLVKGFYPSDIAVEWESNGQPENNYKTTPPVL | |
| | DSDGSFFLYSRLTVDKSRWQEGNVFSCSVMHEALHNHYTQKS | |
| | LSLSLG | |
| hIgG4 Partial Hinge | PCPSCPAPEAAGGPSVFLFPPKPKDTLMISRTPEVTCVVVDV | 167 |
| Region + CH2 Region + CH3 | SQEDPEVQFNWYVDGVEVHNAKTKPREEQENSTYRVVSVLTV | |
| Region | LHQDWLNGKEYKCKVSNKGLPSSIEKTISKAKGQPREPQVYT | |
| S228P/F234A/L235A | LPPSQEEMTKNQVSLTCLVKGFYPSDIAVEWESNGQPENNYK | |
| With C-terminal Lysine | TTPPVLDSDGSFFLYSRLTVDKSRWQEGNVFSCSVMHEALHN | |
| | HYTQKSLSLSLGK | |
| hIgG4 Partial Hinge | PCPPCPAPEAAGGPSVFLFPPKPKDTLMISRTPEVTCVVVDV | 168 |
| Region + CH2 Region + CH3 | SQEDPEVQFNWYVDGVEVHNAKTKPREEQFNSTYRVVSVLTV | |
| Region | LHQDWLNGKEYKCKVSNKGLPSSIEKTISKAKGQPREPQVYT | |
| S228P/F234A/L235A | LPPSQEEMTKNQVSLTCLVKGFYPSDIAVEWESNGQPENNYK | |
| Without C-terminal Lysine | TTPPVLDSDGSFFLYSRLTVDKSRWQEGNVFSCSVMHEALHN | |
| | HYTQKSLSLSLG | |
| hIgG4 Hinge Region + | ESKYGPPCPPCPAPEAAGGPSVFLFPPKPKDTLMISRTPEVT | 169 |
| CH2 Region + CH3 Region | CVVVDVSQEDPEVQFNWYVDGVEVHNAKTKPREEQFNSTYRV | |
| S228P/F234A/L235A | VSVLTVLHQDWLNGKEYKCKVSNKGLPSSIEKTISKAKGQPR | |
| With C-terminal Lysine | EPQVYTLPPSQEEMTKNQVSLTCLVKGFYPSDIAVEWESNGQ | |
| | PENNYKTTPPVLDSDGSFFLYSRLTVDKSRWQEGNVFSCSVM | |
| | HEALHNHYTQKSLSLSLGK | |
| hIgG4 Hinge Region + | ESKYGPPCPPCPAPEAAGGPSVFLFPPKPKDTLMISRTPEVT | 170 |
| CH2 Region + CH3 Region | CVVVDVSQEDPEVQFNWYVDGVEVHNAKTKPREEQFNSTYRV | |
| S228P/F234A/L235A | VSVLTVLHQDWLNGKEYKCKVSNKGLPSSIEKTISKAKGQPR | |
| Without C-terminal Lysine | EPQVYTLPPSQEEMTKNQVSLTCLVKGFYPSDIAVEWESNGQ | |
| | PENNYKTTPPVLDSDGSFFLYSRLTVDKSRWQEGNVFSCSVM | |
| | HEALHNHYTQKSLSLSLG | |
| hIgG4 Hinge Region + | AESKYGPPCPPCPAPEAAGGPSVFLFPPKPKDTLMISRTPEV | 171 |
| CH2 Region + CH3 Region | TCVVVDVSQEDPEVQFNWYVDGVEVHNAKTKPREEQFNSTYR | |
| (Modified) | VVSVLTVLHQDWLNGKEYKCKVSNKGLPSSIEKTISKAKGQP | |
| S228P/F234A/L235A | REPQVYTLPPSQEEMTKNQVSLTCLVKGFYPSDIAVEWESNG | |
| With C-terminal Lysine | QPENNYKTTPPVLDSDGSFFLYSRLTVDKSRWQEGNVFSCSV | |
| | MHEALHNHYTQKSLSLSLGK | |
| hIgG4 Hinge Region + | AESKYGPPCPPCPAPEAAGGPSVFLFPPKPKDTLMISRTPEV | 172 |
| CH2 Region + CH3 Region | TCVVVDVSQEDPEVQFNWYVDGVEVHNAKTKPREEQFNSTYR | |
| (Modified) | VVSVLTVLHQDWLNGKEYKCKVSNKGLPSSIEKTISKAKGQP | |
| S228P/F234A/L235A | REPQVYTLPPSQEEMTKNQVSLTCLVKGFYPSDIAVEWESNG | |
| Without C-terminal Lysine | QPENNYKTTPPVLDSDGSFFLYSRLTVDKSRWQEGNVFSCSV | |
| | MHEALHNHYTQKSLSLSLG | |

In some embodiments, the amino acid sequence of the hIgG1 Fc region comprises or consists of an alanine amino acid at position L234 and/or an alanine amino acid at position L235, EU numbering according to Kabat. In some embodiments, the amino acid sequence of the hIgG1 Fc region comprises or consists of an alanine amino acid at position L234 and an alanine amino acid at position L235, EU numbering according to Kabat. In some embodiments, the amino acid sequence of the hIgG1 Fc region comprises or consists of an alanine or glycine amino acid at position P329, EU numbering according to Kabat.

In some embodiments, the amino acid sequence of the hIgG1 Fc region comprises or consists of an alanine amino acid at position L234; an alanine amino acid at position L235; and an alanine or glycine amino acid at position P329 EU numbering according to Kabat. In some embodiments, the amino acid sequence of the hIgG1 Fc region comprises or consists of an alanine amino acid at position L234; an alanine amino acid at position L235; and an alanine amino acid at position P329 EU numbering according to Kabat. In some embodiments, the amino acid sequence of the hIgG1 Fc region comprises or consists of an alanine amino acid at position L234; an alanine amino acid at position L235; and a glycine amino acid at position P329 EU numbering according to Kabat.

In some embodiments, the amino acid sequence of the hIgG1 Fc region comprises or consists of an alanine amino acid at position L234, an alanine amino acid at position L235, and/or an alanine or glycine amino acid at position P329, EU numbering according to Kabat; and comprises an amino acid sequence at least 85%, 86%, 87%, 88%, 89%, 90%, 91%, 92%, 93%, 94%, 95%, 96%, 97%, 98%, 99%, or 100% identical to the amino acid sequence of a polypeptide set forth in Table 13. In some embodiments, the amino acid sequence of the hIgG1 Fc region comprises or consists of an alanine amino acid at position L234 and/or an alanine amino acid at position L235, EU numbering according to Kabat; and comprises an amino acid sequence at least 85%, 86%, 87%, 88%, 89%, 90%, 91%, 92%, 93%, 94%, 95%, 96%, 97%, 98%, 99%, or 100% identical to the amino acid sequence of a polypeptide set forth in Table 13. In some embodiments, the amino acid sequence of the hIgG1 Fc region comprises or consists of an alanine amino acid at position L234 and an alanine amino acid at position L235, EU numbering according to Kabat; and comprises an amino acid sequence at least 85%, 86%, 87%, 88%, 89%, 90%, 91%, 92%, 93%, 94%, 95%, 96%, 97%, 98%, 99%, or 100% identical to the amino acid sequence of a polypeptide set forth in Table 13. In some embodiments, the amino acid sequence of the hIgG1 Fc region comprises or consists of an alanine amino acid at position L234, an alanine amino acid at position L235, and an alanine or glycine amino acid at position P329, EU numbering according to Kabat; and comprises an amino acid sequence at least 85%, 86%, 87%, 88%, 89%, 90%, 91%, 92%, 93%, 94%, 95%, 96%, 97%, 98%, 99%, or 100% identical to the amino acid sequence of a polypeptide set forth in Table 13. In some embodiments, the amino acid sequence of the hIgG1 Fc region comprises or consists of an alanine amino acid at position L234, an alanine amino acid at position L235, and an alanine amino acid at position P329, EU numbering according to Kabat; and comprises an amino acid sequence at least 85%, 86%, 87%, 88%, 89%, 90%, 91%, 92%, 93%, 94%, 95%, 96%, 97%, 98%, 99%, or 100% identical to the amino acid sequence of a polypeptide set forth in Table 13. In some embodiments, the amino acid sequence of the hIgG1 Fc region comprises or consists of an alanine amino acid at position L234, an alanine amino acid at position L235, and a glycine amino acid at position P329, EU numbering according to Kabat; and comprises an amino acid sequence at least 85%, 86%, 87%, 88%, 89%, 90%, 91%, 92%, 93%, 94%, 95%, 96%, 97%, 98%, 99%, or 100% identical to the amino acid sequence of a polypeptide set forth in Table 13.

In some embodiments, the amino acid sequence of the hIgG1 Fc region comprises or consists of an alanine amino acid at position L234, an alanine amino acid at position L235, and/or an alanine or glycine amino acid at position P329, EU numbering according to Kabat; and comprises an amino acid sequence at least 85%, 86%, 87%, 88%, 89%, 90%, 91%, 92%, 93%, 94%, 95%, 96%, 97%, 98%, 99%, or 100% identical to the amino acid sequence of any one of SEQ ID NOS: 147-163. In some embodiments, the amino acid sequence of the hIgG1 Fc region comprises or consists of an alanine amino acid at position L234 and/or an alanine amino acid at position L235, EU numbering according to Kabat; and comprises an amino acid sequence at least 85%, 86%, 87%, 88%, 89%, 90%, 91%, 92%, 93%, 94%, 95%, 96%, 97%, 98%, 99%, or 100% identical to the amino acid sequence of any one of SEQ ID NOS: 147-163. In some embodiments, the amino acid sequence of the hIgG1 Fc region comprises or consists of an alanine amino acid at position L234 and an alanine amino acid at position L235, EU numbering according to Kabat; and comprises an amino acid sequence at least 85%, 86%, 87%, 88%, 89%, 90%, 91%, 92%, 93%, 94%, 95%, 96%, 97%, 98%, 99%, or 100% identical to the amino acid sequence of any one of SEQ ID NOS: 147-163. In some embodiments, the amino acid sequence of the hIgG1 Fc region comprises or consists of an alanine amino acid at position L234, an alanine amino acid at position L235, and an alanine or glycine amino acid at position P329, EU numbering according to Kabat; and comprises an amino acid sequence at least 85%, 86%, 87%, 88%, 89%, 90%, 91%, 92%, 93%, 94%, 95%, 96%, 97%, 98%, 99%, or 100% identical to the amino acid sequence of any one of SEQ ID NOS: 147-163. In some embodiments, the amino acid sequence of the hIgG1 Fc region comprises or consists of an alanine amino acid at position L234, an alanine amino acid at position L235, and an alanine amino acid at position P329, EU numbering according to Kabat; and comprises an amino acid sequence at least 85%, 86%, 87%, 88%, 89%, 90%, 91%, 92%, 93%, 94%, 95%, 96%, 97%, 98%, 99%, or 100% identical to the amino acid sequence of any one of SEQ ID NOS: 147-163. In some embodiments, the amino acid sequence of the hIgG1 Fc region comprises or consists of an alanine amino acid at position L234, an alanine amino acid at position L235, and a glycine amino acid at position P329, EU numbering according to Kabat; and comprises an amino acid sequence at least 85%, 86%, 87%, 88%, 89%, 90%, 91%, 92%, 93%, 94%, 95%, 96%, 97%, 98%, 99%, or 100% identical to the amino acid sequence of any one of SEQ ID NOS: 147-163.

### 5.3.2.2 Promotion of Heterodimerization

As described herein, in some embodiments, the fusion protein (or one or more polypeptide thereof) comprises a first and second Fc region. As described herein, in some embodiments, the heterologous moiety comprises a first and second Fc region. In some embodiments, the first Ig Fc region and the second Ig Fc region each comprise one or more amino acid modification relative to each other to promote heterodimerization. Ig (e.g., IgG) derived heterodimeric formats can be generated by methods known in the art, e.g., by forced heavy chain heterodimerization. Forced heavy chain heterodimerization can be obtained using known methods in the art, e.g., knob-in-hole or strand exchange engineered domains (SEED), see, e.g., Ji-Hee et al., “Immunoglobulin Fc Heterodimer Platform Technology: From Design to Applications in Therapeutic Antibodies and Proteins” Frontiers in Immunology, v7 (article 394) (2016) DOI=10.3389/fimmu.2016.00394 (hereinafter “Ji-Hee 2016”), the entire contents of which is incorporated herein by reference for all purposes.

In some embodiments, an interface of the first and the second Ig Fc regions is modified, e.g., introduction of an amino acid substitution, to increase heterodimerization, e.g., relative to a non-modified interface, e.g., a naturally occurring interface. For example, dimerization of the first and second Ig Fc regions can be enhanced by providing an Ig Fc interface of a first and a second Fc region with one or more of: a paired protuberance-cavity (“knob-in-hole”), an electrostatic interaction, or a strand-exchange, such that a greater ratio of heteromultimer to homomultimer forms, e.g., relative to a non-modified interface.

Knob-in-Hole amino acid pairing modifications are known in the art, and described in e.g., U.S. Pat. Nos. 5,731,116; 7,476,724; Ji-Hee 2016; and Ridgway, J. “'Knobs-into-holes’ engineering of antibody CH3 domains for heavy chain heterodimerization” et al. Prot. Engineering 9 (7): 617-621 (1996), the full contents of each of which is incorporated herein by reference. Generally, Knob-in-Hole comprises 1) introducing one or more amino acid substitutions in the CH3 domain of one or both of the first and second subject Ig Fc regions to promote heterodimerization; and 2) combining the modified Ig Fc regions under conditions that promote heterodimerization. “Knobs” are typically created by substituting a small amino acid in a parental Ig Fc region with a larger amino acid (e.g., T366Y or T366W); “holes” are created by substituting a larger residue in a parental Ig Fc region with a smaller amino acid (e.g., Y407T, T366S, 11368A, or Y407V). Exemplary Knob-in-Hole mutations include S354C, T366W in the “knob” Ig Fc region and Y349C, T366S, L368A, Y407V in the “hole” Ig Fc region. Other exemplary Knob-in-Hole mutations, which can be incorporated into any one or more of the embodiments, are provided in Table 14, with additional exemplary optional stabilizing Ig Fc cysteine mutations.

TABLE 14
Exemplary Knob-in-hole and Stabilizing Cysteine Modifications

| | | |
|---|---|---|
| Amino Acid Position | Knob Ig Fc | Hole Ig Fc |
| (EU numbering | Amino Acid | Amino Acid |
| according to Kabat) | Substitution | Substitution |

Knob-in Hole Amio Acid Substitutions

| | | |
|---|---|---|
| T366 | T366W | T366S |
| L368 | — | L368A |
| Y407 | — | Y407V |

Stabilizing Cysteine Amino Acid Substitutions

| | | |
|---|---|---|
| S354 | S354C | — |
| Y349 | — | Y349C |

The amino acid sequence of exemplary Fc regions that are known in the art to promote heterodimerization is provided in Table 15.

TABLE 15
The Amino Acid Sequence of Exemplary Pairs of Modified Heterodimeric Fc
Regions

| | | SEQ |
|---|---|---|
| Description | Amino Acid Sequence | ID NO |
| hIgG1 CH2 Region + | PCPAPELLGGPSVFLFPPKPKDTLMISRTPEVTCVVVDVSHED | 173 |
| CH3 Region | PEVKFNWYVDGVEVHNAKTKPREEQYNSTYRVVSVLTVLHQDW | |
| Knob T366W | LNGKEYKCKVSNKALPAPIEKTISKAKGQPREPQVYTLPPSRD | |
| With C-terminal Lysine | ELTKNQVSLWCLVKGFYPSDIAVEWESNGQPENNYKTTPPVLD | |
| | SDGSFFLYSKLTVDKSRWQQGNVFSCSVMHEALHNHYTQKSLS | |
| | LSPGK | |
| hIgG1 CH2 Region + | PCPAPELLGGPSVFLFPPKPKDTLMISRTPEVTCVVVDVSHED | 174 |
| CH3 Region | PEVKFNWYVDGVEVHNAKTKPREEQYNSTYRVVSVLTVLHQDW | |
| Knob T366W | LNGKEYKCKVSNKALPAPIEKTISKAKGQPREPQVYTLPPSRD | |
| Without C-terminal | ELTKNQVSLWCLVKGFYPSDIAVEWESNGQPENNYKTTPPVLD | |
| Lysine | SDGSFFLYSKLTVDKSRWQQGNVFSCSVMHEALHNHYTQKSLS | |
| | LSPG | |
| hIgG1 Partial Hinge | TCPPCPAPELLGGPSVFLFPPKPKDTLMISRTPEVTCVVVDVS | 175 |
| Region + CH2 Region + | HEDPEVKFNWYVDGVEVHNAKTKPREEQYNSTYRVVSVLTVLH | |
| CH3 Region | QDWLNGKEYKCKVSNKALPAPIEKTISKAKGQPREPQVYTLPP | |
| Knob T366W | SRDELTKNQVSLWCLVKGFYPSDIAVEWESNGQPENNYKTTPP | |
| With C-terminal Lysine | VLDSDGSFFLYSKLTVDKSRWQQGNVFSCSVMHEALHNHYTQK | |
| | SLSLSPGK | |
| hIgG1 Partial Hinge | TCPPCPAPELLGGPSVFLFPPKPKDTLMISRTPEVTCVVVDVS | 176 |
| Region + CH2 Region + | HEDPEVKFNWYVDGVEVHNAKTKPREEQYNSTYRVVSVLTVLH | |
| CH3 Region | QDWLNGKEYKCKVSNKALPAPIEKTISKAKGQPREPQVYTLPP | |
| Knob T366W | SRDELTKNQVSLWCLVKGFYPSDIAVEWESNGQPENNYKTTPP | |
| Without C-terminal | VLDSDGSFFLYSKLTVDKSRWQQGNVFSCSVMHEALHNHYTQK | |
| Lysine | SLSLSPG | |
| hIgG1 Hinge Region + | EPKSCDKTHTCPPCPAPELLGGPSVFLFPPKPKDTLMISRTPE | 177 |
| CH2 Region + CH3 | VTCVVVDVSHEDPEVKFNWYVDGVEVHNAKTKPREEQYNSTYR | |
| Region | VVSVLTVLHQDWLNGKEYKCKVSNKALPAPIEKTISKAKGQPR | |
| Knob T366W | EPQVYTLPPSRDELTKNQVSLWCLVKGFYPSDIAVEWESNGQP | |
| With C-terminal Lysine | ENNYKTTPPVLDSDGSFFLYSKLTVDKSRWQQGNVFSCSVMHE | |
| | ALHNHYTQKSLSLSPGK | |
| hIgG1 Hinge Region + | EPKSCDKTHTCPPCPAPELLGGPSVFLFPPKPKDTLMISRTPE | 178 |
| CH2 Region + CH3 | VTCVVVDVSHEDPEVKFNWYVDGVEVHNAKTKPREEQYNSTYR | |
| Region | VVSVLTVLHQDWLNGKEYKCKVSNKALPAPIEKTISKAKGQPR | |
| Knob T366W | EPQVYTLPPSRDELTKNQVSLWCLVKGFYPSDIAVEWESNGQP | |
| Without C-terminal | ENNYKTTPPVLDSDGSFFLYSKLTVDKSRWQQGNVFSCSVMHE | |
| Lysine | ALHNHYTQKSLSLSPG | |
| hIgG1 CH1+ Hinge | ASTKGPSVFPLAPSSKSTSGGTAALGCLVKDYFPEPVTVSWNS | 179 |
| Region + CH2 Region + | GALTSGVHTFPAVLQSSGLYSLSSVVTVPSSSLGTQTYICNVN | |
| CH3 Region | HKPSNTKVDKKVEPKSCDKTHTCPPCPAPELLGGPSVFLFPPK | |
| Knob T366W | PKDTLMISRTPEVTCVVVDVSHEDPEVKFNWYVDGVEVHNAKT | |
| With C-terminal Lysine | KPREEQYNSTYRVVSVLTVLHQDWLNGKEYKCKVSNKALPAPI | |
| | EKTISKAKGQPREPQVYTLPPCRDELTKNQVSLWCLVKGFYPS | |
| | DIAVEWESNGQPENNYKTTPPVLDSDGSFFLYSKLTVDKSRWQ | |
| | QGNVFSCSVMHEALHNHYTQKSLSLSPGK | |
| hIgG1 CH1 + Hinge | ASTKGPSVFPLAPSSKSTSGGTAALGCLVKDYFPEPVTVSWNS | 180 |
| Region + CH2 Region + | GALTSGVHTFPAVLQSSGLYSLSSVVTVPSSSLGTQTYICNVN | |
| CH3 Region | HKPSNTKVDKKVEPKSCDKTHTCPPCPAPELLGGPSVFLFPPK | |
| Knob T366W | PKDTLMISRTPEVTCVVVDVSHEDPEVKFNWYVDGVEVHNAKT | |
| Without C-terminal | KPREEQYNSTYRVVSVLTVLHQDWLNGKEYKCKVSNKALPAPI | |
| Lysine | EKTISKAKGQPREPQVYTLPPSRDELTKNQVSLWCLVKGFYPS | |
| | DIAVEWESNGQPENNYKTTPPVLDSDGSFFLYSKLTVDKSRWQ | |
| | QGNVFSCSVMHEALHNHYTQKSLSLSPG | |
| hIgG1 CH2 Region + | PCPAPELLGGPSVFLFPPKPKDTLMISRTPEVTCVVVDVSHED | 181 |
| CH3 Region | PEVKFNWYVDGVEVHNAKTKPREEQYNSTYRVVSVLTVLHQDW | |
| Knob T366W/S354C | LNGKEYKCKVSNKALPAPIEKTISKAKGQPREPQVYTLPPCRD | |
| With C-terminal Lysine | ELTKNQVSLWCLVKGFYPSDIAVEWESNGQPENNYKTTPPVLD | |
| | SDGSFFLYSKLTVDKSRWQQGNVFSCSVMHEALHNHYTQKSLS | |
| | LSPGK | |
| hIgG1 CH2 Region + | PCPAPELLGGPSVFLFPPKPKDTLMISRTPEVTCVVVDVSHED | 182 |
| CH3 Region | PEVKFNWYVDGVEVHNAKTKPREEQYNSTYRVVSVLTVLHQDW | |
| Knob T366W/S354C | LNGKEYKCKVSNKALPAPIEKTISKAKGQPREPQVYTLPPCRD | |
| Without C-terminal | ELTKNQVSLWCLVKGFYPSDIAVEWESNGQPENNYKTTPPVLD | |
| Lysine | SDGSFFLYSKLTVDKSRWQQGNVFSCSVMHEALHNHYTQKSLS | |
| | LSPG | |
| hIgG1 Partial Hinge | TCPPCPAPELLGGPSVFLFPPKPKDTLMISRTPEVTCVVVDVS | 183 |
| Region + CH2 Region + | HEDPEVKFNWYVDGVEVHNAKTKPREEQYNSTYRVVSVLTVLH | |
| CH3 Region | QDWLNGKEYKCKVSNKALPAPIEKTISKAKGQPREPQVYTLPP | |
| Knob T366W/S354C | CRDELTKNQVSLWCLVKGFYPSDIAVEWESNGQPENNYKTTPP | |
| With C-terminal Lysine | VLDSDGSFFLYSKLTVDKSRWQQGNVFSCSVMHEALHNHYTQK | |
| | SLSLSPGK | |
| hIgG1 Partial Hinge | TCPPCPAPELLGGPSVFLFPPKPKDTLMISRTPEVTCVVVDVS | 184 |
| Region + CH2 Region + | HEDPEVKFNWYVDGVEVHNAKTKPREEQYNSTYRVVSVLTVLH | |
| CH3 Region | QDWLNGKEYKCKVSNKALPAPIEKTISKAKGQPREPQVYTLPP | |
| Knob T366W/S354C | CRDELTKNQVSLWCLVKGFYPSDIAVEWESNGQPENNYKTTPP | |
| Without C-terminal | VLDSDGSFFLYSKLTVDKSRWQQGNVFSCSVMHEALHNHYTQK | |
| Lysine | SLSLSPG | |
| hIgG1 Hinge Region + | EPKSCDKTHTCPPCPAPELLGGPSVFLFPPKPKDTLMISRTPE | 185 |
| CH2 Region + CH3 | VTCVVVDVSHEDPEVKFNWYVDGVEVHNAKTKPREEQYNSTYR | |
| Region | VVSVLTVLHQDWLNGKEYKCKVSNKALPAPIEKTISKAKGQPR | |
| Knob T366W/S354C | EPQVYTLPPCRDELTKNQVSLWCLVKGFYPSDIAVEWESNGQP | |
| With C-terminal Lysine | ENNYKTTPPVLDSDGSFFLYSKLTVDKSRWQQGNVFSCSVMHE | |
| | ALHNHYTQKSLSLSPGK | |
| hIgG1 Hinge Region + | EPKSCDKTHTCPPCPAPELLGGPSVFLFPPKPKDTLMISRTPE | 186 |
| CH2 Region + CH3 | VTCVVVDVSHEDPEVKFNWYVDGVEVHNAKTKPREEQYNSTYR | |
| Region | VVSVLTVLHQDWLNGKEYKCKVSNKALPAPIEKTISKAKGQPR | |
| Knob T366W/S354C | EPQVYTLPPCRDELTKNQVSLWCLVKGFYPSDIAVEWESNGQP | |
| Without C-terminal | ENNYKTTPPVLDSDGSFFLYSKLTVDKSRWQQGNVESCSVMHE | |
| Lysine | ALHNHYTQKSLSLSPG | |
| hIgG1 CH1+ Hinge | ASTKGPSVFPLAPSSKSTSGGTAALGCLVKDYFPEPVTVSWNS | 187 |
| Region + CH2 Region + | GALTSGVHTFPAVLQSSGLYSLSSVVTVPSSSLGTQTYICNVN | |
| CH3 Region | HKPSNTKVDKKVEPKSCDKTHTCPPCPAPELLGGPSVFLFPPK | |
| Knob T366W/S354C | PKDTLMISRTPEVTCVVVDVSHEDPEVKFNWYVDGVEVHNAKT | |
| With C-terminal Lysine | KPREEQYNSTYRVVSVLTVLHQDWLNGKEYKCKVSNKALPAPI | |
| | EKTISKAKGQPREPQVYTLPPCRDELTKNQVSLWCLVKGFYPS | |
| | DIAVEWESNGQPENNYKTTPPVLDSDGSFFLYSKLTVDKSRWQ | |
| | QGNVFSCSVMHEALHNHYTQKSLSLSPGK | |
| hIgG1 CH1 + Hinge | ASTKGPSVFPLAPSSKSTSGGTAALGCLVKDYFPEPVTVSWNS | 188 |
| Region + CH2 Region + | GALTSGVHTFPAVLQSSGLYSLSSVVTVPSSSLGTQTYICNVN | |
| CH3 Region | HKPSNTKVDKKVEPKSCDKTHTCPPCPAPELLGGPSVFLFPPK | |
| Knob T366W/S354C | PKDTLMISRTPEVTCVVVDVSHEDPEVKFNWYVDGVEVHNAKT | |
| Without C-terminal | KPREEQYNSTYRVVSVLTVLHQDWLNGKEYKCKVSNKALPAPI | |
| Lysine | EKTISKAKGQPREPQVYTLPPCRDELTKNQVSLWCLVKGFYPS | |
| | DIAVEWESNGQPENNYKTTPPVLDSDGSFFLYSKLTVDKSRWQ | |
| | QGNVFSCSVMHEALHNHYTQKSLSLSPG | |
| hIgG1 CH2 Region + | PCPAPELLGGPSVFLFPPKPKDTLMISRTPEVTCVVVDVSHED | 189 |
| CH3 Region | PEVKFNWYVDGVEVHNAKTKPREEQYNSTYRVVSVLTVLHQDW | |
| Hole | LNGKEYKCKVSNKALPAPIEKTISKAKGQPREPQVYTLPPSRD | |
| T366S/L368A/Y407V | ELTKNQVSLSCAVKGFYPSDIAVEWESNGQPENNYKTTPPVLD | |
| With C-terminal Lysine | SDGSFFLVSKLTVDKSRWQQGNVFSCSVMHEALHNHYTQKSLS | |
| | LSPGK | |
| hIgG1 CH2 Region + | PCPAPELLGGPSVFLFPPKPKDTLMISRTPEVTCVVVDVSHED | 190 |
| CH3 Region | PEVKFNWYVDGVEVHNAKTKPREEQYNSTYRVVSVLTVLHQDW | |
| Hole | LNGKEYKCKVSNKALPAPIEKTISKAKGQPREPQVYTLPPSRD | |
| T366S/L368A/Y407V | ELTKNQVSLSCAVKGFYPSDIAVEWESNGQPENNYKTTPPVLD | |
| Without C-terminal | SDGSFFLVSKLTVDKSRWQQGNVFSCSVMHEALHNHYTQKSLS | |
| Lysine | LSPG | |
| hIgG1 Partial Hinge | TCPPCPAPELLGGPSVFLFPPKPKDTLMISRTPEVTCVVVDVS | 191 |
| Region + CH2 Region + | HEDPEVKFNWYVDGVEVHNAKTKPREEQYNSTYRVVSVLTVLH | |
| CH3 Region | QDWLNGKEYKCKVSNKALPAPIEKTISKAKGQPREPQVYTLPP | |
| Hole | SRDELTKNQVSLSCAVKGFYPSDIAVEWESNGQPENNYKTTPP | |
| T366S/L368A/Y407V | VLDSDGSFFLVSKLTVDKSRWQQGNVFSCSVMHEALHNHYTQK | |
| With C-terminal Lysine | SLSLSPGK | |
| hIgG1 Partial Hinge | TCPPCPAPELLGGPSVFLFPPKPKDTLMISRTPEVTCVVVDVS | 192 |
| Region + CH2 Region + | HEDPEVKFNWYVDGVEVHNAKTKPREEQYNSTYRVVSVLTVLH | |
| CH3 Region | QDWLNGKEYKCKVSNKALPAPIEKTISKAKGQPREPQVYTLPP | |
| Hole | SRDELTKNQVSLSCAVKGFYPSDIAVEWESNGQPENNYKTTPP | |
| T366S/L368A/Y407V | VLDSDGSFFLVSKLTVDKSRWQQGNVFSCSVMHEALHNHYTQK | |
| Without C-terminal | SLSLSPG | |
| Lysine | | |
| hIgG1 Hinge Region + | EPKSCDKTHTCPPCPAPELLGGPSVFLFPPKPKDTLMISRTPE | 193 |
| CH2 Region + CH3 | VTCVVVDVSHEDPEVKFNWYVDGVEVHNAKTKPREEQYNSTYR | |
| Region | VVSVLTVLHQDWLNGKEYKCKVSNKALPAPIEKTISKAKGQPR | |
| Hole | EPQVYTLPPSRDELTKNQVSLSCAVKGFYPSDIAVEWESNGQP | |
| T366S/L368A/Y407V | ENNYKTTPPVLDSDGSFFLVSKLTVDKSRWQQGNVESCSVMHE | |
| With C-terminal Lysine | ALHNHYTQKSLSLSPGK | |
| hIgG1 Hinge Region + | EPKSCDKTHTCPPCPAPELLGGPSVFLFPPKPKDTLMISRTPE | 194 |
| CH2 Region + CH3 | VTCVVVDVSHEDPEVKFNWYVDGVEVHNAKTKPREEQYNSTYR | |
| Region | VVSVLTVLHQDWLNGKEYKCKVSNKALPAPIEKTISKAKGQPR | |
| Hole | EPQVYTLPPSRDELTKNQVSLSCAVKGFYPSDIAVEWESNGQP | |
| T366S/L368A/Y407V | ENNYKTTPPVLDSDGSFFLVSKLTVDKSRWQQGNVFSCSVMHE | |
| Without C-terminal | ALHNHYTQKSLSLSPG | |
| Lysine | | |
| hIgG1 CH1+ Hinge | ASTKGPSVFPLAPSSKSTSGGTAALGCLVKDYFPEPVTVSWNS | 195 |
| Region + CH2 Region + | GALTSGVHTFPAVLQSSGLYSLSSVVTVPSSSLGTQTYICNVN | |
| CH3 Region | HKPSNTKVDKKVEPKSCDKTHTCPPCPAPELLGGPSVFLFPPK | |
| Hole | PKDTLMISRTPEVTCVVVDVSHEDPEVKFNWYVDGVEVHNAKT | |
| T366S/L368A/Y407V | KPREEQYNSTYRVVSVLTVLHQDWLNGKEYKCKVSNKALPAPI | |
| With C-terminal Lysine | EKTISKAKGQPREPQVYTLPPSRDELTKNQVSLSCAVKGFYPS | |
| | DIAVEWESNGQPENNYKTTPPVLDSDGSFFLVSKLTVDKSRWQ | |
| | QGNVFSCSVMHEALHNHYTQKSLSLSPGK | |
| hIgG1 CH1 + Hinge | ASTKGPSVFPLAPSSKSTSGGTAALGCLVKDYFPEPVTVSWNS | 196 |
| Region + CH2 Region + | GALTSGVHTFPAVLQSSGLYSLSSVVTVPSSSLGTQTYICNVN | |
| CH3 Region | HKPSNTKVDKKVEPKSCDKTHTCPPCPAPELLGGPSVFLFPPK | |
| Hole | PKDTLMISRTPEVTCVVVDVSHEDPEVKFNWYVDGVEVHNAKT | |
| T366S/L368A/Y407V | KPREEQYNSTYRVVSVLTVLHQDWLNGKEYKCKVSNKALPAPI | |
| Without C-terminal | EKTISKAKGQPREPQVYTLPPSRDELTKNQVSLSCAVKGFYPS | |
| Lysine | DIAVEWESNGQPENNYKTTPPVLDSDGSFFLVSKLTVDKSRWQ | |
| | QGNVFSCSVMHEALHNHYTQKSLSLSPG | |
| hIgG1 CH2 Region + | PCPAPELLGGPSVFLFPPKPKDTLMISRTPEVTCVVVDVSHED | 197 |
| CH3 Region | PEVKFNWYVDGVEVHNAKTKPREEQYNSTYRVVSVLTVLHQDW | |
| Hole | LNGKEYKCKVSNKALPAPIEKTISKAKGQPREPQVYTLPPSRD | |
| T366S/L368A/Y407V/ | ELTKNQVSLSCAVKGFYPSDIAVEWESNGQPENNYKTTPPVLD | |
| Y349C | SDGSFFLVSKLTVDKSRWQQGNVFSCSVMHEALHNHYTQKSLS | |
| With C-terminal Lysine | LSPGK | |
| hIgG1 CH2 Region + | PCPAPELLGGPSVFLFPPKPKDTLMISRTPEVTCVVVDVSHED | 198 |
| CH3 Region | PEVKFNWYVDGVEVHNAKTKPREEQYNSTYRVVSVLTVLHQDW | |
| Hole | LNGKEYKCKVSNKALPAPIEKTISKAKGQPREPQVYTLPPSRD | |
| T366S/L368A/Y407V/ | ELTKNQVSLSCAVKGFYPSDIAVEWESNGQPENNYKTTPPVLD | |
| Y349C | SDGSFFLVSKLTVDKSRWQQGNVFSCSVMHEALHNHYTQKSLS | |
| Without C-terminal | LSPG | |
| Lysine | | |
| hIgG1 Partial Hinge | TCPPCPAPELLGGPSVFLFPPKPKDTLMISRTPEVTCVVVDVS | 199 |
| Region + CH2 Region + | HEDPEVKFNWYVDGVEVHNAKTKPREEQYNSTYRVVSVLTVLH | |
| CH3 Region | QDWLNGKEYKCKVSNKALPAPIEKTISKAKGQPREPQVCTLPP | |
| Hole | SRDELTKNQVSLSCAVKGFYPSDIAVEWESNGQPENNYKTTPP | |
| T366S/L368A/Y407V/ | VLDSDGSFFLVSKLTVDKSRWQQGNVFSCSVMHEALHNHYTQK | |
| Y349C | SLSLSPGK | |
| With C-terminal Lysine | | |
| hIgG1 Partial Hinge | TCPPCPAPELLGGPSVFLFPPKPKDTLMISRTPEVTCVVVDVS | 200 |
| Region + CH2 Region + | HEDPEVKFNWYVDGVEVHNAKTKPREEQYNSTYRVVSVLTVLH | |
| CH3 Region | QDWLNGKEYKCKVSNKALPAPIEKTISKAKGQPREPQVCTLPP | |
| Hole | SRDELTKNQVSLSCAVKGFYPSDIAVEWESNGQPENNYKTTPP | |
| T366S/L368A/Y407V/ | VLDSDGSFFLVSKLTVDKSRWQQGNVFSCSVMHEALHNHYTQK | |
| Y349C | SLSLSPG | |
| Without C-terminal | | |
| Lysine | | |
| hIgG1 Hinge Region + | EPKSCDKTHTCPPCPAPELLGGPSVFLFPPKPKDTLMISRTPE | 201 |
| CH2 Region + CH3 | VTCVVVDVSHEDPEVKFNWYVDGVEVHNAKTKPREEQYNSTYR | |
| Region | VVSVLTVLHQDWLNGKEYKCKVSNKALPAPIEKTISKAKGQPR | |
| Hole | EPQVCTLPPSRDELTKNQVSLSCAVKGFYPSDIAVEWESNGQP | |
| T366S/L368A/Y407V/ | ENNYKTTPPVLDSDGSFFLVSKLTVDKSRWQQGNVFSCSVMHE | |
| Y349C | ALHNHYTQKSLSLSPGK | |
| With C-terminal Lysine | | |
| hIgG1 Hinge Region + | EPKSCDKTHTCPPCPAPELLGGPSVFLFPPKPKDTLMISRTPE | 202 |
| CH2 Region + CH3 | VTCVVVDVSHEDPEVKFNWYVDGVEVHNAKTKPREEQYNSTYR | |
| Region | VVSVLTVLHQDWLNGKEYKCKVSNKALPAPIEKTISKAKGQPR | |
| Hole | EPQVCTLPPSRDELTKNQVSLSCAVKGFYPSDIAVEWESNGQP | |
| T366S/L368A/Y407V/ | ENNYKTTPPVLDSDGSFFLVSKLTVDKSRWQQGNVFSCSVMHE | |
| Y349C | ALHNHYTQKSLSLSPG | |
| Without C-terminal | | |
| Lysine | | |
| hIgG1 CH1+ Hinge | ASTKGPSVFPLAPSSKSTSGGTAALGCLVKDYFPEPVTVSWNS | 203 |
| Region + CH2 Region + | GALTSGVHTFPAVLQSSGLYSLSSVVTVPSSSLGTQTYICNVN | |
| CH3 Region | HKPSNTKVDKKVEPKSCDKTHTCPPCPAPELLGGPSVFLFPPK | |
| Hole | PKDTLMISRTPEVTCVVVDVSHEDPEVKFNWYVDGVEVHNAKT | |
| T366S/L368A/Y407V/ | KPREEQYNSTYRVVSVLTVLHQDWLNGKEYKCKVSNKALPAPI | |
| Y349C | EKTISKAKGQPREPQVCTLPPSRDELTKNQVSLSCAVKGFYPS | |
| With C-terminal Lysine | DIAVEWESNGQPENNYKTTPPVLDSDGSFFLVSKLTVDKSRWQ | |
| | QGNVFSCSVMHEALHNHYTQKSLSLSPGK | |
| hIgG1 CH1 + Hinge | ASTKGPSVFPLAPSSKSTSGGTAALGCLVKDYFPEPVTVSWNS | 204 |
| Region + CH2 Region + | GALTSGVHTFPAVLQSSGLYSLSSVVTVPSSSLGTQTYICNVN | |
| CH3 Region | HKPSNTKVDKKVEPKSCDKTHTCPPCPAPELLGGPSVFLFPPK | |
| Hole | PKDTLMISRTPEVTCVVVDVSHEDPEVKFNWYVDGVEVHNAKT | |
| T366S/L368A/Y407V/ | KPREEQYNSTYRVVSVLTVLHQDWLNGKEYKCKVSNKALPAPI | |
| Y349C | EKTISKAKGQPREPQVCTLPPSRDELTKNQVSLSCAVKGFYPS | |
| Without C-terminal | DIAVEWESNGQPENNYKTTPPVLDSDGSFFLVSKLTVDKSRWQ | |
| Lysine | QGNVFSCSVMHEALHNHYTQKSLSLSPG | |

As described herein, in some embodiments, the fusion protein (or one or more polypeptide thereof) comprises a first Fc region and a second Pc region.

In some embodiments, the amino acid sequence of the first Fc region comprises a T366W amino acid substitution, EU numbering according to Kabat; and the second the amino acid sequence of the Fc region comprises each of the following amino acid substitutions: T366S, L368A, and Y407V, EU numbering according to Kabat; each relative to the amino acid sequence of an exemplary reference Fc region (e.g., a reference Fc region set forth in Table 12). In some embodiments, the amino acid sequence of the first h further comprises a S354C amino acid substitution, EU numbering according to Kabat; and the amino acid sequence of the second Fc region comprises a Y349C amino acid substitution, EU numbering according to Kabat; each relative to the amino acid sequence of an exemplary reference Fc region (e.g., a reference Fc region set forth in Table 12).

In some embodiments, the amino acid sequence of the first Fc region comprises each of the following amino acid substitutions: T366W and a S354C, EU numbering according to Kabat; and the second the amino acid sequence of the Fc region comprises each of the following amino acid substitutions: T366S, L368A, Y407V, and Y349C, EU numbering according to Kabat; each relative to the amino acid sequence of an exemplary reference Fc region (e.g., a reference Fc region set forth in Table 12).

In some embodiments, the amino acid sequence of the second Fc region comprises a T366W amino acid substitution, EU numbering according to Kabat; and the second the amino acid sequence of the Fc region comprises each of the following amino acid substitutions: T366S, L368A, and Y407V, EU numbering according to Kabat; each relative to the amino acid sequence of an exemplary reference Fc region (e.g., a reference Fc region set forth in Table 12). In some embodiments, the amino acid sequence of the second h further comprises a S354C amino acid substitution, EU numbering according to Kabat; and the amino acid sequence of the second Fc region comprises a Y349C amino acid substitution, EU numbering according to Kabat; each relative to the amino acid sequence of an exemplary reference Fc region (e.g., a reference Fc region set forth in Table 12).

In some embodiments, the amino acid sequence of the second Fc region comprises each of the following amino acid substitutions: T366W and a S354C, EU numbering according to Kabat; and the second the amino acid sequence of the Fc region comprises each of the following amino acid substitutions: T366S, L368A, Y407V, and Y349C, EU numbering according to Kabat; each relative to the amino acid sequence of an exemplary reference Fc region (e.g., a reference Fc region set forth in Table 12).

In some embodiments, the amino acid sequence of the first Fc region comprises a tryptophan amino acid at position T366, EU numbering according to Kabat; and the amino acid sequence of the second Fc region comprises a serine amino acid at position T366, an alanine amino acid at position L368, and a valine amino acid at position Y407, EU numbering according to Kabat.

In some embodiments, the amino acid sequence of the first Fc region comprises a tryptophan amino acid at position T366 and a cysteine amino acid at position S354, EU numbering according to Kabat; and the amino acid sequence of the second Fc region comprises a serine amino acid at position T366, an alanine amino acid at position L368, a valine amino acid at position Y407, and a cysteine amino acid at position Y349, EU numbering according to Kabat.

In some embodiments, the amino acid sequence of the first Fc region comprises a tryptophan amino acid at position T366, EU numbering according to Kabat, and is at least 85%, 86%, 87%, 88%, 89%, 90%, 91%, 92%, 93%, 94%, 95%, 96%, 97%, 98%, 99%, or 100% identical to the amino acid sequence of any one of SEQ ID NOS: 173-180; and the amino acid sequence of the second Fc region comprises a serine amino acid at position T366, an alanine amino acid at position L368, and a valine amino acid at position Y407, EU numbering according to Kabat, and is at least 85%, 86%, 87%, 88%, 89%, 90%, 91%, 92%, 93%, 94%, 95%, 96%, 97%, 98%, 99%, or 100% identical to the amino acid sequence of any one of SEQ ID NOS: 189-196.

In some embodiments, the amino acid sequence of the first Fc region comprises a tryptophan amino acid at position T366 and a cysteine amino acid at position S354, EU numbering according to Kabat, and is at least 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 182-188; and the amino acid sequence of the second Fc region comprises a serine amino acid at position T366, an alanine amino acid at position L368, a valine amino acid at position Y407, and a cysteine amino acid at position Y349, EU numbering according to Kabat, and is at least 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 197-204.

In some embodiments, the amino acid sequence of the first Fc region comprises a tryptophan amino acid at position T366, EU numbering according to Kabat, and is at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 173; and the amino acid sequence of the second Fc region comprises a serine amino acid at position T366, an alanine amino acid at position

L368, and a valine amino acid at position Y407, EU numbering according to Kabat, and is at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 189.

In some embodiments, the amino acid sequence of the first Fc region comprises a tryptophan amino acid at position T366, EU numbering according to Kabat, and is at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 174; and the amino acid sequence of the second Fc region comprises a serine amino acid at position T366, an alanine amino acid at position L368, and a valine amino acid at position Y407, EU numbering according to Kabat, and is at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 190.

In some embodiments, the amino acid sequence of the first Fc region comprises a tryptophan amino acid at position T366, EU numbering according to Kabat, and is at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 175; and the amino acid sequence of the second Fc region comprises a serine amino acid at position T366, an alanine amino acid at position L368, and a valine amino acid at position Y407, EU numbering according to Kabat, and is at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 191.

In some embodiments, the amino acid sequence of the first Fc region comprises a W amino acid at position T366, EU numbering according to Kabat, and is at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 176; and the amino acid sequence of the second Fc region comprises a serine amino acid at position T366, an alanine amino acid at position L368, and a valine amino acid at position Y407, EU numbering according to Kabat, and is at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 192.

In some embodiments, the amino acid sequence of the first Fc region comprises a tryptophan amino acid at position T366, EU numbering according to Kabat, and is at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 177; and the amino acid sequence of the second Fc region comprises a serine amino acid at position T366, an alanine amino acid at position L368, and a valine amino acid at position Y407, EU numbering according to Kabat, and is at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 193.

In some embodiments, the amino acid sequence of the first Fc region comprises a tryptophan amino acid at position T366, EU numbering according to Kabat, and is at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 178; and the amino acid sequence of the second Fc region comprises a serine amino acid at position T366, an alanine amino acid at position L368, and a valine amino acid at position Y407, EU numbering according to Kabat, and is at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 194.

In some embodiments, the amino acid sequence of the first Fc region comprises a tryptophan amino acid at position T366, EU numbering according to Kabat, and is at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 179; and the amino acid sequence of the second Fc region comprises a serine amino acid at position T366, an alanine amino acid at position L368, and a valine amino acid at position Y407, EU numbering according to Kabat, and is at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 195.

In some embodiments, the amino acid sequence of the first Fc region comprises a tryptophan amino acid at position T366, EU numbering according to Kabat, and is at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 180; and the amino acid sequence of the second Fc region comprises a serine amino acid at position T366, an alanine amino acid at position L368, and a valine amino acid at position Y407, EU numbering according to Kabat, and is at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 196.

In some embodiments, the amino acid sequence of the first Fc region comprises a tryptophan amino acid at position T366 and a cysteine amino acid at position S354, EU numbering according to Kabat, and is at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 181; and the amino acid sequence of the second Fc region comprises a serine amino acid at position T366, an alanine amino acid at position L368, a valine amino acid at position Y407, and a cysteine amino acid at position Y349, EU numbering according to Kabat, and is at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 197.

In some embodiments, the amino acid sequence of the first Fc region comprises a tryptophan amino acid at position T366 and a cysteine amino acid at position S354, EU numbering according to Kabat, and is at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 182; and the amino acid sequence of the second Fc region comprises a serine amino acid at position T366, an alanine amino acid at position L368, a valine amino acid at position Y407, and a cysteine amino acid at position Y349, EU numbering according to Kabat, and is at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 198.

In some embodiments, the amino acid sequence of the first Fc region comprises a tryptophan amino acid at position T366 and a cysteine amino acid at position S354, EU numbering according to Kabat, and is at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 183; and the amino acid sequence of the second Fc region comprises a serine amino acid at position T366, an alanine amino acid at position L368, a valine amino acid at position Y407, and a cysteine amino acid at position Y349, EU numbering according to Kabat, and is at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 199.

In some embodiments, the amino acid sequence of the first Fc region comprises a tryptophan amino acid at position T366 and a cysteine amino acid at position S354, EU numbering according to Kabat, and is at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 184; and the amino acid sequence of the second Fc region comprises a serine amino acid at position T366, an alanine amino acid at position L368, a valine amino acid at position Y407, and a cysteine amino acid at position Y349, EU numbering according to Kabat, and is at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 200.

In some embodiments, the amino acid sequence of the first Fc region comprises a tryptophan amino acid at position T366 and a cysteine amino acid at position S354, EU numbering according to Kabat, and is at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 185; and the amino acid sequence of the second Fc region comprises a serine amino acid at position T366, an alanine amino acid at position L368, a valine amino acid at position Y407, and a cysteine amino acid at position Y349, EU numbering according to Kabat, and is at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 201.

In some embodiments, the amino acid sequence of the first Fc region comprises a tryptophan amino acid at position T366 and a cysteine amino acid at position S354, EU numbering according to Kabat, and is at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 186; and the amino acid sequence of the second Fc region comprises a serine amino acid at position T366, an alanine amino acid at position L368, a valine amino acid at position Y407, and a cysteine amino acid at position Y349, EU numbering according to Kabat, and is at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 202.

In some embodiments, the amino acid sequence of the first Fc region comprises a tryptophan amino acid at position T366 and a cysteine amino acid at position S354, EU numbering according to Kabat, and is at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 187; and the amino acid sequence of the second Fc region comprises a serine amino acid at position T366, an alanine amino acid at position L368, a valine amino acid at position Y407, and a cysteine amino acid at position Y349, EU numbering according to Kabat, and is at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 203.

In some embodiments, the amino acid sequence of the first Fc region comprises a tryptophan amino acid at position T366 and a cysteine amino acid at position S354, EU numbering according to Kabat, and is at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 188; and the amino acid sequence of the second Fc region comprises a serine amino acid at position T366, an alanine amino acid at position L368, a valine amino acid at position Y407, and a cysteine amino acid at position Y349, EU numbering according to Kabat, and is at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 204.

### 5.3.2.3 Exemplary Modified Fc Regions

As described herein, in some embodiments, the fusion protein (or one or more polypeptide thereof) comprises a first and second Fc region. In some embodiments, the first Fc region and the second Fc region each comprise multiple amino acid modifications described herein, e.g., one or more amino acid modification that decreases or abolishes one or more Fc effector function (e.g., antibody dependent cellular cytotoxicity (ADCC), antibody dependent cellular phagocytosis (ADCP), complement dependent cytotoxicity (CDC), and binding affinity to one or more human Fc receptor (e.g., an Fcγ receptor (e.g., FcγRI, FcγRIIa, FcγRIIc, FcγRIIIa, and/or FcγRIIIb (e.g., FcγRI, FcγIIa, and/or FcγIIIa))) (see, e.g., § 5.3.2.1); and one or more amino acid modification that promote heterodimerization of the first and second Fc regions (see, e.g., § 5.3.2.2).

In some embodiments, the first and second Fc region each comprise one or more amino acid modification that decreases or abolishes one or more Fc effector functions (e.g., antibody dependent cellular cytotoxicity (ADCC), antibody dependent cellular phagocytosis (ADCP), complement dependent cytotoxicity (CDC), and binding affinity to one or more human Fc receptor (e.g., an Fcγ receptor (e.g., FcγRI, FcγRIIa, FcγRIIc, FcγRIIIa, and/or FcγRIIIb (e.g., FcγRI, FcγIIa, and/or FcγIIIa))) (see, e.g., § 5.3.2.1); and one or more amino acid modification that promote heterodimerization of the first and second Fc regions (see, e.g., § 5.3.2.2).

The amino acid sequence of exemplary modified Fc regions is provided in Table 16.

TABLE 16
The Amino Acid Sequence of Exemplary Modified Fc Regions

| | | SEQ |
|---|---|---|
| Description | Amino Acid Sequence | ID NO |
| hIgG1 CH2 Region + | PCPAPEAAGGPSVFLFPPKPKDTLMISRTPEVTCVVVDVSHED | 205 |
| CH3 Region | PEVKFNWYVDGVEVHNAKTKPREEQYNSTYRVVSVLTVLHQDW | |
| Knob T366W/S354C | LNGKEYKCKVSNKALAAPIEKTISKAKGQPREPQVYTLPPCRD | |
| L234A/L235A/P329A | ELTKNQVSLWCLVKGFYPSDIAVEWESNGQPENNYKTTPPVLD | |
| With C-terminal Lysine | SDGSFFLYSKLTVDKSRWQQGNVFSCSVMHEALHNHYTQKSLS | |
| | LSPGK | |
| hIgG1 CH2 Region + | PCPAPEAAGGPSVFLFPPKPKDTLMISRTPEVTCVVVDVSHED | 206 |
| CH3 Region | PEVKFNWYVDGVEVHNAKTKPREEQYNSTYRVVSVLTVLHQDW | |
| Knob T366W/S354C | LNGKEYKCKVSNKALAAPIEKTISKAKGQPREPQVYTLPPCRD | |
| L234A/L235A/P329A | ELTKNQVSLWCLVKGFYPSDIAVEWESNGQPENNYKTTPPVLD | |
| Without C-terminal | SDGSFFLYSKLTVDKSRWQQGNVFSCSVMHEALHNHYTQKSLS | |
| Lysine | LSPG | |
| hIgG1 Partial Hinge | TCPPCPAPEAAGGPSVFLFPPKPKDTLMISRTPEVTCVVVDVS | 207 |
| Region + CH2 Region + | HEDPEVKFNWYVDGVEVHNAKTKPREEQYNSTYRVVSVLTVLH | |
| CH3 Region | QDWLNGKEYKCKVSNKALAAPIEKTISKAKGQPREPQVYTLPP | |
| Knob T366W/S354C | CRDELTKNQVSLWCLVKGFYPSDIAVEWESNGQPENNYKTTPP | |
| L234A/L235A/P329A | VLDSDGSFFLYSKLTVDKSRWQQGNVFSCSVMHEALHNHYTQK | |
| With C-terminal Lysine | SLSLSPGK | |
| hIgG1 Partial Hinge | TCPPCPAPEAAGGPSVFLFPPKPKDTLMISRTPEVTCVVVDVS | 208 |
| Region + CH2 Region + | HEDPEVKFNWYVDGVEVHNAKTKPREEQYNSTYRVVSVLTVLH | |
| CH3 Region | QDWLNGKEYKCKVSNKALAAPIEKTISKAKGQPREPQVYTLPP | |
| Knob T366W/S354C | CRDELTKNQVSLWCLVKGFYPSDIAVEWESNGQPENNYKTTPP | |
| L234A/L235A/P329A | VLDSDGSFFLYSKLTVDKSRWQQGNVFSCSVMHEALHNHYTQK | |
| Without C-terminal | SLSLSPG | |
| Lysine | | |
| hIgG1 Hinge Region + | EPKSCDKTHTCPPCPAPEAAGGPSVFLFPPKPKDTLMISRTPE | 209 |
| CH2 Region + CH3 | VTCVVVDVSHEDPEVKFNWYVDGVEVHNAKTKPREEQYNSTYR | |
| Region | VVSVLTVLHQDWLNGKEYKCKVSNKALAAPIEKTISKAKGQPR | |
| Knob T366W/S354C | EPQVYTLPPCRDELTKNQVSLWCLVKGFYPSDIAVEWESNGQP | |
| L234A/L235A/P329A | ENNYKTTPPVLDSDGSFFLYSKLTVDKSRWQQGNVFSCSVMHE | |
| With C-terminal Lysine | ALHNHYTQKSLSLSPGK | |
| hIgG1 Hinge Region + | EPKSCDKTHTCPPCPAPEAAGGPSVFLFPPKPKDTLMISRTPE | 210 |
| CH2 Region + CH3 | VTCVVVDVSHEDPEVKFNWYVDGVEVHNAKTKPREEQYNSTYR | |
| Region | VVSVLTVLHQDWLNGKEYKCKVSNKALAAPIEKTISKAKGQPR | |
| Knob T366W/S354C | EPQVYTLPPCRDELTKNQVSLWCLVKGFYPSDIAVEWESNGQP | |
| L234A/L235A/P329A | ENNYKTTPPVLDSDGSFFLYSKLTVDKSRWQQGNVFSCSVMHE | |
| Without C-terminal | ALHNHYTQKSLSLSPG | |
| Lysine | | |
| hIgG1 CH1+ Hinge | ASTKGPSVFPLAPSSKSTSGGTAALGCLVKDYFPEPVTVSWNS | 211 |
| Region + CH2 Region + | GALTSGVHTFPAVLQSSGLYSLSSVVTVPSSSLGTQTYICNVN | |
| CH3 Region | HKPSNTKVDKKVEPKSCDKTHTCPPCPAPEAAGGPSVFLFPPK | |
| Knob T366W/S354C | PKDTLMISRTPEVTCVVVDVSHEDPEVKFNWYVDGVEVHNAKT | |
| L234A/L235A/P329A | KPREEQYNSTYRVVSVLTVLHQDWLNGKEYKCKVSNKALAAPI | |
| With C-terminal Lysine | EKTISKAKGQPREPQVYTLPPCRDELTKNQVSLWCLVKGFYPS | |
| | DIAVEWESNGQPENNYKTTPPVLDSDGSFFLYSKLTVDKSRWQ | |
| | QGNVFSCSVMHEALHNHYTQKSLSLSPGK | |
| hIgG1 CH1 + Hinge | ASTKGPSVFPLAPSSKSTSGGTAALGCLVKDYFPEPVTVSWNS | 212 |
| Region + CH2 Region + | GALTSGVHTFPAVLQSSGLYSLSSVVTVPSSSLGTQTYICNVN | |
| CH3 Region | HKPSNTKVDKKVEPKSCDKTHTCPPCPAPEAAGGPSVFLFPPK | |
| Knob T366W/S354C | PKDTLMISRTPEVTCVVVDVSHEDPEVKFNWYVDGVEVHNAKT | |
| L234A/L235A/P329A | KPREEQYNSTYRVVSVLTVLHQDWLNGKEYKCKVSNKALAAPI | |
| Without C-terminal | EKTISKAKGQPREPQVYTLPPCRDELTKNQVSLWCLVKGFYPS | |
| Lysine | DIAVEWESNGQPENNYKTTPPVLDSDGSFFLYSKLTVDKSRWQ | |
| | QGNVFSCSVMHEALHNHYTQKSLSLSPG | |
| hIgG1 CH2 Region + | PCPAPEAAGGPSVFLFPPKPKDTLMISRTPEVTCVVVDVSHED | 213 |
| CH3 Region | PEVKFNWYVDGVEVHNAKTKPREEQYNSTYRVVSVLTVLHQDW | |
| Hole | LNGKEYKCKVSNKALAAPIEKTISKAKGQPREPQVYTLPPSRD | |
| T366S/L368A/Y407V/Y | ELTKNQVSLSCAVKGFYPSDIAVEWESNGQPENNYKTTPPVLD | |
| 349C | SDGSFFLVSKLTVDKSRWQQGNVFSCSVMHEALHNHYTQKSLS | |
| L234A/L235A/P329A | LSPGK | |
| With C-terminal Lysine | | |
| hIgG1 CH2 Region + | PCPAPEAAGGPSVFLFPPKPKDTLMISRTPEVTCVVVDVSHED | 214 |
| CH3 Region | PEVKFNWYVDGVEVHNAKTKPREEQYNSTYRVVSVLTVLHQDW | |
| Hole | LNGKEYKCKVSNKALAAPIEKTISKAKGQPREPQVYTLPPSRD | |
| T366S/L368A/Y407V/ | ELTKNQVSLSCAVKGFYPSDIAVEWESNGQPENNYKTTPPVLD | |
| Y349C | SDGSFFLVSKLTVDKSRWQQGNVFSCSVMHEALHNHYTQKSLS | |
| L234A/L235A/P329A | LSPG | |
| Without C-terminal | | |
| Lysine | | |
| hIgG1 Partial Hinge | TCPPCPAPEAAGGPSVFLFPPKPKDTLMISRTPEVTCVVVDVS | 215 |
| Region + CH2 Region + | HEDPEVKFNWYVDGVEVHNAKTKPREEQYNSTYRVVSVLTVLH | |
| CH3 Region | QDWLNGKEYKCKVSNKALAAPIEKTISKAKGQPREPQVCTLPP | |
| Hole | SRDELTKNQVSLSCAVKGFYPSDIAVEWESNGQPENNYKTTPP | |
| T366S/L368A/Y407V/ | VLDSDGSFFLVSKLTVDKSRWQQGNVFSCSVMHEALHNHYTQK | |
| Y349C | SLSLSPGK | |
| L234A/L235A/P329A | | |
| With C-terminal Lysine | | |
| hIgG1 Partial Hinge | TCPPCPAPEAAGGPSVFLFPPKPKDTLMISRTPEVTCVVVDVS | 216 |
| Region + CH2 Region + | HEDPEVKFNWYVDGVEVHNAKTKPREEQYNSTYRVVSVLTVLH | |
| CH3 Region | QDWLNGKEYKCKVSNKALAAPIEKTISKAKGQPREPQVCTLPP | |
| Hole | SRDELTKNQVSLSCAVKGFYPSDIAVEWESNGQPENNYKTTPP | |
| T366S/L368A/Y407V/Y | VLDSDGSFFLVSKLTVDKSRWQQGNVFSCSVMHEALHNHYTQK | |
| 349C | SLSLSPG | |
| L234A/L235A/P329A | | |
| Without C-terminal | | |
| Lysine | | |
| hIgG1 Hinge Region + | EPKSCDKTHTCPPCPAPEAAGGPSVFLFPPKPKDTLMISRTPE | 217 |
| CH2 Region + CH3 | VTCVVVDVSHEDPEVKFNWYVDGVEVHNAKTKPREEQYNSTYR | |
| Region | VVSVLTVLHQDWLNGKEYKCKVSNKALAAPIEKTISKAKGQPR | |
| Hole | EPQVCTLPPSRDELTKNQVSLSCAVKGFYPSDIAVEWESNGQP | |
| T366S/L368A/Y407V/ | ENNYKTTPPVLDSDGSFFLVSKLTVDKSRWQQGNVFSCSVMHE | |
| Y349C | ALHNHYTQKSLSLSPGK | |
| L234A/L235A/P329A | | |
| With C-terminal Lysine | | |
| hIgG1 Hinge Region + | EPKSCDKTHTCPPCPAPEAAGGPSVFLFPPKPKDTLMISRTPE | 218 |
| CH2 Region + CH3 | VTCVVVDVSHEDPEVKFNWYVDGVEVHNAKTKPREEQYNSTYR | |
| Region | VVSVLTVLHQDWLNGKEYKCKVSNKALAAPIEKTISKAKGQPR | |
| Hole | EPQVCTLPPSRDELTKNQVSLSCAVKGFYPSDIAVEWESNGQP | |
| T366S/L368A/Y407V/ | ENNYKTTPPVLDSDGSFFLVSKLTVDKSRWQQGNVFSCSVMHE | |
| Y349C | ALHNHYTQKSLSLSPG | |
| L234A/L235A/P329A | | |
| Without C-terminal | | |
| Lysine | | |
| hIgG1 CH1+ Hinge | ASTKGPSVFPLAPSSKSTSGGTAALGCLVKDYFPEPVTVSWNS | 219 |
| Region + CH2 Region + | GALTSGVHTFPAVLQSSGLYSLSSVVTVPSSSLGTQTYICNVN | |
| CH3 Region | HKPSNTKVDKKVEPKSCDKTHTCPPCPAPEAAGGPSVFLFPPK | |
| Hole | PKDTLMISRTPEVTCVVVDVSHEDPEVKFNWYVDGVEVHNAKT | |
| T366S/L368A/Y407V/ | KPREEQYNSTYRVVSVLTVLHQDWLNGKEYKCKVSNKALAAPI | |
| Y349C | EKTISKAKGQPREPQVCTLPPSRDELTKNQVSLSCAVKGFYPS | |
| L234A/L235A/P329A | DIAVEWESNGQPENNYKTTPPVLDSDGSFFLVSKLTVDKSRWQ | |
| With C-terminal Lysine | QGNVFSCSVMHEALHNHYTQKSLSLSPGK | |
| hIgG1 CH1 + Hinge | ASTKGPSVFPLAPSSKSTSGGTAALGCLVKDYFPEPVTVSWNS | 220 |
| Region + CH2 Region + | GALTSGVHTFPAVLQSSGLYSLSSVVTVPSSSLGTQTYICNVN | |
| CH3 Region | HKPSNTKVDKKVEPKSCDKTHTCPPCPAPEAAGGPSVFLFPPK | |
| L234A/L235A/P329A | PKDTLMISRTPEVTCVVVDVSHEDPEVKFNWYVDGVEVHNAKT | |
| Hole | KPREEQYNSTYRVVSVLTVLHQDWLNGKEYKCKVSNKALAAPI | |
| T366S/L368A/Y407V/ | EKTISKAKGQPREPQVCTLPPSRDELTKNQVSLSCAVKGFYPS | |
| Y349C | DIAVEWESNGQPENNYKTTPPVLDSDGSFFLVSKLTVDKSRWQ | |
| L234A/L235A/P329A | QGNVFSCSVMHEALHNHYTQKSLSLSPG | |
| Without C-terminal | | |
| Lysine | | |
| hIgG1 CH2 Region + | PCPAPEAAGGPSVFLFPPKPKDTLMISRTPEVTCVVVDVSHED | 221 |
| CH3 Region | PEVKFNWYVDGVEVHNAKTKPREEQYNSTYRVVSVLTVLHQDW | |
| Knob T366W/S354C | LNGKEYKCKVSNKALGAPIEKTISKAKGQPREPQVYTLPPCRD | |
| L234A/L235A/P329A | ELTKNQVSLWCLVKGFYPSDIAVEWESNGQPENNYKTTPPVLD | |
| With C-terminal Lysine | SDGSFFLYSKLTVDKSRWQQGNVESCSVMHEALHNHYTQKSLS | |
| | LSPGK | |
| hIgG1 CH2 Region + | PCPAPEAAGGPSVFLFPPKPKDTLMISRTPEVTCVVVDVSHED | 222 |
| CH3 Region | PEVKFNWYVDGVEVHNAKTKPREEQYNSTYRVVSVLTVLHQDW | |
| Knob T366W/S354C | LNGKEYKCKVSNKALGAPIEKTISKAKGQPREPQVYTLPPCRD | |
| L234A/L235A/P329A | ELTKNQVSLWCLVKGFYPSDIAVEWESNGQPENNYKTTPPVLD | |
| Without C-terminal | SDGSFFLYSKLTVDKSRWQQGNVFSCSVMHEALHNHYTQKSLS | |
| Lysine | LSPG | |
| hIgG1 Partial Hinge | TCPPCPAPEAAGGPSVFLFPPKPKDTLMISRTPEVTCVVVDVS | 223 |
| Region + CH2 Region + | HEDPEVKFNWYVDGVEVHNAKTKPREEQYNSTYRVVSVLTVLH | |
| CH3 Region | QDWLNGKEYKCKVSNKALGAPIEKTISKAKGQPREPQVYTLPP | |
| Knob T366W/S354C | CRDELTKNQVSLWCLVKGFYPSDIAVEWESNGQPENNYKTTPP | |
| L234A/L235A/P329A | VLDSDGSFFLYSKLTVDKSRWQQGNVFSCSVMHEALHNHYTQK | |
| With C-terminal Lysine | SLSLSPGK | |
| hIgG1 Partial Hinge | TCPPCPAPEAAGGPSVFLFPPKPKDTLMISRTPEVTCVVVDVS | 224 |
| Region + CH2 Region + | HEDPEVKFNWYVDGVEVHNAKTKPREEQYNSTYRVVSVLTVLH | |
| CH3 Region | QDWLNGKEYKCKVSNKALGAPIEKTISKAKGQPREPQVYTLPP | |
| Knob T366W/S354C | CRDELTKNQVSLWCLVKGFYPSDIAVEWESNGQPENNYKTTPP | |
| L234A/L235A/P329A | VLDSDGSFFLYSKLTVDKSRWQQGNVFSCSVMHEALHNHYTQK | |
| Without C-terminal | SLSLSPG | |
| Lysine | | |
| hIgG1 Hinge Region + | EPKSCDKTHTCPPCPAPEAAGGPSVFLFPPKPKDTLMISRTPE | 225 |
| CH2 Region + CH3 | VTCVVVDVSHEDPEVKFNWYVDGVEVHNAKTKPREEQYNSTYR | |
| Region | VVSVLTVLHQDWLNGKEYKCKVSNKALGAPIEKTISKAKGQPR | |
| Knob T366W/S354C | EPQVYTLPPCRDELTKNQVSLWCLVKGFYPSDIAVEWESNGQP | |
| L234A/L235A/P329A | ENNYKTTPPVLDSDGSFFLYSKLTVDKSRWQQGNVFSCSVMHE | |
| With C-terminal Lysine | ALHNHYTQKSLSLSPGK | |
| hIgG1 Hinge Region + | EPKSCDKTHTCPPCPAPEAAGGPSVFLFPPKPKDTLMISRTPE | 226 |
| CH2 Region + CH3 | VTCVVVDVSHEDPEVKFNWYVDGVEVHNAKTKPREEQYNSTYR | |
| Region | VVSVLTVLHQDWLNGKEYKCKVSNKALGAPIEKTISKAKGQPR | |
| Knob T366W/S354C | EPQVYTLPPCRDELTKNQVSLWCLVKGFYPSDIAVEWESNGQP | |
| L234A/L235A/P329A | ENNYKTTPPVLDSDGSFFLYSKLTVDKSRWQQGNVFSCSVMHE | |
| Without C-terminal | ALHNHYTQKSLSLSPG | |
| Lysine | | |
| hIgG1 CH1+ Hinge | ASTKGPSVFPLAPSSKSTSGGTAALGCLVKDYFPEPVTVSWNS | 227 |
| Region + CH2 Region + | GALTSGVHTFPAVLQSSGLYSLSSVVTVPSSSLGTQTYICNVN | |
| CH3 Region | HKPSNTKVDKKVEPKSCDKTHTCPPCPAPEAAGGPSVFLFPPK | |
| Knob T366W/S354C | PKDTLMISRTPEVTCVVVDVSHEDPEVKFNWYVDGVEVHNAKT | |
| L234A/L235A/P329A | KPREEQYNSTYRVVSVLTVLHQDWLNGKEYKCKVSNKALGAPI | |
| With C-terminal Lysine | EKTISKAKGQPREPQVYTLPPCRDELTKNQVSLWCLVKGFYPS | |
| | DIAVEWESNGQPENNYKTTPPVLDSDGSFFLYSKLTVDKSRWQ | |
| | QGNVFSCSVMHEALHNHYTQKSLSLSPGK | |
| hIgG1 CH1 + Hinge | ASTKGPSVFPLAPSSKSTSGGTAALGCLVKDYFPEPVTVSWNS | 228 |
| Region + CH2 Region + | GALTSGVHTFPAVLQSSGLYSLSSVVTVPSSSLGTQTYICNVN | |
| CH3 Region | HKPSNTKVDKKVEPKSCDKTHTCPPCPAPEAAGGPSVFLFPPK | |
| Knob T366W/S354C | PKDTLMISRTPEVTCVVVDVSHEDPEVKFNWYVDGVEVHNAKT | |
| L234A/L235A/P329A | KPREEQYNSTYRVVSVLTVLHQDWLNGKEYKCKVSNKALGAPI | |
| Without C-terminal | EKTISKAKGQPREPQVYTLPPCRDELTKNQVSLWCLVKGFYPS | |
| Lysine | DIAVEWESNGQPENNYKTTPPVLDSDGSFFLYSKLTVDKSRWQ | |
| | QGNVFSCSVMHEALHNHYTQKSLSLSPG | |
| hIgG1 CH2 Region + | PCPAPEAAGGPSVFLFPPKPKDTLMISRTPEVTCVVVDVSHED | 229 |
| CH3 Region | PEVKFNWYVDGVEVHNAKTKPREEQYNSTYRVVSVLTVLHQDW | |
| Hole | LNGKEYKCKVSNKALGAPIEKTISKAKGQPREPQVYTLPPSRD | |
| T366S/L368A/Y407V/ | ELTKNQVSLSCAVKGFYPSDIAVEWESNGQPENNYKTTPPVLD | |
| Y349C | SDGSFFLVSKLTVDKSRWQQGNVFSCSVMHEALHNHYTQKSLS | |
| L234A/L235A/P329A | LSPGK | |
| With C-terminal Lysine | | |
| hIgG1 CH2 Region + | PCPAPEAAGGPSVFLFPPKPKDTLMISRTPEVTCVVVDVSHED | 230 |
| CH3 Region | PEVKFNWYVDGVEVHNAKTKPREEQYNSTYRVVSVLTVLHQDW | |
| Hole | LNGKEYKCKVSNKALGAPIEKTISKAKGQPREPQVYTLPPSRD | |
| T366S/L368A/Y407V/ | ELTKNQVSLSCAVKGFYPSDIAVEWESNGQPENNYKTTPPVLD | |
| Y349C | SDGSFFLVSKLTVDKSRWQQGNVFSCSVMHEALHNHYTQKSLS | |
| L234A/L235A/P329A | LSPG | |
| Without C-terminal | | |
| Lysine | | |
| hIgG1 Partial Hinge | TCPPCPAPEAAGGPSVFLFPPKPKDTLMISRTPEVTCVVVDVS | 231 |
| Region + CH2 Region + | HEDPEVKFNWYVDGVEVHNAKTKPREEQYNSTYRVVSVLTVLH | |
| CH3 Region | QDWLNGKEYKCKVSNKALGAPIEKTISKAKGQPREPQVCTLPP | |
| Hole | SRDELTKNQVSLSCAVKGFYPSDIAVEWESNGQPENNYKTTPP | |
| T366S/L368A/Y407V/ | VLDSDGSFFLVSKLTVDKSRWQQGNVFSCSVMHEALHNHYTQK | |
| Y349C | SLSLSPGK | |
| L234A/L235A/P329A | | |
| With C-terminal Lysine | | |
| hIgG1 Partial Hinge | TCPPCPAPEAAGGPSVFLFPPKPKDTLMISRTPEVTCVVVDVS | 232 |
| Region + CH2 Region + | HEDPEVKFNWYVDGVEVHNAKTKPREEQYNSTYRVVSVLTVLH | |
| CH3 Region | QDWLNGKEYKCKVSNKALGAPIEKTISKAKGQPREPQVCTLPP | |
| Hole | SRDELTKNQVSLSCAVKGFYPSDIAVEWESNGQPENNYKTTPP | |
| T366S/L368A/Y407V/ | VLDSDGSFFLVSKLTVDKSRWQQGNVFSCSVMHEALHNHYTQK | |
| Y349C | SLSLSPG | |
| L234A/L235A/P329A | | |
| Without C-terminal | | |
| Lysine | | |
| hIgG1 Hinge Region + | EPKSCDKTHTCPPCPAPEAAGGPSVFLFPPKPKDTLMISRTPE | 233 |
| CH2 Region + CH3 | VTCVVVDVSHEDPEVKFNWYVDGVEVHNAKTKPREEQYNSTYR | |
| Region | VVSVLTVLHQDWLNGKEYKCKVSNKALGAPIEKTISKAKGQPR | |
| Hole | EPQVCTLPPSRDELTKNQVSLSCAVKGFYPSDIAVEWESNGQP | |
| T366S/L368A/Y407V/ | ENNYKTTPPVLDSDGSFFLVSKLTVDKSRWQQGNVESCSVMHE | |
| Y349C | ALHNHYTQKSLSLSPGK | |
| L234A/L235A/P329A | | |
| With C-terminal Lysine | | |
| hIgG1 Hinge Region + | EPKSCDKTHTCPPCPAPEAAGGPSVFLFPPKPKDTLMISRTPE | 234 |
| CH2 Region + CH3 | VTCVVVDVSHEDPEVKFNWYVDGVEVHNAKTKPREEQYNSTYR | |
| Region | VVSVLTVLHQDWLNGKEYKCKVSNKALGAPIEKTISKAKGQPR | |
| Hole | EPQVCTLPPSRDELTKNQVSLSCAVKGFYPSDIAVEWESNGQP | |
| T366S/L368A/Y407V/ | ENNYKTTPPVLDSDGSFFLVSKLTVDKSRWQQGNVFSCSVMHE | |
| Y349C | ALHNHYTQKSLSLSPG | |
| L234A/L235A/P329A | | |
| Without C-terminal | | |
| Lysine | | |
| hIgG1 CH1 + Hinge | ASTKGPSVFPLAPSSKSTSGGTAALGCLVKDYFPEPVTVSWNS | 235 |
| Region + CH2 Region + | GALTSGVHTFPAVLQSSGLYSLSSVVTVPSSSLGTQTYICNVN | |
| CH3 Region | HKPSNTKVDKKVEPKSCDKTHTCPPCPAPEAAGGPSVFLFPPK | |
| Hole | PKDTLMISRTPEVTCVVVDVSHEDPEVKFNWYVDGVEVHNAKT | |
| T366S/L368A/Y407V/ | KPREEQYNSTYRVVSVLTVLHQDWLNGKEYKCKVSNKALGAPI | |
| Y349C | EKTISKAKGQPREPQVCTLPPSRDELTKNQVSLSCAVKGFYPS | |
| L234A/L235A/P329A | DIAVEWESNGQPENNYKTTPPVLDSDGSFFLVSKLTVDKSRWQ | |
| With C-terminal Lysine | QGNVFSCSVMHEALHNHYTQKSLSLSPGK | |
| hIgG1 CH1 + Hinge | ASTKGPSVFPLAPSSKSTSGGTAALGCLVKDYFPEPVTVSWNS | 236 |
| Region + CH2 Region + | GALTSGVHTFPAVLQSSGLYSLSSVVTVPSSSLGTQTYICNVN | |
| CH3 Region | HKPSNTKVDKKVEPKSCDKTHTCPPCPAPEAAGGPSVFLFPPK | |
| L234A/L235A/P329A | PKDTLMISRTPEVTCVVVDVSHEDPEVKFNWYVDGVEVHNAKT | |
| Hole | KPREEQYNSTYRVVSVLTVLHQDWLNGKEYKCKVSNKALGAPI | |
| T366S/L368A/Y407V/Y | EKTISKAKGQPREPQVCTLPPSRDELTKNQVSLSCAVKGFYPS | |
| 349C | DIAVEWESNGQPENNYKTTPPVLDSDGSFFLVSKLTVDKSRWQ | |
| L234A/L235A/P329A | QGNVFSCSVMHEALHNHYTQKSLSLSPG | |
| Without C-terminal | | |
| Lysine | | |

In some embodiments, the amino acid sequence of the first Fc region comprises a tryptophan amino acid residue at position T366, a cysteine amino acid residue at position S354, an alanine amino acid residue at position L234, an alanine amino acid residue at position L235, and an alanine amino acid residue at position P329, EU numbering according to Kabat, and is at least 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 205-212; and the amino acid sequence of the second Fc region comprises a serine amino acid at position T366, an alanine amino acid at position L368, and a valine amino acid at position Y407, a cysteine amino acid residue at position Y349, an alanine amino acid residue at position L234, an alanine amino acid residue at position L235, and an alanine amino acid residue at position P329, EU numbering according to Kabat, and is at least 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 213-220.

In some embodiments, the amino acid sequence of the first Fc region comprises a tryptophan amino acid at position T366, a cysteine amino acid residue at position S354, an alanine amino acid residue at position L234, an alanine amino acid residue at position L235, and an alanine amino acid residue at position P329, EU numbering according to Kabat, and is at least 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 221-228; and the amino acid sequence of the second Fc region comprises a serine amino acid at position T366, an alanine amino acid at position L368, a valine amino acid at position Y407, and a cysteine amino acid at position Y349, a cysteine amino acid residue at position Y349, an alanine amino acid residue at position L234, an alanine amino acid residue at position L235, and an alanine amino acid residue at position P329, EU numbering according to Kabat, and is at least 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 229-236.

In some embodiments, the amino acid sequence of the first Fc region comprises a tryptophan amino acid at position T366, a cysteine amino acid residue at position S354, an alanine amino acid residue at position L234, an alanine amino acid residue at position L235, and an alanine amino acid residue at position P329, EU numbering according to Kabat, and is at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 205; and the amino acid sequence of the second Fc region comprises a serine amino acid at position T366, an alanine amino acid at position L368, and a valine amino acid at position Y407, a cysteine amino acid residue at position Y349, an alanine amino acid residue at position L234, an alanine amino acid residue at position L235, and an alanine amino acid residue at position P329, EU numbering according to Kabat, and is at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 213.

In some embodiments, the amino acid sequence of the first Fc region comprises a tryptophan amino acid at position T366, a cysteine amino acid residue at position S354, an alanine amino acid residue at position L234, an alanine amino acid residue at position L235, and an alanine amino acid residue at position P329, EU numbering according to Kabat, and is at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 206; and the amino acid sequence of the second Fc region comprises a serine amino acid at position T366, an alanine amino acid at position L368, and a valine amino acid at position Y407, a cysteine amino acid residue at position Y349, an alanine amino acid residue at position L234, an alanine amino acid residue at position L235, and an alanine amino acid residue at position P329, EU numbering according to Kabat, and is at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 214.

In some embodiments, the amino acid sequence of the first Fc region comprises a tryptophan amino acid at position T366, a cysteine amino acid residue at position S354, an alanine amino acid residue at position L234, an alanine amino acid residue at position L235, and an alanine amino acid residue at position P329, EU numbering according to Kabat, and is at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 207; and the amino acid sequence of the second Fc region comprises a serine amino acid at position T366, an alanine amino acid at position L368, and a valine amino acid at position Y407, a cysteine amino acid residue at position Y349, an alanine amino acid residue at position L234, an alanine amino acid residue at position L235, and an alanine amino acid residue at position P329, EU numbering according to Kabat, and is at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 215.

In some embodiments, the amino acid sequence of the first Fc region comprises a tryptophan amino acid at position T366, a cysteine amino acid residue at position S354, an alanine amino acid residue at position L234, an alanine amino acid residue at position L235, and an alanine amino acid residue at position P329, EU numbering according to Kabat, and is at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 208; and the amino acid sequence of the second Fc region comprises a serine amino acid at position T366, a cysteine amino acid residue at position Y349, an alanine amino acid residue at position L234, an alanine amino acid residue at position L235, and an alanine amino acid residue at position P329, an alanine amino acid at position L368, and a valine amino acid at position Y407, EU numbering according to Kabat, and is at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 216.

In some embodiments, the amino acid sequence of the first Fc region comprises a tryptophan amino acid at position T366, a cysteine amino acid residue at position S354, an alanine amino acid residue at position L234, an alanine amino acid residue at position L235, and an alanine amino acid residue at position P329, EU numbering according to Kabat, and is at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 209; and the amino acid sequence of the second Fc region comprises a serine amino acid at position T366, an alanine amino acid at position L368, and a valine amino acid at position Y407, a cysteine amino acid residue at position Y349, an alanine amino acid residue at position L234, an alanine amino acid residue at position L235, and an alanine amino acid residue at position P329, EU numbering according to Kabat, and is at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 217.

In some embodiments, the amino acid sequence of the first Fc region comprises a tryptophan amino acid at position T366, a cysteine amino acid residue at position S354, an alanine amino acid residue at position L234, an alanine amino acid residue at position L235, and an alanine amino acid residue at position P329, EU numbering according to Kabat, and is at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 210; and the amino acid sequence of the second Fc region comprises a serine amino acid at position T366, an alanine amino acid at position L368, and a valine amino acid at position Y407, a cysteine amino acid residue at position Y349, an alanine amino acid residue at position L234, an alanine amino acid residue at position L235, and an alanine amino acid residue at position P329, EU numbering according to Kabat, and is at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 218.

In some embodiments, the amino acid sequence of the first Fc region comprises a tryptophan amino acid at position T366, a cysteine amino acid residue at position S354, an alanine amino acid residue at position L234, an alanine amino acid residue at position L235, and an alanine amino acid residue at position P329, EU numbering according to Kabat, and is at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 211; and the amino acid sequence of the second Fc region comprises a serine amino acid at position T366, an alanine amino acid at position L368, and a valine amino acid at position Y407, a cysteine amino acid residue at position Y349, an alanine amino acid residue at position L234, an alanine amino acid residue at position L235, and an alanine amino acid residue at position P329, EU numbering according to Kabat, and is at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 219.

In some embodiments, the amino acid sequence of the first Fc region comprises a tryptophan amino acid at position T366, a cysteine amino acid residue at position S354, an alanine amino acid residue at position L234, an alanine amino acid residue at position L235, and an alanine amino acid residue at position P329, EU numbering according to Kabat, and is at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 212; and the amino acid sequence of the second Fc region comprises a serine amino acid at position T366, an alanine amino acid at position L368, and a valine amino acid at position Y407, a cysteine amino acid residue at position Y349, an alanine amino acid residue at position L234, an alanine amino acid residue at position L235, and an alanine amino acid residue at position P329, EU numbering according to Kabat, and is at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 220.

In some embodiments, the amino acid sequence of the first Fc region comprises a tryptophan amino acid at position T366, a cysteine amino acid residue at position S354, an alanine amino acid residue at position L234, an alanine amino acid residue at position L235, and a glycine amino acid residue at position P329, EU numbering according to Kabat, and is at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 221; and the amino acid sequence of the second Fc region comprises a serine amino acid at position T366, an alanine amino acid at position L368, a cysteine amino acid residue at position Y349, an alanine amino acid residue at position L234, an alanine amino acid residue at position L235, and a glycine amino acid residue at position P329, a valine amino acid at position Y407, and a cysteine amino acid at position Y349, EU numbering according to Kabat, and is at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 229.

In some embodiments, the amino acid sequence of the first Fc region comprises a tryptophan amino acid at position T366, a cysteine amino acid residue at position S354, an alanine amino acid residue at position L234, an alanine amino acid residue at position L235, and a glycine amino acid residue at position P329, EU numbering according to Kabat, and is at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 222; and the amino acid sequence of the second Fc region comprises a serine amino acid at position T366, an alanine amino acid at position L368, a valine amino acid at position Y407, a cysteine amino acid residue at position Y349, an alanine amino acid residue at position L234, an alanine amino acid residue at position L235, and a glycine amino acid residue at position P329, and a cysteine amino acid at position Y349, EU numbering according to Kabat, and is at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 230.

In some embodiments, the amino acid sequence of the first Fc region comprises a tryptophan amino acid at position T366, a cysteine amino acid residue at position S354, an alanine amino acid residue at position L234, an alanine amino acid residue at position L235, and a glycine amino acid residue at position P329, EU numbering according to Kabat, and is at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 223; and the amino acid sequence of the second Fc region comprises a serine amino acid at position T366, an alanine amino acid at position L368, a valine amino acid at position Y407, a cysteine amino acid residue at position Y349, an alanine amino acid residue at position L234, an alanine amino acid residue at position L235, and a glycine amino acid residue at position P329, and a cysteine amino acid at position Y349, EU numbering according to Kabat, and is at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 231.

In some embodiments, the amino acid sequence of the first Fc region comprises a tryptophan amino acid at position T366, a cysteine amino acid residue at position S354, an alanine amino acid residue at position L234, an alanine amino acid residue at position L235, and a glycine amino acid residue at position P329, EU numbering according to Kabat, and is at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 224; and the amino acid sequence of the second Fc region comprises a serine amino acid at position T366, an alanine amino acid at position L368, a valine amino acid at position Y407, and a cysteine amino acid at position Y349, a cysteine amino acid residue at position Y349, an alanine amino acid residue at position L234, an alanine amino acid residue at position L235, and a glycine amino acid residue at position P329, EU numbering according to Kabat, and is at least 85%, 86%, 87%, 88%, 89%, 90%, 91%, 92%, 93%, 94%, 95%, 96%, 97%, 98%, 99%, or 100% identical to the amino acid sequence of set forth in SEQ ID NO: 232.

In some embodiments, the amino acid sequence of the first Fc region comprises a tryptophan amino acid at position T366, a cysteine amino acid residue at position S354, an alanine amino acid residue at position L234, an alanine amino acid residue at position L235, and a glycine amino acid residue at position P329, EU numbering according to Kabat, and is at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 225; and the amino acid sequence of the second Fc region comprises a serine amino acid at position T366, an alanine amino acid at position L368, a valine amino acid at position Y407, and a cysteine amino acid at position Y349, a cysteine amino acid residue at position Y349, an alanine amino acid residue at position L234, an alanine amino acid residue at position L235, and a glycine amino acid residue at position P329, EU numbering according to Kabat, and is at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 233.

In some embodiments, the amino acid sequence of the first Fc region comprises a tryptophan amino acid at position T366, a cysteine amino acid residue at position S354, an alanine amino acid residue at position L234, an alanine amino acid residue at position L235, and a glycine amino acid residue at position P329, EU numbering according to Kabat, and is at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 226; and the amino acid sequence of the second Fc region comprises a serine amino acid at position T366, an alanine amino acid at position L368, a valine amino acid at position Y407, and a cysteine amino acid at position Y349, a cysteine amino acid residue at position Y349, an alanine amino acid residue at position L234, an alanine amino acid residue at position L235, and a glycine amino acid residue at position P329, EU numbering according to Kabat, and is at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 234.

In some embodiments, the amino acid sequence of the first Fc region comprises a tryptophan amino acid at position T366, a cysteine amino acid residue at position S354, an alanine amino acid residue at position L234, an alanine amino acid residue at position L235, and a glycine amino acid residue at position P329, EU numbering according to Kabat, and is at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 227; and the amino acid sequence of the second Fc region comprises a serine amino acid at position T366, an alanine amino acid at position L368, a valine amino acid at position Y407, and a cysteine amino acid at position Y349, a cysteine amino acid residue at position Y349, an alanine amino acid residue at position L234, an alanine amino acid residue at position L235, and an G amino acid residue at position P329, EU numbering according to Kabat, and is at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 235.

In some embodiments, the amino acid sequence of the first Fc region comprises a tryptophan amino acid at position T366, a cysteine amino acid residue at position S354, an alanine amino acid residue at position L234, an alanine amino acid residue at position L235, and a glycine amino acid residue at position P329, EU numbering according to Kabat, and is at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 228; and the amino acid sequence of the second Fc region comprises a serine amino acid at position T366, an alanine amino acid at position L368, a valine amino acid at position Y407, and a cysteine amino acid at position Y349, a cysteine amino acid residue at position Y349, an alanine amino acid residue at position L234, an alanine amino acid residue at position L235, and a glycine amino acid residue at position P329, EU numbering according to Kabat, and is at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 236.

### 5.3.3 Antibody Fusion Proteins

In some embodiments, the fusion protein comprises an antibody (or antigen binding domain thereof) (e.g., a full-length antibody). The antibody component of a fusion protein can act to further target the hIL-12 protein or polypeptide e.g., to a specified cell type expressing a specific cell surface protein. Exemplary antibodies include, but are not limited to, full-length antibodies (or antigen binding domain thereof), scFv, (scFv)₂, Fab, single domain antibodies (e.g., VHH), scFv-Fc, Fab-Fc, single domains antibody-Fc (e.g., VHH-Fc), a dual-affinity re-targeting antibody (DART), minibody, and diabody.

The antibody can comprise any Ig region described herein, e.g., in § 5.3.2. In some embodiments, the antibody comprises one or more Ig region described in § 5.3.2. In some embodiments, the antibody comprises one or more Fc region described in § 5.3.2. In some embodiments, the antibody is a full-length antibody that comprises one or more Ig region described in § 5.3.2. In some embodiments, the antibody is a full-length antibody that comprises one or more Fc region described in § 5.3.2.

In some embodiments, the antibody (or antigen binding domain thereof) specifically binds a human tumor associated antigen (hTAA). Exemplary human tumor associated antigens include, but are not limited to, carbonic anhydrase IX (CAIX), fibroblast activation protein (FAP), mesothelin (MSLN), the A1 domain of tenascin-C (TNC A1), the A2 domain of tenascin-C (TNC A2), the extra domain B of fibronectin (EDB), melanoma-associated chondroitin sulfate proteoglycan (MCSP), MART-1/Melan-A, gp100, dipeptidyl peptidase IV (DPPIV), adenosine deaminase-binding protein (ADAbp), cyclophilin b, colorectal associated antigen (CRC)-C017-1A/GA733, carcinoembryonic antigen (CEA), ETV6, AML1, prostate specific antigen (PSA), prostate-specific membrane antigen (PSMA), MAGE-family of tumor antigens (e.g., MAGE-A1, MAGE-A2, MAGE-A3, MAGE-A4, MAGE-A5, MAGE-A6, MAGE-A7, MAGE-A8, MAGE-A9, MAGE-A10, MAGE-A11, MAGE-A12, MAGE-Xp2 (MAGE-B2), MAGE-Xp3 (MAGE-B3), MAGE-Xp4 (MAGE-B4), MAGE-C1, MAGE-C2, MAGE-C3, MAGE-C4, MAGE-C5), GAGE-family of tumor antigens (e.g., GAGE-1, GAGE-2, GAGE-3, GAGE-4, GAGE-5, GAGE-6, GAGE-7, GAGE-8, GAGE-9), BAGE, RAGE, LAGE-1, NAG, GnT-V, MUM-1, CDK4, tyrosinase, p53, MUC family, p21ras, RCAS1, α-fetoprotein, E-cadherin, α-catenin, β-catenin and γ-catenin, p120ctn, gp100 Pmel117, PRAME, NY-ESO-1, cdc27, adenomatous polyposis coli protein (APC), fodrin, Connexin 37, Ig-idiotype, p15, gp75, GM2 and GD2 gangliosides, viral products such as human papilloma virus proteins, Smad family of tumor antigens, Imp-1, PIA, EBV-encoded nuclear antigen (EBNA)-1, brain glycogen phosphorylase, SSX-1, SSX-2 (HOM-MEL-40), SSX-1, SSX-4, SSX-5, SCP-1 and CT-7, c-erbB-2, Her2, EGFR, IGF-1R, CD23, CD30, CD33, CD40, IL-6R, MCSP, PDGFβR, EpCAM, EGFR variant III, CD19, disialoganglioside GD2, ductal-epithelial mucine, gp36, TAG-72, glioma-associated antigen, β-human chorionic gonadotropin, alphafetoprotein (AFP), lectin-reactive AFP, thyroglobulin, MN-CAIX, human telomerase reverse transcriptase, RU1, RU2, intestinal carboxyl esterase, mut hsp70-2, M-CSF, LAGA-1a, p53, prostein, prostate-carcinoma tumor antigen-1 (PCTA-1), ELF2M, neutrophil elastase, ephrin B2, insulin growth factor (IGF1)-I, IGF-II, IGFI receptor, 5T4, ROR1, Nkp30, NKG2D, membrane spanning 4-domains A1 (MS4A1; CD20); CD22 (SIGLEC2); CD27 (TNFRSF7); TNFRSF8 (CD30); CD33 (SIGLEC3); CD37; CD38; CD40 (TNFRSF5), CD44; CD47; CD48 (SLAMF2); CD52; CD70 (TNFSF7; CD27L); 5′-nucleotidase ecto (NT5E; CD73), ectonucleoside triphosphate diphosphohydrolase 1 (CD39), CD74; CD79B; CD80; CD86; interleukin 3 receptor subunit alpha (IL3RA), prominin 1 (PROM1; CD133); TNFRSF9 (CD137); syndecan 1 (SDC1; CD138); CD200 molecule (CD200); alpha fetoprotein (AFP), BAG cochaperone 6 (BAG6); MET proto-oncogene, receptor tyrosine kinase (MET); KIT proto-oncogene, receptor tyrosine kinase (KIT); C-type lectin domain family 12 member A (CLEC12A; CD371); C-type lectin domain containing 9A (CLEC9A; CD370); cadherin 3 (CDH3); carbonic anhydrase 6 (CA6); carbonic anhydrase 9 (CA9); carcinoembryonic antigen related cell adhesion molecule 3 (CEACAM3); carcinoembryonic antigen related cell adhesion molecule 5 (CEACAM5); carcinoembryonic antigen related cell adhesion molecule 6 (CEACAM6); chorionic somatomammotropin hormone 1 (CSH1); coagulation factor III, tissue factor (F3); collectin subfamily member 10 (COLEC10; CLL1); delta like canonical Notch ligand 3 (DLL3); ectonucleotide pyrophosphatase/phosphodiesterase 3 (ENPP3); ephrin A1 (EFNA1); epidermal growth factor receptor (EGFR; ERBB; HER1); EGFR variant III (EGFRvIII); EPH receptor A2 (EPHA2); epithelial cell adhesion molecule (EPCAM); erb-b2 receptor tyrosine kinase 2 (ERBB2; HER-2/neu); fibroblast growth factor receptor 2 (FGFR2); fibroblast growth factor receptor 3 (FGFR3); folate hydrolase 1 (FOLH1); folate receptor 1 (FOLR1); GD2 ganglioside; glycoprotein NMB (GPNMB; osteoactivin); guanylate cyclase 2C (GUCY2C); human papillomavirus (HPV) E6; HPV E7; major histocompatibility complex (MHC) class I-presented neoantigens, major histocompatibility complex (MHC) class II-presented neoantigens, major histocompatibility complex, class I, E (HLA-E); major histocompatibility complex, class I, F(HLA-F); major histocompatibility complex, class I, G (HLA-G); MHC class I polypeptide-related sequence A (MICA); MHC class I polypeptide-related sequence B (MICB); integrin subunit beta 7 (ITGB7); leukocyte immunoglobulin like receptor B1 (LILRB1; ILT2); leukocyte immunoglobulin like receptor B2 (LILRB2; ILT4); LY6/PLAUR domain containing 3 (LYPD3); glypican 3 (GPC3); KRAS proto-oncogene, GTPase (KRAS); mucin 1 (MUC1) and splice variants thereof (e.g., including MUC1/A, C, D, X, Y, Z and REP); mucin 16 (MUC16; CA125); natural killer cell cytotoxicity receptor 3 ligand 1 (NCR3LG1; B7-H6); necdin, MAGE family member (NDN); nectin cell adhesion molecule 2 (NECTIN2); nectin cell adhesion molecule 4 (NECTIN4); SLIT and NTRK like family member 6 (SLITRK6); promyelocytic leukemia (PML); protein tyrosine kinase 7 (inactive) (PTK7); Poliovirus receptor (PVR) cell adhesion molecule (PVR); SLAM family member 6 (SLAMF6); SLAM family member 7 (SLAMF7); sialic acid binding Ig like lectin 7 (SIGLEC7); sialic acid binding Ig like lectin 9 (SIGLEC9); sialic acid binding Ig like lectin 10 (SIGLEC10); signal regulatory protein alpha (SIRPA) solute carrier family 34 (sodium phosphate), member 2 (SLC34A2); solute carrier family 39 member 6 (SLC39A6); STEAP family member 1 (STEAP1); suppression of tumorigenicity 2 (ST2); TNF receptor superfamily member 4 (TNFRSF4; OX40); TNF superfamily member 9 (TNFSF9; 4-1BB-L, CD137L); TNFRSF10A (DR4, TRAILR1); TNFRSF10B (DR5, TRAILR2); TNFRSF13B (BAFF); TNFRSF17 (BCMA); TNFRSF18 (GITR); transferrin (TF); transforming growth factor beta 1 (TGFB1) and isoforms thereof; triggering receptor expressed on myeloid cells 1 (TREM1); triggering receptor expressed on myeloid cells 2 (TREM2); trophoblast glycoprotein (TPBG); trophinin (TRO); tumor associated calcium signal transducer 2 (TACSTD2); fucosyl GM1; sialyl Lewis adhesion molecule (sLe); and Lewis Y antigen.

In some embodiments, the antibody (or antigen binding domain thereof) specifically binds hCAIX. Exemplary anti-CAIX antibodies, including CDRs, variable heavy chain regions, variable light chain regions, heavy chains, and light chains are described herein, see, e.g., § 5.3.3.1, Tables 2, 3, and 17. In some embodiments, the anti-CAIX antigen binding domain is described herein. In some embodiments, the anti-CAIX antigen binding domain employed is one described in § 5.3.3.1, Tables 2, 3, and 17.

### 5.3.3.1 CAIX Binding Domains

In some embodiments, the fusion proteins described herein comprise an antigen binding domain that specifically binds CAIX, also referred to herein as a hCAIX binding domain or an anti-CAIX domain. In some embodiments, the hCAIX binding domain comprises a Fab, a Fab′, a F(ab′) 2, a F (v), scFv, a (scFv)₂, a scFv-Fc, a (scFv)₂-Fc, a single domain antibody (sdAb), a VHH, a (VHH)₂, a VHH-Fc, or a (VHH)₂-Fc. In some embodiments, the hCAIX binding domain is part of a full-length antibody. In some embodiments, the hCAIX binding domain is part of an Ig Fc fusion (see, e.g., § 5.3.2). In some embodiments, the hCAIX binding domain is a non-antibody antigen binding molecule, e.g., an alternative scaffold known in the art to function as an antigen binding domain, such as e.g., recombinant fibronectin domains.

In some embodiments, the fusion protein is monovalent for hCAIX. In some embodiments, the fusion protein is bivalent for hCAIX. In some embodiments, the fusion protein is trivalent for hCAIX. In some embodiments, the fusion protein is monospecific for a single hCAIX epitope. In some embodiments, the fusion protein is bispecific. In some embodiments, the fusion protein is bispecific, wherein the fusion protein or polypeptide specifically binds a first hCAIX epitope and a second hCAIX epitope, wherein the first and second epitopes are different. In some embodiments, the antibody binds an epitope of the extracellular domain of CAIX (e.g., amino acids 1-193 of SEQ ID NO: 1).

Antibodies that specifically binds hCAIX are known in the art. See, e.g., WO2021000017A1, WO2020226612A1, WO2019204939A1, WO2018234463A1, WO2018157147A1, WO2016199097A1, US20180030147A1, WO2016100980A1, WO2014096163A1, WO2012027493A1, WO2011139375A1, WO2011032973A1, WO2008091798A1, WO2008103327A2, WO2007065027, WO2004002526A1, WO2003100029A2, US20090162382A1, US20050031623A1, US20080176258A1, WO2003048328A2, WO2002063010A2, Chang, D K et al. Human anti-CAIX antibodies mediate immune cell inhibition of renal cell carcinoma in vitro and in a humanized mouse model in vitro, Mol Cancer 14, 119 (2015); Ahlskog, J et al. Human monoclonal antibodies targeting carbonic anhydrase IX for the molecular imaging of hypoxic regions in solid tumours. Br J Cancer 101, 645-657 (2009); Oosterwijk-Wakka J C, Boerman O C, Mulders P F, Oosterwijk E. Application of monoclonal antibody G250 recognizing carbonic anhydrase IX in renal cell carcinoma. Int J Mol Sci. 2013; 14 (6): 11402-11423; De Luca R et al. (2019) A Novel Fully-Human Potency-Matched Dual Cytokine-Antibody Fusion Protein Targets Carbonic Anhydrase IX in Renal Cell Carcinomas. Front. Oncol. 9:1228; Heike M. Petrul et al. Therapeutic Mechanism and Efficacy of the Antibody-Drug Conjugate BAY 79-4620 Targeting Human Carbonic Anhydrase 9, Mol Cancer Ther; 11 (2); 340-9 (2011); the entire contents of each of which is incorporated herein by reference for all purposes.

In some embodiments, the anti-CAIX antibody is girentuximab. Girentuximab is described, for example, in WO2002063010A2, the entire contents of which is incorporated herein by reference for all purposes. In some embodiments, the CAIX binding domain specifically binds to the same epitope as girentuximab. In some embodiments, the antibody is a humanized version of girentuximab. In some embodiments, the TCRVα binding domain is a chimeric version of girentuximab.

The amino acid sequence of an exemplary murine anti-CAIX antibodies, including girentuximab, is provided in Table 17. The CDRs are defined according to the Kabat.

TABLE 17
Amino Acid Sequence of Exemplary Anti-CAIX Binding Domains

| | | | SEQ |
|---|---|---|---|
| Description | Region | Amino Acid Sequence | ID NO |
| A | VH CDR1 | NYYMS | 237 |
| | VH CDR2 | AINSDGGITYYLDTVKG | 238 |
| | VH CDR3 | HRSGYFSMDY | 239 |
| | VL CDR1 | KASQNVVSAVA | 240 |
| | VL CDR2 | SASNRYT | 241 |
| | VL CDR3 | QQYSNYPWT | 242 |
| | VH | EVRLVESGGGLVKPGGSLRLSCAASGFTFSNYYMSWIRQAP | 7 |
| | | EKRLELVSAINSDGGITYYLDTVKGRFTISRDNAKNSLYLQ | |
| | | MNSLRAEDTALFYCARHRSGYFSMDYWGQGTSVTVSS | |
| | VL | EIVMTQSPATLSVSPGERATLSCKASQNVVSAVAWYQQKPG | 12 |
| | | QSPRLLIYSASNRYTGIPARFSGSGSGTEFTLTISSLQSED | |
| | | FAAYYCQQYSNYPWTFGGGTKVEIK | |
| | HC | EVRLVESGGGLVKPGGSLRLSCAASGFTFSNYYMSWIRQAP | 243 |
| | (with C- | EKRLELVSAINSDGGITYYLDTVKGRFTISRDNAKNSLYLQ | |
| | terminal | MNSLRAEDTALFYCARHRSGYFSMDYWGQGTSVTVSSASTK | |
| | lysine) | GPSVFPLAPSSKSTSGGTAALGCLVKDYFPEPVTVSWNSGA | |
| | | LTSGVHTFPAVLQSSGLYSLSSVVTVPSSSLGTQTYICNVN | |
| | | HKPSNTKVDKKVEPKSCDKTHTCPPCPAPELLGGPSVFLFP | |
| | | PKPKDTLMISRTPEVTCVVVDVSHEDPEVKFNWYVDGVEVH | |
| | | NAKTKPREEQYNSTYRVVSVLTVLHQDWLNGKEYKCKVSNK | |
| | | ALPAPIEKTISKAKGQPREPQVYTLPPSRDELTKNQVSLTC | |
| | | LVKGFYPSDIAVEWESNGQPENNYKTTPPVLDSDGSFFLYS | |
| | | KLTVDKSRWQQGNVFSCSVMHEALHNHYTQKSLSLSPGK | |
| | HC | EVRLVESGGGLVKPGGSLRLSCAASGFTFSNYYMSWIRQAP | 244 |
| | (without | EKRLELVSAINSDGGITYYLDTVKGRFTISRDNAKNSLYLQ | |
| | C-terminal | MNSLRAEDTALFYCARHRSGYFSMDYWGQGTSVTVSSASTK | |
| | lysine) | GPSVFPLAPSSKSTSGGTAALGCLVKDYFPEPVTVSWNSGA | |
| | | LTSGVHTFPAVLQSSGLYSLSSVVTVPSSSLGTQTYICNVN | |
| | | HKPSNTKVDKKVEPKSCDKTHTCPPCPAPELLGGPSVFLFP | |
| | | PKPKDTLMISRTPEVTCVVVDVSHEDPEVKFNWYVDGVEVH | |
| | | NAKTKPREEQYNSTYRVVSVLTVLHQDWLNGKEYKCKVSNK | |
| | | ALPAPIEKTISKAKGQPREPQVYTLPPSRDELTKNQVSLTC | |
| | | LVKGFYPSDIAVEWESNGQPENNYKTTPPVLDSDGSFFLYS | |
| | | KLTVDKSRWQQGNVFSCSVMHEALHNHYTQKSLSLSPG | |
| | LC | EIVMTQSPATLSVSPGERATLSCKASQNVVSAVAWYQQKPG | 245 |
| | | QSPRLLIYSASNRYTGIPARFSGSGSGTEFTLTISSLQSED | |
| | | FAAYYCQQYSNYPWTFGGGTKVEIKRTVAAPSVFIFPPSDE | |
| | | QLKSGTASVVCLLNNFYPREAKVQWKVDNALQSGNSQESVT | |
| | | EQDSKDSTYSLSSTLTLSKADYEKHKVYACEVTHQGLSSPV | |
| | | TKSFNRGEC | |
| B | VH CDR1 | NYYMS | 237 |
| (Girentuximab) | VH CDR2 | AINSDGGITYYLDTVKG | 238 |
| | VH CDR3 | HRSGYFSMDY | 239 |
| | VL CDR1 | KASQNVVSAVA | 240 |
| | VL CDR2 | SASNRYT | 241 |
| | VL CDR3 | QQYSNYPWT | 242 |
| | VH | DVKLVESGGGLVKLGGSLKLSCAASGFTFSNYYMSWVRQTP | 246 |
| | | EKRLELVAAINSDGGITYYLDTVKGRFTISRDNAKNTLYLQ | |
| | | MSSLKSEDTALFYCARHRSGYFSMDYWGQGTSVTVSS | |
| | VL | DIVMTQSQRFMSTTVGDRVSITCKASQNVVSAVAWYQQKPG | 247 |
| | | QSPKLLIYSASNRYTGVPDRFTGSGSGTDFTLTISNMQSED | |
| | | LADFFCQQYSNYPWTFGGGTKLEIKR | |
| | HC | DVKLVESGGGLVKLGGSLKLSCAASGFTFSNYYMSWVRQTP | 248 |
| | | EKRLELVAAINSDGGITYYLDTVKGRFTISRDNAKNTLYLQ | |
| | | MSSLKSEDTALFYCARHRSGYFSMDYWGQGTSVTVSSASTK | |
| | | GPSVFPLAPSSKSTSGGTAALGCLVKDYFPEPVTVSWNSGA | |
| | | LTSGVHTFPAVLQSSGLYSLSSVVTVPSSSLGTQTYICNVN | |
| | | HKPSNTKVDKKVEPKSCDKTHTCPPCPAPELLGGPSVFLFP | |
| | | PKPKDTLMISRTPEVTCVVVDVSHEDPEVKFNWYVDGVEVH | |
| | | NAKTKPREEQYNSTYRVVSVLTVLHQDWLNGKEYKCKVSNK | |
| | | ALPAPIEKTISKAKGQPREPQVYTLPPSRDELTKNQVSLTC | |
| | | LVKGFYPSDIAVEWESNGQPENNYKTTPPVLDSDGSFFLYS | |
| | | KLTVDKSRWQQGNVFSCSVMHEALHNHYTQKSLSLSPGK | |
| | LC | DIVMTQSQRFMSTTVGDRVSITCKASQNVVSAVAWYQQKPG | 249 |
| | | QSPKLLIYSASNRYTGVPDRFTGSGSGTDETLTISNMQSED | |
| | | LADFFCQQYSNYPWTFGGGTKLEIKRTVAAPSVFIFPPSDE | |
| | | QLKSGTASVVCLLNNFYPREAKVQWKVDNALQSGNSQESVT | |
| | | EQDSKDSTYSLSSTLTLSKADYEKHKVYACEVTHQGLSSPV | |
| | | TKSFNRGEC | |
| C | VH CDR1 | SHGMA | 250 |
| | VH CDR2 | GISNTGRYTNYGSAVKG | 251 |
| | VH CDR3 | AAVNCVYGCPGSIDA | 252 |
| | VL CDR1 | SGSSGSYG | 253 |
| | VL CDR2 | YNDKRPS | 254 |
| | VL CDR3 | GSADRSGAGI | 255 |
| | VH | AVTLDEPGGGLQTPGGTLSLVCKASGEDISSHGMAWVRQAP | 256 |
| | | GKGLEYVAGISNTGRYTNYGSAVKGRATISRDNGQSTVRLQ | |
| | | LNDLRAEDAGTYFCARAAVNCVYGCPGSIDAWGLGTEV | |
| | | IVSS | |
| | VL | ALTQPSSVSANLGETVEITCSGSSGSYGWYQQKSPGSAPVT | 257 |
| | | VIYYNDKRPSDIPSRFSGSKSGSTGTLTITGVQAEDEAVYY | |
| | | CGSADRSGAGIFGAGTTLTVL | |
| D | VH CDR1 | GFTFTSCYIH | 258 |
| | VH CDR2 | WIYPGNGNTKYNEIFKGR | 259 |
| | VH CDR3 | GDTTANTMDY | 260 |
| | VL CDR1 | RASGNIHNYLA | 261 |
| | VL CDR2 | NTITLAD | 262 |
| | VL CDR3 | QHFWIPFT | 263 |
| | VH | QVQLQQSGPELVKPGASVRISCKASGFTFTSCYIHWMKQRP | 264 |
| | | GQGLEWIGWIYPGNGNTKYNEIFKGRATLTTDKSSSTAYMQ | |
| | | LSSLTSEDSAVYFCARGDTTANTMDYWGQGTSVTVS | |
| | VL | DIQMTQSPASLSASVGETVTITCRASGNIHNYLAWYQQKQG | 265 |
| | | KSPQLLVYNTITLADGVPSRFSGSGSGTQYSLKINSLQPED | |
| | | FGSYYCQHEWIPFTFGAGTKLELK | |
| | HC | QVQLQQSGPELVKPGASVRISCKASGFTFTSCYIHWMKQRP | 266 |
| | | GQGLEWIGWIYPGNGNTKYNEIFKGRATLTTDKSSSTAYMQ | |
| | | LSSLTSEDSAVYFCARGDTTANTMDYWGQGTSVTVSSASTK | |
| | | GPSVFPLAPSSKSTSGGTAALGCLVKDYFPEPVTVSWNSGA | |
| | | LTSGVHTFPAVLQSSGLYSLSSVVTVPSSSLGTQTYICNVN | |
| | | HKPSNTKVDKKVEPKSCDKTHTCPPCPAPELLGGPSVFLFP | |
| | | PKPKDTLMISRTPEVTCVVVDVSHEDPEVKFNWYVDGVEVH | |
| | | NAKTKPREEQYNS | |
| | | TYRVVSVLTVLHQDWLNGKEYKCKVSNKALPAPIEKTISKA | |
| | | KGQPREPQVYTLPPSRDELTKNQVSLTCLVKGFYPSDIAVE | |
| | | WESNGQPENNYKTTPPVLDSDGSFFLYSKLTVDKSRWQQGN | |
| | | VFSCSVMHEALHNHYTQKSLSLSPGK | |
| | LC | DIQMTQSPASLSASVGETVTITCRASGNIHNYLAWYQQKQG | 267 |
| | | KSPQLLVYNTITLADGVPSRESGSGSGTQYSLKINSLQPED | |
| | | FGSYYCQHFWNIPFTFGAGTKLELKRTVAAPSVFIFPPSDE | |
| | | QLKSGTASVVCLLNNFYPREAKVQWKVDNALQSGNSQESVT | |
| | | EQDSKDSTYSLSSTLTLSKADYEKHKVYACEVTHQGLSSPV | |
| | | TKSFNRGEC | |
| E | VH CDR1 | GFTFNTYAMY | 268 |
| | VH CDR2 | RIRSKSNNYAIYYADSVKD | 269 |
| | VH CDR3 | GWDWFAY | 270 |
| | VL CDR1 | RSSQSLVHSNGNTYLH | 271 |
| | VL CDR2 | KVSNRFS | 272 |
| | VL CDR3 | SQNTHVPPT | 273 |
| | VH | EVQLVESGGRLVQPKGSLKLSCAASGFTFNTYAMYWIRQAP | 274 |
| | | GKGLEWVARIRSKSNNYAIYYADSVKDRFTISRDDSQSMLY | |
| | | LQMNNLKTEDTAMYYCVRGWDWFAYWGQGTPVTVSA | |
| | VL | DVVMTQTPLSLPVSLGDQASISCRSSQSLVHSNGNTYLHWY | 275 |
| | | LQKPGQSPKLLIYKVSNRFSGVPDRFSGSGSGTDFTLKISR | |
| | | VEAEDLGVYFCSQNTHVPPTFGGGTKLEIK | |
| | HC | EVQLVESGGRLVQPKGSLKLSCAASGFTFNTYAMYWIRQAP | 276 |
| | | GKGLEWVARIRSKSNNYAIYYADSVKDRFTISRDDSQSMLY | |
| | | LQMNNLKTEDTAMYYCVRGWDWFAYWGQGTPVTVSAASTKG | |
| | | PSVFPLAPSSKSTSGGTAALGCLVKDYFPEPVTVSWNSGAL | |
| | | TSGVHTFPAVLQSSGLYSLSSVVTVPSSSLGTQTYICNVNH | |
| | | KPSNTKVDKKVEPKSCDKTHTCPPCPAPELLGGPSVFLFPP | |
| | | KPKDTLMISRTPEVTCVVVDVSHEDPEVKFNWYVDGVEVHN | |
| | | AKTKPREEQYNSTYRVVSVLTVLHQDWLNGKEYKCKVSNKA | |
| | | LPAPIEKTISKAKGQPREPQVYTLPPSRDELTKNQVSLTCL | |
| | | VKGFYPSDIAVEWESNGQPENNYKTTPPVLDSDGSFFLYSK | |
| | | LTVDKSRWQQGNVFSCSVMHEALHNHYTQKSLSLSPG | |
| | LC | DVVMTQTPLSLPVSLGDQASISCRSSQSLVHSNGNTYLHWY | 277 |
| | | LQKPGQSPKLLIYKVSNRFSGVPDRFSGSGSGTDFTLKISR | |
| | | VEAEDLGVYFCSQNTHVPPTFGGGTKLEIKRTVAAPSVFIF | |
| | | PPSDEQLKSGTASVVCLLNNFYPREAKVQWKVDNALQSGNS | |
| | | QESVTEQDSKDSTYSLSSTLTLSKADYEKHKVYACEVTHQG | |
| | | LSSPVTKSFNRGEC | |
| F | VH CDR1 | GYTFTNYGMN | 278 |
| | VH CDR2 | WINTYTGEPTYADDFKG | 279 |
| | VH CDR3 | GGIATPTSY | 280 |
| | VL CDR1 | KSSQSLLDSDGKTYLN | 281 |
| | VL CDR2 | LVSKLDS | 282 |
| | VL CDR3 | CQGTHFPW | 283 |
| | VH | QIQLVQSGPELKKPGETVKISCKASGYTFTNYGMNWVQQAP | 284 |
| | | GKGLKWMGWINTYTGEPTYADDFKGRFAFSLETSASTAYLQ | |
| | | INNLKNEDMATYFCARGGIATPTSYWGQGTTLTVSS | |
| | VL | DVVMTQTPLTLSVTIGQPASISCKSSQSLLDSDGKTYLNWL | 285 |
| | | LQRPGQSPKRLIYLVSKLDSGVPDRFTGSGSGTDFTLKISR | |
| | | VEAEDLGVYYCCQGTHFPWTFGGGTKLEIK | |
| | HC | QIQLVQSGPELKKPGETVKISCKASGYTFTNYGMNWVQQAP | 286 |
| | | GKGLKWMGWINTYTGEPTYADDFKGRFAFSLETSASTAYLQ | |
| | | INNLKNEDMATYFCARGGIATPTSYWGQGTTLTVSSASTKG | |
| | | PSVFPLAPSSKSTSGGTAALGCLVKDYFPEPVTVSWNSGAL | |
| | | TSGVHTFPAVLQSSGLYSLSSVVTVPSSSLGTQTYICNVNH | |
| | | KPSNTKVDKKVEPKSCDKTHTCPPCPAPELLGGPSVFLFPP | |
| | | KPKDTLMISRTPEVTCVVVDVSHEDPEVKFNWYVDGVEVHN | |
| | | AKTKPREEQYNSTYRVVSVLTVLHQDWLNGKEYKCKVSNKA | |
| | | LPAPIEKTISKAKGQPREPQVYTLPPSRDELTKNQVSLTCL | |
| | | VKGFYPSDIAVEWESNGQPENNYKTTPPVLDSDGSFFLYSK | |
| | | LTVDKSRWQQGNVFSCSVMHEALHNHYTQKSLSLSPG | |
| | LC | DVVMTQTPLTLSVTIGQPASISCKSSQSLLDSDGKTYLNWL | 287 |
| | | LQRPGQSPKRLIYLVSKLDSGVPDRFTGSGSGTDFTLKISR | |
| | | VEAEDLGVYYCCQGTHEPWTFGGGTKLEIKRTVAAPSVFIF | |
| | | PPSDEQLKSGTASVVCLLNNFYPREAKVQWKVDNALQSGNS | |
| | | QESVTEQDSKDSTYSLSSTLTLSKADYEKHKVYACEVTHQG | |
| | | LSSPVTKSFNRGEC | |

In some embodiments the hCAIX binding domain comprises a VH that comprises: VH CDR1, VH CDR2, and VH CDR3. In some embodiments, the HCAIX binding domain comprises a VL that comprises: VL CDR1, VL CDR2, and VL CDR3. In some embodiments the hCAIX binding domain comprises a VH that comprises a VH CDR1, a VH CDR2, and a VH CDR3; a VL that comprises VL CDR1, VL CDR2, and VL CDR3.

In some embodiments, the amino acid sequence of the VH CDR1, VH CDR2, VH CDR3, VL CDR1, VL CDR2, and VL CDR3 each comprises or consists of the amino acid sequence of a VH CDR1, VH CDR2, VH CDR3, VL CDR1, VL CDR2, and VL CDR3 of an antibody set forth in Table 17.

In some embodiments, the amino acid sequence of VH CDR1 comprises the amino acid sequence of SEQ ID NO: 237, or the amino acid sequence of SEQ ID NO: 237 with 1, 2, or 3 amino acid modifications (e.g., substitution, deletion, addition, etc.); the amino acid sequence of VH CDR2 comprises the amino acid sequence of SEQ ID NO: 238, or the amino acid sequence of SEQ ID NO: 238 with 1, 2, or 3 amino acid modifications (e.g., substitution, deletion, addition, etc.); the amino acid sequence of VH CDR3 comprises the amino acid sequence of SEQ ID NO: 239, or the amino acid sequence of SEQ ID NO: 239 with 1, 2, or 3 amino acid modifications (e.g., substitution, deletion, addition, etc.); the amino acid sequence of VL CDR1 comprises the amino acid sequence of SEQ ID NO: 240, or the amino acid sequence of SEQ ID NO: 240 with 1, 2, or 3 amino acid modifications (e.g., substitution, deletion, addition, etc.); the amino acid sequence of VL CDR2 comprises the amino acid sequence of SEQ ID NO: 241 or the amino acid sequence of SEQ ID NO: 241 with 1, 2, or 3 amino acid modifications (e.g., substitution, deletion, addition, etc.); and the amino acid sequence of VL CDR3 comprises the amino acid sequence of SEQ ID NO: 242, or the amino acid sequence of SEQ ID NO: 242 with 1, 2, or 3 amino acid modifications (e.g., substitution, deletion, addition, etc.).

In some embodiments, the amino acid sequence of VH CDR1 comprises the amino acid sequence of SEQ ID NO: 250, or the amino acid sequence of SEQ ID NO: 250 with 1, 2, or 3 amino acid modifications (e.g., substitution, deletion, addition, etc.); the amino acid sequence of VH CDR2 comprises the amino acid sequence of SEQ ID NO: 251, or the amino acid sequence of SEQ ID NO: 251 with 1, 2, or 3 amino acid modifications (e.g., substitution, deletion, addition, etc.); the amino acid sequence of VH CDR3 comprises the amino acid sequence of SEQ ID NO: 252, or the amino acid sequence of SEQ ID NO: 252 with 1, 2, or 3 amino acid modifications (e.g., substitution, deletion, addition, etc.); the amino acid sequence of VL CDR1 comprises the amino acid sequence of SEQ ID NO: 253, or the amino acid sequence of SEQ ID NO: 253 with 1, 2, or 3 amino acid modifications (e.g., substitution, deletion, addition, etc.); the amino acid sequence of VL CDR2 comprises the amino acid sequence of SEQ ID NO: 254 or the amino acid sequence of SEQ ID NO: 254 with 1, 2, or 3 amino acid modifications (e.g., substitution, deletion, addition, etc.); and the amino acid sequence of VL CDR3 comprises the amino acid sequence of SEQ ID NO: 255, or the amino acid sequence of SEQ ID NO: 255 with 1, 2, or 3 amino acid modifications (e.g., substitution, deletion, addition, etc.).

In some embodiments, the amino acid sequence of VH CDR1 comprises the amino acid sequence of SEQ ID NO: 258, or the amino acid sequence of SEQ ID NO: 258 with 1, 2, or 3 amino acid modifications (e.g., substitution, deletion, addition, etc.); the amino acid sequence of VH CDR2 comprises the amino acid sequence of SEQ ID NO: 259, or the amino acid sequence of SEQ ID NO: 259 with 1, 2, or 3 amino acid modifications (e.g., substitution, deletion, addition, etc.); the amino acid sequence of VH CDR3 comprises the amino acid sequence of SEQ ID NO: 260, or the amino acid sequence of SEQ ID NO: 260 with 1, 2, or 3 amino acid modifications (e.g., substitution, deletion, addition, etc.); the amino acid sequence of VL CDR1 comprises the amino acid sequence of SEQ ID NO: 261, or the amino acid sequence of SEQ ID NO: 261 with 1, 2, or 3 amino acid modifications (e.g., substitution, deletion, addition, etc.); the amino acid sequence of VL CDR2 comprises the amino acid sequence of SEQ ID NO: 262 or the amino acid sequence of SEQ ID NO: 262 with 1, 2, or 3 amino acid modifications (e.g., substitution, deletion, addition, etc.); and the amino acid sequence of VL CDR3 comprises the amino acid sequence of SEQ ID NO: 263, or the amino acid sequence of SEQ ID NO: 263 with 1, 2, or 3 amino acid modifications (e.g., substitution, deletion, addition, etc.).

In some embodiments, the amino acid sequence of VH CDR1 comprises the amino acid sequence of SEQ ID NO: 268, or the amino acid sequence of SEQ ID NO: 268 with 1, 2, or 3 amino acid modifications (e.g., substitution, deletion, addition, etc.); the amino acid sequence of VH CDR2 comprises the amino acid sequence of SEQ ID NO: 269, or the amino acid sequence of SEQ ID NO: 269 with 1, 2, or 3 amino acid modifications (e.g., substitution, deletion, addition, etc.); the amino acid sequence of VH CDR3 comprises the amino acid sequence of SEQ ID NO: 270, or the amino acid sequence of SEQ ID NO: 270 with 1, 2, or 3 amino acid modifications (e.g., substitution, deletion, addition, etc.); the amino acid sequence of VL CDR1 comprises the amino acid sequence of SEQ ID NO: 271, or the amino acid sequence of SEQ ID NO: 271 with 1, 2, or 3 amino acid modifications (e.g., substitution, deletion, addition, etc.); the amino acid sequence of VL CDR2 comprises the amino acid sequence of SEQ ID NO: 272 or the amino acid sequence of SEQ ID NO: 272 with 1, 2, or 3 amino acid modifications (e.g., substitution, deletion, addition, etc.); and the amino acid sequence of VL CDR3 comprises the amino acid sequence of SEQ ID NO: 273, or the amino acid sequence of SEQ ID NO: 273 with 1, 2, or 3 amino acid modifications (e.g., substitution, deletion, addition, etc.).

In some embodiments, the amino acid sequence of VH CDR1 comprises the amino acid sequence of SEQ ID NO: 278, or the amino acid sequence of SEQ ID NO: 278 with 1, 2, or 3 amino acid modifications (e.g., substitution, deletion, addition, etc.); the amino acid sequence of VH CDR2 comprises the amino acid sequence of SEQ ID NO: 279, or the amino acid sequence of SEQ ID NO: 279 with 1, 2, or 3 amino acid modifications (e.g., substitution, deletion, addition, etc.); the amino acid sequence of VH CDR3 comprises the amino acid sequence of SEQ ID NO: 280, or the amino acid sequence of SEQ ID NO: 280 with 1, 2, or 3 amino acid modifications (e.g., substitution, deletion, addition, etc.); the amino acid sequence of VL CDR1 comprises the amino acid sequence of SEQ ID NO: 281, or the amino acid sequence of SEQ ID NO: 281 with 1, 2, or 3 amino acid modifications (e.g., substitution, deletion, addition, etc.); the amino acid sequence of VL CDR2 comprises the amino acid sequence of SEQ ID NO: 282 or the amino acid sequence of SEQ ID NO: 282 with 1, 2, or 3 amino acid modifications (e.g., substitution, deletion, addition, etc.); and the amino acid sequence of VL CDR3 comprises the amino acid sequence of SEQ ID NO: 283, or the amino acid sequence of SEQ ID NO: 283 with 1, 2, or 3 amino acid modifications (e.g., substitution, deletion, addition, etc.).

In some embodiments, the hCAIX binding domain comprises a VH and a VL.

In some embodiments, the amino acid sequence of the VH comprises or consists of an amino acid sequence at least 85%, 86%, 87%, 88%, 89%, 90%, 91%, 92%, 93%, 94%, 95%, 96%, 97%, 98%, 99%, or 100% identical to the amino acid sequence of any VH polypeptide set forth in Table 17; and the amino acid sequence of the VL comprises or consists of an amino acid sequence at least 85%, 86%, 87%, 88%, 89%, 90%, 91%, 92%, 93%, 94%, 95%, 96%, 97%, 98%, 99%, or 100% identical to the amino acid sequence of any VL polypeptide set forth in Table 17.

In some embodiments, the amino acid sequence of the VH comprises or consists of an amino acid sequence at least 85%, 86%, 87%, 88%, 89%, 90%, 91%, 92%, 93%, 94%, 95%, 96%, 97%, 98%, 99%, or 100% identical to the amino acid sequence of any one of SEQ ID NOS: 7, 246, 256, 264, 274, or 284.

In some embodiments, the amino acid sequence of the VL comprises or consists of an amino acid sequence at least 85%, 86%, 87%, 88%, 89%, 90%, 91%, 92%, 93%, 94%, 95%, 96%, 97%, 98%, 99%, or 100% identical to the amino acid sequence of any one of SEQ ID NOS: 12, 247, 257, 265, 275, or 285.

In some embodiments, the amino acid sequence of the VH comprises or consists of an amino acid sequence at least 85%, 86%, 87%, 88%, 89%, 90%, 91%, 92%, 93%, 94%, 95%, 96%, 97%, 98%, 99%, or 100% identical to the amino acid sequence of SEQ ID NO: 7; and the amino acid sequence of the VL comprises or consists of an amino acid sequence at least 85%, 86%, 87%, 88%, 89%, 90%, 91%, 92%, 93%, 94%, 95%, 96%, 97%, 98%, 99%, or 100% identical to the amino acid sequence of SEQ ID NO: 12.

In some embodiments, the anti-CAIX antibody comprises a heavy chain that comprises an amino acid sequence at least 90%, 91%, 92%, 93%, 94%, 95%, 96%, 97%, 98%, 99%, or 100% identical to the amino acid sequence of SEQ ID NO: 246; and a light chain that comprises an amino acid sequence at least 90%, 91%, 92%, 93%, 94%, 95%, 96%, 97%, 98%, 99%, or 100% identical to the amino acid sequence of SEQ ID NO: 247.

In some embodiments, the anti-CAIX antibody comprises a heavy chain that comprises an amino acid sequence at least 90%, 91%, 92%, 93%, 94%, 95%, 96%, 97%, 98%, 99%, or 100% identical to the amino acid sequence of SEQ ID NO: 256; and a light chain that comprises an amino acid sequence at least 90%, 91%, 92%, 93%, 94%, 95%, 96%, 97%, 98%, 99%, or 100% identical to the amino acid sequence of SEQ ID NO: 257.

In some embodiments, the anti-CAIX antibody comprises a heavy chain that comprises an amino acid sequence at least 90%, 91%, 92%, 93%, 94%, 95%, 96%, 97%, 98%, 99%, or 100% identical to the amino acid sequence of SEQ ID NO: 264; and a light chain that comprises an amino acid sequence at least 90%, 91%, 92%, 93%, 94%, 95%, 96%, 97%, 98%, 99%, or 100% identical to the amino acid sequence of SEQ ID NO: 265.

In some embodiments, the anti-CAIX antibody comprises a heavy chain that comprises an amino acid sequence at least 90%, 91%, 92%, 93%, 94%, 95%, 96%, 97%, 98%, 99%, or 100% identical to the amino acid sequence of SEQ ID NO: 274; and a light chain that comprises an amino acid sequence at least 90%, 91%, 92%, 93%, 94%, 95%, 96%, 97%, 98%, 99%, or 100% identical to the amino acid sequence of SEQ ID NO: 275.

In some embodiments, the anti-CAIX antibody comprises a heavy chain that comprises an amino acid sequence at least 90%, 91%, 92%, 93%, 94%, 95%, 96%, 97%, 98%, 99%, or 100% identical to the amino acid sequence of SEQ ID NO: 284; and a light chain that comprises an amino acid sequence at least 90%, 91%, 92%, 93%, 94%, 95%, 96%, 97%, 98%, 99%, or 100% identical to the amino acid sequence of SEQ ID NO: 285.

In some embodiments, the hCAIX binding domain comprises a VH, VL, or VH and VL of a humanized anti-CAIX antibody described herein (e.g., see, § 5.1.1, and Table 2). The full contents of § 5.1.1 is incorporated by reference into this § 5.3.3.1.

For example, in some embodiments, the amino acid sequence of the VH is at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 7; and the amino acid sequence of the VL is at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 12.

For example, in some embodiments, the amino acid sequence of the VH is at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 7; and the amino acid sequence of the VL is at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 15.

### 5.3.4 Linkers

The components of a fusion protein described herein can be directly operably connected (e.g., through a peptide bond) or indirectly operably connected (e.g., through a peptide linker) (e.g., hIL-12 can be operably connected to a full-length directly or indirectly). In some embodiments, the components of a fusion protein described herein (e.g., hIL-12 (or subunits thereof) and an antibody (e.g., a full-length antibody)) are directly operably connected through a peptide bond. In some embodiments, the components of a fusion protein described herein (e.g., hIL-12 (or subunits thereof) and an antibody (e.g., a full-length antibody)) are indirectly operably connected through a peptide linker.

In some embodiments, the peptide linker is one or any combination of a cleavable linker, a non-cleavable linker, a flexible linker, a rigid linker, a helical linker, and/or a non-helical linker.

In some embodiments, the peptide linker comprises from or from about 2-30, 5-30, 10-30, 15-30, 20-30, 25-30, 2-25, 5-25, 10-25, 15-25, 20-25, 2-20, 5-20, 10-20, 15-20, 2-15, 5-15, 10-15, 2-10, or 5-10 amino acid residues. In some embodiments, the peptide linker comprises at least about 2, 3, 4, 5, 6, 7, 8, 9, 10, 11, 12, 13, 14, 15, 16, 17, 18, 19, 20, 21, 22, 23, 24, 25, 26, 27, 28, 29, or 30 amino acid residues. In some embodiments, the linker comprises or consists of about 2, 3, 4, 5, 6, 7, 8, 9, 10, 11, 12, 13, 14, 15, 16, 17, 18, 19, 20, 21, 22, 23, 24, 25, 26, 27, 28, 29, or 30 amino acid residues. In some embodiments, the linker comprises or consists of no more than about 2, 3, 4, 5, 6, 7, 8, 9, 10, 11, 12, 13, 14, 15, 16, 17, 18, 19, 20, 21, 22, 23, 24, 25, 26, 27, 28, 29, or 30 amino acid residues.

In some embodiments, the amino acid sequence of the peptide linker comprises or consists of glycine, serine, or both glycine and serine amino acid residues. In some embodiments, the amino acid sequence of the peptide linker comprises or consists of glycine, serine, and proline amino acid residues.

In some embodiments, the linker comprises a coil domain, see, e.g., U.S. Pat. No. 9,284,375, the full contents of which is incorporated herein by reference for all purposes. In some embodiments, the coil domain is selected from those disclosed in U.S. Pat. No. 9,284,375. In some embodiments, the coil domain comprises an E-Coil domain. In some embodiments, the E-coil domain comprises the amino acid sequence of SEQ ID NO: 302, or the amino acid sequence of SEQ ID NO: 302 comprising 1, 2, or 3 amino acid modifications. In some embodiments, the coil domain is a K-Coil domain. In some embodiments, the K-coil domain comprises the amino acid sequence of SEQ ID NO: 303, the amino acid sequence of SEQ ID NO: 303 comprising 1, 2, or 3 amino acid modifications.

The amino acid sequence of exemplary peptide linkers and coil domains, which can be incorporated in one or more of the embodiments described herein (e.g., fusion proteins and polypeptide), is set provided in Table 18.

TABLE 18
The Amino Acid Sequence of Exemplary Peptide
Linkers

| | | SEQ |
|---|---|---|
| | | ID |
| Description | Amino Acid Sequence | NO |
| Linker A | GGGS | 66 |
| Linker B | GGGSGGGS | 67 |
| Linker C | GGGSGGGSGGGS | 68 |
| Linker D | GGGSGGGSGGGSGGGS | 69 |
| Linker E | GGGGS | 70 |
| Linker F | GGGGSGGGGS | 71 |
| Linker G | GGGGSGGGGSGGGGS | 72 |
| Linker H | GGGGSGGGGSGGGGSGGGGS | 73 |
| Linker I | GGGGGGGS | 74 |
| Linker J | GGGGGGGGGGGGGGS | 75 |
| Linker K | GGGGGGGSGGGGGGGSGGGGGGGS | 76 |
| Linker L | GGGGGGGSGGGGGGGSGGGGGGGGGGGGGGS | 77 |
| Linker M | SGGGG | 78 |
| Linker N | SGGGGSGGGG | 79 |
| Linker O | SGGGGSGGGGSGGGG | 80 |
| Linker P | SGGGGSGGGGSGGGGSGGGG | 81 |
| Linker EE | GGGGGGS | 369 |
| Linker Q | LKGKKG | 288 |
| Linker R | LKGKKGC | 289 |
| Linker S | LQVYYRM | 290 |
| Linker T | LQVYYRMC | 291 |
| Linker U | VEPKSCGGGS | 292 |
| Linker V | VQVHYRM | 293 |
| Linker W | YLYLRARV | 294 |
| Linker X | FNRGECGGGS | 295 |
| Linker Y | LEGEEG | 296 |
| Linker Z | LEGEEGC | 297 |
| Linker AA | LGEEG | 298 |
| Linker BB | LGEEGC | 299 |
| Linker CC | LGKKG | 300 |
| Linker DD | LGKKGC | 301 |
| “E-coil” | EVAALEKEVAALEKEVAALEKEVAALEK | 302 |
| Positively | | |
| charged | | |
| “K-coil” | KVAALKEKVAALKEKVAALKEKVAALKE | 303 |
| Negatively | | |
| charged | | |

In some embodiments, the amino acid sequence of the peptide linker comprises or consists of the amino acid sequence of any one of the linkers set forth in Table 18. In some embodiments, the amino acid sequence of the peptide linker comprises or consists of the amino acid sequence of any one of the linkers set forth in Table 18, and further comprises 1 or more but less than 15% (less than 12%, less than 10%, less than 8%), amino acid modifications (e.g., amino acid substitutions, deletions, or additions). In some embodiments, the amino acid sequence of the peptide linker comprises or consists of the amino acid sequence of any one of the linkers set forth in Table 18, comprising 1, 2, or 3 amino acid modifications (e.g., substitutions, deletions, additions). In some embodiments, the amino acid sequence of the peptide linker comprises or consists of the amino acid sequence of any one of the linkers set forth in Table 18, and further comprises 1 or more but less than 15% (less than 12%, less than 10%, less than 8%), amino acid substitutions. In some embodiments, the amino acid sequence of the peptide linker comprises or consists of the amino acid sequence of any one of the linkers set forth in Table 18, comprising 1, 2, or 3 amino acid substitutions.

In some embodiments, the amino acid sequence of the peptide linker comprises or consists of the amino acid sequence of any one of SEQ ID NOS: 66-81 or 288-303. In some embodiments, the amino acid sequence of the peptide linker comprises or consists of the amino acid sequence of any one of SEQ ID NOS: 66-81 or 288-303, and further comprises 1 or more but less than 15% (less than 12%, less than 10%, less than 8%), amino acid modifications (e.g., amino acid substitutions, deletions, or additions). In some embodiments, the amino acid sequence of the peptide linker comprises or consists of the amino acid sequence of any one of SEQ ID NOS: 66-81, 288-303, or 369 comprising 1, 2, or 3 amino acid modifications (e.g., substitutions, deletions, additions). In some embodiments, the amino acid sequence of the peptide linker comprises or consists of the amino acid sequence of any one of SEQ ID NOS: 66-81, 288-303, or 369 and further comprises 1 or more but less than 15% (less than 12%, less than 10%, less than 8%), amino acid substitutions. In some embodiments, the amino acid sequence of the peptide linker comprises or consists of the amino acid sequence of any one of SEQ ID NOS: 66-81, 288-303, or 369 comprising 1, 2, or 3 amino acid substitutions.

In some embodiments, the amino acid sequence of the peptide linker comprises or consists of the amino acid sequence of SEQ ID NO: 72, and further comprises 1 or more but less than 15% (less than 12%, less than 10%, less than 8%), amino acid substitutions. In some embodiments, the amino acid sequence of the peptide linker comprises or consists of the amino acid sequence of SEQ ID NO: 72. In some embodiments, the amino acid sequence of the peptide linker comprises or consists of the amino acid sequence of any one of SEQ ID NO: 72, comprising 1, 2, or 3 amino acid modifications (e.g., substitutions, additions, deletions). In some embodiments, the amino acid sequence of the peptide linker comprises or consists of the amino acid sequence of any one of SEQ ID NO: 72, comprising 1, 2, or 3 amino acid substitutions.

### 5.3.5 Structure & Orientation

The components of a fusion protein described herein can be arranged in any configuration or order as long as each component of the fusion protein or polypeptide maintains the ability to mediate its function. For example, the hIL-12 protein will maintain the ability to bind the hIL-12R. For example, in embodiments, wherein the fusion protein comprises an antibody (e.g., an anti-hCAIX antibody), the antibody will maintain the ability to bind its cognate antigen (e.g., hCAIX).

### 5.3.6 Exemplary Structures & Orientation

### 5.3.6.1 Exemplary Full-Length Antibody Fusion Proteins

In some embodiments, the heterologous moiety is a full-length antibody (e.g., an anti-hCAIX full-length antibody) comprising (i) a first Ig light chain comprising from N- to C-terminus a light chain variable region (VL) region and a light chain constant region (CL) region; (ii) a first Ig heavy chain comprising from N- to C-terminus a heavy chain variable region (VH) region, a CH1 region, a hinge region, a CH2 region, and a CH3 region; (iii) a second Ig heavy chain comprising from N- to C-terminus a VH region, a CH1 region, a hinge region, a CH2 region, and a CH3 region; (iv) a second Ig light chain comprising from N- to C-terminus a VL region and a VH region; wherein said first light chain and said first heavy chain associate to form a first antigen binding domain; wherein said second light chain and said second heavy chain associate to form a second antigen binding domain; and wherein said first heavy chain and said second heavy chain associate to form a dimer.

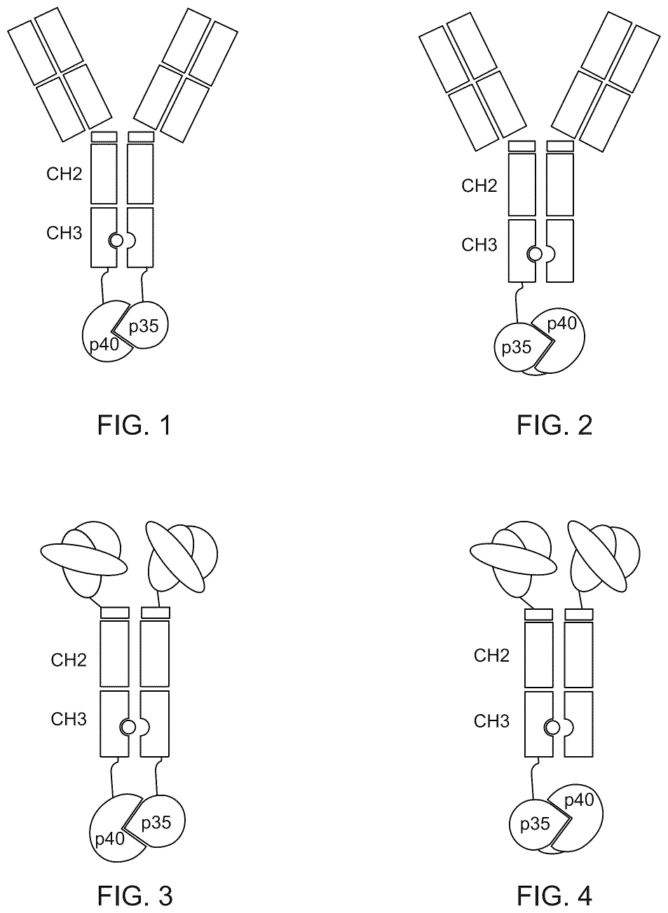

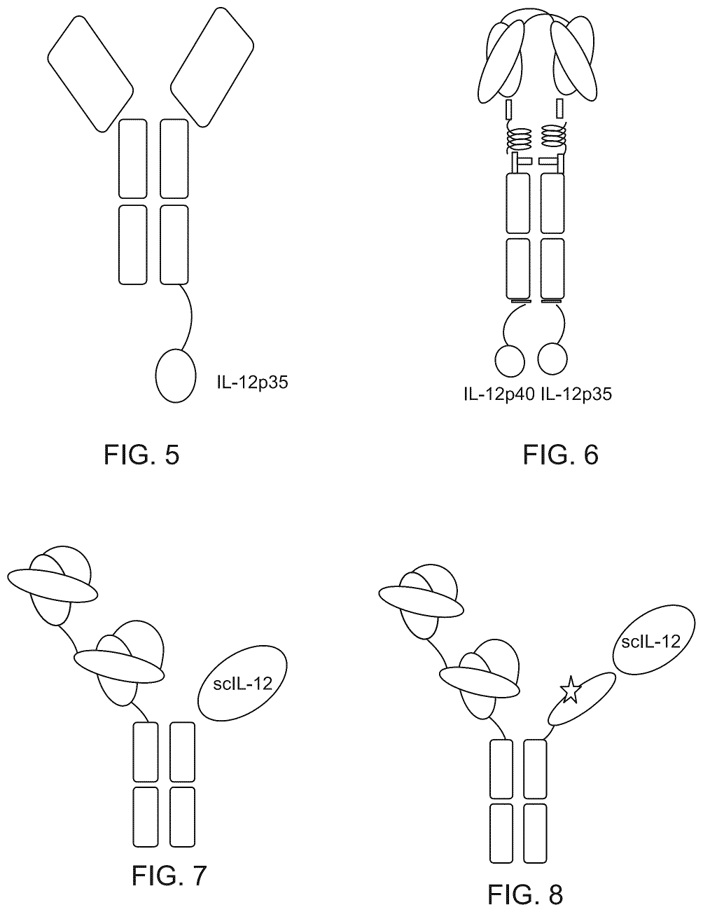

In some embodiments, the N-terminus of the hIL-12p35 polypeptide of the hIL-12 protein is operably connected to the C-terminus of the CH3 region of the first Ig heavy chain of the full-length antibody and the N-terminus of the hIL-12p40 polypeptide of the hIL-12 protein is operably connected to the C-terminus of the CH3 region of the second Ig heavy chain of the full-length antibody (see, e.g., FIG. 1). In some embodiments, the N-terminus of the hIL-12p35 polypeptide of the hIL-12 protein is directly operably connected to the C-terminus of the CH3 region of the first Ig heavy chain of the full-length antibody through a peptide bond and the N-terminus of the hIL-12p40 polypeptide of the hIL-12 protein is directly operably connected to the C-terminus of the CH3 region of the second Ig heavy chain of the full-length antibody through a peptide bond. In some embodiments, the N-terminus of the hIL-12p35 polypeptide of the hIL-12 protein is indirectly operably connected to the C-terminus of the CH3 region of the first Ig heavy chain of the full-length antibody through a peptide linker and the N-terminus of the hIL-12p40 polypeptide of the hIL-12 protein is indirectly operably connected to the C-terminus of the CH3 region of the second Ig heavy chain of the full-length antibody through a peptide linker. In some embodiments, (a) the C-terminus of the CH3 region of the first Ig heavy chain of the full-length antibody is operably connected to the N-terminus of a peptide linker, the C-terminus of the peptide linker is operably connected to the N-terminus of the hIL-12p35 polypeptide; and (b) the C-terminus of the CH3 region of the second Ig heavy chain of the full-length antibody is operably connected to the N-terminus of a peptide linker, the C-terminus of the peptide linker is operably connected to the N-terminus of the hIL-12p35 polypeptide. Exemplary peptide linkers are described herein, see, e.g., § 5.3.4, Table 18, SEQ ID NOS: 66-81, 288-303, or 369.

In some embodiments, the fusion protein comprises a full-length antibody (e.g., an anti-hCAIX full-length antibody) that comprises a) a first polypeptide that comprises from N- to C-terminus a first light chain variable region (VL), and a first light chain constant region (CL); a second polypeptide that comprises from N- to C-terminus a first heavy chain variable region (VH), a first heavy chain constant region (CH), an optional peptide linker (e.g., a peptide linker described herein), and a hIL-12p35; a third polypeptide that comprises from N- to C-terminus a second VH, a second CH, an optional peptide linker (e.g., a peptide linker described herein), and a hIL-12p40; and a fourth polypeptide that comprises from N- to C-terminus a second VL and a second CL; wherein the first polypeptide and second polypeptide associate to form a first antigen binding domain (e.g., that specifically binds hCAIX) and the third polypeptide and the fourth polypeptide associate to form a second antigen binding domain that specifically binds hCAIX. In some embodiments, the first CH region comprises from N- to C-terminus a CH1 region, a hinge region, a CH2 region, and a CH3 region. In some embodiments, the second CH region comprises from N- to C-terminus a CH1 region, a hinge region, a CH2 region, and a CH3 region. In some embodiments, the first polypeptide and the second polypeptide are connected via at least one disulfide bond. In some embodiments, the second polypeptide and the third polypeptide are connected via at least one disulfide bond. In some embodiments, the third polypeptide and the fourth polypeptide are connected via at least one disulfide bond. In some embodiments, the first heavy chain and the second heavy chain each comprise at least one amino acid modification that promotes heterodimerization of the first heavy chain with the second heavy chain. In some embodiments, the CH3 domain of the first heavy chain and the CH3 domain of the second heavy chain each comprise at least one amino acid modification that promotes heterodimerization of the first heavy chain with the second heavy chain.

In some embodiments, the hIL-12 protein is a schIL-12 protein (e.g., a schIL-12 protein described herein), wherein the C-terminus of the CH3 region of the first or second Ig heavy chain of the full-length antibody is operably connected to the N-terminus of the hscIL-12 polypeptide. In some embodiments, the hIL-12 protein is a schIL-12 protein (e.g., a schIL-12 protein described herein), wherein the C-terminus of the CH3 region of the first or second Ig heavy chain of the full-length antibody is directly operably connected to the N-terminus of the hscIL-12 polypeptide through a peptide bond (see, e.g., FIG. 2). In some embodiments, the hIL-12 protein is a schIL-12 protein (e.g., a schIL-12 protein described herein), wherein the C-terminus of the CH3 region of the first or second Ig heavy chain of the full-length antibody is indirectly operably connected to the N-terminus of the hscIL-12 polypeptide through a peptide linker. In some embodiments, the hIL-12 protein is a schIL-12 protein (e.g., a schIL-12 protein described herein), wherein the C-terminus of the CH3 region of the first or second Ig heavy chain of the full-length antibody is directly operably connected to the N-terminus of a peptide linker, the C-terminus of the peptide linker is directly operably connected to the N-terminus of the hscIL-12 polypeptide. Exemplary peptide linkers are described herein, see, e.g., § 5.3.4, Table 18, SEQ ID NOS: 66-81, 288-303, or 369.

In some embodiments, the full-length antibody (e.g., an anti-hCAIX full-length antibody) comprises a) a first polypeptide that comprises from N- to C-terminus a first light chain variable region (VL) and a first light chain constant region (CL); a second polypeptide that comprises from N- to C-terminus a first heavy chain variable region (VH), a first heavy chain constant region (CH), and A scIL-12; a third polypeptide that comprises from N- to C-terminus a second VH and a second CH; and a fourth polypeptide that comprises from N to C terminus a second VL, and a second CL. In some embodiments, the first CH region comprises from N to C terminus a CH1 domain, a hinge domain, a CH2 domain, and a CH3 domain. In some embodiments, the second CH region comprises from N- to C-terminus a CH1 domain, a hinge domain, a CH2 domain, and a CH3 domain. In some embodiments, the first polypeptide and the second polypeptide are connected via at least one disulfide bond. In some embodiments, the second polypeptide and the third polypeptide are connected via at least one disulfide bond. In some embodiments, the third polypeptide and the fourth polypeptide are connected via at least one disulfide bond. In some embodiments, the first CH and the second CH each comprise an amino acid modification that promotes heterodimerization of the first CH and the second CH.

### 5.3.6.2 Exemplary Fc Fusions Proteins

In some embodiments, the fusion protein comprises a first Fc region and a second Fc region, wherein the first and second Fc regions associate to form a dimer. In some embodiments, the N-terminus of the hIL-12p35 polypeptide of the hIL-12 protein is operably connected to the C-terminus of the CH3 region of the first Fc region and the N-terminus of the hIL-12p40 polypeptide of the hIL-12 protein is operably connected to the C-terminus of the CH3 region of the second Fc region. In some embodiments, the N-terminus of the hIL-12p35 polypeptide of the hIL-12 protein is directly operably connected to the C-terminus of the CH3 region of the first Fc region through a peptide bond and the N-terminus of the hIL-12p40 polypeptide of the hIL-12 protein is directly operably connected to the C-terminus of the CH3 region of the second Fc region through a peptide bond. In some embodiments, the N-terminus of the hIL-12p35 polypeptide of the hIL-12 protein is indirectly operably connected to the C-terminus of the CH3 region of the first Fc region through a peptide linker and the N-terminus of the hIL-12p40 polypeptide of the hIL-12 protein is indirectly operably connected to the C-terminus of the CH3 region of the second Fc region through a peptide linker. In some embodiments, (a) the C-terminus of the CH3 region of the first Fc region is operably connected to the N-terminus of a peptide linker, the C-terminus of the peptide linker is operably connected to the N-terminus of the hIL-12p35 polypeptide; and (b) the C-terminus of the CH3 region of the second Fc region is operably connected to the N-terminus of a peptide linker, the C-terminus of the peptide linker is operably connected to the N-terminus of the hIL-12p35 polypeptide. Exemplary peptide linkers are described herein, see, e.g., § 5.3.4, Table 18, SEQ ID NOS: 66-81, 288-303, or 369.

In some embodiments, the hIL-12 protein is a schIL-12 protein (e.g., a schIL-12 protein described herein), wherein the C-terminus of the CH3 region of the first or second Fc region is operably connected to the N-terminus of the hscIL-12 polypeptide. In some embodiments, the hIL-12 protein is a schIL-12 protein (e.g., a schIL-12 protein described herein), wherein the C-terminus of the CH3 region of the first or second Fc region is directly operably connected to the N-terminus of the hscIL-12 polypeptide through a peptide bond. In some embodiments, the hIL-12 protein is a schIL-12 protein (e.g., a schIL-12 protein described herein), wherein the C-terminus of the CH3 region of the first or second Fc region is indirectly operably connected to the N-terminus of the hscIL-12 polypeptide through a peptide linker. In some embodiments, the hIL-12 protein is a schIL-12 protein (e.g., a schIL-12 protein described herein), wherein the C-terminus of the CH3 region of the first or second Fc region is directly operably connected to the N-terminus of a peptide linker, the C-terminus of the peptide linker is directly operably connected to the N-terminus of the hscIL-12 polypeptide. Exemplary peptide linkers are described herein, see, e.g., § 5.3.4, Table 18, SEQ ID NOS: 66-81, 288-303, or 369.

In some embodiments, the C-terminus of the hIL-12p35 polypeptide of the hIL-12 protein is operably connected to the N-terminus of the first Fc region and the C-terminus of the hIL-12p40 polypeptide of the hIL-12 protein is operably connected to the N-terminus of the second Fc region. In some embodiments, the C-terminus of the hIL-12p35 polypeptide of the hIL-12 protein is directly operably connected to the N-terminus of the first Fc region through a peptide bond and the C-terminus of the hIL-12p40 polypeptide of the hIL-12 protein is directly operably connected to the N-terminus of the second Fc region through a peptide bond. In some embodiments, the C-terminus of the hIL-12p35 polypeptide of the hIL-12 protein is indirectly operably connected to the N-terminus of the first Fc region through a peptide linker and the C-terminus of the hIL-12p40 polypeptide of the hIL-12 protein is indirectly operably connected to the N-terminus of the second Fc region through a peptide linker. In some embodiments, (a) the N-terminus of the first Fc region is operably connected to the C-terminus of a peptide linker, the N-terminus of the peptide linker is operably connected to the C-terminus of the hIL-12p35 polypeptide; and (b) the N-terminus of the second Fc region is operably connected to the C-terminus of a peptide linker, the N-terminus of the peptide linker is operably connected to the C-terminus of the hIL-12p35 polypeptide. Exemplary peptide linkers are described herein, see, e.g., § 5.3.4, Table 18, SEQ ID NOS: 66-81, 288-303, or 369.

In some embodiments, the hIL-12 protein is a schIL-12 protein (e.g., a schIL-12 protein described herein), wherein the N-terminus of the first or second Fc region is operably connected to the C-terminus of the hscIL-12 polypeptide. In some embodiments, the hIL-12 protein is a schIL-12 protein (e.g., a schIL-12 protein described herein), wherein the N-terminus of the first or second Fc region is directly operably connected to the C-terminus of the hscIL-12 polypeptide through a peptide bond. In some embodiments, the hIL-12 protein is a schIL-12 protein (e.g., a schIL-12 protein described herein), wherein the N-terminus of the first or second Fc region is indirectly operably connected to the C-terminus of the hscIL-12 polypeptide through a peptide linker. In some embodiments, the hIL-12 protein is a schIL-12 protein (e.g., a schIL-12 protein described herein), wherein the N-terminus of the first or second Fc region is directly operably connected to the C-terminus of a peptide linker, the C-terminus of the peptide linker is directly operably connected to the C-terminus of the hscIL-12 polypeptide. Exemplary peptide linkers are described herein, see, e.g., § 5.3.4, Table 18, SEQ ID NOS: 66-81, 288-303, or 369.

### 5.3.6.3 Exemplary ScFv-Fc Fusion Proteins

In some embodiments, the fusion protein comprises a first scFv operably connected to the N-terminus of a first Fc region; and a second scFv operably connected to the N-terminus of a second Fc region; and wherein hIL-12p35 is operably connected to the C-terminus the first Fc region; and IL-12p40 is operably connected to the C-terminus of a second Fc region. See for example, FIG. 3.

In some embodiments, the fusion protein comprises a first polypeptide that comprises from N- to C-terminus a first scFv, a first Fc region, an optional first peptide linker, and IL-12p35; and a second polypeptide comprising from N- to C-terminus a second scFv, a second Fc region, an optional second peptide linker, and IL-12p40. In some embodiments, the first polypeptide and the second polypeptide are connected via at least one disulfide bond. In some embodiments, the first Fc region and the second Fc region each comprise an amino acid modification that promotes heterodimerization of the first Fc region and the second Fc region.

In some embodiments, the fusion protein comprises a first polypeptide that comprises from N- to C-terminus a first scFv, a first peptide linker, a first Fc region, a second peptide linker, and IL-12p35; and a second polypeptide comprising from N- to C-terminus a second scFv, a third peptide linker, a second Fc region, a fourth peptide linker, and IL-12p40. In some embodiments, the first polypeptide and the second polypeptide are connected via at least one disulfide bond. In some embodiments, the first Fc region and the second Fc region each comprise an amino acid modification that promotes heterodimerization of the first Fc region and the second Fc region.

### 5.3.6.4 Exemplary Dart Fc Fusion Proteins

In some embodiments, the fusion protein comprises a first polypeptide of a DART (i.e., a polypeptide comprising a first VH and VL of a DART) operably connected to the N-terminus of a first Fc region; and a second polypeptide of the DART (e.g., a polypeptide comprising the second VH and VL of the DART) operably connected to the N-terminus of a second Fc region; wherein hIL-12p35 is operably connected to the C-terminus a first Fc region; and IL-12p40 is operably connected to the C-terminus of a second Fc region. See for example, FIG. 6.

In some embodiments, the fusion protein comprises a first polypeptide that comprises from N- to C-terminus a first VL and a first VH of a DART, a first Fc region, and IL-12p35; and a second polypeptide comprising from N- to C-terminus a second VL and a second VH of the DART, a second Fc region, and IL-12p40; wherein first VL associates with the second VH to form a first antigen binding domain, and the second VL associates with the first VH to form a second antigen binding domain. In some embodiments, the first polypeptide and the second polypeptide are connected via at least one disulfide bond. In some embodiments, the first Fc region and the second Fc region each comprise an amino acid modification that promotes heterodimerization of the first Fc region and the second Fc region.

In some embodiments, the fusion protein comprises a first polypeptide that comprises from N- to C-terminus a first VH and a first VL of a DART, a first Fc region, and IL-12p35; and a second polypeptide comprising from N- to C-terminus a second VH and a second VL of the DART, a second Fc region, and IL-12p40; wherein first VL associates with the second VH to form a first antigen binding domain, and the second VL associates with the first VH to form a second antigen binding domain. In some embodiments, the first polypeptide and the second polypeptide are connected via at least one disulfide bond. In some embodiments, the first Fc region and the second Fc region each comprise an amino acid modification that promotes heterodimerization of the first Fc region and the second Fc region.

5.3.6.5 Exemplary Tandem ScFv scIL-12 Fc Fusion Proteins

In some embodiments, the fusion protein comprises two scFv antigen binding domains operably connected in tandem, and further operably connected to the N-terminus of a first Fc region; and a scIL-12 operably connected to the N-terminus of a second Fc region. See for example, FIG. 7.

In some embodiments, the fusion protein comprises a first polypeptide that comprises from N- to C-terminus a first scFv, a second scFv, and a first Fc region; and a second polypeptide comprising from N- to C-terminus scIL-12, an optional peptide linker, and a second Fc region. In some embodiments, the first polypeptide and the second polypeptide are connected via at least one disulfide bond. In some embodiments, the first Fc region and the second Fc region each comprise an amino acid modification that promotes heterodimerization of the first Fc region and the second Fc region.

In some embodiments, the fusion protein comprises a first polypeptide that comprises from N- to C-terminus a first scFv, a first peptide linker, a second scFv, a second peptide linker, and a first Fc region; and a second polypeptide comprising from N- to C-terminus scIL-12, an optional third linker, and a second Fc region. In some embodiments, the first polypeptide and the second polypeptide are connected via at least one disulfide bond. In some embodiments, the first Fc region and the second Fc region each comprise an amino acid modification that promotes heterodimerization of the first Fc region and the second Fc region.

5.3.6.6 Exemplary Tandem ScFv sdAb scIL-12 Fc Fusion Proteins

In some embodiments, the fusion protein comprises two scFv antigen binding domains operably connected in tandem, and further operably connected to the N-terminus of a first Fc region; and a first single domain antibody (sdAb) operably connected to a second Fc region, and a scIL-12 operably connected to the N-terminus of the sdAb. See for example, FIG. 8.

In some embodiments, the fusion protein comprises a first polypeptide that comprises from N- to C-terminus a first scFv, a second scFv, and a first Fc region; and a second polypeptide that comprises from N- to C-terminus scIL-12, an optional first peptide linker, a single domain antibody, an optional second peptide linker, and a second Fc region. In some embodiments, the first polypeptide and the second polypeptide are connected via at least one disulfide bond. In some embodiments, the first Fc region and the second Fc region each comprise an amino acid modification that promotes heterodimerization of the first Fc region and the second Fc region.

In some embodiments, the fusion protein comprises a first polypeptide that comprises from N- to C-terminus a first scFv, a first peptide linker, a second scFv, a second peptide linker, and a first Fc region; and a second polypeptide that comprises from N- to C-terminus scIL-12, a third peptide linker, a single domain antibody, a fourth peptide linker, and a second Fc region. In some embodiments, the first polypeptide and the second polypeptide are connected via at least one disulfide bond. In some embodiments, the first Fc region and the second Fc region each comprise an amino acid modification that promotes heterodimerization of the first Fc region and the second Fc region.

### 5.3.7 Exemplary Fusion Proteins & Polypeptides

The amino acid sequence of exemplary anti-hCAIX hIL-12 fusion polypeptides & proteins described herein is provided in Table 20. The anti-hCAIX hIL-12 fusion polypeptides and proteins provided in Table 20 are exemplary only, and not intended to be limiting.

The amino acid sequence of light chain and heavy chain regions common to one or more of the exemplary anti-hCAIX hIL-12 fusion proteins provided in Table 20, are provided in Table 19.

TABLE 19
The Amino Acid Sequence of Light Chain and Heavy Chain Regions Common to
One or More Exemplary Anti-hCAIX hIL-12 Fusion Proteins & Polypeptide

| | SEQ | |
|---|---|---|
| Description | ID NO | Amino Acid Sequence |
| Heavy | 304 | EVRLVESGGGLVKPGGSLRLSCAASGFTFSNYYMSWIRQAPEKRLELVSAIN |
| Chain- | | SDGGITYYLDTVKGRFTISRDNAKNSLYLQMNSLRAEDTALFYCARHRSGYF |
| Knob | | SMDYWGQGTSVTVSSASTKGPSVFPLAPSSKSTSGGTAALGCLVKDYFPEPV |
| IL-12p40 | | TVSWNSGALTSGVHTFPAVLQSSGLYSLSSVVTVPSSSLGTQTYICNVNHKP |
| | | SNTKVDKKVEPKSCDKTHTCPPCPAPELLGGPSVFLFPPKPKDTLMISRTPE |
| | | VTCVVVDVSHEDPEVKFNWYVDGVEVHNAKTKPREEQYNSTYRVVSVLTVLH |
| | | QDWLNGKEYKCKVSNKALPAPIEKTISKAKGQPREPQVYTLPPSRDELTKNQ |
| | | VSLWCLVKGFYPSDIAVEWESNGQPENNYKTTPPVLDSDGSFFLYSKLTVDK |
| | | SRWQQGNVFSCSVMHEALHNHYTQKSLSLSPGKGGGGSGGGGSGGGGSIWEL |
| | | KKDVYVVELDWYPDAPGEMVVLTCDTPEEDGITWTLDQSSEVLGSGKTLTIQ |
| | | VKEFGDAGQYTCHKGGEVLSHSLLLLHKKEDGIWSTDILKDQKEPKNKTFLR |
| | | CEAKNYSGRFTCWWLTTISTDLTFSVKSSRGSSDPQGVTCGAATLSAERVRG |
| | | DNKEYEYSVECQEDSACPAAEESLPIEVMVDAVHKLKYENYTSSFFIRDIIK |
| | | PDPPKNLQLKPLKNSRQVEVSWEYPDTWSTPHSYFSLTFCVQVQGKSKREKK |
| | | DRVFTDKTSATVICRKNASISVRAQDRYYSSSWSEWASVPCS |
| Heavy | 305 | EVRLVESGGGLVKPGGSLRLSCAASGFTFSNYYMSWIRQAPEKRLELVSAIN |
| Chain- | | SDGGITYYLDTVKGRFTISRDNAKNSLYLQMNSLRAEDTALFYCARHRSGYF |
| Hole | | SMDYWGQGTSVTVSSASTKGPSVFPLAPSSKSTSGGTAALGCLVKDYFPEPV |
| IL-12p35 | | TVSWNSGALTSGVHTFPAVLQSSGLYSLSSVVTVPSSSLGTQTYICNVNHKP |
| | | SNTKVDKKVEPKSCDKTHTCPPCPAPELLGGPSVFLFPPKPKDTLMISRTPE |
| | | VTCVVVDVSHEDPEVKFNWYVDGVEVHNAKTKPREEQYNSTYRVVSVLTVLH |
| | | QDWLNGKEYKCKVSNKALPAPIEKTISKAKGQPREPQVYTLPPSRDELTKNQ |
| | | VSLSCAVKGFYPSDIAVEWESNGQPENNYKTTPPVLDSDGSFFLVSKLTVDK |
| | | SRWQQGNVFSCSVMHEALHNHYTQKSLSLSPGKGGGGSGGGGSGGGGSRNLP |
| | | VATPDPGMFPCLHHSQNLLRAVSNMLQKARQTLEFYPCTSEEIDHEDITKDK |
| | | TSTVEACLPLELTKNESCLNSRETSFITNGSCLASRKTSEMMALCLSSIYED |
| | | LKMYQVEFKTMNAKLLMDPKRQIFLDQNMLAVIDELMQALNENSETVPQKSS |
| | | LEEPDFYKTKIKLCILLHAFRIRAVTIDRVMSYLNAS |
| Heavy | 306 | EVRLVESGGGLVKPGGSLRLSCAASGFTFSNYYMSWIRQAPEKRLELVSAIN |
| Chain- | | SDGGITYYLDTVKGRFTISRDNAKNSLYLQMNSLRAEDTALFYCARHRSGYF |
| Hole | | SMDYWGQGTSVTVSSASTKGPSVFPLAPSSKSTSGGTAALGCLVKDYFPEPV |
| | | TVSWNSGALTSGVHTFPAVLQSSGLYSLSSVVTVPSSSLGTQTYICNVNHKP |
| | | SNTKVDKKVEPKSCDKTHTCPPCPAPELLGGPSVFLFPPKPKDTLMISRTPE |
| | | VTCVVVDVSHEDPEVKFNWYVDGVEVHNAKTKPREEQYNSTYRVVSVLTVLH |
| | | QDWLNGKEYKCKVSNKALPAPIEKTISKAKGQPREPQVYTLPPSRDELTKNQ |
| | | VSLSCAVKGFYPSDIAVEWESNGQPENNYKTTPPVLDSDGSFFLVSKLTVDK |
| | | SRWQQGNVFSCSVMHEALHNHYTQKSLSLSPGK |
| Light Chain | 245 | EIVMTQSPATLSVSPGERATLSCKASQNVVSAVAWYQQKPGQSPRLLIYSAS |
| | | NRYTGIPARFSGSGSGTEFTLTISSLQSEDFAAYYCQQYSNYPWTFGGGTKV |
| | | EIKRTVAAPSVFIFPPSDEQLKSGTASVVCLLNNFYPREAKVQWKVDNALQS |
| | | GNSQESVTEQDSKDSTYSLSSTLTLSKADYEKHKVYACEVTHQGLSSPVTKS |
| | | FNRGEC |

As described above, the amino acid sequence of exemplary anti-hCAIX hIL-12 fusion polypeptides & proteins described herein is provided in Table 20. The anti-hCAIX hIL-12 fusion polypeptides and proteins provided in Table 20 are exemplary only, and not intended to be limiting.

TABLE 20
The Amino Acid Sequence of Exemplary Anti-hCAIX hIL-12 Fusion Proteins &
Polypeptides

| | | SEQ | |
|---|---|---|---|
| Description | | ID NO | Amino Acid Sequence |
| BCA316 | Heavy Chain | 304 | See Table 19. |
| (BCA351) | (HC) (Knob) | | |
| See e.g., | IL-12p40 | | |
| FIG. 1 | HC (Hole) | 305 | EVRLVESGGGLVKPGGSLRLSCAASGFTFSNYYMSWIRQAPE |
| | IL-12p35 | | KRLELVSAINSDGGITYYLDTVKGRFTISRDNAKNSLYLQMN |
| | | | SLRAEDTALFYCARHRSGYFSMDYWGQGTSVTVSSASTKGPS |
| | | | VFPLAPSSKSTSGGTAALGCLVKDYFPEPVTVSWNSGALTSG |
| | | | VHTFPAVLQSSGLYSLSSVVTVPSSSLGTQTYICNVNHKPSN |
| | | | TKVDKKVEPKSCDKTHTCPPCPAPELLGGPSVFLFPPKPKDT |
| | | | LMISRTPEVTCVVVDVSHEDPEVKFNWYVDGVEVHNAKTKPR |
| | | | EEQYNSTYRVVSVLTVLHQDWLNGKEYKCKVSNKALPAPIEK |
| | | | TISKAKGQPREPQVYTLPPSRDELTKNQVSLSCAVKGFYPSD |
| | | | IAVEWESNGQPENNYKTTPPVLDSDGSFFLVSKLTVDKSRWQ |
| | | | QGNVFSCSVMHEALHNHYTQKSLSLSPGKGGGGSGGGGSGGG |
| | | | GSRNLPVATPDPGMFPCLHHSQNLLRAVSNMLQKARQTLEFY |
| | | | PCTSEEIDHEDITKDKTSTVEACLPLELTKNESCLNSRETSE |
| | | | ITNGSCLASRKTSFMMALCLSSIYEDLKMYQVEFKTMNAKLL |
| | | | MDPKRQIFLDQNMLAVIDELMQALNENSETVPQKSSLEEPDE |
| | | | YKTKIKLCILLHAFRIRAVTIDRVMSYLNAS |
| | Light Chain | 245 | See Table 19. |
| | (LC) | | |
| BCA307.16 | Heavy Chain | 304 | See Table 19. |
| See e.g., | (Knob) | | |
| FIG. 1 | IL-12p40 | | |
| | Heavy Chain | 307 | EVRLVESGGGLVKPGGSLRLSCAASGFTFSNYYMSWIRQAPE |
| | (Hole) | | KRLELVSAINSDGGITYYLDTVKGRFTISRDNAKNSLYLQMN |
| | IL-12p35 | | SLRAEDTALFYCARHRSGYFSMDYWGQGTSVTVSSASTKGPS |
| | (variant A) | | VFPLAPSSKSTSGGTAALGCLVKDYFPEPVTVSWNSGALTSG |
| | | | VHTFPAVLQSSGLYSLSSVVTVPSSSLGTQTYICNVNHKPSN |
| | | | TKVDKKVEPKSCDKTHTCPPCPAPELLGGPSVFLFPPKPKDT |
| | | | LMISRTPEVTCVVVDVSHEDPEVKFNWYVDGVEVHNAKTKPR |
| | | | EEQYNSTYRVVSVLTVLHQDWLNGKEYKCKVSNKALPAPIEK |
| | | | TISKAKGQPREPQVYTLPPSRDELTKNQVSLSCAVKGFYPSD |
| | | | IAVEWESNGQPENNYKTTPPVLDSDGSFFLVSKLTVDKSRWQ |
| | | | QGNVFSCSVMHEALHNHYTQKSLSLSPGKGGGGSGGGGSGGG |
| | | | GSRNLPVATPDPGMFPCLHHSQNLLRAVSNMLQKNESCLNSR |
| | | | ETSFITNGSCLASRKTSEMMALCLSSIYEDLKMYQVEFKTMN |
| | | | AKLLMDPKRQIFLDQNMLAVIDELMQALNENSETVPQKSSLE |
| | | | EPDFYKTKIKLCILLHAFRIRAVTIDRVMSYLNAS |
| | Light Chain | 245 | See Table 19. |
| BCA308.16 | Heavy Chain | 304 | See Table 19. |
| See e.g., | (Knob) | | |
| FIG. 1 | IL-12p40 | | |
| | Heavy Chain | 308 | EVRLVESGGGLVKPGGSLRLSCAASGFTFSNYYMSWIRQAPE |
| | (Hole) | | KRLELVSAINSDGGITYYLDTVKGRFTISRDNAKNSLYLQMN |
| | IL-12p35 | | SLRAEDTALFYCARHRSGYFSMDYWGQGTSVTVSSASTKGPS |
| | (variant B) | | VFPLAPSSKSTSGGTAALGCLVKDYFPEPVTVSWNSGALTSG |
| | | | VHTFPAVLQSSGLYSLSSVVTVPSSSLGTQTYICNVNHKPSN |
| | | | TKVDKKVEPKSCDKTHTCPPCPAPELLGGPSVFLFPPKPKDT |
| | | | LMISRTPEVTCVVVDVSHEDPEVKFNWYVDGVEVHNAKTKPR |
| | | | EEQYNSTYRVVSVLTVLHQDWLNGKEYKCKVSNKALPAPIEK |
| | | | TISKAKGQPREPQVYTLPPSRDELTKNQVSLSCAVKGFYPSD |
| | | | IAVEWESNGQPENNYKTTPPVLDSDGSFFLVSKLTVDKSRWQ |
| | | | QGNVFSCSVMHEALHNHYTQKSLSLSPGKGGGGSGGGGSGGG |
| | | | GSRNLPVATPDPGMFPCLHHSQNLLRAVSNMLQKARQTLKHY |
| | | | SCTSEEIDHEDITKDKTSTVEACLPLELTKNESCLNSRETSE |
| | | | ITNGSCLASRKTSEMMALCLSSIYEDLKMYQVEFKTMNAKLL |
| | | | MDPHRQIFLDQNMLAVIDELMQALNENSETVPQKSSLEEPDP |
| | | | YKTKIKLCILLHAFRIRAVTIDRVMSYLNAS |
| | Light Chain | 245 | See Table 19. |
| BCA323.16 | Heavy Chain | 304 | See Table 19. |
| See e.g., | (Knob) | | |
| FIG. 1 | IL-12p40 | | |
| | Heavy Chain | 309 | EVRLVESGGGLVKPGGSLRLSCAASGFTFSNYYMSWIRQAPE |
| | (Hole) | | KRLELVSAINSDGGITYYLDTVKGRFTISRDNAKNSLYLQMN |
| | IL-12p35 | | SLRAEDTALFYCARHRSGYFSMDYWGQGTSVTVSSASTKGPS |
| | (variant C) | | VFPLAPSSKSTSGGTAALGCLVKDYFPEPVTVSWNSGALTSG |
| | | | VHTFPAVLQSSGLYSLSSVVTVPSSSLGTQTYICNVNHKPSN |
| | | | TKVDKKVEPKSCDKTHTCPPCPAPELLGGPSVFLFPPKPKDT |
| | | | LMISRTPEVTCVVVDVSHEDPEVKFNWYVDGVEVHNAKTKPR |
| | | | EEQYNSTYRVVSVLTVLHQDWLNGKEYKCKVSNKALPAPIEK |
| | | | TISKAKGQPREPQVYTLPPSRDELTKNQVSLSCAVKGFYPSD |
| | | | IAVEWESNGQPENNYKTTPPVLDSDGSFFLVSKLTVDKSRWQ |
| | | | QGNVFSCSVMHEALHNHYTQKSLSLSPGKGGGGSGGGGSGGG |
| | | | GSRNLPVATPDPGMFPCLHHSQNLLRAVSNMLQKARQTLEFY |
| | | | PCTSEEIDHEDITKDKTSTVEACLPLELTKNESCLNSRETSE |
| | | | ITNGSCLASRKTSFMMALCLSSIYEDLKMYQVEFKTMNAKLL |
| | | | MDPKRQIFLDQNMLAVIDELMQALNENSETVPQKSSLEEPDA |
| | | | AKTKIKLCILLHAFRIRAVTIDRVMSYLNAS |
| | Light Chain | 245 | See Table 19. |
| BCA324.16 | Heavy Chain | 304 | See Table 19. |
| See e.g., | (Knob) | | |
| FIG. 1 | IL-12p40 | | |
| | Heavy Chain | 310 | EVRLVESGGGLVKPGGSLRLSCAASGFTFSNYYMSWIRQAPE |
| | (Hole) | | KRLELVSAINSDGGITYYLDTVKGRFTISRDNAKNSLYLQMN |
| | IL-12p35 | | SLRAEDTALFYCARHRSGYFSMDYWGQGTSVTVSSASTKGPS |
| | (variant D) | | VFPLAPSSKSTSGGTAALGCLVKDYFPEPVTVSWNSGALTSG |
| | | | VHTFPAVLQSSGLYSLSSVVTVPSSSLGTQTYICNVNHKPSN |
| | | | TKVDKKVEPKSCDKTHTCPPCPAPELLGGPSVFLFPPKPKDT |
| | | | LMISRTPEVTCVVVDVSHEDPEVKFNWYVDGVEVHNAKTKPR |
| | | | EEQYNSTYRVVSVLTVLHQDWLNGKEYKCKVSNKALPAPIEK |
| | | | TISKAKGQPREPQVYTLPPSRDELTKNQVSLSCAVKGFYPSD |
| | | | IAVEWESNGQPENNYKTTPPVLDSDGSFFLVSKLTVDKSRWQ |
| | | | QGNVFSCSVMHEALHNHYTQKSLSLSPGKGGGGSGGGGSGGG |
| | | | GSRNLPVATPDPGMFPCLHHSQNLLRAVSNMLQKARQTLEFY |
| | | | PCTSEEIDHEDITKDKTSTVEACLPLELTKNESCLNSRETSE |
| | | | ITNGSCLASRKTSFMMALCLSSIYEDLKMYQVEFKTMNAKLL |
| | | | MDPKRQIFLDQNMLAVIDELMQALNENSETVPQKSSLEEPDA |
| | | | YKTKIKLCILLHAFRIRAVTIDRVMSYLNAS |
| | Light Chain | 245 | See Table 19. |
| BCA325.16 | Heavy Chain | 304 | See Table 19. |
| See e.g., | (Knob) | | |
| FIG. 1 | IL-12p40 | | |
| | Heavy Chain | 311 | EVRLVESGGGLVKPGGSLRLSCAASGFTFSNYYMSWIRQAPE |
| | (Hole) | | KRLELVSAINSDGGITYYLDTVKGRFTISRDNAKNSLYLQMN |
| | IL-12p35 | | SLRAEDTALFYCARHRSGYFSMDYWGQGTSVTVSSASTKGPS |
| | (variant E) | | VFPLAPSSKSTSGGTAALGCLVKDYFPEPVTVSWNSGALTSG |
| | | | VHTFPAVLQSSGLYSLSSVVTVPSSSLGTQTYICNVNHKPSN |
| | | | TKVDKKVEPKSCDKTHTCPPCPAPELLGGPSVFLFPPKPKDT |
| | | | LMISRTPEVTCVVVDVSHEDPEVKFNWYVDGVEVHNAKTKPR |
| | | | EEQYNSTYRVVSVLTVLHQDWLNGKEYKCKVSNKALPAPIEK |
| | | | TISKAKGQPREPQVYTLPPSRDELTKNQVSLSCAVKGFYPSD |
| | | | IAVEWESNGQPENNYKTTPPVLDSDGSFFLVSKLTVDKSRWQ |
| | | | QGNVFSCSVMHEALHNHYTQKSLSLSPGKGGGGSGGGGSGGG |
| | | | GSRNLPVATPDPGMFPCLHHSQNLLRAVSNMLQKARQTLEFY |
| | | | PCTSEEIDHEDITKDKTSTVEACLPLELTKNESCLNSRETSF |
| | | | ITNGSCLASRKTSEMMALCLSSIYEDLKMYQVEFKTMNAKLL |
| | | | MDPKRQIFLDQNMLAVIDELMQALNENSETVPQKSSLEEPDE |
| | | | AKTKIKLCILLHAFRIRAVTIDRVMSYLNAS |
| | Light Chain | 245 | See Table 19. |
| BCA309.16 | Heavy Chain | 312 | EVRLVESGGGLVKPGGSLRLSCAASGFTFSNYYMSWIRQAPE |
| See e.g., | (Knob) | | KRLELVSAINSDGGITYYLDTVKGRFTISRDNAKNSLYLQMN |
| FIG. 1 | IL-12p40 | | SLRAEDTALFYCARHRSGYFSMDYWGQGTSVTVSSASTKGPS |
| | (variant A) | | VFPLAPSSKSTSGGTAALGCLVKDYFPEPVTVSWNSGALTSG |
| | | | VHTFPAVLQSSGLYSLSSVVTVPSSSLGTQTYICNVNHKPSN |
| | | | TKVDKKVEPKSCDKTHTCPPCPAPELLGGPSVFLFPPKPKDT |
| | | | LMISRTPEVTCVVVDVSHEDPEVKFNWYVDGVEVHNAKTKPR |
| | | | EEQYNSTYRVVSVLTVLHQDWLNGKEYKCKVSNKALPAPIEK |
| | | | TISKAKGQPREPQVYTLPPSRDELTKNQVSLWCLVKGFYPSD |
| | | | IAVEWESNGQPENNYKTTPPVLDSDGSFFLYSKLTVDKSRWQ |
| | | | QGNVFSCSVMHEALHNHYTQKSLSLSPGKGGGGSGGGGSGGG |
| | | | GSIWELKKDVYVVELDWYPDAPGEMVVLTCDTPEEDGITWTL |
| | | | DQSSEVLGSGKTLTIQVKEFGDAGQYTCHKGGEVLSHSLLLL |
| | | | HKKEDGIWSTDILKDQKEPKNKTFLRCEAKNYSGRFTCWWLT |
| | | | TISTDLTFSVKSSRGSSDPQGVTCGAATLSAERVRGDNKEYE |
| | | | YSVECQEDSACPAAEESLPIEVMVDAVHKLKYENYTSSFFIR |
| | | | DIIKPDPPKNLQLKPLKNSRQVEVSWEYPDTWSTPHSYESLT |
| | | | FCVQVQGASAAEAADAVETDKTSATVICRKNASISVRAQDRY |
| | | | YSSSWSEWASVPCS |
| | Heavy Chain | 305 | See Table 19. |
| | (Hole) | | |
| | IL-12p35 | | |
| | Light Chain | 245 | See Table 19. |
| BCA310.16: | Heavy Chain | 313 | EVRLVESGGGLVKPGGSLRLSCAASGFTFSNYYMSWIRQAPE |
| See e.g., | (Knob) | | KRLELVSAINSDGGITYYLDTVKGRFTISRDNAKNSLYLQMN |
| FIG. 1 | IL-12p40 | | SLRAEDTALFYCARHRSGYFSMDYWGQGTSVTVSSASTKGPS |
| | (variant B) | | VFPLAPSSKSTSGGTAALGCLVKDYFPEPVTVSWNSGALTSG |
| | | | VHTFPAVLQSSGLYSLSSVVTVPSSSLGTQTYICNVNHKPSN |
| | | | TKVDKKVEPKSCDKTHTCPPCPAPELLGGPSVFLFPPKPKDT |
| | | | LMISRTPEVTCVVVDVSHEDPEVKFNWYVDGVEVHNAKTKPR |
| | | | EEQYNSTYRVVSVLTVLHQDWLNGKEYKCKVSNKALPAPIEK |
| | | | TISKAKGQPREPQVYTLPPSRDELTKNQVSLWCLVKGFYPSD |
| | | | IAVEWESNGQPENNYKTTPPVLDSDGSFFLYSKLTVDKSRWQ |
| | | | QGNVFSCSVMHEALHNHYTQKSLSLSPGKGGGGSGGGGSGGG |
| | | | GSFLRCEAKNYSGRFTCWWLTTISTDLTFSVKSSRGSSDPQG |
| | | | VTCGAATLSAERVRGDNKEYEYSVECQEDSACPAAEESLPIE |
| | | | VMVDAVHKLKYENYTSSFFIRDIIKPDPPKNLQLKPLKNSRQ |
| | | | VEVSWEYPDTWSTPHSYFSLTFCVQVQGKSKREKKDRVFTDK |
| | | | TSATVICRKNASISVRAQDRYYSSSWSEWASVPCS |
| | Heavy Chain | 305 | See Table 19. |
| | (Hole) | | |
| | IL-12p35 | | |
| | Light Chain | 245 | See Table 19. |
| BCA311.16: | Heavy Chain | 314 | EVRLVESGGGLVKPGGSLRLSCAASGFTFSNYYMSWIRQAPE |
| See e.g., | (Knob) | | KRLELVSAINSDGGITYYLDTVKGRFTISRDNAKNSLYLQMN |
| FIG. 1 | IL-12p40 | | SLRAEDTALFYCARHRSGYFSMDYWGQGTSVTVSSASTKGPS |
| | (variant C) | | VFPLAPSSKSTSGGTAALGCLVKDYFPEPVTVSWNSGALTSG |
| | | | VHTFPAVLQSSGLYSLSSVVTVPSSSLGTQTYICNVNHKPSN |
| | | | TKVDKKVEPKSCDKTHTCPPCPAPELLGGPSVFLFPPKPKDT |
| | | | LMISRTPEVTCVVVDVSHEDPEVKFNWYVDGVEVHNAKTKPR |
| | | | EEQYNSTYRVVSVLTVLHQDWLNGKEYKCKVSNKALPAPIEK |
| | | | TISKAKGQPREPQVYTLPPSRDELTKNQVSLWCLVKGFYPSD |
| | | | IAVEWESNGQPENNYKTTPPVLDSDGSFFLYSKLTVDKSRWQ |
| | | | QGNVFSCSVMHEALHNHYTQKSLSLSPGKGGGGSGGGGSGGG |
| | | | GSIWELKKDVYVVELDWYPDAPGEMVVLTCDTPEEDGITWTL |
| | | | DQSSEVLGSGKTLTIQVKEFGDAGQYTCHKGGEVLSHSLLLL |
| | | | HKKEDGIWSTDILKDQKEPKNKTFLRCEAKNYSGRFTCWWLT |
| | | | TISTDLTFSVKSSRGSSDPQGVTCGAATLSAERVRGDNKEYE |
| | | | YSVECQEDSACPAAEEIES |
| | Heavy Chain | 305 | See Table 19. |
| | (Hole) | | |
| | IL-12p35 | | |
| | Light Chain | 245 | See Table 19. |
| BCA317: | Heavy Chain | 315 | EVRLVESGGGLVKPGGSLRLSCAASGFTFSNYYMSWIRQAPE |
| See e.g., | (Knob) | | KRLELVSAINSDGGITYYLDTVKGRFTISRDNAKNSLYLQMN |
| FIG. 1 | IL-12p40 | | SLRAEDTALFYCARHRSGYFSMDYWGQGTSVTVSSASTKGPS |
| | (variant D) | | VFPLAPSSKSTSGGTAALGCLVKDYFPEPVTVSWNSGALTSG |
| | | | VHTFPAVLQSSGLYSLSSVVTVPSSSLGTQTYICNVNHKPSN |
| | | | TKVDKKVEPKSCDKTHTCPPCPAPELLGGPSVFLFPPKPKDT |
| | | | LMISRTPEVTCVVVDVSHEDPEVKFNWYVDGVEVHNAKTKPR |
| | | | EEQYNSTYRVVSVLTVLHQDWLNGKEYKCKVSNKALPAPIEK |
| | | | TISKAKGQPREPQVYTLPPSRDELTKNQVSLWCLVKGFYPSD |
| | | | IAVEWESNGQPENNYKTTPPVLDSDGSFFLYSKLTVDKSRWQ |
| | | | QGNVFSCSVMHEALHNHYTQKSLSLSPGKGGGGSGGGGSGGG |
| | | | GSIWELKKDVYVVELDWYPDAPGEMVVLTCDTPEEDGITWTL |
| | | | DQSSEVLGSGKTLTIQVKKFGDAGQYTCHKGGEVLSHSLLLL |
| | | | HKKEDGIWSTDILKDQEEPKNKTFLRCEAKNYSGRFTCWWLT |
| | | | TISTDLTFSVKSSRGSSDPQGVTCGAATLSAERVRGDNKEYE |
| | | | YSVECQEDSACPAAEESLPIEVMVDAVHKLKYENYTSSFFIR |
| | | | DIIKPDPPKNLQLKPLKNSRQVEVSWEYPDTWSTPHSYFSLT |
| | | | FCVQVQGKSKREKEDRVFTDKTSATVICRKNASISVRAQDRY |
| | | | YSSSWSEWASVPCS |
| | Heavy Chain | 305 | See Table 19. |
| | (Hole) | | |
| | IL-12p35 | | |
| | Light Chain | 245 | See Table 19. |
| BCA318.16 | Heavy Chain | 316 | EVRLVESGGGLVKPGGSLRLSCAASGFTFSNYYMSWIRQAPE |
| See e.g., | (Knob) | | KRLELVSAINSDGGITYYLDTVKGRFTISRDNAKNSLYLQMN |
| FIG. 1 | IL-12p40 | | SLRAEDTALFYCARHRSGYFSMDYWGQGTSVTVSSASTKGPS |
| | (variant E) | | VFPLAPSSKSTSGGTAALGCLVKDYFPEPVTVSWNSGALTSG |
| | | | VHTFPAVLQSSGLYSLSSVVTVPSSSLGTQTYICNVNHKPSN |
| | | | TKVDKKVEPKSCDKTHTCPPCPAPELLGGPSVFLFPPKPKDT |
| | | | LMISRTPEVTCVVVDVSHEDPEVKFNWYVDGVEVHNAKTKPR |
| | | | EEQYNSTYRVVSVLTVLHQDWLNGKEYKCKVSNKALPAPIEK |
| | | | TISKAKGQPREPQVYTLPPSRDELTKNQVSLWCLVKGFYPSD |
| | | | IAVEWESNGQPENNYKTTPPVLDSDGSFFLYSKLTVDKSRWQ |
| | | | QGNVFSCSVMHEALHNHYTQKSLSLSPGKGGGGSGGGGSGGG |
| | | | GSIWELKKDVYVVELDWYPDAPGEMVVLTCDTPEEDGITWTL |
| | | | DQSSEVLGSGKTLTIQVKEFGDAGQYTCHKGGEVLSHSLLLL |
| | | | HKKEDGIWSTDILKDQKEPKNKTFLRCEAKNYSGRFTCWWLT |
| | | | TISTDLTFSVKSSRGSSDPQGVTCGAATLSAERVRGDNKEYE |
| | | | YSVECQEDSACPAAEEIESIKSMVDAVHKLKYENYTSSFFIR |
| | | | DIIKPDPPKNLQLKPLKNSRQVEVSWEYPDTWSTPHSYESLT |
| | | | FCVQVQGKSKREKKDRVFTDKTSATVICRKNASISVRAQDRY |
| | | | YSSSWSEWASVPCS |
| | Heavy Chain | 305 | See Table 19. |
| | (Hole) | | |
| | IL-12p35 | | |
| | Light Chain | 245 | See Table 19. |
| BCA319.16 | Heavy Chain | 317 | EVRLVESGGGLVKPGGSLRLSCAASGFTFSNYYMSWIRQAPE |
| See e.g., | (Knob) | | KRLELVSAINSDGGITYYLDTVKGRFTISRDNAKNSLYLQMN |
| FIG. 1 | IL-12p40 | | SLRAEDTALFYCARHRSGYFSMDYWGQGTSVTVSSASTKGPS |
| | (variant F) | | VFPLAPSSKSTSGGTAALGCLVKDYFPEPVTVSWNSGALTSG |
| | | | VHTFPAVLQSSGLYSLSSVVTVPSSSLGTQTYICNVNHKPSN |
| | | | TKVDKKVEPKSCDKTHTCPPCPAPELLGGPSVFLFPPKPKDT |
| | | | LMISRTPEVTCVVVDVSHEDPEVKFNWYVDGVEVHNAKTKPR |
| | | | EEQYNSTYRVVSVLTVLHQDWLNGKEYKCKVSNKALPAPIEK |
| | | | TISKAKGQPREPQVYTLPPSRDELTKNQVSLWCLVKGFYPSD |
| | | | IAVEWESNGQPENNYKTTPPVLDSDGSFFLYSKLTVDKSRWQ |
| | | | QGNVFSCSVMHEALHNHYTQKSLSLSPGKGGGGSGGGGSGGG |
| | | | GSIWELKKDVYVVELDWYPDAPGEMVVLTCDTPEEDGITWTL |
| | | | DQSSEVLGSGKTLTIQVKEFGDAGQYTCHKGGEVLSHSLLLL |
| | | | HKKEDGIWSTDILKDQKEPKNKTFLRCEAKNYSGRFTCWWLT |
| | | | TISTDLTFSVKSSRGSSDPQGVTCGAATLSAERVRGDNKEYE |
| | | | YSVECQEDSACPAAEEIVSIKIMVDAVHKLKYENYTSSFFIR |
| | | | DIIKPDPPKNLQLKPLKNSRQVEVSWEYPDTWSTPHSYFSLT |
| | | | FCVQVQGKSKREKKDRVFTDKTSATVICRKNASISVRAQDRY |
| | | | YSSSWSEWASVPCS |
| | Heavy Chain | 305 | See Table 19. |
| | (Hole) | | |
| | IL-12p35 | | |
| | Light Chain | 245 | See Table 19. |
| BCA320.16 | Heavy Chain | 318 | EVRLVESGGGLVKPGGSLRLSCAASGFTFSNYYMSWIRQAPE |
| See e.g., | (Knob) | | KRLELVSAINSDGGITYYLDTVKGRFTISRDNAKNSLYLQMN |
| FIG. 1 | IL-12p40 | | SLRAEDTALFYCARHRSGYFSMDYWGQGTSVTVSSASTKGPS |
| | (variant G) | | VFPLAPSSKSTSGGTAALGCLVKDYFPEPVTVSWNSGALTSG |
| | | | VHTFPAVLQSSGLYSLSSVVTVPSSSLGTQTYICNVNHKPSN |
| | | | TKVDKKVEPKSCDKTHTCPPCPAPELLGGPSVFLFPPKPKDT |
| | | | LMISRTPEVTCVVVDVSHEDPEVKFNWYVDGVEVHNAKTKPR |
| | | | EEQYNSTYRVVSVLTVLHQDWLNGKEYKCKVSNKALPAPIEK |
| | | | TISKAKGQPREPQVYTLPPSRDELTKNQVSLWCLVKGFYPSD |
| | | | IAVEWESNGQPENNYKTTPPVLDSDGSFFLYSKLTVDKSRWQ |
| | | | QGNVFSCSVMHEALHNHYTQKSLSLSPGKGGGGSGGGGSGGG |
| | | | GSIWELKKDVYVVELDWYPDAPGEMVVLTCDTPEEDGITWTL |
| | | | DQSSEVLGSGKTLTIQVKEFGDAGQYTCHKGGEVLSHSLLLL |
| | | | HKKEDGIWSTDILKDQKEPKNKTELRCEAKNYSGRFTCWWLT |
| | | | TISTDLTFSVKSSRGSSDPQGVTCGAATLSAERVRGDNKEYE |
| | | | YSVECQEDSACPAAEEIQSIKGMVDAVHKLKYENYTSSFFIR |
| | | | DIIKPDPPKNLQLKPLKNSRQVEVSWEYPDTWSTPHSYFSLT |
| | | | FCVQVQGKSKREKKDRVFTDKTSATVICRKNASISVRAQDRY |
| | | | YSSSWSEWASVPCS |
| | Heavy Chain | 305 | See Table 19. |
| | (Hole) | | |
| | IL-12p35 | | |
| | Light Chain | 245 | See Table 19. |
| BCA321.16 | Heavy Chain | 319 | EVRLVESGGGLVKPGGSLRLSCAASGFTFSNYYMSWIRQAPE |
| See e.g., | (Knob) | | KRLELVSAINSDGGITYYLDTVKGRFTISRDNAKNSLYLQMN |
| FIG. 1 | IL-12p40 | | SLRAEDTALFYCARHRSGYFSMDYWGQGTSVTVSSASTKGPS |
| | (variant H) | | VFPLAPSSKSTSGGTAALGCLVKDYFPEPVTVSWNSGALTSG |
| | | | VHTFPAVLQSSGLYSLSSVVTVPSSSLGTQTYICNVNHKPSN |
| | | | TKVDKKVEPKSCDKTHTCPPCPAPELLGGPSVFLFPPKPKDT |
| | | | LMISRTPEVTCVVVDVSHEDPEVKFNWYVDGVEVHNAKTKPR |
| | | | EEQYNSTYRVVSVLTVLHQDWLNGKEYKCKVSNKALPAPIEK |
| | | | TISKAKGQPREPQVYTLPPSRDELTKNQVSLWCLVKGFYPSD |
| | | | IAVEWESNGQPENNYKTTPPVLDSDGSFFLYSKLTVDKSRWQ |
| | | | QGNVFSCSVMHEALHNHYTQKSLSLSPGKGGGGSGGGGSGGG |
| | | | GSIWELKKDVYVVELDWYPDAPGEMVVLTCDTPEEDGITWTL |
| | | | DQSSEVLGSGKTLTIQVKEFGDAGQYTCHKGGEVLSHSLLLL |
| | | | HKKEDGIWSTDILKDQKEPKNKTFLRCEAKNYSGRFTCWWLT |
| | | | TISTDLTFSVKSSRGSSDPQGVTCGAATLSAERVRGDNKEYE |
| | | | YSVECQEDSACPAAEESLPIEVMVDAVHIKYENYTSSFFIRD |
| | | | IIKPDPPKNLQLKPLKNSRQVEVSWEYPDTWSTPHSYESLTF |
| | | | CVQVQGKSKREKKDRVFTDKTSATVICRKNASISVRAQDRYY |
| | | | SSSWSEWASVPCS |
| | Heavy Chain | 305 | See Table 19. |
| | (Hole) | | |
| | IL-12p35 | | |
| | Light Chain | 245 | See Table 19. |
| BCA322.16 | Heavy Chain | 320 | EVRLVESGGGLVKPGGSLRLSCAASGFTFSNYYMSWIRQAPE |
| See e.g., | (Knob) | | KRLELVSAINSDGGITYYLDTVKGRFTISRDNAKNSLYLQMN |
| FIG. 1 | IL-12p40 | | SLRAEDTALFYCARHRSGYFSMDYWGQGTSVTVSSASTKGPS |
| | (variant I) | | VFPLAPSSKSTSGGTAALGCLVKDYFPEPVTVSWNSGALTSG |
| | | | VHTFPAVLQSSGLYSLSSVVTVPSSSLGTQTYICNVNHKPSN |
| | | | TKVDKKVEPKSCDKTHTCPPCPAPELLGGPSVFLFPPKPKDT |
| | | | LMISRTPEVTCVVVDVSHEDPEVKFNWYVDGVEVHNAKTKPR |
| | | | EEQYNSTYRVVSVLTVLHQDWLNGKEYKCKVSNKALPAPIEK |
| | | | TISKAKGQPREPQVYTLPPSRDELTKNQVSLWCLVKGFYPSD |
| | | | IAVEWESNGQPENNYKTTPPVLDSDGSFFLYSKLTVDKSRWQ |
| | | | QGNVFSCSVMHEALHNHYTQKSLSLSPGKGGGGSGGGGSGGG |
| | | | GSIWELKKDVYVVELDWYPDAPGEMVVLTCDTPEEDGITWTL |
| | | | DQSSEVLGSGKTLTIQVKEFGDAGQYTCHKGGEVLSHSLLLL |
| | | | HKKEDGIWSTDILKDQKEPKNKTFLRCEAKNYSGRFTCWWLT |
| | | | TISTDLTFSVKSSRGSSDPQGVTCGAATLSAERVRGDNKEYE |
| | | | YSVECQEDSACPAAEESLPIEVMVDAVHSITSANYTSSFFIR |
| | | | DIIKPDPPKNLQLKPLKNSRQVEVSWEYPDTWSTPHSYESLT |
| | | | FCVQVQGKSKREKKDRVFTDKTSATVICRKNASISVRAQDRY |
| | | | YSSSWSEWASVPCS |
| | Heavy Chain | 305 | See Table 19. |
| | (Hole) | | |
| | IL-12p35 | | |
| | Light Chain | 245 | See Table 19. |
| BCA326.16 | Heavy Chain | 321 | EVRLVESGGGLVKPGGSLRLSCAASGFTFSNYYMSWIRQAPE |
| See e.g., | (Knob) | | KRLELVSAINSDGGITYYLDTVKGRFTISRDNAKNSLYLQMN |
| FIG. 1 | IL-12p40 | | SLRAEDTALFYCARHRSGYFSMDYWGQGTSVTVSSASTKGPS |
| | (variant J) | | VFPLAPSSKSTSGGTAALGCLVKDYFPEPVTVSWNSGALTSG |
| | | | VHTFPAVLQSSGLYSLSSVVTVPSSSLGTQTYICNVNHKPSN |
| | | | TKVDKKVEPKSCDKTHTCPPCPAPELLGGPSVFLFPPKPKDT |
| | | | LMISRTPEVTCVVVDVSHEDPEVKFNWYVDGVEVHNAKTKPR |
| | | | EEQYNSTYRVVSVLTVLHQDWLNGKEYKCKVSNKALPAPIEK |
| | | | TISKAKGQPREPQVYTLPPSRDELTKNQVSLWCLVKGFYPSD |
| | | | IAVEWESNGQPENNYKTTPPVLDSDGSFFLYSKLTVDKSRWQ |
| | | | QGNVFSCSVMHEALHNHYTQKSLSLSPGKGGGGSGGGGSGGG |
| | | | GSIWELKKDVYVVELDWYPDAPGEMVVLTCDTPEEDGITWTL |
| | | | DQSSEVLGSGKTLTIQVKAAGDAGQYTCHKGGEVLSHSLLLL |
| | | | HKKEDGIWSTDILKDQKEPKNKTFLRCEAKNYSGRFTCWWLT |
| | | | TISTDLTFSVKSSRGSSDPQGVTCGAATLSAERVRGDNKEYE |
| | | | YSVECQEDSACPAAEESLPIEVMVDAVHKLKYENYTSSFFIR |
| | | | DIIKPDPPKNLQLKPLKNSRQVEVSWEYPDTWSTPHSYFSLT |
| | | | FCVQVQGKSKREKKDRVFTDKTSATVICRKNASISVRAQDRY |
| | | | YSSSWSEWASVPCS |
| | Heavy Chain | 305 | See Table 19. |
| | (Hole) | | |
| | IL-12p35 | | |
| | Light Chain | 245 | See Table 19. |
| BCA327.16 | Heavy Chain | 322 | EVRLVESGGGLVKPGGSLRLSCAASGFTFSNYYMSWIRQAPE |
| See e.g., | (Knob) | | KRLELVSAINSDGGITYYLDTVKGRFTISRDNAKNSLYLQMN |
| FIG. 1 | IL-12p40 | | SLRAEDTALFYCARHRSGYFSMDYWGQGTSVTVSSASTKGPS |
| | (variant K) | | VFPLAPSSKSTSGGTAALGCLVKDYFPEPVTVSWNSGALTSG |
| | | | VHTFPAVLQSSGLYSLSSVVTVPSSSLGTQTYICNVNHKPSN |
| | | | TKVDKKVEPKSCDKTHTCPPCPAPELLGGPSVFLFPPKPKDT |
| | | | LMISRTPEVTCVVVDVSHEDPEVKFNWYVDGVEVHNAKTKPR |
| | | | EEQYNSTYRVVSVLTVLHQDWLNGKEYKCKVSNKALPAPIEK |
| | | | TISKAKGQPREPQVYTLPPSRDELTKNQVSLWCLVKGFYPSD |
| | | | IAVEWESNGQPENNYKTTPPVLDSDGSFFLYSKLTVDKSRWQ |
| | | | QGNVFSCSVMHEALHNHYTQKSLSLSPGKGGGGSGGGGSGGG |
| | | | GSIWELKKDVYVVELDWYPDAPGEMVVLTCDTPEEDGITWTL |
| | | | DQSSEVLGSGKTLTIQVKAAGDAGQYTCHKGGEVLSHSLLLL |
| | | | HAKEDGIWSTDILKDQKEPKNKTFLRCEAKNYSGRFTCWWLT |
| | | | TISTDLTESVKSSRGSSDPQGVTCGAATLSAERVRGDNKEYE |
| | | | YSVECQEDSACPAAEESLPIEVMVDAVHKLKYENYTSSFFIR |
| | | | DIIKPDPPKNLQLKPLKNSRQVEVSWEYPDTWSTPHSYESLT |
| | | | FCVQVQGKSKREKKDRVFTDKTSATVICRKNASISVRAQDRY |
| | | | YSSSWSEWASVPCS |
| | Heavy Chain | 305 | See Table 19. |
| | (Hole) | | |
| | IL-12p35 | | |
| | Light Chain | 245 | See Table 19. |
| BCA328.16 | Heavy Chain | 323 | EVRLVESGGGLVKPGGSLRLSCAASGFTFSNYYMSWIRQAPE |
| See e.g., | (Knob) | | KRLELVSAINSDGGITYYLDTVKGRFTISRDNAKNSLYLQMN |
| FIG. 1 | IL-12p40 | | SLRAEDTALFYCARHRSGYFSMDYWGQGTSVTVSSASTKGPS |
| | (variant L) | | VFPLAPSSKSTSGGTAALGCLVKDYFPEPVTVSWNSGALTSG |
| | | | VHTFPAVLQSSGLYSLSSVVTVPSSSLGTQTYICNVNHKPSN |
| | | | TKVDKKVEPKSCDKTHTCPPCPAPELLGGPSVFLFPPKPKDT |
| | | | LMISRTPEVTCVVVDVSHEDPEVKFNWYVDGVEVHNAKTKPR |
| | | | EEQYNSTYRVVSVLTVLHQDWLNGKEYKCKVSNKALPAPIEK |
| | | | TISKAKGQPREPQVYTLPPSRDELTKNQVSLWCLVKGFYPSD |
| | | | IAVEWESNGQPENNYKTTPPVLDSDGSFFLYSKLTVDKSRWQ |
| | | | QGNVFSCSVMHEALHNHYTQKSLSLSPGKGGGGSGGGGSGGG |
| | | | GSIWELKKDVYVVELDWYPDAPGEMVVLTCDTPEEDGITWTL |
| | | | DQSSEVLGSGKTLTIQVKAAGDAGQYTCHKGGEVLSHSLLLL |
| | | | HAKEDGIWSTDILKDQKEPKNKTFLRCEAKNYSGRFTCWWLT |
| | | | TISTDLTFSVKSSRGSSDPQGVTCGAATLSAERVRGDNKEYE |
| | | | YSVECQEDSACPAAEESLPIEVMVDAVHALKYENYTSSFFIR |
| | | | DIIKPDPPKNLQLKPLKNSRQVEVSWEYPDTWSTPHSYESLT |
| | | | FCVQVQGKSKREKKDRVFTDKTSATVICRKNASISVRAQDRY |
| | | | YSSSWSEWASVPCS |
| | Heavy Chain | 305 | See Table 19. |
| | (Hole) | | |
| | IL-12p35 | | |
| | Light Chain | 245 | See Table 19. |
| BCA329.16 | Heavy Chain | 324 | EVRLVESGGGLVKPGGSLRLSCAASGFTFSNYYMSWIRQAPE |
| See e.g., | (Knob) | | KRLELVSAINSDGGITYYLDTVKGRFTISRDNAKNSLYLQMN |
| FIG. 1 | IL-12p40 | | SLRAEDTALFYCARHRSGYFSMDYWGQGTSVTVSSASTKGPS |
| | (variant M) | | VFPLAPSSKSTSGGTAALGCLVKDYFPEPVTVSWNSGALTSG |
| | | | VHTFPAVLQSSGLYSLSSVVTVPSSSLGTQTYICNVNHKPSN |
| | | | TKVDKKVEPKSCDKTHTCPPCPAPELLGGPSVFLFPPKPKDT |
| | | | LMISRTPEVTCVVVDVSHEDPEVKFNWYVDGVEVHNAKTKPR |
| | | | EEQYNSTYRVVSVLTVLHQDWLNGKEYKCKVSNKALPAPIEK |
| | | | TISKAKGQPREPQVYTLPPSRDELTKNQVSLWCLVKGFYPSD |
| | | | IAVEWESNGQPENNYKTTPPVLDSDGSFFLYSKLTVDKSRWQ |
| | | | QGNVFSCSVMHEALHNHYTQKSLSLSPGKGGGGSGGGGSGGG |
| | | | GSIWELKKDVYVVELDWYAAAPGEMVVLTCDTPEEDGITWTL |
| | | | DQSSEVLGSGKTLTIQVKAAGDAGQYTCHKGGEVLSHSLLLL |
| | | | HKKEDGIWSTDILKDQKEPKNKTFLRCEAKNYSGRFTCWWLT |
| | | | TISTDLTFSVKSSRGSSDPQGVTCGAATLSAERVRGDNKEYE |
| | | | YSVECQEDSACPAAEESLPIEVMVDAVHKLKYENYTSSFFIR |
| | | | DIIKPDPPKNLQLKPLKNSRQVEVSWEYPDTWSTPHSYFSLT |
| | | | FCVQVQGKSKREKKDRVFTDKTSATVICRKNASISVRAQDRY |
| | | | YSSSWSEWASVPCS |
| | Heavy Chain | 305 | See Table 19. |
| | (Hole) | | |
| | IL-12p35 | | |
| | Light Chain | 245 | See Table 19. |
| BCA330.16 | Heavy Chain | 325 | EVRLVESGGGLVKPGGSLRLSCAASGFTFSNYYMSWIRQAPE |
| (BCA376) | (Knob) | | KRLELVSAINSDGGITYYLDTVKGRFTISRDNAKNSLYLQMN |
| See e.g., | IL-12p40 | | SLRAEDTALFYCARHRSGYFSMDYWGQGTSVTVSSASTKGPS |
| FIG. 1 | (variant N) | | VFPLAPSSKSTSGGTAALGCLVKDYFPEPVTVSWNSGALTSG |
| | | | VHTFPAVLQSSGLYSLSSVVTVPSSSLGTQTYICNVNHKPSN |
| | | | TKVDKKVEPKSCDKTHTCPPCPAPELLGGPSVFLFPPKPKDT |
| | | | LMISRTPEVTCVVVDVSHEDPEVKFNWYVDGVEVHNAKTKPR |
| | | | EEQYNSTYRVVSVLTVLHQDWLNGKEYKCKVSNKALPAPIEK |
| | | | TISKAKGQPREPQVYTLPPSRDELTKNQVSLWCLVKGFYPSD |
| | | | IAVEWESNGQPENNYKTTPPVLDSDGSFFLYSKLTVDKSRWQ |
| | | | QGNVFSCSVMHEALHNHYTQKSLSLSPGKGGGGSGGGGSGGG |
| | | | GSIWELKKDVYVVELDAYPDAPGEMVVLTCDTPEEDGITWTL |
| | | | DQSSEVLGSGKTLTIQVKEAGDAGQYTCHKGGEVLSHSLLLL |
| | | | HKKEDGIWSTDILKDQKEPKNKTFLRCEAKNYSGRFTCWWLT |
| | | | TISTDLTFSVKSSRGSSDPQGVTCGAATLSAERVRGDNKEYE |
| | | | YSVECQEDSACPAAEESLPIEVMVDAVHALKYENYTSSFFIR |
| | | | DIIKPDPPKNLQLKPLKNSRQVEVSWEYPDTWSTPHSYESLT |
| | | | FCVQVQGKSKREKKDRVFTDKTSATVICRKNASISVRAQDRY |
| | | | YSSSWSEWASVPCS |
| | Heavy Chain | 305 | See Table 19. |
| | (Hole) | | |
| | IL-12p35 | | |
| | Light Chain | 245 | See Table 19. |
| BCA331.16 | Heavy Chain | 326 | EVRLVESGGGLVKPGGSLRLSCAASGFTFSNYYMSWIRQAPE |
| (BCA356) | (Knob) | | KRLELVSAINSDGGITYYLDTVKGRFTISRDNAKNSLYLQMN |
| See e.g., | IL-12p40 | | SLRAEDTALFYCARHRSGYFSMDYWGQGTSVTVSSASTKGPS |
| FIG. 1 | (variant O) | | VFPLAPSSKSTSGGTAALGCLVKDYFPEPVTVSWNSGALTSG |
| | | | VHTFPAVLQSSGLYSLSSVVTVPSSSLGTQTYICNVNHKPSN |
| | | | TKVDKKVEPKSCDKTHTCPPCPAPELLGGPSVFLFPPKPKDT |
| | | | LMISRTPEVTCVVVDVSHEDPEVKFNWYVDGVEVHNAKTKPR |
| | | | EEQYNSTYRVVSVLTVLHQDWLNGKEYKCKVSNKALPAPIEK |
| | | | TISKAKGQPREPQVYTLPPSRDELTKNQVSLWCLVKGFYPSD |
| | | | IAVEWESNGQPENNYKTTPPVLDSDGSFFLYSKLTVDKSRWQ |
| | | | QGNVFSCSVMHEALHNHYTQKSLSLSPGKGGGGSGGGGSGGG |
| | | | GSIWELKKDVYVVELDAYPDAPGEMVVLTCDTPEEDGITWTL |
| | | | DQSSEVLGSGKTLTIQVKEAGDAGQYTCHKGGEVLSHSLLLL |
| | | | HKKEDGIWSTDILKDQKEPKNKTFLRCEAKNYSGRFTCWWLT |
| | | | TISTDLTFSVKSSRGSSDPQGVTCGAATLSAERVRGDNKEYE |
| | | | YSVECQEDSACPAAEESLPIEVMVDAVHKLAYENYTSSFFIR |
| | | | DIIKPDPPKNLQLKPLKNSRQVEVSWEYPDTWSTPHSYESLT |
| | | | FCVQVQGKSKREKKDRVFTDKTSATVICRKNASISVRAQDRY |
| | | | YSSSWSEWASVPCS |
| | Heavy Chain | 305 | See Table 19. |
| | (Hole) | | |
| | IL-12p35 | | |
| | Light Chain | 245 | See Table 19. |
| BCA332.16 | Heavy Chain | 327 | EVRLVESGGGLVKPGGSLRLSCAASGFTFSNYYMSWIRQAPE |
| See e.g., | (Knob) | | KRLELVSAINSDGGITYYLDTVKGRFTISRDNAKNSLYLQMN |
| FIG. 1 | IL-12p40 | | SLRAEDTALFYCARHRSGYFSMDYWGQGTSVTVSSASTKGPS |
| | (variant P) | | VFPLAPSSKSTSGGTAALGCLVKDYFPEPVTVSWNSGALTSG |
| | | | VHTFPAVLQSSGLYSLSSVVTVPSSSLGTQTYICNVNHKPSN |
| | | | TKVDKKVEPKSCDKTHTCPPCPAPELLGGPSVFLFPPKPKDT |
| | | | LMISRTPEVTCVVVDVSHEDPEVKFNWYVDGVEVHNAKTKPR |
| | | | EEQYNSTYRVVSVLTVLHQDWLNGKEYKCKVSNKALPAPIEK |
| | | | TISKAKGQPREPQVYTLPPSRDELTKNQVSLWCLVKGFYPSD |
| | | | IAVEWESNGQPENNYKTTPPVLDSDGSFFLYSKLTVDKSRWQ |
| | | | QGNVFSCSVMHEALHNHYTQKSLSLSPGKGGGGSGGGGSGGG |
| | | | GSIWELKKDVYVVELDWYPDAPGEMVVLTCDTPEEDGITWTL |
| | | | DQSSEVLGSGKTLTIQVKEFGDAGQYTCHKGGEVLSHSLLLL |
| | | | HAKEDGIWSTDILKDQKEPKNKTFLRCEAKNYSGRFTCWWLT |
| | | | TISTDLTFSVKSSRGSSDPQGVTCGAATLSAERVRGDNKEYE |
| | | | YSVECQEDSACPAAEESLPIEVMVDAVHALAYENYTSSFFIR |
| | | | DIIKPDPPKNLQLKPLKNSRQVEVSWEYPDTWSTPHSYFSLT |
| | | | FCVQVQGKSKREKKDRVFTDKTSATVICRKNASISVRAQDRY |
| | | | YSSSWSEWASVPCS |
| | Heavy Chain | 305 | See Table 19. |
| | (Hole) | | |
| | IL-12p35 | | |
| | Light Chain | 245 | See Table 19. |
| BCA333.16 | Heavy Chain | 328 | EVRLVESGGGLVKPGGSLRLSCAASGFTFSNYYMSWIRQAPE |
| See e.g., | (Knob) | | KRLELVSAINSDGGITYYLDTVKGRFTISRDNAKNSLYLQMN |
| FIG. 1 | IL-12p40 | | SLRAEDTALFYCARHRSGYFSMDYWGQGTSVTVSSASTKGPS |
| | (variant Q) | | VFPLAPSSKSTSGGTAALGCLVKDYFPEPVTVSWNSGALTSG |
| | | | VHTFPAVLQSSGLYSLSSVVTVPSSSLGTQTYICNVNHKPSN |
| | | | TKVDKKVEPKSCDKTHTCPPCPAPELLGGPSVFLFPPKPKDT |
| | | | LMISRTPEVTCVVVDVSHEDPEVKFNWYVDGVEVHNAKTKPR |
| | | | EEQYNSTYRVVSVLTVLHQDWLNGKEYKCKVSNKALPAPIEK |
| | | | TISKAKGQPREPQVYTLPPSRDELTKNQVSLWCLVKGFYPSD |
| | | | IAVEWESNGQPENNYKTTPPVLDSDGSFFLYSKLTVDKSRWQ |
| | | | QGNVFSCSVMHEALHNHYTQKSLSLSPGKGGGGSGGGGSGGG |
| | | | GSIWELKKDVYVVELDAYPDAPGEMVVLTCDTPEEDGITWTL |
| | | | DQSSEVLGSGKTLTIQVKEAGDAGQYTCHKGGEVLSHSLLLL |
| | | | HAKEDGIWSTDILKDQKEPKNKTFLRCEAKNYSGRFTCWWLT |
| | | | TISTDLTFSVKSSRGSSDPQGVTCGAATLSAERVRGDNKEYE |
| | | | YSVECQEDSACPAAEESLPIEVMVDAVHKLAYENYTSSFFIR |
| | | | DIIKPDPPKNLQLKPLKNSRQVEVSWEYPDTWSTPHSYFSLT |
| | | | FCVQVQGKSKREKKDRVFTDKTSATVICRKNASISVRAQDRY |
| | | | YSSSWSEWASVPCS |
| | Heavy Chain | 305 | See Table 19. |
| | (Hole) | | |
| | IL-12p35 | | |
| | Light Chain | 245 | See Table 19. |
| BCA334.16 | Heavy Chain | 329 | EVRLVESGGGLVKPGGSLRLSCAASGFTFSNYYMSWIRQAPE |
| See e.g., | (Knob) | | KRLELVSAINSDGGITYYLDTVKGRFTISRDNAKNSLYLQMN |
| FIG. 1 | IL-12p40 | | SLRAEDTALFYCARHRSGYFSMDYWGQGTSVTVSSASTKGPS |
| | (variant R) | | VFPLAPSSKSTSGGTAALGCLVKDYFPEPVTVSWNSGALTSG |
| | | | VHTFPAVLQSSGLYSLSSVVTVPSSSLGTQTYICNVNHKPSN |
| | | | TKVDKKVEPKSCDKTHTCPPCPAPELLGGPSVFLFPPKPKDT |
| | | | LMISRTPEVTCVVVDVSHEDPEVKFNWYVDGVEVHNAKTKPR |
| | | | EEQYNSTYRVVSVLTVLHQDWLNGKEYKCKVSNKALPAPIEK |
| | | | TISKAKGQPREPQVYTLPPSRDELTKNQVSLWCLVKGFYPSD |
| | | | IAVEWESNGQPENNYKTTPPVLDSDGSFFLYSKLTVDKSRWQ |
| | | | QGNVFSCSVMHEALHNHYTQKSLSLSPGKGGGGSGGGGSGGG |
| | | | GSIWELKKDVYVVELDWYPDAPGEMVVLTCDTPEEDGITWTL |
| | | | DQSSEVLGSGKTLTIQVKEFGDAGQYTCHKGGEVLSHSLLLL |
| | | | HKKEDGIWSTDILKDQKEPKNKTFLRCEAKNYSGRFTCWWLT |
| | | | TISTDLTFSVKSSRGSSDPQGVTCGAATLSAERVRGDNKEYE |
| | | | YSVECQEDSACPAAEESLPIEVMVDAVAALAYENYTSSFFIR |
| | | | DIIKPDPPKNLQLKPLKNSRQVEVSWEYPDTWSTPHSYFSLT |
| | | | FCVQVQGKSKREKKDRVFTDKTSATVICRKNASISVRAQDRY |
| | | | YSSSWSEWASVPCS |
| | Heavy Chain | 305 | See Table 19. |
| | (Hole) | | |
| | IL-12p35 | | |
| | Light Chain | 245 | See Table 19. |
| BCA335.16 | Heavy Chain | 330 | EVRLVESGGGLVKPGGSLRLSCAASGFTFSNYYMSWIRQAPE |
| (BCA373) | (Knob) | | KRLELVSAINSDGGITYYLDTVKGRFTISRDNAKNSLYLQMN |
| See e.g., | hIL-12p40 | | SLRAEDTALFYCARHRSGYFSMDYWGQGTSVTVSSASTKGPS |
| FIG. 1 | Variant T | | VFPLAPSSKSTSGGTAALGCLVKDYFPEPVTVSWNSGALTSG |
| | | | VHTFPAVLQSSGLYSLSSVVTVPSSSLGTQTYICNVNHKPSN |
| | | | TKVDKKVEPKSCDKTHTCPPCPAPELLGGPSVFLFPPKPKDT |
| | | | LMISRTPEVTCVVVDVSHEDPEVKFNWYVDGVEVHNAKTKPR |
| | | | EEQYNSTYRVVSVLTVLHQDWLNGKEYKCKVSNKALPAPIEK |
| | | | TISKAKGQPREPQVYTLPPSRDELTKNQVSLWCLVKGFYPSD |
| | | | IAVEWESNGQPENNYKTTPPVLDSDGSFFLYSKLTVDKSRWQ |
| | | | QGNVFSCSVMHEALHNHYTQKSLSLSPGKGGGGSGGGGSGGG |
| | | | GSIWELKKDVYVVELDAYPDAPGEMVVLTCDTPEEDGITWTL |
| | | | DQSSEVLGSGKTLTIQVKEAGDAGQYTCHKGGEVLSHSLLLL |
| | | | HKKEDGIWSTDILKDQKEPKNKTFLRCEAKNYSGRFTCWWLT |
| | | | TISTDLTFSVKSSRGSSDPQGVTCGAATLSAERVRGDNKEYE |
| | | | YSVECQEDSACPAAEESLPIEVMVDAVHKLKYENYTSSFFIR |
| | | | DIIKPDPPKNLQLKPLKNSRQVEVSWEYPDTWSTPHSYFSLT |
| | | | FCVQVQGKSKREKKDRVFTDKTSATVICRKNASISVRAQDRY |
| | | | YSSSWSEWASVPCS |
| | Heavy Chain | 305 | See Table 19. |
| | (Hole) | | |
| | IL-12p35 | | |
| | Light Chain | 245 | See Table 19. |
| BCA336.16 | Heavy Chain | 331 | EVRLVESGGGLVKPGGSLRLSCAASGFTFSNYYMSWIRQAPE |
| (BCA374) | (Knob) | | KRLELVSAINSDGGITYYLDTVKGRFTISRDNAKNSLYLQMN |
| See e.g., | hIL-12p40 | | SLRAEDTALFYCARHRSGYFSMDYWGQGTSVTVSSASTKGPS |
| FIG. 1 | Variant U | | VFPLAPSSKSTSGGTAALGCLVKDYFPEPVTVSWNSGALTSG |
| | | | VHTFPAVLQSSGLYSLSSVVTVPSSSLGTQTYICNVNHKPSN |
| | | | TKVDKKVEPKSCDKTHTCPPCPAPELLGGPSVFLFPPKPKDT |
| | | | LMISRTPEVTCVVVDVSHEDPEVKFNWYVDGVEVHNAKTKPR |
| | | | EEQYNSTYRVVSVLTVLHQDWLNGKEYKCKVSNKALPAPIEK |
| | | | TISKAKGQPREPQVYTLPPSRDELTKNQVSLWCLVKGFYPSD |
| | | | IAVEWESNGQPENNYKTTPPVLDSDGSFFLYSKLTVDKSRWQ |
| | | | QGNVFSCSVMHEALHNHYTQKSLSLSPGKGGGGSGGGGSGGG |
| | | | GSIWELKKDVYVVELDAYPDAPGEMVVLTCDTPEEDGITWTL |
| | | | DQSSEVLGSGKTLTIQVKEFGDAGQYTCHKGGEVLSHSLLLL |
| | | | HKKEDGIWSTDILKDQKEPKNKTFLRCEAKNYSGRFTCWWLT |
| | | | TISTDLTFSVKSSRGSSDPQGVTCGAATLSAERVRGDNKEYE |
| | | | YSVECQEDSACPAAEESLPIEVMVDAVHALKYENYTSSFFIR |
| | | | DIIKPDPPKNLQLKPLKNSRQVEVSWEYPDTWSTPHSYFSLT |
| | | | FCVQVQGKSKREKKDRVFTDKTSATVICRKNASISVRAQDRY |
| | | | YSSSWSEWASVPCS |
| | Heavy Chain | 305 | See Table 19. |
| | (Hole) | | |
| | IL-12p35 | | |
| | Light Chain | 245 | See Table 19. |
| BCA337.16 | Heavy Chain | 332 | EVRLVESGGGLVKPGGSLRLSCAASGFTFSNYYMSWIRQAPE |
| (BCA375) | (Knob) | | KRLELVSAINSDGGITYYLDTVKGRFTISRDNAKNSLYLQMN |
| See e.g., | hIL-12p40 | | SLRAEDTALFYCARHRSGYFSMDYWGQGTSVTVSSASTKGPS |
| FIG. 1 | Variant V | | VFPLAPSSKSTSGGTAALGCLVKDYFPEPVTVSWNSGALTSG |
| | | | VHTFPAVLQSSGLYSLSSVVTVPSSSLGTQTYICNVNHKPSN |
| | | | TKVDKKVEPKSCDKTHTCPPCPAPELLGGPSVFLFPPKPKDT |
| | | | LMISRTPEVTCVVVDVSHEDPEVKFNWYVDGVEVHNAKTKPR |
| | | | EEQYNSTYRVVSVLTVLHQDWLNGKEYKCKVSNKALPAPIEK |
| | | | TISKAKGQPREPQVYTLPPSRDELTKNQVSLWCLVKGFYPSD |
| | | | IAVEWESNGQPENNYKTTPPVLDSDGSFFLYSKLTVDKSRWQ |
| | | | QGNVFSCSVMHEALHNHYTQKSLSLSPGKGGGGSGGGGSGGG |
| | | | GSIWELKKDVYVVELDAYPDAPGEMVVLTCDTPEEDGITWTL |
| | | | DQSSEVLGSGKTLTIQVKEFGDAGQYTCHKGGEVLSHSLLLL |
| | | | HKKEDGIWSTDILKDQKEPKNKTFLRCEAKNYSGRFTCWWLT |
| | | | TISTDLTFSVKSSRGSSDPQGVTCGAATLSAERVRGDNKEYE |
| | | | YSVECQEDSACPAAEESLPIEVMVDAVHKLAYENYTSSFFIR |
| | | | DIIKPDPPKNLQLKPLKNSRQVEVSWEYPDTWSTPHSYFSLT |
| | | | FCVQVQGKSKREKKDRVFTDKTSATVICRKNASISVRAQDRY |
| | | | YSSSWSEWASVPCS |
| | Heavy Chain | 305 | See Table 19. |
| | (Hole) | | |
| | IL-12p35 | | |
| | Light Chain | 245 | See Table 19. |
| BCA338.16 | Heavy Chain | 333 | EVRLVESGGGLVKPGGSLRLSCAASGFTFSNYYMSWIRQAPE |
| See e.g., | (Knob) | | KRLELVSAINSDGGITYYLDTVKGRFTISRDNAKNSLYLQMN |
| FIG. 1 | hIL-12p40 | | SLRAEDTALFYCARHRSGYFSMDYWGQGTSVTVSSASTKGPS |
| | Variant W | | VFPLAPSSKSTSGGTAALGCLVKDYFPEPVTVSWNSGALTSG |
| | | | VHTFPAVLQSSGLYSLSSVVTVPSSSLGTQTYICNVNHKPSN |
| | | | TKVDKKVEPKSCDKTHTCPPCPAPELLGGPSVFLFPPKPKDT |
| | | | LMISRTPEVTCVVVDVSHEDPEVKFNWYVDGVEVHNAKTKPR |
| | | | EEQYNSTYRVVSVLTVLHQDWLNGKEYKCKVSNKALPAPIEK |
| | | | TISKAKGQPREPQVYTLPPSRDELTKNQVSLWCLVKGFYPSD |
| | | | IAVEWESNGQPENNYKTTPPVLDSDGSFFLYSKLTVDKSRWQ |
| | | | QGNVFSCSVMHEALHNHYTQKSLSLSPGKGGGGSGGGGSGGG |
| | | | GSIWELKKDVYVVELDAYPDAPGEMVVLTCDTPEEDGITWTL |
| | | | DQSSEVLGSGKTLTIQVKEFGDAGQYTCHKGGEVLSHSLLLL |
| | | | HAKEDGIWSTDILKDQKEPKNKTFLRCEAKNYSGRFTCWWLT |
| | | | TISTDLTFSVKSSRGSSDPQGVTCGAATLSAERVRGDNKEYE |
| | | | YSVECQEDSACPAAEESLPIEVMVDAVHKLKYENYTSSFFIR |
| | | | DIIKPDPPKNLQLKPLKNSRQVEVSWEYPDTWSTPHSYESLT |
| | | | FCVQVQGKSKREKKDRVFTDKTSATVICRKNASISVRAQDRY |
| | | | YSSSWSEWASVPCS |
| | Heavy Chain | 305 | See Table 19. |
| | (Hole) | | |
| | IL-12p35 | | |
| | Light Chain | 245 | See Table 19. |
| BCA339.16 | Heavy Chain | 334 | EVRLVESGGGLVKPGGSLRLSCAASGFTFSNYYMSWIRQAPE |
| See e.g., | (Knob) | | KRLELVSAINSDGGITYYLDTVKGRFTISRDNAKNSLYLQMN |
| FIG. 1 | hIL-12p40 | | SLRAEDTALFYCARHRSGYFSMDYWGQGTSVTVSSASTKGPS |
| | Variant X | | VFPLAPSSKSTSGGTAALGCLVKDYFPEPVTVSWNSGALTSG |
| | | | VHTFPAVLQSSGLYSLSSVVTVPSSSLGTQTYICNVNHKPSN |
| | | | TKVDKKVEPKSCDKTHTCPPCPAPELLGGPSVFLFPPKPKDT |
| | | | LMISRTPEVTCVVVDVSHEDPEVKFNWYVDGVEVHNAKTKPR |
| | | | EEQYNSTYRVVSVLTVLHQDWLNGKEYKCKVSNKALPAPIEK |
| | | | TISKAKGQPREPQVYTLPPSRDELTKNQVSLWCLVKGFYPSD |
| | | | IAVEWESNGQPENNYKTTPPVLDSDGSFFLYSKLTVDKSRWQ |
| | | | QGNVFSCSVMHEALHNHYTQKSLSLSPGKGGGGSGGGGSGGG |
| | | | GSIWELKKDVYVVELDWYPDAPGEMVVLTCDTPEEDGITWTL |
| | | | DQSSEVLGSGKTLTIQVKEAGDAGQYTCHKGGEVLSHSLLLL |
| | | | HAKEDGIWSTDILKDQKEPKNKTFLRCEAKNYSGRFTCWWLT |
| | | | TISTDLTFSVKSSRGSSDPQGVTCGAATLSAERVRGDNKEYE |
| | | | YSVECQEDSACPAAEESLPIEVMVDAVHKLKYENYTSSFFIR |
| | | | DIIKPDPPKNLQLKPLKNSRQVEVSWEYPDTWSTPHSYFSLT |
| | | | FCVQVQGKSKREKKDRVFTDKTSATVICRKNASISVRAQDRY |
| | | | YSSSWSEWASVPCS |
| | Heavy Chain | 305 | See Table 19. |
| | (Hole) | | |
| | IL-12p35 | | |
| | Light Chain | 245 | See Table 19. |
| BCA340.16 | Heavy Chain | 335 | EVRLVESGGGLVKPGGSLRLSCAASGFTFSNYYMSWIRQAPE |
| See e.g., | (Knob) | | KRLELVSAINSDGGITYYLDTVKGRFTISRDNAKNSLYLQMN |
| FIG. 1 | hIL-12p40 | | SLRAEDTALFYCARHRSGYFSMDYWGQGTSVTVSSASTKGPS |
| | Variant Y | | VFPLAPSSKSTSGGTAALGCLVKDYFPEPVTVSWNSGALTSG |
| | | | VHTFPAVLQSSGLYSLSSVVTVPSSSLGTQTYICNVNHKPSN |
| | | | TKVDKKVEPKSCDKTHTCPPCPAPELLGGPSVFLFPPKPKDT |
| | | | LMISRTPEVTCVVVDVSHEDPEVKFNWYVDGVEVHNAKTKPR |
| | | | EEQYNSTYRVVSVLTVLHQDWLNGKEYKCKVSNKALPAPIEK |
| | | | TISKAKGQPREPQVYTLPPSRDELTKNQVSLWCLVKGFYPSD |
| | | | IAVEWESNGQPENNYKTTPPVLDSDGSFFLYSKLTVDKSRWQ |
| | | | QGNVFSCSVMHEALHNHYTQKSLSLSPGKGGGGSGGGGSGGG |
| | | | GSIWELKKDVYVVELDWYPDAPGEMVVLTCDTPEEDGITWTL |
| | | | DQSSEVLGSGKTLTIQVKEAGDAGQYTCHKGGEVLSHSLLLL |
| | | | HKKEDGIWSTDILKDQKEPKNKTFLRCEAKNYSGRFTCWWLT |
| | | | TISTDLTFSVKSSRGSSDPQGVTCGAATLSAERVRGDNKEYE |
| | | | YSVECQEDSACPAAEESLPIEVMVDAVHALKYENYTSSFFIR |
| | | | DIIKPDPPKNLQLKPLKNSRQVEVSWEYPDTWSTPHSYFSLT |
| | | | FCVQVQGKSKREKKDRVFTDKTSATVICRKNASISVRAQDRY |
| | | | YSSSWSEWASVPCS |
| | Heavy Chain | 305 | See Table 19. |
| | (Hole) | | |
| | IL-12p35 | | |
| | Light Chain | 245 | See Table 19. |
| BCA341.16 | Heavy Chain | 336 | EVRLVESGGGLVKPGGSLRLSCAASGFTFSNYYMSWIRQAPE |
| (BCA377) | (Knob) | | KRLELVSAINSDGGITYYLDTVKGRFTISRDNAKNSLYLQMN |
| See e.g., | hIL-12p40 | | SLRAEDTALFYCARHRSGYFSMDYWGQGTSVTVSSASTKGPS |
| FIG. 1 | Variant Z | | VFPLAPSSKSTSGGTAALGCLVKDYFPEPVTVSWNSGALTSG |
| | | | VHTFPAVLQSSGLYSLSSVVTVPSSSLGTQTYICNVNHKPSN |
| | | | TKVDKKVEPKSCDKTHTCPPCPAPELLGGPSVFLFPPKPKDT |
| | | | LMISRTPEVTCVVVDVSHEDPEVKFNWYVDGVEVHNAKTKPR |
| | | | EEQYNSTYRVVSVLTVLHQDWLNGKEYKCKVSNKALPAPIEK |
| | | | TISKAKGQPREPQVYTLPPSRDELTKNQVSLWCLVKGFYPSD |
| | | | IAVEWESNGQPENNYKTTPPVLDSDGSFFLYSKLTVDKSRWQ |
| | | | QGNVFSCSVMHEALHNHYTQKSLSLSPGKGGGGSGGGGSGGG |
| | | | GSIWELKKDVYVVELDWYPDAPGEMVVLTCDTPEEDGITWTL |
| | | | DQSSEVLGSGKTLTIQVKEAGDAGQYTCHKGGEVLSHSLLLL |
| | | | HKKEDGIWSTDILKDQKEPKNKTFLRCEAKNYSGRFTCWWLT |
| | | | TISTDLTFSVKSSRGSSDPQGVTCGAATLSAERVRGDNKEYE |
| | | | YSVECQEDSACPAAEESLPIEVMVDAVHKLAYENYTSSFFIR |
| | | | DIIKPDPPKNLQLKPLKNSRQVEVSWEYPDTWSTPHSYESLT |
| | | | FCVQVQGKSKREKKDRVFTDKTSATVICRKNASISVRAQDRY |
| | | | YSSSWSEWASVPCS |
| | Heavy Chain | 305 | See Table 19. |
| | (Hole) | | |
| | IL-12p35 | | |
| | Light Chain | 245 | See Table 19. |
| BCA342.16 | Heavy Chain | 337 | EVRLVESGGGLVKPGGSLRLSCAASGFTFSNYYMSWIRQAPE |
| (BCA378) | (Knob) | | KRLELVSAINSDGGITYYLDTVKGRFTISRDNAKNSLYLQMN |
| See e.g., | hIL-12p40 | | SLRAEDTALFYCARHRSGYFSMDYWGQGTSVTVSSASTKGPS |
| FIG. 1 | Variant AA | | VFPLAPSSKSTSGGTAALGCLVKDYFPEPVTVSWNSGALTSG |
| | | | VHTFPAVLQSSGLYSLSSVVTVPSSSLGTQTYICNVNHKPSN |
| | | | TKVDKKVEPKSCDKTHTCPPCPAPELLGGPSVFLFPPKPKDT |
| | | | LMISRTPEVTCVVVDVSHEDPEVKFNWYVDGVEVHNAKTKPR |
| | | | EEQYNSTYRVVSVLTVLHQDWLNGKEYKCKVSNKALPAPIEK |
| | | | TISKAKGQPREPQVYTLPPSRDELTKNQVSLWCLVKGFYPSD |
| | | | IAVEWESNGQPENNYKTTPPVLDSDGSFFLYSKLTVDKSRWQ |
| | | | QGNVFSCSVMHEALHNHYTQKSLSLSPGKGGGGSGGGGSGGG |
| | | | GSIWELKKDVYVVELDWYPDAPGEMVVLTCDTPEEDGITWTL |
| | | | DQSSEVLGSGKTLTIQVKEFGDAGQYTCHKGGEVLSHSLLLL |
| | | | HKKEDGIWSTDILKDQKEPKNKTFLRCEAKNYSGRFTCWWLT |
| | | | TISTDLTFSVKSSRGSSDPQGVTCGAATLSAERVRGDNKEYE |
| | | | YSVECQEDSACPAAEESLPIEVMVDAVHALAYENYTSSFFIR |
| | | | DIIKPDPPKNLQLKPLKNSRQVEVSWEYPDTWSTPHSYFSLT |
| | | | FCVQVQGKSKREKKDRVFTDKTSATVICRKNASISVRAQDRY |
| | | | YSSSWSEWASVPCS |
| | Heavy Chain | 305 | See Table 19. |
| | (Hole) | | |
| | IL-12p35 | | |
| | Light Chain | 245 | See Table 19. |
| BCA343.16 | Heavy Chain | 338 | VRLVESGGGLVKPGGSLRLSCAASGFTFSNYYMSWIRQAPEK |
| See e.g., | (Knob) | | RLELVSAINSDGGITYYLDTVKGRFTISRDNAKNSLYLQMNS |
| FIG. 1 | hIL-12p40 | | LRAEDTALFYCARHRSGYFSMDYWGQGTSVTVSSASTKGPSV |
| | Variant BB | | FPLAPSSKSTSGGTAALGCLVKDYFPEPVTVSWNSGALTSGV |
| | | | HTFPAVLQSSGLYSLSSVVTVPSSSLGTQTYICNVNHKPSNT |
| | | | KVDKKVEPKSCDKTHTCPPCPAPELLGGPSVFLFPPKPKDTL |
| | | | MISRTPEVTCVVVDVSHEDPEVKFNWYVDGVEVHNAKTKPRE |
| | | | EQYNSTYRVVSVLTVLHQDWLNGKEYKCKVSNKALPAPIEKT |
| | | | ISKAKGQPREPQVYTLPPSRDELTKNQVSLWCLVKGFYPSDI |
| | | | AVEWESNGQPENNYKTTPPVLDSDGSFFLYSKLTVDKSRWQQ |
| | | | GNVFSCSVMHEALHNHYTQKSLSLSPGKGGGGSGGGGSGGGG |
| | | | SIWELKKDVYVVELDWYPDAPGEMVVLTCDTPEEDGITWTLD |
| | | | QSSEVLGSGKTLTIQVKEFGDAGQYTCHKGGEVLSHSLLLLH |
| | | | AKEDGIWSTDILKDQKEPKNKTFLRCEAKNYSGRFTCWWLTT |
| | | | ISTDLTFSVKSSRGSSDPQGVTCGAATLSAERVRGDNKEYEY |
| | | | SVECQEDSACPAAEESLPIEVMVDAVHALKYENYTSSFFIRD |
| | | | IIKPDPPKNLQLKPLKNSRQVEVSWEYPDTWSTPHSYFSLTF |
| | | | CVQVQGKSKREKKDRVFTDKTSATVICRKNASISVRAQDRYY |
| | | | SSSWSEWASVPCS |
| | Heavy Chain | 305 | See Table 19. |
| | (Hole) | | |
| | IL-12p35 | | |
| | Light Chain | 245 | See Table 19. |
| BCA344.16 | Heavy Chain | 339 | EVRLVESGGGLVKPGGSLRLSCAASGFTFSNYYMSWIRQAPE |
| See e.g., | (Knob) | | KRLELVSAINSDGGITYYLDTVKGRFTISRDNAKNSLYLQMN |
| FIG. 1 | hIL-12p40 | | SLRAEDTALFYCARHRSGYFSMDYWGQGTSVTVSSASTKGPS |
| | Variant CC | | VFPLAPSSKSTSGGTAALGCLVKDYFPEPVTVSWNSGALTSG |
| | | | VHTFPAVLQSSGLYSLSSVVTVPSSSLGTQTYICNVNHKPSN |
| | | | TKVDKKVEPKSCDKTHTCPPCPAPELLGGPSVFLFPPKPKDT |
| | | | LMISRTPEVTCVVVDVSHEDPEVKFNWYVDGVEVHNAKTKPR |
| | | | EEQYNSTYRVVSVLTVLHQDWLNGKEYKCKVSNKALPAPIEK |
| | | | TISKAKGQPREPQVYTLPPSRDELTKNQVSLWCLVKGFYPSD |
| | | | IAVEWESNGQPENNYKTTPPVLDSDGSFFLYSKLTVDKSRWQ |
| | | | QGNVFSCSVMHEALHNHYTQKSLSLSPGKGGGGSGGGGSGGG |
| | | | GSIWELKKDVYVVELDWYPDAPGEMVVLTCDTPEEDGITWTL |
| | | | DQSSEVLGSGKTLTIQVKEFGDAGQYTCHKGGEVLSHSLLLL |
| | | | HAKEDGIWSTDILKDQKEPKNKTFLRCEAKNYSGRFTCWWLT |
| | | | TISTDLTFSVKSSRGSSDPQGVTCGAATLSAERVRGDNKEYE |
| | | | YSVECQEDSACPAAEESLPIEVMVDAVHKLAYENYTSSFFIR |
| | | | DIIKPDPPKNLQLKPLKNSRQVEVSWEYPDTWSTPHSYFSLT |
| | | | FCVQVQGKSKREKKDRVFTDKTSATVICRKNASISVRAQDRY |
| | | | YSSSWSEWASVPCS |
| | Heavy Chain | 305 | See Table 19. |
| | (Hole) | | |
| | IL-12p35 | | |
| | Light Chain | 245 | See Table 19. |
| BCA345.16 | Heavy Chain | 340 | EVRLVESGGGLVKPGGSLRLSCAASGFTFSNYYMSWIRQAPE |
| (BCA369) | (Knob) | | KRLELVSAINSDGGITYYLDTVKGRFTISRDNAKNSLYLQMN |
| See e.g., | hIL-12p40 | | SLRAEDTALFYCARHRSGYFSMDYWGQGTSVTVSSASTKGPS |
| FIG. 1 | Variant DD | | VFPLAPSSKSTSGGTAALGCLVKDYFPEPVTVSWNSGALTSG |
| | | | VHTFPAVLQSSGLYSLSSVVTVPSSSLGTQTYICNVNHKPSN |
| | | | TKVDKKVEPKSCDKTHTCPPCPAPELLGGPSVFLFPPKPKDT |
| | | | LMISRTPEVTCVVVDVSHEDPEVKFNWYVDGVEVHNAKTKPR |
| | | | EEQYNSTYRVVSVLTVLHQDWLNGKEYKCKVSNKALPAPIEK |
| | | | TISKAKGQPREPQVYTLPPSRDELTKNQVSLWCLVKGFYPSD |
| | | | IAVEWESNGQPENNYKTTPPVLDSDGSFFLYSKLTVDKSRWQ |
| | | | QGNVFSCSVMHEALHNHYTQKSLSLSPGKGGGGSGGGGSGGG |
| | | | GSIWELKKDVYVVELDAYPDAPGEMVVLTCDTPEEDGITWTL |
| | | | DQSSEVLGSGKTLTIQVKEFGDAGQYTCHKGGEVLSHSLLLL |
| | | | HKKEDGIWSTDILKDQKEPKNKTFLRCEAKNYSGRFTCWWLT |
| | | | TISTDLTFSVKSSRGSSDPQGVTCGAATLSAERVRGDNKEYE |
| | | | YSVECQEDSACPAAEESLPIEVMVDAVHKLKYENYTSSFFIR |
| | | | DIIKPDPPKNLQLKPLKNSRQVEVSWEYPDTWSTPHSYFSLT |
| | | | FCVQVQGKSKREKKDRVFTDKTSATVICRKNASISVRAQDRY |
| | | | YSSSWSEWASVPCS |
| | Heavy Chain | 305 | See Table 19. |
| | (Hole) | | |
| | IL-12p35 | | |
| | Light Chain | 245 | See Table 19. |
| BCA346.16 | Heavy Chain | 341 | EVRLVESGGGLVKPGGSLRLSCAASGFTFSNYYMSWIRQAPE |
| (BCA370) | (Knob) | | KRLELVSAINSDGGITYYLDTVKGRFTISRDNAKNSLYLQMN |
| See e.g., | hIL-12p40 | | SLRAEDTALFYCARHRSGYFSMDYWGQGTSVTVSSASTKGPS |
| FIG. 1 | Variant EE | | VFPLAPSSKSTSGGTAALGCLVKDYFPEPVTVSWNSGALTSG |
| | | | VHTFPAVLQSSGLYSLSSVVTVPSSSLGTQTYICNVNHKPSN |
| | | | TKVDKKVEPKSCDKTHTCPPCPAPELLGGPSVFLFPPKPKDT |
| | | | LMISRTPEVTCVVVDVSHEDPEVKFNWYVDGVEVHNAKTKPR |
| | | | EEQYNSTYRVVSVLTVLHQDWLNGKEYKCKVSNKALPAPIEK |
| | | | TISKAKGQPREPQVYTLPPSRDELTKNQVSLWCLVKGFYPSD |
| | | | IAVEWESNGQPENNYKTTPPVLDSDGSFFLYSKLTVDKSRWQ |
| | | | QGNVFSCSVMHEALHNHYTQKSLSLSPGKGGGGSGGGGSGGG |
| | | | GSIWELKKDVYVVELDWYPDAPGEMVVLTCDTPEEDGITWTL |
| | | | DQSSEVLGSGKTLTIQVKEAGDAGQYTCHKGGEVLSHSLLLL |
| | | | HKKEDGIWSTDILKDQKEPKNKTFLRCEAKNYSGRFTCWWLT |
| | | | TISTDLTFSVKSSRGSSDPQGVTCGAATLSAERVRGDNKEYE |
| | | | YSVECQEDSACPAAEESLPIEVMVDAVHKLKYENYTSSFFIR |
| | | | DIIKPDPPKNLQLKPLKNSRQVEVSWEYPDTWSTPHSYFSLT |
| | | | FCVQVQGKSKREKKDRVFTDKTSATVICRKNASISVRAQDRY |
| | | | YSSSWSEWASVPCS |
| | Heavy Chain | 305 | See Table 19. |
| | (Hole) | | |
| | IL-12p35 | | |
| | Light Chain | 245 | See Table 19. |
| BCA347.16 | Heavy Chain | 342 | EVRLVESGGGLVKPGGSLRLSCAASGFTFSNYYMSWIRQAPE |
| See e.g., | (Knob) | | KRLELVSAINSDGGITYYLDTVKGRFTISRDNAKNSLYLQMN |
| FIG. 1 | hIL-12p40 | | SLRAEDTALFYCARHRSGYFSMDYWGQGTSVTVSSASTKGPS |
| | Variant FF | | VFPLAPSSKSTSGGTAALGCLVKDYFPEPVTVSWNSGALTSG |
| | | | VHTFPAVLQSSGLYSLSSVVTVPSSSLGTQTYICNVNHKPSN |
| | | | TKVDKKVEPKSCDKTHTCPPCPAPELLGGPSVFLFPPKPKDT |
| | | | LMISRTPEVTCVVVDVSHEDPEVKFNWYVDGVEVHNAKTKPR |
| | | | EEQYNSTYRVVSVLTVLHQDWLNGKEYKCKVSNKALPAPIEK |
| | | | TISKAKGQPREPQVYTLPPSRDELTKNQVSLWCLVKGFYPSD |
| | | | IAVEWESNGQPENNYKTTPPVLDSDGSFFLYSKLTVDKSRWQ |
| | | | QGNVFSCSVMHEALHNHYTQKSLSLSPGKGGGGSGGGGSGGG |
| | | | GSIWELKKDVYVVELDWYPDAPGEMVVLTCDTPEEDGITWTL |
| | | | DQSSEVLGSGKTLTIQVKEFGDAGQYTCHKGGEVLSHSLLLL |
| | | | HAKEDGIWSTDILKDQKEPKNKTELRCEAKNYSGRFTCWWLT |
| | | | TISTDLTFSVKSSRGSSDPQGVTCGAATLSAERVRGDNKEYE |
| | | | YSVECQEDSACPAAEESLPIEVMVDAVHKLKYENYTSSFFIR |
| | | | DIIKPDPPKNLQLKPLKNSRQVEVSWEYPDTWSTPHSYFSLT |
| | | | FCVQVQGKSKREKKDRVFTDKTSATVICRKNASISVRAQDRY |
| | | | YSSSWSEWASVPCS |
| | Heavy Chain | 305 | See Table 19. |
| | (Hole) | | |
| | IL-12p35 | | |
| | Light Chain | 245 | See Table 19. |
| BCA348.16 | Heavy Chain | 343 | EVRLVESGGGLVKPGGSLRLSCAASGFTFSNYYMSWIRQAPE |
| (BCA371) | (Knob) | | KRLELVSAINSDGGITYYLDTVKGRFTISRDNAKNSLYLQMN |
| See e.g., | hIL-12p40 | | SLRAEDTALFYCARHRSGYFSMDYWGQGTSVTVSSASTKGPS |
| FIG. 1 | Variant GG | | VFPLAPSSKSTSGGTAALGCLVKDYFPEPVTVSWNSGALTSG |
| | | | VHTFPAVLQSSGLYSLSSVVTVPSSSLGTQTYICNVNHKPSN |
| | | | TKVDKKVEPKSCDKTHTCPPCPAPELLGGPSVFLFPPKPKDT |
| | | | LMISRTPEVTCVVVDVSHEDPEVKFNWYVDGVEVHNAKTKPR |
| | | | EEQYNSTYRVVSVLTVLHQDWLNGKEYKCKVSNKALPAPIEK |
| | | | TISKAKGQPREPQVYTLPPSRDELTKNQVSLWCLVKGFYPSD |
| | | | IAVEWESNGQPENNYKTTPPVLDSDGSFFLYSKLTVDKSRWQ |
| | | | QGNVFSCSVMHEALHNHYTQKSLSLSPGKGGGGSGGGGSGGG |
| | | | GSIWELKKDVYVVELDWYPDAPGEMVVLTCDTPEEDGITWTL |
| | | | DQSSEVLGSGKTLTIQVKEFGDAGQYTCHKGGEVLSHSLLLL |
| | | | HKKEDGIWSTDILKDQKEPKNKTFLRCEAKNYSGRFTCWWLT |
| | | | TISTDLTFSVKSSRGSSDPQGVTCGAATLSAERVRGDNKEYE |
| | | | YSVECQEDSACPAAEESLPIEVMVDAVHALKYENYTSSFFIR |
| | | | DIIKPDPPKNLQLKPLKNSRQVEVSWEYPDTWSTPHSYFSLT |
| | | | FCVQVQGKSKREKKDRVFTDKTSATVICRKNASISVRAQDRY |
| | | | YSSSWSEWASVPCS |
| | Heavy Chain | 305 | See Table 19. |
| | (Hole) | | |
| | IL-12p35 | | |
| | Light Chain | 245 | See Table 19. |
| BCA349.16 | Heavy Chain | 344 | EVRLVESGGGLVKPGGSLRLSCAASGFTFSNYYMSWIRQAPE |
| (BCA372) | (Knob) | | KRLELVSAINSDGGITYYLDTVKGRFTISRDNAKNSLYLQMN |
| See e.g., | hIL-12p40 | | SLRAEDTALFYCARHRSGYFSMDYWGQGTSVTVSSASTKGPS |
| FIG. 1 | Variant HH | | VFPLAPSSKSTSGGTAALGCLVKDYFPEPVTVSWNSGALTSG |
| | | | VHTFPAVLQSSGLYSLSSVVTVPSSSLGTQTYICNVNHKPSN |
| | | | TKVDKKVEPKSCDKTHTCPPCPAPELLGGPSVFLFPPKPKDT |
| | | | LMISRTPEVTCVVVDVSHEDPEVKFNWYVDGVEVHNAKTKPR |
| | | | EEQYNSTYRVVSVLTVLHQDWLNGKEYKCKVSNKALPAPIEK |
| | | | TISKAKGQPREPQVYTLPPSRDELTKNQVSLWCLVKGFYPSD |
| | | | IAVEWESNGQPENNYKTTPPVLDSDGSFFLYSKLTVDKSRWQ |
| | | | QGNVFSCSVMHEALHNHYTQKSLSLSPGKGGGGSGGGGSGGG |
| | | | GSIWELKKDVYVVELDWYPDAPGEMVVLTCDTPEEDGITWTL |
| | | | DQSSEVLGSGKTLTIQVKEFGDAGQYTCHKGGEVLSHSLLLL |
| | | | HKKEDGIWSTDILKDQKEPKNKTFLRCEAKNYSGRFTCWWLT |
| | | | TISTDLTFSVKSSRGSSDPQGVTCGAATLSAERVRGDNKEYE |
| | | | YSVECQEDSACPAAEESLPIEVMVDAVHKLAYENYTSSFFIR |
| | | | DIIKPDPPKNLQLKPLKNSRQVEVSWEYPDTWSTPHSYFSLT |
| | | | FCVQVQGKSKREKKDRVFTDKTSATVICRKNASISVRAQDRY |
| | | | YSSSWSEWASVPCS |
| | Heavy Chain | 305 | See Table 19. |
| | (Hole) | | |
| | IL-12p35 | | |
| | Light Chain | 245 | See Table 19. |
| BCA315 | Heavy Chain | 345 | EVRLVESGGGLVKPGGSLRLSCAASGFTFSNYYMSWIRQAPE |
| See e.g., | (Knob) | | KRLELVSAINSDGGITYYLDTVKGRFTISRDNAKNSLYLQMN |
| FIG. 2 | scIL-12 | | SLRAEDTALFYCARHRSGYFSMDYWGQGTSVTVSSASTKGPS |
| | | | VFPLAPSSKSTSGGTAALGCLVKDYFPEPVTVSWNSGALTSG |
| | | | VHTFPAVLQSSGLYSLSSVVTVPSSSLGTQTYICNVNHKPSN |
| | | | TKVDKKVEPKSCDKTHTCPPCPAPELLGGPSVFLFPPKPKDT |
| | | | LMISRTPEVTCVVVDVSHEDPEVKFNWYVDGVEVHNAKTKPR |
| | | | EEQYNSTYRVVSVLTVLHQDWLNGKEYKCKVSNKALPAPIEK |
| | | | TISKAKGQPREPQVYTLPPSRDELTKNQVSLWCLVKGFYPSD |
| | | | IAVEWESNGQPENNYKTTPPVLDSDGSFFLYSKLTVDKSRWQ |
| | | | QGNVFSCSVMHEALHNHYTQKSLSLSPGKGGGGSGGGGSGGG |
| | | | GSRNLPVATPDPGMFPCLHHSQNLLRAVSNMLQKARQTLEFY |
| | | | PCTSEEIDHEDITKDKTSTVEACLPLELTKNESCLNSRETSE |
| | | | ITNGSCLASRKTSFMMALCLSSIYEDLKMYQVEFKTMNAKLL |
| | | | MDPKRQIFLDQNMLAVIDELMQALNENSETVPQKSSLEEPDE |
| | | | YKTKIKLCILLHAFRIRAVTIDRVMSYLNASGGGGGGSIWEL |
| | | | KKDVYVVELDWYPDAPGEMVVLTCDTPEEDGITWTLDQSSEV |
| | | | LGSGKTLTIQVKEFGDAGQYTCHKGGEVLSHSLLLLHKKEDG |
| | | | IWSTDILKDQKEPKNKTFLRCEAKNYSGRFTCWWLTTISTDL |
| | | | TFSVKSSRGSSDPQGVTCGAATLSAERVRGDNKEYEYSVECQ |
| | | | EDSACPAAEESLPIEVMVDAVHKLKYENYTSSFFIRDIIKPD |
| | | | PPKNLQLKPLKNSRQVEVSWEYPDTWSTPHSYFSLTFCVQVQ |
| | | | GKSKREKKDRVFTDKTSATVICRKNASISVRAQDRYYSSSWS |
| | | | EWASVPCS |
| | Heavy Chain | 306 | See Table 19. |
| | (Hole) | | |
| | Light Chain | 245 | See Table 19. |
| BCA307 | Heavy Chain | 346 | EVRLVESGGGLVKPGGSLRLSCAASGFTFSNYYMSWIRQAPE |
| See e.g., | (Knob) | | KRLELVSAINSDGGITYYLDTVKGRFTISRDNAKNSLYLQMN |
| FIG. 2 | scIL-12 (IL- | | SLRAEDTALFYCARHRSGYFSMDYWGQGTSVTVSSASTKGPS |
| | 12p35 | | VFPLAPSSKSTSGGTAALGCLVKDYFPEPVTVSWNSGALTSG |
| | Variant A) | | VHTFPAVLQSSGLYSLSSVVTVPSSSLGTQTYICNVNHKPSN |
| | | | TKVDKKVEPKSCDKTHTCPPCPAPELLGGPSVFLFPPKPKDT |
| | | | LMISRTPEVTCVVVDVSHEDPEVKFNWYVDGVEVHNAKTKPR |
| | | | EEQYNSTYRVVSVLTVLHQDWLNGKEYKCKVSNKALPAPIEK |
| | | | TISKAKGQPREPQVYTLPPSRDELTKNQVSLWCLVKGFYPSD |
| | | | IAVEWESNGQPENNYKTTPPVLDSDGSFFLYSKLTVDKSRWQ |
| | | | QGNVFSCSVMHEALHNHYTQKSLSLSPGKGGGGSGGGGSGGG |
| | | | GSRNLPVATPDPGMFPCLHHSQNLLRAVSNMLQKNESCLNSR |
| | | | ETSFITNGSCLASRKTSEMMALCLSSIYEDLKMYQVEFKTMN |
| | | | AKLLMDPKRQIFLDQNMLAVIDELMQALNENSETVPQKSSLE |
| | | | EPDFYKTKIKLCILLHAFRIRAVTIDRVMSYLNASGGGGGGS |
| | | | IWELKKDVYVVELDWYPDAPGEMVVLTCDTPEEDGITWTLDQ |
| | | | SSEVLGSGKTLTIQVKEFGDAGQYTCHKGGEVLSHSLLLLHK |
| | | | KEDGIWSTDILKDQKEPKNKTFLRCEAKNYSGRFTCWWLTTI |
| | | | STDLTFSVKSSRGSSDPQGVTCGAATLSAERVRGDNKEYEYS |
| | | | VECQEDSACPAAEESLPIEVMVDAVHKLKYENYTSSFFIRDI |
| | | | IKPDPPKNLQLKPLKNSRQVEVSWEYPDTWSTPHSYESLTFC |
| | | | VQVQGKSKREKKDRVFTDKTSATVICRKNASISVRAQDRYYS |
| | | | SSWSEWASVPCS |
| | Heavy Chain | 306 | See Table 19. |
| | (Hole) | | |
| | Light Chain | 245 | See Table 19. |
| BCA308 | Heavy Chain | 347 | EVRLVESGGGLVKPGGSLRLSCAASGFTFSNYYMSWIRQAPE |
| See e.g., | (Knob) | | KRLELVSAINSDGGITYYLDTVKGRFTISRDNAKNSLYLQMN |
| FIG. 2 | scIL-12 (IL- | | SLRAEDTALFYCARHRSGYFSMDYWGQGTSVTVSSASTKGPS |
| | 12p35 | | VFPLAPSSKSTSGGTAALGCLVKDYFPEPVTVSWNSGALTSG |
| | Variant B) | | VHTFPAVLQSSGLYSLSSVVTVPSSSLGTQTYICNVNHKPSN |
| | | | TKVDKKVEPKSCDKTHTCPPCPAPELLGGPSVFLFPPKPKDT |
| | | | LMISRTPEVTCVVVDVSHEDPEVKFNWYVDGVEVHNAKTKPR |
| | | | EEQYNSTYRVVSVLTVLHQDWLNGKEYKCKVSNKALPAPIEK |
| | | | TISKAKGQPREPQVYTLPPSRDELTKNQVSLWCLVKGFYPSD |
| | | | IAVEWESNGQPENNYKTTPPVLDSDGSFFLYSKLTVDKSRWQ |
| | | | QGNVFSCSVMHEALHNHYTQKSLSLSPGKGGGGSGGGGSGGG |
| | | | GSRNLPVATPDPGMFPCLHHSQNLLRAVSNMLQKARQTLKHY |
| | | | SCTSEEIDHEDITKDKTSTVEACLPLELTKNESCLNSRETSE |
| | | | ITNGSCLASRKTSFMMALCLSSIYEDLKMYQVEFKTMNAKLL |
| | | | MDPHRQIFLDQNMLAVIDELMQALNENSETVPQKSSLEEPDP |
| | | | YKTKIKLCILLHAFRIRAVTIDRVMSYLNASGGGGGGSIWEL |
| | | | KKDVYVVELDWYPDAPGEMVVLTCDTPEEDGITWTLDQSSEV |
| | | | LGSGKTLTIQVKEFGDAGQYTCHKGGEVLSHSLLLLHKKEDG |
| | | | IWSTDILKDQKEPKNKTFLRCEAKNYSGRFTCWWLTTISTDL |
| | | | TFSVKSSRGSSDPQGVTCGAATLSAERVRGDNKEYEYSVECQ |
| | | | EDSACPAAEESLPIEVMVDAVHKLKYENYTSSFFIRDIIKPD |
| | | | PPKNLQLKPLKNSRQVEVSWEYPDTWSTPHSYFSLTFCVQVQ |
| | | | GKSKREKKDRVFTDKTSATVICRKNASISVRAQDRYYSSSWS |
| | | | EWASVPCS |
| | Heavy Chain | 306 | See Table 19. |
| | (Hole) | | |
| | Light Chain | 245 | See Table 19. |
| BCA323: | Heavy Chain | 348 | EVRLVESGGGLVKPGGSLRLSCAASGFTFSNYYMSWIRQAPE |
| See e.g., | (Knob) | | KRLELVSAINSDGGITYYLDTVKGRFTISRDNAKNSLYLQMN |
| FIG. 2 | scIL-12 (IL- | | SLRAEDTALFYCARHRSGYFSMDYWGQGTSVTVSSASTKGPS |
| | 12p35 | | VFPLAPSSKSTSGGTAALGCLVKDYFPEPVTVSWNSGALTSG |
| | Variant C) | | VHTFPAVLQSSGLYSLSSVVTVPSSSLGTQTYICNVNHKPSN |
| | | | TKVDKKVEPKSCDKTHTCPPCPAPELLGGPSVFLFPPKPKDT |
| | | | LMISRTPEVTCVVVDVSHEDPEVKFNWYVDGVEVHNAKTKPR |
| | | | EEQYNSTYRVVSVLTVLHQDWLNGKEYKCKVSNKALPAPIEK |
| | | | TISKAKGQPREPQVYTLPPSRDELTKNQVSLWCLVKGFYPSD |
| | | | IAVEWESNGQPENNYKTTPPVLDSDGSFFLYSKLTVDKSRWQ |
| | | | QGNVFSCSVMHEALHNHYTQKSLSLSPGKGGGGSGGGGSGGG |
| | | | GSRNLPVATPDPGMFPCLHHSQNLLRAVSNMLQKARQTLEFY |
| | | | PCTSEEIDHEDITKDKTSTVEACLPLELTKNESCLNSRETSF |
| | | | ITNGSCLASRKTSEMMALCLSSIYEDLKMYQVEFKTMNAKLL |
| | | | MDPKRQIFLDQNMLAVIDELMQALNENSETVPQKSSLEEPDA |
| | | | AKTKIKLCILLHAFRIRAVTIDRVMSYLNASGGGGGGSIWEL |
| | | | KKDVYVVELDWYPDAPGEMVVLTCDTPEEDGITWTLDQSSEV |
| | | | LGSGKTLTIQVKEFGDAGQYTCHKGGEVLSHSLLLLHKKEDG |
| | | | IWSTDILKDQKEPKNKTFLRCEAKNYSGRFTCWWLTTISTDL |
| | | | TFSVKSSRGSSDPQGVTCGAATLSAERVRGDNKEYEYSVECQ |
| | | | EDSACPAAEESLPIEVMVDAVHKLKYENYTSSFFIRDIIKPD |
| | | | PPKNLQLKPLKNSRQVEVSWEYPDTWSTPHSYFSLTFCVQVQ |
| | | | GKSKREKKDRVFTDKTSATVICRKNASISVRAQDRYYSSSWS |
| | | | EWASVPCS |
| | Heavy Chain | 306 | See Table 19. |
| | (Hole) | | |
| | Light Chain | 245 | See Table 19. |
| BCA324 | Heavy Chain | 349 | EVRLVESGGGLVKPGGSLRLSCAASGFTFSNYYMSWIRQAPE |
| See e.g., | (Knob) | | KRLELVSAINSDGGITYYLDTVKGRFTISRDNAKNSLYLQMN |
| FIG. 2 | scIL-12 (IL- | | SLRAEDTALFYCARHRSGYFSMDYWGQGTSVTVSSASTKGPS |
| | 12p35 | | VFPLAPSSKSTSGGTAALGCLVKDYFPEPVTVSWNSGALTSG |
| | Variant D) | | VHTFPAVLQSSGLYSLSSVVTVPSSSLGTQTYICNVNHKPSN |
| | | | TKVDKKVEPKSCDKTHTCPPCPAPELLGGPSVFLFPPKPKDT |
| | | | LMISRTPEVTCVVVDVSHEDPEVKFNWYVDGVEVHNAKTKPR |
| | | | EEQYNSTYRVVSVLTVLHQDWLNGKEYKCKVSNKALPAPIEK |
| | | | TISKAKGQPREPQVYTLPPSRDELTKNQVSLWCLVKGFYPSD |
| | | | IAVEWESNGQPENNYKTTPPVLDSDGSFFLYSKLTVDKSRWQ |
| | | | QGNVFSCSVMHEALHNHYTQKSLSLSPGKGGGGSGGGGSGGG |
| | | | GSRNLPVATPDPGMFPCLHHSQNLLRAVSNMLQKARQTLEFY |
| | | | PCTSEEIDHEDITKDKTSTVEACLPLELTKNESCLNSRETSE |
| | | | ITNGSCLASRKTSFMMALCLSSIYEDLKMYQVEFKTMNAKLL |
| | | | MDPKRQIFLDQNMLAVIDELMQALNENSETVPQKSSLEEPDA |
| | | | YKTKIKLCILLHAFRIRAVTIDRVMSYLNASGGGGGGSIWEL |
| | | | KKDVYVVELDWYPDAPGEMVVLTCDTPEEDGITWTLDQSSEV |
| | | | LGSGKTLTIQVKEFGDAGQYTCHKGGEVLSHSLLLLHKKEDG |
| | | | IWSTDILKDQKEPKNKTFLRCEAKNYSGRFTCWWLTTISTDL |
| | | | TFSVKSSRGSSDPQGVTCGAATLSAERVRGDNKEYEYSVECQ |
| | | | EDSACPAAEESLPIEVMVDAVHKLKYENYTSSFFIRDIIKPD |
| | | | PPKNLQLKPLKNSRQVEVSWEYPDTWSTPHSYFSLTFCVQVQ |
| | | | GKSKREKKDRVFTDKTSATVICRKNASISVRAQDRYYSSSWS |
| | | | EWASVPCS |
| | Heavy Chain | 306 | See Table 19. |
| | (Hole) | | |
| | Light Chain | 245 | See Table 19. |
| BCA325 | Heavy Chain | 350 | EVRLVESGGGLVKPGGSLRLSCAASGFTFSNYYMSWIRQAPE |
| See e.g., | (Knob) | | KRLELVSAINSDGGITYYLDTVKGRFTISRDNAKNSLYLQMN |
| FIG. 2 | scIL-12 (IL- | | SLRAEDTALFYCARHRSGYFSMDYWGQGTSVTVSSASTKGPS |
| | 12p35 | | VFPLAPSSKSTSGGTAALGCLVKDYFPEPVTVSWNSGALTSG |
| | Variant E) | | VHTFPAVLQSSGLYSLSSVVTVPSSSLGTQTYICNVNHKPSN |
| | | | TKVDKKVEPKSCDKTHTCPPCPAPELLGGPSVFLFPPKPKDT |
| | | | LMISRTPEVTCVVVDVSHEDPEVKFNWYVDGVEVHNAKTKPR |
| | | | EEQYNSTYRVVSVLTVLHQDWLNGKEYKCKVSNKALPAPIEK |
| | | | TISKAKGQPREPQVYTLPPSRDELTKNQVSLWCLVKGFYPSD |
| | | | IAVEWESNGQPENNYKTTPPVLDSDGSFFLYSKLTVDKSRWQ |
| | | | QGNVFSCSVMHEALHNHYTQKSLSLSPGKGGGGSGGGGSGGG |
| | | | GSRNLPVATPDPGMFPCLHHSQNLLRAVSNMLQKARQTLEFY |
| | | | PCTSEEIDHEDITKDKTSTVEACLPLELTKNESCLNSRETSE |
| | | | ITNGSCLASRKTSFMMALCLSSIYEDLKMYQVEFKTMNAKLL |
| | | | MDPKRQIFLDQNMLAVIDELMQALNENSETVPQKSSLEEPDE |
| | | | AKTKIKLCILLHAFRIRAVTIDRVMSYLNASGGGGGGSIWEL |
| | | | KKDVYVVELDWYPDAPGEMVVLTCDTPEEDGITWTLDQSSEV |
| | | | LGSGKTLTIQVKEFGDAGQYTCHKGGEVLSHSLLLLHKKEDG |
| | | | IWSTDILKDQKEPKNKTFLRCEAKNYSGRFTCWWLTTISTDL |
| | | | TFSVKSSRGSSDPQGVTCGAATLSAERVRGDNKEYEYSVECQ |
| | | | EDSACPAAEESLPIEVMVDAVHKLKYENYTSSFFIRDIIKPD |
| | | | PPKNLQLKPLKNSRQVEVSWEYPDTWSTPHSYFSLTFCVQVQ |
| | | | GKSKREKKDRVFTDKTSATVICRKNASISVRAQDRYYSSSWS |
| | | | EWASVPCS |
| | Heavy Chain | 306 | See Table 19. |
| | (Hole) | | |
| | Light Chain | 245 | See Table 19. |
| BCA309 | Heavy Chain | 351 | EVRLVESGGGLVKPGGSLRLSCAASGFTFSNYYMSWIRQAPE |
| See e.g., | (Knob) | | KRLELVSAINSDGGITYYLDTVKGRFTISRDNAKNSLYLQMN |
| FIG. 2 | scIL-12 (IL- | | SLRAEDTALFYCARHRSGYFSMDYWGQGTSVTVSSASTKGPS |
| | 12p40 | | VFPLAPSSKSTSGGTAALGCLVKDYFPEPVTVSWNSGALTSG |
| | Variant A) | | VHTFPAVLQSSGLYSLSSVVTVPSSSLGTQTYICNVNHKPSN |
| | | | TKVDKKVEPKSCDKTHTCPPCPAPELLGGPSVFLFPPKPKDT |
| | | | LMISRTPEVTCVVVDVSHEDPEVKFNWYVDGVEVHNAKTKPR |
| | | | EEQYNSTYRVVSVLTVLHQDWLNGKEYKCKVSNKALPAPIEK |
| | | | TISKAKGQPREPQVYTLPPSRDELTKNQVSLWCLVKGFYPSD |
| | | | IAVEWESNGQPENNYKTTPPVLDSDGSFFLYSKLTVDKSRWQ |
| | | | QGNVFSCSVMHEALHNHYTQKSLSLSPGKGGGGSGGGGSGGG |
| | | | GSRNLPVATPDPGMFPCLHHSQNLLRAVSNMLQKARQTLEFY |
| | | | PCTSEEIDHEDITKDKTSTVEACLPLELTKNESCLNSRETSE |
| | | | ITNGSCLASRKTSFMMALCLSSIYEDLKMYQVEFKTMNAKLL |
| | | | MDPKRQIFLDQNMLAVIDELMQALNENSETVPQKSSLEEPDE |
| | | | YKTKIKLCILLHAFRIRAVTIDRVMSYLNASGGGGGGSIWEL |
| | | | KKDVYVVELDWYPDAPGEMVVLTCDTPEEDGITWTLDQSSEV |
| | | | LGSGKTLTIQVKEFGDAGQYTCHKGGEVLSHSLLLLHKKEDG |
| | | | IWSTDILKDQKEPKNKTFLRCEAKNYSGRFTCWWLTTISTDL |
| | | | TFSVKSSRGSSDPQGVTCGAATLSAERVRGDNKEYEYSVECQ |
| | | | EDSACPAAEESLPIEVMVDAVHKLKYENYTSSFFIRDIIKPD |
| | | | PPKNLQLKPLKNSRQVEVSWEYPDTWSTPHSYFSLTFCVQVQ |
| | | | GASAAEAADAVETDKTSATVICRKNASISVRAQDRYYSSSWS |
| | | | EWASVPCS |
| | Heavy Chain | 306 | See Table 19. |
| | (Hole) | | |
| | Light Chain | 245 | See Table 19. |
| BCA310 | Heavy Chain | 352 | EVRLVESGGGLVKPGGSLRLSCAASGFTFSNYYMSWIRQAPE |
| See e.g., | (Knob) | | KRLELVSAINSDGGITYYLDTVKGRFTISRDNAKNSLYLQMN |
| FIG. 2 | scIL-12 (IL- | | SLRAEDTALFYCARHRSGYFSMDYWGQGTSVTVSSASTKGPS |
| | 12p40 | | VFPLAPSSKSTSGGTAALGCLVKDYFPEPVTVSWNSGALTSG |
| | Variant B) | | VHTFPAVLQSSGLYSLSSVVTVPSSSLGTQTYICNVNHKPSN |
| | | | TKVDKKVEPKSCDKTHTCPPCPAPELLGGPSVFLFPPKPKDT |
| | | | LMISRTPEVTCVVVDVSHEDPEVKFNWYVDGVEVHNAKTKPR |
| | | | EEQYNSTYRVVSVLTVLHQDWLNGKEYKCKVSNKALPAPIEK |
| | | | TISKAKGQPREPQVYTLPPSRDELTKNQVSLWCLVKGFYPSD |
| | | | IAVEWESNGQPENNYKTTPPVLDSDGSFFLYSKLTVDKSRWQ |
| | | | QGNVFSCSVMHEALHNHYTQKSLSLSPGKGGGGSGGGGSGGG |
| | | | GSRNLPVATPDPGMFPCLHHSQNLLRAVSNMLQKARQTLEFY |
| | | | PCTSEEIDHEDITKDKTSTVEACLPLELTKNESCLNSRETSE |
| | | | ITNGSCLASRKTSFMMALCLSSIYEDLKMYQVEFKTMNAKLL |
| | | | MDPKRQIFLDQNMLAVIDELMQALNENSETVPQKSSLEEPDE |
| | | | YKTKIKLCILLHAFRIRAVTIDRVMSYLNASGGGGGGSELRC |
| | | | EAKNYSGRFTCWWLTTISTDLTFSVKSSRGSSDPQGVTCGAA |
| | | | TLSAERVRGDNKEYEYSVECQEDSACPAAEESLPIEVMVDAV |
| | | | HKLKYENYTSSFFIRDIIKPDPPKNLQLKPLKNSRQVEVSWE |
| | | | YPDTWSTPHSYFSLTFCVQVQGKSKREKKDRVFTDKTSATVI |
| | | | CRKNASISVRAQDRYYSSSWSEWASVPCS |
| | Heavy Chain | 306 | See Table 19. |
| | (Hole) | | |
| | Light Chain | 245 | See Table 19. |
| BCA311 | Heavy Chain | 353 | EVRLVESGGGLVKPGGSLRLSCAASGFTFSNYYMSWIRQAPE |
| See e.g., | (Knob) | | KRLELVSAINSDGGITYYLDTVKGRFTISRDNAKNSLYLQMN |
| FIG. 2 | scIL-12 (IL- | | SLRAEDTALFYCARHRSGYFSMDYWGQGTSVTVSSASTKGPS |
| | 12p35 | | VFPLAPSSKSTSGGTAALGCLVKDYFPEPVTVSWNSGALTSG |
| | Variant C) | | VHTFPAVLQSSGLYSLSSVVTVPSSSLGTQTYICNVNHKPSN |
| | | | TKVDKKVEPKSCDKTHTCPPCPAPELLGGPSVFLFPPKPKDT |
| | | | LMISRTPEVTCVVVDVSHEDPEVKFNWYVDGVEVHNAKTKPR |
| | | | EEQYNSTYRVVSVLTVLHQDWLNGKEYKCKVSNKALPAPIEK |
| | | | TISKAKGQPREPQVYTLPPSRDELTKNQVSLWCLVKGFYPSD |
| | | | IAVEWESNGQPENNYKTTPPVLDSDGSFFLYSKLTVDKSRWQ |
| | | | QGNVFSCSVMHEALHNHYTQKSLSLSPGKGGGGSGGGGSGGG |
| | | | GSRNLPVATPDPGMFPCLHHSQNLLRAVSNMLQKARQTLEFY |
| | | | PCTSEEIDHEDITKDKTSTVEACLPLELTKNESCLNSRETSF |
| | | | ITNGSCLASRKTSEMMALCLSSIYEDLKMYQVEFKTMNAKLL |
| | | | MDPKRQIFLDQNMLAVIDELMQALNENSETVPQKSSLEEPDE |
| | | | YKTKIKLCILLHAFRIRAVTIDRVMSYLNASGGGGGGSIWEL |
| | | | KKDVYVVELDWYPDAPGEMVVLTCDTPEEDGITWTLDQSSEV |
| | | | LGSGKTLTIQVKEFGDAGQYTCHKGGEVLSHSLLLLHKKEDG |
| | | | IWSTDILKDQKEPKNKTFLRCEAKNYSGRFTCWWLTTISTDL |
| | | | TFSVKSSRGSSDPQGVTCGAATLSAERVRGDNKEYEYSVECQ |
| | | | EDSACPAAEEIES |
| | Heavy Chain | 306 | See Table 19. |
| | (Hole) | | |
| | Light Chain | 245 | See Table 19. |
| BCA312 | Heavy Chain | 354 | EVRLVESGGGLVKPGGSLRLSCAASGFTFSNYYMSWIRQAPE |
| See e.g., | (Knob) | | KRLELVSAINSDGGITYYLDTVKGRFTISRDNAKNSLYLQMN |
| FIG. 2 | scIL-12 (IL- | | SLRAEDTALFYCARHRSGYFSMDYWGQGTSVTVSSASTKGPS |
| | 12p40 | | VFPLAPSSKSTSGGTAALGCLVKDYFPEPVTVSWNSGALTSG |
| | Variant S) | | VHTFPAVLQSSGLYSLSSVVTVPSSSLGTQTYICNVNHKPSN |
| | | | TKVDKKVEPKSCDKTHTCPPCPAPELLGGPSVFLFPPKPKDT |
| | | | LMISRTPEVTCVVVDVSHEDPEVKFNWYVDGVEVHNAKTKPR |
| | | | EEQYNSTYRVVSVLTVLHQDWLNGKEYKCKVSNKALPAPIEK |
| | | | TISKAKGQPREPQVYTLPPSRDELTKNQVSLWCLVKGFYPSD |
| | | | IAVEWESNGQPENNYKTTPPVLDSDGSFFLYSKLTVDKSRWQ |
| | | | QGNVFSCSVMHEALHNHYTQKSLSLSPGKGGGGSGGGGSGGG |
| | | | GSRNLPVATPDPGMFPCLHHSQNLLRAVSNMLQKARQTLEFY |
| | | | PCTSEEIDHEDITKDKTSTVEACLPLELTKNESCLNSRETSF |
| | | | ITNGSCLASRKTSEMMALCLSSIYEDLKMYQVEFKTMNAKLL |
| | | | MDPKRQIFLDQNMLAVIDELMQALNENSETVPQKSSLEEPDE |
| | | | YKTKIKLCILLHAFRIRAVTIDRVMSYLNASGGGGGGSIWEL |
| | | | KKDVYVVELDWYPDAPGEMVVLTCDTPEEDGITWTLDQSSEV |
| | | | LGSGKTLTIQVKEFGDAGQYTCHKGGEVLSHSLLLLHKKEDG |
| | | | IWSTDILKDQKEPKNKTFLRCEAKNYSGRFTCWWLTTISTDL |
| | | | TFSVKSSRGSSDPQGVTCGAATLSAERVRGDNKEYEYSVECQ |
| | | | EDSACPAAEESLSIEVMVDAVHKLKYENYTSSFFIRDIIKPD |
| | | | PPKNLQLKPLKNSRQVEVSWEYPDTWSTPHSYFSLTFCVQVQ |
| | | | GKSKREKKDRVFTDKTSATVICRKNASISVRAQDRYYSSSWS |
| | | | EWASVPCS |
| | Heavy Chain | 306 | See Table 19. |
| | (Hole) | | |
| | Light Chain | 245 | See Table 19. |
| BCA318 | Heavy Chain | 355 | EVRLVESGGGLVKPGGSLRLSCAASGFTFSNYYMSWIRQAPE |
| See e.g., | (Knob) | | KRLELVSAINSDGGITYYLDTVKGRFTISRDNAKNSLYLQMN |
| FIG. 2 | scIL-12 (IL- | | SLRAEDTALFYCARHRSGYFSMDYWGQGTSVTVSSASTKGPS |
| | 12p40 | | VFPLAPSSKSTSGGTAALGCLVKDYFPEPVTVSWNSGALTSG |
| | Variant E) | | VHTFPAVLQSSGLYSLSSVVTVPSSSLGTQTYICNVNHKPSN |
| | | | TKVDKKVEPKSCDKTHTCPPCPAPELLGGPSVFLFPPKPKDT |
| | | | LMISRTPEVTCVVVDVSHEDPEVKFNWYVDGVEVHNAKTKPR |
| | | | EEQYNSTYRVVSVLTVLHQDWLNGKEYKCKVSNKALPAPIEK |
| | | | TISKAKGQPREPQVYTLPPSRDELTKNQVSLWCLVKGFYPSD |
| | | | IAVEWESNGQPENNYKTTPPVLDSDGSFFLYSKLTVDKSRWQ |
| | | | QGNVFSCSVMHEALHNHYTQKSLSLSPGKGGGGSGGGGSGGG |
| | | | GSRNLPVATPDPGMFPCLHHSQNLLRAVSNMLQKARQTLEFY |
| | | | PCTSEEIDHEDITKDKTSTVEACLPLELTKNESCLNSRETSF |
| | | | ITNGSCLASRKTSFMMALCLSSIYEDLKMYQVEFKTMNAKLL |
| | | | MDPKRQIFLDQNMLAVIDELMQALNENSETVPQKSSLEEPDE |
| | | | YKTKIKLCILLHAFRIRAVTIDRVMSYLNASGGGGGGSIWEL |
| | | | KKDVYVVELDWYPDAPGEMVVLTCDTPEEDGITWTLDQSSEV |
| | | | LGSGKTLTIQVKEFGDAGQYTCHKGGEVLSHSLLLLHKKEDG |
| | | | IWSTDILKDQKEPKNKTFLRCEAKNYSGRFTCWWLTTISTDL |
| | | | TFSVKSSRGSSDPQGVTCGAATLSAERVRGDNKEYEYSVECQ |
| | | | EDSACPAAEEIESIKSMVDAVHKLKYENYTSSFFIRDIIKPD |
| | | | PPKNLQLKPLKNSRQVEVSWEYPDTWSTPHSYFSLTFCVQVQ |
| | | | GKSKREKKDRVFTDKTSATVICRKNASISVRAQDRYYSSSWS |
| | | | EWASVPCS |
| | Heavy Chain | 306 | See Table 19. |
| | (Hole) | | |
| | Light Chain | 245 | See Table 19. |
| BCA319 | Heavy Chain | 356 | EVRLVESGGGLVKPGGSLRLSCAASGFTFSNYYMSWIRQAPE |
| See e.g., | (Knob) | | KRLELVSAINSDGGITYYLDTVKGRFTISRDNAKNSLYLQMN |
| FIG. 2 | scIL-12 (IL- | | SLRAEDTALFYCARHRSGYFSMDYWGQGTSVTVSSASTKGPS |
| | 12p40 | | VFPLAPSSKSTSGGTAALGCLVKDYFPEPVTVSWNSGALTSG |
| | Variant F) | | VHTFPAVLQSSGLYSLSSVVTVPSSSLGTQTYICNVNHKPSN |
| | | | TKVDKKVEPKSCDKTHTCPPCPAPELLGGPSVFLFPPKPKDT |
| | | | LMISRTPEVTCVVVDVSHEDPEVKFNWYVDGVEVHNAKTKPR |
| | | | EEQYNSTYRVVSVLTVLHQDWLNGKEYKCKVSNKALPAPIEK |
| | | | TISKAKGQPREPQVYTLPPSRDELTKNQVSLWCLVKGFYPSD |
| | | | IAVEWESNGQPENNYKTTPPVLDSDGSFFLYSKLTVDKSRWQ |
| | | | QGNVFSCSVMHEALHNHYTQKSLSLSPGKGGGGSGGGGSGGG |
| | | | GSRNLPVATPDPGMFPCLHHSQNLLRAVSNMLQKARQTLEFY |
| | | | PCTSEEIDHEDITKDKTSTVEACLPLELTKNESCLNSRETSE |
| | | | ITNGSCLASRKTSFMMALCLSSIYEDLKMYQVEFKTMNAKLL |
| | | | MDPKRQIFLDQNMLAVIDELMQALNENSETVPQKSSLEEPDE |
| | | | YKTKIKLCILLHAFRIRAVTIDRVMSYLNASGGGGGGSIWEL |
| | | | KKDVYVVELDWYPDAPGEMVVLTCDTPEEDGITWTLDQSSEV |
| | | | LGSGKTLTIQVKEFGDAGQYTCHKGGEVLSHSLLLLHKKEDG |
| | | | IWSTDILKDQKEPKNKTFLRCEAKNYSGRFTCWWLTTISTDL |
| | | | TFSVKSSRGSSDPQGVTCGAATLSAERVRGDNKEYEYSVECQ |
| | | | EDSACPAAEEIVSIKIMVDAVHKLKYENYTSSFFIRDIIKPD |
| | | | PPKNLQLKPLKNSRQVEVSWEYPDTWSTPHSYFSLTFCVQVQ |
| | | | GKSKREKKDRVFTDKTSATVICRKNASISVRAQDRYYSSSWS |
| | | | EWASVPCS |
| | Heavy Chain | 306 | See Table 19. |
| | (Hole) | | |
| | Light Chain | 245 | See Table 19. |
| BCA320 | Heavy Chain | 357 | EVRLVESGGGLVKPGGSLRLSCAASGFTFSNYYMSWIRQAPE |
| See e.g., | (Knob) | | KRLELVSAINSDGGITYYLDTVKGRFTISRDNAKNSLYLQMN |
| FIG. 2 | scIL-12 (IL- | | SLRAEDTALFYCARHRSGYFSMDYWGQGTSVTVSSASTKGPS |
| | 12p40 | | VFPLAPSSKSTSGGTAALGCLVKDYFPEPVTVSWNSGALTSG |
| | Variant G) | | VHTFPAVLQSSGLYSLSSVVTVPSSSLGTQTYICNVNHKPSN |
| | | | TKVDKKVEPKSCDKTHTCPPCPAPELLGGPSVFLFPPKPKDT |
| | | | LMISRTPEVTCVVVDVSHEDPEVKFNWYVDGVEVHNAKTKPR |
| | | | EEQYNSTYRVVSVLTVLHQDWLNGKEYKCKVSNKALPAPIEK |
| | | | TISKAKGQPREPQVYTLPPSRDELTKNQVSLWCLVKGFYPSD |
| | | | IAVEWESNGQPENNYKTTPPVLDSDGSFFLYSKLTVDKSRWQ |
| | | | QGNVFSCSVMHEALHNHYTQKSLSLSPGKGGGGSGGGGSGGG |
| | | | GSRNLPVATPDPGMFPCLHHSQNLLRAVSNMLQKARQTLEFY |
| | | | PCTSEEIDHEDITKDKTSTVEACLPLELTKNESCLNSRETSF |
| | | | ITNGSCLASRKTSFMMALCLSSIYEDLKMYQVEFKTMNAKLL |
| | | | MDPKRQIFLDQNMLAVIDELMQALNENSETVPQKSSLEEPDE |
| | | | YKTKIKLCILLHAFRIRAVTIDRVMSYLNASGGGGGGSIWEL |
| | | | KKDVYVVELDWYPDAPGEMVVLTCDTPEEDGITWTLDQSSEV |
| | | | LGSGKTLTIQVKEFGDAGQYTCHKGGEVLSHSLLLLHKKEDG |
| | | | IWSTDILKDQKEPKNKTFLRCEAKNYSGRFTCWWLTTISTDL |
| | | | TFSVKSSRGSSDPQGVTCGAATLSAERVRGDNKEYEYSVECQ |
| | | | EDSACPAAEEIQSIKGMVDAVHKLKYENYTSSFFIRDIIKPD |
| | | | PPKNLQLKPLKNSRQVEVSWEYPDTWSTPHSYFSLTFCVQVQ |
| | | | GKSKREKKDRVFTDKTSATVICRKNASISVRAQDRYYSSSWS |
| | | | EWASVPCS |
| | Heavy Chain | 306 | See Table 19. |
| | (Hole) | | |
| | Light Chain | 245 | See Table 19. |
| BCA321 | Heavy Chain | 358 | EVRLVESGGGLVKPGGSLRLSCAASGFTFSNYYMSWIRQAPE |
| See e.g., | (Knob) | | KRLELVSAINSDGGITYYLDTVKGRFTISRDNAKNSLYLQMN |
| FIG. 2 | scIL-12 (IL- | | SLRAEDTALFYCARHRSGYFSMDYWGQGTSVTVSSASTKGPS |
| | 12p40 | | VFPLAPSSKSTSGGTAALGCLVKDYFPEPVTVSWNSGALTSG |
| | Variant H) | | VHTFPAVLQSSGLYSLSSVVTVPSSSLGTQTYICNVNHKPSN |
| | | | TKVDKKVEPKSCDKTHTCPPCPAPELLGGPSVFLFPPKPKDT |
| | | | LMISRTPEVTCVVVDVSHEDPEVKFNWYVDGVEVHNAKTKPR |
| | | | EEQYNSTYRVVSVLTVLHQDWLNGKEYKCKVSNKALPAPIEK |
| | | | TISKAKGQPREPQVYTLPPSRDELTKNQVSLWCLVKGFYPSD |
| | | | IAVEWESNGQPENNYKTTPPVLDSDGSFFLYSKLTVDKSRWQ |
| | | | QGNVFSCSVMHEALHNHYTQKSLSLSPGKGGGGSGGGGSGGG |
| | | | GSRNLPVATPDPGMFPCLHHSQNLLRAVSNMLQKARQTLEFY |
| | | | PCTSEEIDHEDITKDKTSTVEACLPLELTKNESCLNSRETSE |
| | | | ITNGSCLASRKTSFMMALCLSSIYEDLKMYQVEFKTMNAKLL |
| | | | MDPKRQIFLDQNMLAVIDELMQALNENSETVPQKSSLEEPDE |
| | | | YKTKIKLCILLHAFRIRAVTIDRVMSYLNASGGGGGGSIWEL |
| | | | KKDVYVVELDWYPDAPGEMVVLTCDTPEEDGITWTLDQSSEV |
| | | | LGSGKTLTIQVKEFGDAGQYTCHKGGEVLSHSLLLLHKKEDG |
| | | | IWSTDILKDQKEPKNKTFLRCEAKNYSGRFTCWWLTTISTDL |
| | | | TFSVKSSRGSSDPQGVTCGAATLSAERVRGDNKEYEYSVECQ |
| | | | EDSACPAAEESLPIEVMVDAVHIKYENYTSSFFIRDIIKPDP |
| | | | PKNLQLKPLKNSRQVEVSWEYPDTWSTPHSYFSLTFCVQVQG |
| | | | KSKREKKDRVFTDKTSATVICRKNASISVRAQDRYYSSSWSE |
| | | | WASVPCS |
| | Heavy Chain | 306 | See Table 19. |
| | (Hole) | | |
| | Light Chain | 245 | See Table 19. |
| BCA322 | Heavy Chain | 359 | EVRLVESGGGLVKPGGSLRLSCAASGFTFSNYYMSWIRQAPE |
| See e.g., | (Knob) | | KRLELVSAINSDGGITYYLDTVKGRFTISRDNAKNSLYLQMN |
| FIG. 2 | scIL-12 (IL- | | SLRAEDTALFYCARHRSGYFSMDYWGQGTSVTVSSASTKGPS |
| | 12p40 | | VFPLAPSSKSTSGGTAALGCLVKDYFPEPVTVSWNSGALTSG |
| | Variant I) | | VHTFPAVLQSSGLYSLSSVVTVPSSSLGTQTYICNVNHKPSN |
| | | | TKVDKKVEPKSCDKTHTCPPCPAPELLGGPSVFLFPPKPKDT |
| | | | LMISRTPEVTCVVVDVSHEDPEVKFNWYVDGVEVHNAKTKPR |
| | | | EEQYNSTYRVVSVLTVLHQDWLNGKEYKCKVSNKALPAPIEK |
| | | | TISKAKGQPREPQVYTLPPSRDELTKNQVSLWCLVKGFYPSD |
| | | | IAVEWESNGQPENNYKTTPPVLDSDGSFFLYSKLTVDKSRWQ |
| | | | QGNVFSCSVMHEALHNHYTQKSLSLSPGKGGGGSGGGGSGGG |
| | | | GSRNLPVATPDPGMFPCLHHSQNLLRAVSNMLQKARQTLEFY |
| | | | PCTSEEIDHEDITKDKTSTVEACLPLELTKNESCLNSRETSE |
| | | | ITNGSCLASRKTSFMMALCLSSIYEDLKMYQVEFKTMNAKLL |
| | | | MDPKRQIFLDQNMLAVIDELMQALNENSETVPQKSSLEEPDE |
| | | | YKTKIKLCILLHAFRIRAVTIDRVMSYLNASGGGGGGSIWEL |
| | | | KKDVYVVELDWYPDAPGEMVVLTCDTPEEDGITWTLDQSSEV |
| | | | LGSGKTLTIQVKEFGDAGQYTCHKGGEVLSHSLLLLHKKEDG |
| | | | IWSTDILKDQKEPKNKTFLRCEAKNYSGRFTCWWLTTISTDL |
| | | | TFSVKSSRGSSDPQGVTCGAATLSAERVRGDNKEYEYSVECQ |
| | | | EDSACPAAEESLPIEVMVDAVHSITSANYTSSFFIRDIIKPD |
| | | | PPKNLQLKPLKNSRQVEVSWEYPDTWSTPHSYFSLTFCVQVQ |
| | | | GKSKREKKDRVFTDKTSATVICRKNASISVRAQDRYYSSSWS |
| | | | EWASVPCS |
| | Heavy Chain | 306 | See Table 19. |
| | (Hole) | | |
| | Light Chain | 245 | See Table 19. |
| BCA326 | Heavy Chain | 360 | EVRLVESGGGLVKPGGSLRLSCAASGFTFSNYYMSWIRQAPE |
| See e.g., | (Knob) | | KRLELVSAINSDGGITYYLDTVKGRFTISRDNAKNSLYLQMN |
| FIG. 2 | scIL-12 (IL- | | SLRAEDTALFYCARHRSGYFSMDYWGQGTSVTVSSASTKGPS |
| | 12p40 | | VFPLAPSSKSTSGGTAALGCLVKDYFPEPVTVSWNSGALTSG |
| | Variant J) | | VHTFPAVLQSSGLYSLSSVVTVPSSSLGTQTYICNVNHKPSN |
| | | | TKVDKKVEPKSCDKTHTCPPCPAPELLGGPSVFLFPPKPKDT |
| | | | LMISRTPEVTCVVVDVSHEDPEVKFNWYVDGVEVHNAKTKPR |
| | | | EEQYNSTYRVVSVLTVLHQDWLNGKEYKCKVSNKALPAPIEK |
| | | | TISKAKGQPREPQVYTLPPSRDELTKNQVSLWCLVKGFYPSD |
| | | | IAVEWESNGQPENNYKTTPPVLDSDGSFFLYSKLTVDKSRWQ |
| | | | QGNVFSCSVMHEALHNHYTQKSLSLSPGKGGGGSGGGGSGGG |
| | | | GSRNLPVATPDPGMFPCLHHSQNLLRAVSNMLQKARQTLEFY |
| | | | PCTSEEIDHEDITKDKTSTVEACLPLELTKNESCLNSRETSF |
| | | | ITNGSCLASRKTSEMMALCLSSIYEDLKMYQVEFKTMNAKLL |
| | | | MDPKRQIFLDQNMLAVIDELMQALNENSETVPQKSSLEEPDE |
| | | | YKTKIKLCILLHAFRIRAVTIDRVMSYLNASGGGGGGSIWEL |
| | | | KKDVYVVELDWYPDAPGEMVVLTCDTPEEDGITWTLDQSSEV |
| | | | LGSGKTLTIQVKAAGDAGQYTCHKGGEVLSHSLLLLHKKEDG |
| | | | IWSTDILKDQKEPKNKTFLRCEAKNYSGRFTCWWLTTISTDL |
| | | | TFSVKSSRGSSDPQGVTCGAATLSAERVRGDNKEYEYSVECQ |
| | | | EDSACPAAEESLPIEVMVDAVHKLKYENYTSSFFIRDIIKPD |
| | | | PPKNLQLKPLKNSRQVEVSWEYPDTWSTPHSYFSLTFCVQVQ |
| | | | GKSKREKKDRVFTDKTSATVICRKNASISVRAQDRYYSSSWS |
| | | | EWASVPCS |
| | Heavy Chain | 306 | See Table 19. |
| | (Hole) | | |
| | Light Chain | 245 | See Table 19. |
| BCA327 | Heavy Chain | 361 | EVRLVESGGGLVKPGGSLRLSCAASGFTFSNYYMSWIRQAPE |
| See e.g., | (Knob) | | KRLELVSAINSDGGITYYLDTVKGRFTISRDNAKNSLYLQMN |
| FIG. 2 | scIL-12 (IL- | | SLRAEDTALFYCARHRSGYFSMDYWGQGTSVTVSSASTKGPS |
| | 12p40 | | VFPLAPSSKSTSGGTAALGCLVKDYFPEPVTVSWNSGALTSG |
| | Variant A) | | VHTFPAVLQSSGLYSLSSVVTVPSSSLGTQTYICNVNHKPSN |
| | | | TKVDKKVEPKSCDKTHTCPPCPAPELLGGPSVFLFPPKPKDT |
| | | | LMISRTPEVTCVVVDVSHEDPEVKFNWYVDGVEVHNAKTKPR |
| | | | EEQYNSTYRVVSVLTVLHQDWLNGKEYKCKVSNKALPAPIEK |
| | | | TISKAKGQPREPQVYTLPPSRDELTKNQVSLWCLVKGFYPSD |
| | | | IAVEWESNGQPENNYKTTPPVLDSDGSFFLYSKLTVDKSRWQ |
| | | | QGNVFSCSVMHEALHNHYTQKSLSLSPGKGGGGSGGGGSGGG |
| | | | GSRNLPVATPDPGMFPCLHHSQNLLRAVSNMLQKARQTLEFY |
| | | | PCTSEEIDHEDITKDKTSTVEACLPLELTKNESCLNSRETSF |
| | | | ITNGSCLASRKTSFMMALCLSSIYEDLKMYQVEFKTMNAKLI |
| | | | MDPKRQIFLDQNMLAVIDELMQALNENSETVPQKSSLEEPDE |
| | | | YKTKIKLCILLHAFRIRAVTIDRVMSYLNASGGGGGGSIWEL |
| | | | KKDVYVVELDWYPDAPGEMVVLTCDTPEEDGITWTLDQSSEV |
| | | | LGSGKTLTIQVKAAGDAGQYTCHKGGEVLSHSLLLLHAKEDG |
| | | | IWSTDILKDQKEPKNKTFLRCEAKNYSGRFTCWWLTTISTDL |
| | | | TFSVKSSRGSSDPQGVTCGAATLSAERVRGDNKEYEYSVECQ |
| | | | EDSACPAAEESLPIEVMVDAVHKLKYENYTSSFFIRDIIKPD |
| | | | PPKNLQLKPLKNSRQVEVSWEYPDTWSTPHSYFSLTFCVQVQ |
| | | | GKSKREKKDRVFTDKTSATVICRKNASISVRAQDRYYSSSWS |
| | | | EWASVPCS |
| | Heavy Chain | 306 | See Table 19. |
| | (Hole) | | |
| | Light Chain | 245 | See Table 19. |
| BCA328 | Heavy Chain | 362 | EVRLVESGGGLVKPGGSLRLSCAASGFTFSNYYMSWIRQAPE |
| See e.g., | (Knob) | | KRLELVSAINSDGGITYYLDTVKGRFTISRDNAKNSLYLQMN |
| FIG. 2 | scIL-12 (IL- | | SLRAEDTALFYCARHRSGYFSMDYWGQGTSVTVSSASTKGPS |
| | 12p40 | | VFPLAPSSKSTSGGTAALGCLVKDYFPEPVTVSWNSGALTSG |
| | Variant L) | | VHTFPAVLQSSGLYSLSSVVTVPSSSLGTQTYICNVNHKPSN |
| | | | TKVDKKVEPKSCDKTHTCPPCPAPELLGGPSVFLFPPKPKDT |
| | | | LMISRTPEVTCVVVDVSHEDPEVKFNWYVDGVEVHNAKTKPR |
| | | | EEQYNSTYRVVSVLTVLHQDWLNGKEYKCKVSNKALPAPIEK |
| | | | TISKAKGQPREPQVYTLPPSRDELTKNQVSLWCLVKGFYPSD |
| | | | IAVEWESNGQPENNYKTTPPVLDSDGSFFLYSKLTVDKSRWQ |
| | | | QGNVFSCSVMHEALHNHYTQKSLSLSPGKGGGGSGGGGSGGG |
| | | | GSRNLPVATPDPGMFPCLHHSQNLLRAVSNMLQKARQTLEFY |
| | | | PCTSEEIDHEDITKDKTSTVEACLPLELTKNESCLNSRETSE |
| | | | ITNGSCLASRKTSEMMALCLSSIYEDLKMYQVEFKTMNAKLL |
| | | | MDPKRQIFLDQNMLAVIDELMQALNENSETVPQKSSLEEPDE |
| | | | YKTKIKLCILLHAFRIRAVTIDRVMSYLNASGGGGGGSIWEL |
| | | | KKDVYVVELDWYPDAPGEMVVLTCDTPEEDGITWTLDQSSEV |
| | | | LGSGKTLTIQVKAAGDAGQYTCHKGGEVLSHSLLLLHAKEDG |
| | | | IWSTDILKDQKEPKNKTFLRCEAKNYSGRFTCWWLTTISTDL |
| | | | TFSVKSSRGSSDPQGVTCGAATLSAERVRGDNKEYEYSVECQ |
| | | | EDSACPAAEESLPIEVMVDAVHALKYENYTSSFFIRDIIKPD |
| | | | PPKNLQLKPLKNSRQVEVSWEYPDTWSTPHSYFSLTFCVQVQ |
| | | | GKSKREKKDRVFTDKTSATVICRKNASISVRAQDRYYSSSWS |
| | | | EWASVPCS |
| | Heavy Chain | 306 | See Table 19. |
| | (Hole) | | |
| | Light Chain | 245 | See Table 19. |
| BCA329 | Heavy Chain | 363 | EVRLVESGGGLVKPGGSLRLSCAASGFTFSNYYMSWIRQAPE |
| See e.g., | (Knob) | | KRLELVSAINSDGGITYYLDTVKGRFTISRDNAKNSLYLQMN |
| FIG. 2 | scIL-12 (IL- | | SLRAEDTALFYCARHRSGYFSMDYWGQGTSVTVSSASTKGPS |
| | 12p40 variant | | VFPLAPSSKSTSGGTAALGCLVKDYFPEPVTVSWNSGALTSG |
| | M) | | VHTFPAVLQSSGLYSLSSVVTVPSSSLGTQTYICNVNHKPSN |
| | | | TKVDKKVEPKSCDKTHTCPPCPAPELLGGPSVFLFPPKPKDT |
| | | | LMISRTPEVTCVVVDVSHEDPEVKFNWYVDGVEVHNAKTKPR |
| | | | EEQYNSTYRVVSVLTVLHQDWLNGKEYKCKVSNKALPAPIEK |
| | | | TISKAKGQPREPQVYTLPPSRDELTKNQVSLWCLVKGFYPSD |
| | | | IAVEWESNGQPENNYKTTPPVLDSDGSFFLYSKLTVDKSRWQ |
| | | | QGNVFSCSVMHEALHNHYTQKSLSLSPGKGGGGSGGGGSGGG |
| | | | GS |
| | | | RNLPVATPDPGMFPCLHHSQNLLRAVSNMLQKARQTLEFYPC |
| | | | TSEEIDHEDITKDKTSTVEACLPLELTKNESCLNSRETSFIT |
| | | | NGSCLASRKTSEMMALCLSSIYEDLKMYQVEFKTMNAKLLMD |
| | | | PKRQIFLDQNMLAVIDELMQALNENSETVPQKSSLEEPDFYK |
| | | | TKIKLCILLHAFRIRAVTIDRVMSYLNASGGGGGGSIWELKK |
| | | | DVYVVELDWYAAAPGEMVVLTCDTPEEDGITWTLDQSSEVLG |
| | | | SGKTLTIQVKAAGDAGQYTCHKGGEVLSHSLLLLHKKEDGIW |
| | | | STDILKDQKEPKNKTFLRCEAKNYSGRFTCWWLTTISTDLTF |
| | | | SVKSSRGSSDPQGVTCGAATLSAERVRGDNKEYEYSVECQED |
| | | | SACPAAEESLPIEVMVDAVHKLKYENYTSSFFIRDIIKPDPP |
| | | | KNLQLKPLKNSRQVEVSWEYPDTWSTPHSYFSLTFCVQVQGK |
| | | | SKREKKDRVFTDKTSATVICRKNASISVRAQDRYYSSSWSEW |
| | | | ASVPCS |
| | Heavy Chain | 306 | See Table 19. |
| | (Hole) | | |
| | Light Chain | 245 | See Table 19. |
| BCA330 | Heavy Chain | 364 | EVRLVESGGGLVKPGGSLRLSCAASGFTFSNYYMSWIRQAPE |
| See e.g., | (Knob) | | KRLELVSAINSDGGITYYLDTVKGRFTISRDNAKNSLYLQMN |
| FIG. 2 | scIL-12 (IL- | | SLRAEDTALFYCARHRSGYFSMDYWGQGTSVTVSSASTKGPS |
| | 12p40 variant | | VFPLAPSSKSTSGGTAALGCLVKDYFPEPVTVSWNSGALTSG |
| | N) | | VHTFPAVLQSSGLYSLSSVVTVPSSSLGTQTYICNVNHKPSN |
| | | | TKVDKKVEPKSCDKTHTCPPCPAPELLGGPSVFLFPPKPKDT |
| | | | LMISRTPEVTCVVVDVSHEDPEVKFNWYVDGVEVHNAKTKPR |
| | | | EEQYNSTYRVVSVLTVLHQDWLNGKEYKCKVSNKALPAPIEK |
| | | | TISKAKGQPREPQVYTLPPSRDELTKNQVSLWCLVKGFYPSD |
| | | | IAVEWESNGQPENNYKTTPPVLDSDGSFFLYSKLTVDKSRWQ |
| | | | QGNVFSCSVMHEALHNHYTQKSLSLSPGKGGGGSGGGGSGGG |
| | | | GSRNLPVATPDPGMFPCLHHSQNLLRAVSNMLQKARQTLEFY |
| | | | PCTSEEIDHEDITKDKTSTVEACLPLELTKNESCLNSRETSF |
| | | | ITNGSCLASRKTSFMMALCLSSIYEDLKMYQVEFKTMNAKLL |
| | | | MDPKRQIFLDQNMLAVIDELMQALNENSETVPQKSSLEEPDE |
| | | | YKTKIKLCILLHAFRIRAVTIDRVMSYLNASGGGGGGSIWEL |
| | | | KKDVYVVELDAYPDAPGEMVVLTCDTPEEDGITWTLDQSSEV |
| | | | LGSGKTLTIQVKEAGDAGQYTCHKGGEVLSHSLLLLHKKEDG |
| | | | IWSTDILKDQKEPKNKTFLRCEAKNYSGRFTCWWLTTISTDL |
| | | | TFSVKSSRGSSDPQGVTCGAATLSAERVRGDNKEYEYSVECQ |
| | | | EDSACPAAEESLPIEVMVDAVHALKYENYTSSFFIRDIIKPD |
| | | | PPKNLQLKPLKNSRQVEVSWEYPDTWSTPHSYFSLTFCVQVQ |
| | | | GKSKREKKDRVFTDKTSATVICRKNASISVRAQDRYYSSSWS |
| | | | EWASVPCS |
| | Heavy Chain | 306 | See Table 19. |
| | (Hole) | | |
| | Light Chain | 245 | See Table 19. |
| BCA331 | Heavy Chain | 365 | EVRLVESGGGLVKPGGSLRLSCAASGFTFSNYYMSWIRQAPE |
| See e.g., | (Knob) | | KRLELVSAINSDGGITYYLDTVKGRFTISRDNAKNSLYLQMN |
| FIG. 2 | scIL-12 (IL- | | SLRAEDTALFYCARHRSGYFSMDYWGQGTSVTVSSASTKGPS |
| | 12p40 variant | | VFPLAPSSKSTSGGTAALGCLVKDYFPEPVTVSWNSGALTSG |
| | O) | | VHTFPAVLQSSGLYSLSSVVTVPSSSLGTQTYICNVNHKPSN |
| | | | TKVDKKVEPKSCDKTHTCPPCPAPELLGGPSVFLFPPKPKDT |
| | | | LMISRTPEVTCVVVDVSHEDPEVKFNWYVDGVEVHNAKTKPR |
| | | | EEQYNSTYRVVSVLTVLHQDWLNGKEYKCKVSNKALPAPIEK |
| | | | TISKAKGQPREPQVYTLPPSRDELTKNQVSLWCLVKGFYPSD |
| | | | IAVEWESNGQPENNYKTTPPVLDSDGSFFLYSKLTVDKSRWQ |
| | | | QGNVFSCSVMHEALHNHYTQKSLSLSPGKGGGGSGGGGSGGG |
| | | | GSRNLPVATPDPGMFPCLHHSQNLLRAVSNMLQKARQTLEFY |
| | | | PCTSEEIDHEDITKDKTSTVEACLPLELTKNESCLNSRETSE |
| | | | ITNGSCLASRKTSFMMALCLSSIYEDLKMYQVEFKTMNAKLL |
| | | | MDPKRQIFLDQNMLAVIDELMQALNENSETVPQKSSLEEPDE |
| | | | YKTKIKLCILLHAFRIRAVTIDRVMSYLNASGGGGGGSIWEL |
| | | | KKDVYVVELDAYPDAPGEMVVLTCDTPEEDGITWTLDQSSEV |
| | | | LGSGKTLTIQVKEAGDAGQYTCHKGGEVLSHSLLLLHKKEDG |
| | | | IWSTDILKDQKEPKNKTFLRCEAKNYSGRFTCWWLTTISTDL |
| | | | TFSVKSSRGSSDPQGVTCGAATLSAERVRGDNKEYEYSVECQ |
| | | | EDSACPAAEESLPIEVMVDAVHKLAYENYTSSFFIRDIIKPD |
| | | | PPKNLQLKPLKNSRQVEVSWEYPDTWSTPHSYFSLTFCVQVQ |
| | | | GKSKREKKDRVFTDKTSATVICRKNASISVRAQDRYYSSSWS |
| | | | EWASVPCS |
| | Heavy Chain | 306 | See Table 19. |
| | (Hole) | | |
| | Light Chain | 245 | See Table 19. |
| BCA332 | Heavy Chain | 366 | EVRLVESGGGLVKPGGSLRLSCAASGFTFSNYYMSWIRQAPE |
| See e.g., | (Knob) | | KRLELVSAINSDGGITYYLDTVKGRFTISRDNAKNSLYLQMN |
| FIG. 2 | scIL-12 (IL- | | SLRAEDTALFYCARHRSGYFSMDYWGQGTSVTVSSASTKGPS |
| | 12p40 variant | | VFPLAPSSKSTSGGTAALGCLVKDYFPEPVTVSWNSGALTSG |
| | P) | | VHTFPAVLQSSGLYSLSSVVTVPSSSLGTQTYICNVNHKPSN |
| | | | TKVDKKVEPKSCDKTHTCPPCPAPELLGGPSVFLFPPKPKDT |
| | | | LMISRTPEVTCVVVDVSHEDPEVKFNWYVDGVEVHNAKTKPR |
| | | | EEQYNSTYRVVSVLTVLHQDWLNGKEYKCKVSNKALPAPIEK |
| | | | TISKAKGQPREPQVYTLPPSRDELTKNQVSLWCLVKGFYPSD |
| | | | IAVEWESNGQPENNYKTTPPVLDSDGSFFLYSKLTVDKSRWQ |
| | | | QGNVFSCSVMHEALHNHYTQKSLSLSPGKGGGGSGGGGSGGG |
| | | | GSRNLPVATPDPGMFPCLHHSQNLLRAVSNMLQKARQTLEFY |
| | | | PCTSEEIDHEDITKDKTSTVEACLPLELTKNESCLNSRETSF |
| | | | ITNGSCLASRKTSFMMALCLSSIYEDLKMYQVEFKTMNAKLL |
| | | | MDPKRQIFLDQNMLAVIDELMQALNENSETVPQKSSLEEPDE |
| | | | YKTKIKLCILLHAFRIRAVTIDRVMSYLNASGGGGGGSIWEL |
| | | | KKDVYVVELDWYPDAPGEMVVLTCDTPEEDGITWTLDQSSEV |
| | | | LGSGKTLTIQVKEFGDAGQYTCHKGGEVLSHSLLLLHAKEDG |
| | | | IWSTDILKDQKEPKNKTFLRCEAKNYSGRFTCWWLTTISTDL |
| | | | TFSVKSSRGSSDPQGVTCGAATLSAERVRGDNKEYEYSVECQ |
| | | | EDSACPAAEESLPIEVMVDAVHALAYENYTSSFFIRDIIKPD |
| | | | PPKNLQLKPLKNSRQVEVSWEYPDTWSTPHSYFSLTFCVQVQ |
| | | | GKSKREKKDRVFTDKTSATVICRKNASISVRAQDRYYSSSWS |
| | | | EWASVPCS |
| | Heavy Chain | 306 | See Table 19. |
| | (Hole) | | |
| | Light Chain | 245 | See Table 19. |
| BCA333.16 | Heavy Chain | 367 | EVRLVESGGGLVKPGGSLRLSCAASGFTFSNYYMSWIRQAPE |
| See e.g., | (Knob) | | KRLELVSAINSDGGITYYLDTVKGRFTISRDNAKNSLYLQMN |
| FIG. 2 | scIL-12 (IL- | | SLRAEDTALFYCARHRSGYFSMDYWGQGTSVTVSSASTKGPS |
| | 12p40 variant | | VFPLAPSSKSTSGGTAALGCLVKDYFPEPVTVSWNSGALTSG |
| | Q) | | VHTFPAVLQSSGLYSLSSVVTVPSSSLGTQTYICNVNHKPSN |
| | | | TKVDKKVEPKSCDKTHTCPPCPAPELLGGPSVFLFPPKPKDT |
| | | | LMISRTPEVTCVVVDVSHEDPEVKFNWYVDGVEVHNAKTKPR |
| | | | EEQYNSTYRVVSVLTVLHQDWLNGKEYKCKVSNKALPAPIEK |
| | | | TISKAKGQPREPQVYTLPPSRDELTKNQVSLWCLVKGFYPSD |
| | | | IAVEWESNGQPENNYKTTPPVLDSDGSFFLYSKLTVDKSRWQ |
| | | | QGNVFSCSVMHEALHNHYTQKSLSLSPGKGGGGSGGGGSGGG |
| | | | GSRNLPVATPDPGMFPCLHHSQNLLRAVSNMLQKARQTLEFY |
| | | | PCTSEEIDHEDITKDKTSTVEACLPLELTKNESCLNSRETSE |
| | | | ITNGSCLASRKTSEMMALCLSSIYEDLKMYQVEFKTMNAKLL |
| | | | MDPKRQIFLDQNMLAVIDELMQALNENSETVPQKSSLEEPDE |
| | | | YKTKIKLCILLHAFRIRAVTIDRVMSYLNASGGGGGGSIWEL |
| | | | KKDVYVVELDAYPDAPGEMVVLTCDTPEEDGITWTLDQSSEV |
| | | | LGSGKTLTIQVKEAGDAGQYTCHKGGEVLSHSLLLLHAKEDG |
| | | | IWSTDILKDQKEPKNKTFLRCEAKNYSGRFTCWWLTTISTDL |
| | | | TFSVKSSRGSSDPQGVTCGAATLSAERVRGDNKEYEYSVECQ |
| | | | EDSACPAAEESLPIEVMVDAVHKLAYENYTSSFFIRDIIKPD |
| | | | PPKNLQLKPLKNSRQVEVSWEYPDTWSTPHSYFSLTFCVQVQ |
| | | | GKSKREKKDRVFTDKTSATVICRKNASISVRAQDRYYSSSWS |
| | | | EWASVPCS |
| | Heavy Chain | 306 | See Table 19. |
| | (Hole) | | |
| | Light Chain | 245 | See Table 19. |
| BCA334 | Heavy Chain | 368 | EVRLVESGGGLVKPGGSLRLSCAASGFTFSNYYMSWIRQAPE |
| See e.g., | (Knob) | | KRLELVSAINSDGGITYYLDTVKGRFTISRDNAKNSLYLQMN |
| FIG. 2 | scIL-12 (IL- | | SLRAEDTALFYCARHRSGYFSMDYWGQGTSVTVSSASTKGPS |
| | 12p40 variant | | VFPLAPSSKSTSGGTAALGCLVKDYFPEPVTVSWNSGALTSG |
| | R) | | VHTFPAVLQSSGLYSLSSVVTVPSSSLGTQTYICNVNHKPSN |
| | | | TKVDKKVEPKSCDKTHTCPPCPAPELLGGPSVFLFPPKPKDT |
| | | | LMISRTPEVTCVVVDVSHEDPEVKFNWYVDGVEVHNAKTKPR |
| | | | EEQYNSTYRVVSVLTVLHQDWLNGKEYKCKVSNKALPAPIEK |
| | | | TISKAKGQPREPQVYTLPPSRDELTKNQVSLWCLVKGFYPSD |
| | | | IAVEWESNGQPENNYKTTPPVLDSDGSFFLYSKLTVDKSRWQ |
| | | | QGNVFSCSVMHEALHNHYTQKSLSLSPGKGGGGSGGGGSGGG |
| | | | GSRNLPVATPDPGMFPCLHHSQNLLRAVSNMLQKARQTLEFY |
| | | | PCTSEEIDHEDITKDKTSTVEACLPLELTKNESCLNSRETSF |
| | | | ITNGSCLASRKTSFMMALCLSSIYEDLKMYQVEFKTMNAKLL |
| | | | MDPKRQIFLDQNMLAVIDELMQALNENSETVPQKSSLEEPDE |
| | | | YKTKIKLCILLHAFRIRAVTIDRVMSYLNASGGGGGGSIWEL |
| | | | KKDVYVVELDWYPDAPGEMVVLTCDTPEEDGITWTLDQSSEV |
| | | | LGSGKTLTIQVKEFGDAGQYTCHKGGEVLSHSLLLLHKKEDG |
| | | | IWSTDILKDQKEPKNKTFLRCEAKNYSGRFTCWWLTTISTDL |
| | | | TFSVKSSRGSSDPQGVTCGAATLSAERVRGDNKEYEYSVECQ |
| | | | EDSACPAAEESLPIEVMVDAVAALAYENYTSSFFIRDIIKPD |
| | | | PPKNLQLKPLKNSRQVEVSWEYPDTWSTPHSYFSLTFCVQVQ |
| | | | GKSKREKKDRVFTDKTSATVICRKNASISVRAQDRYYSSSWS |
| | | | EWASVPCS |
| | Heavy Chain | 306 | See Table 19. |
| | (Hole) | | |
| | Light Chain | 245 | See Table 19. |

In some embodiments, the anti-CAIX antibody comprises a first light chain comprising an amino acid at least 90%, 91%, 92%, 93%, 94%, 95%, 96%, 97%, 98%, 99%, or 100% identical to the amino acid sequence of SEQ ID NO: 245; a first heavy chain comprising an amino acid at least 90%, 91%, 92%, 93%, 94%, 95%, 96%, 97%, 98%, 99%, or 100% identical to the amino acid sequence of SEQ ID NO: 304; a second heavy chain comprising an amino acid at least 90%, 91%, 92%, 93%, 94%, 95%, 96%, 97%, 98%, 99%, or 100% identical to the amino acid sequence of SEQ ID NO: 305; and a second light chain comprising an amino acid at least 90%, 91%, 92%, 93%, 94%, 95%, 96%, 97%, 98%, 99%, or 100% identical to the amino acid sequence of SEQ ID NO: 245.

In some embodiments, the anti-CAIX antibody comprises a first light chain comprising an amino acid at least 90%, 91%, 92%, 93%, 94%, 95%, 96%, 97%, 98%, 99%, or 100% identical to the amino acid sequence of SEQ ID NO: 245; a first heavy chain comprising an amino acid at least 90%, 91%, 92%, 93%, 94%, 95%, 96%, 97%, 98%, 99%, or 100% identical to the amino acid sequence of SEQ ID NO: 304; a second heavy chain comprising an amino acid at least 90%, 91%, 92%, 93%, 94%, 95%, 96%, 97%, 98%, 99%, or 100% identical to the amino acid sequence of SEQ ID NO: 307; and a second light chain comprising an amino acid at least 90%, 91%, 92%, 93%, 94%, 95%, 96%, 97%, 98%, 99%, or 100% identical to the amino acid sequence of SEQ ID NO: 245.

In some embodiments, the anti-CAIX antibody comprises a first light chain comprising an amino acid at least 90%, 91%, 92%, 93%, 94%, 95%, 96%, 97%, 98%, 99%, or 100% identical to the amino acid sequence of SEQ ID NO: 245; a first heavy chain comprising an amino acid at least 90%, 91%, 92%, 93%, 94%, 95%, 96%, 97%, 98%, 99%, or 100% identical to the amino acid sequence of SEQ ID NO: 304; a second heavy chain comprising an amino acid at least 90%, 91%, 92%, 93%, 94%, 95%, 96%, 97%, 98%, 99%, or 100% identical to the amino acid sequence of SEQ ID NO: 308; and a second light chain comprising an amino acid at least 90%, 91%, 92%, 93%, 94%, 95%, 96%, 97%, 98%, 99%, or 100% identical to the amino acid sequence of SEQ ID NO: 245.

In some embodiments, the anti-CAIX antibody comprises a first light chain comprising an amino acid at least 90%, 91%, 92%, 93%, 94%, 95%, 96%, 97%, 98%, 99%, or 100% identical to the amino acid sequence of SEQ ID NO: 245; a first heavy chain comprising an amino acid at least 90%, 91%, 92%, 93%, 94%, 95%, 96%, 97%, 98%, 99%, or 100% identical to the amino acid sequence of SEQ ID NO: 304; a second heavy chain comprising an amino acid at least 90%, 91%, 92%, 93%, 94%, 95%, 96%, 97%, 98%, 99%, or 100% identical to the amino acid sequence of SEQ ID NO: 309; and a second light chain comprising an amino acid at least 90%, 91%, 92%, 93%, 94%, 95%, 96%, 97%, 98%, 99%, or 100% identical to the amino acid sequence of SEQ ID NO: 245.

In some embodiments, the anti-CAIX antibody comprises a first light chain comprising an amino acid at least 90%, 91%, 92%, 93%, 94%, 95%, 96%, 97%, 98%, 99%, or 100% identical to the amino acid sequence of SEQ ID NO: 245; a first heavy chain comprising an amino acid at least 90%, 91%, 92%, 93%, 94%, 95%, 96%, 97%, 98%, 99%, or 100% identical to the amino acid sequence of SEQ ID NO: 304; a second heavy chain comprising an amino acid at least 90%, 91%, 92%, 93%, 94%, 95%, 96%, 97%, 98%, 99%, or 100% identical to the amino acid sequence of SEQ ID NO: 310; and a second light chain comprising an amino acid at least 90%, 91%, 92%, 93%, 94%, 95%, 96%, 97%, 98%, 99%, or 100% identical to the amino acid sequence of SEQ ID NO: 245.

In some embodiments, the anti-CAIX antibody comprises a first light chain comprising an amino acid at least 90%, 91%, 92%, 93%, 94%, 95%, 96%, 97%, 98%, 99%, or 100% identical to the amino acid sequence of SEQ ID NO: 245; a first heavy chain comprising an amino acid at least 90%, 91%, 92%, 93%, 94%, 95%, 96%, 97%, 98%, 99%, or 100% identical to the amino acid sequence of SEQ ID NO: 304; a second heavy chain comprising an amino acid at least 90%, 91%, 92%, 93%, 94%, 95%, 96%, 97%, 98%, 99%, or 100% identical to the amino acid sequence of SEQ ID NO: 311; and a second light chain comprising an amino acid at least 90%, 91%, 92%, 93%, 94%, 95%, 96%, 97%, 98%, 99%, or 100% identical to the amino acid sequence of SEQ ID NO: 245.

In some embodiments, the anti-CAIX antibody comprises a first light chain comprising an amino acid at least 90%, 91%, 92%, 93%, 94%, 95%, 96%, 97%, 98%, 99%, or 100% identical to the amino acid sequence of SEQ ID NO: 245; a first heavy chain comprising an amino acid at least 90%, 91%, 92%, 93%, 94%, 95%, 96%, 97%, 98%, 99%, or 100% identical to the amino acid sequence of SEQ ID NO: 305; a second heavy chain comprising an amino acid at least 90%, 91%, 92%, 93%, 94%, 95%, 96%, 97%, 98%, 99%, or 100% identical to the amino acid sequence of SEQ ID NO: 312; and a second light chain comprising an amino acid at least 90%, 91%, 92%, 93%, 94%, 95%, 96%, 97%, 98%, 99%, or 100% identical to the amino acid sequence of SEQ ID NO: 245.

In some embodiments, the anti-CAIX antibody comprises a first light chain comprising an amino acid at least 90%, 91%, 92%, 93%, 94%, 95%, 96%, 97%, 98%, 99%, or 100% identical to the amino acid sequence of SEQ ID NO: 245; a first heavy chain comprising an amino acid at least 90%, 91%, 92%, 93%, 94%, 95%, 96%, 97%, 98%, 99%, or 100% identical to the amino acid sequence of SEQ ID NO: 305; a second heavy chain comprising an amino acid at least 90%, 91%, 92%, 93%, 94%, 95%, 96%, 97%, 98%, 99%, or 100% identical to the amino acid sequence of SEQ ID NO: 313; and a second light chain comprising an amino acid at least 90%, 91%, 92%, 93%, 94%, 95%, 96%, 97%, 98%, 99%, or 100% identical to the amino acid sequence of SEQ ID NO: 245.

In some embodiments, the anti-CAIX antibody comprises a first light chain comprising an amino acid at least 90%, 91%, 92%, 93%, 94%, 95%, 96%, 97%, 98%, 99%, or 100% identical to the amino acid sequence of SEQ ID NO: 245; a first heavy chain comprising an amino acid at least 90%, 91%, 92%, 93%, 94%, 95%, 96%, 97%, 98%, 99%, or 100% identical to the amino acid sequence of SEQ ID NO: 305; a second heavy chain comprising an amino acid at least 90%, 91%, 92%, 93%, 94%, 95%, 96%, 97%, 98%, 99%, or 100% identical to the amino acid sequence of SEQ ID NO: 314; and a second light chain comprising an amino acid at least 90%, 91%, 92%, 93%, 94%, 95%, 96%, 97%, 98%, 99%, or 100% identical to the amino acid sequence of SEQ ID NO: 245.

In some embodiments, the anti-CAIX antibody comprises a first light chain comprising an amino acid at least 90%, 91%, 92%, 93%, 94%, 95%, 96%, 97%, 98%, 99%, or 100% identical to the amino acid sequence of SEQ ID NO: 245; a first heavy chain comprising an amino acid at least 90%, 91%, 92%, 93%, 94%, 95%, 96%, 97%, 98%, 99%, or 100% identical to the amino acid sequence of SEQ ID NO: 305; a second heavy chain comprising an amino acid at least 90%, 91%, 92%, 93%, 94%, 95%, 96%, 97%, 98%, 99%, or 100% identical to the amino acid sequence of SEQ ID NO: 315; and a second light chain comprising an amino acid at least 90%, 91%, 92%, 93%, 94%, 95%, 96%, 97%, 98%, 99%, or 100% identical to the amino acid sequence of SEQ ID NO: 245.

In some embodiments, the anti-CAIX antibody comprises a first light chain comprising an amino acid at least 90%, 91%, 92%, 93%, 94%, 95%, 96%, 97%, 98%, 99%, or 100% identical to the amino acid sequence of SEQ ID NO: 245; a first heavy chain comprising an amino acid at least 90%, 91%, 92%, 93%, 94%, 95%, 96%, 97%, 98%, 99%, or 100% identical to the amino acid sequence of SEQ ID NO: 305; a second heavy chain comprising an amino acid at least 90%, 91%, 92%, 93%, 94%, 95%, 96%, 97%, 98%, 99%, or 100% identical to the amino acid sequence of SEQ ID NO: 316; and a second light chain comprising an amino acid at least 90%, 91%, 92%, 93%, 94%, 95%, 96%, 97%, 98%, 99%, or 100% identical to the amino acid sequence of SEQ ID NO: 245.

In some embodiments, the anti-CAIX antibody comprises a first light chain comprising an amino acid at least 90%, 91%, 92%, 93%, 94%, 95%, 96%, 97%, 98%, 99%, or 100% identical to the amino acid sequence of SEQ ID NO: 245; a first heavy chain comprising an amino acid at least 90%, 91%, 92%, 93%, 94%, 95%, 96%, 97%, 98%, 99%, or 100% identical to the amino acid sequence of SEQ ID NO: 305; a second heavy chain comprising an amino acid at least 90%, 91%, 92%, 93%, 94%, 95%, 96%, 97%, 98%, 99%, or 100% identical to the amino acid sequence of SEQ ID NO: 317; and a second light chain comprising an amino acid at least 90%, 91%, 92%, 93%, 94%, 95%, 96%, 97%, 98%, 99%, or 100% identical to the amino acid sequence of SEQ ID NO: 245.

In some embodiments, the anti-CAIX antibody comprises a first light chain comprising an amino acid at least 90%, 91%, 92%, 93%, 94%, 95%, 96%, 97%, 98%, 99%, or 100% identical to the amino acid sequence of SEQ ID NO: 245; a first heavy chain comprising an amino acid at least 90%, 91%, 92%, 93%, 94%, 95%, 96%, 97%, 98%, 99%, or 100% identical to the amino acid sequence of SEQ ID NO: 305; a second heavy chain comprising an amino acid at least 90%, 91%, 92%, 93%, 94%, 95%, 96%, 97%, 98%, 99%, or 100% identical to the amino acid sequence of SEQ ID NO: 318; and a second light chain comprising an amino acid at least 90%, 91%, 92%, 93%, 94%, 95%, 96%, 97%, 98%, 99%, or 100% identical to the amino acid sequence of SEQ ID NO: 245.

In some embodiments, the anti-CAIX antibody comprises a first light chain comprising an amino acid at least 90%, 91%, 92%, 93%, 94%, 95%, 96%, 97%, 98%, 99%, or 100% identical to the amino acid sequence of SEQ ID NO: 245; a first heavy chain comprising an amino acid at least 90%, 91%, 92%, 93%, 94%, 95%, 96%, 97%, 98%, 99%, or 100% identical to the amino acid sequence of SEQ ID NO: 305; a second heavy chain comprising an amino acid at least 90%, 91%, 92%, 93%, 94%, 95%, 96%, 97%, 98%, 99%, or 100% identical to the amino acid sequence of SEQ ID NO: 319; and a second light chain comprising an amino acid at least 90%, 91%, 92%, 93%, 94%, 95%, 96%, 97%, 98%, 99%, or 100% identical to the amino acid sequence of SEQ ID NO: 245.

In some embodiments, the anti-CAIX antibody comprises a first light chain comprising an amino acid at least 90%, 91%, 92%, 93%, 94%, 95%, 96%, 97%, 98%, 99%, or 100% identical to the amino acid sequence of SEQ ID NO: 245; a first heavy chain comprising an amino acid at least 90%, 91%, 92%, 93%, 94%, 95%, 96%, 97%, 98%, 99%, or 100% identical to the amino acid sequence of SEQ ID NO: 305; a second heavy chain comprising an amino acid at least 90%, 91%, 92%, 93%, 94%, 95%, 96%, 97%, 98%, 99%, or 100% identical to the amino acid sequence of SEQ ID NO: 320; and a second light chain comprising an amino acid at least 90%, 91%, 92%, 93%, 94%, 95%, 96%, 97%, 98%, 99%, or 100% identical to the amino acid sequence of SEQ ID NO: 245.

In some embodiments, the anti-CAIX antibody comprises a first light chain comprising an amino acid at least 90%, 91%, 92%, 93%, 94%, 95%, 96%, 97%, 98%, 99%, or 100% identical to the amino acid sequence of SEQ ID NO: 245; a first heavy chain comprising an amino acid at least 90%, 91%, 92%, 93%, 94%, 95%, 96%, 97%, 98%, 99%, or 100% identical to the amino acid sequence of SEQ ID NO: 305; a second heavy chain comprising an amino acid at least 90%, 91%, 92%, 93%, 94%, 95%, 96%, 97%, 98%, 99%, or 100% identical to the amino acid sequence of SEQ ID NO: 321; and a second light chain comprising an amino acid at least 90%, 91%, 92%, 93%, 94%, 95%, 96%, 97%, 98%, 99%, or 100% identical to the amino acid sequence of SEQ ID NO: 245.

In some embodiments, the anti-CAIX antibody comprises a first light chain comprising an amino acid at least 90%, 91%, 92%, 93%, 94%, 95%, 96%, 97%, 98%, 99%, or 100% identical to the amino acid sequence of SEQ ID NO: 245; a first heavy chain comprising an amino acid at least 90%, 91%, 92%, 93%, 94%, 95%, 96%, 97%, 98%, 99%, or 100% identical to the amino acid sequence of SEQ ID NO: 305; a second heavy chain comprising an amino acid at least 90%, 91%, 92%, 93%, 94%, 95%, 96%, 97%, 98%, 99%, or 100% identical to the amino acid sequence of SEQ ID NO: 322; and a second light chain comprising an amino acid at least 90%, 91%, 92%, 93%, 94%, 95%, 96%, 97%, 98%, 99%, or 100% identical to the amino acid sequence of SEQ ID NO: 245.

In some embodiments, the anti-CAIX antibody comprises a first light chain comprising an amino acid at least 90%, 91%, 92%, 93%, 94%, 95%, 96%, 97%, 98%, 99%, or 100% identical to the amino acid sequence of SEQ ID NO: 245; a first heavy chain comprising an amino acid at least 90%, 91%, 92%, 93%, 94%, 95%, 96%, 97%, 98%, 99%, or 100% identical to the amino acid sequence of SEQ ID NO: 305; a second heavy chain comprising an amino acid at least 90%, 91%, 92%, 93%, 94%, 95%, 96%, 97%, 98%, 99%, or 100% identical to the amino acid sequence of SEQ ID NO: 323; and a second light chain comprising an amino acid at least 90%, 91%, 92%, 93%, 94%, 95%, 96%, 97%, 98%, 99%, or 100% identical to the amino acid sequence of SEQ ID NO: 245.

In some embodiments, the anti-CAIX antibody comprises a first light chain comprising an amino acid at least 90%, 91%, 92%, 93%, 94%, 95%, 96%, 97%, 98%, 99%, or 100% identical to the amino acid sequence of SEQ ID NO: 245; a first heavy chain comprising an amino acid at least 90%, 91%, 92%, 93%, 94%, 95%, 96%, 97%, 98%, 99%, or 100% identical to the amino acid sequence of SEQ ID NO: 305; a second heavy chain comprising an amino acid at least 90%, 91%, 92%, 93%, 94%, 95%, 96%, 97%, 98%, 99%, or 100% identical to the amino acid sequence of SEQ ID NO: 324; and a second light chain comprising an amino acid at least 90%, 91%, 92%, 93%, 94%, 95%, 96%, 97%, 98%, 99%, or 100% identical to the amino acid sequence of SEQ ID NO: 245.

In some embodiments, the anti-CAIX antibody comprises a first light chain comprising an amino acid at least 90%, 91%, 92%, 93%, 94%, 95%, 96%, 97%, 98%, 99%, or 100% identical to the amino acid sequence of SEQ ID NO: 245; a first heavy chain comprising an amino acid at least 90%, 91%, 92%, 93%, 94%, 95%, 96%, 97%, 98%, 99%, or 100% identical to the amino acid sequence of SEQ ID NO: 305; a second heavy chain comprising an amino acid at least 90%, 91%, 92%, 93%, 94%, 95%, 96%, 97%, 98%, 99%, or 100% identical to the amino acid sequence of SEQ ID NO: 325; and a second light chain comprising an amino acid at least 90%, 91%, 92%, 93%, 94%, 95%, 96%, 97%, 98%, 99%, or 100% identical to the amino acid sequence of SEQ ID NO: 245.

In some embodiments, the anti-CAIX antibody comprises a first light chain comprising an amino acid at least 90%, 91%, 92%, 93%, 94%, 95%, 96%, 97%, 98%, 99%, or 100% identical to the amino acid sequence of SEQ ID NO: 245; a first heavy chain comprising an amino acid at least 90%, 91%, 92%, 93%, 94%, 95%, 96%, 97%, 98%, 99%, or 100% identical to the amino acid sequence of SEQ ID NO: 305; a second heavy chain comprising an amino acid at least 90%, 91%, 92%, 93%, 94%, 95%, 96%, 97%, 98%, 99%, or 100% identical to the amino acid sequence of SEQ ID NO: 326; and a second light chain comprising an amino acid at least 90%, 91%, 92%, 93%, 94%, 95%, 96%, 97%, 98%, 99%, or 100% identical to the amino acid sequence of SEQ ID NO: 245.

In some embodiments, the anti-CAIX antibody comprises a first light chain comprising an amino acid at least 90%, 91%, 92%, 93%, 94%, 95%, 96%, 97%, 98%, 99%, or 100% identical to the amino acid sequence of SEQ ID NO: 245; a first heavy chain comprising an amino acid at least 90%, 91%, 92%, 93%, 94%, 95%, 96%, 97%, 98%, 99%, or 100% identical to the amino acid sequence of SEQ ID NO: 305; a second heavy chain comprising an amino acid at least 90%, 91%, 92%, 93%, 94%, 95%, 96%, 97%, 98%, 99%, or 100% identical to the amino acid sequence of SEQ ID NO: 327; and a second light chain comprising an amino acid at least 90%, 91%, 92%, 93%, 94%, 95%, 96%, 97%, 98%, 99%, or 100% identical to the amino acid sequence of SEQ ID NO: 245.

In some embodiments, the anti-CAIX antibody comprises a first light chain comprising an amino acid at least 90%, 91%, 92%, 93%, 94%, 95%, 96%, 97%, 98%, 99%, or 100% identical to the amino acid sequence of SEQ ID NO: 245; a first heavy chain comprising an amino acid at least 90%, 91%, 92%, 93%, 94%, 95%, 96%, 97%, 98%, 99%, or 100% identical to the amino acid sequence of SEQ ID NO: 305; a second heavy chain comprising an amino acid at least 90%, 91%, 92%, 93%, 94%, 95%, 96%, 97%, 98%, 99%, or 100% identical to the amino acid sequence of SEQ ID NO: 328; and a second light chain comprising an amino acid at least 90%, 91%, 92%, 93%, 94%, 95%, 96%, 97%, 98%, 99%, or 100% identical to the amino acid sequence of SEQ ID NO: 245.

In some embodiments, the anti-CAIX antibody comprises a first light chain comprising an amino acid at least 90%, 91%, 92%, 93%, 94%, 95%, 96%, 97%, 98%, 99%, or 100% identical to the amino acid sequence of SEQ ID NO: 245; a first heavy chain comprising an amino acid at least 90%, 91%, 92%, 93%, 94%, 95%, 96%, 97%, 98%, 99%, or 100% identical to the amino acid sequence of SEQ ID NO: 305; a second heavy chain comprising an amino acid at least 90%, 91%, 92%, 93%, 94%, 95%, 96%, 97%, 98%, 99%, or 100% identical to the amino acid sequence of SEQ ID NO: 329; and a second light chain comprising an amino acid at least 90%, 91%, 92%, 93%, 94%, 95%, 96%, 97%, 98%, 99%, or 100% identical to the amino acid sequence of SEQ ID NO: 245.

In some embodiments, the anti-CAIX antibody comprises a first light chain comprising an amino acid at least 90%, 91%, 92%, 93%, 94%, 95%, 96%, 97%, 98%, 99%, or 100% identical to the amino acid sequence of SEQ ID NO: 245; a first heavy chain comprising an amino acid at least 90%, 91%, 92%, 93%, 94%, 95%, 96%, 97%, 98%, 99%, or 100% identical to the amino acid sequence of SEQ ID NO: 305; a second heavy chain comprising an amino acid at least 90%, 91%, 92%, 93%, 94%, 95%, 96%, 97%, 98%, 99%, or 100% identical to the amino acid sequence of SEQ ID NO: 330; and a second light chain comprising an amino acid at least 90%, 91%, 92%, 93%, 94%, 95%, 96%, 97%, 98%, 99%, or 100% identical to the amino acid sequence of SEQ ID NO: 245.

In some embodiments, the anti-CAIX antibody comprises a first light chain comprising an amino acid at least 90%, 91%, 92%, 93%, 94%, 95%, 96%, 97%, 98%, 99%, or 100% identical to the amino acid sequence of SEQ ID NO: 245; a first heavy chain comprising an amino acid at least 90%, 91%, 92%, 93%, 94%, 95%, 96%, 97%, 98%, 99%, or 100% identical to the amino acid sequence of SEQ ID NO: 305; a second heavy chain comprising an amino acid at least 90%, 91%, 92%, 93%, 94%, 95%, 96%, 97%, 98%, 99%, or 100% identical to the amino acid sequence of SEQ ID NO: 331; and a second light chain comprising an amino acid at least 90%, 91%, 92%, 93%, 94%, 95%, 96%, 97%, 98%, 99%, or 100% identical to the amino acid sequence of SEQ ID NO: 245.

In some embodiments, the anti-CAIX antibody comprises a first light chain comprising an amino acid at least 90%, 91%, 92%, 93%, 94%, 95%, 96%, 97%, 98%, 99%, or 100% identical to the amino acid sequence of SEQ ID NO: 245; a first heavy chain comprising an amino acid at least 90%, 91%, 92%, 93%, 94%, 95%, 96%, 97%, 98%, 99%, or 100% identical to the amino acid sequence of SEQ ID NO: 305; a second heavy chain comprising an amino acid at least 90%, 91%, 92%, 93%, 94%, 95%, 96%, 97%, 98%, 99%, or 100% identical to the amino acid sequence of SEQ ID NO: 332; and a second light chain comprising an amino acid at least 90%, 91%, 92%, 93%, 94%, 95%, 96%, 97%, 98%, 99%, or 100% identical to the amino acid sequence of SEQ ID NO: 245.

In some embodiments, the anti-CAIX antibody comprises a first light chain comprising an amino acid at least 90%, 91%, 92%, 93%, 94%, 95%, 96%, 97%, 98%, 99%, or 100% identical to the amino acid sequence of SEQ ID NO: 245; a first heavy chain comprising an amino acid at least 90%, 91%, 92%, 93%, 94%, 95%, 96%, 97%, 98%, 99%, or 100% identical to the amino acid sequence of SEQ ID NO: 305; a second heavy chain comprising an amino acid at least 90%, 91%, 92%, 93%, 94%, 95%, 96%, 97%, 98%, 99%, or 100% identical to the amino acid sequence of SEQ ID NO: 333; and a second light chain comprising an amino acid at least 90%, 91%, 92%, 93%, 94%, 95%, 96%, 97%, 98%, 99%, or 100% identical to the amino acid sequence of SEQ ID NO: 245.

In some embodiments, the anti-CAIX antibody comprises a first light chain comprising an amino acid at least 90%, 91%, 92%, 93%, 94%, 95%, 96%, 97%, 98%, 99%, or 100% identical to the amino acid sequence of SEQ ID NO: 245; a first heavy chain comprising an amino acid at least 90%, 91%, 92%, 93%, 94%, 95%, 96%, 97%, 98%, 99%, or 100% identical to the amino acid sequence of SEQ ID NO: 305; a second heavy chain comprising an amino acid at least 90%, 91%, 92%, 93%, 94%, 95%, 96%, 97%, 98%, 99%, or 100% identical to the amino acid sequence of SEQ ID NO: 334; and a second light chain comprising an amino acid at least 90%, 91%, 92%, 93%, 94%, 95%, 96%, 97%, 98%, 99%, or 100% identical to the amino acid sequence of SEQ ID NO: 245.

In some embodiments, the anti-CAIX antibody comprises a first light chain comprising an amino acid at least 90%, 91%, 92%, 93%, 94%, 95%, 96%, 97%, 98%, 99%, or 100% identical to the amino acid sequence of SEQ ID NO: 245; a first heavy chain comprising an amino acid at least 90%, 91%, 92%, 93%, 94%, 95%, 96%, 97%, 98%, 99%, or 100% identical to the amino acid sequence of SEQ ID NO: 305; a second heavy chain comprising an amino acid at least 90%, 91%, 92%, 93%, 94%, 95%, 96%, 97%, 98%, 99%, or 100% identical to the amino acid sequence of SEQ ID NO: 335; and a second light chain comprising an amino acid at least 90%, 91%, 92%, 93%, 94%, 95%, 96%, 97%, 98%, 99%, or 100% identical to the amino acid sequence of SEQ ID NO: 245.

In some embodiments, the anti-CAIX antibody comprises a first light chain comprising an amino acid at least 90%, 91%, 92%, 93%, 94%, 95%, 96%, 97%, 98%, 99%, or 100% identical to the amino acid sequence of SEQ ID NO: 245; a first heavy chain comprising an amino acid at least 90%, 91%, 92%, 93%, 94%, 95%, 96%, 97%, 98%, 99%, or 100% identical to the amino acid sequence of SEQ ID NO: 305; a second heavy chain comprising an amino acid at least 90%, 91%, 92%, 93%, 94%, 95%, 96%, 97%, 98%, 99%, or 100% identical to the amino acid sequence of SEQ ID NO: 336; and a second light chain comprising an amino acid at least 90%, 91%, 92%, 93%, 94%, 95%, 96%, 97%, 98%, 99%, or 100% identical to the amino acid sequence of SEQ ID NO: 245.

In some embodiments, the anti-CAIX antibody comprises a first light chain comprising an amino acid at least 90%, 91%, 92%, 93%, 94%, 95%, 96%, 97%, 98%, 99%, or 100% identical to the amino acid sequence of SEQ ID NO: 245; a first heavy chain comprising an amino acid at least 90%, 91%, 92%, 93%, 94%, 95%, 96%, 97%, 98%, 99%, or 100% identical to the amino acid sequence of SEQ ID NO: 305; a second heavy chain comprising an amino acid at least 90%, 91%, 92%, 93%, 94%, 95%, 96%, 97%, 98%, 99%, or 100% identical to the amino acid sequence of SEQ ID NO: 337; and a second light chain comprising an amino acid at least 90%, 91%, 92%, 93%, 94%, 95%, 96%, 97%, 98%, 99%, or 100% identical to the amino acid sequence of SEQ ID NO: 245.

In some embodiments, the anti-CAIX antibody comprises a first light chain comprising an amino acid at least 90%, 91%, 92%, 93%, 94%, 95%, 96%, 97%, 98%, 99%, or 100% identical to the amino acid sequence of SEQ ID NO: 245; a first heavy chain comprising an amino acid at least 90%, 91%, 92%, 93%, 94%, 95%, 96%, 97%, 98%, 99%, or 100% identical to the amino acid sequence of SEQ ID NO: 305; a second heavy chain comprising an amino acid at least 90%, 91%, 92%, 93%, 94%, 95%, 96%, 97%, 98%, 99%, or 100% identical to the amino acid sequence of SEQ ID NO: 338; and a second light chain comprising an amino acid at least 90%, 91%, 92%, 93%, 94%, 95%, 96%, 97%, 98%, 99%, or 100% identical to the amino acid sequence of SEQ ID NO: 245.

In some embodiments, the anti-CAIX antibody comprises a first light chain comprising an amino acid at least 90%, 91%, 92%, 93%, 94%, 95%, 96%, 97%, 98%, 99%, or 100% identical to the amino acid sequence of SEQ ID NO: 245; a first heavy chain comprising an amino acid at least 90%, 91%, 92%, 93%, 94%, 95%, 96%, 97%, 98%, 99%, or 100% identical to the amino acid sequence of SEQ ID NO: 305; a second heavy chain comprising an amino acid at least 90%, 91%, 92%, 93%, 94%, 95%, 96%, 97%, 98%, 99%, or 100% identical to the amino acid sequence of SEQ ID NO: 339; and a second light chain comprising an amino acid at least 90%, 91%, 92%, 93%, 94%, 95%, 96%, 97%, 98%, 99%, or 100% identical to the amino acid sequence of SEQ ID NO: 245.

In some embodiments, the anti-CAIX antibody comprises a first light chain comprising an amino acid at least 90%, 91%, 92%, 93%, 94%, 95%, 96%, 97%, 98%, 99%, or 100% identical to the amino acid sequence of SEQ ID NO: 245; a first heavy chain comprising an amino acid at least 90%, 91%, 92%, 93%, 94%, 95%, 96%, 97%, 98%, 99%, or 100% identical to the amino acid sequence of SEQ ID NO: 305; a second heavy chain comprising an amino acid at least 90%, 91%, 92%, 93%, 94%, 95%, 96%, 97%, 98%, 99%, or 100% identical to the amino acid sequence of SEQ ID NO: 340; and a second light chain comprising an amino acid at least 90%, 91%, 92%, 93%, 94%, 95%, 96%, 97%, 98%, 99%, or 100% identical to the amino acid sequence of SEQ ID NO: 245.

In some embodiments, the anti-CAIX antibody comprises a first light chain comprising an amino acid at least 90%, 91%, 92%, 93%, 94%, 95%, 96%, 97%, 98%, 99%, or 100% identical to the amino acid sequence of SEQ ID NO: 245; a first heavy chain comprising an amino acid at least 90%, 91%, 92%, 93%, 94%, 95%, 96%, 97%, 98%, 99%, or 100% identical to the amino acid sequence of SEQ ID NO: 305; a second heavy chain comprising an amino acid at least 90%, 91%, 92%, 93%, 94%, 95%, 96%, 97%, 98%, 99%, or 100% identical to the amino acid sequence of SEQ ID NO: 341; and a second light chain comprising an amino acid at least 90%, 91%, 92%, 93%, 94%, 95%, 96%, 97%, 98%, 99%, or 100% identical to the amino acid sequence of SEQ ID NO: 245.

In some embodiments, the anti-CAIX antibody comprises a first light chain comprising an amino acid at least 90%, 91%, 92%, 93%, 94%, 95%, 96%, 97%, 98%, 99%, or 100% identical to the amino acid sequence of SEQ ID NO: 245; a first heavy chain comprising an amino acid at least 90%, 91%, 92%, 93%, 94%, 95%, 96%, 97%, 98%, 99%, or 100% identical to the amino acid sequence of SEQ ID NO: 305; a second heavy chain comprising an amino acid at least 90%, 91%, 92%, 93%, 94%, 95%, 96%, 97%, 98%, 99%, or 100% identical to the amino acid sequence of SEQ ID NO: 342; and a second light chain comprising an amino acid at least 90%, 91%, 92%, 93%, 94%, 95%, 96%, 97%, 98%, 99%, or 100% identical to the amino acid sequence of SEQ ID NO: 245.

In some embodiments, the anti-CAIX antibody comprises a first light chain comprising an amino acid at least 90%, 91%, 92%, 93%, 94%, 95%, 96%, 97%, 98%, 99%, or 100% identical to the amino acid sequence of SEQ ID NO: 245; a first heavy chain comprising an amino acid at least 90%, 91%, 92%, 93%, 94%, 95%, 96%, 97%, 98%, 99%, or 100% identical to the amino acid sequence of SEQ ID NO: 305; a second heavy chain comprising an amino acid at least 90%, 91%, 92%, 93%, 94%, 95%, 96%, 97%, 98%, 99%, or 100% identical to the amino acid sequence of SEQ ID NO: 343; and a second light chain comprising an amino acid at least 90%, 91%, 92%, 93%, 94%, 95%, 96%, 97%, 98%, 99%, or 100% identical to the amino acid sequence of SEQ ID NO: 245.

In some embodiments, the anti-CAIX antibody comprises a first light chain comprising an amino acid at least 90%, 91%, 92%, 93%, 94%, 95%, 96%, 97%, 98%, 99%, or 100% identical to the amino acid sequence of SEQ ID NO: 245; a first heavy chain comprising an amino acid at least 90%, 91%, 92%, 93%, 94%, 95%, 96%, 97%, 98%, 99%, or 100% identical to the amino acid sequence of SEQ ID NO: 305; a second heavy chain comprising an amino acid at least 90%, 91%, 92%, 93%, 94%, 95%, 96%, 97%, 98%, 99%, or 100% identical to the amino acid sequence of SEQ ID NO: 344; and a second light chain comprising an amino acid at least 90%, 91%, 92%, 93%, 94%, 95%, 96%, 97%, 98%, 99%, or 100% identical to the amino acid sequence of SEQ ID NO: 245.

In some embodiments, the anti-CAIX antibody comprises a first light chain comprising an amino acid at least 90%, 91%, 92%, 93%, 94%, 95%, 96%, 97%, 98%, 99%, or 100% identical to the amino acid sequence of SEQ ID NO: 245; a first heavy chain comprising an amino acid at least 90%, 91%, 92%, 93%, 94%, 95%, 96%, 97%, 98%, 99%, or 100% identical to the amino acid sequence of SEQ ID NO: 306; a second heavy chain comprising an amino acid at least 90%, 91%, 92%, 93%, 94%, 95%, 96%, 97%, 98%, 99%, or 100% identical to the amino acid sequence of SEQ ID NO: 345; and a second light chain comprising an amino acid at least 90%, 91%, 92%, 93%, 94%, 95%, 96%, 97%, 98%, 99%, or 100% identical to the amino acid sequence of SEQ ID NO: 245.

In some embodiments, the anti-CAIX antibody comprises a first light chain comprising an amino acid at least 90%, 91%, 92%, 93%, 94%, 95%, 96%, 97%, 98%, 99%, or 100% identical to the amino acid sequence of SEQ ID NO: 245; a first heavy chain comprising an amino acid at least 90%, 91%, 92%, 93%, 94%, 95%, 96%, 97%, 98%, 99%, or 100% identical to the amino acid sequence of SEQ ID NO: 306; a second heavy chain comprising an amino acid at least 90%, 91%, 92%, 93%, 94%, 95%, 96%, 97%, 98%, 99%, or 100% identical to the amino acid sequence of SEQ ID NO: 346; and a second light chain comprising an amino acid at least 90%, 91%, 92%, 93%, 94%, 95%, 96%, 97%, 98%, 99%, or 100% identical to the amino acid sequence of SEQ ID NO: 245.

In some embodiments, the anti-CAIX antibody comprises a first light chain comprising an amino acid at least 90%, 91%, 92%, 93%, 94%, 95%, 96%, 97%, 98%, 99%, or 100% identical to the amino acid sequence of SEQ ID NO: 245; a first heavy chain comprising an amino acid at least 90%, 91%, 92%, 93%, 94%, 95%, 96%, 97%, 98%, 99%, or 100% identical to the amino acid sequence of SEQ ID NO: 306; a second heavy chain comprising an amino acid at least 90%, 91%, 92%, 93%, 94%, 95%, 96%, 97%, 98%, 99%, or 100% identical to the amino acid sequence of SEQ ID NO: 347; and a second light chain comprising an amino acid at least 90%, 91%, 92%, 93%, 94%, 95%, 96%, 97%, 98%, 99%, or 100% identical to the amino acid sequence of SEQ ID NO: 245.

In some embodiments, the anti-CAIX antibody comprises a first light chain comprising an amino acid at least 90%, 91%, 92%, 93%, 94%, 95%, 96%, 97%, 98%, 99%, or 100% identical to the amino acid sequence of SEQ ID NO: 245; a first heavy chain comprising an amino acid at least 90%, 91%, 92%, 93%, 94%, 95%, 96%, 97%, 98%, 99%, or 100% identical to the amino acid sequence of SEQ ID NO: 306; a second heavy chain comprising an amino acid at least 90%, 91%, 92%, 93%, 94%, 95%, 96%, 97%, 98%, 99%, or 100% identical to the amino acid sequence of SEQ ID NO: 348; and a second light chain comprising an amino acid at least 90%, 91%, 92%, 93%, 94%, 95%, 96%, 97%, 98%, 99%, or 100% identical to the amino acid sequence of SEQ ID NO: 245.

In some embodiments, the anti-CAIX antibody comprises a first light chain comprising an amino acid at least 90%, 91%, 92%, 93%, 94%, 95%, 96%, 97%, 98%, 99%, or 100% identical to the amino acid sequence of SEQ ID NO: 245; a first heavy chain comprising an amino acid at least 90%, 91%, 92%, 93%, 94%, 95%, 96%, 97%, 98%, 99%, or 100% identical to the amino acid sequence of SEQ ID NO: 306; a second heavy chain comprising an amino acid at least 90%, 91%, 92%, 93%, 94%, 95%, 96%, 97%, 98%, 99%, or 100% identical to the amino acid sequence of SEQ ID NO: 349; and a second light chain comprising an amino acid at least 90%, 91%, 92%, 93%, 94%, 95%, 96%, 97%, 98%, 99%, or 100% identical to the amino acid sequence of SEQ ID NO: 245.

In some embodiments, the anti-CAIX antibody comprises a first light chain comprising an amino acid at least 90%, 91%, 92%, 93%, 94%, 95%, 96%, 97%, 98%, 99%, or 100% identical to the amino acid sequence of SEQ ID NO: 245; a first heavy chain comprising an amino acid at least 90%, 91%, 92%, 93%, 94%, 95%, 96%, 97%, 98%, 99%, or 100% identical to the amino acid sequence of SEQ ID NO: 306; a second heavy chain comprising an amino acid at least 90%, 91%, 92%, 93%, 94%, 95%, 96%, 97%, 98%, 99%, or 100% identical to the amino acid sequence of SEQ ID NO: 350; and a second light chain comprising an amino acid at least 90%, 91%, 92%, 93%, 94%, 95%, 96%, 97%, 98%, 99%, or 100% identical to the amino acid sequence of SEQ ID NO: 245.

In some embodiments, the anti-CAIX antibody comprises a first light chain comprising an amino acid at least 90%, 91%, 92%, 93%, 94%, 95%, 96%, 97%, 98%, 99%, or 100% identical to the amino acid sequence of SEQ ID NO: 245; a first heavy chain comprising an amino acid at least 90%, 91%, 92%, 93%, 94%, 95%, 96%, 97%, 98%, 99%, or 100% identical to the amino acid sequence of SEQ ID NO: 306; a second heavy chain comprising an amino acid at least 90%, 91%, 92%, 93%, 94%, 95%, 96%, 97%, 98%, 99%, or 100% identical to the amino acid sequence of SEQ ID NO: 351; and a second light chain comprising an amino acid at least 90%, 91%, 92%, 93%, 94%, 95%, 96%, 97%, 98%, 99%, or 100% identical to the amino acid sequence of SEQ ID NO: 245.

In some embodiments, the anti-CAIX antibody comprises a first light chain comprising an amino acid at least 90%, 91%, 92%, 93%, 94%, 95%, 96%, 97%, 98%, 99%, or 100% identical to the amino acid sequence of SEQ ID NO: 245; a first heavy chain comprising an amino acid at least 90%, 91%, 92%, 93%, 94%, 95%, 96%, 97%, 98%, 99%, or 100% identical to the amino acid sequence of SEQ ID NO: 306; a second heavy chain comprising an amino acid at least 90%, 91%, 92%, 93%, 94%, 95%, 96%, 97%, 98%, 99%, or 100% identical to the amino acid sequence of SEQ ID NO: 352; and a second light chain comprising an amino acid at least 90%, 91%, 92%, 93%, 94%, 95%, 96%, 97%, 98%, 99%, or 100% identical to the amino acid sequence of SEQ ID NO: 245.

In some embodiments, the anti-CAIX antibody comprises a first light chain comprising an amino acid at least 90%, 91%, 92%, 93%, 94%, 95%, 96%, 97%, 98%, 99%, or 100% identical to the amino acid sequence of SEQ ID NO: 245; a first heavy chain comprising an amino acid at least 90%, 91%, 92%, 93%, 94%, 95%, 96%, 97%, 98%, 99%, or 100% identical to the amino acid sequence of SEQ ID NO: 306; a second heavy chain comprising an amino acid at least 90%, 91%, 92%, 93%, 94%, 95%, 96%, 97%, 98%, 99%, or 100% identical to the amino acid sequence of SEQ ID NO: 353; and a second light chain comprising an amino acid at least 90%, 91%, 92%, 93%, 94%, 95%, 96%, 97%, 98%, 99%, or 100% identical to the amino acid sequence of SEQ ID NO: 245.

In some embodiments, the anti-CAIX antibody comprises a first light chain comprising an amino acid at least 90%, 91%, 92%, 93%, 94%, 95%, 96%, 97%, 98%, 99%, or 100% identical to the amino acid sequence of SEQ ID NO: 245; a first heavy chain comprising an amino acid at least 90%, 91%, 92%, 93%, 94%, 95%, 96%, 97%, 98%, 99%, or 100% identical to the amino acid sequence of SEQ ID NO: 306; a second heavy chain comprising an amino acid at least 90%, 91%, 92%, 93%, 94%, 95%, 96%, 97%, 98%, 99%, or 100% identical to the amino acid sequence of SEQ ID NO: 354; and a second light chain comprising an amino acid at least 90%, 91%, 92%, 93%, 94%, 95%, 96%, 97%, 98%, 99%, or 100% identical to the amino acid sequence of SEQ ID NO: 245.

In some embodiments, the anti-CAIX antibody comprises a first light chain comprising an amino acid at least 90%, 91%, 92%, 93%, 94%, 95%, 96%, 97%, 98%, 99%, or 100% identical to the amino acid sequence of SEQ ID NO: 245; a first heavy chain comprising an amino acid at least 90%, 91%, 92%, 93%, 94%, 95%, 96%, 97%, 98%, 99%, or 100% identical to the amino acid sequence of SEQ ID NO: 306; a second heavy chain comprising an amino acid at least 90%, 91%, 92%, 93%, 94%, 95%, 96%, 97%, 98%, 99%, or 100% identical to the amino acid sequence of SEQ ID NO: 355; and a second light chain comprising an amino acid at least 90%, 91%, 92%, 93%, 94%, 95%, 96%, 97%, 98%, 99%, or 100% identical to the amino acid sequence of SEQ ID NO: 245.

In some embodiments, the anti-CAIX antibody comprises a first light chain comprising an amino acid at least 90%, 91%, 92%, 93%, 94%, 95%, 96%, 97%, 98%, 99%, or 100% identical to the amino acid sequence of SEQ ID NO: 245; a first heavy chain comprising an amino acid at least 90%, 91%, 92%, 93%, 94%, 95%, 96%, 97%, 98%, 99%, or 100% identical to the amino acid sequence of SEQ ID NO: 306; a second heavy chain comprising an amino acid at least 90%, 91%, 92%, 93%, 94%, 95%, 96%, 97%, 98%, 99%, or 100% identical to the amino acid sequence of SEQ ID NO: 356; and a second light chain comprising an amino acid at least 90%, 91%, 92%, 93%, 94%, 95%, 96%, 97%, 98%, 99%, or 100% identical to the amino acid sequence of SEQ ID NO: 245.

In some embodiments, the anti-CAIX antibody comprises a first light chain comprising an amino acid at least 90%, 91%, 92%, 93%, 94%, 95%, 96%, 97%, 98%, 99%, or 100% identical to the amino acid sequence of SEQ ID NO: 245; a first heavy chain comprising an amino acid at least 90%, 91%, 92%, 93%, 94%, 95%, 96%, 97%, 98%, 99%, or 100% identical to the amino acid sequence of SEQ ID NO: 306; a second heavy chain comprising an amino acid at least 90%, 91%, 92%, 93%, 94%, 95%, 96%, 97%, 98%, 99%, or 100% identical to the amino acid sequence of SEQ ID NO: 357; and a second light chain comprising an amino acid at least 90%, 91%, 92%, 93%, 94%, 95%, 96%, 97%, 98%, 99%, or 100% identical to the amino acid sequence of SEQ ID NO: 245.

In some embodiments, the anti-CAIX antibody comprises a first light chain comprising an amino acid at least 90%, 91%, 92%, 93%, 94%, 95%, 96%, 97%, 98%, 99%, or 100% identical to the amino acid sequence of SEQ ID NO: 245; a first heavy chain comprising an amino acid at least 90%, 91%, 92%, 93%, 94%, 95%, 96%, 97%, 98%, 99%, or 100% identical to the amino acid sequence of SEQ ID NO: 306; a second heavy chain comprising an amino acid at least 90%, 91%, 92%, 93%, 94%, 95%, 96%, 97%, 98%, 99%, or 100% identical to the amino acid sequence of SEQ ID NO: 358; and a second light chain comprising an amino acid at least 90%, 91%, 92%, 93%, 94%, 95%, 96%, 97%, 98%, 99%, or 100% identical to the amino acid sequence of SEQ ID NO: 245.

In some embodiments, the anti-CAIX antibody comprises a first light chain comprising an amino acid at least 90%, 91%, 92%, 93%, 94%, 95%, 96%, 97%, 98%, 99%, or 100% identical to the amino acid sequence of SEQ ID NO: 245; a first heavy chain comprising an amino acid at least 90%, 91%, 92%, 93%, 94%, 95%, 96%, 97%, 98%, 99%, or 100% identical to the amino acid sequence of SEQ ID NO: 306; a second heavy chain comprising an amino acid at least 90%, 91%, 92%, 93%, 94%, 95%, 96%, 97%, 98%, 99%, or 100% identical to the amino acid sequence of SEQ ID NO: 359; and a second light chain comprising an amino acid at least 90%, 91%, 92%, 93%, 94%, 95%, 96%, 97%, 98%, 99%, or 100% identical to the amino acid sequence of SEQ ID NO: 245.

In some embodiments, the anti-CAIX antibody comprises a first light chain comprising an amino acid at least 90%, 91%, 92%, 93%, 94%, 95%, 96%, 97%, 98%, 99%, or 100% identical to the amino acid sequence of SEQ ID NO: 245; a first heavy chain comprising an amino acid at least 90%, 91%, 92%, 93%, 94%, 95%, 96%, 97%, 98%, 99%, or 100% identical to the amino acid sequence of SEQ ID NO: 306; a second heavy chain comprising an amino acid at least 90%, 91%, 92%, 93%, 94%, 95%, 96%, 97%, 98%, 99%, or 100% identical to the amino acid sequence of SEQ ID NO: 360; and a second light chain comprising an amino acid at least 90%, 91%, 92%, 93%, 94%, 95%, 96%, 97%, 98%, 99%, or 100% identical to the amino acid sequence of SEQ ID NO: 245.

In some embodiments, the anti-CAIX antibody comprises a first light chain comprising an amino acid at least 90%, 91%, 92%, 93%, 94%, 95%, 96%, 97%, 98%, 99%, or 100% identical to the amino acid sequence of SEQ ID NO: 245; a first heavy chain comprising an amino acid at least 90%, 91%, 92%, 93%, 94%, 95%, 96%, 97%, 98%, 99%, or 100% identical to the amino acid sequence of SEQ ID NO: 306; a second heavy chain comprising an amino acid at least 90%, 91%, 92%, 93%, 94%, 95%, 96%, 97%, 98%, 99%, or 100% identical to the amino acid sequence of SEQ ID NO: 361; and a second light chain comprising an amino acid at least 90%, 91%, 92%, 93%, 94%, 95%, 96%, 97%, 98%, 99%, or 100% identical to the amino acid sequence of SEQ ID NO: 245.

In some embodiments, the anti-CAIX antibody comprises a first light chain comprising an amino acid at least 90%, 91%, 92%, 93%, 94%, 95%, 96%, 97%, 98%, 99%, or 100% identical to the amino acid sequence of SEQ ID NO: 245; a first heavy chain comprising an amino acid at least 90%, 91%, 92%, 93%, 94%, 95%, 96%, 97%, 98%, 99%, or 100% identical to the amino acid sequence of SEQ ID NO: 306; a second heavy chain comprising an amino acid at least 90%, 91%, 92%, 93%, 94%, 95%, 96%, 97%, 98%, 99%, or 100% identical to the amino acid sequence of SEQ ID NO: 362; and a second light chain comprising an amino acid at least 90%, 91%, 92%, 93%, 94%, 95%, 96%, 97%, 98%, 99%, or 100% identical to the amino acid sequence of SEQ ID NO: 245.

In some embodiments, the anti-CAIX antibody comprises a first light chain comprising an amino acid at least 90%, 91%, 92%, 93%, 94%, 95%, 96%, 97%, 98%, 99%, or 100% identical to the amino acid sequence of SEQ ID NO: 245; a first heavy chain comprising an amino acid at least 90%, 91%, 92%, 93%, 94%, 95%, 96%, 97%, 98%, 99%, or 100% identical to the amino acid sequence of SEQ ID NO: 306; a second heavy chain comprising an amino acid at least 90%, 91%, 92%, 93%, 94%, 95%, 96%, 97%, 98%, 99%, or 100% identical to the amino acid sequence of SEQ ID NO: 363; and a second light chain comprising an amino acid at least 90%, 91%, 92%, 93%, 94%, 95%, 96%, 97%, 98%, 99%, or 100% identical to the amino acid sequence of SEQ ID NO: 245.

In some embodiments, the anti-CAIX antibody comprises a first light chain comprising an amino acid at least 90%, 91%, 92%, 93%, 94%, 95%, 96%, 97%, 98%, 99%, or 100% identical to the amino acid sequence of SEQ ID NO: 245; a first heavy chain comprising an amino acid at least 90%, 91%, 92%, 93%, 94%, 95%, 96%, 97%, 98%, 99%, or 100% identical to the amino acid sequence of SEQ ID NO: 306; a second heavy chain comprising an amino acid at least 90%, 91%, 92%, 93%, 94%, 95%, 96%, 97%, 98%, 99%, or 100% identical to the amino acid sequence of SEQ ID NO: 364; and a second light chain comprising an amino acid at least 90%, 91%, 92%, 93%, 94%, 95%, 96%, 97%, 98%, 99%, or 100% identical to the amino acid sequence of SEQ ID NO: 245.

In some embodiments, the anti-CAIX antibody comprises a first light chain comprising an amino acid at least 90%, 91%, 92%, 93%, 94%, 95%, 96%, 97%, 98%, 99%, or 100% identical to the amino acid sequence of SEQ ID NO: 245; a first heavy chain comprising an amino acid at least 90%, 91%, 92%, 93%, 94%, 95%, 96%, 97%, 98%, 99%, or 100% identical to the amino acid sequence of SEQ ID NO: 306; a second heavy chain comprising an amino acid at least 90%, 91%, 92%, 93%, 94%, 95%, 96%, 97%, 98%, 99%, or 100% identical to the amino acid sequence of SEQ ID NO: 365; and a second light chain comprising an amino acid at least 90%, 91%, 92%, 93%, 94%, 95%, 96%, 97%, 98%, 99%, or 100% identical to the amino acid sequence of SEQ ID NO: 245.

In some embodiments, the anti-CAIX antibody comprises a first light chain comprising an amino acid at least 90%, 91%, 92%, 93%, 94%, 95%, 96%, 97%, 98%, 99%, or 100% identical to the amino acid sequence of SEQ ID NO: 245; a first heavy chain comprising an amino acid at least 90%, 91%, 92%, 93%, 94%, 95%, 96%, 97%, 98%, 99%, or 100% identical to the amino acid sequence of SEQ ID NO: 306; a second heavy chain comprising an amino acid at least 90%, 91%, 92%, 93%, 94%, 95%, 96%, 97%, 98%, 99%, or 100% identical to the amino acid sequence of SEQ ID NO: 366; and a second light chain comprising an amino acid at least 90%, 91%, 92%, 93%, 94%, 95%, 96%, 97%, 98%, 99%, or 100% identical to the amino acid sequence of SEQ ID NO: 245.

In some embodiments, the anti-CAIX antibody comprises a first light chain comprising an amino acid at least 90%, 91%, 92%, 93%, 94%, 95%, 96%, 97%, 98%, 99%, or 100% identical to the amino acid sequence of SEQ ID NO: 245; a first heavy chain comprising an amino acid at least 90%, 91%, 92%, 93%, 94%, 95%, 96%, 97%, 98%, 99%, or 100% identical to the amino acid sequence of SEQ ID NO: 306; a second heavy chain comprising an amino acid at least 90%, 91%, 92%, 93%, 94%, 95%, 96%, 97%, 98%, 99%, or 100% identical to the amino acid sequence of SEQ ID NO: 367; and a second light chain comprising an amino acid at least 90%, 91%, 92%, 93%, 94%, 95%, 96%, 97%, 98%, 99%, or 100% identical to the amino acid sequence of SEQ ID NO: 245.

In some embodiments, the anti-CAIX antibody comprises a first light chain comprising an amino acid at least 90%, 91%, 92%, 93%, 94%, 95%, 96%, 97%, 98%, 99%, or 100% identical to the amino acid sequence of SEQ ID NO: 245; a first heavy chain comprising an amino acid at least 90%, 91%, 92%, 93%, 94%, 95%, 96%, 97%, 98%, 99%, or 100% identical to the amino acid sequence of SEQ ID NO: 306; a second heavy chain comprising an amino acid at least 90%, 91%, 92%, 93%, 94%, 95%, 96%, 97%, 98%, 99%, or 100% identical to the amino acid sequence of SEQ ID NO: 368; and a second light chain comprising an amino acid at least 90%, 91%, 92%, 93%, 94%, 95%, 96%, 97%, 98%, 99%, or 100% identical to the amino acid sequence of SEQ ID NO: 245.

### 5.4 Methods of Making Polypeptides & Proteins

The hIL-12p40 proteins, hIL-12p35 proteins, schIL-12 proteins, anti-hCAIX antibodies (or any (e.g., one or more) polypeptide thereof), and hIL-12 fusion proteins (or any (e.g., one or more) polypeptide thereof) described herein may be produced using standard methods known in the art. For example, each may be produced by recombinant technology in host cells (e.g., insect cells, mammalian cells, bacteria) that have been transfected or transduced with a nucleic acid expression vector (e.g., plasmid, viral vector (e.g., a baculoviral expression vector)) encoding the hIL-12p40 protein, hIL-12p35 protein, the sch-IL-12 protein, the anti-hCAIX antibody (or any (e.g., one or more) polypeptide thereof), or the hIL-12 fusion protein (or any (e.g., one or more)) polypeptide thereof). Such general methods are common knowledge in the art. The expression vector typically contains an expression cassette that includes nucleic acid sequences capable of bringing about expression of the nucleic acid molecule encoding the protein or polypeptide of interest, such as promoter(s), enhancer(s), polyadenylation signals, and the like. The person of ordinary skill in the art is aware that various promoter and enhancer elements can be used to obtain expression of a nucleic acid molecule in a host cell. For example, promoters can be constitutive or regulated, and can be obtained from various sources, e.g., viruses, prokaryotic or eukaryotic sources, or artificially designed. Post transfection or transduction, host cells containing the expression vector encoding the protein or polypeptide of interest are cultured under conditions conducive to expression of the nucleic acid molecule encoding the antigenic peptide or protein. Culture media is available from various vendors, and a suitable medium can be routinely chosen for a host cell to express a protein or polypeptide of interest. Host cells can be adherent or suspension cultures, and a person of ordinary skill in the art can optimize culture methods for specific host cells selected. For example, suspension cells can be cultured in, for example, bioreactors in e.g., a batch process or a fed-batch process. A number of mammalian cell lines are known in the art and include immortalized cell lines available from the American Type Culture Collection (ATCC), such as, but not limited to, Chinese hamster ovary (CHO) cells, CHO-suspension cells (CHO-S), Hela cells, HEK293, baby hamster kidney (BHK) cells, monkey kidney cells (COS), VERO, HepG2, MadinDarby bovine kidney (MDBK) cells, NOS, U2OS, A549, HT1080, CAD, P19, NIH3T3, L929, N2a, MCF-7, Y79, SO-Rb50, DUKX-X11, and J558L. In some embodiments, the fusion protein is produced in CHO or CHO-S cells.

The produced protein or polypeptide may be isolated from the cell cultures, by, for example, column chromatography in either flow-flow through or bind-and-elute modes. Examples include, but are not limited to, ion exchange resins and affinity resins, such as lentil lectin Sepharose, and mixed mode cation exchange-hydrophobic interaction columns (CEX-HIC). The protein or polypeptide may be concentrated, buffer exchanged by ultrafiltration, and the retentate from the ultrafiltration may be filtered through an appropriate filter, e.g., a 0.22 μm filter. See, e.g., Hacker, David (Ed.), Recombinant Protein Expression in Mammalian Cells: Methods and Protocols (Methods in Molecular Biology), Humana Press (2018); and McPherson et al., “Development of a SARS Coronavirus Vaccine from Recombinant Spike Protein Plus Delta Inulin Adjuvant,” Chapter 4, in Sunil Thomas (ed.), Vaccine Design: Methods and Protocols: Volume 1: Vaccines for Human Diseases, Methods in Molecular Biology, Springer, New York, 2016. See also U.S. Pat. No. 5,762,939, the entire contents of each of which is incorporated herein by reference for all purposes.

The hIL-12p40 proteins described herein, hIL-12p35 proteins described herein, sch-IL12 proteins described herein, anti-hCAIX antibodies (or any (e.g., one or more) polypeptide thereof) described herein, and the hIL-12 fusion proteins (or any (e.g., one or more) polypeptide thereof) described herein may be produced synthetically.

The amino acid sequences of the hIL-12p40 proteins described herein, the hIL-12p35 proteins described herein, the sch-IL12 proteins, the anti-hCAIX antibodies (or any (e.g., one or more) polypeptide thereof) descried herein, or the hIL-12 fusion proteins (or any (e.g., one or more) polypeptide thereof) described herein can be determined, i.e., by repetitive cycles of Edman degradation, followed by amino acid analysis by HPLC. Other methods of amino acid sequencing are also known in the art. Once purified, the functionality of the protein can be assessed using standard assays (e.g., immunoassays, e.g., ELISAs, cell-based assays), known in the art and described herein, see, e.g., Examples 4-15 and 21-23. For example, the bifunctionality of an antibody hIL-12 fusion protein described herein can be assessed using utilizing a bifunctional ELISA.

### 5.5 Polynucleotides, Vectors, Carriers, & Host Cells

In one aspect, provided herein are polynucleotides (e.g., DNA, RNA) encoding a hIL-12p40 protein described herein, a hIL-12p35 protein described herein, a sch-IL12 protein described herein, an anti-hCAIX antibody (or any (e.g., one or more) polypeptide thereof) described herein, or a hIL-12 fusion protein (or any (e.g., one or more) polypeptide thereof) described herein. In some embodiments, the polynucleotide is a DNA polynucleotide or an RNA polynucleotide. In some embodiments, the polynucleotide is an mRNA polynucleotide.

In some embodiments, the polynucleotide is codon optimized. Codon optimization, may be used to match codon frequencies in target and host organisms to ensure proper folding; bias guanosine (G) and/or cytosine I content to increase nucleic acid stability; minimize tandem repeat codons or base runs that may impair gene construction or expression; customize transcriptional and translational control regions; insert or remove protein trafficking sequences; remove/add post translation alteration sites in encoded protein (e.g., glycosylation sites); add, remove, or shuffle protein domains; insert or delete restriction sites; modify ribosome binding sites and mRNA degradation sites; adjust translational rates to allow the various domains of the protein to fold properly; or to reduce or eliminate problem secondary structures within the polynucleotide. In some embodiments, the codon optimized nucleic acid sequence shows one or more of the above (compared to a reference nucleic acid sequence). In some embodiments, the codon optimized nucleic acid sequence shows one or more of improved resistance to in vitro degradation, improved stability in vitro, reduced secondary structures, and/or improved translatability in vitro, compared to a reference nucleic acid sequence. Codon optimization methods, tools, algorithms, and services are known in the art, non-limiting examples include services from GeneArt (Life Technologies) and DNA2.0 (Menlo Park Calif.). In some embodiments, the open reading frame (ORF) sequence is optimized using optimization algorithms. In some embodiments, the nucleic acid sequence is modified to optimize the number of G and/or C nucleotides as compared to a reference nucleic acid sequence. An increase in the number of G and C nucleotides may be generated by substitution of codons containing adenosine (T) or thymidine (T) (or uracil (U)) nucleotides by codons containing G or C nucleotides.

In one aspect, provided herein are vectors comprising a polynucleotide described herein (e.g., a polynucleotide encoding a hIL-12p40 protein described herein, a hIL-12p35 protein described herein, a schIL-12 protein described herein), an anti-hCAIX antibody (or any (e.g., one or more) polypeptide thereof) described herein, or a hIL-12 fusion protein (or any (e.g., one or more) polypeptide thereof) described herein). In some embodiments, the vector is a viral vector. In some embodiments, the vector is a non-viral vector (e.g., a plasmid).

In one aspect, provided herein are carriers comprising a hIL-12p40 protein described herein, a hIL-12p35 protein described herein, a sch-IL12 protein described herein, an anti-hCAIX antibody (or any (e.g., one or more) polypeptide thereof) described herein, or a hIL-12 fusion protein (or any (e.g., one or more) polypeptide thereof) described herein, a polynucleotide described herein, or a vector described herein. Carriers include, but are not limited to, lipid-based carriers such as lipid nanoparticles (LNPs), liposomes, lipoplexes, or nanoliposomes. In some embodiments, the carrier is an LNP, e.g., an LNP described herein.

In one aspect, provided herein are host cells comprising a hIL-12p40 protein described herein, a hIL-12p35 protein described herein, a schIL-12 protein described herein, an anti-hCAIX antibody (or any (e.g., one or more) polypeptide thereof) described herein, or a hIL-12 fusion protein (or any (e.g., one or more) polypeptide thereof) described herein, a polynucleotide described herein, a vector described herein, or a carrier described herein.

### 5.6 Pharmaceutical Compositions

In one aspect, provided herein are pharmaceutical compositions comprising a hIL-12p40 protein described herein, a hIL-12p35 protein described herein, a schIL-12 protein described herein, an anti-hCAIX antibody (or any (e.g., one or more) polypeptide thereof) described herein, or a hIL-12 fusion protein (or any (e.g., one or more) polypeptide thereof) described herein, a polynucleotide described herein, a vector described herein, a host cell described herein, or a carrier described herein, and a pharmaceutically acceptable excipient (see, e.g., Remington's Pharmaceutical Sciences (1990) Mack Publishing Co., Easton, PA, the entire contents of which is incorporated herein by reference for all purposes).

In one aspect, also provided herein are methods of making pharmaceutical compositions described herein comprising providing a hIL-12p40 protein described herein, a hIL-12p35 protein described herein, a schIL-12 protein described herein, an anti-hCAIX antibody (or any (e.g., one or more) polypeptide thereof) described herein, or a hIL-12 fusion protein (or any (e.g., one or more) polypeptide thereof) described herein, a polynucleotide described herein, a vector described herein, a host cell described herein, or a carrier described herein, and formulating it into a pharmaceutically acceptable composition by the addition of one or more pharmaceutically acceptable excipient.

Acceptable excipients (e.g., carriers and stabilizers) are preferably nontoxic to recipients at the dosages and concentrations employed, and include buffers such as phosphate, citrate, or other organic acids; antioxidants including ascorbic acid or methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; or m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, or other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG).

A pharmaceutical composition may be formulated for any route of administration to a subject. Non-limiting embodiments include parenteral administration, such as intramuscular, intradermal, subcutaneous, transcutaneous, or mucosal administration, e.g., inhalation, intranasal, oral, and the like. In one embodiment, the pharmaceutical composition is formulated for administration by intramuscular, intradermal, or subcutaneous injection. In one embodiment, the pharmaceutical composition is formulated for administration by intramuscular injection. In one embodiment, the pharmaceutical composition is formulated for administration by intradermal injection. In one embodiment, the pharmaceutical composition is formulated for administration by subcutaneous injection. Injectables can be prepared in conventional forms, either as liquid solutions or suspensions. The injectables can contain one or more excipients. Exemplary excipients include, for example, water, saline, dextrose, glycerol or ethanol. In addition, if desired, the pharmaceutical compositions to be administered can also contain minor amounts of non-toxic auxiliary substances such as wetting or emulsifying agents, pH buffering agents, stabilizers, solubility enhancers, or other such agents, such as for example, sodium acetate, sorbitan monolaurate, triethanolamine oleate or cyclodextrins. In some embodiments, the pharmaceutical composition is formulated in a single dose. In some embodiments, the pharmaceutical compositions if formulated as a multi-dose.

Pharmaceutically acceptable excipients used in the parenteral preparations described herein include for example, aqueous vehicles, nonaqueous vehicles, antimicrobial agents, isotonic agents, buffers, antioxidants, local anesthetics, suspending and dispersing agents, emulsifying agents, sequestering or chelating agents or other pharmaceutically acceptable substances. Examples of aqueous vehicles, which can be incorporated in one or more of the formulations described herein, include sodium chloride injection, Ringer's injection, isotonic dextrose injection, sterile water injection, dextrose or lactated Ringer's injection. Nonaqueous parenteral vehicles, which can be incorporated in one or more of the formulations described herein, include fixed oils of vegetable origin, cottonseed oil, corn oil, sesame oil or peanut oil. Antimicrobial agents in bacteriostatic or fungistatic concentrations can be added to the parenteral preparations described herein and packaged in multiple-dose containers, which include phenols or cresols, mercurials, benzyl alcohol, chlorobutanol, methyl and propyl p-hydroxybenzoic acid esters, thimerosal, benzalkonium chloride or benzethonium chloride. Isotonic agents, which can be incorporated in one or more of the formulations described herein, include sodium chloride or dextrose. Buffers, which can be incorporated in one or more of the formulations described herein, include phosphate or citrate. Antioxidants, which can be incorporated in one or more of the formulations described herein, include sodium bisulfate. Local anesthetics, which can be incorporated in one or more of the formulations described herein, include procaine hydrochloride. Suspending and dispersing agents, which can be incorporated in one or more of the formulations described herein, include sodium carboxymethylcelluose, hydroxypropyl methylcellulose or polyvinylpyrrolidone. Emulsifying agents, which can be incorporated in one or more of the formulations described herein, include Polysorbate 80 (TWEEN® 80). A sequestering or chelating agent of metal ions, which can be incorporated in one or more of the formulations described herein, is EDTA. Pharmaceutical carriers, which can be incorporated in one or more of the formulations described herein, also include ethyl alcohol, polyethylene glycol or propylene glycol for water miscible vehicles; orsodium hydroxide, hydrochloric acid, citric acid or lactic acid for pH adjustment.

The precise dose to be employed in a pharmaceutical composition will also depend on the route of administration, and the seriousness of the condition caused by it, and should be decided according to the judgment of the practitioner and each subject's circumstances. For example, effective doses may also vary depending upon means of administration, target site, physiological state of the subject (including age, body weight, and health), other medications administered, or whether therapy is prophylactic or therapeutic. Therapeutic dosages are preferably titrated to optimize safety and efficacy.

### 5.7 Methods of Use

Provided herein are various methods of utilizing the hIL-12p40 proteins described herein, hIL-12p35 proteins described herein, schIL-12 proteins described herein, anti-hCAIX antibodies (or any (e.g., one or more) polypeptide thereof) described herein, hIL-12 fusion proteins (or any (e.g., one or more) polypeptide thereof) described herein, polynucleotides described herein, vectors described herein, host cells described herein, carriers described herein, and pharmaceutical compositions described herein. In some embodiments, the methods comprise administration of a hIL-12p40 protein described herein, a hIL-12p35 protein described herein, schIL-12 protein described herein, anti-hCAIX antibody (or any (e.g., one or more) polypeptide thereof) described herein, hIL-12 fusion protein (or any (e.g., one or more) polypeptide thereof) described herein, a polynucleotide described herein, a vector described herein, a host cell described herein, a carrier described herein, or a pharmaceutical composition described herein to a subject. Exemplary subjects include mammals, e.g., humans, non-human mammals, e.g., non-human primates. In some embodiments, the subject is a human.

The dosage of a hIL-12p40 protein described herein, a hIL-12p35 protein described herein, schIL-12 protein described herein, an anti-hCAIX antibody (or any (e.g., one or more) polypeptide thereof) described herein, a hIL-12 fusion protein (or any (e.g., one or more)) polypeptide thereof) described herein, a polynucleotide described herein, a vector described herein, a host cell described herein, a carrier described herein, or a pharmaceutical composition described herein to be administered to a subject in accordance with any of the methods described herein can be determined in accordance with standard techniques known to those of ordinary skill in the art, including the route of administration, the age and weight of the subject, and the type (if any) adjuvant is used.

### 5.7.1 Methods of Delivery

In one aspect, provided herein are methods of delivering (i) a hIL-12p40 protein described herein, (ii) a hIL-12p35 protein described herein, (iii) a schIL-12 protein described herein, (iv) an anti-hCAIX antibody (or any (e.g., one or more)) polypeptide thereof) described herein, (v) a hIL-12 fusion protein (or any (e.g., one or more)) polypeptide thereof) described herein; (vi) a polynucleotide described herein; (vii) a vector described herein; (viii) a host cell described herein; (ix) a carrier described herein; or (x) a pharmaceutical composition described herein to a subject, the method comprising administering the hIL-12p40 protein described herein, the hIL-12p35 protein described herein, the anti-hCAIX antibody (or any (e.g., one or more)) polypeptide thereof) described herein, the hIL-12 fusion protein (or any (e.g., one or more)) polypeptide thereof), the polynucleotide, the vector, the host cell, the carrier, or the pharmaceutical composition to the subject, in an amount and for a time sufficient to deliver the hIL-12p40 protein described herein, the hIL-12p35 protein described herein, the schIL-12 protein described herein, the anti-hCAIX antibody (or any (e.g., one or more)) polypeptide thereof) described herein, the hIL-12 fusion protein (or any (e.g., one or more)) polypeptide thereof), the polynucleotide, the vector, the host cell, the carrier, or the pharmaceutical composition to the subject.

### 5.7.2 Methods of Stimulating T-cell of NK Cell Function

In one aspect, provided herein are methods of stimulating T-cell or NK cell effector function in a subject, the method comprising i) a hIL-12p40 protein described herein, (ii) a hIL-12p35 protein described herein, (iii) a schIL-12 protein described herein, (iv) an anti-hCAIX antibody (or any (e.g., one or more)) polypeptide thereof) described herein, (v) a hIL-12 fusion protein (or any (e.g., one or more)) polypeptide thereof) described herein; (vi) a polynucleotide described herein; (vii) a vector described herein; (viii) a host cell described herein; (ix) a carrier described herein; or (x) a pharmaceutical composition described herein to a subject, in an amount and for a time sufficient to stimulate T-cell or NK cell effector function in the subject. In some embodiments, the effector function is secretion of a cytokine (e.g., IFN-γ). The relevant effector function can be assessed by common methods known in the art and described herein (see, e.g., § 6), for example assessment of secretion of IFN-γ can be conducted using an ELISA, see, e.g., § 6.5 and 6.6.

### 5.7.3 Methods of Preventing or Treating Cancer

In one aspect, provided herein are methods of preventing or treating cancer in a subject, the method comprising i) a hIL-12p40 protein described herein, (ii) a hIL-12p35 protein described herein, (iii) a schIL-12 protein described herein, (iv) an anti-hCAIX antibody (or any (e.g., one or more)) polypeptide thereof) described herein, (v) a hIL-12 fusion protein (or any (e.g., one or more)) polypeptide thereof) described herein; (vi) a polynucleotide described herein; (vii) a vector described herein; (viii) a host cell described herein; (ix) a carrier described herein; or (x) a pharmaceutical composition described herein to a subject, in an amount and for a time sufficient to prevent or treat the cancer in the subject.

In one aspect, provided herein are methods of treating a cancer in a subject, the method comprising: receiving test results that determined the presence of soluble CAIX in a sample from a subject, wherein the sample does not contain cancer cells (or does not contain a substantial number of cancer cells); diagnosing the subject as having a cancer comprising cancer cells expressing CAIX; and i) a hIL-12p40 protein described herein, (ii) a hIL-12p35 protein described herein, (iii) a schIL-12 protein described herein, (iv) an anti-hCAIX antibody (or any (e.g., one or more)) polypeptide thereof) described herein, (v) a hIL-12 fusion protein (or any (e.g., one or more)) polypeptide thereof) described herein; (vi) a polynucleotide described herein; (vii) a vector described herein; (viii) a host cell described herein; (ix) a carrier described herein; or (x) a pharmaceutical composition to the subject, in an amount and for a time sufficient to treat the solid cancer in the subject.

In some embodiments, the cancer is metastatic. In some embodiments, the cancer is recurrent. In some embodiments, the cancer is metastatic and recurrent. In some embodiments, the cancer is refractory to the approved standard of care. In some embodiments, the cancer is refractory to at least one approved standard of care. In some embodiments, the cancer is refractory to at least all approved standard of care therapeutics.

In some embodiments, the cancer is a solid cancer. In some embodiments, the cancer is a hematological malignancy. In some embodiments, the disease is cancer. In some embodiments, the cancer is a hematological malignancy. Exemplary hematological malignancies include, but are not limited to, a leukemia (e.g., Acute lymphocytic leukemia (ALL), Acute myelogenous leukemia (AML), Chronic lymphocytic leukemia (CLL), Chronic myelogenous leukemia (CML)), a lymphoma (e.g., Non-Hodgkin lymphoma, Hodgkin Lymphoma), or a myeloma (e.g., multiple myeloma).

In some embodiments, the cancer is a cancer of epithelial origin. In some embodiments, the cancer is a solid cancer. Exemplary solid tumor cancers include, but are not limited to, lung cancer, central nervous system cancer (e.g., brain cancer or spinal cord cancer, e.g., astrocytoma, glioblastoma), breast cancer, colorectal cancer, colon cancer, rectal cancer, esophageal cancer, kidney cancer, liver cancer, ovarian cancer, pancreatic cancer, prostate cancer, gastric cancer, skin cancer, bladder cancer, uterine cancer, brain cancer, endometrial cancer, lip cancer, oral cancer, mesothelioma, sarcoma, thyroid cancer, thymus cancer, renal cancer, anal cancer, head cancer, neck cancer, and head and neck cancer.

In some embodiments, the cancer is renal cell carcinoma, small bowel cancer, colorectal cancer, bladder cancer, gastric adenocarcinoma, non-small cell lung cancer, esophageal cancer, cervical cancer, small cell lung cancer, head and neck cancer, melanoma, glioblastoma, ovarian cancer, sarcoma, endometrial cancer, gastrointestinal stromal tumor, gastroesophageal junction cancer, triple negative breast cancer, or pancreatic adenocarcinoma.

In some embodiments, the cancer is renal cancer (e.g., renal cell carcinoma), bladder cancer, colorectal cancer, small bowel cancer, esophageal/esophagogastric junction (GEJ) cancer, central nervous system cancer (e.g., brain or spinal cord cancer, e.g., glioblastoma), cervical cancer, gastric cancer, lung cancer (e.g., small cell lung cancer), or gastrointestinal cancer.

In some embodiments, the cancer cells expresses hCAIX on the cell surface. In some embodiments, the cancer has been identified as expressing hCAIX on the cell surface. In some embodiments, a sample of cancer cells (e.g., a biopsy) has determined that at least a portion of the cells in the sample express hCAIX on the cell surface. In some embodiments, the method comprises identifying that at least a portion of cancer cells in a sample of the cancer tissue (e.g., a biopsy) express hCAIX on the cell surface.

In some embodiments, the methods disclosed herein are used in place of standard of care therapies. In certain embodiments, a standard of care therapy is used in combination with any method disclosed herein. Standard-of-care therapies for different types of cancer are well known by persons of skill in the art. For example, the National Comprehensive Cancer Network (NCCN), an alliance of 21 major cancer centers in the USA, publishes the NCCN Clinical Practice Guidelines in Oncology (NCCN GUIDELINES®) that provide detailed up-to-date information on the standard-of-care treatments for a wide variety of cancers. In some embodiments, the methods disclosed herein are used after standard of care therapy has failed.

In some embodiments, the fusion protein is administered to the subject in combination (e.g., before, simultaneously, or after) with one or more prophylactic or therapeutic agents. In some embodiments, the therapeutic agent is an anti-cancer agent. In some embodiments, the therapeutic agent is a chemotherapeutic agent. These therapeutic agents may be in the forms of compounds, antibodies, polypeptides, or polynucleotides.

### 5.7.4 Diagnostic Methods

Further provided herein are diagnostic methods. Some of the methods described herein (e.g., diagnostic methods (see, e.g., § 5.7.4) and therapeutic methods (see, e.g., § 5.7.3)) utilize a sample from a subject. A suitable sample source, size, etc. can be determined by a person of ordinary skill in the art in accordance with use in the selected method. Exemplary subject samples include, but are not limited to, blood, plasma, cell, tissue, saliva sample, and nasal swab. Other samples include, but are not limited to, semen, sputum, mucous, sweat, urine, and feces. In some embodiments, the sample if blood, plasma, or serum. In some embodiments, the sample is blood. In some embodiments, the sample is plasma. In some embodiments, the sample is blood. In some embodiments, the sample is plasma processed from a blood sample.

In one aspect, provided herein are methods of determining the expression of CAIX in cells of a solid cancer in a subject, the method comprising: obtaining the sample from a subject, wherein the sample does not contain cancer cells (or does not contain a substantial number of cancer cells), and determining the presence or absence of soluble CAIX (or a fragment or variant thereof) in the sample.

In one aspect, provided herein are methods of diagnosing a subject with a solid cancer comprising cancer cells expressing CAIX, the method comprising: obtaining the sample from a subject, wherein the sample does not contain cancer cells (or does not contain a substantial number of cancer cells), determining the presence or absence of soluble CAIX (or a fragment or variant thereof) in the sample; and diagnosing the subject as having a solid cancer comprising cancer cells expressing CAIX, if soluble CAIX is determined to be present in the sample.

### 5.8 Kits

In a one aspect, provided herein are kits comprising a hIL-12p40 protein described herein; a hIL-12p35 protein described herein; a schIL-12 protein described herein; an anti-hCAIX antibody (or any (e.g., one or more) polypeptide thereof) described herein; a hIL-12 fusion protein (or any (e.g., one or more) polypeptide thereof) described herein; a polynucleotide described herein; a vector described herein; a host cell described herein; a carrier described herein; or a pharmaceutical composition described herein to a subject, a polynucleotide described herein, a vector described herein, a host cell described herein, a carrier described herein, or a pharmaceutical composition described herein. In addition, the kit may comprise a liquid vehicle for solubilizing or diluting, and/or technical instructions. The technical instructions of the kit may contain information about administration and dosage and subject groups.

In some embodiments, the hIL-12p40 protein described herein; the hIL-12p35 protein described herein; the schIL-12 protein escribed herein; the anti-hCAIX antibody (or any (e.g., one or more) polypeptide thereof) described herein; the hIL-12 fusion protein (or any (e.g., one or more) polypeptide thereof) described herein, the polynucleotide described herein, the vector described herein, the host cell described herein, the carrier described herein, or the pharmaceutical composition described herein is provided in a separate part of the kit, wherein the multispecific protein (or polypeptide thereof) described herein, the polynucleotide described herein, the vector described herein, the host cell described herein, the carrier described herein, or the pharmaceutical composition described herein is optionally lyophilized, spray-dried, or spray-freeze dried. The kit may further contain as a part a vehicle (e.g., buffer solution) for solubilizing the dried or lyophilized hIL-12p40 protein described herein; hIL-12p35 protein described herein; schIL-12 protein escribed herein; anti-hCAIX antibody (or any (e.g., one or more) polypeptide thereof) described herein; hIL-12 fusion protein (or any (e.g., one or more) polypeptide thereof) described herein, polynucleotide described herein, vector described herein, host cell described herein, carrier described herein, or pharmaceutical composition described herein.

In some embodiments, the kit comprises a single dose container. In some embodiments, the kit comprises a multi-dose container. In some embodiments, the kit comprises an administration device (e.g., an injector for intradermal injection or a syringe for intramuscular injection). In some embodiments, the kit comprises adjuvant in a separate container.

Any of the kits described herein may be used in any of the methods described herein (see, e.g., § 5.7).

### 6. EXAMPLES

The examples in this Section (i.e., Section 6) are offered by way of illustration, and not by way of limitation.

### 6.1 Example 1. Generation of Humanized Anti-hCAIX Antibodies

Five different humanized light chain variable regions (VLs) and five different humanized heavy chain variable regions (VHs) of the murine/human chimeric antibody girentuximab (see Table 21) were generated according to the methods described below. Several human framework sequences were identified that were used as “acceptor” frameworks for the girentuximab CDR sequences. As a result, the humanized sequences are expected to be non-immunogenic and retain the canonical structure of the CDR-loops of the parental antibody. For optimal retention of CDR-loop conformations the CDRs were identified using IMGT and Kabat.

For heavy and light chain humanization, databases of Human IgG and IgGk sequences were searched for comparison to the murine VH domain and murine VL domain, respectively, using BLAST search algorithms. Candidate human variable domains were selected from the top 200 BLAST results. Based on a combination of framework homology, maintaining key framework residues and canonical loop structure, the closest human germline IGHV3-11 for the heavy chain and IGKVID-13 for the light chain were selected.

The humanized heavy and light chains were screened for MHC Class II binding epitopes, Fv glycosylation motifs, and deamidation motifs. Subsequently all the combinations of light and heavy chains were transiently expressed and tested for various parameters including expression yield, antigen binding kinetics, and stability.

The amino acid sequence of the human/murine chimeric parental antibody girentuximab (and components thereof) is listed in Table 21.

TABLE 21
The Amino Acid Sequence of Parental Antibody Girentuximab

| | Description | SEQ ID NO |
|---|---|---|
| Girentuximab | VH CDR1 | 237 |
| | VH CDR2 | 238 |
| | VH CDR3 | 239 |
| | VL CDR1 | 240 |
| | VL CDR2 | 241 |
| | VL CDR3 | 242 |
| | VH | 246 |
| | VL | 247 |
| | Heavy Chain | 248 |
| | Light Chain | 249 |

The amino acid sequence of the VH, heavy chain, VL, and the light chain of the generated humanized anti-hCAIX antibodies is set forth in Table 2, above.

The nucleotide sequence of the VH, heavy chain, VL, and the light chain of the generated humanized anti-hCAIX antibodies is set forth in Table 3, above.

The humanized anti-hCAIX antibody comprising HC5 (SEQ ID NO: 7) and LC3 (SEQ ID NO: 15) was further subjected to affinity maturation using the rational affinity maturation platform (RAMP) technology. RAMP combines ‘natural’ library design exploring the somatic hypermutation space of the antibody, generating natural mutations specific to each antibody. Molecular docking and in silico selection were employed for improved antibody binding potential and stability. The methods were designed to balance affinity and developability.

Back mutations were incorporated in both the CDR and framework regions of the antibody and integrated in a combinatorial fashion, which enabled the initial library to be narrowed to an enriched ‘micro-library’ of 95 variants, which were expressed in CHO cells, characterized in full IgG format, and ranked based on affinity and developability attributes. The ‘natural’ design approach and intrinsic consideration for developability enabled RAMP to be used not only to improve affinity but also to optimize for additional attributes e.g., stability, aggregation, pI etc., to improve the overall developability profile of the antibody.

The amino acid sequence of the VH and heavy chain regions of the generated affinity matured humanized anti-hCAIX antibodies is listed is described in Table 22.

TABLE 22
The Amino Acid Sequence of Affinity Matured Humanized
Anti-hCAIX VH and VL Regions

| | |
|---|---|
| Description | SEQ ID NO |

VH

| | |
|---|---|
| HC5 Variant 3 | 7 |
| HC5 Variant 27 | 8 |
| HC5 Variant 29 | 9 |
| HC5 Variant 36 | 9 |
| HC5 Variant 74 | 8 |

VL

| | |
|---|---|
| LC3 Variant 3 | 15 |
| LC3 Variant 27 | 15 |
| LC3 Variant 29 | 15 |
| LC3 Variant 36 | 16 |
| LC3 Variant 74 | 17 |

### 6.2 Example 2. Generation of Anti-hCAIX IL-12 Fusion Proteins

Fusion proteins comprising a hCAIX binding domain and hIL-12 (e.g., hIL-12p35 and hIL-12p40 or a schIL-12) were generated as described below.

The fusion proteins having the BCA316 format (i.e., IL-12p35 fused to a first Fc region and IL-12p40 fused to a second Fc region of the anti-CAIX antibody (see, e.g., FIG. 2 and FIG. 4)) were generated according to the following methods. Briefly, three different polypeptides were co-expressed in CHO cells in vitro, as described below. Each of the polypeptides contained an N-terminal signal peptide that is cleaved to form the mature protein chain and ensures proper secretion into the extracellular media. The three polypeptides were as follows:
1. A humanized light chain (LC3).
2. A first humanized heavy chain (HC5) comprising a knob mutation (T366W) in the CH3 region. The C-terminus of the heavy chain is operably connected to the N-terminus of the mature IL-12p40 polypeptide through a peptide linker (GGGGS)₃ (SEQ ID NO: 72). The hIL-12p40 subunit includes a stop codon at the C-terminus to terminate translation.
3. A second humanized heavy chain (HC5) comprising a set of hole mutations in CH3 domain. The C-terminus of the heavy chain is operably connected to the N-terminus of the mature IL-12p35 polypeptide through a peptide linker (GGGGS)₃ (SEQ ID NO: 7).

The polynucleotides encoding each of the three polypeptides was codon optimized for expression in CHO cells in order to maximize gene expression and subsequently cloned into suitable eukaryotic expression vectors. The polynucleotides encoding the humanized heavy chain knob and humanized heavy chain hole polypeptides were cloned into a single gene expression vector; and the polynucleotide encoding the humanized light chain polypeptide was separately cloned into a different gene expression vector. Together, these two vectors were co-transfected into CHO cells to generate the desired anti-CAIX hIL-12 fusion protein (isolated and purified from the culture supernatant using standard methods). In another expression strategy, all three polynucleotides were cloned into a single gene expression vector, which was subsequently transfected into CHO cells to generate the desired anti-CAIX hIL-12 fusion protein (isolated and purified from the culture supernatant using standard methods).

Fusion proteins having the BCA315 format (i.e., schIL-12 (see, e.g., FIG. 1 and FIG. 3)) were generated according to the following methods. Briefly, three different polypeptides were co-expressed in CHO cells in vitro, as described below. The three polypeptides were as follows:
1. A humanized light chain sequence (LC3).
2. A humanized heavy chain sequence (HC5) with a knob mutation (T366W) in the CH3 region. The C-terminus of the heavy chain is operably connected to the N-terminus of mature hIL-12p35 through a peptide linker (GGGGS)₃ (SEQ ID NO: 7). The C-terminus of the mature hIL-12p35 polypeptide is operably connected to the N-terminus of the mature hIL-12p40 polypeptide through either (i) a short peptide linker (GGGGGGS) (SEQ ID NO: 369) or a long peptide linker of (GGGGS)₃ (SEQ ID NO: 7).
3. A humanized heavy chain sequence (HC5) comprising a set of hole mutations in CH3 domain.

The polynucleotides encoding each of the three polypeptides were codon optimized for expression in CHO cells in order to maximize gene expression and subsequently cloned into suitable eukaryotic expression vectors. The polynucleotides encoding the humanized heavy chain knob and humanized heavy chain hole polypeptides were cloned into a single gene expression vector; and the polynucleotide encoding the humanized light chain polypeptide was separately cloned into a different gene expression vector. Together, these two vector were co-transfected into CHO cells to generate the desired anti-CAIX hIL-12 fusion protein (isolated and purified from the culture supernatant using standard methods). In another expression strategy, all three polynucleotides were cloned into a single gene expression vector, which was subsequently transfected into CHO cells to generate the desired anti-CAIX hIL-12 fusion protein (isolated and purified from the culture supernatant using standard methods).

The amino acid sequence of the generated anti-hCAIX hIL-12 fusion proteins generated is provided in Table 23.

TABLE 23
The Amino Acid Sequence of Generated Anti-hCAIX hIL-12
Fusion Proteins & Polypeptides

| | | SEQ |
|---|---|---|
| | | ID |
| Description | | NO |
| BCA316 | Heavy Chain (HC) (Knob) IL-12p40 | 304 |
| | HC (Hole) IL-12p35 | 305 |
| | Light Chain (LC) | 245 |
| BCA307.16 | Heavy Chain (Knob) IL-12p40 | 304 |
| | Heavy Chain (Hole) IL-12p35 (variant A) | 307 |
| | Light Chain | 245 |
| BCA308.16 | Heavy Chain (Knob) IL-12p40 | 304 |
| | Heavy Chain (Hole) IL-12p35 (variant B) | 308 |
| | Light Chain | 245 |
| BCA323.16 | Heavy Chain (Knob) IL-12p40 | 304 |
| | Heavy Chain (Hole) IL-12p35 (variant C) | 309 |
| | Light Chain | 245 |
| BCA324.16 | Heavy Chain (Knob) IL-12p40 | 304 |
| | Heavy Chain (Hole) IL-12p35 (variant D) | 310 |
| | Light Chain | 245 |
| BCA325.16 | Heavy Chain (Knob) IL-12p40 | 304 |
| | Heavy Chain (Hole) IL-12p35 (variant E) | 311 |
| | Light Chain | 245 |
| BCA309.16 | Heavy Chain (Knob) IL-12p40 (variant A) | 312 |
| | Heavy Chain (Hole) IL-12p35 | 305 |
| | Light Chain | 245 |
| BCA310.16 | Heavy Chain (Knob) IL-12p40 (variant B) | 313 |
| | Heavy Chain (Hole) IL-12p35 | 305 |
| | Light Chain | 245 |
| BCA311.16 | Heavy Chain (Knob) IL-12p40 (variant C) | 314 |
| | Heavy Chain (Hole) IL-12p35 | 305 |
| | Light Chain | 245 |
| BCA317 | Heavy Chain (Knob) IL-12p40 (variant D) | 315 |
| | Heavy Chain (Hole) IL-12p35 | 305 |
| | Light Chain | 245 |
| BCA318.16 | Heavy Chain (Knob) IL-12p40 (variant E) | 316 |
| | Heavy Chain (Hole) IL-12p35 | 305 |
| | Light Chain | 245 |
| BCA319.16 | Heavy Chain (Knob) IL-12p40 (variant F) | 317 |
| | Heavy Chain (Hole) IL-12p35 | 305 |
| | Light Chain | 245 |
| BCA320.16 | Heavy Chain (Knob) IL-12p40 (variant G) | 318 |
| | Heavy Chain (Hole) IL-12p35 | 305 |
| | Light Chain | 245 |
| BCA321.16 | Heavy Chain (Knob) IL-12p40 (variant H) | 319 |
| | Heavy Chain (Hole) IL-12p35 | 305 |
| | Light Chain | 245 |
| BCA322.16 | Heavy Chain (Knob) IL-12p40 (variant I) | 320 |
| | Heavy Chain (Hole) IL-12p35 | 305 |
| | Light Chain | 245 |
| BCA326.16 | Heavy Chain (Knob) IL-12p40 (variant J) | 321 |
| | Heavy Chain (Hole) IL-12p35 | 305 |
| | Light Chain | 245 |
| BCA327.16 | Heavy Chain (Knob) IL-12p40 (variant K) | 322 |
| | Heavy Chain (Hole) IL-12p35 | 305 |
| | Light Chain | 245 |
| BCA328.16 | Heavy Chain (Knob) IL-12p40 (variant L) | 323 |
| | Heavy Chain (Hole) IL-12p35 | 305 |
| | Light Chain | 245 |
| BCA329.16 | Heavy Chain (Knob) IL-12p40 (variant M) | 324 |
| | Heavy Chain (Hole) IL-12p35 | 305 |
| | Light Chain | 245 |
| BCA330.16 | Heavy Chain (Knob) IL-12p40 (variant N) | 325 |
| | Heavy Chain (Hole) IL-12p35 | 305 |
| | Light Chain | 245 |
| BCA331.16 | Heavy Chain (Knob) IL-12p40 (variant O) | 326 |
| | Heavy Chain (Hole) IL-12p35 | 305 |
| | Light Chain | 245 |
| BCA332.16 | Heavy Chain (Knob) IL-12p40 (variant P) | 327 |
| | Heavy Chain (Hole) IL-12p35 | 305 |
| | Light Chain | 245 |
| BCA333.16 | Heavy Chain (Knob) IL-12p40 (variant Q) | 328 |
| | Heavy Chain (Hole) IL-12p35 | 305 |
| | Light Chain | 245 |
| BCA334.16 | Heavy Chain (Knob) IL-12p40 (variant R) | 329 |
| | Heavy Chain (Hole) IL-12p35 | 305 |
| | Light Chain | 245 |
| BCA336.16 | Heavy Chain (Knob) hIL-12p40 (Variant U) | 331 |
| | Heavy Chain (Hole) IL-12p35 | 305 |
| | Light Chain | 245 |
| BCA337.16 | Heavy Chain (Knob) hIL-12p40 (Variant V) | 332 |
| | Heavy Chain (Hole) IL-12p35 | 305 |
| | Light Chain | 245 |
| BCA338.16 | Heavy Chain (Knob) hIL-12p40 (Variant W) | 333 |
| | Heavy Chain (Hole) IL-12p35 | 305 |
| | Light Chain | 245 |
| BCA339.16 | Heavy Chain (Knob) hIL-12p40 (Variant X) | 334 |
| | Heavy Chain (Hole) IL-12p35 | 305 |
| | Light Chain | 245 |
| BCA340.16 | Heavy Chain (Knob) hIL-12p40 (Variant Y) | 335 |
| | Heavy Chain (Hole) IL-12p35 | 305 |
| | Light Chain | 245 |
| BCA341.16 | Heavy Chain (Knob) hIL-12p40 (Variant Z) | 336 |
| | Heavy Chain (Hole) IL-12p35 | 305 |
| | Light Chain | 245 |
| BCA342.16 | Heavy Chain (Knob) hIL-12p40 (Variant AA) | 337 |
| | Heavy Chain (Hole) | 305 |
| | IL-12p35 | |
| | Light Chain | 245 |
| BCA343.16 | Heavy Chain (Knob) hIL-12p40 (Variant BB) | 338 |
| | Heavy Chain (Hole) IL-12p35 | 305 |
| | Light Chain | 245 |
| BCA344.16 | Heavy Chain (Knob) hIL-12p40 (Variant CC) | 339 |
| | Heavy Chain (Hole) IL-12p35 | 305 |
| | Light Chain | 245 |
| BCA345.16 | Heavy Chain (Knob) hIL-12p40 (Variant DD) | 340 |
| | Heavy Chain (Hole) IL-12p35 | 305 |
| | Light Chain | 245 |
| BCA346.16 | Heavy Chain (Knob) hIL-12p40 (Variant EE) | 341 |
| | Heavy Chain (Hole) | 305 |
| | IL-12p35 | |
| | Light Chain | 245 |
| BCA347.16 | Heavy Chain (Knob) hIL-12p40 (Variant FF) | 342 |
| | Heavy Chain (Hole) IL-12p35 | 305 |
| | Light Chain | 245 |
| BCA348.16 | Heavy Chain (Knob) hIL-12p40 (Variant GG) | 343 |
| | Heavy Chain (Hole) IL-12p35 | 305 |
| | Light Chain | 245 |
| BCA315 | Heavy Chain (Knob) scIL-12 | 345 |
| | Heavy Chain (Hole) | 306 |
| | Light Chain | 245 |
| BCA307 | Heavy Chain (Knob) scIL-12 (IL-12p35 Variant A) | 346 |
| | Heavy Chain (Hole) | 306 |
| | Light Chain | 245 |
| BCA308 | Heavy Chain (Knob) scIL-12 (IL-12p35 Variant B) | 347 |
| | Heavy Chain (Hole) | 306 |
| | Light Chain | 245 |
| BCA323 | Heavy Chain (Knob) scIL-12 (IL-12p35 Variant C) | 348 |
| | Heavy Chain (Hole) | 306 |
| | Light Chain | 245 |
| BCA324 | Heavy Chain (Knob) scIL-12 (IL-12p35 Variant D) | 349 |
| | Heavy Chain (Hole) | 306 |
| | Light Chain | 245 |
| BCA325 | Heavy Chain (Knob) scIL-12 (IL-12p35 Variant E) | 350 |
| | Heavy Chain (Hole) | 306 |
| | Light Chain | 245 |
| BCA309 | Heavy Chain (Knob) scIL-12 (IL-12p40 Variant A) | 351 |
| | Heavy Chain (Hole) | 306 |
| | Light Chain | 245 |
| BCA310 | Heavy Chain (Knob) scIL-12 (IL-12p40 Variant B) | 352 |
| | Heavy Chain (Hole) | 306 |
| | Light Chain | 245 |
| BCA311 | Heavy Chain (Knob) scIL-12 (IL-12p35 Variant C) | 353 |
| | Heavy Chain (Hole) | 306 |
| | Light Chain | 245 |
| BCA312 | Heavy Chain (Knob) scIL-12 (IL-12p40 Variant S) | 354 |
| | Heavy Chain (Hole) | 306 |
| | Light Chain | 245 |
| BCA318 | Heavy Chain (Knob) scIL-12 (IL-12p40 Variant E) | 355 |
| | Heavy Chain (Hole) | 306 |
| | Light Chain | 245 |
| BCA319 | Heavy Chain (Knob) scIL-12 (IL-12p40 Variant F) | 356 |
| | Heavy Chain (Hole) | 306 |
| | Light Chain | 245 |
| BCA320 | Heavy Chain (Knob) scIL-12 (IL-12p40 Variant G) | 357 |
| | Heavy Chain (Hole) | 306 |
| | Light Chain | 245 |
| BCA321 | Heavy Chain (Knob) scIL-12 (IL-12p40 Variant H) | 358 |
| | Heavy Chain (Hole) | 306 |
| | Light Chain | 245 |
| BCA322 | Heavy Chain (Knob) scIL-12 (IL-12p40 Variant I) | 359 |
| | Heavy Chain (Hole) | 306 |
| | Light Chain | 245 |
| BCA326 | Heavy Chain (Knob) scIL-12 (IL-12p40 Variant J) | 360 |
| | Heavy Chain (Hole) | 306 |
| | Light Chain | 245 |
| BCA327 | Heavy Chain (Knob) scIL-12 (IL-12p40 Variant A) | 361 |
| | Heavy Chain (Hole) | 306 |
| | Light Chain | 245 |
| BCA328 | Heavy Chain (Knob) scIL-12 (IL-12p40 Variant L) | 362 |
| | Heavy Chain (Hole) | 306 |
| | Light Chain | 245 |
| BCA329 | Heavy Chain (Knob) scIL-12 (IL-12p40 variant M) | 363 |
| | Heavy Chain (Hole) | 306 |
| | Light Chain | 245 |
| BCA330 | Heavy Chain (Knob) scIL-12 (IL-12p40 variant N) | 364 |
| | Heavy Chain (Hole) | 306 |
| | Light Chain | 245 |
| BCA331 | Heavy Chain (Knob) scIL-12 (IL-12p40 variant O) | 365 |
| | Heavy Chain (Hole) | 306 |
| | Light Chain | 245 |
| BCA332 | Heavy Chain (Knob) scIL-12 (IL-12p40 variant P) | 366 |
| | Heavy Chain (Hole) | 306 |
| | Light Chain | 245 |
| BCA333.16 | Heavy Chain (Knob) scIL-12 (IL-12p40 variant Q) | 367 |
| | Heavy Chain (Hole) | 306 |
| | Light Chain | 245 |
| BCA334 | Heavy Chain (Knob) scIL-12 (IL-12p40 variant R) | 368 |
| | Heavy Chain (Hole) | 306 |
| | Light Chain | 245 |

### 6.3 Example 3. Generation of Anti-hCAIX hIL-12p40 Variant Fusion Proteins

The following anti-hCAIX hIL-12p40 fusion proteins were generated according to standard methods. Briefly, the different polypeptide sequences of each fusion protein variant were codon optimized to obtain a DNA polynucleotide counterpart that was cloned into a suitable mammalian expression plasmid. The respective plasmids were transiently co-transfected into Expi-CHO-S cells. The transfected cells were grown for 7-10 days with appropriate feeding as per the manufacturer's protocol. The supernatants of the cell cultures were harvested and the fusion proteins purified using Protein A and SEC affinity columns. The purified proteins were eluted in suitable buffer and subsequently diluted to working concentrations. In some experiments a reference construct was also assessed, including recombinant human IL-12 (rhIL-12) (R&D systems 10018-IL), and an untargeted Fc-hIL-12 fusion protein (SEQ ID NO: 384-385).

Table 24 provides a description of the amino acid modifications contained in the hIL-12 polypeptide of the generated fusion proteins.

TABLE 24
The Amino Acid Sequence of hIL-12p40 Variants and Controls

| | hIL-12p40 Amino Acid Substitution | |
|---|---|---|
| | (numbering relative to the | |
| | amino acid sequence of | |
| Description | SEQ ID NO: 32) | SEQ ID NOS |
| BCA369 | W37A | 370-372 |
| BCA370 | F82A | 371, 372, 373 |
| BCA371 | K217A | 371, 372, 374 |
| BCA372 | K219A | 371, 372, 375 |
| BCA373 | W37A, F82A | 371, 372, 376 |
| BCA374 | W37A, K217A | 371, 372, 377 |
| BCA375 | W37A, K219A | 371, 372, 378 |
| BCA376 | F82A, K217A | 371, 372, 379 |
| BCA377 | F82A, K219A | 371, 372,380 |
| BCA378 | K217A, K219A | 371, 372, 381 |
| BCA356 | W37A/F82A/K219A | 371, 372, 382 |
| BCA351 | None | 371, 372, 383 |
| (wild type hIL-12) | | |

Table 25 provides the amino acid sequence of each fusion protein generated. The signal sequence of each polypeptide chain is underlined.

TABLE 25
The Amino Acid Sequence of hIL-12p40 Variants and Controls

| | | SEQ | |
|---|---|---|---|
| | | ID | |
| Description | | NO | Amino Acid Sequence |
| BCA369 | HC Knob | 370 | MMSFVSLLLVGILFHATQAEVRLVESGGGLVKPGGSLRLSCAAS |
| Anti- | hIL-12p40 | | GFTESNYYMSWIRQAPEKRLELVSAINSDGGITYYLDTVKGRET |
| hCAIX | | | ISRDNAKNSLYLQMNSLRAEDTALFYCARHRSGYFSMDYWGQGT |
| hIL-12 | | | SVTVSSASTKGPSVFPLAPSSKSTSGGTAALGCLVKDYFPEPVT |
| | | | VSWNSGALTSGVHTFPAVLQSSGLYSLSSVVTVPSSSLGTQTYI |
| | | | CNVNHKPSNTKVDKKVEPKSCDKTHTCPPCPAPELLGGPSVFLE |
| | | | PPKPKDTLMISRTPEVTCVVVDVSHEDPEVKENWYVDGVEVHNA |
| | | | KTKPREEQYNSTYRVVSVLTVLHQDWLNGKEYKCKVSNKALPAP |
| | | | IEKTISKAKGQPREPQVYTLPPSRDELTKNQVSLWCLVKGFYPS |
| | | | DIAVEWESNGQPENNYKTTPPVLDSDGSFFLYSKLTVDKSRWQQ |
| | | | GNVFSCSVMHEALHNHYTQKSLSLSPGKGGGGSGGGGSGGGGSI |
| | | | WELKKDVYVVELDAYPDAPGEMVVLTCDTPEEDGITWTLDQSSE |
| | | | VLGSGKTLTIQVKEFGDAGQYTCHKGGEVLSHSLLLLHKKEDGI |
| | | | WSTDILKDQKEPKNKTFLRCEAKNYSGRFTCWWLTTISTDLTES |
| | | | VKSSRGSSDPQGVTCGAATLSAERVRGDNKEYEYSVECQEDSAC |
| | | | PAAEESLPIEVMVDAVHKLKYENYTSSFFIRDIIKPDPPKNLQL |
| | | | KPLKNSRQVEVSWEYPDTWSTPHSYESLTFCVQVQGKSKREKKD |
| | | | RVFTDKTSATVICRKNASISVRAQDRYYSSSWSEWASVPCS |
| | HC Hole | 371 | MMSFVSLLLVGILFHATQAEVRLVESGGGLVKPGGSLRLSCAAS |
| | hIL-12p35 | | GFTFSNYYMSWIRQAPEKRLELVSAINSDGGITYYLDTVKGRET |
| | | | ISRDNAKNSLYLQMNSLRAEDTALFYCARHRSGYFSMDYWGQGT |
| | | | SVTVSSASTKGPSVFPLAPSSKSTSGGTAALGCLVKDYFPEPVT |
| | | | VSWNSGALTSGVHTFPAVLQSSGLYSLSSVVTVPSSSLGTQTYI |
| | | | CNVNHKPSNTKVDKKVEPKSCDKTHTCPPCPAPELLGGPSVELE |
| | | | PPKPKDTLMISRTPEVTCVVVDVSHEDPEVKENWYVDGVEVHNA |
| | | | KTKPREEQYNSTYRVVSVLTVLHQDWLNGKEYKCKVSNKALPAP |
| | | | IEKTISKAKGQPREPQVYTLPPSRDELTKNQVSLSCAVKGFYPS |
| | | | DIAVEWESNGQPENNYKTTPPVLDSDGSFFLVSKLTVDKSRWQQ |
| | | | GNVFSCSVMHEALHNHYTQKSLSLSPGKGGGGSGGGGSGGGGSR |
| | | | NLPVATPDPGMFPCLHHSQNLLRAVSNMLQKARQTLEFYPCTSE |
| | | | EIDHEDITKDKTSTVEACLPLELTKNESCLNSRETSFITNGSCL |
| | | | ASRKTSFMMALCLSSIYEDLKMYQVEFKTMNAKLLMDPKRQIFL |
| | | | DQNMLAVIDELMQALNENSETVPQKSSLEEPDFYKTKIKLCILL |
| | | | HAFRIRAVTIDRVMSYLNAS |
| | LC | 372 | MMSFVSLLLVGILFHATQAEIVMTQSPATLSVSPGERATLSCKA |
| | | | SQNVVSAVAWYQQKPGQSPRLLIYSASNRYTGIPARFSGSGSGT |
| | | | EFTLTISSLQSEDFAAYYCQQYRNYPWTFGGGTKVEIKRTVAAP |
| | | | SVFIFPPSDEQLKSGTASVVCLLNNFYPREAKVQWKVDNALQSG |
| | | | NSQESVTEQDSKDSTYSLSSTLTLSKADYEKHKVYACEVTHQGL |
| | | | SSPVTKSENRGEC |
| BCA370 | HC Knob | 373 | MMSFVSLLLVGILFHATQAEVRLVESGGGLVKPGGSLRLSCAAS |
| Anti- | hIL-12p40 | | GFTFSNYYMSWIRQAPEKRLELVSAINSDGGITYYLDTVKGRFT |
| hCAIX | | | ISRDNAKNSLYLQMNSLRAEDTALFYCARHRSGYFSMDYWGQGT |
| hIL-12 | | | SVTVSSASTKGPSVFPLAPSSKSTSGGTAALGCLVKDYFPEPVT |
| | | | VSWNSGALTSGVHTFPAVLQSSGLYSLSSVVTVPSSSLGTQTYI |
| | | | CNVNHKPSNTKVDKKVEPKSCDKTHTCPPCPAPELLGGPSVELE |
| | | | PPKPKDTLMISRTPEVTCVVVDVSHEDPEVKENWYVDGVEVHNA |
| | | | KTKPREEQYNSTYRVVSVLTVLHQDWLNGKEYKCKVSNKALPAP |
| | | | IEKTISKAKGQPREPQVYTLPPSRDELTKNQVSLWCLVKGFYPS |
| | | | DIAVEWESNGQPENNYKTTPPVLDSDGSFFLYSKLTVDKSRWQQ |
| | | | GNVFSCSVMHEALHNHYTQKSLSLSPGKGGGGSGGGGSGGGGSI |
| | | | WELKKDVYVVELDWYPDAPGEMVVLTCDTPEEDGITWTLDQSSE |
| | | | VLGSGKTLTIQVKEAGDAGQYTCHKGGEVLSHSLLLLHKKEDGI |
| | | | WSTDILKDQKEPKNKTFLRCEAKNYSGRFTCWWLTTISTDLTFS |
| | | | VKSSRGSSDPQGVTCGAATLSAERVRGDNKEYEYSVECQEDSAC |
| | | | PAAEESLPIEVMVDAVHKLKYENYTSSFFIRDIIKPDPPKNLQL |
| | | | KPLKNSRQVEVSWEYPDTWSTPHSYFSLTFCVQVQGKSKREKKD |
| | | | RVFTDKTSATVICRKNASISVRAQDRYYSSSWSEWASVPCS |
| | HC Hole | 371 | MMSFVSLLLVGILFHATQAEVRLVESGGGLVKPGGSLRLSCAAS |
| | hIL-12p35 | | GFTFSNYYMSWIRQAPEKRLELVSAINSDGGITYYLDTVKGRFT |
| | | | ISRDNAKNSLYLQMNSLRAEDTALFYCARHRSGYFSMDYWGQGT |
| | | | SVTVSSASTKGPSVFPLAPSSKSTSGGTAALGCLVKDYFPEPVT |
| | | | VSWNSGALTSGVHTFPAVLQSSGLYSLSSVVTVPSSSLGTQTYI |
| | | | CNVNHKPSNTKVDKKVEPKSCDKTHTCPPCPAPELLGGPSVELF |
| | | | PPKPKDTLMISRTPEVTCVVVDVSHEDPEVKENWYVDGVEVHNA |
| | | | KTKPREEQYNSTYRVVSVLTVLHQDWLNGKEYKCKVSNKALPAP |
| | | | IEKTISKAKGQPREPQVYTLPPSRDELTKNQVSLSCAVKGFYPS |
| | | | DIAVEWESNGQPENNYKTTPPVLDSDGSFFLVSKLTVDKSRWQQ |
| | | | GNVFSCSVMHEALHNHYTQKSLSLSPGKGGGGSGGGGSGGGGSR |
| | | | NLPVATPDPGMFPCLHHSQNLLRAVSNMLQKARQTLEFYPCTSE |
| | | | EIDHEDITKDKTSTVEACLPLELTKNESCLNSRETSFITNGSCL |
| | | | ASRKTSFMMALCLSSIYEDLKMYQVEFKTMNAKLLMDPKRQIFL |
| | | | DQNMLAVIDELMQALNENSETVPQKSSLEEPDFYKTKIKLCILL |
| | | | HAFRIRAVTIDRVMSYLNAS |
| | LC | 372 | MMSFVSLLLVGILFHATQAEIVMTQSPATLSVSPGERATLSCKA |
| | | | SQNVVSAVAWYQQKPGQSPRLLIYSASNRYTGIPARFSGSGSGT |
| | | | EFTLTISSLQSEDFAAYYCQQYRNYPWTFGGGTKVEIKRTVAAP |
| | | | SVFIFPPSDEQLKSGTASVVCLLNNFYPREAKVQWKVDNALQSG |
| | | | NSQESVTEQDSKDSTYSLSSTLTLSKADYEKHKVYACEVTHQGL |
| | | | SSPVTKSENRGEC |
| BCA371 | HC Knob | 374 | MMSFVSLLLVGILFHATQAEVRLVESGGGLVKPGGSLRLSCAAS |
| Anti- | hIL-12p40 | | GFTFSNYYMSWIRQAPEKRLELVSAINSDGGITYYLDTVKGRFT |
| hCAIX | | | ISRDNAKNSLYLQMNSLRAEDTALFYCARHRSGYFSMDYWGQGT |
| hIL-12 | | | SVTVSSASTKGPSVFPLAPSSKSTSGGTAALGCLVKDYFPEPVT |
| | | | VSWNSGALTSGVHTFPAVLQSSGLYSLSSVVTVPSSSLGTQTYI |
| | | | CNVNHKPSNTKVDKKVEPKSCDKTHTCPPCPAPELLGGPSVELF |
| | | | PPKPKDTLMISRTPEVTCVVVDVSHEDPEVKENWYVDGVEVHNA |
| | | | KTKPREEQYNSTYRVVSVLTVLHQDWLNGKEYKCKVSNKALPAP |
| | | | IEKTISKAKGQPREPQVYTLPPSRDELTKNQVSLWCLVKGFYPS |
| | | | DIAVEWESNGQPENNYKTTPPVLDSDGSFFLYSKLTVDKSRWQQ |
| | | | GNVFSCSVMHEALHNHYTQKSLSLSPGKGGGGSGGGGSGGGGSI |
| | | | WELKKDVYVVELDWYPDAPGEMVVLTCDTPEEDGITWTLDQSSE |
| | | | VLGSGKTLTIQVKEFGDAGQYTCHKGGEVLSHSLLLLHKKEDGI |
| | | | WSTDILKDQKEPKNKTFLRCEAKNYSGRFTCWWLTTISTDLTES |
| | | | VKSSRGSSDPQGVTCGAATLSAERVRGDNKEYEYSVECQEDSAC |
| | | | PAAEESLPIEVMVDAVHALKYENYTSSFFIRDIIKPDPPKNLQL |
| | | | KPLKNSRQVEVSWEYPDTWSTPHSYFSLTFCVQVQGKSKREKKD |
| | | | RVFTDKTSATVICRKNASISVRAQDRYYSSSWSEWASVPCS |
| | HC Hole | 371 | MMSFVSLLLVGILFHATQAEVRLVESGGGLVKPGGSLRLSCAAS |
| | hIL-12p35 | | GFTFSNYYMSWIRQAPEKRLELVSAINSDGGITYYLDTVKGRET |
| | | | ISRDNAKNSLYLQMNSLRAEDTALFYCARHRSGYFSMDYWGQGT |
| | | | SVTVSSASTKGPSVFPLAPSSKSTSGGTAALGCLVKDYFPEPVT |
| | | | VSWNSGALTSGVHTFPAVLQSSGLYSLSSVVTVPSSSLGTQTYI |
| | | | CNVNHKPSNTKVDKKVEPKSCDKTHTCPPCPAPELLGGPSVELF |
| | | | PPKPKDTLMISRTPEVTCVVVDVSHEDPEVKENWYVDGVEVHNA |
| | | | KTKPREEQYNSTYRVVSVLTVLHQDWLNGKEYKCKVSNKALPAP |
| | | | IEKTISKAKGQPREPQVYTLPPSRDELTKNQVSLSCAVKGFYPS |
| | | | DIAVEWESNGQPENNYKTTPPVLDSDGSFFLVSKLTVDKSRWQQ |
| | | | GNVFSCSVMHEALHNHYTQKSLSLSPGKGGGGSGGGGSGGGGSR |
| | | | NLPVATPDPGMFPCLHHSQNLLRAVSNMLQKARQTLEFYPCTSE |
| | | | EIDHEDITKDKTSTVEACLPLELTKNESCLNSRETSFITNGSCL |
| | | | ASRKTSFMMALCLSSIYEDLKMYQVEFKTMNAKLLMDPKRQIFL |
| | | | DQNMLAVIDELMQALNENSETVPQKSSLEEPDFYKTKIKLCILL |
| | | | HAFRIRAVTIDRVMSYLNAS |
| | LC | 372 | MMSFVSLLLVGILFHATQAEIVMTQSPATLSVSPGERATLSCKA |
| | | | SQNVVSAVAWYQQKPGQSPRLLIYSASNRYTGIPARFSGSGSGT |
| | | | EFTLTISSLQSEDFAAYYCQQYRNYPWTFGGGTKVEIKRTVAAP |
| | | | SVFIFPPSDEQLKSGTASVVCLLNNFYPREAKVQWKVDNALQSG |
| | | | NSQESVTEQDSKDSTYSLSSTLTLSKADYEKHKVYACEVTHQGL |
| | | | SSPVTKSENRGEC |
| BCA372 | HC Knob | 375 | MMSFVSLLLVGILFHATQAEVRLVESGGGLVKPGGSLRLSCAAS |
| Anti- | hIL-12p40 | | GFTFSNYYMSWIRQAPEKRLELVSAINSDGGITYYLDTVKGRET |
| hCAIX | | | ISRDNAKNSLYLQMNSLRAEDTALFYCARHRSGYFSMDYWGQGT |
| hIL-12 | | | SVTVSSASTKGPSVFPLAPSSKSTSGGTAALGCLVKDYFPEPVT |
| | | | VSWNSGALTSGVHTFPAVLQSSGLYSLSSVVTVPSSSLGTQTYI |
| | | | CNVNHKPSNTKVDKKVEPKSCDKTHTCPPCPAPELLGGPSVELF |
| | | | PPKPKDTLMISRTPEVTCVVVDVSHEDPEVKENWYVDGVEVHNA |
| | | | KTKPREEQYNSTYRVVSVLTVLHQDWLNGKEYKCKVSNKALPAP |
| | | | IEKTISKAKGQPREPQVYTLPPSRDELTKNQVSLWCLVKGFYPS |
| | | | DIAVEWESNGQPENNYKTTPPVLDSDGSFFLYSKLTVDKSRWQQ |
| | | | GNVFSCSVMHEALHNHYTQKSLSLSPGKGGGGSGGGGSGGGGSI |
| | | | WELKKDVYVVELDWYPDAPGEMVVLTCDTPEEDGITWTLDQSSE |
| | | | VLGSGKTLTIQVKEFGDAGQYTCHKGGEVLSHSLLLLHKKEDGI |
| | | | WSTDILKDQKEPKNKTFLRCEAKNYSGRFTCWWLTTISTDLTES |
| | | | VKSSRGSSDPQGVTCGAATLSAERVRGDNKEYEYSVECQEDSAC |
| | | | PAAEESLPIEVMVDAVHKLAYENYTSSFFIRDIIKPDPPKNLQL |
| | | | KPLKNSRQVEVSWEYPDTWSTPHSYFSLTFCVQVQGKSKREKKD |
| | | | RVFTDKTSATVICRKNASISVRAQDRYYSSSWSEWASVPCS |
| | HC Hole | 371 | MMSFVSLLLVGILFHATQAEVRLVESGGGLVKPGGSLRLSCAAS |
| | hIL-12p35 | | GFTFSNYYMSWIRQAPEKRLELVSAINSDGGITYYLDTVKGRET |
| | | | ISRDNAKNSLYLQMNSLRAEDTALFYCARHRSGYFSMDYWGQGT |
| | | | SVTVSSASTKGPSVFPLAPSSKSTSGGTAALGCLVKDYFPEPVT |
| | | | VSWNSGALTSGVHTFPAVLQSSGLYSLSSVVTVPSSSLGTQTYI |
| | | | CNVNHKPSNTKVDKKVEPKSCDKTHTCPPCPAPELLGGPSVELE |
| | | | PPKPKDTLMISRTPEVTCVVVDVSHEDPEVKENWYVDGVEVHNA |
| | | | KTKPREEQYNSTYRVVSVLTVLHQDWLNGKEYKCKVSNKALPAP |
| | | | IEKTISKAKGQPREPQVYTLPPSRDELTKNQVSLSCAVKGFYPS |
| | | | DIAVEWESNGQPENNYKTTPPVLDSDGSFFLVSKLTVDKSRWQQ |
| | | | GNVFSCSVMHEALHNHYTQKSLSLSPGKGGGGSGGGGSGGGGSR |
| | | | NLPVATPDPGMFPCLHHSQNLLRAVSNMLQKARQTLEFYPCTSE |
| | | | EIDHEDITKDKTSTVEACLPLELTKNESCLNSRETSFITNGSCL |
| | | | ASRKTSFMMALCLSSIYEDLKMYQVEFKTMNAKLLMDPKRQIFL |
| | | | DQNMLAVIDELMQALNENSETVPQKSSLEEPDFYKTKIKLCILL |
| | | | HAFRIRAVTIDRVMSYLNAS |
| | LC | 372 | MMSFVSLLLVGILFHATQAEIVMTQSPATLSVSPGERATLSCKA |
| | | | SQNVVSAVAWYQQKPGQSPRLLIYSASNRYTGIPARFSGSGSGT |
| | | | EFTLTISSLQSEDFAAYYCQQYRNYPWTFGGGTKVEIKRTVAAP |
| | | | SVFIFPPSDEQLKSGTASVVCLLNNFYPREAKVQWKVDNALQSG |
| | | | NSQESVTEQDSKDSTYSLSSTLTLSKADYEKHKVYACEVTHQGL |
| | | | SSPVTKSENRGEC |
| BCA373 | HC Knob | 376 | MMSFVSLLLVGILFHATQAEVRLVESGGGLVKPGGSLRLSCAAS |
| Anti- | hIL-12p40 | | GFTFSNYYMSWIRQAPEKRLELVSAINSDGGITYYLDTVKGRET |
| hCAIX | | | ISRDNAKNSLYLQMNSLRAEDTALFYCARHRSGYFSMDYWGQGT |
| hIL-12 | | | SVTVSSASTKGPSVFPLAPSSKSTSGGTAALGCLVKDYFPEPVT |
| | | | VSWNSGALTSGVHTFPAVLQSSGLYSLSSVVTVPSSSLGTQTYI |
| | | | CNVNHKPSNTKVDKKVEPKSCDKTHTCPPCPAPELLGGPSVELE |
| | | | PPKPKDTLMISRTPEVTCVVVDVSHEDPEVKENWYVDGVEVHNA |
| | | | KTKPREEQYNSTYRVVSVLTVLHQDWLNGKEYKCKVSNKALPAP |
| | | | IEKTISKAKGQPREPQVYTLPPSRDELTKNQVSLWCLVKGFYPS |
| | | | DIAVEWESNGQPENNYKTTPPVLDSDGSFFLYSKLTVDKSRWQQ |
| | | | GNVFSCSVMHEALHNHYTQKSLSLSPGKGGGGSGGGGSGGGGSI |
| | | | WELKKDVYVVELDAYPDAPGEMVVLTCDTPEEDGITWTLDQSSE |
| | | | VLGSGKTLTIQVKEAGDAGQYTCHKGGEVLSHSLLLLHKKEDGI |
| | | | WSTDILKDQKEPKNKTFLRCEAKNYSGRFTCWWLTTISTDLTES |
| | | | VKSSRGSSDPQGVTCGAATLSAERVRGDNKEYEYSVECQEDSAC |
| | | | PAAEESLPIEVMVDAVHKLKYENYTSSFFIRDIIKPDPPKNLQL |
| | | | KPLKNSRQVEVSWEYPDTWSTPHSYFSLTFCVQVQGKSKREKKD |
| | | | RVFTDKTSATVICRKNASISVRAQDRYYSSSWSEWASVPCS |
| | HC Hole | 371 | MMSFVSLLLVGILFHATQAEVRLVESGGGLVKPGGSLRLSCAAS |
| | hIL-12p35 | | GFTFSNYYMSWIRQAPEKRLELVSAINSDGGITYYLDTVKGRFT |
| | | | ISRDNAKNSLYLQMNSLRAEDTALFYCARHRSGYFSMDYWGQGT |
| | | | SVTVSSASTKGPSVFPLAPSSKSTSGGTAALGCLVKDYFPEPVT |
| | | | VSWNSGALTSGVHTFPAVLQSSGLYSLSSVVTVPSSSLGTQTYI |
| | | | CNVNHKPSNTKVDKKVEPKSCDKTHTCPPCPAPELLGGPSVELF |
| | | | PPKPKDTLMISRTPEVTCVVVDVSHEDPEVKENWYVDGVEVHNA |
| | | | KTKPREEQYNSTYRVVSVLTVLHQDWLNGKEYKCKVSNKALPAP |
| | | | IEKTISKAKGQPREPQVYTLPPSRDELTKNQVSLSCAVKGFYPS |
| | | | DIAVEWESNGQPENNYKTTPPVLDSDGSFFLVSKLTVDKSRWQQ |
| | | | GNVFSCSVMHEALHNHYTQKSLSLSPGKGGGGSGGGGSGGGGSR |
| | | | NLPVATPDPGMFPCLHHSQNLLRAVSNMLQKARQTLEFYPCTSE |
| | | | EIDHEDITKDKTSTVEACLPLELTKNESCLNSRETSFITNGSCL |
| | | | ASRKTSFMMALCLSSIYEDLKMYQVEFKTMNAKLLMDPKRQIFL |
| | | | DQNMLAVIDELMQALNENSETVPQKSSLEEPDFYKTKIKLCILL |
| | | | HAFRIRAVTIDRVMSYLNAS |
| | LC | 372 | MMSFVSLLLVGILFHATQAEIVMTQSPATLSVSPGERATLSCKA |
| | | | SQNVVSAVAWYQQKPGQSPRLLIYSASNRYTGIPARFSGSGSGT |
| | | | EFTLTISSLQSEDFAAYYCQQYRNYPWTFGGGTKVEIKRTVAAP |
| | | | SVFIFPPSDEQLKSGTASVVCLLNNFYPREAKVQWKVDNALQSG |
| | | | NSQESVTEQDSKDSTYSLSSTLTLSKADYEKHKVYACEVTHQGL |
| | | | SSPVTKSENRGEC |
| BCA374 | HC Knob | 377 | MMSFVSLLLVGILFHATQAEVRLVESGGGLVKPGGSLRLSCAAS |
| Anti- | hIL-12p40 | | GFTFSNYYMSWIRQAPEKRLELVSAINSDGGITYYLDTVKGRET |
| hCAIX | | | ISRDNAKNSLYLQMNSLRAEDTALFYCARHRSGYFSMDYWGQGT |
| hIL-12 | | | SVTVSSASTKGPSVFPLAPSSKSTSGGTAALGCLVKDYFPEPVT |
| | | | VSWNSGALTSGVHTFPAVLQSSGLYSLSSVVTVPSSSLGTQTYI |
| | | | CNVNHKPSNTKVDKKVEPKSCDKTHTCPPCPAPELLGGPSVELE |
| | | | PPKPKDTLMISRTPEVTCVVVDVSHEDPEVKENWYVDGVEVHNA |
| | | | KTKPREEQYNSTYRVVSVLTVLHQDWLNGKEYKCKVSNKALPAP |
| | | | IEKTISKAKGQPREPQVYTLPPSRDELTKNQVSLWCLVKGFYPS |
| | | | DIAVEWESNGQPENNYKTTPPVLDSDGSFFLYSKLTVDKSRWQQ |
| | | | GNVFSCSVMHEALHNHYTQKSLSLSPGKGGGGSGGGGSGGGGSI |
| | | | WELKKDVYVVELDAYPDAPGEMVVLTCDTPEEDGITWTLDQSSE |
| | | | VLGSGKTLTIQVKEFGDAGQYTCHKGGEVLSHSLLLLHKKEDGI |
| | | | WSTDILKDQKEPKNKTFLRCEAKNYSGRFTCWWLTTISTDLTES |
| | | | VKSSRGSSDPQGVTCGAATLSAERVRGDNKEYEYSVECQEDSAC |
| | | | PAAEESLPIEVMVDAVHALKYENYTSSFFIRDIIKPDPPKNLQL |
| | | | KPLKNSRQVEVSWEYPDTWSTPHSYFSLTFCVQVQGKSKREKKD |
| | | | RVFTDKTSATVICRKNASISVRAQDRYYSSSWSEWASVPCS |
| | HC Hole | 371 | MMSFVSLLLVGILFHATQAEVRLVESGGGLVKPGGSLRLSCAAS |
| | hIL-12p35 | | GFTFSNYYMSWIRQAPEKRLELVSAINSDGGITYYLDTVKGRFT |
| | | | ISRDNAKNSLYLQMNSLRAEDTALFYCARHRSGYFSMDYWGQGT |
| | | | SVTVSSASTKGPSVFPLAPSSKSTSGGTAALGCLVKDYFPEPVT |
| | | | VSWNSGALTSGVHTFPAVLQSSGLYSLSSVVTVPSSSLGTQTYI |
| | | | CNVNHKPSNTKVDKKVEPKSCDKTHTCPPCPAPELLGGPSVFLE |
| | | | PPKPKDTLMISRTPEVTCVVVDVSHEDPEVKENWYVDGVEVHNA |
| | | | KTKPREEQYNSTYRVVSVLTVLHQDWLNGKEYKCKVSNKALPAP |
| | | | IEKTISKAKGQPREPQVYTLPPSRDELTKNQVSLSCAVKGFYPS |
| | | | DIAVEWESNGQPENNYKTTPPVLDSDGSFFLVSKLTVDKSRWQQ |
| | | | GNVFSCSVMHEALHNHYTQKSLSLSPGKGGGGSGGGGSGGGGSR |
| | | | NLPVATPDPGMFPCLHHSQNLLRAVSNMLQKARQTLEFYPCTSE |
| | | | EIDHEDITKDKTSTVEACLPLELTKNESCLNSRETSFITNGSCL |
| | | | ASRKTSFMMALCLSSIYEDLKMYQVEFKTMNAKLLMDPKRQIFL |
| | | | DQNMLAVIDELMQALNENSETVPQKSSLEEPDFYKTKIKLCILL |
| | | | HAFRIRAVTIDRVMSYLNAS |
| | LC | 372 | MMSFVSLLLVGILFHATQAEIVMTQSPATLSVSPGERATLSCKA |
| | | | SQNVVSAVAWYQQKPGQSPRLLIYSASNRYTGIPARFSGSGSGT |
| | | | EFTLTISSLQSEDFAAYYCQQYRNYPWTFGGGTKVEIKRTVAAP |
| | | | SVFIFPPSDEQLKSGTASVVCLLNNFYPREAKVQWKVDNALQSG |
| | | | NSQESVTEQDSKDSTYSLSSTLTLSKADYEKHKVYACEVTHQGL |
| | | | SSPVTKSENRGEC |
| BCA375 | HC Knob | 378 | MMSFVSLLLVGILFHATQAEVRLVESGGGLVKPGGSLRLSCAAS |
| Anti- | hIL-12p40 | | GFTFSNYYMSWIRQAPEKRLELVSAINSDGGITYYLDTVKGRFT |
| hCAIX | | | ISRDNAKNSLYLQMNSLRAEDTALFYCARHRSGYFSMDYWGQGT |
| hIL-12 | | | SVTVSSASTKGPSVFPLAPSSKSTSGGTAALGCLVKDYFPEPVT |
| | | | VSWNSGALTSGVHTFPAVLQSSGLYSLSSVVTVPSSSLGTQTYI |
| | | | CNVNHKPSNTKVDKKVEPKSCDKTHTCPPCPAPELLGGPSVFLE |
| | | | PPKPKDTLMISRTPEVTCVVVDVSHEDPEVKENWYVDGVEVHNA |
| | | | KTKPREEQYNSTYRVVSVLTVLHQDWLNGKEYKCKVSNKALPAP |
| | | | IEKTISKAKGQPREPQVYTLPPSRDELTKNQVSLWCLVKGFYPS |
| | | | DIAVEWESNGQPENNYKTTPPVLDSDGSFFLYSKLTVDKSRWQQ |
| | | | GNVFSCSVMHEALHNHYTQKSLSLSPGKGGGGSGGGGGGGGGSI |
| | | | WELKKDVYVVELDAYPDAPGEMVVLTCDTPEEDGITWTLDQSSE |
| | | | VLGSGKTLTIQVKEFGDAGQYTCHKGGEVLSHSLLLLHKKEDGI |
| | | | WSTDILKDQKEPKNKTFLRCEAKNYSGRFTCWWLTTISTDLTES |
| | | | VKSSRGSSDPQGVTCGAATLSAERVRGDNKEYEYSVECQEDSAC |
| | | | PAAEESLPIEVMVDAVHKLAYENYTSSFFIRDIIKPDPPKNLQL |
| | | | KPLKNSRQVEVSWEYPDTWSTPHSYFSLTFCVQVQGKSKREKKD |
| | | | RVFTDKTSATVICRKNASISVRAQDRYYSSSWSEWASVPCS |
| | HC Hole | 371 | MMSFVSLLLVGILFHATQAEVRLVESGGGLVKPGGSLRLSCAAS |
| | hIL-12p35 | | GFTFSNYYMSWIRQAPEKRLELVSAINSDGGITYYLDTVKGRFT |
| | | | ISRDNAKNSLYLQMNSLRAEDTALFYCARHRSGYFSMDYWGQGT |
| | | | SVTVSSASTKGPSVFPLAPSSKSTSGGTAALGCLVKDYFPEPVT |
| | | | VSWNSGALTSGVHTFPAVLQSSGLYSLSSVVTVPSSSLGTQTYI |
| | | | CNVNHKPSNTKVDKKVEPKSCDKTHTCPPCPAPELLGGPSVELE |
| | | | PPKPKDTLMISRTPEVTCVVVDVSHEDPEVKENWYVDGVEVHNA |
| | | | KTKPREEQYNSTYRVVSVLTVLHQDWLNGKEYKCKVSNKALPAP |
| | | | IEKTISKAKGQPREPQVYTLPPSRDELTKNQVSLSCAVKGFYPS |
| | | | DIAVEWESNGQPENNYKTTPPVLDSDGSFFLVSKLTVDKSRWQQ |
| | | | GNVFSCSVMHEALHNHYTQKSLSLSPGKGGGGGGGGSGGGGSR |
| | | | NLPVATPDPGMFPCLHHSQNLLRAVSNMLQKARQTLEFYPCTSE |
| | | | EIDHEDITKDKTSTVEACLPLELTKNESCLNSRETSFITNGSCL |
| | | | ASRKTSFMMALCLSSIYEDLKMYQVEFKTMNAKLLMDPKRQIFL |
| | | | DQNMLAVIDELMQALNENSETVPQKSSLEEPDFYKTKIKLCILL |
| | | | HAFRIRAVTIDRVMSYLNAS |
| | LC | 372 | MMSFVSLLLVGILFHATQAEIVMTQSPATLSVSPGERATLSCKA |
| | | | SQNVVSAVAWYQQKPGQSPRLLIYSASNRYTGIPARFSGSGSGT |
| | | | EFTLTISSLQSEDFAAYYCQQYRNYPWTFGGGTKVEIKRTVAAP |
| | | | SVFIFPPSDEQLKSGTASVVCLLNNFYPREAKVQWKVDNALQSG |
| | | | NSQESVTEQDSKDSTYSLSSTLTLSKADYEKHKVYACEVTHQGL |
| | | | SSPVTKSENRGEC |
| BCA376 | HC Knob | 379 | MMSFVSLLLVGILFHATQAEVRLVESGGGLVKPGGSLRLSCAAS |
| Anti- | hIL-12p40 | | GFTFSNYYMSWIRQAPEKRLELVSAINSDGGITYYLDTVKGRET |
| hCAIX | | | ISRDNAKNSLYLQMNSLRAEDTALFYCARHRSGYFSMDYWGQGT |
| hIL-12 | | | SVTVSSASTKGPSVFPLAPSSKSTSGGTAALGCLVKDYFPEPVT |
| | | | VSWNSGALTSGVHTFPAVLQSSGLYSLSSVVTVPSSSLGTQTYI |
| | | | CNVNHKPSNTKVDKKVEPKSCDKTHTCPPCPAPELLGGPSVELE |
| | | | PPKPKDTLMISRTPEVTCVVVDVSHEDPEVKENWYVDGVEVHNA |
| | | | KTKPREEQYNSTYRVVSVLTVLHQDWLNGKEYKCKVSNKALPAP |
| | | | IEKTISKAKGQPREPQVYTLPPSRDELTKNQVSLWCLVKGFYPS |
| | | | DIAVEWESNGQPENNYKTTPPVLDSDGSFFLYSKLTVDKSRWQQ |
| | | | GNVFSCSVMHEALHNHYTQKSLSLSPGKGGGGSGGGGSGGGGSI |
| | | | WELKKDVYVVELDWYPDAPGEMVVLTCDTPEEDGITWTLDQSSE |
| | | | VLGSGKTLTIQVKEAGDAGQYTCHKGGEVLSHSLLLLHKKEDGI |
| | | | WSTDILKDQKEPKNKTFLRCEAKNYSGRFTCWWLTTISTDLTES |
| | | | VKSSRGSSDPQGVTCGAATLSAERVRGDNKEYEYSVECQEDSAC |
| | | | PAAEESLPIEVMVDAVHALKYENYTSSFFIRDIIKPDPPKNLQL |
| | | | KPLKNSRQVEVSWEYPDTWSTPHSYFSLTFCVQVQGKSKREKKD |
| | | | RVFTDKTSATVICRKNASISVRAQDRYYSSSWSEWASVPCS |
| | HC Hole | 371 | MMSFVSLLLVGILFHATQAEVRLVESGGGLVKPGGSLRLSCAAS |
| | hIL-12p35 | | GFTFSNYYMSWIRQAPEKRLELVSAINSDGGITYYLDTVKGRET |
| | | | ISRDNAKNSLYLQMNSLRAEDTALFYCARHRSGYFSMDYWGQGT |
| | | | SVTVSSASTKGPSVFPLAPSSKSTSGGTAALGCLVKDYFPEPVT |
| | | | VSWNSGALTSGVHTFPAVLQSSGLYSLSSVVTVPSSSLGTQTYI |
| | | | CNVNHKPSNTKVDKKVEPKSCDKTHTCPPCPAPELLGGPSVELE |
| | | | PPKPKDTLMISRTPEVTCVVVDVSHEDPEVKENWYVDGVEVHNA |
| | | | KTKPREEQYNSTYRVVSVLTVLHQDWLNGKEYKCKVSNKALPAP |
| | | | IEKTISKAKGQPREPQVYTLPPSRDELTKNQVSLSCAVKGFYPS |
| | | | DIAVEWESNGQPENNYKTTPPVLDSDGSFFLVSKLTVDKSRWQQ |
| | | | GNVFSCSVMHEALHNHYTQKSLSLSPGKGGGGSGGGGSGGGGSR |
| | | | NLPVATPDPGMFPCLHHSQNLLRAVSNMLQKARQTLEFYPCTSE |
| | | | EIDHEDITKDKTSTVEACLPLELTKNESCLNSRETSFITNGSCL |
| | | | ASRKTSFMMALCLSSIYEDLKMYQVEFKTMNAKLLMDPKRQIFL |
| | | | DQNMLAVIDELMQALNENSETVPQKSSLEEPDFYKTKIKLCILL |
| | | | HAFRIRAVTIDRVMSYLNAS |
| | LC | 372 | MMSFVSLLLVGILFHATQAEIVMTQSPATLSVSPGERATLSCKA |
| | | | SQNVVSAVAWYQQKPGQSPRLLIYSASNRYTGIPARFSGSGSGT |
| | | | EFTLTISSLQSEDFAAYYCQQYRNYPWTFGGGTKVEIKRTVAAP |
| | | | SVFIFPPSDEQLKSGTASVVCLLNNFYPREAKVQWKVDNALQSG |
| | | | NSQESVTEQDSKDSTYSLSSTLTLSKADYEKHKVYACEVTHQGL |
| | | | SSPVTKSENRGEC |
| BCA377 | HC Knob | 380 | MMSFVSLLLVGILFHATQAEVRLVESGGGLVKPGGSLRLSCAAS |
| Anti- | hIL-12p40 | | GFTFSNYYMSWIRQAPEKRLELVSAINSDGGITYYLDTVKGRFT |
| hCAIX | | | ISRDNAKNSLYLQMNSLRAEDTALFYCARHRSGYFSMDYWGQGT |
| hIL-12 | | | SVTVSSASTKGPSVFPLAPSSKSTSGGTAALGCLVKDYFPEPVT |
| | | | VSWNSGALTSGVHTFPAVLQSSGLYSLSSVVTVPSSSLGTQTYI |
| | | | CNVNHKPSNTKVDKKVEPKSCDKTHTCPPCPAPELLGGPSVELF |
| | | | PPKPKDTLMISRTPEVTCVVVDVSHEDPEVKENWYVDGVEVHNA |
| | | | KTKPREEQYNSTYRVVSVLTVLHQDWLNGKEYKCKVSNKALPAP |
| | | | IEKTISKAKGQPREPQVYTLPPSRDELTKNQVSLWCLVKGFYPS |
| | | | DIAVEWESNGQPENNYKTTPPVLDSDGSFFLYSKLTVDKSRWQQ |
| | | | GNVFSCSVMHEALHNHYTQKSLSLSPGKGGGGSGGGGSGGGGSI |
| | | | WELKKDVYVVELDWYPDAPGEMVVLTCDTPEEDGITWTLDQSSE |
| | | | VLGSGKTLTIQVKEAGDAGQYTCHKGGEVLSHSLLLLHKKEDGI |
| | | | WSTDILKDQKEPKNKTFLRCEAKNYSGRFTCWWLTTISTDLTES |
| | | | VKSSRGSSDPQGVTCGAATLSAERVRGDNKEYEYSVECQEDSAC |
| | | | PAAEESLPIEVMVDAVHKLAYENYTSSFFIRDIIKPDPPKNLQL |
| | | | KPLKNSRQVEVSWEYPDTWSTPHSYFSLTFCVQVQGKSKREKKD |
| | | | RVFTDKTSATVICRKNASISVRAQDRYYSSSWSEWASVPCS |
| | HC Hole | 371 | MMSFVSLLLVGILFHATQAEVRLVESGGGLVKPGGSLRLSCAAS |
| | hIL-12p35 | | GFTFSNYYMSWIRQAPEKRLELVSAINSDGGITYYLDTVKGRET |
| | | | ISRDNAKNSLYLQMNSLRAEDTALFYCARHRSGYFSMDYWGQGT |
| | | | SVTVSSASTKGPSVFPLAPSSKSTSGGTAALGCLVKDYFPEPVT |
| | | | VSWNSGALTSGVHTFPAVLQSSGLYSLSSVVTVPSSSLGTQTYI |
| | | | CNVNHKPSNTKVDKKVEPKSCDKTHTCPPCPAPELLGGPSVELF |
| | | | PPKPKDTLMISRTPEVTCVVVDVSHEDPEVKENWYVDGVEVHNA |
| | | | KTKPREEQYNSTYRVVSVLTVLHQDWLNGKEYKCKVSNKALPAP |
| | | | IEKTISKAKGQPREPQVYTLPPSRDELTKNQVSLSCAVKGFYPS |
| | | | DIAVEWESNGQPENNYKTTPPVLDSDGSFFLVSKLTVDKSRWQQ |
| | | | GNVFSCSVMHEALHNHYTQKSLSLSPGKGGGGSGGGGSGGGGSR |
| | | | NLPVATPDPGMFPCLHHSQNLLRAVSNMLQKARQTLEFYPCTSE |
| | | | EIDHEDITKDKTSTVEACLPLELTKNESCLNSRETSFITNGSCL |
| | | | ASRKTSFMMALCLSSIYEDLKMYQVEFKTMNAKLLMDPKRQIFL |
| | | | DQNMLAVIDELMQALNENSETVPQKSSLEEPDFYKTKIKLCILL |
| | | | HAFRIRAVTIDRVMSYLNAS |
| | LC | 372 | MMSFVSLLLVGILFHATQAEIVMTQSPATLSVSPGERATLSCKA |
| | | | SQNVVSAVAWYQQKPGQSPRLLIYSASNRYTGIPARFSGSGSGT |
| | | | EFTLTISSLQSEDFAAYYCQQYRNYPWTFGGGTKVEIKRTVAAP |
| | | | SVFIFPPSDEQLKSGTASVVCLLNNFYPREAKVQWKVDNALQSG |
| | | | NSQESVTEQDSKDSTYSLSSTLTLSKADYEKHKVYACEVTHQGL |
| | | | SSPVTKSENRGEC |
| BCA378 | HC Knob | 381 | MMSFVSLLLVGILFHATQAEVRLVESGGGLVKPGGSLRLSCAAS |
| Anti- | hIL-12p40 | | GFTFSNYYMSWIRQAPEKRLELVSAINSDGGITYYLDTVKGRFT |
| hCAIX | | | ISRDNAKNSLYLQMNSLRAEDTALFYCARHRSGYFSMDYWGQGT |
| hIL-12 | | | SVTVSSASTKGPSVFPLAPSSKSTSGGTAALGCLVKDYFPEPVT |
| | | | VSWNSGALTSGVHTFPAVLQSSGLYSLSSVVTVPSSSLGTQTYI |
| | | | CNVNHKPSNTKVDKKVEPKSCDKTHTCPPCPAPELLGGPSVELE |
| | | | PPKPKDTLMISRTPEVTCVVVDVSHEDPEVKENWYVDGVEVHNA |
| | | | KTKPREEQYNSTYRVVSVLTVLHQDWLNGKEYKCKVSNKALPAP |
| | | | IEKTISKAKGQPREPQVYTLPPSRDELTKNQVSLWCLVKGFYPS |
| | | | DIAVEWESNGQPENNYKTTPPVLDSDGSFFLYSKLTVDKSRWQQ |
| | | | GNVFSCSVMHEALHNHYTQKSLSLSPGKGGGGSGGGGSGGGGSI |
| | | | WELKKDVYVVELDWYPDAPGEMVVLTCDTPEEDGITWTLDQSSE |
| | | | VLGSGKTLTIQVKEFGDAGQYTCHKGGEVLSHSLLLLHKKEDGI |
| | | | WSTDILKDQKEPKNKTFLRCEAKNYSGRFTCWWLTTISTDLTES |
| | | | VKSSRGSSDPQGVTCGAATLSAERVRGDNKEYEYSVECQEDSAC |
| | | | PAAEESLPIEVMVDAVHALAYENYTSSFFIRDIIKPDPPKNLQL |
| | | | KPLKNSRQVEVSWEYPDTWSTPHSYFSLTFCVQVQGKSKREKKD |
| | | | RVFTDKTSATVICRKNASISVRAQDRYYSSSWSEWASVPCS |
| | HC Hole | 371 | MMSFVSLLLVGILFHATQAEVRLVESGGGLVKPGGSLRLSCAAS |
| | hIL-12p35 | | GFTFSNYYMSWIRQAPEKRLELVSAINSDGGITYYLDTVKGRFT |
| | | | ISRDNAKNSLYLQMNSLRAEDTALFYCARHRSGYFSMDYWGQGT |
| | | | SVTVSSASTKGPSVFPLAPSSKSTSGGTAALGCLVKDYFPEPVT |
| | | | VSWNSGALTSGVHTFPAVLQSSGLYSLSSVVTVPSSSLGTQTYI |
| | | | CNVNHKPSNTKVDKKVEPKSCDKTHTCPPCPAPELLGGPSVFLF |
| | | | PPKPKDTLMISRTPEVTCVVVDVSHEDPEVKENWYVDGVEVHNA |
| | | | KTKPREEQYNSTYRVVSVLTVLHQDWLNGKEYKCKVSNKALPAP |
| | | | IEKTISKAKGQPREPQVYTLPPSRDELTKNQVSLSCAVKGFYPS |
| | | | DIAVEWESNGQPENNYKTTPPVLDSDGSFFLVSKLTVDKSRWQQ |
| | | | GNVFSCSVMHEALHNHYTQKSLSLSPGKGGGGSGGGGSGGGGSR |
| | | | NLPVATPDPGMFPCLHHSQNLLRAVSNMLQKARQTLEFYPCTSE |
| | | | EIDHEDITKDKTSTVEACLPLELTKNESCLNSRETSFITNGSCL |
| | | | ASRKTSFMMALCLSSIYEDLKMYQVEFKTMNAKLLMDPKRQIEL |
| | | | DQNMLAVIDELMQALNENSETVPQKSSLEEPDFYKTKIKLCILL |
| | | | HAFRIRAVTIDRVMSYLNAS |
| | LC | 372 | MMSFVSLLLVGILFHATQAEIVMTQSPATLSVSPGERATLSCKA |
| | | | SQNVVSAVAWYQQKPGQSPRLLIYSASNRYTGIPARFSGSGSGT |
| | | | EFTLTISSLQSEDFAAYYCQQYRNYPWTFGGGTKVEIKRTVAAP |
| | | | SVFIFPPSDEQLKSGTASVVCLLNNFYPREAKVQWKVDNALQSG |
| | | | NSQESVTEQDSKDSTYSLSSTLTLSKADYEKHKVYACEVTHQGL |
| | | | SSPVTKSENRGEC |
| BCA356 | HC Knob | 382 | MMSFVSLLLVGILFHATQAEVRLVESGGGLVKPGGSLRLSCAAS |
| Anti- | hIL-12p40 | | GFTFSNYYMSWIRQAPEKRLELVSAINSDGGITYYLDTVKGRET |
| hCAIX | | | ISRDNAKNSLYLQMNSLRAEDTALFYCARHRSGYFSMDYWGQGT |
| hIL-12 | | | SVTVSSASTKGPSVFPLAPSSKSTSGGTAALGCLVKDYFPEPVT |
| | | | VSWNSGALTSGVHTFPAVLQSSGLYSLSSVVTVPSSSLGTQTYI |
| | | | CNVNHKPSNTKVDKKVEPKSCDKTHTCPPCPAPELLGGPSVELE |
| | | | PPKPKDTLMISRTPEVTCVVVDVSHEDPEVKENWYVDGVEVHNA |
| | | | KTKPREEQYNSTYRVVSVLTVLHQDWLNGKEYKCKVSNKALPAP |
| | | | IEKTISKAKGQPREPQVYTLPPSRDELTKNQVSLWCLVKGFYPS |
| | | | DIAVEWESNGQPENNYKTTPPVLDSDGSFFLYSKLTVDKSRWQQ |
| | | | GNVFSCSVMHEALHNHYTQKSLSLSPGKGGGGSGGGGSGGGGSI |
| | | | WELKKDVYVVELDAYPDAPGEMVVLTCDTPEEDGITWTLDQSSE |
| | | | VLGSGKTLTIQVKEAGDAGQYTCHKGGEVLSHSLLLLHKKEDGI |
| | | | WSTDILKDQKEPKNKTFLRCEAKNYSGRFTCWWLTTISTDLTES |
| | | | VKSSRGSSDPQGVTCGAATLSAERVRGDNKEYEYSVECQEDSAC |
| | | | PAAEESLPIEVMVDAVHKLAYENYTSSFFIRDIIKPDPPKNLQL |
| | | | KPLKNSRQVEVSWEYPDTWSTPHSYFSLTFCVQVQGKSKREKKD |
| | | | RVFTDKTSATVICRKNASISVRAQDRYYSSSWSEWASVPCS |
| | HC Hole | 371 | MMSFVSLLLVGILFHATQAEVRLVESGGGLVKPGGSLRLSCAAS |
| | hIL-12p35 | | GFTFSNYYMSWIRQAPEKRLELVSAINSDGGITYYLDTVKGRET |
| | | | ISRDNAKNSLYLQMNSLRAEDTALFYCARHRSGYFSMDYWGQGT |
| | | | SVTVSSASTKGPSVFPLAPSSKSTSGGTAALGCLVKDYFPEPVT |
| | | | VSWNSGALTSGVHTFPAVLQSSGLYSLSSVVTVPSSSLGTQTYI |
| | | | CNVNHKPSNTKVDKKVEPKSCDKTHTCPPCPAPELLGGPSVELF |
| | | | PPKPKDTLMISRTPEVTCVVVDVSHEDPEVKENWYVDGVEVHNA |
| | | | KTKPREEQYNSTYRVVSVLTVLHQDWLNGKEYKCKVSNKALPAP |
| | | | IEKTISKAKGQPREPQVYTLPPSRDELTKNQVSLSCAVKGFYPS |
| | | | DIAVEWESNGQPENNYKTTPPVLDSDGSFFLVSKLTVDKSRWQQ |
| | | | GNVFSCSVMHEALHNHYTQKSLSLSPGKGGGGSGGGGSGGGGSR |
| | | | NLPVATPDPGMFPCLHHSQNLLRAVSNMLQKARQTLEFYPCTSE |
| | | | EIDHEDITKDKTSTVEACLPLELTKNESCLNSRETSFITNGSCL |
| | | | ASRKTSFMMALCLSSIYEDLKMYQVEFKTMNAKLLMDPKRQIFL |
| | | | DQNMLAVIDELMQALNENSETVPQKSSLEEPDFYKTKIKLCILL |
| | | | HAFRIRAVTIDRVMSYLNAS |
| | LC | 372 | MMSFVSLLLVGILFHATQAEIVMTQSPATLSVSPGERATLSCKA |
| | | | SQNVVSAVAWYQQKPGQSPRLLIYSASNRYTGIPARFSGSGSGT |
| | | | EFTLTISSLQSEDFAAYYCQQYRNYPWTFGGGTKVEIKRTVAAP |
| | | | SVFIFPPSDEQLKSGTASVVCLLNNFYPREAKVQWKVDNALQSG |
| | | | NSQESVTEQDSKDSTYSLSSTLTLSKADYEKHKVYACEVTHQGL |
| | | | SSPVTKSENRGEC |
| BCA351 | HC Knob | 383 | MMSFVSLLLVGILFHATQAEVRLVESGGGLVKPGGSLRLSCAAS |
| (wild | hIL-12p40 | | GFTFSNYYMSWIRQAPEKRLELVSAINSDGGITYYLDTVKGRFT |
| type) | | | ISRDNAKNSLYLQMNSLRAEDTALFYCARHRSGYFSMDYWGQGT |
| Anti- | | | SVTVSSASTKGPSVFPLAPSSKSTSGGTAALGCLVKDYFPEPVT |
| hCAIX | | | VSWNSGALTSGVHTFPAVLQSSGLYSLSSVVTVPSSSLGTQTYI |
| hIL-12 | | | CNVNHKPSNTKVDKKVEPKSCDKTHTCPPCPAPELLGGPSVFLE |
| | | | PPKPKDTLMISRTPEVTCVVVDVSHEDPEVKENWYVDGVEVHNA |
| | | | KTKPREEQYNSTYRVVSVLTVLHQDWLNGKEYKCKVSNKALPAP |
| | | | IEKTISKAKGQPREPQVYTLPPSRDELTKNQVSLWCLVKGFYPS |
| | | | DIAVEWESNGQPENNYKTTPPVLDSDGSFFLYSKLTVDKSRWQQ |
| | | | GNVFSCSVMHEALHNHYTQKSLSLSPGKGGGGSGGGGSGGGGSI |
| | | | WELKKDVYVVELDWYPDAPGEMVVLTCDTPEEDGITWTLDQSSE |
| | | | VLGSGKTLTIQVKEFGDAGQYTCHKGGEVLSHSLLLLHKKEDGI |
| | | | WSTDILKDQKEPKNKTFLRCEAKNYSGRFTCWWLTTISTDLTES |
| | | | VKSSRGSSDPQGVTCGAATLSAERVRGDNKEYEYSVECQEDSAC |
| | | | PAAEESLPIEVMVDAVHKLKYENYTSSFFIRDIIKPDPPKNLQL |
| | | | KPLKNSRQVEVSWEYPDTWSTPHSYFSLTFCVQVQGKSKREKKD |
| | | | RVFTDKTSATVICRKNASISVRAQDRYYSSSWSEWASVPCS |
| | HC Hole | 371 | MMSFVSLLLVGILFHATQAEVRLVESGGGLVKPGGSLRLSCAAS |
| | hIL-12p35 | | GFTFSNYYMSWIRQAPEKRLELVSAINSDGGITYYLDTVKGRET |
| | | | ISRDNAKNSLYLQMNSLRAEDTALFYCARHRSGYFSMDYWGQGT |
| | | | SVTVSSASTKGPSVFPLAPSSKSTSGGTAALGCLVKDYFPEPVT |
| | | | VSWNSGALTSGVHTFPAVLQSSGLYSLSSVVTVPSSSLGTQTYI |
| | | | CNVNHKPSNTKVDKKVEPKSCDKTHTCPPCPAPELLGGPSVELF |
| | | | PPKPKDTLMISRTPEVTCVVVDVSHEDPEVKENWYVDGVEVHNA |
| | | | KTKPREEQYNSTYRVVSVLTVLHQDWLNGKEYKCKVSNKALPAP |
| | | | IEKTISKAKGQPREPQVYTLPPSRDELTKNQVSLSCAVKGFYPS |
| | | | DIAVEWESNGQPENNYKTTPPVLDSDGSFFLVSKLTVDKSRWQQ |
| | | | GNVFSCSVMHEALHNHYTQKSLSLSPGKGGGGSGGGGSGGGGSR |
| | | | NLPVATPDPGMFPCLHHSQNLLRAVSNMLQKARQTLEFYPCTSE |
| | | | EIDHEDITKDKTSTVEACLPLELTKNESCLNSRETSFITNGSCL |
| | | | ASRKTSFMMALCLSSIYEDLKMYQVEFKTMNAKLLMDPKRQIFL |
| | | | DQNMLAVIDELMQALNENSETVPQKSSLEEPDFYKTKIKLCILL |
| | | | HAFRIRAVTIDRVMSYLNAS |
| | LC | 372 | MMSFVSLLLVGILFHATQAEIVMTQSPATLSVSPGERATLSCKA |
| | | | SQNVVSAVAWYQQKPGQSPRLLIYSASNRYTGIPARFSGSGSGT |
| | | | EFTLTISSLQSEDFAAYYCQQYRNYPWTFGGGTKVEIKRTVAAP |
| | | | SVFIFPPSDEQLKSGTASVVCLLNNFYPREAKVQWKVDNALQSG |
| | | | NSQESVTEQDSKDSTYSLSSTLTLSKADYEKHKVYACEVTHQGL |
| | | | SSPVTKSENRGEC |
| Fc-IL-12 | Fc- | 384 | MMSFVSLLLVGILFHATQAEPKSCDKTHTCPPCPAPELLGGPSV |
| (WT) | KNOB- | | FLFPPKPKDTLMISRTPEVTCVVVDVSHEDPEVKENWYVDGVEV |
| | Linker | | HNAKTKPREEQYNSTYRVVSVLTVLHQDWLNGKEYKCKVSNKAL |
| | 1-p40: | | PAPIEKTISKAKGQPREPQVYTLPPSRDELTKNQVSLWCLVKGF |
| | | | YPSDIAVEWESNGQPENNYKTTPPVLDSDGSFFLYSKLTVDKSR |
| | | | WQQGNVFSCSVMHEALHNHYTQKSLSLSPGKGGGGSGGGGSGGG |
| | | | GSIWELKKDVYVVELDWYPDAPGEMVVLTCDTPEEDGITWTLDQ |
| | | | SSEVLGSGKTLTIQVKEFGDAGQYTCHKGGEVLSHSLLLLHKKE |
| | | | DGIWSTDILKDQKEPKNKTFLRCEAKNYSGRFTCWWLTTISTDL |
| | | | TFSVKSSRGSSDPQGVTCGAATLSAERVRGDNKEYEYSVECQED |
| | | | SACPAAEESLPIEVMVDAVHKLKYENYTSSFFIRDIIKPDPPKN |
| | | | LQLKPLKNSRQVEVSWEYPDTWSTPHSYFSLTFCVQVQGKSKRE |
| | | | KKDRVFTDKTSATVICRKNASISVRAQDRYYSSSWSEWASVPCS |
| | Fc-HOLE- | 385 | MMSFVSLLLVGILFHATQAEPKSCDKTHTCPPCPAPELLGGPSV |
| | Linker | | FLFPPKPKDTLMISRTPEVTCVVVDVSHEDPEVKENWYVDGVEV |
| | 1-p35: | | HNAKTKPREEQYNSTYRVVSVLTVLHQDWLNGKEYKCKVSNKAL |
| | | | PAPIEKTISKAKGQPREPQVYTLPPSRDELTKNQVSLSCAVKGF |
| | | | YPSDIAVEWESNGQPENNYKTTPPVLDSDGSFFLVSKLTVDKSR |
| | | | WQQGNVFSCSVMHEALHNHYTQKSLSLSPGKGGGGSGGGGSGGG |
| | | | GSRNLPVATPDPGMFPCLHHSQNLLRAVSNMLQKARQTLEFYPC |
| | | | TSEEIDHEDITKDKTSTVEACLPLELTKNESCLNSRETSFITNG |
| | | | SCLASRKTSEMMALCLSSIYEDLKMYQVEFKTMNAKLLMDPKRQ |
| | | | IFLDQNMLAVIDELMQALNENSETVPQKSSLEEPDFYKTKIKLC |
| | | | ILLHAFRIRAVTIDRVMSYLNAS |

The biological activity of each of the fusion proteins comprising a hIL-12p40 variant in Table 25 was evaluated for: (1) activation of STAT4 signalling in HEK-IL-12 reporter cell line (2) IFN-γ release by PHA stimulated hPBMCs; and (3) IFN-γ release by IL-2 primed NK cells. Each assay and the results are described fully in Examples 4-7, respectively.

### 6.4 Example 4. Evaluation of hIL-12p40 Variant Activity-pSTAT4 Signaling

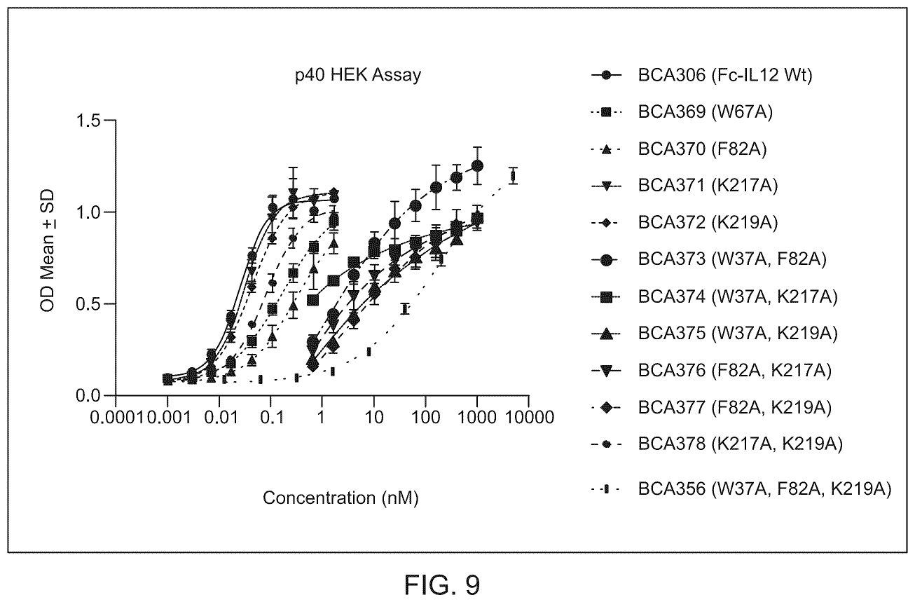

Signaling through the hIL-12 receptor of each of the hIL-12p40 variants set forth in Table 25 was evaluated using a HEK-Blue™ Reporter assay (InvivoGen (#hkb-IL12)). Briefly, HEK-Blue™ IL-12 cells utilized in the assay express a STAT4-inducible SEAP reporter gene. The binding of IL-12 to the IL-12R on the surface of HEK-Blue™ IL-12 cells triggers a signalling cascade leading to the activation (and phosphorylation) of STAT-4 with the subsequent production of the SEAP reporter protein. Detection of SEAP in the supernatant of HEK-Blue™ IL-12 cells can be readily assessed using QUANTI-Blue™ Solution. Here, HEK-Blue cells were incubated with various concentrations of each of the indicated hIL-12p40 variants for 18-24 hours in cell culture plates. The plates were read after addition of Quanti Blue solution. The IL-12 activity of hIL-12p40 variants is presented in FIG. 9 and the fold attenuation of IL-12 activity over wild type Fc-IL-12 is presented in Table 26.

TABLE 26
Fold Attenuation of hIL-12p40 Variants over Fc-IL-12 Control

| | Std | Sample | Fold Attenuation |
|---|---|---|---|
| Description | (EC50) | (EC50) | (Over Fc-IL-12 Control) |
| BCA371 | 0.021 | 0.035 | 2 |
| BCA372 | 0.024 | 0.048 | 2 |
| BCA378 | 0.022 | 0.102 | 5 |
| BCA369 | 0.025 | 0.19 | 8 |
| BCA370 | 0.022 | 0.401 | 18 |
| BCA374 | 0.024 | 1.041 | 43 |
| BCA373 | 0.021 | 4.344 | 207 |
| BCA376 | 0.019 | 4.058 | 214 |
| BCA375 | 0.024 | 7.662 | 319 |
| BCA377 | 0.021 | 9.668 | 460 |
| BCA356 | 0.018 | 131.4 | 7300 |

As shown in FIG. 9 and Table 26, BCA356 showed relatively higher attenuation (7300-fold) compared to either the single or double hIL-12p40 variants.

### 6.5 Example 5. Evaluation of hIL-12p40 Variant Activity-IFN-γ Release by PHA Stimulated PBMCs

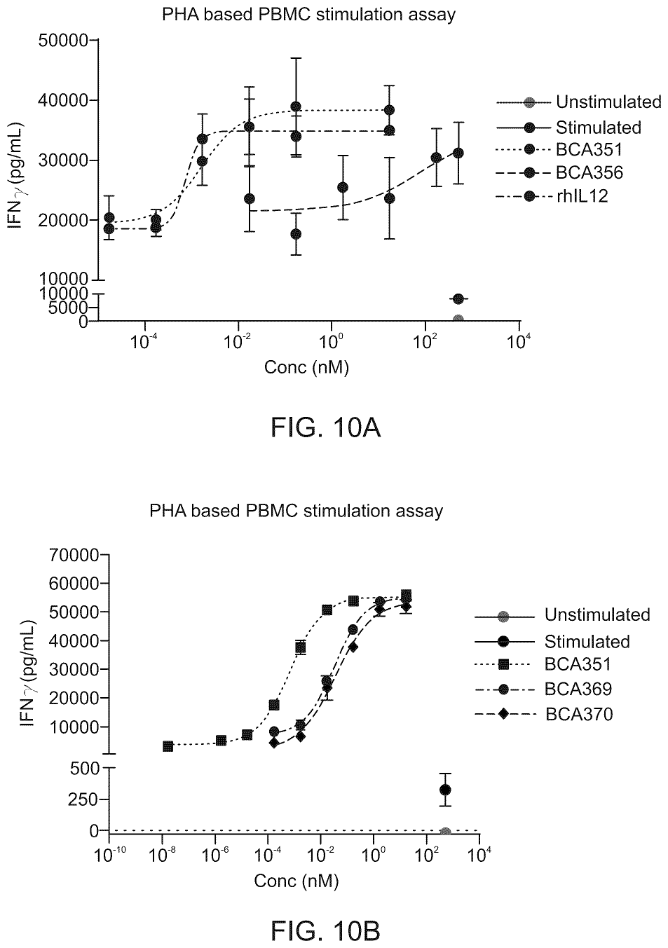

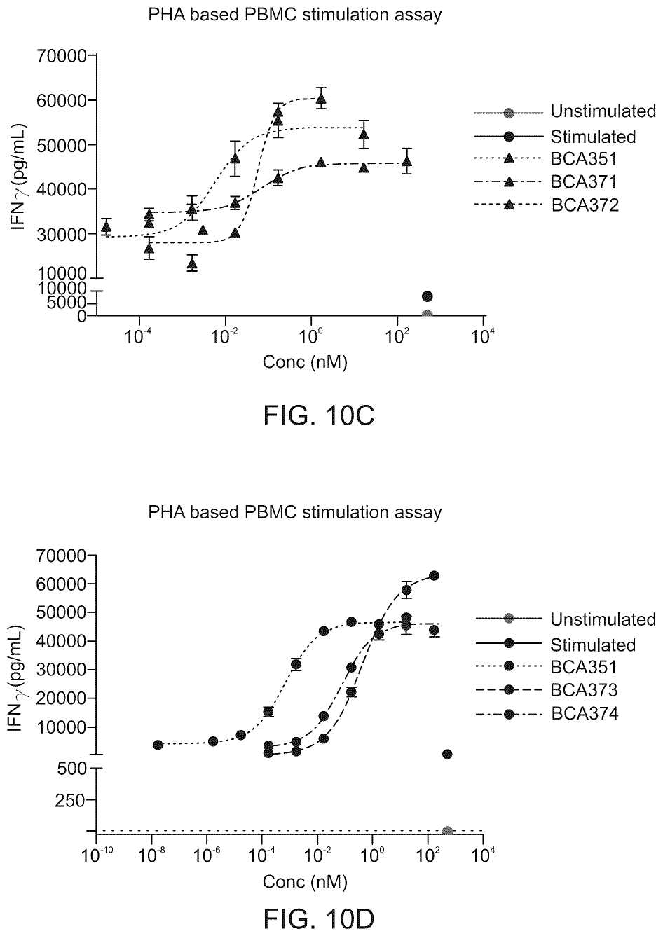

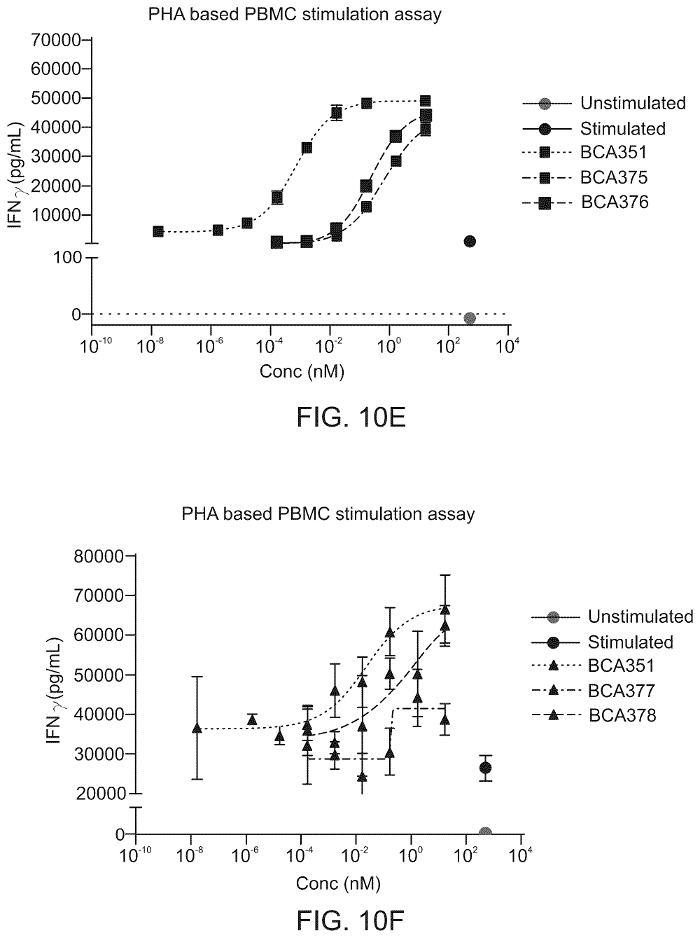

IL-12, secreted by antigen presenting cells, stimulates the effector functions of activated T-lymphocytes by induction of IFN-γ through phosphorylation of STAT4 potentiating anti-tumor immune response (see, e.g., Tugues, S et al. “New insights into IL-12-mediated tumor suppression.” Cell death and differentiation vol. 22, 2 (2015): 237-46. doi:10.1038/cdd.2014.134). The level of IFN-γ released by T-cells stimulated in the presence of each of the hIL-12p40 variants was evaluated. Briefly, hPBMCs were seeded at 0.1 million cells/well in a 96 well plate and stimulated overnight with PHA (1 μg/ml) in a volume of 100 μl. The following day, PHA was added again at the same concentration along with titrating concentration of the indicated hIL-12p40 variant. The cells were subsequently incubated at 37° C. in a 5% CO₂ incubator for ˜48 hours. IFN-γ was measured in the culture supernatant by a standard IFN-γ ELISA (FIGS. 10A-10F). As shown in FIG. 10A, the hIL-12p40 variant BCA356 is highly attenuated in T-cell activation compared to wild type IL-12 (BCA351) and recombinant human IL-12 (rhIL-12).

### 6.6 Example 6. Evaluation of hIL-12p40 Variant Activity-IFN-γ Release by hIL-2 Primed Human NK Cells

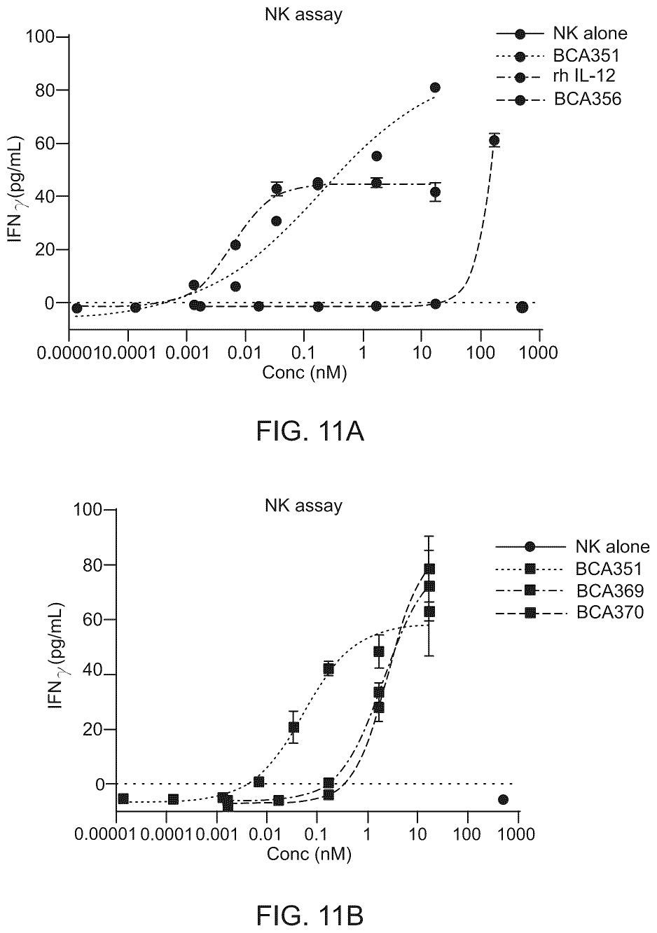

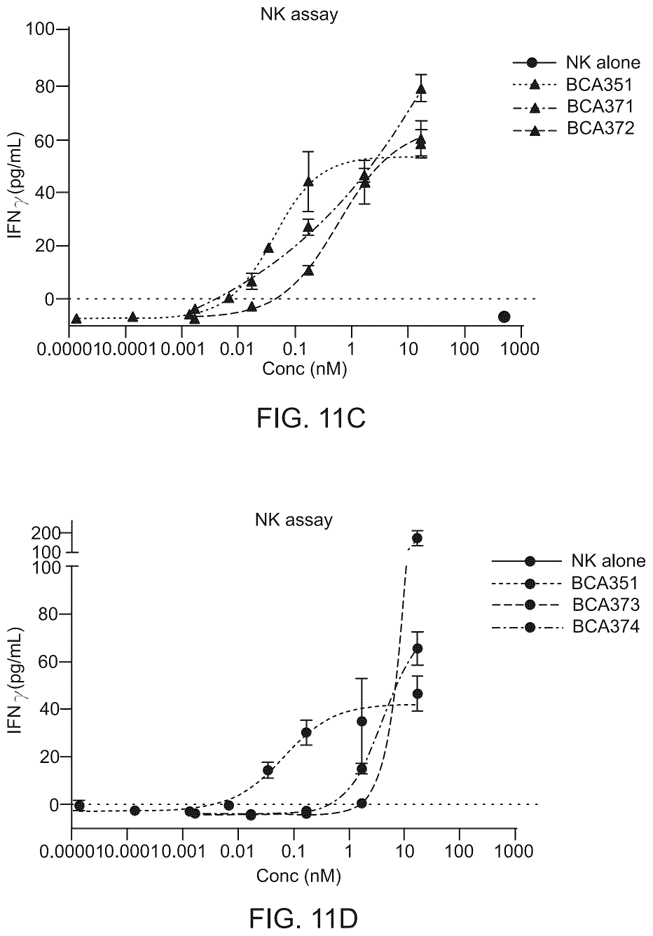

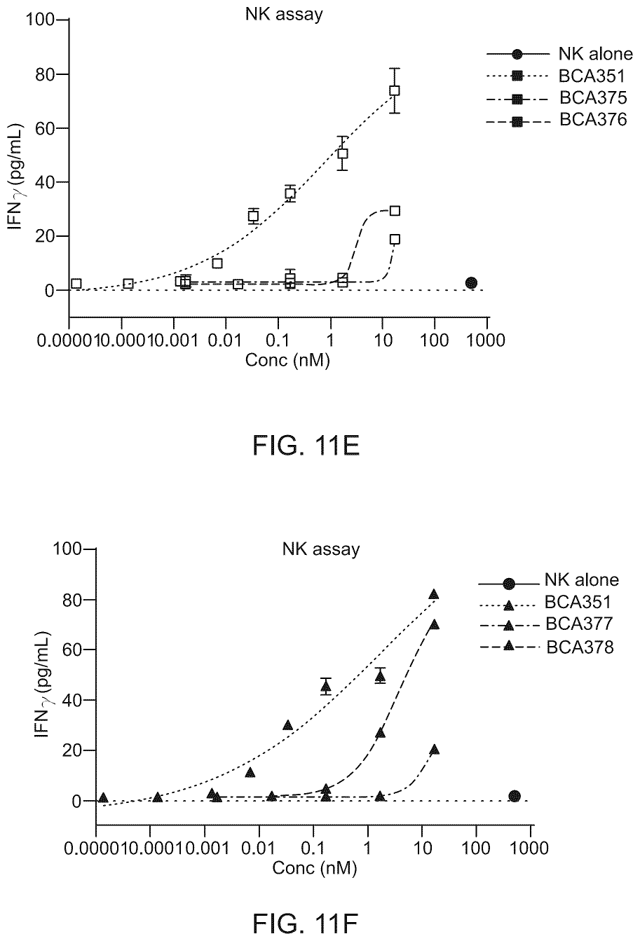

IL-12 stimulates NK cells to secrete IFN-γ and augments their proliferation and cytolytic activity (see, e.g., Glassman, Caleb R et al. “Structural basis for IL-12 and IL-23 receptor sharing reveals a gateway for shaping actions on T versus NK cells.” Cell vol. 184, 4 (2021): 983-999.e24. doi: 10.1016/j.cell.2021.01.018). To evaluate the level of IFN-γ released from NK cells, hPBMCs were thawed in RPMI 1640 containing 10% HI-FBS and 100 IU/ml hIL-2. After overnight incubation the cells were harvested, and the NK cells were enriched by negative selection. NK cells were seeded as 50,000 cells/well along with titrating concentration of hIL-12 and incubated at 37° C. in a 5% CO₂ for ˜48 hours. IFN-γ was measured in the culture supernatant by IFN-γ a standard IFN-γ ELISA (FIGS. 11A-11B). As shown in FIG. 11A, the hIL-12p40 variant BCA356 is highly attenuated in NK cell activation compared to wild type IL-12 (BCA351) and rhIL-12.

### 6.7 Example 7. Summary hIL-12p40 Variant Activity

The data presented in Examples 3-7 and summarized in Table 27 shows that hIL-12p40 variant BCA356 is ˜7000× attenuated compared to wild type IL-12 (Example 4) and highly attenuated in NK (Example 6) and T cell (Example 5) activation. However, hIL-12p40 variants containing single or double amino acid substitutions were not similarly attenuated (see, e.g., Table 27). Hence, the unique functional attributes of the hIL-12p40 variant BCA356 are due to the unique combination of all the three substitutions W37A, F82A and K219A.

TABLE 27
Summary of Functional Analysis of hIL-12p40 Variants

| | | | IFNγ | IFNγ |
|---|---|---|---|---|
| | | HEK-IL12 | release by | release |
| | | reporter | NK cells | by PHA |
| Des- | Amino Acid | assay | *Level of | stimulated |
| cription | Substitution(s) | Attenuation | attenuation | PBMC |
| BCA371 | K217A | 2 | + | + |
| BCA372 | K219A | 2 | + | + |
| BCA378 | K217A, K219A | 5 | ++ | + |
| BCA369 | W37A | 7.6 | ++ | + |
| BCA370 | F82A | 18 | ++ | + |
| BCA374 | W37A, K217A | 43 | ++ | ++ |
| BCA373 | W37A, F82A | 207 | ++ | ++ |
| BCA376 | F82A, K217A | 214 | +++ | ++ |
| BCA375 | W37A, K219A | 319 | +++ | ++ |
| BCA377 | F82A, K219A | 460 | +++ | +++ |
| BCA356 | W37A, F82A, | 7300 | +++ | +++ |
| | K219A | | | |

*Qualitative ranking done based on shifts in curves where +, ++, +++ represents low, mid and high attenuation compared to wild type hIL-12.

### 6.8 Example 8. Anti-CAIX hIL-12 Variant Fusion Proteins Exhibit Attenuated Activity In Vitro

The following anti-hCAIX hIL-12 fusion proteins were generated according to standard methods. Briefly, the different polypeptide sequence of each hIL-12p40 variant was codon optimized to obtain a DNA polynucleotide counterpart that was cloned into a suitable mammalian expression plasmid. The respective plasmids were transiently co-transfected into Expi-CHO-S cells. The transfected cells were grown for 7-10 days with appropriate feeding as per the manufacturer's protocol. The supernatants of the cell cultures were harvested and the hIL-12p40 proteins purified using Protein A and SEC affinity columns. The purified proteins were eluted in suitable buffer and subsequently diluted to working concentrations. In some experiments a reference construct was also assessed, including recombinant human IL-12 (rhIL-12) (R&D systems 10018-IL). The amino acid sequence of each of the anti-CAIX hIL-12 fusion proteins generated is set forth in Table 28, as well as control anti-diphtheria toxin (DT) hIL-12 antibody fusions. The signal sequence of each of the polypeptides is underlined.

TABLE 28
The Amino Acid Sequence of Anti-hCAIX hIL-12Fusion Proteins

| | | | SEQ |
|---|---|---|---|
| Description | | Amino Acid Sequence | ID NO |
| BCA313 | Fc Knob- | MMSFVSLLLVGILFHATQAEPKSCDKTHTCPPCPAPELLGGPSV | 386 |
| (Fc-IL-12) | hIL-12p40 | FLFPPKPKDTLMISRTPEVTCVVVDVSHEDPEVKENWYVDGVEV | |
| | | HNAKTKPREEQYNSTYRVVSVLTVLHQDWLNGKEYKCKVSNKAL | |
| | | PAPIEKTISKAKGQPREPQVYTLPPSRDELTKNQVSLWCLVKGF | |
| | | YPSDIAVEWESNGQPENNYKTTPPVLDSDGSFFLYSKLTVDKSR | |
| | | WQQGNVFSCSVMHEALHNHYTQKSLSLSPGKGGGGSGGGGSGGG | |
| | | GSIWELKKDVYVVELDWYPDAPGEMVVLTCDTPEEDGITWTLDQ | |
| | | SSEVLGSGKTLTIQVKKFGDAGQYTCHKGGEVLSHSLLLLHKKE | |
| | | DGIWSTDILKDQEEPKNKTELRCEAKNYSGRFTCWWLTTISTDL | |
| | | TFSVKSSRGSSDPQGVTCGAATLSAERVRGDNKEYEYSVECQED | |
| | | SACPAAEESLPIEVMVDAVHKLKYENYTSSFFIRDIIKPDPPKN | |
| | | LQLKPLKNSRQVEVSWEYPDTWSTPHSYFSLTFCVQVQGKSKRE | |
| | | KEDRVFTDKTSATVICRKNASISVRAQDRYYSSSWSEWASVPCS | |
| | Fc Hole- | MMSFVSLLLVGILFHATQAEPKSCDKTHTCPPCPAPELLGGPSV | 387 |
| | hIL-12p35 | FLFPPKPKDTLMISRTPEVTCVVVDVSHEDPEVKENWYVDGVEV | |
| | | HNAKTKPREEQYNSTYRVVSVLTVLHQDWLNGKEYKCKVSNKAL | |
| | | PAPIEKTISKAKGQPREPQVYTLPPSRDELTKNQVSLSCAVKGF | |
| | | YPSDIAVEWESNGQPENNYKTTPPVLDSDGSFFLVSKLTVDKSR | |
| | | WQQGNVFSCSVMHEALHNHYTQKSLSLSPGKGGGGSGGGGSGGG | |
| | | GSRNLPVATPDPGMFPCLHHSQNLLRAVSNMLQKARQTLEFYPC | |
| | | TSEEIDHEDITKDKTSTVEACLPLELTKNESCLNSRETSFITNG | |
| | | SCLASRKTSFMMALCLSSIYEDLKMYQVEFKTMNAKLLMDPKRQ | |
| | | IFLDQNMLAVIDELMQALNENSETVPQKSSLEEPDFYKTKIKLC | |
| | | ILLHAFRIRAVTIDRVMSYLNAS | |
| BCA350 | Anti-CAIX | MMSFVSLLLVGILFHATQAEVRLVESGGGLVKPGGSLRLSCAAS | 388 |
| (Humanized | HC Knob | GFTFSNYYMSWIRQAPEKRLELVSAINSDGGITYYLDTVKGRFT | |
| anti-CAIX) | | ISRDNAKNSLYLQMNSLRAEDTALFYCARHRSGYFSMDYWGQGT | |
| | | SVTVSSASTKGPSVFPLAPSSKSTSGGTAALGCLVKDYFPEPVT | |
| | | VSWNSGALTSGVHTFPAVLQSSGLYSLSSVVTVPSSSLGTQTYI | |
| | | CNVNHKPSNTKVDKKVEPKSCDKTHTCPPCPAPELLGGPSVELF | |
| | | PPKPKDTLMISRTPEVTCVVVDVSHEDPEVKENWYVDGVEVHNA | |
| | | KTKPREEQYNSTYRVVSVLTVLHQDWLNGKEYKCKVSNKALPAP | |
| | | IEKTISKAKGQPREPQVYTLPPSRDELTKNQVSLWCLVKGFYPS | |
| | | DIAVEWESNGQPENNYKTTPPVLDSDGSFFLYSKLTVDKSRWQQ | |
| | | GNVFSCSVMHEALHNHYTQKSLSLSPGK | |
| | Anti-CAIX | MMSFVSLLLVGILFHATQAEVRLVESGGGLVKPGGSLRLSCAAS | 389 |
| | HC Hole | GFTFSNYYMSWIRQAPEKRLELVSAINSDGGITYYLDTVKGRET | |
| | | ISRDNAKNSLYLQMNSLRAEDTALFYCARHRSGYFSMDYWGQGT | |
| | | SVTVSSASTKGPSVFPLAPSSKSTSGGTAALGCLVKDYFPEPVT | |
| | | VSWNSGALTSGVHTFPAVLQSSGLYSLSSVVTVPSSSLGTQTYI | |
| | | CNVNHKPSNTKVDKKVEPKSCDKTHTCPPCPAPELLGGPSVELE | |
| | | PPKPKDTLMISRTPEVTCVVVDVSHEDPEVKENWYVDGVEVHNA | |
| | | KTKPREEQYNSTYRVVSVLTVLHQDWLNGKEYKCKVSNKALPAP | |
| | | IEKTISKAKGQPREPQVYTLPPSRDELTKNQVSLSCAVKGFYPS | |
| | | DIAVEWESNGQPENNYKTTPPVLDSDGSFFLVSKLTVDKSRWQQ | |
| | | GNVFSCSVMHEALHNHYTQKSLSLSPGK | |
| | Anti-CAIX | MMSFVSLLLVGILFHATQAEIVMTQSPATLSVSPGERATLSCKA | 372 |
| | LC | SQNVVSAVAWYQQKPGQSPRLLIYSASNRYTGIPARESGSGSGT | |
| | | EFTLTISSLQSEDFAAYYCQQYRNYPWTFGGGTKVEIKRTVAAP | |
| | | SVFIFPPSDEQLKSGTASVVCLLNNFYPREAKVQWKVDNALQSG | |
| | | NSQESVTEQDSKDSTYSLSSTLTLSKADYEKHKVYACEVTHQGL | |
| | | SSPVTKSENRGEC | |
| BCA351 | Anti-CAIX | MMSFVSLLLVGILFHATQAEVRLVESGGGLVKPGGSLRLSCAAS | 383 |
| (Wild | HC Knob- | GFTFSNYYMSWIRQAPEKRLELVSAINSDGGITYYLDTVKGRFT | |
| type) | hIL-12p40 | ISRDNAKNSLYLQMNSLRAEDTALFYCARHRSGYFSMDYWGQGT | |
| | | SVTVSSASTKGPSVFPLAPSSKSTSGGTAALGCLVKDYFPEPVT | |
| | | VSWNSGALTSGVHTFPAVLQSSGLYSLSSVVTVPSSSLGTQTYI | |
| | | CNVNHKPSNTKVDKKVEPKSCDKTHTCPPCPAPELLGGPSVELE | |
| | | PPKPKDTLMISRTPEVTCVVVDVSHEDPEVKENWYVDGVEVHNA | |
| | | KTKPREEQYNSTYRVVSVLTVLHQDWLNGKEYKCKVSNKALPAP | |
| | | IEKTISKAKGQPREPQVYTLPPSRDELTKNQVSLWCLVKGFYPS | |
| | | DIAVEWESNGQPENNYKTTPPVLDSDGSFFLYSKLTVDKSRWQQ | |
| | | GNVFSCSVMHEALHNHYTQKSLSLSPGKGGGGSGGGGSGGGGSI | |
| | | WELKKDVYVVELDWYPDAPGEMVVLTCDTPEEDGITWTLDQSSE | |
| | | VLGSGKTLTIQVKEFGDAGQYTCHKGGEVLSHSLLLLHKKEDGI | |
| | | WSTDILKDQKEPKNKTFLRCEAKNYSGRFTCWWLTTISTDLTES | |
| | | VKSSRGSSDPQGVTCGAATLSAERVRGDNKEYEYSVECQEDSAC | |
| | | PAAEESLPIEVMVDAVHKLKYENYTSSFFIRDIIKPDPPKNLQL | |
| | | KPLKNSRQVEVSWEYPDTWSTPHSYFSLTFCVQVQGKSKREKKD | |
| | | RVFTDKTSATVICRKNASISVRAQDRYYSSSWSEWASVPCS | |
| | Anti-CAIX | MMSFVSLLLVGILFHATQAEVRLVESGGGLVKPGGSLRLSCAAS | 371 |
| | HC Hole- | GFTFSNYYMSWIRQAPEKRLELVSAINSDGGITYYLDTVKGRFT | |
| | hIL-12p35 | ISRDNAKNSLYLQMNSLRAEDTALFYCARHRSGYFSMDYWGQGT | |
| | | SVTVSSASTKGPSVFPLAPSSKSTSGGTAALGCLVKDYFPEPVT | |
| | | VSWNSGALTSGVHTFPAVLQSSGLYSLSSVVTVPSSSLGTQTYI | |
| | | CNVNHKPSNTKVDKKVEPKSCDKTHTCPPCPAPELLGGPSVELE | |
| | | PPKPKDTLMISRTPEVTCVVVDVSHEDPEVKENWYVDGVEVHNA | |
| | | KTKPREEQYNSTYRVVSVLTVLHQDWLNGKEYKCKVSNKALPAP | |
| | | IEKTISKAKGQPREPQVYTLPPSRDELTKNQVSLSCAVKGFYPS | |
| | | DIAVEWESNGQPENNYKTTPPVLDSDGSFFLVSKLTVDKSRWQQ | |
| | | GNVFSCSVMHEALHNHYTQKSLSLSPGKGGGGSGGGGGGGGSR | |
| | | NLPVATPDPGMFPCLHHSQNLLRAVSNMLQKARQTLEFYPCTSE | |
| | | EIDHEDITKDKTSTVEACLPLELTKNESCLNSRETSFITNGSCL | |
| | | ASRKTSFMMALCLSSIYEDLKMYQVEFKTMNAKLLMDPKRQIFL | |
| | | DQNMLAVIDELMQALNENSETVPQKSSLEEPDFYKTKIKLCILL | |
| | | HAFRIRAVTIDRVMSYLNAS | |
| | Anti-CAIX | MMSFVSLLLVGILFHATQAEIVMTQSPATLSVSPGERATLSCKA | 372 |
| | LC | SQNVVSAVAWYQQKPGQSPRLLIYSASNRYTGIPARFSGSGSGT | |
| | | EFTLTISSLQSEDFAAYYCQQYRNYPWTFGGGTKVEIKRTVAAP | |
| | | SVFIFPPSDEQLKSGTASVVCLLNNFYPREAKVQWKVDNALQSG | |
| | | NSQESVTEQDSKDSTYSLSSTLTLSKADYEKHKVYACEVTHQGL | |
| | | SSPVTKSENRGEC | |
| BCA356 | Anti-CAIX | MMSFVSLLLVGILFHATQAEVRLVESGGGLVKPGGSLRLSCAAS | 382 |
| | HC Knob- | GFTFSNYYMSWIRQAPEKRLELVSAINSDGGITYYLDTVKGRFT | |
| | hIL-12p40 | ISRDNAKNSLYLQMNSLRAEDTALFYCARHRSGYFSMDYWGQGT | |
| | | SVTVSSASTKGPSVFPLAPSSKSTSGGTAALGCLVKDYFPEPVT | |
| | | VSWNSGALTSGVHTFPAVLQSSGLYSLSSVVTVPSSSLGTQTYI | |
| | | CNVNHKPSNTKVDKKVEPKSCDKTHTCPPCPAPELLGGPSVELE | |
| | | PPKPKDTLMISRTPEVTCVVVDVSHEDPEVKENWYVDGVEVHNA | |
| | | KTKPREEQYNSTYRVVSVLTVLHQDWLNGKEYKCKVSNKALPAP | |
| | | IEKTISKAKGQPREPQVYTLPPSRDELTKNQVSLWCLVKGFYPS | |
| | | DIAVEWESNGQPENNYKTTPPVLDSDGSFFLYSKLTVDKSRWQQ | |
| | | GNVESCSVMHEALHNHYTQKSLSLSPGKGGGGSGGGGSGGGGSI | |
| | | WELKKDVYVVELDAYPDAPGEMVVLTCDTPEEDGITWTLDQSSE | |
| | | VLGSGKTLTIQVKEAGDAGQYTCHKGGEVLSHSLLLLHKKEDGI | |
| | | WSTDILKDQKEPKNKTFLRCEAKNYSGRFTCWWLTTISTDLTES | |
| | | VKSSRGSSDPQGVTCGAATLSAERVRGDNKEYEYSVECQEDSAC | |
| | | PAAEESLPIEVMVDAVHKLAYENYTSSFFIRDIIKPDPPKNLQL | |
| | | KPLKNSRQVEVSWEYPDTWSTPHSYFSLTFCVQVQGKSKREKKD | |
| | | RVFTDKTSATVICRKNASISVRAQDRYYSSSWSEWASVPCS | |
| | Anti-CAIX | MMSFVSLLLVGILFHATQAEVRLVESGGGLVKPGGSLRLSCAAS | 371 |
| | HC Hole- | GFTFSNYYMSWIRQAPEKRLELVSAINSDGGITYYLDTVKGRET | |
| | hIL-12p35 | ISRDNAKNSLYLQMNSLRAEDTALFYCARHRSGYFSMDYWGQGT | |
| | | SVTVSSASTKGPSVFPLAPSSKSTSGGTAALGCLVKDYFPEPVT | |
| | | VSWNSGALTSGVHTFPAVLQSSGLYSLSSVVTVPSSSLGTQTYI | |
| | | CNVNHKPSNTKVDKKVEPKSCDKTHTCPPCPAPELLGGPSVELF | |
| | | PPKPKDTLMISRTPEVTCVVVDVSHEDPEVKENWYVDGVEVHNA | |
| | | KTKPREEQYNSTYRVVSVLTVLHQDWLNGKEYKCKVSNKALPAP | |
| | | IEKTISKAKGQPREPQVYTLPPSRDELTKNQVSLSCAVKGFYPS | |
| | | DIAVEWESNGQPENNYKTTPPVLDSDGSFFLVSKLTVDKSRWQQ | |
| | | GNVFSCSVMHEALHNHYTQKSLSLSPGKGGGGSGGGGSGGGGSR | |
| | | NLPVATPDPGMFPCLHHSQNLLRAVSNMLQKARQTLEFYPCTSE | |
| | | EIDHEDITKDKTSTVEACLPLELTKNESCLNSRETSFITNGSCL | |
| | | ASRKTSFMMALCLSSIYEDLKMYQVEFKTMNAKLLMDPKRQIFL | |
| | | DQNMLAVIDELMQALNENSETVPQKSSLEEPDFYKTKIKLCILL | |
| | | HAFRIRAVTIDRVMSYLNAS | |
| | Anti-CAIX | MMSFVSLLLVGILFHATQAEIVMTQSPATLSVSPGERATLSCKA | 372 |
| | LC | SQNVVSAVAWYQQKPGQSPRLLIYSASNRYTGIPARFSGSGSGT | |
| | | EFTLTISSLQSEDFAAYYCQQYRNYPWTFGGGTKVEIKRTVAAP | |
| | | SVFIFPPSDEQLKSGTASVVCLLNNFYPREAKVQWKVDNALQSG | |
| | | NSQESVTEQDSKDSTYSLSSTLTLSKADYEKHKVYACEVTHQGL | |
| | | SSPVTKSENRGEC | |
| BCA380 | Anti-DT HC | MMSFVSLLLVGILFHATQAQVQLQQSGPGPAKPSQTLSLTCAIS | 390 |
| | Knob-hIL- | GDSVSSDSAAWNWIRQSPSRGLEWLGRTYYRSTWYRDYAPSVKS | |
| | 12p40 | RITINPDTSKNQFSLQLNSVTPEDTAVYYCARDKDSFESNGSLY | |
| | | TAKKMGFDPWGQGTLVTVSSASTKGPSVFPLAPSSKSTSGGTAA | |
| | | LGCLVKDYFPEPVTVSWNSGALTSGVHTFPAVLQSSGLYSLSSV | |
| | | VTVPSSSLGTQTYICNVNHKPSNTKVDKKVEPKSCDKTHTCPPC | |
| | | PAPELLGGPSVFLFPPKPKDTLMISRTPEVTCVVVDVSHEDPEV | |
| | | KFNWYVDGVEVHNAKTKPREEQYNSTYRVVSVLTVLHQDWLNGK | |
| | | EYKCKVSNKALPAPIEKTISKAKGQPREPQVYTLPPSRDELTKN | |
| | | QVSLWCLVKGFYPSDIAVEWESNGQPENNYKTTPPVLDSDGSFF | |
| | | LYSKLTVDKSRWQQGNVFSCSVMHEALHNHYTQKSLSLSPGKGG | |
| | | GGSGGGGSGGGGSIWELKKDVYVVELDWYPDAPGEMVVLTCDTP | |
| | | EEDGITWTLDQSSEVLGSGKTLTIQVKEFGDAGQYTCHKGGEVL | |
| | | SHSLLLLHKKEDGIWSTDILKDQKEPKNKTFLRCEAKNYSGRFT | |
| | | CWWLTTISTDLTFSVKSSRGSSDPQGVTCGAATLSAERVRGDNK | |
| | | EYEYSVECQEDSACPAAEESLPIEVMVDAVHKLKYENYTSSFFI | |
| | | RDIIKPDPPKNLQLKPLKNSRQVEVSWEYPDTWSTPHSYFSLTE | |
| | | CVQVQGKSKREKKDRVFTDKTSATVICRKNASISVRAQDRYYSS | |
| | | SWSEWASVPCS | |
| | Anti-DT HC | MMSFVSLLLVGILFHATQAQVQLQQSGPGPAKPSQTLSLTCAIS | 391 |
| | Hole-hIL- | GDSVSSDSAAWNWIRQSPSRGLEWLGRTYYRSTWYRDYAPSVKS | |
| | 12p35 | RITINPDTSKNQFSLQLNSVTPEDTAVYYCARDKDSFESNGSLY | |
| | | TAKKMGFDPWGQGTLVTVSSASTKGPSVFPLAPSSKSTSGGTAA | |
| | | LGCLVKDYFPEPVTVSWNSGALTSGVHTFPAVLQSSGLYSLSSV | |
| | | VTVPSSSLGTQTYICNVNHKPSNTKVDKKVEPKSCDKTHTCPPC | |
| | | PAPELLGGPSVFLFPPKPKDTLMISRTPEVTCVVVDVSHEDPEV | |
| | | KFNWYVDGVEVHNAKTKPREEQYNSTYRVVSVLTVLHQDWLNGK | |
| | | EYKCKVSNKALPAPIEKTISKAKGQPREPQVYTLPPSRDELTKN | |
| | | QVSLSCAVKGFYPSDIAVEWESNGQPENNYKTTPPVLDSDGSFF | |
| | | LVSKLTVDKSRWQQGNVFSCSVMHEALHNHYTQKSLSLSPGKGG | |
| | | GGSGGGGSGGGGSRNLPVATPDPGMFPCLHHSQNLLRAVSNMLQ | |
| | | KARQTLEFYPCTSEEIDHEDITKDKTSTVEACLPLELTKNESCL | |
| | | NSRETSFITNGSCLASRKTSEMMALCLSSIYEDLKMYQVEFKTM | |
| | | NAKLLMDPKRQIFLDQNMLAVIDELMQALNENSETVPQKSSLEE | |
| | | PDFYKTKIKLCILLHAFRIRAVTIDRVMSYLNAS | |
| | Anti-DT LC | MMSFVSLLLVGILFHATQAETTLTQSPGTLSLSPGERATLSCRA | 392 |
| | | SQSVRSSYLAWYQQKPGQAPRLLIYGASSRATGIPERFSGSGSG | |
| | | TDFTLTISRLEPEDFAVYYCQQYGSSPITFGQGTRLEIKRTVAA | |
| | | PSVFIFPPSDEQLKSGTASVVCLLNNFYPREAKVQWKVDNALQS | |
| | | GNSQESVTEQDSKDSTYSLSSTLTLSKADYEKHKVYACEVTHQG | |
| | | LSSPVTKSENRGEC | |
| BCA382 | Anti-DT HC | MMSFVSLLLVGILFHATQAQVQLQQSGPGPAKPSQTLSLTCAIS | 393 |
| | Knob-hIL- | GDSVSSDSAAWNWIRQSPSRGLEWLGRTYYRSTWYRDYAPSVKS | |
| | 12p40 | RITINPDTSKNQFSLQLNSVTPEDTAVYYCARDKDSFESNGSLY | |
| | | TAKKMGFDPWGQGTLVTVSSASTKGPSVFPLAPSSKSTSGGTAA | |
| | | LGCLVKDYFPEPVTVSWNSGALTSGVHTFPAVLQSSGLYSLSSV | |
| | | VTVPSSSLGTQTYICNVNHKPSNTKVDKKVEPKSCDKTHTCPPC | |
| | | PAPELLGGPSVFLFPPKPKDTLMISRTPEVTCVVVDVSHEDPEV | |
| | | KFNWYVDGVEVHNAKTKPREEQYNSTYRVVSVLTVLHQDWLNGK | |
| | | EYKCKVSNKALPAPIEKTISKAKGQPREPQVYTLPPSRDELTKN | |
| | | QVSLWCLVKGFYPSDIAVEWESNGQPENNYKTTPPVLDSDGSFF | |
| | | LYSKLTVDKSRWQQGNVFSCSVMHEALHNHYTQKSLSLSPGKGG | |
| | | GGSGGGGSGGGGSIWELKKDVYVVELDAYPDAPGEMVVLTCDTP | |
| | | EEDGITWTLDQSSEVLGSGKTLTIQVKEAGDAGQYTCHKGGEVL | |
| | | SHSLLLLHKKEDGIWSTDILKDQKEPKNKTFLRCEAKNYSGRET | |
| | | CWWLTTISTDLTFSVKSSRGSSDPQGVTCGAATLSAERVRGDNK | |
| | | EYEYSVECQEDSACPAAEESLPIEVMVDAVHKLAYENYTSSFFI | |
| | | RDIIKPDPPKNLQLKPLKNSRQVEVSWEYPDTWSTPHSYFSLTF | |
| | | CVQVQGKSKREKKDRVFTDKTSATVICRKNASISVRAQDRYYSS | |
| | | SWSEWASVPCS | |
| | Anti-DT HC | MMSFVSLLLVGILFHATQAQVQLQQSGPGPAKPSQTLSLTCAIS | 391 |
| | Hole-hIL- | GDSVSSDSAAWNWIRQSPSRGLEWLGRTYYRSTWYRDYAPSVKS | |
| | 12p35 | RITINPDTSKNQFSLQLNSVTPEDTAVYYCARDKDSFESNGSLY | |
| | | TAKKMGFDPWGQGTLVTVSSASTKGPSVFPLAPSSKSTSGGTAA | |
| | | LGCLVKDYFPEPVTVSWNSGALTSGVHTFPAVLQSSGLYSLSSV | |
| | | VTVPSSSLGTQTYICNVNHKPSNTKVDKKVEPKSCDKTHTCPPC | |
| | | PAPELLGGPSVFLFPPKPKDTLMISRTPEVTCVVVDVSHEDPEV | |
| | | KFNWYVDGVEVHNAKTKPREEQYNSTYRVVSVLTVLHQDWLNGK | |
| | | EYKCKVSNKALPAPIEKTISKAKGQPREPQVYTLPPSRDELTKN | |
| | | QVSLSCAVKGFYPSDIAVEWESNGQPENNYKTTPPVLDSDGSFF | |
| | | LVSKLTVDKSRWQQGNVFSCSVMHEALHNHYTQKSLSLSPGKGG | |
| | | GGSGGGGSGGGGSRNLPVATPDPGMFPCLHHSQNLLRAVSNMLQ | |
| | | KARQTLEFYPCTSEEIDHEDITKDKTSTVEACLPLELTKNESCL | |
| | | NSRETSFITNGSCLASRKTSEMMALCLSSIYEDLKMYQVEFKTM | |
| | | NAKLLMDPKRQIFLDQNMLAVIDELMQALNENSETVPQKSSLEE | |
| | | PDFYKTKIKLCILLHAFRIRAVTIDRVMSYLNAS | |
| | Anti-DT LC | MMSFVSLLLVGILFHATQAETTLTQSPGTLSLSPGERATLSCRA | 392 |
| | | SQSVRSSYLAWYQQKPGQAPRLLIYGASSRATGIPERFSGSGSG | |
| | | TDFTLTISRLEPEDFAVYYCQQYGSSPITFGQGTRLEIKRTVAA | |
| | | PSVFIFPPSDEQLKSGTASVVCLLNNFYPREAKVQWKVDNALQS | |
| | | GNSQESVTEQDSKDSTYSLSSTLTLSKADYEKHKVYACEVTHQG | |
| | | LSSPVTKSENRGEC | |

The biological activity of each of the proteins in Table 28 was evaluated using a HEK-IL-12 reporter assay according to manufacturer's instructions (InvivoGen). Briefly, HEK-Blue™ IL-12 cells (InvivoGen (hkb-IL-12)) are HEK cells stably transfected with polynucleotides encoding the human IL-12 receptor (hIL-12R) and the hIL-12 signaling pathway that express a STAT4-inducible SEAP reporter gene. The binding of IL-12 to the IL-12R on the surface of HEK-Blue™ IL-12 cells triggers a signaling cascade leading to the activation (and phosphorylation) of STAT-4 with the subsequent production of SEAP. Detection of SEAP in the supernatant of HEK-Blue™ IL-12 cells can be readily using QUANTI-Blue™ Solution. Here, approximately 50,000 HEK-Blue™ IL-12 cells (InvivoGen (hkb-IL-12)) were plated in a 96-well plate and incubated overnight (37° C., 5% CO₂) followed by treatment with the indicated construct for 18-24 hours. The IgG-Control construct was an anti-diphtheria toxin antibody SEAP was detected in the supernatant of the each of the cultures using QUANTI-Blue™ Solution according to manufacturer's instructions.

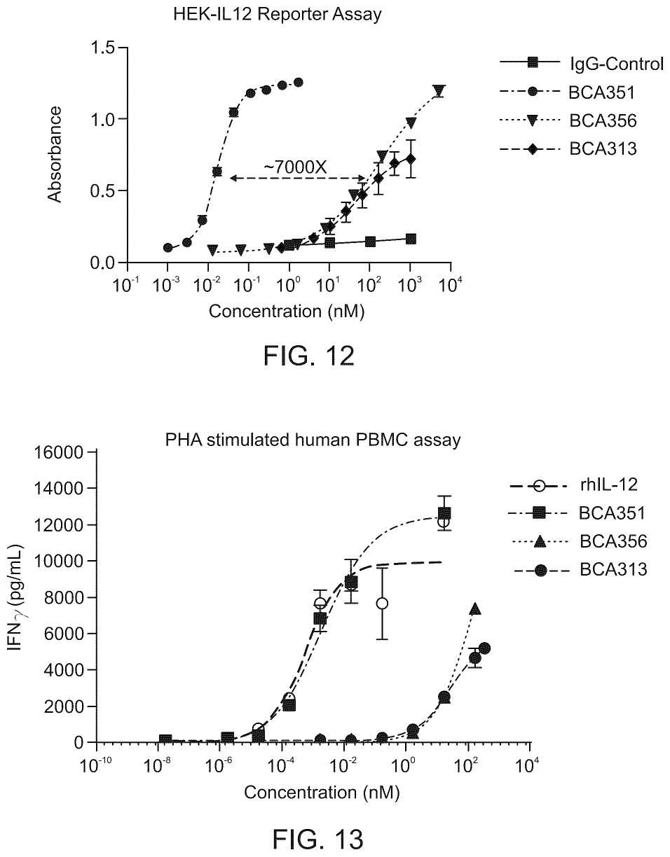

As shown, in FIG. 12, BCA356 IL-12 activity was attenuated by approximately 7000-fold compared to BCA351 (wild type IL-12 fusion). The reported value is an average of three independent experiments.

### 6.9 Example 9. Anti-CAIX hIL-12 Variant Fusion Proteins Mediate T-Cell Activation In Vitro

IL-12, secreted by antigen presenting cells, stimulates the effector functions of activated T-lymphocytes by induction of IFN-γ through phosphorylation of STAT4 potentiating anti-tumor immune response (see, e.g., Tugues, S et al. “New insights into IL-12-mediated tumor suppression.” Cell death and differentiation vol. 22, 2 (2015): 237-46. doi:10.1038/cdd.2014.134). The level of IFN-γ released by T-cells stimulated in the presence of each of the anti-CAIX hIL-12 fusion proteins was evaluated. Briefly, hPBMCs were seeded at 0.1 million cells/well in a 96 well plate and stimulated overnight with PHA (1 μg/ml) in a volume of 100 μl. Subsequently, PHA was again added at the same concentration of lug/ml along with titrating concentration of the indicated construct and incubated at 37° C. in a 5% CO₂ for ˜48 hours. The level of IFN-γ was measured in the culture supernatant by a standard IFN-γ ELISA. As shown in FIG. 13, in presence the anti-CAIX hIL-12 fusion protein BCA356 the level of IFN-γ released by activated T cells was lower than in the presence of recombinant hIL-12 and BCA351.

The binding of hIL-12 to its receptor complex stimulates phosphorylation of STAT4 (pSTAT) which translocates to the nucleus to promote IFN-γ gene transcription (see, e.g., Teng, Michele W L et al. “IL-12 and IL-23 cytokines: from discovery to targeted therapies for immune-mediated inflammatory diseases.” Nature medicine vol. 21, 7 (2015): 719-29. doi: 10.1038/nm.3895). The level of pSTAT4 in cells treated with each of the anti-CAIX IL-12 fusion proteins was evaluated. Briefly, hPBMCs were activated with plate coated anti-CD3 (2.5 μg/mL) and soluble anti-CD28 (5 μg/mL) antibodies for 48 h and rested overnight post activation. Post resting, the cells were stained with anti-human CD45RA, CD4, CD8, CD25 antibodies for 30 mins followed by incubation with rhIL-12, BCA351, BCA356, or BCA313 at the indicated concentration for 20 mins. The cells were then fixed, permeabilized, and stained with an anti-pSTAT4 antibody. The readout presented in FIG. 15 is the percent frequency of pSTAT4 expressing CD45RA CD8+CD25+ cells. As shown in FIG. 15, the level of pSTAT4 expressed by activated CD8+ T-cells in presence of BCA356 was lower than in the presence of recombinant hIL-12 or BCA351.

### 6.10 Example 10. Anti-CAIX hIL-12 Variant Fusion Proteins Mediate NK Cell Activation In Vitro

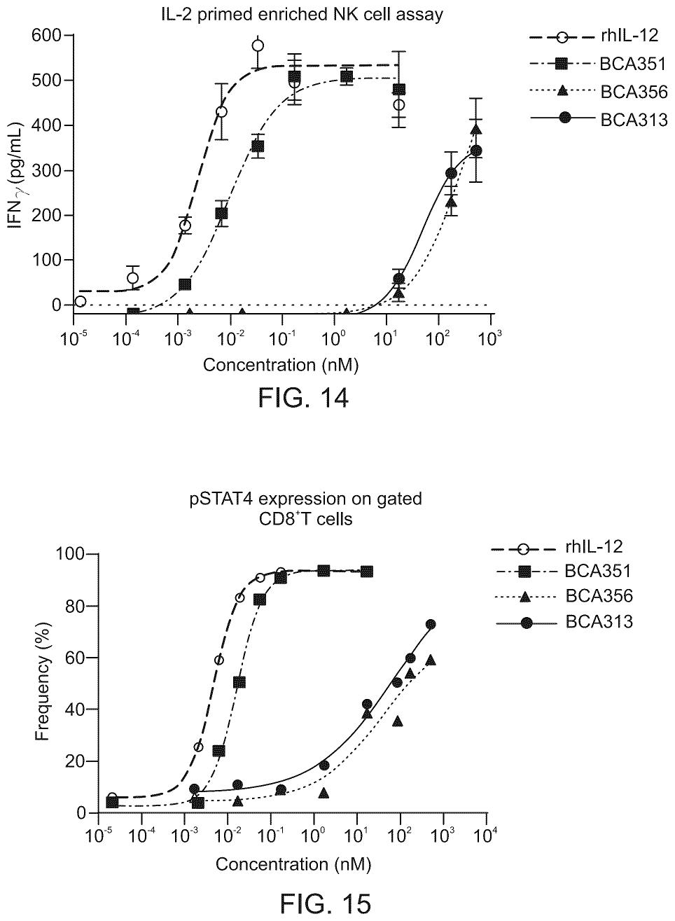

IL-12 activates NK cells to secrete IFN-γ and augments their proliferation and cytolytic activity (see, e.g., Glassman, Caleb R et al. “Structural basis for IL-12 and IL-23 receptor sharing reveals a gateway for shaping actions on T versus NK cells.” Cell vol. 184, 4 (2021): 983-999.e24. doi: 10.1016/j.cell.2021.01.018). The level of IFN-γ released from NK cells treated with each of the anti-CAIX hIL-12 fusion proteins was assessed. Briefly, hPBMCs were cultured in RPMI 1640 containing 10% HI-FBS and 100 IU/ml IL-2. After overnight incubation (37° C., 5% CO₂) the cells were harvested, and the NK cells were enriched by negative selection. NK cells were seeded at 50,000 cells/well in a 96 well plate along with titrating concentration of the indicated construct. The cells were incubated at 37° C. 5% CO₂ for ˜48 hours. The level of IFN-γ was measured in the culture supernatant by a standard IFN-γ ELISA. As shown in FIG. 14, in presence of the anti-CAIX hIL-12 fusion protein BCA356 the level of IFN-γ released by activated T cells was lower than in the presence of recombinant hIL-12 and BCA351.

### 6.11 Example 11. Anti-CAIX hIL-12 Variant Fusion Proteins Exhibit Substantial Cytotoxicity and Reduced Cytokine Release In Vitro

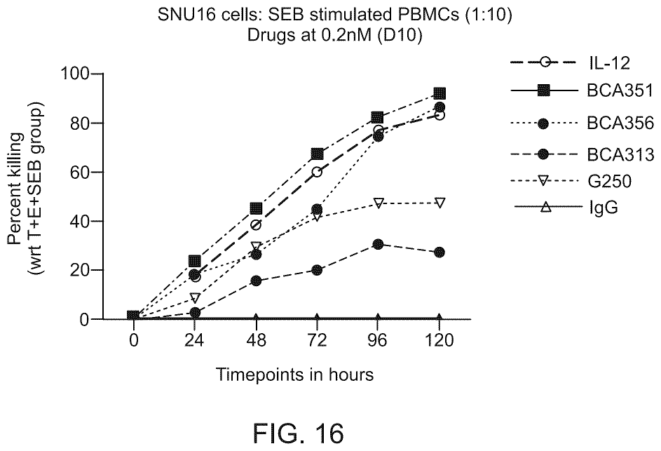

Real time tumor killing by the anti-CAIX hIL-12 fusion protein BCA356 was evaluated using a co-culture assay comprising gastric cancer SNU16 cells and activated hPBMC. Briefly, CAIX overexpressing SNU16 cancer cells were labelled with nuclight green using lentiviral transduction. Labelled SNU16 cells were seeded at 5,000 cells/well in a 96 well plate along with 50,000 cells/well of hPBMCs at a target (T): effector (E) ratio of 1:10. hPBMC were activated with 0.5 pg/ml of SEB (Staphylococcus aureus enterotoxin B) along with titrating concentration of the indicated construct. The plates were placed in an IncuCyte S3® (Essen BioScience) incubator at 37° C. with 5% CO₂ and were imaged for 5 days (120 hours). An increase in fluorescent signal corresponds to target cell proliferation and a decrease in fluorescent signal corresponds to target cell death. % Killing was calculated as={(signal intensity at a specific time point with antibody/T+E+SEB)*100}. As shown in FIG. 16, the anti-CAIX hIL-12 fusion protein BCA356 showed similar cytotoxicity compared to the anti-CAIX hIL-12 fusion protein BCA351 suggesting that reduced T-cell activation by the anti-CAIX hIL-12 fusion protein BCA356 does not influence tumor cell killing activity. BCA356 showed improved cytotoxicity over BCA313, probably due to CA-IX targeting.

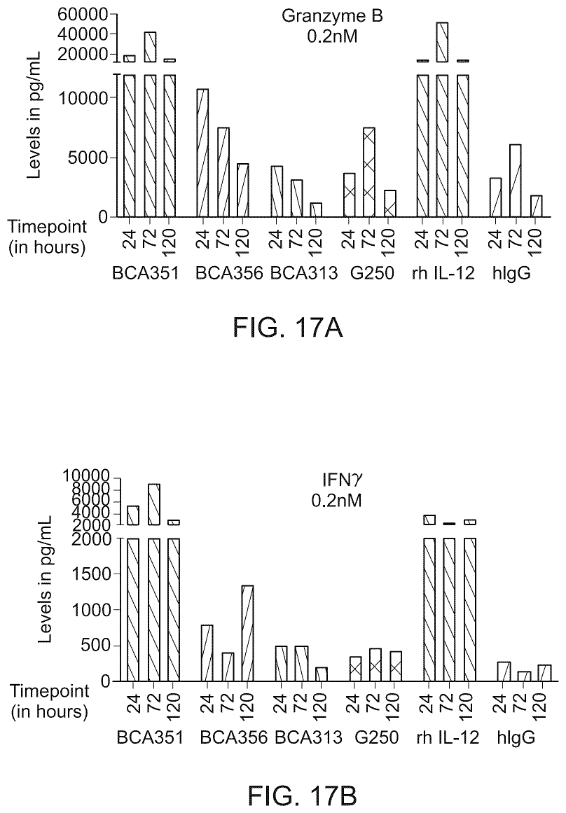

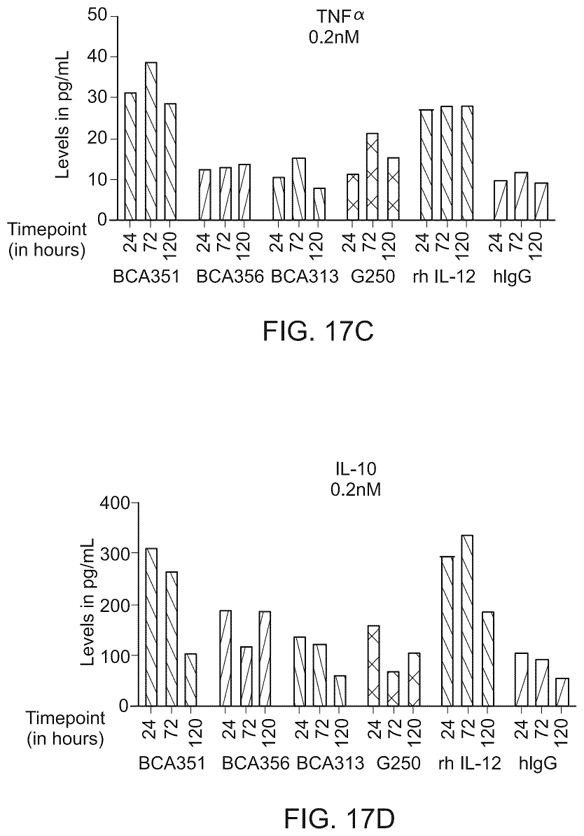

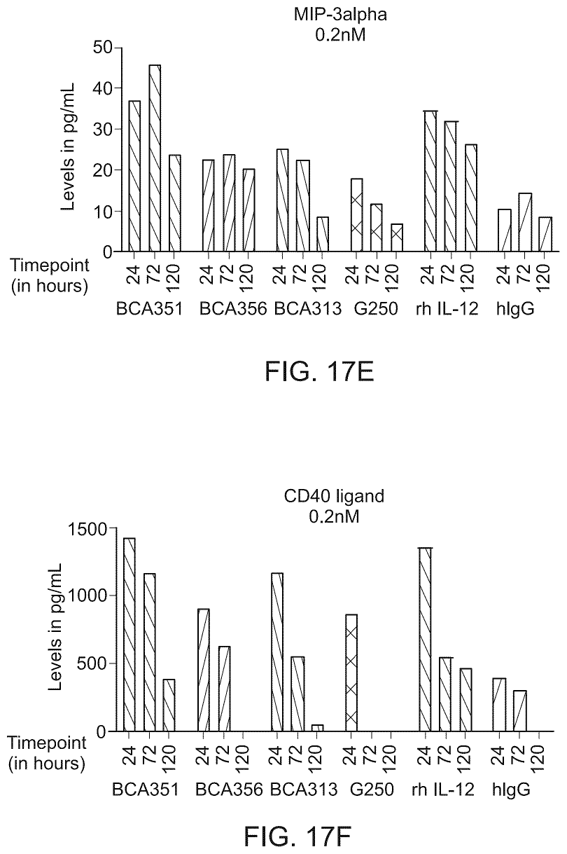

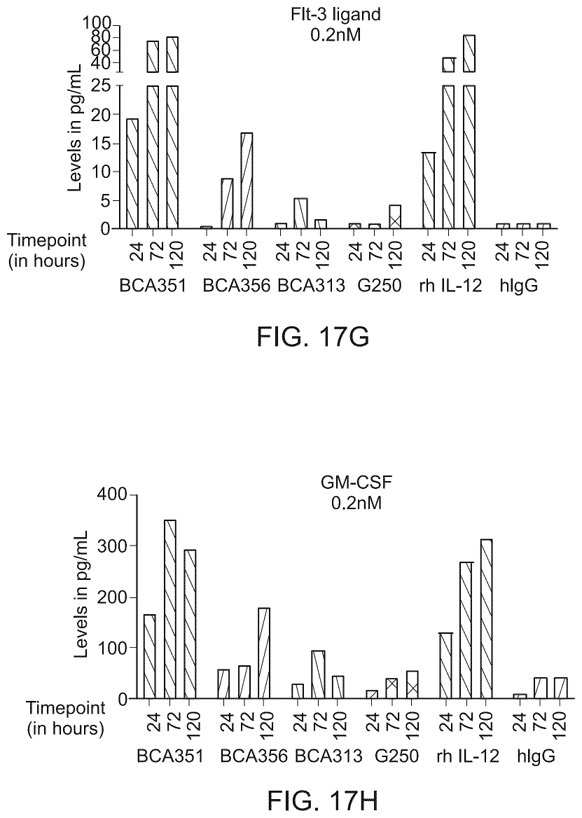

In addition, activated T cells secrete chemokines and cytokines that help in T-cell trafficking, activation, and anti-tumor immune response. A human cytokine luminex assay (44-plex kit) was used to evaluate cytokine production at 24 h, 72 h and 120 h post treatment with the indicated construct. Criteria used for selection of key cytokines was: 2-fold increase in BCA351 over Human IgG and >20% reduction in BCA356 over BCA351 at 24 h. Selected cytokines/chemokines at 24 h were compared across 72 h and 120 h timepoints. As shown in FIGS. 17A-H, the anti-CAIX hIL-12 fusion protein BCA356 showed limited release of Granzyme-B, IFN-γ, TNFα, IL-10, Flt3-L, GMCSF, MIP-3, and CD40-L compared to the anti-CAIX hIL-12 fusion protein BCA351 and rhIL-12.

### 6.12 Example 12. Anti-CAIX hIL-12 Variant Fusion Proteins Exhibit NK Cell Mediated Cytotoxicity of Target Cells In Vitro

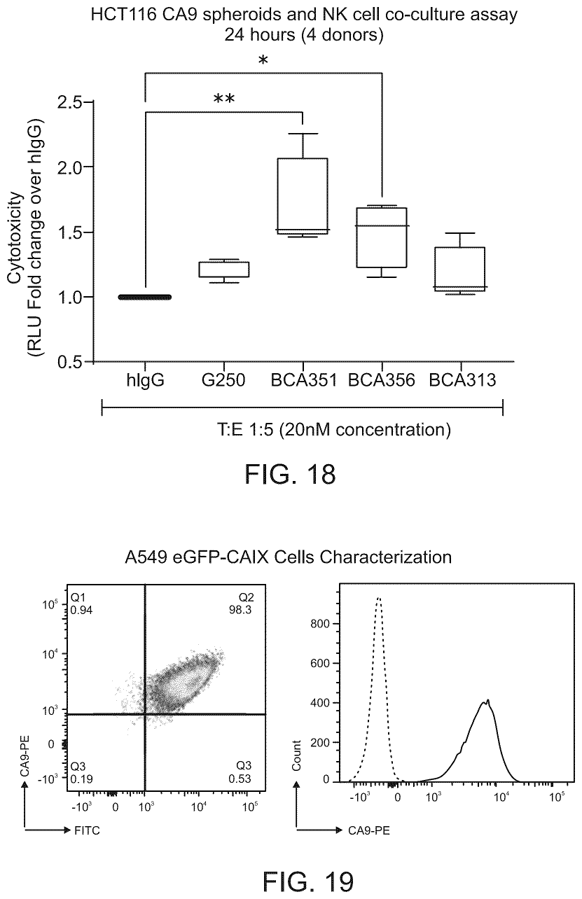

The ability of the anti-CAIX hIL-12 fusion protein BCA356 to potentiate NK cell-based tumor killing activity was evaluated. Briefly, HCT116 colon cancer cells were transfected with hCAIX and seeded at 5,000 cells/well in an ultra-low attachment 96 well plate and allowed to form 3D spheroids for 96 hours. These spheroids were treated with 20 nM concentration of the indicated construct and incubated for 1-2 hours, post which, IL-2 primed, and enriched NK cells were added at T: E ratio of 1:5. The abbreviation G250 indicates the anti-hCAIX antibody girentuximab. The cells (coculture) were incubated for 24 hours and tumor killing assessed using CytoToxGlo® reagent, which measures the proteases of dead cells. Cytotoxicity was calculated as fold change in relative luminescence intensity (RLU) of the construct over hIgG. As shown in FIG. 18, the anti-CAIX hIL-12 fusion protein BCA356 showed NK cell mediated killing of hCAIX overexpressing HCT116 cells, while the FcIL-12 fusion protein BCA313 did not, likely due to lack of CAIX targeting of the construct. BCA356 showed superior NK cell mediated killing of target cells than G250 due to the combination with IL-12v.

### 6.13 Example 13. Anti-CAIX hIL-12 Variant Fusion Proteins Exhibit Cytotoxicity In Vitro in Lung Cancer Model

The ability of the anti-CAIX hIL-12 fusion protein BCA356 to mediate cytotoxicity of the A549 lung cancer model was assessed. Briefly, A549 lung cancer cells were stably transfected with a plasmid encoding eGFP and a CAIX-eGFP fusion protein (eGFP fused to the C-terminus of CAIX). Single cell clones expressing eGFP-CAIX were selected based on puromycin resistance and characterized by flow cytometry. A representative histogram and dot plot showing the eGFP-CAIX expression are shown in FIG. 19.

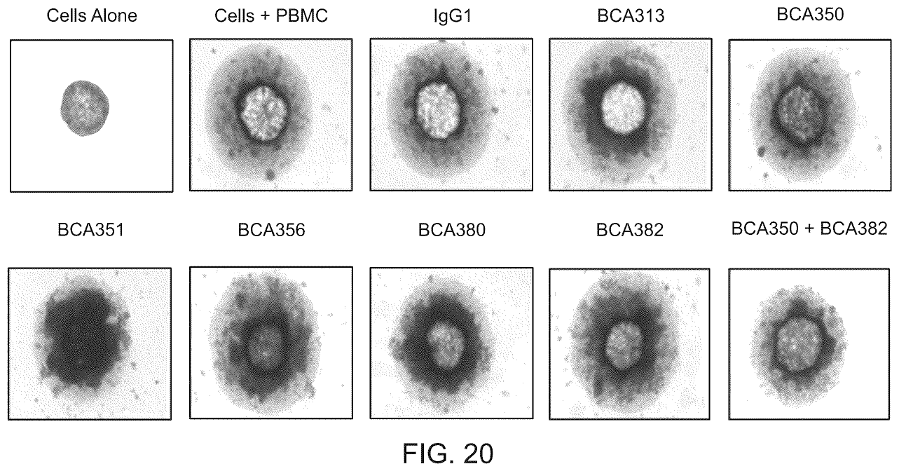

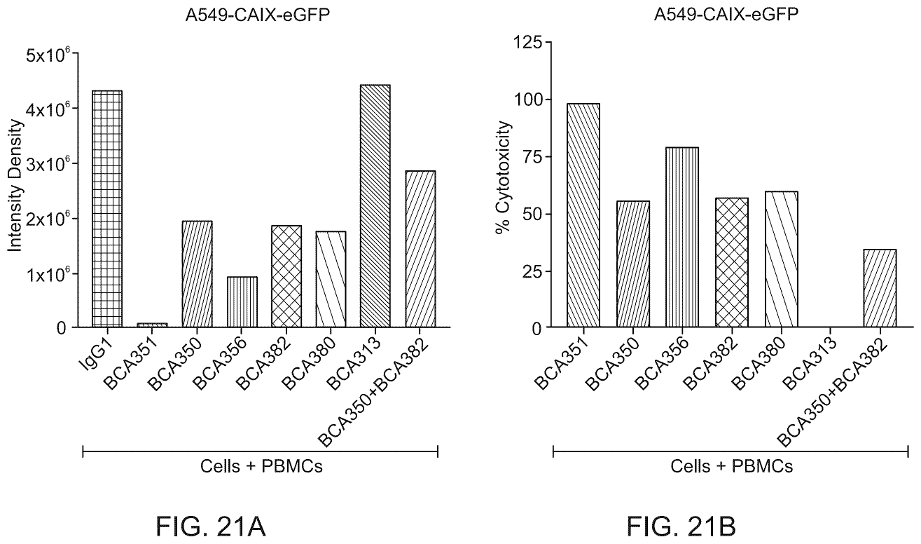

The A459 CAIX-eGFP expressing cells generated above were seeded in a U-bottom 96-well ULA plate and incubated four days to form spheroids. On Day 4, α-CD3 and α-CD28 antibody (1 μg/ml overnight) stimulated PBMCs (T: E at 1:10) and the indicated construct were added to the spheroid cultures and incubated for 72 hours. Subsequently bright field and florescence images were captured using Cytation 5 imager, representative images are presented in FIG. 20. A single eGFP spheroid from each group was quantified using Image J software and the data presented in FIG. 21A (A549 cell line eGFP Intensity Density) and FIG. 21B (Cytotoxicity—calculated considering IgG treated spheroid as 100% viability). As shown in FIGS. 21A-21B, the anti-CAIX hIL-12 fusion protein BCA356 (20 nM) has relatively higher cytotoxicity in the eGFP-CAIX expressing A549 spheroid model compared to BCA382 (a non-targeting IgG-attenuated IL-12 fusion protein) or BCA313 (a non-targeting Fc-attenuated IL-12 fusion protein).

### 6.14 Example 14. Anti-CAIX hIL-12 Variant Fusion Proteins Suppress Tumor Growth In Vivo with Limited Toxicity in Colon Cancer Model

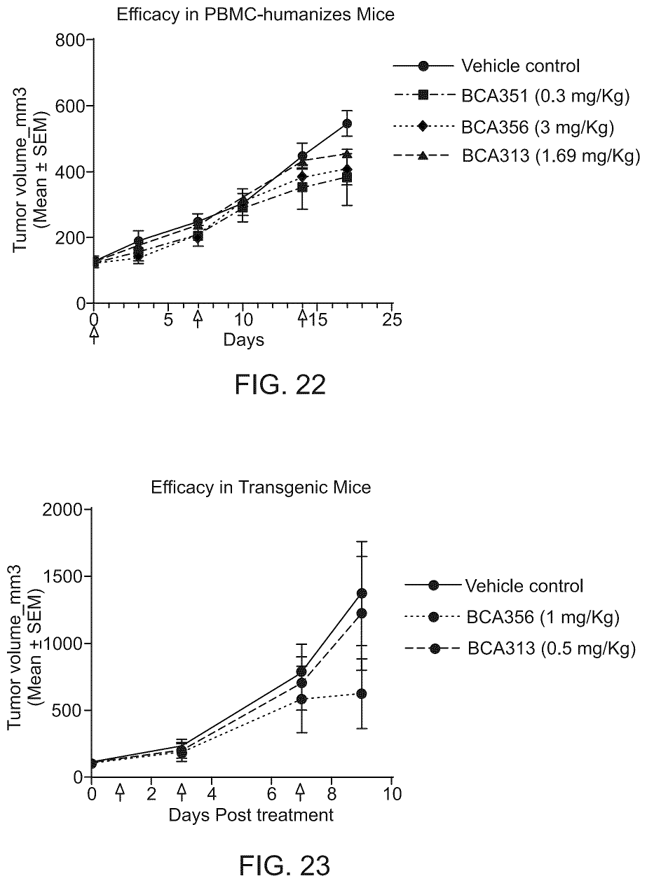

The efficacy of the anti-CAIX hIL-12 fusion protein BCA356 in the treatment of colon cancer was assessed using the HCT116-hCAIX tumor model. Briefly, approximately 5 million hCAIX transfected HCT116 cells were subcutaneously injected into NSG mice. When the tumors were palpable, about 10 million hPBMCs were injected intravenously into each mouse. When tumors were about 100 mm³, the mice were randomized into test and control groups and treated with one of the following constructs: vehicle control (phosphate buffered saline (PBS)), BCA351 (0.3 mg/kg), BCA356 (3 mg/kg) or BCA313 (1.69 mg/kg, equimolar dose) twice a week intraperitoneally. Tumor volumes, body weight and clinical signs were measured twice a week. Values were expressed as Mean±SEM of 6 mice per group (Table 29 and FIG. 22).

TABLE 29
Tumor Growth Inhibition and Death of Mice

| | % TGI | % Body weight | Deaths |
|---|---|---|---|
| Test Construct | (Mean + SD) | Change | Day17 |
| BCA351 | 30 ± 31 | −20 | 2/6 |
| BCA356 | 26 ± 21 | −14 | 0/6 |
| BCA313 | 16 ± 26 | −13 | 0/6 |

As show in Table 29 and FIG. 22, the anti-CAIX hIL-12 fusion protein BCA356 showed comparable tumor growth inhibition as the anti-CAIX hIL-12 fusion protein BCA351 (wild type hIL-12 fusion) (26% and 30% respectively), however, non-targeting Fc-IL-12 (BCA313) showed a reduced TGI (16%) compared to BCA351 or BCA356. Two mice were found dead in the BCA351 treated group on Day 17, whereas none of mice died in either of the BCA356 or BCA313 treated groups. Hence, the anti-CAIX hIL-12 fusion protein BCA356 is as efficacious as the anti-CAIX hIL-12 fusion protein BCA351 but with reduced toxicity. Study could not be extended beyond three doses due to onset of GvHD.

### 6.15 Example 15. Anti-CAIX hIL-12 Variant Fusion Proteins Suppress Tumor Growth In Vivo with Limited Toxicity in Melanoma Model

The efficacy of the anti-CAIX hIL-12 fusion protein BCA356 in the treatment of melanoma was assessed in the B16F10 expressing human CAIX allografts bearing hIL-12 and hIL-12 receptor knock-in transgenic mouse model. Briefly, about 0.1 million hCAIX expressing B16F10 cells were subcutaneously injected into transgenic mice subcutaneously. When the tumors were about 80 mm³, mice were randomized into test and control groups and treated with one of the following constructs: vehicle control (PBS), BCA356 (1 mg/kg) or BCA313 at equivalent dose (0.5 mg/kg), twice a week intraperitoneally. Tumor volumes, body weight and clinical signs were measured twice a week. Values are expressed as Mean±SEM of 6 mice per group (Table 30 and FIG. 23).

TABLE 30
Tumor Growth Inhibition and Death of Mice

| | % TGI | % Body | Deaths |
|---|---|---|---|
| Test Construct | (Mean + SD) | weight Change | Day9 |
| BCA356 | 55 ± 42 | 4 | 0/6 |
| BCA313 | 11 ± 69 | 9 | 1/6 |

As shown in Table 30 and FIG. 23, the anti-CAIX hIL-12 fusion protein BCA356 showed a trend of higher efficacy compared to BCA313 (55% and 11% respectively). One mouse found dead in BCA313 treated group on Day 9, whereas none of mice died in BCA356 treated groups. Hence, the anti-CAIX hIL-12 fusion protein BCA356 is efficacious and safe in the transgenic model.

### 6.16 Example 16. CAIX Expression in Normal and Cancer Tissues

The level of expression of human CAIX in human normal and cancer tissues was analyzed by immunohistochemistry. Briefly, CAIX protein expression in formalin fixed paraffin embedded (FFPE) four-micron tumor tissue sections on tumor microarrays (TMAs) having multiple tumor tissue types was evaluated by immunohistochemistry (IHC) using anti-CAIX antibody [Genetex (TH22)] on a Ventana Benchmark XT platform. The digital images captured from each TMA were analyzed using CellProfiler™ and the tumor areas in each core were annotated by a pathologist as regions of interest (ROI). The ROIs for each core were analyzed using a tuned membrane algorithm to get the intensity of CAIX staining for tumor cell membranes. Digital thresholds divided the pixel staining into 0+ (negative), 1+ (weak), 2+ (moderate) and 3+ (strong). The percentage of ROI staining in each category was used to calculate the final H-Score for each core using the following formula: H-Score=(1 X % 1+)+ (2 X % 2+)+ (3 X % 3+) (Maximum H-Score=300).

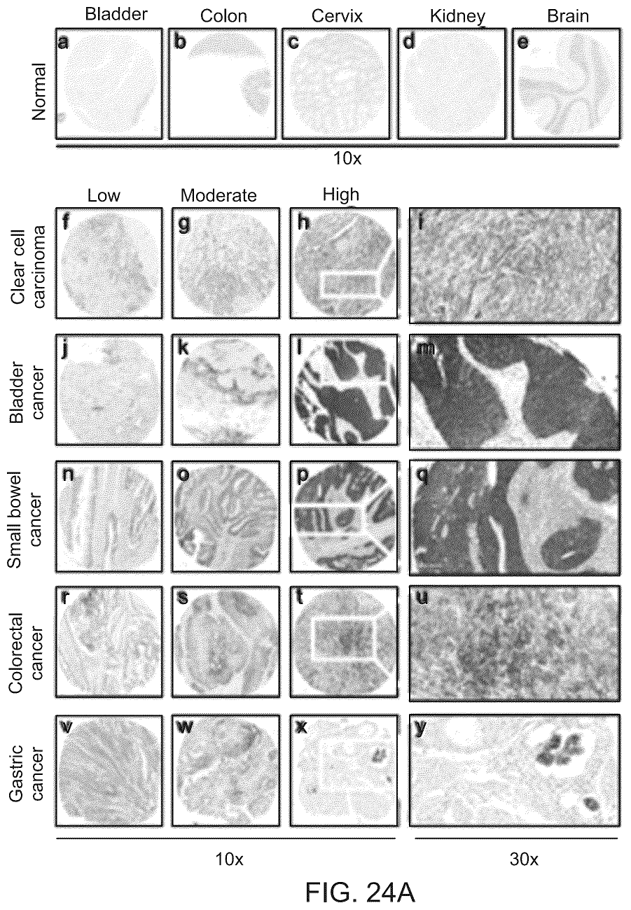

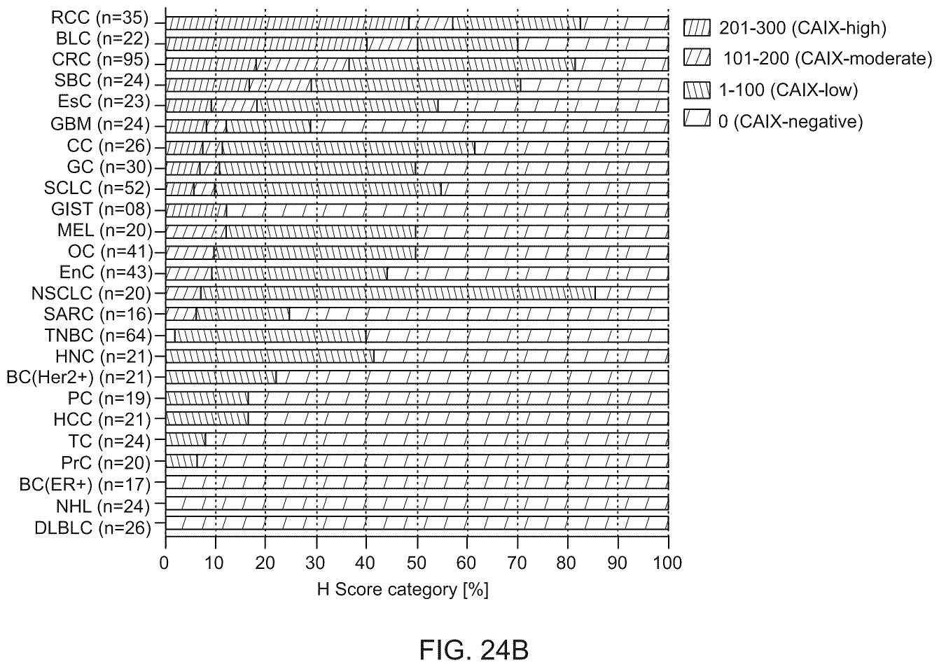

As shown in FIGS. 24A and 24B, the immunohistochemical analysis revealed that CAIX expression is absent in normal bladder, colon, cervix, kidney and brain tissues (FIG. 24A upper panel); and membranous CAIX expression is present in different solid tumor types (FIG. 24A lower panel, FIG. 24B). Evaluation of different patients of a cancer type reveal variable CAIX expression categorized as low (H score=1-100), moderate (101-200), and high (201-300) (FIG. 24B). High CAIX-expressing cases are observed in renal cell carcinoma, bladder cancer, colorectal cancer, small bowel cancer, esophageal/GEJ cancer, glioblastoma, cervical cancer, gastric cancer, non-small cell lung cancer, and gastrointestinal stromal tumor (FIG. 24A-24B). CAIX expression was shown to be absent in ER+ breast cancer, non-Hodgkin lymphoma, and Diffuse Large B-Cell Lymphoma (FIG. 24B).

### 6.17 Example 17. Lymphocyte Infiltration in CAIX-Expressing Tumors

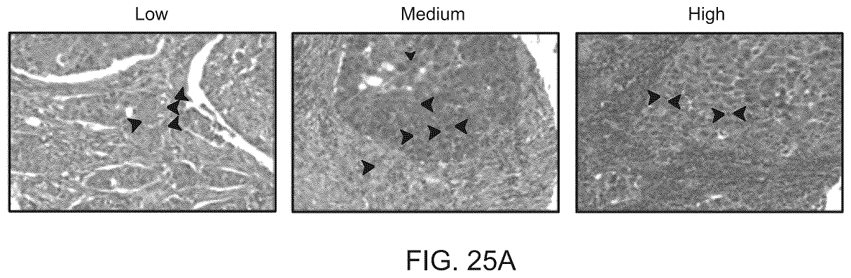

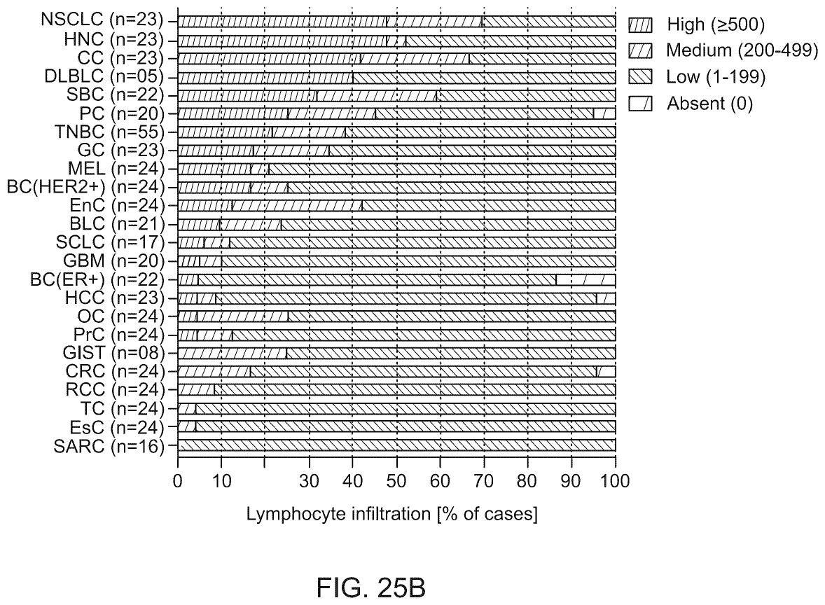

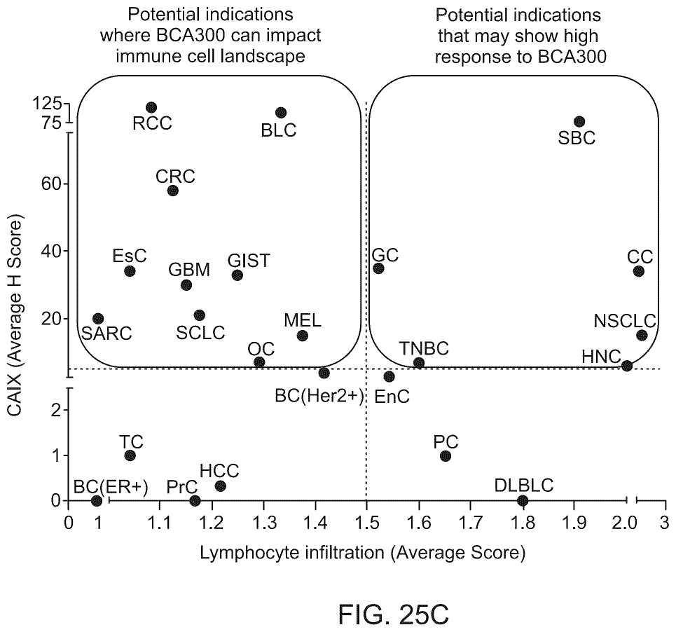

The level of lymphocyte infiltration in CAIX-expressing tumors was evaluated using hematoxylin & cosin (H&E) staining of tumor tissue cores. As shown in FIGS. 25A and 25B, in different H&E-stained tumor tissue cores, heterogeneous density of lymphocyte infiltration [absent (0), low (1-199), medium (200-499) and high (>500)] was observed (FIG. 25A-25B). Among CAIX-expressing tumors, the analysis revealed a low-density lymphocyte infiltration in renal cell carcinoma, bladder cancer, colorectal cancer, esophageal/GEJ cancer, gastrointestinal stromal tumor, glioblastoma, sarcoma, small cell lung cancer, melanoma, and ovarian cancer (FIG. 25C). Further, a medium density of lymphocyte infiltration was observed in small bowel cancer, gastric cancer, cervical cancer, non-small cell lung cancer, triple negative breast cancer, and head and neck cancer (FIG. 25C).

### 6.18 Example 18. Cell Specific Expression of CA9, IL12RB1, and IL12RB2 in Colorectal Adenocarcinoma

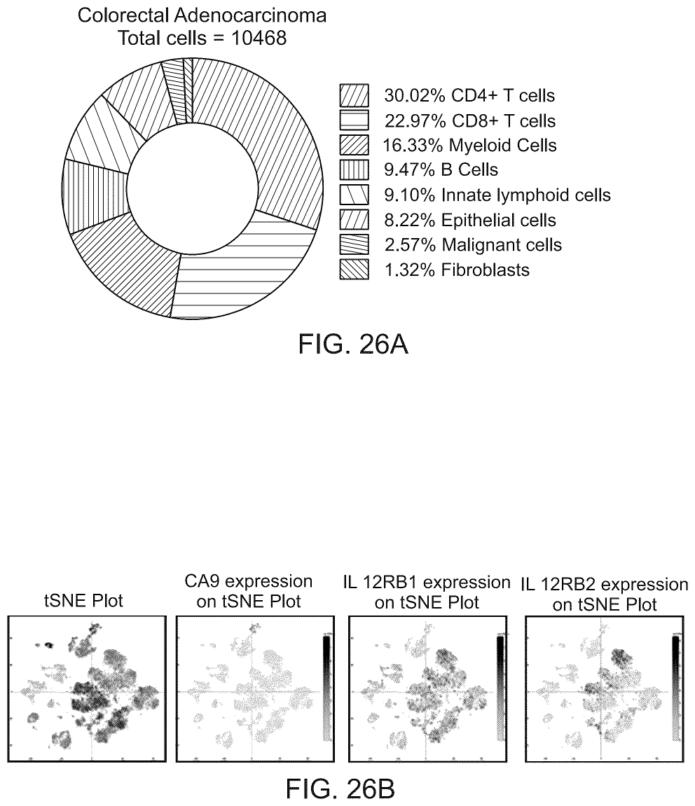

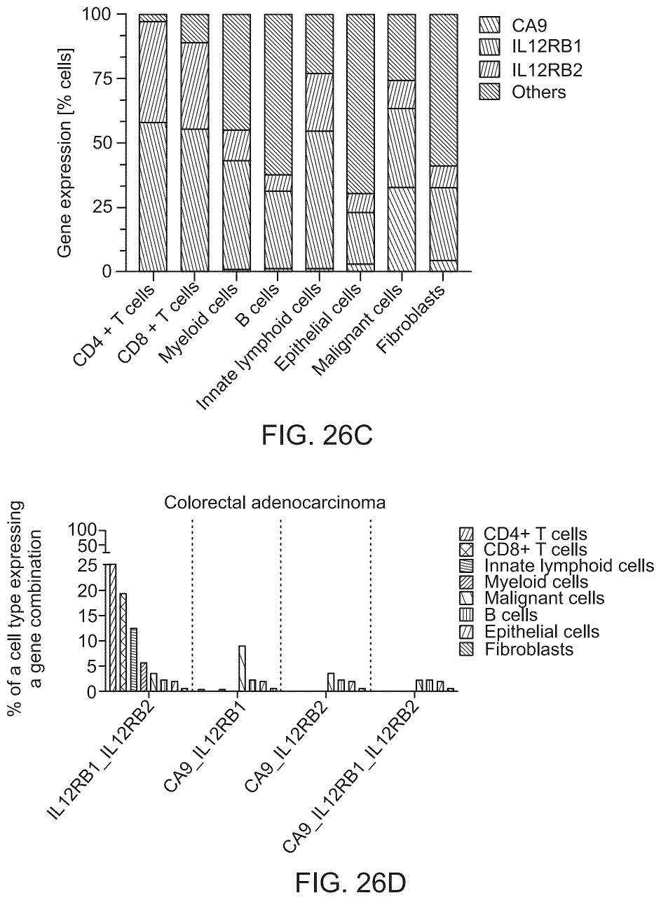

The types of cells expressing CA9, IL12RB1, and IL12RB2 in human cancer tissues was evaluated by analyzing the published single cell RNA-sequencing data of human colorectal adenocarcinoma tissues, see, Zhang, Lei et al. “Single-Cell Analyses Inform Mechanisms of Myeloid-Targeted Therapies in Colon Cancer.” Cell vol. 181, 2 (2020): 442-459.e29. doi:10.1016/j.cell.2020.03.048, the entire contents of which are incorporated herein by reference for all purposes. As shown in FIGS. 26A-26D, a representative example presented herein, single cell RNA-sequencing analysis of 10468 cells from treatment naïve colorectal adenocarcinoma patients (n=10) identified eight different cell clusters, including CD4+ T cells (30.02%), CD8+ T cells (22.97%), myeloid cells (16.33%), B cells (9.47%), innate lymphoid cells (9.10%), epithelial cells (8.22%), malignant cells (2.57%), and fibroblasts (1.32%) (FIGS. 26A and 26B). Among different cell types, CA9 expression was prominently observed in malignant cells (32.7%), whereas its expression was absent in all immune cell types (FIGS. 26B and 26C). Extensive expression of IL12RB1 was observed in CD4+ (57.4%), CD8+ (55%) T cells, and innate lymphoid cells (53.3%) (FIGS. 26B and 26C). IL12RB2 expression was observed in CD4+ (39%) and CD8+ (33.5%) T cells (FIGS. 26B and 26C). Further analysis revealed that co-expression of IL12RB1 and IL12RB2 was present in 25.23% of CD4+ and 19.37% of CD8+ T cells (FIG. 26D).

Furthermore, analysis of data from single cell RNA-sequencing from a public database on gastric adenocarcinoma, glioblastoma, head and neck cancer, lung cancer, melanoma, nasopharyngeal carcinoma, and ovarian cancer patients revealed CA9 expression prominently in malignant epithelial cells. The IL12RB1 was determined to be constitutively expressed in multiple cell types with prominent expression T cells, NK cells, innate lymphoid cells, and myeloid cells. Compared to the IL12RB1 expression, the IL12RB2 expression was observed in lower percentage of T cells and NK cells. Across the tumor types, very low percentage of tumor cells co-express CAIX, IL12RB1 and IL12RB2 transcripts.

### 6.19 Example 19. Summary-Attenuation of hIL-12 Proteins Comprising Variant hIL-12p40 Polypeptides

Table 31 provides a summary of the attenuation of hIL-12 proteins comprising different variant hIL-12p40 polypeptides described herein, as assessed by one or more of the assays described in Examples 4-18.

TABLE 31
Summary of Attenuation of hIL-12 Proteins Comprising
hIL-12p40 Variants

| | | | Enriched | PSTAT | |
|---|---|---|---|---|---|
| | | PBMC | NK | (CD8) | |
| | HEK | Assay | cell | flow | |
| | IL-12 | (PHA- | assay- | cytometry- | |
| | Reporter | stimulated) | percent | percent | |
| | Assay | Percent | reduction | reduction | |
| | (Ratio | Reduction | at | at | Modi- |
| Description | of EC50) | at 16.9 nM | 16.9 nM | 16.9 nM | fications |
| BCA331.16 | 7300.00 | 70.16 | 86.95 | 91.07 | W37A, |
| (BCA356) | (+++) | (++++) | (++++) | (++++) | F82A, |
| | | | | | K219A |
| BCA327.16 | 9566.00 | 69.34 | 82.88 | 88.19 | E81A, |
| | (+++) | (++++) | (++++) | (+++) | F82A, |
| | | | | | K106A |
| BCA310.16 | 22740.00 | 77.04 | 97.73 | 98.81 | D1 |
| | (+++++) | (+++++) | (+++++) | (+++++) | Domain |
| | | | | | Deleted |
| BCA313 | 4996.00 | 68.66 | 70.16 | 82.50 | E81K, |
| (Positive | (+++) | (++++) | (+++) | (+++) | K121E, |
| Control) | | | | | K286E |
| BCA330.16 | 107.00 | 61.98 | 40.98 | NT | W37A, |
| | (+) | (+++) | (++) | | F82A, |
| | | | | | K217A |
| BCA329.16 | 4686.00 | 54.17 | 73.31 | 72.86 | P39A, |
| | (+++) | (++) | (+++) | (++) | D40A, |
| | | | | | E81A, |
| | | | | | F82A |
| BCA321.16 | 6594.00 | 74.35 | 93.45 | 93.87 | K217- |
| | (+++) | (+++++) | (+++++) | (++++) | L218 |
| | | | | | to |
| | | | | | 217I |
| BCA332.16 | 141.00 | 45.85 | NT | NT | K106A, |
| | (+) | (+++++) | | | K217A, |
| | | | | | K219A |
| BCA326.16 | 374.00 | 34.31 | −13.81 | 50.24 | E81A, |
| | (+) | (+) | (no | (+) | F82A |
| | | | atten- | | |
| | | | uation) | | |

The least attenuated are indicated with a “+” while the maximum attenuation is indicated with “+++++”.
NT indicates not tested.

hIL-12 variants comprising F82A or/and E81A amino acid substitutions in the hIL-12p40 subunit do not suggest as being the driver mutations for hIL-12 attenuation. For example, E81A/F82A (BCA326.16) is the least attenuated while BCA329.16 is moderately attenuated. hIL-12 proteins comprising a hIL-12p40 subunit comprising W37A, F82A, K219A amino acid substitutions (BCA331.16 (also referred to herein as BCA356)) is more attenuated than those containing W37A, F82A, K217A amino acid substitutions (BCA330.16), unexpectedly suggesting a critical role for amino acid position K219 while a different cluster of E81A, F82A, K106A (BCA327.16) is similar to (BCA331.16 (also referred to herein as BCA356)).

### 6.20 Example 20. Generation of Anti-CAIX Fusion Proteins Comprising Variant hIL-12p35 Proteins

The following anti-hCAIX hIL-12 fusion proteins were generated according to standard methods. Briefly, the different polypeptide sequence of each hIL-12p35 variant was codon optimized to obtain a DNA polynucleotide counterpart that was cloned into a suitable mammalian expression plasmid. The respective plasmids were transiently co-transfected into cell line. The transfected cells were grown for serval days with appropriate feeding as per the manufacturer's protocol. The supernatants of the cell cultures were harvested and the hIL-12p35 proteins purified.

Table 32 provides a description of the amino acid modifications contained in the hIL-12 polypeptide of the generated fusion proteins.

TABLE 32
The Amino Acid Sequence of hIL-12p35 Variants

| | hIL-12p35 Amino Acid Substitution | |
|---|---|---|
| | (numbering relative to the amino | SEQ ID |
| Description | acid sequence of SEQ ID NO: 30) | NOS |
| BCA325.16 | Y189A | 304, 311, 245 |
| BCA324.16 | F188A | 304, 310, 245 |
| BCA323.16 | F188A/Y189A | 304, 309, 245 |
| BCA307.16 | 455-92 | 304, 307, 245 |
| BCA308.16 | E60K/F61H/P63S/K150H/F188P | 304, 308, 245 |

Table 33 provides a description and the amino acid sequence of each hIL-12p35 variant generated. The signal sequence of each polypeptide chain is underlined.

TABLE 33
The Amino Acid Sequence of Anti-hCAIX hIL-12 Fusion Proteins

| | Description | SEQ ID NO |
|---|---|---|
| BCA325.16 | Heavy Chain (Knob) | 304 |
| | IL-12p40 | |
| | Heavy Chain (Hole) | 311 |
| | IL-12p35 (variant E) | |
| | Light Chain | 245 |
| BCA324.16 | Heavy Chain (Knob) | 304 |
| | IL-12p40 | |
| | Heavy Chain (Hole) | 310 |
| | IL-12p35 (variant D) | |
| | Light Chain | 245 |
| BCA323.16 | Heavy Chain (Knob) | 304 |
| | IL-12p40 | |
| | Heavy Chain (Hole) | 309 |
| | IL-12p35 (variant C) | |
| | Light Chain | 245 |
| BCA307.16 | Heavy Chain (Knob) | 304 |
| | IL-12p40 | |
| | Heavy Chain (Hole) | 307 |
| | IL-12p35 (variant A) | |
| | Light Chain | 245 |
| BCA308.16 | Heavy Chain (Knob) | 304 |
| | IL-12p40 | |
| | Heavy Chain (Hole) | 308 |
| | IL-12p35 (variant B) | |
| | Light Chain | 245 |

### 6.21 Example 21. Evaluation of hIL-12p35 Variant Activity-pSTAT4 Signaling

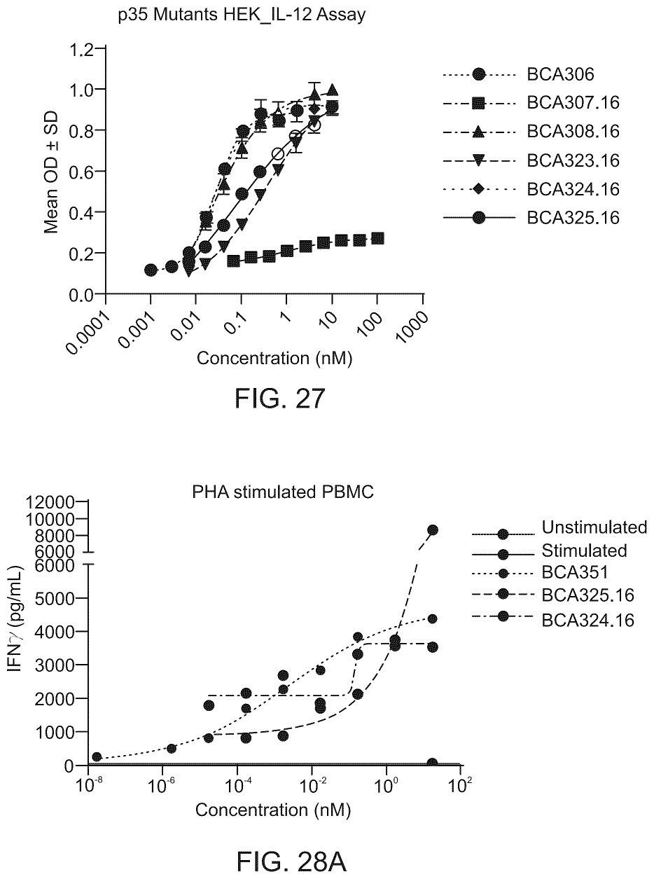

Signaling through the hIL-12 receptor of each of the hIL-12p35 variants set forth in Table 32 and 33 was evaluated using a HEK-Blue™ Reporter assay (InvivoGen (#hkb-IL12)). Briefly, HEK-Blue™ IL-12 cells utilized in the assay express a STAT4-inducible SEAP reporter gene. The binding of IL-12 to the IL-12R on the surface of HEK-Blue™ IL-12 cells triggers a signalling cascade leading to the activation (and phosphorylation) of STAT-4 with the subsequent production of the SEAP reporter protein. Detection of SEAP in the supernatant of HEK-Blue™ IL-12 cells can be readily assessed using QUANTI-Blue™ Solution. Here, HEK-Blue cells were incubated with various concentrations of each of the indicated hIL-12p35 variants for 18-24 hours in cell culture plates. The plates were read after addition of Quanti Blue solution. The IL-12 activity of hIL-12p35 variants is presented in FIG. 27 and the fold attenuation of IL-12 activity over wild type Fc-IL-12 is presented in Table 34.

TABLE 34
Fold Attenuation of hIL-12p35 Variants over Fc-IL-12 Control

| | *Fold Attenuation |
|---|---|
| Description | (Over Fc-IL-12 Control) |
| BCA307.16 | >10,000* |
| BCA308.16 | 1.4 |
| BCA323.16 | 15.4 |
| BCA324.16 | 1.0 |
| BCA325.16 | 4.5 |

Fold attenuation of IL-12 activity for BCA307.16 could not be determined as tested concentration did not achieve sufficient IL-12 activity in HEK assay.

As shown in FIG. 27 and Table 34, each of the hIL-12p35 variants showed a hIL-12 activity attenuation ranging from one-fold to 15-fold, except BCA307.16 which showed more than 10,000-fold attenuation.

### 6.22 Example 22. Evaluation of hIL-12p35 Variant Activity-IFN-γ Release by PHA Stimulated PBMCs

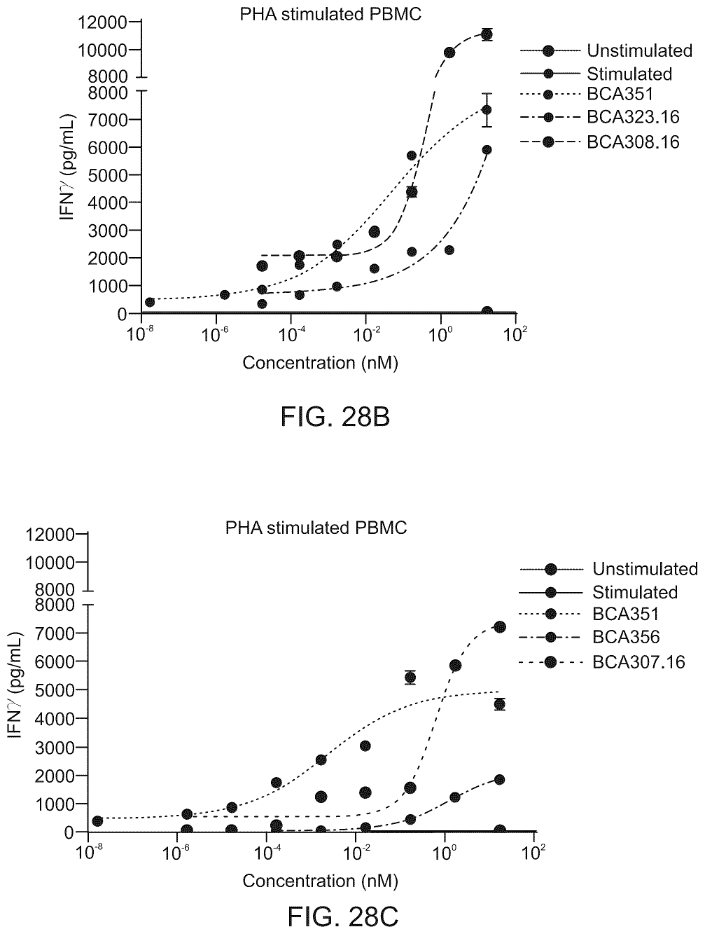

The level of IFN-γ released by T-cells stimulated in the presence of each of the hIL-12p35 variants was evaluated. Briefly, hPBMCs were seeded at 0.1 million cells/well in a 96 well plate and stimulated overnight with PHA (1 μg/ml) in a volume of 100 μl. The following day, PHA was added again at the same concentration along with titrating concentration of the indicated hIL-12p40 variant. The cells were subsequently incubated at 37° C. in a 5% CO₂ incubator for ˜48 hours. IFN-γ was measured in the culture supernatant by a standard IFN-γ ELISA (FIGS. 28A-28C). As shown in FIG. 28A-28C, BCA325.16, BCA323.16 and BCA307.16 exhibited attenuated IFN-γ release at 0.169 nM (not at higher concentrations). BCA324.16, BCA308.16 do not show attenuated IFN-γ release. BCA356, used as a positive control, showed the maximum attenuation in IFN-γ release.

### 6.23 Example 23. Evaluation of hIL-12p35 Variant Activity-IFN-γ Release by hIL-2 Primed Human NK Cells

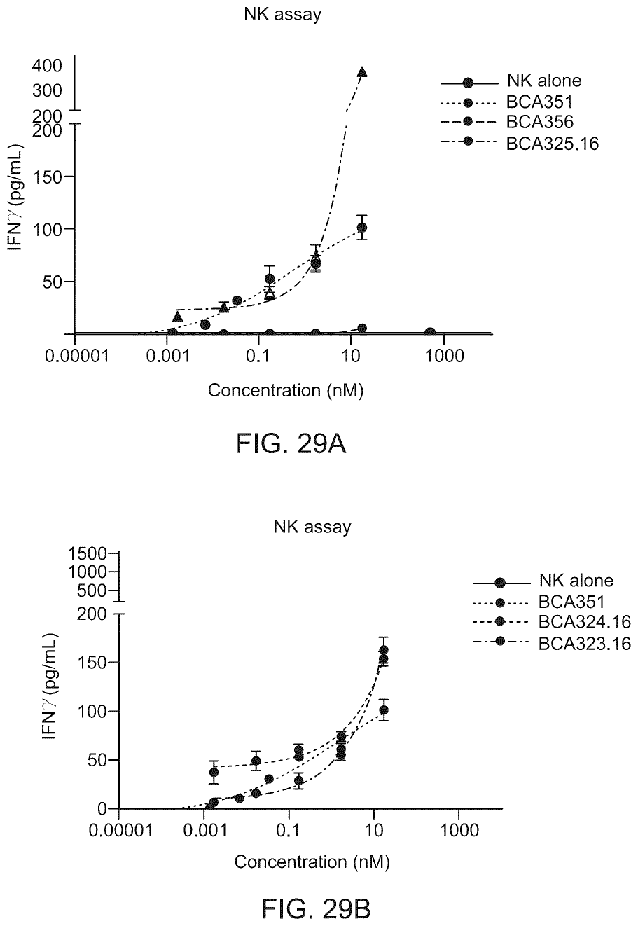

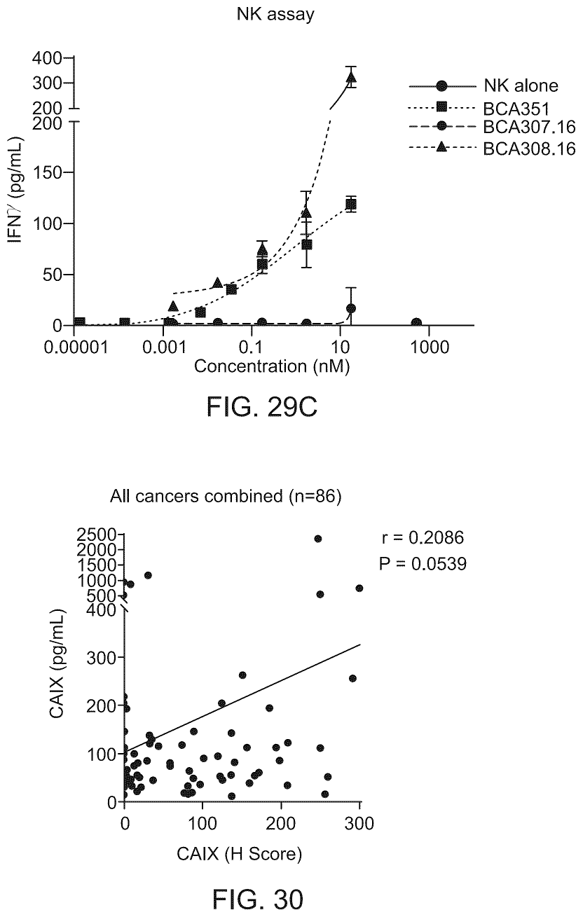

IL-12 stimulates NK cells to secrete IFN-γ and augments their proliferation and cytolytic activity (see, e.g., Glassman, Caleb R et al. “Structural basis for IL-12 and IL-23 receptor sharing reveals a gateway for shaping actions on T versus NK cells.” Cell vol. 184, 4 (2021): 983-999.e24. doi: 10.1016/j.cell.2021.01.018). To evaluate the level of IFN-γ released from NK cells, hPBMCs were thawed in RPMI 1640 containing 10% HI-FBS and 100 IU/ml hIL-2. After overnight incubation the cells were harvested, and the NK cells were enriched by negative selection. NK cells were seeded as 50,000 cells/well along with titrating concentration of hIL-12 and incubated at 37° C. in a 5% CO₂ for ˜48 hours. IFN-γ was measured in the culture supernatant by IFN-γ a standard IFN-γ ELISA (FIGS. 29A-29C). As shown in FIGS. 29A-29C, BCA307.16 showed attenuation comparable to BCA356 in IFN-γ release at 16.95 nM; BCA325.16, BCA323.16, BCA324.16 and BCA308.16 do not show attenuation in IFN-γ release.

### 6.24 Example 24. Summary hIL-12p35 Variant Activity

The data presented in Examples 19-22 and summarized in Table 35 shows that hIL-12 in BCA307 is >10000 fold attenuated compared to wild type hIL-12 in HEK-IL-12 reporter assay; and. BCA307 is highly attenuated in NK cell-based assays when compared to other hIL-12p35 variants.

TABLE 35
Summary of Functional Analysis of hIL-12p35 Variants

| | | | | IFNγ release |
|---|---|---|---|---|
| | | | IFNγ | by PHA |
| | | HEK-IL12 | release by | stimulated |
| | | reporter | NK cells | PBMC |
| | Amino Acid | assay | *Level of attenuation based | |
| Description | Modifications | Attenuation | on shifts in curves | |
| BCA307.16 | 455-92 | +++ | +++ | + |
| BCA308.16 | E60K/ | Not | not attenuated | not attenuated |
| | F61H/P63S/ | attenuated | | |
| | K150H/F188P | | | |
| BCA323.16 | F188A/Y189A | + | not attenuated | + |
| BCA324.16 | F188A | not attenuated | not attenuated | not attenuated |
| BCA325.16 | Y189A | not attenuated | not attenuated | + |

*Qualitative ranking done based on shifts in curves where +, ++, +++ represents low, mid and high attenuation compared to IL-12 WT.

### 6.25 Example 25. Soluble CAIX as a Surrogate Biomarker

To determine utility of the soluble CAIX level as a surrogate biomarker of expression of CAIX in a tumor, cellular CAIX expression in tumor tissues and soluble CAIX levels in tumor-matched plasma were determined in samples from gastric, kidney, colorectal, ovarian, non-small cell lung, and endometrial cancer cases by immunohistochemistry (IHC) and enzyme linked immuno-sorbent assay (ELISA), respectively. As shown in FIG. 30, in a Pearson's correlation coefficient analysis of all cancers combined together, a positive correlation (r=0.2086, P=0.0539) was observed between cellular and soluble CAIX.

The invention is not to be limited in scope by the specific embodiments described herein. Indeed, various modifications of the invention in addition to those described will become apparent to those skilled in the art from the foregoing description and accompanying figures. Such modifications are intended to fall within the scope of the appended claims.

All references (e.g., publications or patents or patent applications) cited herein are incorporated herein by reference in their entireties and for all purposes to the same extent as if each individual reference (e.g., publication or patent or patent application) was specifically and individually indicated to be incorporated by reference in its entirety for all purposes.

Other embodiments are within the following claims.

1. A human interleukin 12 p40 (hIL-12p40) polypeptide comprising an amino acid sequence (a) at least 85%, 86%, 87%, 88%, 89%, 90%, 91%, 92%, 93%, 94%, 95%, 96%, 97%, 98%, or 99% identical to the amino acid sequence of SEQ ID NO: 33; and (b) comprising or consisting of an amino acid substitution at each of amino acid positions (i) W37, F82, and K219; (ii) W37, F82, and K217; (iii) K106, K217, and K219; (iv) W37 and F82; (v) W37 and K217; (vi) W37 and K219; (vii) W37 and K106; (viii) F82 and K106; (xiv) F82 and K217; (xv) F82 and K219; (xvi) K217 and K219; (xvii) K106 and K217; or (xviii) K106 and K219, amino acid numbering relative to the amino acid sequence of SEQ ID NO: 32.

2-16. (canceled)

17. A human interleukin 12 p35 (hIL-12p35) polypeptide comprising an amino acid sequence (a) at least 85%, 86%, 87%, 88%, 89%, 90%, 91%, 92%, 93%, 94%, 95%, 96%, 97%, 98%, or 99% identical to the amino acid sequence of SEQ ID NO: 31; and (b) comprises or consists of an amino acid modification at one or more of the following amino acid positions E60, F61, P63, K150, F188, Y189A, amino acid numbering relative to the amino acid sequence of SEQ ID NO: 30.

18-21. (canceled)

22. A hIL-12p35 polypeptide, wherein the amino acid sequence of the hIL-12p35 polypeptide comprises or consists of a deletion of amino acids A55-K92, N50-K92, M51-K92, L52-K92, Q53-K92, K54-K92, N50-N93, M51-N93, L52-N93, Q53-N93, K54-N93, N50-E94, M51-E94, L52-E94, Q53-E94, K54-E94, N50-S95, M51-S95, L52-S95, Q53-S95, K54-S95, N50-C96, M51-C96, L52-C96, Q53-C96, K54-C96, N50-L97, M51-L97, L52-L97, Q53-L97, K54-L97, N50-P87, M51-P87, L52-P87, Q53-P87, K54-P87, N50-L88, M51-L88, L52-L88, Q53-L88, K54-L88, N50-E89, M51-E89, L52-E89, Q53-E89, K54-E89, N50-L90, M51-L90, L52-L90, Q53-L90, K54-L90, N50-T91, M51-T91, L52-T91, Q53-T91, K54-T91, R56-K92, Q57-K92, T58-K92, L59-K92, E60-K92 A55-N93, R56-N93, Q57-N93, T58-N93, L59-N93, E60-N93, A55-E94, R56-E94, Q57-E94, T58-E94, L59-E94, E60-E94, A55-S95, R56-S95, Q57-S95, T58-S95, L59-S95, E60-S95, A55-C96, R56-C96, Q57-C96, T58-C96, L59-C96, E60-C96, A55-L97, R56-L97, Q57-L97, T58-L97, L59-L97, E60-L97, A55-P87, R56-P87, Q57-P87, T58-P87, L59-P87, E60-P87, A55-L88, R56-L88, Q57-L88, T58-L88, L59-L88, E60-L88, A55-E89, R56-E89, Q57-E89, T58-E89, L59-E89, E60-E89, A55-L90, R56-L90, Q57-L90, T58-L90, L59-L90, E60-L90, A55-T91, R56-T91, Q57-T91, T58-T91, L59-T91, or E60-T91 (amino acid numbering relative to the amino acid sequence of SEQ ID NO: 30), and other than the deletion of amino acids A55-K92, N50-K92, M51-K92, L52-K92, Q53-K92, K54-K92, N50-N93, M51-N93, L52-N93, Q53-N93, K54-N93, N50-E94, M51-E94, L52-E94, Q53-E94, K54-E94, N50-S95, M51-S95, L52-S95, Q53-S95, K54-S95, N50-C96, M51-C96, L52-C96, Q53-C96, K54-C96, N50-L97, M51-L97, L52-L97, Q53-L97, K54-L97, N50-P87, M51-P87, L52-P87, Q53-P87, K54-P87, N50-L88, M51-L88, L52-L88, Q53-L88, K54-L88, N50-E89, M51-E89, L52-E89, Q53-E89, K54-E89, N50-L90, M51-L90, L52-L90, Q53-L90, K54-L90, N50-T91, M51-T91, L52-T91, Q53-T91, K54-T91, R56-K92, Q57-K92, T58-K92, L59-K92, E60-K92 A55-N93, R56-N93, Q57-N93, T58-N93, L59-N93, E60-N93, A55-E94, R56-E94, Q57-E94, T58-E94, L59-E94, E60-E94, A55-S95, R56-S95, Q57-S95, T58-S95, L59-S95, E60-S95, A55-C96, R56-C96, Q57-C96, T58-C96, L59-C96, E60-C96, A55-L97, R56-L97, Q57-L97, T58-L97, L59-L97, E60-L97, A55-P87, R56-P87, Q57-P87, T58-P87, L59-P87, E60-P87, A55-L88, R56-L88, Q57-L88, T58-L88, L59-L88, E60-L88, A55-E89, R56-E89, Q57-E89, T58-E89, L59-E89, E60-E89, A55-L90, R56-L90, Q57-L90, T58-L90, L59-L90, E60-L90, A55-T91, R56-T91, Q57-T91, T58-T91, L59-T91, or E60-T91 the amino acid sequence of the polypeptide is at least 85%, 86%, 87%, 88%, 89%, 90%, 91%, 92%, 93%, 94%, 95%, 96%, 97%, 98%, or 99% identical to the amino acid sequence of SEQ ID NO: 31.

23.-24. (canceled)

25. A single chain hIL-12 (schIL-12) polypeptide comprising the hIL-12p40 polypeptide of claim 1 operably connected to a hIL-12p35 polypeptide.

26.-28. (canceled)

29. A schIL-12 polypeptide comprising the hIL-12p35 polypeptide of claim 17 operably connected to a hIL-12p40 polypeptide.

30.-35. (canceled)

36. A fusion protein comprising
a. the hIL-12p40 polypeptide of claim 1,
b. a hIL-12p35 polypeptide; and
c. a heterologous moiety.

37. A fusion protein comprising
a. a hIL-12p40 polypeptide;
b. the hIL-12p35 polypeptide of claim 17; and
c. a heterologous moiety.

38.-117. (canceled)

118. A fusion protein comprising
a. a full-length antibody that specifically binds a hTAA comprising:
i. a first light chain comprising from N- to C-terminus a light chain variable region (VL) region and a light chain constant region (CL) region;
ii. a first heavy chain comprising from N- to C-terminus a heavy chain variable region (VH) region, a CH1 region, a hinge region, a CH2 region, and a CH3 region;
iii. a second heavy chain comprising from N- to C-terminus a VH region, a CH1 region, a hinge region, a CH2 region, and a CH3 region;
iv. a second light chain comprising from N- to C-terminus a VL region and a VH region;
wherein the first light chain and the first heavy chain associate to form a first antigen binding domain;
wherein the second light chain and the second heavy chain associate to form a second antigen binding domain; and
wherein the first heavy chain and the second heavy chain associate to form a dimer;
b. the hIL-12p40 polypeptide of claim 1,
c. a hIL-12p35 polypeptide;
wherein the CH3 region of the first heavy chain of the full-length antibody comprises one or more amino acid modification relative to the amino acid sequence of a reference CH3 region that does not contain the one or more amino acid modification;
wherein the CH3 region of the second heavy chain of the of the full-length antibody comprises one or more amino acid modification relative to the amino acid sequence of a reference CH3 region that does not contain the one or more amino acid modification;
wherein the one or more amino acid modification in the CH3 region of the first heavy chain of the full-length antibody is different from the one or more amino acid modification in the CH3 region of the second heavy chain of the full-length antibody;
wherein the one or more amino acid modification in the CH3 region of the first heavy chain of the full-length antibody and the one or more amino acid modification in the CH3 region of the second heavy chain of the full-length antibody promote heterodimerization of the first and second heavy chain of the full-length antibody;
wherein the N-terminus of the hIL-12p40 polypeptide is operably connected to the C-terminus of the CH3 region of the first heavy chain via a first peptide linker; and
wherein the N-terminus of the hIL-12p35 polypeptide is operably connected to the C-terminus of the CH3 region of the second heavy chain via a second peptide linker.

119.-130. (canceled)

131. A fusion protein comprising:
a. a first polypeptide comprising a first light chain comprising from N- to C-terminus a VL region and a CL region;
b. a second polypeptide comprising from N- to C-terminus: (i) a first heavy chain comprising from N- to C-terminus a VH region, a CH1 region, a hinge region, a CH2 region, and a CH3 region; (ii) a first peptide linker, and (iii) the hIL-12p40 polypeptide of claim 1;
c. third polypeptide comprising from N- to C-terminus: (i) a second heavy chain comprising from N- to C-terminus a VH region, a CH1 region, a hinge region, a CH2 region, and a CH3 region, (ii) a second peptide linker; and (iii) a hIL-12p35 polypeptide; and
d. a fourth polypeptide comprising a second light chain comprising from N- to C-terminus a VL region and a CL region;
wherein the VL of the first light chain and the VH of the first heavy chain associate to form a first antigen binding domain that specifically binds a first hTAA;
wherein the CH3 region of the first heavy chain comprises one or more amino acid modification relative to the amino acid sequence of a reference CH3 region that does not contain the one or more amino acid modification;
wherein the CH3 region of the second heavy chain comprises one or more amino acid modification relative to the amino acid sequence of a reference CH3 region that does not contain the one or more amino acid modification;
wherein the one or more amino acid modification in the CH3 region of the first heavy chain of the full-length antibody is different from the one or more amino acid modification in the CH3 region of the second heavy chain of the full-length antibody;
wherein the one or more amino acid modification in the CH3 region of the first heavy chain of the full-length antibody and the one or more amino acid modification in the CH3 region of the second heavy chain of the full-length antibody promote heterodimerization of the first and second heavy chain of the full-length antibody.

132.-143. (canceled)

144. An antibody (or antigen binding domain thereof) that specifically binds hCAIX and comprises a VH and VL, wherein the amino acid sequence of the VH is at least 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 3-9; and the amino acid sequence of the VL is at least 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 10-17.

145. (canceled)

146. A polynucleotide encoding the hIL-12p40 polypeptide of claim 1.

147.-148. (canceled)

149. An expression vector comprising the polynucleotide of claim 146.

150. (canceled)

151. A host cell comprising the hIL-12p40 polypeptide of claim 1.

152. A carrier comprising the hIL-12p40 polypeptide of claim 1.

153. (canceled)

154. A pharmaceutical composition comprising the hIL-12p40 polypeptide of claim 1, and a pharmaceutically acceptable excipient.

155. A kit comprising the hIL-12p40 polypeptide of claim 1.

156. A method of making the hIL-12p40 polypeptide of claim 1, the method comprising:
a. introducing into a population of in vitro or ex vivo cells a polynucleotide encoding the hIL-12p40 polypeptide,
b. culturing the population of cells under conditions sufficient for the population of cells to express the multispecific protein; and
c. optionally isolating and/or purifying the hIL-12p40 polypeptide.

157. A method of delivering a polypeptide to a subject, the method comprising administering the hIL-12p40 polypeptide of claim 1 to the subject, in an amount and for a time sufficient to deliver the hIL-12p40 polypeptide to the subject.

158. A method of stimulating T-cell or NK cell effector function in a subject, the method comprising administering the hIL-12p40 polypeptide of claim 1 to the subject, in an amount and for a time sufficient to stimulate T-cell or NK cell effector function in the subject.

159. A method of preventing or treating a cancer in a subject, the method comprising administering the hIL-12p40 polypeptide of claim 1 to the subject in need thereof, in an amount and for a time sufficient to prevent or treat the cancer in the subject.

160.-162. (canceled)

163. A method of determining the expression of CAIX in cells of a cancer in a subject, the method comprising:
a. obtaining the sample from a subject, wherein the sample does not contain cancer cells (or does not contain a substantial number of cancer cells), and
b. determining the presence or absence of soluble CAIX (or a fragment or variant thereof) in the sample.

164. A method of diagnosing a subject with a cancer comprising cancer cells expressing CAIX, the method comprising:
a. obtaining the sample from a subject, wherein the sample does not contain cancer cells (or does not contain a substantial number of cancer cells),
b. determining the presence or absence of soluble CAIX (or a fragment or variant thereof) in the sample; and
c. diagnosing the subject as having a cancer comprising cancer cells expressing CAIX, if soluble CAIX is determined to be present in the sample.

165. A method of treating a cancer in a subject, the method comprising:
a. receiving test results that determined the presence of soluble CAIX in a sample from a subject, wherein the sample does not contain cancer cells (or does not contain a substantial number of cancer cells);
b. diagnosing the subject as having a cancer comprising cancer cells expressing CAIX; and
c. administering the hIL-12p40 polypeptide of claim 1 to the subject in need thereof, in an amount and for a time sufficient to treat the cancer in the subject.

166.-169. (canceled)

170. A fusion protein comprising
a. a hIL-12p40 polypeptide;
b. the hIL-12p35 polypeptide of claim 22; and
c. a heterologous moiety.
